SEQUENCE LISTING (61 sequences)

SEQ ID NO: 1 - DNA, 20 bp, synthetic construct
  source 1..20: mol_type = other DNA
  modified_base 10: mod_base = OTHER; note = 2'-O-(2-oxo-2-(ethyloxy)ethyl)uridine-3'-phosphate
  misc_feature 10..20: note = RNA
  misc_feature 1..9: note = DNA
tttttttttttttttttttt

SEQ ID NO: 2 - DNA, 20 bp, synthetic construct
  source 1..20: mol_type = other DNA
  modified_base 10: mod_base = OTHER; note = 2'-O-(2-oxo-2-(ethyloxy)ethyl)cytidine-3'-phosphate
  misc_feature 10..20: note = RNA
  misc_feature 1..9: note = DNA
tttttttttctttttttttt

SEQ ID NO: 3 - DNA, 20 bp, synthetic construct
  source 1..20: mol_type = other DNA
  modified_base 10: mod_base = OTHER; note = 2'-O-(2-oxo-2-(ethyloxy)ethyl)adenosine-3'-phosphat e
  misc_feature 10..20: note = RNA
  misc_feature 1..9: note = DNA
tttttttttatttttttttt

SEQ ID NO: 4 - DNA, 20 bp, synthetic construct
  source 1..20: mol_type = other DNA
  modified_base 10: mod_base = OTHER; note = 2'-O-(2-oxo-2-(N-hexadec-1-ylamino)ethyl)uridine-3' -phosphate
  misc_feature 10..20: note = RNA
  misc_feature 1..9: note = DNA
tttttttttttttttttttt

SEQ ID NO: 5 - DNA, 20 bp, synthetic construct
  source 1..20: mol_type = other DNA
  modified_base 10: mod_base = OTHER; note = 2'-O-(2-oxo-2-(N-octadec-1-ylamino)ethyl)uridine-3' -phosphate
  misc_feature 10..20: note = RNA
  misc_feature 1..9: note = DNA
tttttttttttttttttttt

SEQ ID NO: 6 - DNA, 20 bp, synthetic construct
  source 1..20: mol_type = other DNA
  modified_base 10: mod_base = OTHER; note = 2'-O-(2-oxo-2-(N-octadec-1-ylamino)ethyl)cytidine-3 '-phosphate
  misc_feature 10..20: note = RNA
  misc_feature 1..9: note = DNA
tttttttttctttttttttt

SEQ ID NO: 7 - DNA, 20 bp, synthetic construct
  source 1..20: mol_type = other DNA
  modified_base 10: mod_base = OTHER; note = 2'-O-(2-oxo-2-(N-tetradec-1-ylamino)ethyl)cytidine- 3'-phosphate
  misc_feature 10..20: note = RNA
  misc_feature 1..9: note = DNA
tttttttttctttttttttt

SEQ ID NO: 8 - RNA, 21 nt, synthetic construct
  source 1..21: mol_type = other RNA
cattttaatcctcactctaaa

SEQ ID NO: 9 - RNA, 23 nt, synthetic construct
  source 1..23: mol_type = other RNA
tttagagtgaggattaaaatgag

SEQ ID NO: 10 - RNA, 21 nt, synthetic construct
  source 1..21: mol_type = other RNA
  modified_base 1: mod_base = OTHER; note = 2'-O-methylcytidine-3'-phosphorothioate
  modified_base 2: mod_base = OTHER; note = 2'-O-methyladenosine-3'-phosphorothioate
  modified_base 3: mod_base = OTHER; note = 2'-O-methyluridine-3'-phosphate
  modified_base 4: mod_base = OTHER; note = 2'-O-methyluridine-3'-phosphate
  modified_base 5: mod_base = OTHER; note = 2'-O-methyluridine-3'-phosphate
  modified_base 6: mod_base = OTHER; note = 2'-O-hexadecyl-uridine-3'-phosphate
  modified_base 7: mod_base = OTHER; note = 2'-deoxy-2'-fluoroadenosine-3'-phosphate
  modified_base 8: mod_base = OTHER; note = 2'-O-methyladenosine-3'-phosphate
  modified_base 9: mod_base = OTHER; note = 2'-deoxy-2'-fluorouridine-3'-phosphate
  modified_base 10: mod_base = OTHER; note = 2'-deoxy-2'-fluorocytidine-3'-phosphate
  modified_base 11: mod_base = OTHER; note = 2'-deoxy-2'-fluorocytidine-3'-phosphate
  modified_base 12: mod_base = OTHER; note = 2'-O-methyluridine-3'-phosphate
  modified_base 13: mod_base = OTHER; note = 2'-O-methylcytidine-3'-phosphate
  modified_base 14: mod_base = OTHER; note = 2'-O-methyladenosine-3'-phosphate
  modified_base 15: mod_base = OTHER; note = 2'-O-methylcytidine-3'-phosphate
  modified_base 16: mod_base = OTHER; note = 2'-O-methyluridine-3'-phosphate
  modified_base 17: mod_base = OTHER; note = 2'-O-methylcytidine-3'-phosphate
  modified_base 18: mod_base = OTHER; note = 2'-O-methyluridine-3'-phosphate
  modified_base 19: mod_base = OTHER; note = 2'-O-methyladenosine-3'-phosphorothioate
  modified_base 20: mod_base = OTHER; note = 2'-O-methyladenosine-3'-phosphorothioate
  modified_base 21: mod_base = OTHER; note = 2'-O-methyladenosine
cattttaatcctcactctaaa

SEQ ID NO: 11 - RNA, 23 nt, synthetic construct
  source 1..23: mol_type = other RNA
  modified_base 1: mod_base = OTHER; note = 2'-O-methyluridine-3'-phosphorothioate 5'-(E)-Vinylphosphonate
  modified_base 2: mod_base = OTHER; note = 2'-deoxy-2'-fluorouridine-3'-phosphorothioate
  modified_base 3: mod_base = OTHER; note = 2'-O-methyluridine-3'-phosphate
  modified_base 4: mod_base = OTHER; note = 2'-O-methyladenosine-3'-phosphate
  modified_base 5: mod_base = OTHER; note = 2'-O-methylguanosine-3'-phosphate
  modified_base 6: mod_base = OTHER; note = 2'-deoxy-2'-fluoroadenosine-3'-phosphate
  modified_base 7: mod_base = OTHER; note = 2'-O-methylguanosine-3'-phosphate
  modified_base 8: mod_base = OTHER; note = 2'-deoxy-2'-fluorouridine-3'-phosphate
  modified_base 9: mod_base = OTHER; note = 2'-deoxy-2'-fluoroguanosine-3'-phosphate
  modified_base 10: mod_base = OTHER; note = 2'-O-methyladenosine-3'-phosphate
  modified_base 11: mod_base = OTHER; note = 2'-O-methylguanosine-3'-phosphate
  modified_base 12: mod_base = OTHER; note = 2'-O-methylguanosine-3'-phosphate
  modified_base 13: mod_base = OTHER; note = 2'-O-methyladenosine-3'-phosphate
  modified_base 14: mod_base = OTHER; note = 2'-deoxy-2'-fluorouridine-3'-phosphate
  modified_base 15: mod_base = OTHER; note = 2'-O-methyluridine-3'-phosphate
  modified_base 16: mod_base = OTHER; note = 2'-deoxy-2'-fluoroadenosine-3'-phosphate
  modified_base 17: mod_base = OTHER; note = 2'-O-methyladenosine-3'-phosphate
  modified_base 18: mod_base = OTHER; note = 2'-O-methyladenosine-3'-phosphate
  modified_base 19: mod_base = OTHER; note = 2'-O-methyladenosine-3'-phosphate
  modified_base 20: mod_base = OTHER; note = 2'-O-methyluridine-3'-phosphate
  modified_base 21: mod_base = OTHER; note = 2'-O-methylguanosine-3'-phosphorothioate
  modified_base 22: mod_base = OTHER; note = 2'-O-methyladenosine-3'-phosphorothioate
  modified_base 23: mod_base = OTHER; note = 2'-O-methylguanosine
tttagagtgaggattaaaatgag

SEQ ID NO: 12 - RNA, 21 nt, synthetic construct
  source 1..21: mol_type = other RNA
  modified_base 1: mod_base = OTHER; note = 2'-O-methylcytidine-3'-phosphorothioate
  modified_base 2: mod_base = OTHER; note = 2'-O-methyladenosine-3'-phosphorothioate
  modified_base 3: mod_base = OTHER; note = 2'-O-methyluridine-3'-phosphate
  modified_base 4: mod_base = OTHER; note = 2'-O-methyluridine-3'-phosphate
  modified_base 5: mod_base = OTHER; note = 2'-O-methyluridine-3'-phosphate
  modified_base 6: mod_base = OTHER; note = 3'-O-(2-(1-hexadecyloxy)ethyl)uridine-2'-phosphate
  modified_base 7: mod_base = OTHER; note = 2'-deoxy-2'-fluoroadenosine-3'-phosphate
  modified_base 8: mod_base = OTHER; note = 2'-O-methyladenosine-3'-phosphate
  modified_base 9: mod_base = OTHER; note = 2'-deoxy-2'-fluorouridine-3'-phosphate
  modified_base 10: mod_base = OTHER; note = 2'-deoxy-2'-fluorocytidine-3'-phosphate
  modified_base 11: mod_base = OTHER; note = 2'-deoxy-2'-fluorocytidine-3'-phosphate
  modified_base 12: mod_base = OTHER; note = 2'-O-methyluridine-3'-phosphate
  modified_base 13: mod_base = OTHER; note = 2'-O-methylcytidine-3'-phosphate
  modified_base 14: mod_base = OTHER; note = 2'-O-methyladenosine-3'-phosphate
  modified_base 15: mod_base = OTHER; note = 2'-O-methylcytidine-3'-phosphate
  modified_base 16: mod_base = OTHER; note = 2'-O-methyluridine-3'-phosphate
  modified_base 17: mod_base = OTHER; note = 2'-O-methylcytidine-3'-phosphate
  modified_base 18: mod_base = OTHER; note = 2'-O-methyluridine-3'-phosphate
  modified_base 19: mod_base = OTHER; note = 2'-O-methyladenosine-3'-phosphorothioate
  modified_base 20: mod_base = OTHER; note = 2'-O-methyladenosine-3'-phosphorothioate
  modified_base 21: mod_base = OTHER; note = 2'-O-methyladenosine
cattttaatcctcactctaaa

SEQ ID NO: 13 - RNA, 23 nt, synthetic construct
  source 1..23: mol_type = other RNA
  modified_base 1: mod_base = OTHER; note = 2'-O-methyluridine-3'-phosphorothioate 5'-(E)-Vinylphosphonate
  modified_base 2: mod_base = OTHER; note = 2'-deoxy-2'-fluorouridine-3'-phosphorothioate
  modified_base 3: mod_base = OTHER; note = 2'-O-methyluridine-3'-phosphate
  modified_base 4: mod_base = OTHER; note = 2'-O-methyladenosine-3'-phosphate
  modified_base 5: mod_base = OTHER; note = 2'-O-methylguanosine-3'-phosphate
  modified_base 6: mod_base = OTHER; note = 2'-deoxy-2'-fluoroadenosine-3'-phosphate
  modified_base 7: mod_base = OTHER; note = 2'-O-methylguanosine-3'-phosphate
  modified_base 8: mod_base = OTHER; note = 2'-deoxy-2'-fluorouridine-3'-phosphate
  modified_base 9: mod_base = OTHER; note = 2'-deoxy-2'-fluoroguanosine-3'-phosphate
  modified_base 10: mod_base = OTHER; note = 2'-O-methyladenosine-3'-phosphate
  modified_base 11: mod_base = OTHER; note = 2'-O-methylguanosine-3'-phosphate
  modified_base 12: mod_base = OTHER; note = 2'-O-methylguanosine-3'-phosphate
  modified_base 13: mod_base = OTHER; note = 2'-O-methyladenosine-3'-phosphate
  modified_base 14: mod_base = OTHER; note = 2'-deoxy-2'-fluorouridine-3'-phosphate
  modified_base 15: mod_base = OTHER; note = 2'-O-methyluridine-3'-phosphate
  modified_base 16: mod_base = OTHER; note = 2'-deoxy-2'-fluoroadenosine-3'-phosphate
  modified_base 17: mod_base = OTHER; note = 2'-O-methyladenosine-3'-phosphate
  modified_base 18: mod_base = OTHER; note = 2'-O-methyladenosine-3'-phosphate
  modified_base 19: mod_base = OTHER; note = 2'-O-methyladenosine-3'-phosphate
  modified_base 20: mod_base = OTHER; note = 2'-O-methyluridine-3'-phosphate
  modified_base 21: mod_base = OTHER; note = 2'-O-methylguanosine-3'-phosphorothioate
  modified_base 22: mod_base = OTHER; note = 2'-O-methyladenosine-3'-phosphorothioate
  modified_base 23: mod_base = OTHER; note = 2'-O-methylguanosine
tttagagtgaggattaaaatgag

SEQ ID NO: 14 - RNA, 21 nt, synthetic construct
  source 1..21: mol_type = other RNA
  modified_base 1: mod_base = OTHER; note = 2'-O-methylcytidine-3'-phosphorothioate
  modified_base 2: mod_base = OTHER; note = 2'-O-methyladenosine-3'-phosphorothioate
  modified_base 3: mod_base = OTHER; note = 2'-O-methyluridine-3'-phosphate
  modified_base 4: mod_base = OTHER; note = 2'-O-methyluridine-3'-phosphate
  modified_base 5: mod_base = OTHER; note = 2'-O-methyluridine-3'-phosphate
  modified_base 6: mod_base = OTHER; note = 2'-O-(2-(1-hexadecyloxy)ethyl)uridine-3'-phosphate
  modified_base 7: mod_base = OTHER; note = 2'-deoxy-2'-fluoroadenosine-3'-phosphate
  modified_base 8: mod_base = OTHER; note = 2'-O-methyladenosine-3'-phosphate
  modified_base 9: mod_base = OTHER; note = 2'-deoxy-2'-fluorouridine-3'-phosphate
  modified_base 10: mod_base = OTHER; note = 2'-deoxy-2'-fluorocytidine-3'-phosphate
  modified_base 11: mod_base = OTHER; note = 2'-deoxy-2'-fluorocytidine-3'-phosphate
  modified_base 12: mod_base = OTHER; note = 2'-O-methyluridine-3'-phosphate
  modified_base 13: mod_base = OTHER; note = 2'-O-methylcytidine-3'-phosphate
  modified_base 14: mod_base = OTHER; note = 2'-O-methyladenosine-3'-phosphate
  modified_base 15: mod_base = OTHER; note = 2'-O-methylcytidine-3'-phosphate
  modified_base 16: mod_base = OTHER; note = 2'-O-methyluridine-3'-phosphate
  modified_base 17: mod_base = OTHER; note = 2'-O-methylcytidine-3'-phosphate
  modified_base 18: mod_base = OTHER; note = 2'-O-methyluridine-3'-phosphate
  modified_base 19: mod_base = OTHER; note = 2'-O-methyladenosine-3'-phosphorothioate
  modified_base 20: mod_base = OTHER; note = 2'-O-methyladenosine-3'-phosphorothioate
  modified_base 21: mod_base = OTHER; note = 2'-O-methyladenosine
cattttaatcctcactctaaa

SEQ ID NO: 15 - RNA, 23 nt, synthetic construct
  source 1..23: mol_type = other RNA
  modified_base 1: mod_base = OTHER; note = 2'-O-methyluridine-3'-phosphorothioate 5'-(E)-Vinylphosphonate
  modified_base 2: mod_base = OTHER; note = 2'-deoxy-2'-fluorouridine-3'-phosphorothioate
  modified_base 3: mod_base = OTHER; note = 2'-O-methyluridine-3'-phosphate
  modified_base 4: mod_base = OTHER; note = 2'-O-methyladenosine-3'-phosphate
  modified_base 5: mod_base = OTHER; note = 2'-O-methylguanosine-3'-phosphate
  modified_base 6: mod_base = OTHER; note = 2'-deoxy-2'-fluoroadenosine-3'-phosphate
  modified_base 7: mod_base = OTHER; note = 2'-O-methylguanosine-3'-phosphate
  modified_base 8: mod_base = OTHER; note = 2'-deoxy-2'-fluorouridine-3'-phosphate
  modified_base 9: mod_base = OTHER; note = 2'-deoxy-2'-fluoroguanosine-3'-phosphate
  modified_base 10: mod_base = OTHER; note = 2'-O-methyladenosine-3'-phosphate
  modified_base 11: mod_base = OTHER; note = 2'-O-methylguanosine-3'-phosphate
  modified_base 12: mod_base = OTHER; note = 2'-O-methylguanosine-3'-phosphate
  modified_base 13: mod_base = OTHER; note = 2'-O-methyladenosine-3'-phosphate
  modified_base 14: mod_base = OTHER; note = 2'-deoxy-2'-fluorouridine-3'-phosphate
  modified_base 15: mod_base = OTHER; note = 2'-O-methyluridine-3'-phosphate
  modified_base 16: mod_base = OTHER; note = 2'-deoxy-2'-fluoroadenosine-3'-phosphate
  modified_base 17: mod_base = OTHER; note = 2'-O-methyladenosine-3'-phosphate
  modified_base 18: mod_base = OTHER; note = 2'-O-methyladenosine-3'-phosphate
  modified_base 19: mod_base = OTHER; note = 2'-O-methyladenosine-3'-phosphate
  modified_base 20: mod_base = OTHER; note = 2'-O-methyluridine-3'-phosphate
  modified_base 21: mod_base = OTHER; note = 2'-O-methylguanosine-3'-phosphorothioate
  modified_base 22: mod_base = OTHER; note = 2'-O-methyladenosine-3'-phosphorothioate
  modified_base 23: mod_base = OTHER; note = 2'-O-methylguanosine
tttagagtgaggattaaaatgag

SEQ ID NO: 16 - RNA, 21 nt, synthetic construct
  source 1..21: mol_type = other RNA
  modified_base 1: mod_base = OTHER; note = 2'-O-methylcytidine-3'-phosphorothioate
  modified_base 2: mod_base = OTHER; note = 2'-O-methyladenosine-3'-phosphorothioate
  modified_base 3: mod_base = OTHER; note = 2'-O-methyluridine-3'-phosphate
  modified_base 4: mod_base = OTHER; note = 2'-O-methyluridine-3'-phosphate
  modified_base 5: mod_base = OTHER; note = 2'-O-methyluridine-3'-phosphate
  modified_base 6: mod_base = OTHER; note = 2'-O-(2-(1-octadecyloxy)ethyl)uridine-3'-phosphate
  modified_base 7: mod_base = OTHER; note = 2'-deoxy-2'-fluoroadenosine-3'-phosphate
  modified_base 8: mod_base = OTHER; note = 2'-O-methyladenosine-3'-phosphate
  modified_base 9: mod_base = OTHER; note = 2'-deoxy-2'-fluorouridine-3'-phosphate
  modified_base 10: mod_base = OTHER; note = 2'-deoxy-2'-fluorocytidine-3'-phosphate
  modified_base 11: mod_base = OTHER; note = 2'-deoxy-2'-fluorocytidine-3'-phosphate
  modified_base 12: mod_base = OTHER; note = 2'-O-methyluridine-3'-phosphate
  modified_base 13: mod_base = OTHER; note = 2'-O-methylcytidine-3'-phosphate
  modified_base 14: mod_base = OTHER; note = 2'-O-methyladenosine-3'-phosphate
  modified_base 15: mod_base = OTHER; note = 2'-O-methylcytidine-3'-phosphate
  modified_base 16: mod_base = OTHER; note = 2'-O-methyluridine-3'-phosphate
  modified_base 17: mod_base = OTHER; note = 2'-O-methylcytidine-3'-phosphate
  modified_base 18: mod_base = OTHER; note = 2'-O-methyluridine-3'-phosphate
  modified_base 19: mod_base = OTHER; note = 2'-O-methyladenosine-3'-phosphorothioate
  modified_base 20: mod_base = OTHER; note = 2'-O-methyladenosine-3'-phosphorothioate
  modified_base 21: mod_base = OTHER; note = 2'-O-methyladenosine
cattttaatcctcactctaaa

SEQ ID NO: 17 - RNA, 23 nt, synthetic construct
  source 1..23: mol_type = other RNA
  modified_base 1: mod_base = OTHER; note = 2'-O-methyluridine-3'-phosphorothioate 5'-(E)-Vinylphosphonate
  modified_base 2: mod_base = OTHER; note = 2'-deoxy-2'-fluorouridine-3'-phosphorothioate
  modified_base 3: mod_base = OTHER; note = 2'-O-methyluridine-3'-phosphate
  modified_base 4: mod_base = OTHER; note = 2'-O-methyladenosine-3'-phosphate
  modified_base 5: mod_base = OTHER; note = 2'-O-methylguanosine-3'-phosphate
  modified_base 6: mod_base = OTHER; note = 2'-deoxy-2'-fluoroadenosine-3'-phosphate
  modified_base 7: mod_base = OTHER; note = 2'-O-methylguanosine-3'-phosphate
  modified_base 8: mod_base = OTHER; note = 2'-deoxy-2'-fluorouridine-3'-phosphate
  modified_base 9: mod_base = OTHER; note = 2'-deoxy-2'-fluoroguanosine-3'-phosphate
  modified_base 10: mod_base = OTHER; note = 2'-O-methyladenosine-3'-phosphate
  modified_base 11: mod_base = OTHER; note = 2'-O-methylguanosine-3'-phosphate
  modified_base 12: mod_base = OTHER; note = 2'-O-methylguanosine-3'-phosphate
  modified_base 13: mod_base = OTHER; note = 2'-O-methyladenosine-3'-phosphate
  modified_base 14: mod_base = OTHER; note = 2'-deoxy-2'-fluorouridine-3'-phosphate
  modified_base 15: mod_base = OTHER; note = 2'-O-methyluridine-3'-phosphate
  modified_base 16: mod_base = OTHER; note = 2'-deoxy-2'-fluoroadenosine-3'-phosphate
  modified_base 17: mod_base = OTHER; note = 2'-O-methyladenosine-3'-phosphate
  modified_base 18: mod_base = OTHER; note = 2'-O-methyladenosine-3'-phosphate
  modified_base 19: mod_base = OTHER; note = 2'-O-methyladenosine-3'-phosphate
  modified_base 20: mod_base = OTHER; note = 2'-O-methyluridine-3'-phosphate
  modified_base 21: mod_base = OTHER; note = 2'-O-methylguanosine-3'-phosphorothioate
  modified_base 22: mod_base = OTHER; note = 2'-O-methyladenosine-3'-phosphorothioate
  modified_base 23: mod_base = OTHER; note = 2'-O-methylguanosine
tttagagtgaggattaaaatgag

SEQ ID NO: 18 - RNA, 21 nt, synthetic construct
  source 1..21: mol_type = other RNA
  modified_base 1: mod_base = OTHER; note = 2'-O-methylcytidine-3'-phosphorothioate
  modified_base 2: mod_base = OTHER; note = 2'-O-methyladenosine-3'-phosphorothioate
  modified_base 3: mod_base = OTHER; note = 2'-O-methyluridine-3'-phosphate
  modified_base 4: mod_base = OTHER; note = 2'-O-methyluridine-3'-phosphate
  modified_base 5: mod_base = OTHER; note = 2'-O-methyluridine-3'-phosphate
  modified_base 6: mod_base = OTHER; note = 2'-O-(2-(1-octadec-9Z-enoxy)ethyl)uridine-3'-phosph ate
  modified_base 7: mod_base = OTHER; note = 2'-deoxy-2'-fluoroadenosine-3'-phosphate
  modified_base 8: mod_base = OTHER; note = 2'-O-methyladenosine-3'-phosphate
  modified_base 9: mod_base = OTHER; note = 2'-deoxy-2'-fluorouridine-3'-phosphate
  modified_base 10: mod_base = OTHER; note = 2'-deoxy-2'-fluorocytidine-3'-phosphate
  modified_base 11: mod_base = OTHER; note = 2'-deoxy-2'-fluorocytidine-3'-phosphate
  modified_base 12: mod_base = OTHER; note = 2'-O-methyluridine-3'-phosphate
  modified_base 13: mod_base = OTHER; note = 2'-O-methylcytidine-3'-phosphate
  modified_base 14: mod_base = OTHER; note = 2'-O-methyladenosine-3'-phosphate
  modified_base 15: mod_base = OTHER; note = 2'-O-methylcytidine-3'-phosphate
  modified_base 16: mod_base = OTHER; note = 2'-O-methyluridine-3'-phosphate
  modified_base 17: mod_base = OTHER; note = 2'-O-methylcytidine-3'-phosphate
  modified_base 18: mod_base = OTHER; note = 2'-O-methyluridine-3'-phosphate
  modified_base 19: mod_base = OTHER; note = 2'-O-methyladenosine-3'-phosphorothioate
  modified_base 20: mod_base = OTHER; note = 2'-O-methyladenosine-3'-phosphorothioate
  modified_base 21: mod_base = OTHER; note = 2'-O-methyladenosine
cattttaatcctcactctaaa

SEQ ID NO: 19 - RNA, 23 nt, synthetic construct
  source 1..23: mol_type = other RNA
  modified_base 1: mod_base = OTHER; note = 2'-O-methyluridine-3'-phosphorothioate 5'-(E)-Vinylphosphonate
  modified_base 2: mod_base = OTHER; note = 2'-deoxy-2'-fluorouridine-3'-phosphorothioate
  modified_base 3: mod_base = OTHER; note = 2'-O-methyluridine-3'-phosphate
  modified_base 4: mod_base = OTHER; note = 2'-O-methyladenosine-3'-phosphate
  modified_base 5: mod_base = OTHER; note = 2'-O-methylguanosine-3'-phosphate
  modified_base 6: mod_base = OTHER; note = 2'-deoxy-2'-fluoroadenosine-3'-phosphate
  modified_base 7: mod_base = OTHER; note = 2'-O-methylguanosine-3'-phosphate
  modified_base 8: mod_base = OTHER; note = 2'-deoxy-2'-fluorouridine-3'-phosphate
  modified_base 9: mod_base = OTHER; note = 2'-deoxy-2'-fluoroguanosine-3'-phosphate
  modified_base 10: mod_base = OTHER; note = 2'-O-methyladenosine-3'-phosphate
  modified_base 11: mod_base = OTHER; note = 2'-O-methylguanosine-3'-phosphate
  modified_base 12: mod_base = OTHER; note = 2'-O-methylguanosine-3'-phosphate
  modified_base 13: mod_base = OTHER; note = 2'-O-methyladenosine-3'-phosphate
  modified_base 14: mod_base = OTHER; note = 2'-deoxy-2'-fluorouridine-3'-phosphate
  modified_base 15: mod_base = OTHER; note = 2'-O-methyluridine-3'-phosphate
  modified_base 16: mod_base = OTHER; note = 2'-deoxy-2'-fluoroadenosine-3'-phosphate
  modified_base 17: mod_base = OTHER; note = 2'-O-methyladenosine-3'-phosphate
  modified_base 18: mod_base = OTHER; note = 2'-O-methyladenosine-3'-phosphate
  modified_base 19: mod_base = OTHER; note = 2'-O-methyladenosine-3'-phosphate
  modified_base 20: mod_base = OTHER; note = 2'-O-methyluridine-3'-phosphate
  modified_base 21: mod_base = OTHER; note = 2'-O-methylguanosine-3'-phosphorothioate
  modified_base 22: mod_base = OTHER; note = 2'-O-methyladenosine-3'-phosphorothioate
  modified_base 23: mod_base = OTHER; note = 2'-O-methylguanosine
tttagagtgaggattaaaatgag

SEQ ID NO: 20 - RNA, 21 nt, synthetic construct
  source 1..21: mol_type = other RNA
  modified_base 1: mod_base = OTHER; note = 2'-O-methylcytidine-3'-phosphorothioate
  modified_base 2: mod_base = OTHER; note = 2'-O-methyladenosine-3'-phosphorothioate
  modified_base 3: mod_base = OTHER; note = 2'-O-methyluridine-3'-phosphate
  modified_base 4: mod_base = OTHER; note = 2'-O-methyluridine-3'-phosphate
  modified_base 5: mod_base = OTHER; note = 2'-O-methyluridine-3'-phosphate
  modified_base 6: mod_base = OTHER; note = 2'-O-(2-oxo-2-(N-heptadec-9-ylamino)ethyl)uridine-3 '-phosphate
  modified_base 7: mod_base = OTHER; note = 2'-deoxy-2'-fluoroadenosine-3'-phosphate
  modified_base 8: mod_base = OTHER; note = 2'-O-methyladenosine-3'-phosphate
  modified_base 9: mod_base = OTHER; note = 2'-deoxy-2'-fluorouridine-3'-phosphate
  modified_base 10: mod_base = OTHER; note = 2'-deoxy-2'-fluorocytidine-3'-phosphate
  modified_base 11: mod_base = OTHER; note = 2'-deoxy-2'-fluorocytidine-3'-phosphate
  modified_base 12: mod_base = OTHER; note = 2'-O-methyluridine-3'-phosphate
  modified_base 13: mod_base = OTHER; note = 2'-O-methylcytidine-3'-phosphate
  modified_base 14: mod_base = OTHER; note = 2'-O-methyladenosine-3'-phosphate
  modified_base 15: mod_base = OTHER; note = 2'-O-methylcytidine-3'-phosphate
  modified_base 16: mod_base = OTHER; note = 2'-O-methyluridine-3'-phosphate
  modified_base 17: mod_base = OTHER; note = 2'-O-methylcytidine-3'-phosphate
  modified_base 18: mod_base = OTHER; note = 2'-O-methyluridine-3'-phosphate
  modified_base 19: mod_base = OTHER; note = 2'-O-methyladenosine-3'-phosphorothioate
  modified_base 20: mod_base = OTHER; note = 2'-O-methyladenosine-3'-phosphorothioate
  modified_base 21: mod_base = OTHER; note = 2'-O-methyladenosine
cattttaatcctcactctaaa

SEQ ID NO: 21 - RNA, 23 nt, synthetic construct
  source 1..23: mol_type = other RNA
  modified_base 1: mod_base = OTHER; note = 2'-O-methyluridine-3'-phosphorothioate 5'-(E)-Vinylphosphonate
  modified_base 2: mod_base = OTHER; note = 2'-deoxy-2'-fluorouridine-3'-phosphorothioate
  modified_base 3: mod_base = OTHER; note = 2'-O-methyluridine-3'-phosphate
  modified_base 4: mod_base = OTHER; note = 2'-O-methyladenosine-3'-phosphate
  modified_base 5: mod_base = OTHER; note = 2'-O-methylguanosine-3'-phosphate
  modified_base 6: mod_base = OTHER; note = 2'-deoxy-2'-fluoroadenosine-3'-phosphate
  modified_base 7: mod_base = OTHER; note = 2'-O-methylguanosine-3'-phosphate
  modified_base 8: mod_base = OTHER; note = 2'-deoxy-2'-fluorouridine-3'-phosphate
  modified_base 9: mod_base = OTHER; note = 2'-deoxy-2'-fluoroguanosine-3'-phosphate
  modified_base 10: mod_base = OTHER; note = 2'-O-methyladenosine-3'-phosphate
  modified_base 11: mod_base = OTHER; note = 2'-O-methylguanosine-3'-phosphate
  modified_base 12: mod_base = OTHER; note = 2'-O-methylguanosine-3'-phosphate
  modified_base 13: mod_base = OTHER; note = 2'-O-methyladenosine-3'-phosphate
  modified_base 14: mod_base = OTHER; note = 2'-deoxy-2'-fluorouridine-3'-phosphate
  modified_base 15: mod_base = OTHER; note = 2'-O-methyluridine-3'-phosphate
  modified_base 16: mod_base = OTHER; note = 2'-deoxy-2'-fluoroadenosine-3'-phosphate
  modified_base 17: mod_base = OTHER; note = 2'-O-methyladenosine-3'-phosphate
  modified_base 18: mod_base = OTHER; note = 2'-O-methyladenosine-3'-phosphate
  modified_base 19: mod_base = OTHER; note = 2'-O-methyladenosine-3'-phosphate
  modified_base 20: mod_base = OTHER; note = 2'-O-methyluridine-3'-phosphate
  modified_base 21: mod_base = OTHER; note = 2'-O-methylguanosine-3'-phosphorothioate
  modified_base 22: mod_base = OTHER; note = 2'-O-methyladenosine-3'-phosphorothioate
  modified_base 23: mod_base = OTHER; note = 2'-O-methylguanosine
tttagagtgaggattaaaatgag

SEQ ID NO: 22 - RNA, 21 nt, synthetic construct
  source 1..21: mol_type = other RNA
  modified_base 1: mod_base = OTHER; note = 2'-O-methylcytidine-3'-phosphorothioate
  modified_base 2: mod_base = OTHER; note = 2'-O-methyladenosine-3'-phosphorothioate
  modified_base 3: mod_base = OTHER; note = 2'-O-methyluridine-3'-phosphate
  modified_base 4: mod_base = OTHER; note = 2'-O-methyluridine-3'-phosphate
  modified_base 5: mod_base = OTHER; note = 2'-O-methyluridine-3'-phosphate
  modified_base 6: mod_base = OTHER; note = 2'-O-(2-oxo-2-(N,N-didecan-1-ylamino)ethyl)-uridine -3'-phosphate
  modified_base 7: mod_base = OTHER; note = 2'-deoxy-2'-fluoroadenosine-3'-phosphate
  modified_base 8: mod_base = OTHER; note = 2'-O-methyladenosine-3'-phosphate
  modified_base 9: mod_base = OTHER; note = 2'-deoxy-2'-fluorouridine-3'-phosphate
  modified_base 10: mod_base = OTHER; note = 2'-deoxy-2'-fluorocytidine-3'-phosphate
  modified_base 11: mod_base = OTHER; note = 2'-deoxy-2'-fluorocytidine-3'-phosphate
  modified_base 12: mod_base = OTHER; note = 2'-O-methyluridine-3'-phosphate
  modified_base 13: mod_base = OTHER; note = 2'-O-methylcytidine-3'-phosphate
  modified_base 14: mod_base = OTHER; note = 2'-O-methyladenosine-3'-phosphate
  modified_base 15: mod_base = OTHER; note = 2'-O-methylcytidine-3'-phosphate
  modified_base 16: mod_base = OTHER; note = 2'-O-methyluridine-3'-phosphate
  modified_base 17: mod_base = OTHER; note = 2'-O-methylcytidine-3'-phosphate
  modified_base 18: mod_base = OTHER; note = 2'-O-methyluridine-3'-phosphate
  modified_base 19: mod_base = OTHER; note = 2'-O-methyladenosine-3'-phosphorothioate
  modified_base 20: mod_base = OTHER; note = 2'-O-methyladenosine-3'-phosphorothioate
  modified_base 21: mod_base = OTHER; note = 2'-O-methyladenosine
cattttaatcctcactctaaa

SEQ ID NO: 23 - RNA, 23 nt, synthetic construct
  source 1..23: mol_type = other RNA
  modified_base 1: mod_base = OTHER; note = 2'-O-methyluridine-3'-phosphorothioate 5'-(E)-Vinylphosphonate
  modified_base 2: mod_base = OTHER; note = 2'-deoxy-2'-fluorouridine-3'-phosphorothioate
  modified_base 3: mod_base = OTHER; note = 2'-O-methyluridine-3'-phosphate
  modified_base 4: mod_base = OTHER; note = 2'-O-methyladenosine-3'-phosphate
  modified_base 5: mod_base = OTHER; note = 2'-O-methylguanosine-3'-phosphate
  modified_base 6: mod_base = OTHER; note = 2'-deoxy-2'-fluoroadenosine-3'-phosphate
  modified_base 7: mod_base = OTHER; note = 2'-O-methylguanosine-3'-phosphate
  modified_base 8: mod_base = OTHER; note = 2'-deoxy-2'-fluorouridine-3'-phosphate
  modified_base 9: mod_base = OTHER; note = 2'-deoxy-2'-fluoroguanosine-3'-phosphate
  modified_base 10: mod_base = OTHER; note = 2'-O-methyladenosine-3'-phosphate
  modified_base 11: mod_base = OTHER; note = 2'-O-methylguanosine-3'-phosphate
  modified_base 12: mod_base = OTHER; note = 2'-O-methylguanosine-3'-phosphate
  modified_base 13: mod_base = OTHER; note = 2'-O-methyladenosine-3'-phosphate
  modified_base 14: mod_base = OTHER; note = 2'-deoxy-2'-fluorouridine-3'-phosphate
  modified_base 15: mod_base = OTHER; note = 2'-O-methyluridine-3'-phosphate
  modified_base 16: mod_base = OTHER; note = 2'-deoxy-2'-fluoroadenosine-3'-phosphate
  modified_base 17: mod_base = OTHER; note = 2'-O-methyladenosine-3'-phosphate
  modified_base 18: mod_base = OTHER; note = 2'-O-methyladenosine-3'-phosphate
  modified_base 19: mod_base = OTHER; note = 2'-O-methyladenosine-3'-phosphate
  modified_base 20: mod_base = OTHER; note = 2'-O-methyluridine-3'-phosphate
  modified_base 21: mod_base = OTHER; note = 2'-O-methylguanosine-3'-phosphorothioate
  modified_base 22: mod_base = OTHER; note = 2'-O-methyladenosine-3'-phosphorothioate
  modified_base 23: mod_base = OTHER; note = 2'-O-methylguanosine
tttagagtgaggattaaaatgag

SEQ ID NO: 24 - RNA, 21 nt, synthetic construct
  source 1..21: mol_type = other RNA
  modified_base 1: mod_base = OTHER; note = 2'-O-methylcytidine-3'-phosphorothioate
  modified_base 2: mod_base = OTHER; note = 2'-O-methyladenosine-3'-phosphorothioate
  modified_base 3: mod_base = OTHER; note = 2'-O-methyluridine-3'-phosphate
  modified_base 4: mod_base = OTHER; note = 2'-O-methyluridine-3'-phosphate
  modified_base 5: mod_base = OTHER; note = 2'-O-methyluridine-3'-phosphate
  modified_base 6: mod_base = OTHER; note = 2'-O-(2-oxo-2-(N,N-dioctan-1-ylamino)ethyl)-uridine -3'-phosphate
  modified_base 7: mod_base = OTHER; note = 2'-deoxy-2'-fluoroadenosine-3'-phosphate
  modified_base 8: mod_base = OTHER; note = 2'-O-methyladenosine-3'-phosphate
  modified_base 9: mod_base = OTHER; note = 2'-deoxy-2'-fluorouridine-3'-phosphate
  modified_base 10: mod_base = OTHER; note = 2'-deoxy-2'-fluorocytidine-3'-phosphate
  modified_base 11: mod_base = OTHER; note = 2'-deoxy-2'-fluorocytidine-3'-phosphate
  modified_base 12: mod_base = OTHER; note = 2'-O-methyluridine-3'-phosphate
  modified_base 13: mod_base = OTHER; note = 2'-O-methylcytidine-3'-phosphate
  modified_base 14: mod_base = OTHER; note = 2'-O-methyladenosine-3'-phosphate
  modified_base 15: mod_base = OTHER; note = 2'-O-methylcytidine-3'-phosphate
  modified_base 16: mod_base = OTHER; note = 2'-O-methyluridine-3'-phosphate
  modified_base 17: mod_base = OTHER; note = 2'-O-methylcytidine-3'-phosphate
  modified_base 18: mod_base = OTHER; note = 2'-O-methyluridine-3'-phosphate
  modified_base 19: mod_base = OTHER; note = 2'-O-methyladenosine-3'-phosphorothioate
  modified_base 20: mod_base = OTHER; note = 2'-O-methyladenosine-3'-phosphorothioate
  modified_base 21: mod_base = OTHER; note = 2'-O-methyladenosine
cattttaatcctcactctaaa

SEQ ID NO: 25 - RNA, 23 nt, synthetic construct
  source 1..23: mol_type = other RNA
  modified_base 1: mod_base = OTHER; note = 2'-O-methyluridine-3'-phosphorothioate 5'-(E)-Vinylphosphonate
  modified_base 2: mod_base = OTHER; note = 2'-deoxy-2'-fluorouridine-3'-phosphorothioate
  modified_base 3: mod_base = OTHER; note = 2'-O-methyluridine-3'-phosphate
  modified_base 4: mod_base = OTHER; note = 2'-O-methyladenosine-3'-phosphate
  modified_base 5: mod_base = OTHER; note = 2'-O-methylguanosine-3'-phosphate
  modified_base 6: mod_base = OTHER; note = 2'-deoxy-2'-fluoroadenosine-3'-phosphate
  modified_base 7: mod_base = OTHER; note = 2'-O-methylguanosine-3'-phosphate
  modified_base 8: mod_base = OTHER; note = 2'-deoxy-2'-fluorouridine-3'-phosphate
  modified_base 9: mod_base = OTHER; note = 2'-deoxy-2'-fluoroguanosine-3'-phosphate
  modified_base 10: mod_base = OTHER; note = 2'-O-methyladenosine-3'-phosphate
  modified_base 11: mod_base = OTHER; note = 2'-O-methylguanosine-3'-phosphate
  modified_base 12: mod_base = OTHER; note = 2'-O-methylguanosine-3'-phosphate
  modified_base 13: mod_base = OTHER; note = 2'-O-methyladenosine-3'-phosphate
  modified_base 14: mod_base = OTHER; note = 2'-deoxy-2'-fluorouridine-3'-phosphate
  modified_base 15: mod_base = OTHER; note = 2'-O-methyluridine-3'-phosphate
  modified_base 16: mod_base = OTHER; note = 2'-deoxy-2'-fluoroadenosine-3'-phosphate
  modified_base 17: mod_base = OTHER; note = 2'-O-methyladenosine-3'-phosphate
  modified_base 18: mod_base = OTHER; note = 2'-O-methyladenosine-3'-phosphate
  modified_base 19: mod_base = OTHER; note = 2'-O-methyladenosine-3'-phosphate
  modified_base 20: mod_base = OTHER; note = 2'-O-methyluridine-3'-phosphate
  modified_base 21: mod_base = OTHER; note = 2'-O-methylguanosine-3'-phosphorothioate
  modified_base 22: mod_base = OTHER; note = 2'-O-methyladenosine-3'-phosphorothioate
  modified_base 23: mod_base = OTHER; note = 2'-O-methylguanosine
tttagagtgaggattaaaatgag

SEQ ID NO: 26 - RNA, 21 nt, synthetic construct
  source 1..21: mol_type = other RNA
  modified_base 1: mod_base = OTHER; note = 2'-O-methylcytidine-3'-phosphorothioate
  modified_base 2: mod_base = OTHER; note = 2'-O-methyladenosine-3'-phosphorothioate
  modified_base 3: mod_base = OTHER; note = 2'-O-methyluridine-3'-phosphate
  modified_base 4: mod_base = OTHER; note = 2'-O-methyluridine-3'-phosphate
  modified_base 5: mod_base = OTHER; note = 2'-O-methyluridine-3'-phosphate
  modified_base 6: mod_base = OTHER; note = 2'-O-(2-oxo-2-(N-hexadec-1-ylamino)ethyl)uridine-3' -phosphate
  modified_base 7: mod_base = OTHER; note = 2'-deoxy-2'-fluoroadenosine-3'-phosphate
  modified_base 8: mod_base = OTHER; note = 2'-O-methyladenosine-3'-phosphate
  modified_base 9: mod_base = OTHER; note = 2'-deoxy-2'-fluorouridine-3'-phosphate
  modified_base 10: mod_base = OTHER; note = 2'-deoxy-2'-fluorocytidine-3'-phosphate
  modified_base 11: mod_base = OTHER; note = 2'-deoxy-2'-fluorocytidine-3'-phosphate
  modified_base 12: mod_base = OTHER; note = 2'-O-methyluridine-3'-phosphate
  modified_base 13: mod_base = OTHER; note = 2'-O-methylcytidine-3'-phosphate
  modified_base 14: mod_base = OTHER; note = 2'-O-methyladenosine-3'-phosphate
  modified_base 15: mod_base = OTHER; note = 2'-O-methylcytidine-3'-phosphate
  modified_base 16: mod_base = OTHER; note = 2'-O-methyluridine-3'-phosphate
  modified_base 17: mod_base = OTHER; note = 2'-O-methylcytidine-3'-phosphate
  modified_base 18: mod_base = OTHER; note = 2'-O-methyluridine-3'-phosphate
  modified_base 19: mod_base = OTHER; note = 2'-O-methyladenosine-3'-phosphorothioate
  modified_base 20: mod_base = OTHER; note = 2'-O-methyladenosine-3'-phosphorothioate
  modified_base 21: mod_base = OTHER; note = 2'-O-methyladenosine
cattttaatcctcactctaaa

SEQ ID NO: 27 - RNA, 23 nt, synthetic construct
  source 1..23: mol_type = other RNA
  modified_base 1: mod_base = OTHER; note = 2'-O-methyluridine-3'-phosphorothioate 5'-(E)-Vinylphosphonate
  modified_base 2: mod_base = OTHER; note = 2'-deoxy-2'-fluorouridine-3'-phosphorothioate
  modified_base 3: mod_base = OTHER; note = 2'-O-methyluridine-3'-phosphate
  modified_base 4: mod_base = OTHER; note = 2'-O-methyladenosine-3'-phosphate
  modified_base 5: mod_base = OTHER; note = 2'-O-methylguanosine-3'-phosphate
  modified_base 6: mod_base = OTHER; note = 2'-deoxy-2'-fluoroadenosine-3'-phosphate
  modified_base 7: mod_base = OTHER; note = 2'-O-methylguanosine-3'-phosphate
  modified_base 8: mod_base = OTHER; note = 2'-deoxy-2'-fluorouridine-3'-phosphate
  modified_base 9: mod_base = OTHER; note = 2'-deoxy-2'-fluoroguanosine-3'-phosphate
  modified_base 10: mod_base = OTHER; note = 2'-O-methyladenosine-3'-phosphate
  modified_base 11: mod_base = OTHER; note = 2'-O-methylguanosine-3'-phosphate
  modified_base 12: mod_base = OTHER; note = 2'-O-methylguanosine-3'-phosphate
  modified_base 13: mod_base = OTHER; note = 2'-O-methyladenosine-3'-phosphate
  modified_base 14: mod_base = OTHER; note = 2'-deoxy-2'-fluorouridine-3'-phosphate
  modified_base 15: mod_base = OTHER; note = 2'-O-methyluridine-3'-phosphate
  modified_base 16: mod_base = OTHER; note = 2'-deoxy-2'-fluoroadenosine-3'-phosphate
  modified_base 17: mod_base = OTHER; note = 2'-O-methyladenosine-3'-phosphate
  modified_base 18: mod_base = OTHER; note = 2'-O-methyladenosine-3'-phosphate
  modified_base 19: mod_base = OTHER; note = 2'-O-methyladenosine-3'-phosphate
  modified_base 20: mod_base = OTHER; note = 2'-O-methyluridine-3'-phosphate
  modified_base 21: mod_base = OTHER; note = 2'-O-methylguanosine-3'-phosphorothioate
  modified_base 22: mod_base = OTHER; note = 2'-O-methyladenosine-3'-phosphorothioate
  modified_base 23: mod_base = OTHER; note = 2'-O-methylguanosine
tttagagtgaggattaaaatgag

SEQ ID NO: 28 - RNA, 21 nt, synthetic construct
  source 1..21: mol_type = other RNA
  modified_base 1: mod_base = OTHER; note = 2'-O-methylcytidine-3'-phosphorothioate
  modified_base 2: mod_base = OTHER; note = 2'-O-methyladenosine-3'-phosphorothioate
  modified_base 3: mod_base = OTHER; note = 2'-O-methyluridine-3'-phosphate
  modified_base 4: mod_base = OTHER; note = 2'-O-methyluridine-3'-phosphate
  modified_base 5: mod_base = OTHER; note = 2'-O-methyluridine-3'-phosphate
  modified_base 6: mod_base = OTHER; note = 2'-O-(2-oxo-2-(N-docosan-1-ylamino)ethyl)uridine-3' -phosphate
  modified_base 7: mod_base = OTHER; note = 2'-deoxy-2'-fluoroadenosine-3'-phosphate
  modified_base 8: mod_base = OTHER; note = 2'-O-methyladenosine-3'-phosphate
  modified_base 9: mod_base = OTHER; note = 2'-deoxy-2'-fluorouridine-3'-phosphate
  modified_base 10: mod_base = OTHER; note = 2'-deoxy-2'-fluorocytidine-3'-phosphate
  modified_base 11: mod_base = OTHER; note = 2'-deoxy-2'-fluorocytidine-3'-phosphate
  modified_base 12: mod_base = OTHER; note = 2'-O-methyluridine-3'-phosphate
  modified_base 13: mod_base = OTHER; note = 2'-O-methylcytidine-3'-phosphate
  modified_base 14: mod_base = OTHER; note = 2'-O-methyladenosine-3'-phosphate
  modified_base 15: mod_base = OTHER; note = 2'-O-methylcytidine-3'-phosphate
  modified_base 16: mod_base = OTHER; note = 2'-O-methyluridine-3'-phosphate
  modified_base 17: mod_base = OTHER; note = 2'-O-methylcytidine-3'-phosphate
  modified_base 18: mod_base = OTHER; note = 2'-O-methyluridine-3'-phosphate
  modified_base 19: mod_base = OTHER; note = 2'-O-methyladenosine-3'-phosphorothioate
  modified_base 20: mod_base = OTHER; note = 2'-O-methyladenosine-3'-phosphorothioate
  modified_base 21: mod_base = OTHER; note = 2'-O-methyladenosine
cattttaatcctcactctaaa

SEQ ID NO: 29 - RNA, 23 nt, synthetic construct
  source 1..23: mol_type = other RNA
  modified_base 1: mod_base = OTHER; note = 2'-O-methyluridine-3'-phosphorothioate 5'-(E)-Vinylphosphonate
  modified_base 2: mod_base = OTHER; note = 2'-deoxy-2'-fluorouridine-3'-phosphorothioate
  modified_base 3: mod_base = OTHER; note = 2'-O-methyluridine-3'-phosphate
  modified_base 4: mod_base = OTHER; note = 2'-O-methyladenosine-3'-phosphate
  modified_base 5: mod_base = OTHER; note = 2'-O-methylguanosine-3'-phosphate
  modified_base 6: mod_base = OTHER; note = 2'-deoxy-2'-fluoroadenosine-3'-phosphate
  modified_base 7: mod_base = OTHER; note = 2'-O-methylguanosine-3'-phosphate
  modified_base 8: mod_base = OTHER; note = 2'-deoxy-2'-fluorouridine-3'-phosphate
  modified_base 9: mod_base = OTHER; note = 2'-deoxy-2'-fluoroguanosine-3'-phosphate
  modified_base 10: mod_base = OTHER; note = 2'-O-methyladenosine-3'-phosphate
  modified_base 11: mod_base = OTHER; note = 2'-O-methylguanosine-3'-phosphate
  modified_base 12: mod_base = OTHER; note = 2'-O-methylguanosine-3'-phosphate
  modified_base 13: mod_base = OTHER; note = 2'-O-methyladenosine-3'-phosphate
  modified_base 14: mod_base = OTHER; note = 2'-deoxy-2'-fluorouridine-3'-phosphate
  modified_base 15: mod_base = OTHER; note = 2'-O-methyluridine-3'-phosphate
  modified_base 16: mod_base = OTHER; note = 2'-deoxy-2'-fluoroadenosine-3'-phosphate
  modified_base 17: mod_base = OTHER; note = 2'-O-methyladenosine-3'-phosphate
  modified_base 18: mod_base = OTHER; note = 2'-O-methyladenosine-3'-phosphate
  modified_base 19: mod_base = OTHER; note = 2'-O-methyladenosine-3'-phosphate
  modified_base 20: mod_base = OTHER; note = 2'-O-methyluridine-3'-phosphate
  modified_base 21: mod_base = OTHER; note = 2'-O-methylguanosine-3'-phosphorothioate
  modified_base 22: mod_base = OTHER; note = 2'-O-methyladenosine-3'-phosphorothioate
  modified_base 23: mod_base = OTHER; note = 2'-O-methylguanosine
tttagagtgaggattaaaatgag

SEQ ID NO: 30-31 - skipped (2 entries)

SEQ ID NO: 32 - RNA, 21 nt, synthetic construct
  source 1..21: mol_type = other RNA
aacagtgttcttgctctataa

SEQ ID NO: 33 - RNA, 23 nt, synthetic construct
  source 1..23: mol_type = other RNA
ttatagagcaagaacactgtttt

SEQ ID NO: 34 - RNA, 21 nt, synthetic construct
  source 1..21: mol_type = other RNA
  modified_base 1: mod_base = OTHER; note = 2'-O-methyladenosine-3'-phosphorothioate
  modified_base 2: mod_base = OTHER; note = 2'-O-methyladenosine-3'-phosphorothioate
  modified_base 3: mod_base = OTHER; note = 2'-O-methylcytidine-3'-phosphate
  modified_base 4: mod_base = OTHER; note = 2'-O-methyladenosine-3'-phosphate
  modified_base 5: mod_base = OTHER; note = 2'-O-methylguanosine-3'-phosphate
  modified_base 6: mod_base = OTHER; note = 2'-O-hexadecyl-uridine-3'-phosphate
  modified_base 7: mod_base = OTHER; note = 2'-deoxy-2'-fluoroguanosine-3'-phosphate
  modified_base 8: mod_base = OTHER; note = 2'-O-methyluridine-3'-phosphate
  modified_base 9: mod_base = OTHER; note = 2'-deoxy-2'-fluorouridine-3'-phosphate
  modified_base 10: mod_base = OTHER; note = 2'-deoxy-2'-fluorocytidine-3'-phosphate
  modified_base 11: mod_base = OTHER; note = 2'-deoxy-2'-fluorouridine-3'-phosphate
  modified_base 12: mod_base = OTHER; note = 2'-O-methyluridine-3'-phosphate
  modified_base 13: mod_base = OTHER; note = 2'-O-methylguanosine-3'-phosphate
  modified_base 14: mod_base = OTHER; note = 2'-O-methylcytidine-3'-phosphate
  modified_base 15: mod_base = OTHER; note = 2'-O-methyluridine-3'-phosphate
  modified_base 16: mod_base = OTHER; note = 2'-O-methylcytidine-3'-phosphate
  modified_base 17: mod_base = OTHER; note = 2'-O-methyluridine-3'-phosphate
  modified_base 18: mod_base = OTHER; note = 2'-O-methyladenosine-3'-phosphate
  modified_base 19: mod_base = OTHER; note = 2'-O-methyluridine-3'-phosphorothioate
  modified_base 20: mod_base = OTHER; note = 2'-O-methyladenosine-3'-phosphorothioate
  modified_base 21: mod_base = OTHER; note = 2'-O-methyladenosine
aacagtgttcttgctctataa

SEQ ID NO: 35 - RNA, 23 nt, synthetic construct
  source 1..23: mol_type = other RNA
  modified_base 1: mod_base = OTHER; note = 2'-O-methyluridine-3'-phosphorothioate 5'-(E)-Vinylphosphonate
  modified_base 2: mod_base = OTHER; note = 2'-deoxy-2'-fluorouridine-3'-phosphorothioate
  modified_base 3: mod_base = OTHER; note = 2'-O-methyladenosine-3'-phosphate
  modified_base 4: mod_base = OTHER; note = 2'-O-methyluridine-3'-phosphate
  modified_base 5: mod_base = OTHER; note = 2'-O-methyladenosine-3'-phosphate
  modified_base 6: mod_base = OTHER; note = 2'-deoxy-2'-fluoroguanosine-3'-phosphate
  modified_base 7: mod_base = OTHER; note = 2'-O-methyladenosine-3'-phosphate
  modified_base 8: mod_base = OTHER; note = 2'-O-methylguanosine-3'-phosphate
  modified_base 9: mod_base = OTHER; note = 2'-O-methylcytidine-3'-phosphate
  modified_base 10: mod_base = OTHER; note = 2'-O-methyladenosine-3'-phosphate
  modified_base 11: mod_base = OTHER; note = 2'-O-methyladenosine-3'-phosphate
  modified_base 12: mod_base = OTHER; note = 2'-O-methylguanosine-3'-phosphate
  modified_base 13: mod_base = OTHER; note = 2'-O-methyladenosine-3'-phosphate
  modified_base 14: mod_base = OTHER; note = 2'-deoxy-2'-fluoroadenosine-3'-phosphate
  modified_base 15: mod_base = OTHER; note = 2'-O-methylcytidine-3'-phosphate
  modified_base 16: mod_base = OTHER; note = 2'-deoxy-2'-fluoroadenosine-3'-phosphate
  modified_base 17: mod_base = OTHER; note = 2'-O-methylcytidine-3'-phosphate
  modified_base 18: mod_base = OTHER; note = 2'-O-methyluridine-3'-phosphate
  modified_base 19: mod_base = OTHER; note = 2'-O-methylguanosine-3'-phosphate
  modified_base 20: mod_base = OTHER; note = 2'-O-methyluridine-3'-phosphate
  modified_base 21: mod_base = OTHER; note = 2'-O-methyluridine-3'-phosphorothioate
  modified_base 22: mod_base = OTHER; note = 2'-O-methyluridine-3'-phosphorothioate
  modified_base 23: mod_base = OTHER; note = 2'-O-methyluridine
ttatagagcaagaacactgtttt

SEQ ID NO: 36 - RNA, 21 nt, synthetic construct
  source 1..21: mol_type = other RNA
  modified_base 1: mod_base = OTHER; note = 2'-O-methyladenosine-3'-phosphorothioate
  modified_base 2: mod_base = OTHER; note = 2'-O-methyladenosine-3'-phosphorothioate
  modified_base 3: mod_base = OTHER; note = 2'-O-methylcytidine-3'-phosphate
  modified_base 4: mod_base = OTHER; note = 2'-O-methyladenosine-3'-phosphate
  modified_base 5: mod_base = OTHER; note = 2'-O-methylguanosine-3'-phosphate
  modified_base 6: mod_base = OTHER; note = 2'-O-(2-(1-hexadecyloxy)ethyl)uridine-3'-phosphate
  modified_base 7: mod_base = OTHER; note = 2'-deoxy-2'-fluoroguanosine-3'-phosphate
  modified_base 8: mod_base = OTHER; note = 2'-O-methyluridine-3'-phosphate
  modified_base 9: mod_base = OTHER; note = 2'-deoxy-2'-fluorouridine-3'-phosphate
  modified_base 10: mod_base = OTHER; note = 2'-deoxy-2'-fluorocytidine-3'-phosphate
  modified_base 11: mod_base = OTHER; note = 2'-deoxy-2'-fluorouridine-3'-phosphate
  modified_base 12: mod_base = OTHER; note = 2'-O-methyluridine-3'-phosphate
  modified_base 13: mod_base = OTHER; note = 2'-O-methylguanosine-3'-phosphate
  modified_base 14: mod_base = OTHER; note = 2'-O-methylcytidine-3'-phosphate
  modified_base 15: mod_base = OTHER; note = 2'-O-methyluridine-3'-phosphate
  modified_base 16: mod_base = OTHER; note = 2'-O-methylcytidine-3'-phosphate
  modified_base 17: mod_base = OTHER; note = 2'-O-methyluridine-3'-phosphate
  modified_base 18: mod_base = OTHER; note = 2'-O-methyladenosine-3'-phosphate
  modified_base 19: mod_base = OTHER; note = 2'-O-methyluridine-3'-phosphorothioate
  modified_base 20: mod_base = OTHER; note = 2'-O-methyladenosine-3'-phosphorothioate
  modified_base 21: mod_base = OTHER; note = 2'-O-methyladenosine
aacagtgttcttgctctataa

SEQ ID NO: 37 - RNA, 23 nt, synthetic construct
  source 1..23: mol_type = other RNA
  modified_base 1: mod_base = OTHER; note = 2'-O-methyluridine-3'-phosphorothioate 5'-(E)-Vinylphosphonate
  modified_base 2: mod_base = OTHER; note = 2'-deoxy-2'-fluorouridine-3'-phosphorothioate
  modified_base 3: mod_base = OTHER; note = 2'-O-methyladenosine-3'-phosphate
  modified_base 4: mod_base = OTHER; note = 2'-O-methyluridine-3'-phosphate
  modified_base 5: mod_base = OTHER; note = 2'-O-methyladenosine-3'-phosphate
  modified_base 6: mod_base = OTHER; note = 2'-deoxy-2'-fluoroguanosine-3'-phosphate
  modified_base 7: mod_base = OTHER; note = 2'-O-methyladenosine-3'-phosphate
  modified_base 8: mod_base = OTHER; note = 2'-O-methylguanosine-3'-phosphate
  modified_base 9: mod_base = OTHER; note = 2'-O-methylcytidine-3'-phosphate
  modified_base 10: mod_base = OTHER; note = 2'-O-methyladenosine-3'-phosphate
  modified_base 11: mod_base = OTHER; note = 2'-O-methyladenosine-3'-phosphate
  modified_base 12: mod_base = OTHER; note = 2'-O-methylguanosine-3'-phosphate
  modified_base 13: mod_base = OTHER; note = 2'-O-methyladenosine-3'-phosphate
  modified_base 14: mod_base = OTHER; note = 2'-deoxy-2'-fluoroadenosine-3'-phosphate
  modified_base 15: mod_base = OTHER; note = 2'-O-methylcytidine-3'-phosphate
  modified_base 16: mod_base = OTHER; note = 2'-deoxy-2'-fluoroadenosine-3'-phosphate
  modified_base 17: mod_base = OTHER; note = 2'-O-methylcytidine-3'-phosphate
  modified_base 18: mod_base = OTHER; note = 2'-O-methyluridine-3'-phosphate
  modified_base 19: mod_base = OTHER; note = 2'-O-methylguanosine-3'-phosphate
  modified_base 20: mod_base = OTHER; note = 2'-O-methyluridine-3'-phosphate
  modified_base 21: mod_base = OTHER; note = 2'-O-methyluridine-3'-phosphorothioate
  modified_base 22: mod_base = OTHER; note = 2'-O-methyluridine-3'-phosphorothioate
  modified_base 23: mod_base = OTHER; note = 2'-O-methyluridine
ttatagagcaagaacactgtttt

SEQ ID NO: 38 - RNA, 21 nt, synthetic construct
  source 1..21: mol_type = other RNA
  modified_base 1: mod_base = OTHER; note = 2'-O-methyladenosine-3'-phosphorothioate
  modified_base 2: mod_base = OTHER; note = 2'-O-methyladenosine-3'-phosphorothioate
  modified_base 3: mod_base = OTHER; note = 2'-O-methylcytidine-3'-phosphate
  modified_base 4: mod_base = OTHER; note = 2'-O-methyladenosine-3'-phosphate
  modified_base 5: mod_base = OTHER; note = 2'-O-methylguanosine-3'-phosphate
  modified_base 6: mod_base = OTHER; note = 2'-O-(2-oxo-2-(N-hexadec-1-ylamino)ethyl)uridine-3' -phosphate
  modified_base 7: mod_base = OTHER; note = 2'-deoxy-2'-fluoroguanosine-3'-phosphate
  modified_base 8: mod_base = OTHER; note = 2'-O-methyluridine-3'-phosphate
  modified_base 9: mod_base = OTHER; note = 2'-deoxy-2'-fluorouridine-3'-phosphate
  modified_base 10: mod_base = OTHER; note = 2'-deoxy-2'-fluorocytidine-3'-phosphate
  modified_base 11: mod_base = OTHER; note = 2'-deoxy-2'-fluorouridine-3'-phosphate
  modified_base 12: mod_base = OTHER; note = 2'-O-methyluridine-3'-phosphate
  modified_base 13: mod_base = OTHER; note = 2'-O-methylguanosine-3'-phosphate
  modified_base 14: mod_base = OTHER; note = 2'-O-methylcytidine-3'-phosphate
  modified_base 15: mod_base = OTHER; note = 2'-O-methyluridine-3'-phosphate
  modified_base 16: mod_base = OTHER; note = 2'-O-methylcytidine-3'-phosphate
  modified_base 17: mod_base = OTHER; note = 2'-O-methyluridine-3'-phosphate
  modified_base 18: mod_base = OTHER; note = 2'-O-methyladenosine-3'-phosphate
  modified_base 19: mod_base = OTHER; note = 2'-O-methyluridine-3'-phosphorothioate
  modified_base 20: mod_base = OTHER; note = 2'-O-methyladenosine-3'-phosphorothioate
  modified_base 21: mod_base = OTHER; note = 2'-O-methyladenosine
aacagtgttcttgctctataa

SEQ ID NO: 39 - RNA, 23 nt, synthetic construct
  source 1..23: mol_type = other RNA
  modified_base 1: mod_base = OTHER; note = 2'-O-methyluridine-3'-phosphorothioate 5'-(E)-Vinylphosphonate
  modified_base 2: mod_base = OTHER; note = 2'-deoxy-2'-fluorouridine-3'-phosphorothioate
  modified_base 3: mod_base = OTHER; note = 2'-O-methyladenosine-3'-phosphate
  modified_base 4: mod_base = OTHER; note = 2'-O-methyluridine-3'-phosphate
  modified_base 5: mod_base = OTHER; note = 2'-O-methyladenosine-3'-phosphate
  modified_base 6: mod_base = OTHER; note = 2'-deoxy-2'-fluoroguanosine-3'-phosphate
  modified_base 7: mod_base = OTHER; note = 2'-O-methyladenosine-3'-phosphate
  modified_base 8: mod_base = OTHER; note = 2'-O-methylguanosine-3'-phosphate
  modified_base 9: mod_base = OTHER; note = 2'-O-methylcytidine-3'-phosphate
  modified_base 10: mod_base = OTHER; note = 2'-O-methyladenosine-3'-phosphate
  modified_base 11: mod_base = OTHER; note = 2'-O-methyladenosine-3'-phosphate
  modified_base 12: mod_base = OTHER; note = 2'-O-methylguanosine-3'-phosphate
  modified_base 13: mod_base = OTHER; note = 2'-O-methyladenosine-3'-phosphate
  modified_base 14: mod_base = OTHER; note = 2'-deoxy-2'-fluoroadenosine-3'-phosphate
  modified_base 15: mod_base = OTHER; note = 2'-O-methylcytidine-3'-phosphate
  modified_base 16: mod_base = OTHER; note = 2'-deoxy-2'-fluoroadenosine-3'-phosphate
  modified_base 17: mod_base = OTHER; note = 2'-O-methylcytidine-3'-phosphate
  modified_base 18: mod_base = OTHER; note = 2'-O-methyluridine-3'-phosphate
  modified_base 19: mod_base = OTHER; note = 2'-O-methylguanosine-3'-phosphate
  modified_base 20: mod_base = OTHER; note = 2'-O-methyluridine-3'-phosphate
  modified_base 21: mod_base = OTHER; note = 2'-O-methyluridine-3'-phosphorothioate
  modified_base 22: mod_base = OTHER; note = 2'-O-methyluridine-3'-phosphorothioate
  modified_base 23: mod_base = OTHER; note = 2'-O-methyluridine
ttatagagcaagaacactgtttt

SEQ ID NO: 40 - RNA, 21 nt, synthetic construct
  source 1..21: mol_type = other RNA
  modified_base 1: mod_base = OTHER; note = 2'-O-methyladenosine-3'-phosphorothioate
  modified_base 2: mod_base = OTHER; note = 2'-O-methyladenosine-3'-phosphorothioate
  modified_base 3: mod_base = OTHER; note = 2'-O-methylcytidine-3'-phosphate
  modified_base 4: mod_base = OTHER; note = 2'-O-methyladenosine-3'-phosphate
  modified_base 5: mod_base = OTHER; note = 2'-O-methylguanosine-3'-phosphate
  modified_base 6: mod_base = OTHER; note = 2'-O-(15-carboxypentadec-1-yl)uridine-3'-phosphate
  modified_base 7: mod_base = OTHER; note = 2'-deoxy-2'-fluoroguanosine-3'-phosphate
  modified_base 8: mod_base = OTHER; note = 2'-O-methyluridine-3'-phosphate
  modified_base 9: mod_base = OTHER; note = 2'-deoxy-2'-fluorouridine-3'-phosphate
  modified_base 10: mod_base = OTHER; note = 2'-deoxy-2'-fluorocytidine-3'-phosphate
  modified_base 11: mod_base = OTHER; note = 2'-deoxy-2'-fluorouridine-3'-phosphate
  modified_base 12: mod_base = OTHER; note = 2'-O-methyluridine-3'-phosphate
  modified_base 13: mod_base = OTHER; note = 2'-O-methylguanosine-3'-phosphate
  modified_base 14: mod_base = OTHER; note = 2'-O-methylcytidine-3'-phosphate
  modified_base 15: mod_base = OTHER; note = 2'-O-methyluridine-3'-phosphate
  modified_base 16: mod_base = OTHER; note = 2'-O-methylcytidine-3'-phosphate
  modified_base 17: mod_base = OTHER; note = 2'-O-methyluridine-3'-phosphate
  modified_base 18: mod_base = OTHER; note = 2'-O-methyladenosine-3'-phosphate
  modified_base 19: mod_base = OTHER; note = 2'-O-methyluridine-3'-phosphorothioate
  modified_base 20: mod_base = OTHER; note = 2'-O-methyladenosine-3'-phosphorothioate
  modified_base 21: mod_base = OTHER; note = 2'-O-methyladenosine
aacagtgttcttgctctataa

SEQ ID NO: 41 - RNA, 23 nt, synthetic construct
  source 1..23: mol_type = other RNA
  modified_base 1: mod_base = OTHER; note = 2'-O-methyluridine-3'-phosphorothioate 5'-(E)-Vinylphosphonate
  modified_base 2: mod_base = OTHER; note = 2'-deoxy-2'-fluorouridine-3'-phosphorothioate
  modified_base 3: mod_base = OTHER; note = 2'-O-methyladenosine-3'-phosphate
  modified_base 4: mod_base = OTHER; note = 2'-O-methyluridine-3'-phosphate
  modified_base 5: mod_base = OTHER; note = 2'-O-methyladenosine-3'-phosphate
  modified_base 6: mod_base = OTHER; note = 2'-deoxy-2'-fluoroguanosine-3'-phosphate
  modified_base 7: mod_base = OTHER; note = 2'-O-methyladenosine-3'-phosphate
  modified_base 8: mod_base = OTHER; note = 2'-O-methylguanosine-3'-phosphate
  modified_base 9: mod_base = OTHER; note = 2'-O-methylcytidine-3'-phosphate
  modified_base 10: mod_base = OTHER; note = 2'-O-methyladenosine-3'-phosphate
  modified_base 11: mod_base = OTHER; note = 2'-O-methyladenosine-3'-phosphate
  modified_base 12: mod_base = OTHER; note = 2'-O-methylguanosine-3'-phosphate
  modified_base 13: mod_base = OTHER; note = 2'-O-methyladenosine-3'-phosphate
  modified_base 14: mod_base = OTHER; note = 2'-deoxy-2'-fluoroadenosine-3'-phosphate
  modified_base 15: mod_base = OTHER; note = 2'-O-methylcytidine-3'-phosphate
  modified_base 16: mod_base = OTHER; note = 2'-deoxy-2'-fluoroadenosine-3'-phosphate
  modified_base 17: mod_base = OTHER; note = 2'-O-methylcytidine-3'-phosphate
  modified_base 18: mod_base = OTHER; note = 2'-O-methyluridine-3'-phosphate
  modified_base 19: mod_base = OTHER; note = 2'-O-methylguanosine-3'-phosphate
  modified_base 20: mod_base = OTHER; note = 2'-O-methyluridine-3'-phosphate
  modified_base 21: mod_base = OTHER; note = 2'-O-methyluridine-3'-phosphorothioate
  modified_base 22: mod_base = OTHER; note = 2'-O-methyluridine-3'-phosphorothioate
  modified_base 23: mod_base = OTHER; note = 2'-O-methyluridine
ttatagagcaagaacactgtttt

SEQ ID NO: 42 - protein, 29 aa, unidentified
AALEALAEALEALAEALEALAEAAAAGGC

SEQ ID NO: 43 - protein, 30 aa, unidentified
AALAEALAEALAEALAEALAEALAAAAGGC

SEQ ID NO: 44 - protein, 15 aa, unidentified
ALEALAEALEALAEA

SEQ ID NO: 45 - protein, 22 aa, unidentified
GLFEAIEGFIENGWEGMIWDYG

SEQ ID NO: 46 - protein, 23 aa, unidentified
GLFGAIAGFIENGWEGMIDGWYG

SEQ ID NO: 47 - protein, 43 aa, unidentified
GLFEAIEGFIENGWEGMIDGWYGCGLFEAIEGFIENGWEGMID

SEQ ID NO: 48 - protein, 44 aa, unidentified
GLFEAIEGFIENGWEGMIDGGCGLFEAIEGFIENGWEGMIDGGC

SEQ ID NO: 49 - protein, 35 aa, unidentified
GLFGALAEALAEALAEHLAEALAEALEALAAGGSC

SEQ ID NO: 50 - protein, 34 aa, unidentified
GLFEAIEGFIENGWEGLAEALAEALEALAAGGSC

SEQ ID NO: 51 - protein, 41 aa, unidentified
  SITE 17: note = Norleucine
  SITE 38: note = Norleucine
GLFEAIEGFIENGWEGXIDGKGLFEAIEGFIENGWEGXIDG

SEQ ID NO: 52 - protein, 19 aa, unidentified
LFEALLELLESLWELLLEA

SEQ ID NO: 53 - protein, 20 aa, unidentified
GLFKALLKLLKSLWKLLLKA

SEQ ID NO: 54 - protein, 20 aa, unidentified
GLFRALLRLLRSLWRLLLRA

SEQ ID NO: 55 - protein, 30 aa, unidentified
WEAKLAKALAKALAKHLAKALAKALKACEA

SEQ ID NO: 56 - protein, 22 aa, unidentified
GLFFEAIAEFIEGGWEGLIEGC

SEQ ID NO: 57 - protein, 26 aa, unidentified
GIGAVLKVLTTGLPALISWIKRKRQQ

SEQ ID NO: 58 - protein, 8 aa, unidentified
HHHHHWYG

SEQ ID NO: 59 - protein, 10 aa, unidentified
CHKKKKKKHC

SEQ ID NO: 60 - RNA, 21 nt, synthetic construct
  source 1..21: mol_type = other RNA
  modified_base 1: mod_base = OTHER; note = 2'-O-methylcytidine-3'-phosphorothioate
  modified_base 2: mod_base = OTHER; note = 2'-O-methyladenosine-3'-phosphorothioate
  modified_base 3: mod_base = OTHER; note = 2'-O-methyluridine-3'-phosphate
  modified_base 4: mod_base = OTHER; note = 2'-O-methyluridine-3'-phosphate
  modified_base 5: mod_base = OTHER; note = 2'-O-methyluridine-3'-phosphate
  modified_base 6: mod_base = OTHER; note = 2'-O-hexadecyl-uridine-3'-phosphate
  modified_base 7: mod_base = OTHER; note = 2'-deoxy-2'-fluoroadenosine-3'-phosphate
  modified_base 8: mod_base = OTHER; note = 2'-O-methyladenosine-3'-phosphate
  modified_base 9: mod_base = OTHER; note = 2'-deoxy-2'-fluorouridine-3'-phosphate
  modified_base 10: mod_base = OTHER; note = 2'-deoxy-2'-fluorocytidine-3'-phosphate
  modified_base 11: mod_base = OTHER; note = 2'-deoxy-2'-fluorocytidine-3'-phosphate
  modified_base 12: mod_base = OTHER; note = 2'-O-methyluridine-3'-phosphate
  modified_base 13: mod_base = OTHER; note = 2'-O-methylcytidine-3'-phosphate
  modified_base 14: mod_base = OTHER; note = 2'-O-methyladenosine-3'-phosphate
  modified_base 15: mod_base = OTHER; note = 2'-O-methylcytidine-3'-phosphate
  modified_base 16: mod_base = OTHER; note = 2'-O-methyluridine-3'-phosphate
  modified_base 17: mod_base = OTHER; note = 2'-O-methylcytidine-3'-phosphate
  modified_base 18: mod_base = OTHER; note = 2'-O-methyluridine-3'-phosphate
  modified_base 19: mod_base = OTHER; note = 2'-O-methyladenosine-3'-phosphorothioate
  modified_base 20: mod_base = OTHER; note = 2'-O-methyladenosine-3'-phosphorothioate
  modified_base 21: mod_base = OTHER; note = 2'-O-methyladenosine
cattttaatcctcactctaaa

SEQ ID NO: 61 - RNA, 23 nt, synthetic construct
  source 1..23: mol_type = other RNA
  modified_base 1: mod_base = OTHER; note = 2'-O-methyluridine-3'-phosphorothioate 5'-(E)-Vinylphosphonate
  modified_base 2: mod_base = OTHER; note = 2'-deoxy-2'-fluorouridine-3'-phosphorothioate
  modified_base 3: mod_base = OTHER; note = 2'-O-methyluridine-3'-phosphate
  modified_base 4: mod_base = OTHER; note = 2'-O-methyladenosine-3'-phosphate
  modified_base 5: mod_base = OTHER; note = 2'-O-methylguanosine-3'-phosphate
  modified_base 6: mod_base = OTHER; note = 2'-deoxy-2'-fluoroadenosine-3'-phosphate
  modified_base 7: mod_base = OTHER; note = 2'-O-methylguanosine-3'-phosphate
  modified_base 8: mod_base = OTHER; note = 2'-deoxy-2'-fluorouridine-3'-phosphate
  modified_base 9: mod_base = OTHER; note = 2'-deoxy-2'-fluoroguanosine-3'-phosphate
  modified_base 10: mod_base = OTHER; note = 2'-O-methyladenosine-3'-phosphate
  modified_base 11: mod_base = OTHER; note = 2'-O-methylguanosine-3'-phosphate
  modified_base 12: mod_base = OTHER; note = 2'-O-methylguanosine-3'-phosphate
  modified_base 13: mod_base = OTHER; note = 2'-O-methyladenosine-3'-phosphate
  modified_base 14: mod_base = OTHER; note = 2'-deoxy-2'-fluorouridine-3'-phosphate
  modified_base 15: mod_base = OTHER; note = 2'-O-methyluridine-3'-phosphate
  modified_base 16: mod_base = OTHER; note = 2'-deoxy-2'-fluoroadenosine-3'-phosphate
  modified_base 17: mod_base = OTHER; note = 2'-O-methyladenosine-3'-phosphate
  modified_base 18: mod_base = OTHER; note = 2'-O-methyladenosine-3'-phosphate
  modified_base 19: mod_base = OTHER; note = 2'-O-methyladenosine-3'-phosphate
  modified_base 20: mod_base = OTHER; note = 2'-O-methyluridine-3'-phosphate
  modified_base 21: mod_base = OTHER; note = 2'-O-methylguanosine-3'-phosphorothioate
  modified_base 22: mod_base = OTHER; note = 2'-O-methyladenosine-3'-phosphorothioate
  modified_base 23: mod_base = OTHER; note = 2'-O-methylguanosine
tttagagtgaggattaaaatgag

(19) United States
(12) Patent Application Publication
(10) Pub. No.: US 2025/0382324 A1
(43) Pub. Date: Dec. 18, 2025

(54) MONOMERS AND METHODS FOR SYNTHESIS OF MODIFIED OLIGONUCLEOTIDES

(71) Applicant: ALNYLAM PHARMACEUTICALS, INC., Cambridge, MA (US)

(72) Inventors: Muthiah MANOHARAN, Cambridge, MA (US); Dhrubajyoti DATTA, Cambridge, MA (US); Masaaki NAKATA, Cambridge, MA (US); Christopher THEILE, Cambridge, MA (US)

(73) Assignee: ALNYLAM PHARMACEUTICALS, INC., Cambridge, MA (US)

(21) Appl. No.: 18/878,308

(22) PCT Filed: Jun. 29, 2023
(86) PCT No.: PCT/US2023/069445

§ 371 Date: Dec. 23, 2024

Related U.S. Application Data
(60) Provisional application No. 63/357,379, filed on Jun. 30, 2022.

(51) Int. Cl. C07H 21/02; C07H 1/00; C07H 19/067; C07H 19/10; C07H 19/167; C07H 19/20; C07H 21/04; C12N 15/113

(52) U.S. Cl. CPC C07H 21/02; C07H 1/00; C07H 19/067; C07H 19/10; C07H 19/167; C07H 19/20; C07H 21/04; C12N 15/113; C12N 2310/14

(57) ABSTRACT

The present disclosure relates to monomers and methods for synthesizing modified oligonucleotides.

### CROSS-REFERENCE TO RELATED APPLICATIONS

This application is a 35 U.S.C. § 371 National Phase Entry Application of International Application No. PCT/US2023/069445 filed Jun. 29, 2023, which designates the U.S. and claims benefit under 35 U.S.C. § 119(e) of U.S. Provisional Application No. 63/357,379, filed Jun. 30, 2022, the contents of all of which are incorporated herein by reference in their entireties.

### SEQUENCE LISTING

The instant application contains a Sequence Listing that has been submitted in XML format via Patent Center and is hereby incorporated by reference in its entirety. Said XML copy, created on Aug. 14, 2023, is named “ALN-459-WO.xml” and is 440,831 bytes in size.

### TECHNICAL FIELD

The present disclosure relates generally to monomers and methods for synthesis of modified oligonucleotides, e.g., single-stranded oligonucleotides and dsRNAs comprising such monomers.

### BACKGROUND

RNA interference or “RNAi” is a term initially coined by Fire and co-workers to describe the observation that double-stranded RNAi (dsRNA) can block gene expression (Fire et al. (1998) Nature 391, 806-811; Elbashir et al. (2001) Genes Dev. 15, 188-200). Short dsRNA directs gene-specific, post-transcriptional silencing in many organisms, including vertebrates, and has provided a new tool for studying gene function. RNAi is mediated by RNA-induced silencing complex (RISC), a sequence-specific, multi-component nuclease that destroys messenger RNAs homologous to the silencing trigger. RISC is known to contain short RNAs (approximately 22 nucleotides) derived from the double-stranded RNA trigger, but the protein components of this activity remained unknown.

There remains a need in the art for effective nucleotide or chemical motifs for dsRNA molecules, which are advantageous for inhibition of target gene expression. This invention is directed to that effort.

### SUMMARY

In one aspect, provided herein is a compound of Formula (I):

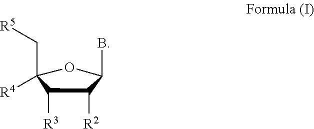

In compounds of Formula (I), at least one of R², R³, R⁴ and R⁵ is R^{MA}. Optionally, only one of R², R³, R⁴ and R⁵ is R^{MA}. Accordingly, in some embodiments of the various aspects described herein, one and only one of R², R³, R⁴ and R⁵ is R^{MA}.

In the various aspects described herein R^{MA} can be —O(CH₂)ₘ₁—X^{M′}—R^{M′} or —O(CH₂)ₙ₁—C(Y^{M})N(R^{N′})(R^{N″}), where Y^{M} is O or S; m1 is an integer from 1 to 10; and n1 is an integer from 1 to 10. In some embodiments, R^{MA} is —O(CH₂)ₘ₁—X^{M′}—R^{M′}. In some other embodiments, R^{MA} is or —O(CH₂)ₙ₁'C(Y^{M})N(R^{N′})(R^{N″}).

In the various aspects described herein, X^{M′} is N(R^{MX}), O or S, wherein R^{MX} is hydrogen or R^{M′}. Accordingly, in some embodiments of the any one of the aspects described herein X^{M′} is O. In some other embodiments of the any one of the aspects described herein X^{M′} is S. In yet some other embodiments, X^{M′} is N(R^{MX}).

In the various aspects described herein, R^{M′} can be optionally substituted C₆₋₃₀ alkyl, optionally substituted C₆₋₃₀alkenyl, optionally substituted C₆₋₃₀alkynyl, optionally substituted 3-8 membered heterocyclylC₃₋₃₀alkyl, optionally substituted C₃₋₁₀cycloalkylC₃₋₃₀alkyl; optionally substituted arylC₃₋₃₀alkyl, optionally substituted heteroarylC₃₋₃₀alkyl, optionally substituted C₁₋₃₀ alkoxyC₁₋₃₀alkyl, —(CH₂CH₂O)_{mq}—R^{MQ}, a lipid, a ligand (e.g., a targeting ligand (e.g., GalNac) or a pharmacokinetics modifier), a linker, or a linker to one or more ligands, wherein mq is an integer selected from 1-10 and R^{MQ} is hydrogen or C₁₋₆alkyl. In some embodiments, mq is 1, 2, 3, 4, 5, 6, 7, 8, 9 or 10.

In some embodiments of any one of the aspects described herein, R^{M′} is a lipid, a ligand, a linker, or a linker to one or more ligands. For example, R^{M′} is a ligand or a linker to one or more ligands. In some embodiments, R^{M′} is a ligand or a linker to one or more ligands.

In some embodiments of the any one of the aspects described herein, R^{M′} is optionally substituted C₆₋₃₀ alkyl, optionally substituted C₆₋₃₀alkenyl, optionally substituted C₆₋₃₀alkynyl, or optionally substituted C₃₋₃₀cycloalkyl. For example, R^{M′} is optionally substituted C₆₋₃₀ alkyl or optionally substituted C₆₋₃₀alkenyl. In some embodiments, R^{M′} is an optionally substituted C₆₋₃₀ alkyl. For example, R^{M′} is an optionally substituted C₆₋₃₀ alkyl, where the alkyl is substituted with at least one substituent.

In some embodiments of any one of the aspects described herein, R^{M′} is terminally substituted with an anionic group or a cationic group. For example, R^{M′} is C₆₋₃₀ alkyl, C₆₋₃₀alkenyl, or C₆₋₃₀alkynyl, where the C₆₋₃₀ alkyl, C₆₋₃₀alkenyl and C₆₋₃₀alkynyl is substituted at a terminal position with an anionic group or cationic group, and each of C₆₋₃₀ alkyl, C₆₋₃₀alkenyl and C₆₋₃₀alkynyl can be further optionally substituted. Exemplary anionic groups include, but are not limited to, carboxylate, carbonate, thiocarbonate, dithiocarbonate, phosphate, phosphonate, sulfate, sulfonate, nitrate, and borate. Exemplary cationic groups include, but are not limited to amines, ammonium groups, guanidinium groups, histidines, polyamines, pyridinium groups, and sulfonium groups.

In the various aspects described herein, m1 is an integer from 1 to 10, e.g., m1 can be 1, 2, 3, 4, 5, 6, 7, 8, 9 or 10. For example, m1 is 2, 3, 4, 5, 6, 7 or 8. Accordingly, in some embodiments of the any one of the aspects described herein m1 is 2. In some other embodiments of the any one of the aspects described herein m1 is 8.

In the various aspects described herein, n1 is an integer from 1 to 10, e.g., n1 can be 1, 2, 3, 4, 5, 6, 7, 8, 9 or 10. For example, n1 is 1 or 2. Accordingly, in some embodiments of the any one of the aspects described herein n1 is 1. In some other embodiments of the any one of the aspects described herein n1 is 2.

In the various aspects described herein, R^{N′} and R^{N″} independently are independently are hydrogen, optionally substituted C₆₋₃₀ alkyl, optionally substituted C₆₋₃₀alkenyl, optionally substituted C₆₋₃₀alkynyl, or optionally substituted C₃₋₃₀cycloalkyl, a lipid, a ligand (e.g., a targeting ligand (e.g., GalNac) or a pharmacokinetics modifier), a linker, or a linker to one or more ligands, provided that at least one of R^{N′} and R^{N″} is not H. In some embodiments of any one of the aspects described herein, R^{N′} and R^{N″} independently are hydrogen, a lipid, a ligand, a linker, or a linker to one or more ligands, provided that at least one of R^{N′} and R^{N″} is not H. For example, R^{N′} and R^{N″} independently are hydrogen, a ligand or a linker to one or more ligands, provided that at least one of R^{N′} and R^{N″} is not H.

In some embodiments of any one of the aspects described herein, at least one of R^{N′} and R^{N″} is a lipid, a ligand, a linker, or a linker to one or more ligands. For example, at least one at least one of R^{N′} and R^{N″} is a ligand or a linker to one or more ligands.

In compounds of Formula (I), B is an optionally modified nucleobase. For example, B can be natural or non-natural nucleobase, each of which can be optionally modified with one or more of functional groups, ligands, protecting groups and the like. In some embodiments, B is an unmodified nucleobase. In some other embodiments, B is a modified nucleobase.

In compounds of Formula (I), R² is —O(CH₂)ₘ₁—X^{M′}—R^{M′}, —O(CH₂)ₙ₁—C(Y^{M})N(R^{N′})(R^{N″}), hydrogen, hydroxyl, protected hydroxyl, phosphate group, reactive phosphorous group, halogen, optionally substituted C₁₋₃₀ alkyl, optionally substituted C₂₋₃₀alkenyl, optionally substituted C₂₋₃₀alkynyl, optionally substituted C₁₋₃₀alkoxy (e.g., methoxy), alkoxyalkyl (e.g., 2-methoxyethyl), alkoxyalkylamine, alkoxyoxycarboxylate, amino, alkylamino, dialkylamino, 5-8 membered heterocyclyl, —O—C₄₋₃₀alkyl-ON(CH₂R⁸)(CH₂R⁹), —O—C₄₋₃₀alkyl-ON(CH₂R⁸)(CH₂R⁹), a ligand, a linker covalently bonded to one or more ligands, a solid support, a linker or a linker covalently bonded to a solid support.

In some embodiments of the any one of the aspects described herein, R² is —O(CH₂)ₘ₁—X^{M′}—R^{M′}. For example, R² is —O(CH₂)ₘ₁—O—R^{M′}. In another non-limiting example, R² is —O(CH₂)ₘ₁—S—R^{M′}.

In some embodiments of the any one of the aspects described herein, R² is —OCH₂CH₂—X^{M′}—R^{M′}. For example, R² is —OCH₂CH₂—O—R^{M′} In another non-limiting example, R² is —OCH₂CH₂—S—R^{M′}.

In some embodiments of the any one of the aspects described herein, R² is —O(CH₂)ₙ₁—C(Y^{M})N(R^{N′})(R^{N″}). For example, R² is —O(CH₂)ₙ₁—C(O)N(R^{N′})(R^{N″}). In some embodiments, R² is —OCH₂—C(O)N(R^{N′})(R^{N″}). In some other embodiments, R² is —OCH₂CH₂—C(O)N(R^{N′})(R^{N″}).

In some embodiments of the any one of the aspects described herein, R² is hydrogen, hydroxyl, protected hydroxyl, halogen, optionally substituted C₁₋₃₀ alkoxy, a reactive phosphorous group, a solid support, a linker, or a linker covalently attached to a solid support. For example, wherein R² is hydrogen, hydroxyl, protected hydroxyl, halogen, a reactive phosphorous group, a solid support, a linker, or a linker covalently attached to a solid support. In some embodiments of the any one of the aspects described herein, R² is hydrogen, hydroxyl, protected hydroxyl, a reactive phosphorous group, a solid support, a linker, or a linker covalently attached to a solid support. For example, R² is a reactive phosphorous group, a solid support, a linker, or a linker covalently attached to a solid support. In some embodiments of the any one of the aspects described herein, R² is a reactive phosphorous or a linker covalently attached to a solid support. For example, R² is a reactive phosphorous group (e.g., a phosphoramidite, such as [(2-cyanoethyl)-(N,N-diisopropyl)]-phosphoramidite or [(ß-thiobenzoylethyl)-(1-pyrrolidinyl)]-thiophosphoramidite.

In compounds of Formula (I), R³ is —O(CH₂)ₘ₁—X^{M′}—R^{M′}, —O(CH₂)ₙ₁—C(Y^{M})N(R^{N′})(R^{N″}), hydrogen, hydroxyl, protected hydroxyl, phosphate group, reactive phosphorous group, halogen, optionally substituted C₁₋₃₀ alkyl, optionally substituted C₂₋₃₀alkenyl, optionally substituted C₂₋₃₀alkynyl, optionally substituted C₁₋₃₀ alkoxy (e.g., methoxy), alkoxyalkyl (e.g., 2-methoxyethyl), alkoxyalkylamine, alkoxyoxycarboxylate, amino, alkylamino, dialkylamiino, 5-8 membered heterocyclyl, —O—C₄₋₃₀alkyl-ON(CH₂R⁸)(CH₂R⁹), —O—C₄₋₃₀alkyl-ON(CH₂R⁸)(CH₂R⁹), a ligand, a linker covalently bonded to one or more ligands, a solid support, a linker or a linker covalently bonded to a solid support.

In some embodiments of the any one of the aspects described herein, R³ is —O(CH₂)ₘ₁—X^{M′}—R^{M′}. For example, R³ is —O(CH₂)ₘ₁—O—R^{M′}. In another non-limiting example, R³ is —O(CH₂)ₘ₁—S—R^{M′}.

In some embodiments of the any one of the aspects described herein, R³ is —OCH₂CH₂—X^{M′}—R^{M′}. For example, R³ is —OCH₂CH₂—O—R^{M′} In another non-limiting example, R³ is —OCH₂CH₂—S—R^{M′}.

In some embodiments of the any one of the aspects described herein, R³ is —O(CH₂)ₙ₁—C(Y^{M})N(R^{N′})(R^{N″}). For example, R³ is —O(CH₂)ₙ₁—C(O)N(R^{N′})(R^{N″}). In some embodiments, R³ is —OCH₂—C(O)N(R^{N′})(R^{N″}). In some other embodiments, R³ is —OCH₂CH₂—C(O)N(R^{N′})(R^{N″}).

In some embodiments of the any one of the aspects described herein, R³ is hydrogen, hydroxyl, protected hydroxyl, halogen, optionally substituted C₁₋₃₀ alkoxy, a reactive phosphorous group, a solid support, a linker, or a linker covalently attached to a solid support. For example, wherein R³ is hydrogen, hydroxyl, protected hydroxyl, halogen, a reactive phosphorous group, a solid support, a linker, or a linker covalently attached to a solid support. In some embodiments of the any one of the aspects described herein, R³ is hydrogen, hydroxyl, protected hydroxyl, a reactive phosphorous group, a solid support, a linker, or a linker covalently attached to a solid support. For example, R³ is a reactive phosphorous group, a solid support, a linker, or a linker covalently attached to a solid support. In some embodiments of the any one of the aspects described herein, R³ is a reactive phosphorous or a linker covalently attached to a solid support. For example, R³ is a reactive phosphorous group (e.g., a phosphoramidite, such as [(2-cyanoethyl)-(N,N-diisopropyl)]-phosphoramidite or [(ß-thiobenzoylethyl)-(1-pyrrolidinyl)]-thiophosphoramidite.

It is noted that in compounds of Formula (I), only one of R² and R³ is reactive phosphorous group, a solid support, or a linker covalently bonded to a solid support.

Optionally, only one of R² and R³ is —O(CH₂)ₘ₁—X^{M′}—R^{M′} (e.g., —OCH₂CH₂—X^{M′}—R^{M′}) or —O(CH₂)ₙ₁—C(Y^{M})N(R^{N′})(R^{N″}). Accordingly, in some embodiments, one of R² and R³ is —O(CH₂)ₘ₁—X^{M′}—R^{M′} (e.g., —OCH₂CH₂—X^{M′}—R^{M′}) or —O(CH₂)ₙ₁—C(Y^{M})N(R^{N′})(R^{N″}), and the other of R² and R³ is hydrogen, hydroxyl, protected hydroxyl, halogen, optionally substituted C₁₋₃₀ alkoxy, a reactive phosphorous group, a solid support, a linker, or a linker covalently attached to a solid support. For example, one of R² and R³ is —O(CH₂)ₘ₁—X^{M′}—R^{M′} (e.g., —OCH₂CH₂—X^{M′}—R^{M′}) or —O(CH₂)ₙ₁—C(Y^{M})N(R^{N′})(R^{N″}), and the other of R² and R³ is hydrogen, hydroxyl, protected hydroxyl, halogen, a reactive phosphorous group, a solid support, a linker, or a linker covalently attached to a solid support.

In some embodiments of the any one of the aspects described herein, one of R² and R³ is —O(CH₂)ₘ₁—X^{M′}—R^{M′} (e.g., —OCH₂CH₂—X^{M′}—R^{M′}) or —O(CH₂)ₙ₁—C(Y^{M})N(R^{N′})(R^{N″}), and the other of R² and R³ is hydrogen, hydroxyl, protected hydroxyl, a reactive phosphorous group, a solid support, a linker, or a linker covalently attached to a solid support. For example, one of R² and R³ is —O(CH₂)ₘ₁—X^{M′}—R^{M′} (e.g., —OCH₂CH₂—X^{M′}—R^{M′}) or —O(CH₂)ₙ₁—C(Y^{M})N(R^{N′})(R^{N″}), and the other of R² and R³ is a reactive phosphorous group, a solid support, a linker, or a linker covalently attached to a solid support. In some embodiments of the any one of the aspects described herein, one of R² and R³ is —O(CH₂)ₘ₁—X^{M′}—R^{M′} (e.g., —OCH₂CH₂—X^{M′}—R^{M′}) or —O(CH₂)ₙ₁—C(Y^{M})N(R^{N′})(R^{N″}), and the other of R² and R³ is a reactive phosphorous or a linker covalently attached to a solid support. For example, one of R² and R³ is —O(CH₂)ₘ₁—X^{M′}—R^{M′} (e.g., —OCH₂CH₂—X^{M′}—R^{M′}) or —O(CH₂)ₙ₁—C(Y^{M})N(R^{N′})(R^{N″}), and the other of R² and R³ is a reactive phosphorous group (e.g., a phosphoramidite, such as [(2-cyanoethyl)-(N,N-diisopropyl)]-phosphoramidite or [(ß-thiobenzoylethyl)-(1-pyrrolidinyl)]-thiophosphoramidite.

In some embodiments of the any one of the aspects described herein, R² is —O(CH₂)ₘ₁—X^{M′}—R^{M′} (e.g., —OCH₂CH₂—X^{M′}—R^{M′}) or —O(CH₂)ₙ₁—C(Y^{M})N(R^{N′})(R^{N″}), and R³ is hydrogen, hydroxyl, protected hydroxyl, a reactive phosphorous group, a solid support, a linker, or a linker covalently attached to a solid support. For example, R² is —O(CH₂)ₘ₁—X^{M′}—R^{M′} (e.g., —OCH₂CH₂—X^{M′}—R^{M′}) or —O(CH₂)ₙ₁—C(Y^{M})N(R^{N′})(R^{N″}), and R³ is a reactive phosphorous group, a solid support, a linker, or a linker covalently attached to a solid support. In some embodiments of the any one of the aspects described herein, R² is —O(CH₂)ₘ₁—X^{M′}—R^{M′} (e.g., —OCH₂CH₂—X^{M′}—R^{M′}) or —O(CH₂)ₙ₁—C(Y^{M})N(R^{N′})(R^{N″}), and R³ is a reactive phosphorous or a linker covalently attached to a solid support. For example, R² is —O(CH₂)ₘ₁—X^{M′}—R^{M′} (e.g., —OCH₂CH₂—X^{M′}—R^{M′}) or —O(CH₂)ₙ₁—C(Y^{M})N(R^{N′})(R^{N″}), and R³ is a reactive phosphorous group (e.g., a phosphoramidite, such as [(2-cyanoethyl)-(N,N-diisopropyl)]-phosphoramidite or [(ß-thiobenzoylethyl)-(1-pyrrolidinyl)]-thiophosphoramidite.

In some embodiments of the any one of the aspects described herein, R³ is —O(CH₂)ₘ₁—X^{M′}—R^{M′} (e.g., —OCH₂CH₂—X^{M′}—R^{M′}) or —O(CH₂)ₙ₁—C(Y^{M})N(R^{N′})(R^{N″}), and R² is hydrogen, hydroxyl, protected hydroxyl, a reactive phosphorous group, a solid support, a linker, or a linker covalently attached to a solid support. For example, R³ is —O(CH₂)ₘ₁—X^{M′}—R^{M′} (e.g., —OCH₂CH₂—X^{M′}—R^{M′}) or —O(CH₂)ₙ₁—C(Y^{M})N(R^{N′})(R^{N″}), and R² is a reactive phosphorous group, a solid support, a linker, or a linker covalently attached to a solid support. In some embodiments of the any one of the aspects described herein, R³ is —O(CH₂)ₘ₁—X^{M′}—R^{M′} (e.g., —OCH₂CH₂—X^{M′}—R^{M′}) or —O(CH₂)ₙ₁—C(Y^{M})N(R^{N′})(R^{N″}), and R² is a reactive phosphorous or a linker covalently attached to a solid support. For example, R³ is —O(CH₂)ₘ₁—X^{M′}—R^{M′} (e.g., —OCH₂CH₂—X^{M′}—R^{M′}) or —O(CH₂)ₙ₁—C(Y^{M})N(R^{N′})(R^{N″}), and R² is a reactive phosphorous group (e.g., a phosphoramidite, such as [(2-cyanoethyl)-(N,N-diisopropyl)]-phosphoramidite or [(ß-thiobenzoylethyl)-(1-pyrrolidinyl)]-thiophosphoramidite.

In some embodiments of the various aspects described herein, R⁴ is H. In some other embodiments of the various aspects described herein, R⁴ is R^{M}. For example, R⁴ is —O(CH₂)ₘ₁—X^{M′}—R^{M′} (e.g., —OCH₂CH₂—X^{M′}—R^{M′}) or —O(CH₂)ₙ₁—C(Y^{M})N(R^{N′})(R^{N″}).

In compounds of Formula (I), R⁵ can be R^{MA}, hydrogen, hydroxyl, protected hydroxyl, optionally substituted C₁₋₃₀ alkyl, optionally substituted C₂₋₃₀alkenyl, optionally substituted C₂₋₃₀ alkynyl, optionally substituted C₁₋₃₀ alkoxy, optionally substituted 3-8 membered heterocyclyl (e.g., morpholin-1-yl, piperidin-1-yl, or pyrrolidin-1-yl), halogen, alkoxyalkyl (e.g., 2-methoxyethyl), alkoxyalkylamine, alkoxyoxycarboxylate, amino, alkylamino, dialkylamino, —O—C₄₋₃₀alkyl-ON(CH₂R⁸)(CH₂R⁹), —O—C₄₋₃₀alkyl-ON(CH₂R⁸)(CH₂R⁹), vinylphosphonate (VP) group (e.g., ═CH—X^{P}, X^{P} is a phosphate group), C₃₋₆ cycloalkylphosphonate (e.g., cyclopropylphosphonate), monophosphate ((HO)₂(O)P—O-5′), diphosphate ((HO)₂(O)P—O—P(HO)(O)—O-5′), triphosphate ((HO)₂(O)P—O—(HO)(O)P—O—P(HO)(O)—O-5′); monothiophosphate (phosphorothioate, (HO)₂(S)P—O-5′), monodithiophosphate (phosphorodithioate; (HO)(HS)(S)P—O-5′), phosphorothiolate ((HO)₂(O)P—S-5′); alpha-thiotriphosphate; beta-thiotriphosphate; gamma-thiotriphosphate; phosphoramidates ((HO)₂(O)P—NH-5′, (HO)(NH₂)(O)P—O-5′), alkylphosphonates [(R^{P})(OH)(O)P—O-5′, R^{P} is optionally substituted C₁₋₃₀ alkyl, e.g., methyl, ethyl, isopropyl, or propyl)], alkyletherphosphonates [(R^{P1})(OH)(O)P—O-5′, R^{P1} is alkoxyalkyl, e.g., methoxymethyl (CH₂OMe) or ethoxymethyl], (HO)₂(X)P—O[—(CH₂)ₐ—O—P(X)(OH)—O]_{b}-5′ or (HO)₂(X)P—O[—(CH₂)ₐ—P(X)(OH)—O]_{b}-5′ or (HO)₂(X)P—[—(CH₂)ₐ—O—P(X)(OH)—O]_{b}-5′, or optionally substituted alkyl, and dialkyl terminal phosphates and phosphate mimics (e.g., HO[—(CH₂)ₐ—O—P(X)(OH)—O]_{b}-5′, H₂N[—(CH₂)ₐ—O—P(X)(OH)—O]_{b}-5′, H[—(CH₂)ₐ—O—P(X)(OH)—O]_{b}-5′, Me₂N[—(CH₂)ₐ—O—P(X)(OH)—O]_{b}-5′, HO[—(CH₂)ₐ—P(X)(OH)—O]_{b}-5′, H2N[—(CH₂)ₐ—P(X)(OH)—O]_{b}-5′, H[—(CH₂)ₐ—P(X)(OH)—O]_{b}-5′, Me₂N[—(CH₂)ₐ—P(X)(OH)—O]_{b}-5′, wherein X is O or S; a and b are each independently 1-10; and each R¹ and R⁹ is independently H, a targeting ligand (e.g., GalNac), a pharmacokinetics modifier, optionally substituted C₁₋₃₀ alkyl, optionally substituted C₁₋₃₀ alkenyl, or optionally substituted C₁₋₃₀alkynyl.

In some embodiments of the any one of the aspects described herein, R⁵ is —O(CH₂)ₘ₁—X^{M′}—R^{M′}. For example, R⁵ is —O(CH₂)ₘ₁—O—R^{M′}. In another non-limiting example, R⁵ is —O(CH₂)ₘ₁—S—R^{M′}.

In some embodiments of the any one of the aspects described herein, R⁵ is —OCH₂CH₂—X^{M′}—R^{M′}. For example, R⁵ is —OCH₂CH₂—O—R^{M′} In another non-limiting example, R⁵ is —OCH₂CH₂—S—R^{M′}.

In some embodiments of the any one of the aspects described herein, R⁵ is —O(CH₂)ₙ₁—C(Y^{M})N(R^{N′})(R^{N″}). For example, —O(CH₂)ₙ₁—C(Y^{M})N(R^{N′})(R^{N″}). In some embodiments, R⁵ is —OCH₂—C(O)N(R^{N′})(R^{N″}). In some other embodiments, R⁵ is —OCH₂CH₂—C(O)N(R^{N′})(R^{N″}).

In some embodiments of any one of the aspects described herein, R⁵ is hydroxyl, protected hydroxyl, optionally substituted C₁₋₃₀ alkoxy, vinylphosphonate (VP) group, monophosphate, diphosphate, triphosphate, monothiophosphate (phosphorothioate), monodithiophosphate, phosphorothiolate, alpha-thiotriphosphate, beta-thiotriphosphate, gamma-thiotriphosphate, phosphoramidate, alkylphosphonate, alkyletherphosphonate, dialkyl terminal phosphate or phosphate mimic. For example, R⁵ is hydroxyl, protected hydroxyl, vinylphosphonate (VP) group, cyclopropylphosphonate, monophosphate, diphosphate, triphosphate, monothiophosphate (phosphorothioate), monodithiophosphate, phosphorothiolate, alpha-thiotriphosphate, beta-thiotriphosphate, gamma-thiotriphosphate, phosphoramidates, alkylphosphonate, alkyletherphosphonate, dialkyl terminal phosphate, or a phosphate mimic. In some embodiments of any one of the aspects described herein, R⁵ is hydroxyl or protected hydroxyl. In some other embodiments, of any one of the aspects described herein R⁵ is vinylphosphonate (VP) group.

In some embodiments of the any one of the aspects described herein, R² is —O(CH₂)ₘ₁—X^{M′}—R^{M′} (e.g., —OCH₂CH₂—X^{M′}—R^{M′}) or —O(CH₂)ₙ₁—C(O)N(R^{N′})(R^{N″}); R³ is hydrogen, hydroxyl, protected hydroxyl, a reactive phosphorous group, a solid support, a linker, or a linker covalently attached to a solid support; and R⁵ is hydroxyl, protected hydroxyl, optionally substituted C₁₋₃₀ alkoxy, vinylphosphonate (VP) group, monophosphate, diphosphate, triphosphate, monothiophosphate (phosphorothioate), monodithiophosphate, phosphorothiolate, alpha-thiotriphosphate, beta-thiotriphosphate, gamma-thiotriphosphate, phosphoramidate, alkylphosphonate, alkyletherphosphonate, dialkyl terminal phosphate or phosphate mimic. For example, R² is —O(CH₂)ₘ₁—X^{M′}—R^{M′} (e.g., —OCH₂CH₂—X^{M′}—R^{M′}) or —O(CH₂)ₙ₁—C(O)N(R^{N′})(R^{N″}); R³ is a reactive phosphorous group, a solid support, a linker, or a linker covalently attached to a solid support; and R⁵ is hydroxyl, protected hydroxyl, vinylphosphonate (VP) group, cyclopropylphosphonate, monophosphate, diphosphate, triphosphate, monothiophosphate (phosphorothioate), monodithiophosphate, phosphorothiolate, alpha-thiotriphosphate, beta-thiotriphosphate, gamma-thiotriphosphate, phosphoramidates, alkylphosphonate, alkyletherphosphonate, dialkyl terminal phosphate, or a phosphate mimic. In some embodiments of the any one of the aspects described herein, R² is —O(CH₂)ₘ₁—X^{M′}—R^{M′} (e.g., —OCH₂CH₂—X^{M′}—R^{M′}) or —O(CH₂)ₙ₁—C(O)N(R^{N′})(R^{N″}); R³ is a reactive phosphorous or a linker covalently attached to a solid support; and R⁵ is hydroxyl or protected hydroxyl. For example, R² is —O(CH₂)ₘ₁—X^{M′}—R^{M′} (e.g., —OCH₂CH₂—X^{M′}—R^{M′}) or —O(CH₂)ₙ₁—C(O)N(R^{N′})(R^{N″}); R³ is a reactive phosphorous group (e.g., a phosphoramidite, such as [(2-cyanoethyl)-(N,N-diisopropyl)]-phosphoramidite or [(ß-thiobenzoylethyl)-(1-pyrrolidinyl)]-thiophosphoramidite; and R⁵ is hydroxyl or protected hydroxyl. In another non-limiting example, R² is O(CH₂)ₘ₁—X^{M′}—R^{M′} (e.g., —OCH₂CH₂—X^{M′}—R^{M′}) or —O(CH₂)ₙ₁—C(O)N(R^{N′})(R^{N″}); R³ is a reactive phosphorous group (e.g., a phosphoramidite, such as [(2-cyanoethyl)-(N,N-diisopropyl)]-phosphoramidite or [(ß-thiobenzoylethyl)-(1-pyrrolidinyl)]-thiophosphoramidite; and R⁵ is a vinylphosphonate (VP) group. In yet another non-limiting example, R² is —O(CH₂)ₘ₁—X^{M′}—R^{M′} (e.g., —OCH₂CH₂—X^{M′}—R^{M′}) or —O(CH₂)ₙ₁—C(O)N(R^{N′})(R^{N″}); R³ is a linker covalently attached to a solid support; and R⁵ is hydroxyl or protected hydroxyl. In still yet another non-limiting example, R² is —O(CH₂)ₘ₁—X^{M′}-R^{M′} (e.g., —OCH₂CH₂—X^{M′}—R^{M′}) or —O(CH₂)ₙ₁—C(O)N(R^{N′})(R^{N″}); R³ is a linker covalently attached to a solid support; and R⁵ is a vinylphosphonate (VP) group.

In some embodiments of the any one of the aspects described herein, R³ is —O(CH₂)ₘ₁—X^{M′}—R^{M′} (e.g., —OCH₂CH₂—X^{M′}—R^{M′}) or —O(CH₂)ₙ₁—C(O)N(R^{N′})(R^{N″}); R² is hydrogen, hydroxyl, protected hydroxyl, a reactive phosphorous group, a solid support, a linker, or a linker covalently attached to a solid support; and R⁵ is hydroxyl, protected hydroxyl, optionally substituted C₁₋₃₀ alkoxy, vinylphosphonate (VP) group, monophosphate, diphosphate, triphosphate, monothiophosphate (phosphorothioate), monodithiophosphate, phosphorothiolate, alpha-thiotriphosphate, beta-thiotriphosphate, gamma-thiotriphosphate, phosphoramidate, alkylphosphonate, alkyletherphosphonate, dialkyl terminal phosphate or phosphate mimic. For example, R³ is —O(CH₂)ₘ₁—X^{M′}—R^{M′} (e.g., —OCH₂CH₂—X^{M′}—R^{M′}) or —O(CH₂)ₙ₁—C(O)N(R^{N′})(R^{N″}); R² is a reactive phosphorous group, a solid support, a linker, or a linker covalently attached to a solid support; and R⁵ is hydroxyl, protected hydroxyl, vinylphosphonate (VP) group, cyclopropylphosphonate, monophosphate, diphosphate, triphosphate, monothiophosphate (phosphorothioate), monodithiophosphate, phosphorothiolate, alpha-thiotriphosphate, beta-thiotriphosphate, gamma-thiotriphosphate, phosphoramidates, alkylphosphonate, alkyletherphosphonate, dialkyl terminal phosphate, or a phosphate mimic.

In some embodiments of the any one of the aspects described herein, R³ is —O(CH₂)ₘ₁—X^{M′}—R^{M′} (e.g., —OCH₂CH₂—X^{M′}—R^{M′}) or —O(CH₂)ₙ₁—C(O)N(R^{N′})(R^{N″}); R² is a reactive phosphorous or a linker covalently attached to a solid support; and R⁵ is hydroxyl or protected hydroxyl. For example, R³ is —O(CH₂)ₘ₁—X^{M′}—R^{M′} (e.g., —OCH₂CH₂—X^{M′}—R^{M′}) or —O(CH₂)ₙ₁—C(O)N(R^{N′})(R^{N″}); R² is a reactive phosphorous group (e.g., a phosphoramidite, such as [(2-cyanoethyl)-(N,N-diisopropyl)]-phosphoramidite or [(ß-thiobenzoylethyl)-(1-pyrrolidinyl)]-thiophosphoramidite; and R⁵ is hydroxyl or protected hydroxyl. In another non-limiting example, R³ is —O(CH₂)ₘ₁—X^{M′}—R^{M′} (e.g., —OCH₂CH₂—X^{M′}—R^{M′}) or —O(CH₂)ₙ₁—C(O)N(R^{N′})(R^{N″}); R² is a reactive phosphorous group (e.g., a phosphoramidite, such as [(2-cyanoethyl)-(N,N-diisopropyl)]-phosphoramidite or [(ß-thiobenzoylethyl)-(1-pyrrolidinyl)]-thiophosphoramidite; and R⁵ is a vinylphosphonate (VP) group. In yet another non-limiting example, R³ is —O(CH₂)ₘ₁—X^{M′}—R^{M′} (e.g., —OCH₂CH₂—X^{M′}—R^{M′}) or —O(CH₂)ₙ₁—C(O)N(R^{N′})(R^{N″}); R² is a linker covalently attached to a solid support; and R⁵ is hydroxyl or protected hydroxyl. In still yet another non-limiting example, R³ is —O(CH₂)ₘ₁—X^{M′}—R^{M′} (e.g., —OCH₂CH₂—X^{M′}—R^{M′}) or —O(CH₂)ₙ₁—C(O)N(R^{N′})(R^{N″}); R² is a linker covalently attached to a solid support; and R⁵ is a vinylphosphonate (VP) group.

In some compounds of Formula (I), when R² is —O(CH₂)ₘ₁—X^{M′}—R^{M′} (e.g., —OCH₂CH₂—X^{M′}—R^{M′}); R⁵ is hydroxyl or protected hydroxyl; R⁴ is H; and R³ is hydroxyl, protected hydroxyl, a phosphate group or a reactive phosphorous group, then R^{M′} is not unsubstituted C₆₋₂₁ alkyl, unsubstituted C₆₋₂₁alkenyl, or unsubstituted C₆₋₂₁alkynyl.

In some compounds of Formula (I), when R² is —O(CH₂)ₙ₁—C(O)N(R^{N′})(R^{N″}); n1 is 1; R⁵ is hydroxyl or protected hydroxyl; R⁴ is H; and R³ is hydroxyl, protected hydroxyl, a phosphate group or a reactive phosphorous group; and one of R^{N′} and R^{N″} is H, then the other of R^{N′} and R^{N″} is not a substituted or unsubstituted C₅₋₈alkyl. For example, when R² is —O(CH₂)ₙ₁—C(O)N(R^{N′})(R^{N″}); n1 is 1; R⁵ is hydroxyl or protected hydroxyl; R⁴ is H; and R³ is hydroxyl, protected hydroxyl, a phosphate group or a reactive phosphorous group; and one of R^{N′} and R^{N″} is H, then the other of R^{N′} and R^{N″} is not —(CH₂)₆CH₃, —(CH₂)₇CH₃, —(CH₂)₈CH₃, —(CH₂)₅NHCOCF₃, —(CH₂)₆NHCOCF₃, —(CH₂)₇NHCOCF₃, —(CH₂)₅N(CH₃)₂, —(CH₂)₆N(CH₃)₂ or —(CH₂)₇N(CH₃)₂.

Certain compounds of Formula (I) are useful for preparing oligonucleotides. Accordingly, in another aspect, provided herein is an oligonucleotide prepared using a compound of Formula (I). For example, an oligonucleotide comprising at least one nucleotide of Formula (II).

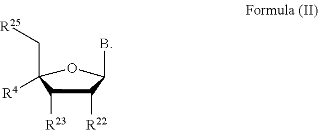

In nucleotides of Formula (II), one of R²², R²³, R⁴ and R²⁵ is R^{MA}. Optionally, only one of R²², R²³, R⁴ and R²⁵ is R^{MA}. Accordingly, in some embodiments of the various aspects described herein, one and only one of R²², R²³, R⁴ and R²⁵ is R^{MA}.

In nucleotides of Formula (II), B is an optionally modified nucleobase. For example, B can be natural or non-natural nucleobase, each of which can be optionally modified with one or more of functional groups, ligands, protecting groups and the like. In some embodiments, B is an unmodified nucleobase. In some other embodiments, B is a modified nucleobase.

In nucleotides of Formula (II), R²² is R^{MA}, a bond to an internucleotide linkage to a subsequent nucleotide, hydrogen, hydroxyl, protected hydroxyl, halogen, optionally substituted C₁₋₃₀ alkyl, optionally substituted C₂₋₃₀alkenyl, optionally substituted C₂₋₃₀alkynyl, optionally substituted C₁₋₃₀ alkoxy (e.g., methoxy), alkoxyalkyl (e.g., 2-methoxyethyl), alkoxyalkylamine, alkoxyoxycarboxylate, amino, alkylamino, dialkylamino, 5-8 membered heterocyclyl, —O—C₄₋₃₀alkyl-ON(CH₂R⁸)(CH₂R⁹), —O—C₄₋₃₀alkyl-ON(CH₂R⁸)(CH₂R⁹), a ligand, a linker covalently bonded to one or more ligands, a solid support, a linker or a linker covalently bonded to a solid support.

In some embodiments of the any one of the aspects described herein, R²² is —O(CH₂)ₘ₁—X^{M′}—R^{M′}. For example, R²² is —O(CH₂)ₘ₁—O—R^{M′}. In another non-limiting example, R²² is —O(CH₂)ₘ₁—S—R^{M′}.

In some embodiments of the any one of the aspects described herein, R²² is —OCH₂CH₂—X^{M′}—R^{M′}. For example, R²² is —OCH₂CH₂—O—R^{M′} In another non-limiting example, R²² is —OCH₂CH₂—S—R^{M′}.

In some embodiments of the any one of the aspects described herein, R²² is —O(CH₂)ₙ₁—C(Y^{M})N(R^{N′})(R^{N″}). For example, R²² is —O(CH₂)ₙ₁—C(O)N(R^{N′})(R^{N″}). In some embodiments, R²² is —OCH₂—C(O)N(R^{N′})(R^{N″}). In some other embodiments, R²² is —OCH₂CH₂—C(O)N(R^{N′})(R^{N″}).

In some embodiments of any one of the aspects described herein, R²² is a bond to an internucleotide linkage to a subsequent nucleotide, hydrogen, hydroxyl, protected hydroxyl, halogen, optionally substituted C₁₋₃₀ alkoxy, a solid support, a linker, or a linker covalently attached to a solid support. For example, R²² is a bond to an internucleotide linkage to a subsequent nucleotide, hydroxyl, protected hydroxyl, a solid support, a linker, or a linker covalently attached to a solid support. In some embodiments of any one of the aspects described herein, R²² is a bond to an internucleotide linkage to a subsequent nucleotide. In some embodiments of any one of the aspects described herein, R²² is a linker covalently attached to a solid support. In some embodiments of any one of the aspects described herein, R²² is hydroxyl or protected hydroxyl.

In nucleotides of Formula (II), R²³ is R^{MA}, a bond to an internucleotide linkage to a subsequent nucleotide, hydrogen, hydroxyl, protected hydroxyl, halogen, optionally substituted C₁₋₃₀ alkyl, optionally substituted C₂₋₃₀alkenyl, optionally substituted C₂₋₃₀alkynyl, optionally substituted C₁₋₃₀ alkoxy (e.g., methoxy), alkoxyalkyl (e.g., 2-methoxyethyl), alkoxyalkylamine, alkoxyoxycarboxylate, amino, alkylamino, dialkylamino, 5-8 membered heterocyclyl, —O—C₄₋₃₀alkyl-ON(CH₂R⁸)(CH₂R⁹), —O—C₄₋₃₀alkyl-ON(CH₂R⁸)(CH₂R⁹), a ligand, a linker covalently bonded to one or more ligands, a solid support, a linker or a linker covalently bonded to a solid support.

In some embodiments of the any one of the aspects described herein, R²³ is —O(CH₂)ₘ₁—X^{M′}—R^{M′}. For example, R²³ is —O(CH₂)ₘ₁—O—R^{M′}. In another non-limiting example, R²³ is —O(CH₂)ₘ₁—S—R^{M′}.

In some embodiments of the any one of the aspects described herein, R²³ is —OCH₂CH₂—X^{M′}—R^{M′}. For example, R²³ is —OCH₂CH₂—O—R^{M′} In another non-limiting example, R²³ is —OCH₂CH₂—S—R^{M′}.

In some embodiments of the any one of the aspects described herein, R²³ is —O(CH₂)ₙ₁—C(Y^{M})N(R^{N′})(R^{N″}). For example, R²³ is —O(CH₂)ₙ₁—C(O)N(R^{N′})(R^{N″}). In some embodiments, R²³ is —OCH₂—C(O)N(R^{N′})(R^{N″}). In some other embodiments, R²³ is —OCH₂CH₂—C(O)N(R^{N′})(R^{N″}).

In some embodiments of any one of the aspects described herein, R²³ is a bond to an internucleotide linkage to a subsequent nucleotide, hydrogen, hydroxyl, protected hydroxyl, halogen, optionally substituted C₁₋₃₀alkoxy, a solid support, a linker, or a linker covalently attached to a solid support. For example, R²³ is a bond to an internucleotide linkage to a subsequent nucleotide, hydroxyl, protected hydroxyl, a solid support, a linker, or a linker covalently attached to a solid support. In some embodiments of any one of the aspects described herein, R²³ is a bond to an internucleotide linkage to a subsequent nucleotide. In some embodiments of any one of the aspects described herein, R²³ is a linker covalently attached to a solid support. In some embodiments of any one of the aspects described herein, R²³ is hydroxyl or protected hydroxyl.

It is noted that in nucleotides of Formula (II), only one of R²² and R²³ is a bond to an internucleotide linkage to a subsequent nucleotide, a solid support, or a linker covalently bonded to a solid support.

Accordingly, in some embodiments of any one of the aspects described herein, one of R²² and R²³ is —O(CH₂)ₘ₁—X^{M′}—R^{M′} (e.g., —OCH₂CH₂—X^{M′}—R^{M′}) or —O(CH₂)ₙ₁—C(Y^{M})N(R^{N′})(R^{N″}) and the other one of R²² and R²³ is a bond to an internucleotide linkage to a subsequent nucleotide, hydrogen, hydroxyl, protected hydroxyl, halogen, optionally substituted C₁₋₃₀ alkoxy, a solid support, a linker, or a linker covalently attached to a solid support. For example, one of R²² and R²³ is —O(CH₂)ₘ₁—X^{M′}—R^{M′} (e.g., —OCH₂CH₂—X^{M′}—R^{M′}) or —O(CH₂)ₙ₁—C(Y^{M})N(R^{N′})(R^{N″}) and the other one of R²² and R²³ is a bond to an internucleotide linkage to a subsequent nucleotide, hydroxyl, protected hydroxyl, or a linker covalently attached to a solid support. In some embodiments, one of R²² and R²³ is —O(CH₂)ₘ₁—X^{M′}—R^{M′} (e.g., —OCH₂CH₂—X^{M′}—R^{M′}) or —O(CH₂)ₙ₁—C(Y^{M})N(R^{N′})(R^{N″}) and the other one of R²² and R²³ is a bond to an internucleotide linkage to a subsequent nucleotide. In some other embodiments, one of R²² and R²³ is —O(CH₂)ₘ₁—X^{M′}—R^{M′} (e.g., —OCH₂CH₂—X^{M′}—R^{M′}) or —O(CH₂)ₙ₁—C(Y^{M})N(R^{N′})(R^{N″}) and the other one of R²² and R²³ is hydroxyl, protected hydroxyl or a linker covalently attached to a solid support.

In some embodiments of any the aspects described herein, R²² is —O(CH₂)ₘ₁—X^{M′}—R^{M′} (e.g., —OCH₂CH₂—X^{M′}—R^{M′}) or —O(CH₂)ₙ₁—C(Y^{M})N(R^{N′})(R^{N″}) and R²³ is a bond to an internucleotide linkage to a subsequent nucleotide, hydrogen, hydroxyl, protected hydroxyl, halogen, optionally substituted C₁₋₃₀ alkoxy, a solid support, a linker, or a linker covalently attached to a solid support. For example, R²² is —O(CH₂)ₘ₁—X^{M′}—R^{M′} (e.g., —OCH₂CH₂—X^{M′}—R^{M′}) or —O(CH₂)ₙ₁—C(Y^{M})N(R^{N′})(R^{N″}) and R²³ is a bond to an internucleotide linkage to a subsequent nucleotide, hydroxyl, protected hydroxyl, or a linker covalently attached to a solid support. In some embodiments, R²² is —O(CH₂)ₘ₁—X^{M′}—R^{M′} (e.g., —OCH₂CH₂—X^{M′}—R^{M′}) or —O(CH₂)ₙ₁—C(Y^{M})N(R^{N′})(R^{N″}) and R²³ is a bond to an internucleotide linkage to a subsequent nucleotide. In some other embodiments, R²² is —O(CH₂)ₘ₁—X^{M′}—R^{M′} (e.g., —OCH₂CH₂—X^{M′}—R^{M′}) or —O(CH₂)ₙ₁—C(Y^{M})N(R^{N′})(R^{N″}) and R²³ is hydroxyl, protected hydroxyl or a linker covalently attached to a solid support.

In some embodiments of any the aspects described herein, R²³ is —O(CH₂)ₘ₁—X^{M′}—R^{M′} (e.g., —OCH₂CH₂—X^{M′}—R^{M′}) or —O(CH₂)ₙ₁—C(Y^{M})N(R^{N′})(R^{N″}) and R²² is a bond to an internucleotide linkage to a subsequent nucleotide, hydrogen, hydroxyl, protected hydroxyl, halogen, optionally substituted C₁₋₃₀ alkoxy, a solid support, a linker, or a linker covalently attached to a solid support. For example, R²³ is —O(CH₂)ₘ₁—X^{M′}—R^{M′} (e.g., —OCH₂CH₂—X^{M′}—R^{M′}) or —O(CH₂)ₙ₁—C(Y^{M})N(R^{N′})(R^{N″}) and R²² is a bond to an internucleotide linkage to a subsequent nucleotide, hydroxyl, protected hydroxyl, or a linker covalently attached to a solid support. In some embodiments, R²³ is —O(CH₂)ₘ₁—X^{M′}—R^{M′} (e.g., —OCH₂CH₂—X^{M′}—R^{M′}) or —O(CH₂)ₙ₁—C(Y^{M})N(R^{N′})(R^{N″}) and R²² is a bond to an internucleotide linkage to a subsequent nucleotide. In some other embodiments, R²³ is —O(CH₂)ₘ₁—X^{M′}—R^{M′} (e.g., —OCH₂CH₂—X^{M′}—R^{M′}) or —O(CH₂)ₙ₁—C(Y^{M})N(R^{N′})(R^{N″}) and R²² is hydroxyl, protected hydroxyl or a linker covalently attached to a solid support.

In nucleotides of Formula (II), R²⁵ can be a bond to an internucleotide linkage to a preceding nucleotide, hydroxyl, protected hydroxyl, optionally substituted C₁₋₃₀ alkoxy, vinylphosphonate (VP) group, monophosphate, diphosphate, triphosphate, monothiophosphate (phosphorothioate), monodithiophosphate, phosphorothiolate, alpha-thiotriphosphate, beta-thiotriphosphate, gamma-thiotriphosphate, phosphoramidate, alkylphosphonate, alkyletherphosphonate, dialkyl terminal phosphate or phosphate mimic. For example, R²⁵ is a bond to an internucleotide linkage to a preceding nucleotide, hydroxyl, protected hydroxyl, vinylphosphonate (VP) group, cyclopropylphosphonate, monophosphate, diphosphate, triphosphate, monothiophosphate (phosphorothioate), monodithiophosphate, phosphorothiolate, alpha-thiotriphosphate, beta-thiotriphosphate, gamma-thiotriphosphate, phosphoramidates, alkylphosphonate, alkyletherphosphonate, dialkyl terminal phosphate, or a phosphate mimic.

In some embodiments of any one of the aspects described herein, R²⁵ is a bond to an internucleotide linkage to a preceding nucleotide.

In some embodiments of any one of the aspects described herein, R²⁵ is hydroxyl or protected hydroxyl.

In some embodiments of any one of the aspects described herein, R²⁵ is a vinylphosphonate (VP) group, cyclopropylphosphonate, monophosphate, diphosphate, triphosphate, monothiophosphate (phosphorothioate), monodithiophosphate, phosphorothiolate, alpha-thiotriphosphate, beta-thiotriphosphate, gamma-thiotriphosphate, phosphoramidates, alkylphosphonate, alkyletherphosphonate, dialkyl terminal phosphate, or a phosphate mimic. For example, R²⁵ is a vinylphosphonate (VP) group.

It is noted that when both of R²² and R²³ are not bond to an internucleotide linkage to a subsequent nucleotide, then R²⁵ is a bond to an internucleotide linkage to a preceding nucleotide.

In some embodiments of any the aspects described herein, R²² is —O(CH₂)ₘ₁—X^{M′}—R^{M′} (e.g., —OCH₂CH₂—X^{M′}—R^{M′}) or —O(CH₂)ₙ₁—C(Y^{M})N(R^{N′})(R^{N″}); R²³ is a bond to an internucleotide linkage to a subsequent nucleotide, hydrogen, hydroxyl, protected hydroxyl, halogen, a solid support, a linker, or a linker covalently attached to a solid support; and R²⁵ is a bond to an internucleotide linkage to a preceding nucleotide, hydroxyl, protected hydroxyl, vinylphosphonate (VP) group, cyclopropylphosphonate, monophosphate, diphosphate, triphosphate, monothiophosphate (phosphorothioate), monodithiophosphate, phosphorothiolate, alpha-thiotriphosphate, beta-thiotriphosphate, gamma-thiotriphosphate, phosphoramidates, alkylphosphonate, alkyletherphosphonate, dialkyl terminal phosphate, or a phosphate mimic, provided that R²³ is a bond to an internucleotide linkage to a subsequent nucleotide or R²⁵ is a bond to an internucleotide linkage to a preceding nucleotide.

In some embodiments, R²² is —O(CH₂)ₘ₁—X^{M′}—R^{M′} (e.g., —OCH₂CH₂—X^{M′}—R^{M′}) or —O(CH₂)ₙ₁—C(Y^{M})N(R^{N′})(R^{N″}); R²³ is a bond to an internucleotide linkage to a subsequent nucleotide; and R²⁵ is hydroxyl, protected hydroxyl, vinylphosphonate (VP) group, cyclopropylphosphonate, monophosphate, diphosphate, triphosphate, monothiophosphate (phosphorothioate), monodithiophosphate, phosphorothiolate, alpha-thiotriphosphate, beta-thiotriphosphate, gamma-thiotriphosphate, phosphoramidates, alkylphosphonate, alkyletherphosphonate, dialkyl terminal phosphate, or a phosphate mimic. For example, R²² is —O(CH₂)ₘ₁—X^{M′}—R^{M′} (e.g., —OCH₂CH₂—X^{M′}—R^{M′})′ or —O(CH₂)ₙ₁—C(Y^{M})N(R^{N′})(R^{N″}); R²³ is a bond to an internucleotide linkage to a subsequent nucleotide; and R²⁵ is hydroxyl, protected hydroxyl or vinylphosphonate (VP) group.

In some embodiments, R²² is —O(CH₂)ₘ₁—X^{M′}—R^{M′} (e.g., —OCH₂CH₂—X^{M′}—R^{M′}) or —O(CH₂)ₙ₁—C(O)N(R^{N′})(R^{N″}); R²³ is a bond to an internucleotide linkage to a subsequent nucleotide; and R²⁵ is a bond to an internucleotide linkage to a preceding nucleotide.

In some embodiments of any the aspects described herein, R²² is —O(CH₂)ₘ₁—X^{M′}—R^{M′} (e.g., —OCH₂CH₂—X^{M′}—R^{M′}) or —O(CH₂)ₙ₁—C(O)N(R^{N′})(R^{N″}); R²³ is hydroxyl, protected hydroxyl, a solid support, a linker, or a linker covalently attached to a solid support; and R²⁵ is a bond to an internucleotide linkage to a preceding nucleotide. For example, R²² is —OCH₂CH₂—X^{M′}—R^{M′} or —O(CH₂)ₙ₁—C(O)N(R^{N′})(R^{N″}); R²³ is hydroxyl, protected hydroxyl or a linker covalently attached to a solid support.

In some embodiments of any the aspects described herein, R²³ is —O(CH₂)ₘ₁—X^{M′}—R^{M′} (e.g., —OCH₂CH₂—X^{M′}—R^{M′}) or —O(CH₂)ₙ₁—C(Y^{M})N(R^{N′})(R^{N″}); R²² is a bond to an internucleotide linkage to a subsequent nucleotide, hydrogen, hydroxyl, protected hydroxyl, halogen, a solid support, a linker, or a linker covalently attached to a solid support; and R²⁵ is a bond to an internucleotide linkage to a preceding nucleotide, hydroxyl, protected hydroxyl, vinylphosphonate (VP) group, cyclopropylphosphonate, monophosphate, diphosphate, triphosphate, monothiophosphate (phosphorothioate), monodithiophosphate, phosphorothiolate, alpha-thiotriphosphate, beta-thiotriphosphate, gamma-thiotriphosphate, phosphoramidates, alkylphosphonate, alkyletherphosphonate, dialkyl terminal phosphate, or a phosphate mimic, provided that R²² is a bond to an internucleotide linkage to a subsequent nucleotide or R²⁵ is a bond to an internucleotide linkage to a preceding nucleotide.

In some embodiments, R²³ is —O(CH₂)ₘ₁—X^{M′}—R^{M′} (e.g., —OCH₂CH₂—X^{M′}—R^{M′}) or —O(CH₂)ₙ₁—C(Y^{M})N(R^{N′})(R^{N″}); R²² is a bond to an internucleotide linkage to a subsequent nucleotide; and R²⁵ is hydroxyl, protected hydroxyl, vinylphosphonate (VP) group, cyclopropylphosphonate, monophosphate, diphosphate, triphosphate, monothiophosphate (phosphorothioate), monodithiophosphate, phosphorothiolate, alpha-thiotriphosphate, beta-thiotriphosphate, gamma-thiotriphosphate, phosphoramidates, alkylphosphonate, alkyletherphosphonate, dialkyl terminal phosphate, or a phosphate mimic. For example, R²³ is —O(CH₂)ₘ₁—X^{M′}—R^{M′} (e.g., —OCH₂CH₂—X^{M′}—R^{M′}) or —O(CH₂)ₙ₁—C(Y^{M})N(R^{N′})(R^{N″}); R²² is a bond to an internucleotide linkage to a subsequent nucleotide; and R²⁵ is hydroxyl, protected hydroxyl or vinylphosphonate (VP) group.

In some embodiments, R²³ is —O(CH₂)ₘ₁—X^{M′}—R^{M′} (e.g., —OCH₂CH₂—X^{M′}—R^{M′}) or —O(CH₂)ₙ₁—C(O)N(R^{N′})(R^{N″}); R²² is a bond to an internucleotide linkage to a subsequent nucleotide; and R²⁵ is a bond to an internucleotide linkage to a preceding nucleotide.

In some embodiments of any the aspects described herein, R²³ is —O(CH₂)ₘ₁—X^{M′}—R^{M′} (e.g., —OCH₂CH₂—X^{M′}—R^{M′}) or —O(CH₂)ₙ₁—C(O)N(R^{N′})(R^{N″}); R²² is hydroxyl, protected hydroxyl, a solid support, a linker, or a linker covalently attached to a solid support; and R²⁵ is a bond to an internucleotide linkage to a preceding nucleotide. For example, R²³ is —O(CH₂)ₘ₁—X^{M′}—R^{M′} (e.g., —OCH₂CH₂—X^{M′}—R^{M′}) or —O(CH₂)ₙ₁—C(O)N(R^{N′})(R^{N″}); R²² is hydroxyl, protected hydroxyl or a linker covalently attached to a solid support.

In some embodiments of any the aspects described herein, R²⁵ is —O(CH₂)ₘ₁—X^{M′}—R^{M′} (e.g., —OCH₂CH₂—X^{M′}—R^{M′}) or —O(CH₂)ₙ₁—C(Y^{M})N(R^{N′})(R^{N″}) and one of R²² and R²³ is a bond to an internucleotide linkage to a subsequent nucleotide. For example, R²⁵ is O(CH₂)ₘ₁—X^{M′}—R^{M′} (e.g., —OCH₂CH₂—X^{M′}—R^{M′}) or —O(CH₂)ₙ₁—C(Y^{M})N(R^{N′})(R^{N″}) and R²³ is a bond to an internucleotide linkage to a subsequent nucleotide. In another non-limiting example, R²⁵ is —O(CH₂)ₘ₁—X^{M′}—R^{M′} (e.g., —OCH₂CH₂—X^{M′}—R^{M′}) or —O(CH₂)ₙ₁—C(Y^{M})N(R^{N′})(R^{N″}) and R²² is a bond to an internucleotide linkage to a subsequent nucleotide.

Optionally, in nucleotides of Formula (II), when R²² —O(CH₂)ₘ₁—X^{M′}—R^{M′} (e.g., —OCH₂CH₂—X^{M′}—R^{M′}); R²⁵ is hydroxyl, protected hydroxyl or a bond to a bond to an internucleotide linkage to a preceding nucleotide; R⁴ is H; R²³ is hydroxyl, protected hydroxyl, a bond to an internucleotide linkage to a subsequent nucleotide, a solid support, a linker, or a linker covalently attached to a solid support, and at least one of R²³ and R²⁵ is a bond to an internucleotide linkage, then R^{M′} is not an unsubstituted C₅₋₂₁ alkyl, unsubstituted C₅₋₂₁alkenyl, or unsubstituted C₅-21alkynyl.

Optionally, in nucleotides of Formula (II), when R²² is —O(CH₂)ₙ₁—C(O)N(R^{N′})(R^{N″}); n1 is 1; R⁵ hydroxyl, protected hydroxyl or a bond to a bond to an internucleotide linkage to a preceding nucleotide; R⁴ is H; R³ is hydroxyl, protected hydroxyl, a bond to an internucleotide linkage to a subsequent nucleotide, a solid support, a linker, or a linker covalently attached to a solid support, and at least one of R²³ and R²⁵ is a bond to an internucleotide linkage; one of R^{N′} and R^{N″} is H, and at least one of R²³ and R²⁵ is a bond to an internucleotide linkage, and then the other of R^{N′} and R^{N″} is not —(CH₂)₆CH₃, —(CH₂)₇CH₃, —(CH₂)₈CH₃, —(CH₂)₅NHCOCF₃, —(CH₂)₆NHCOCF₃, —(CH₂)₇NHCOCF₃, —(CH₂)₅N(CH₃)₂, —(CH₂)₆N(CH₃)₂ or —(CH₂)₇N(CH₃)₂.

In another aspect, provided herein is a method for preparing an oligonucleotide comprising at least one nucleotide of Formula (II), where one of R², R³, R⁴ and R⁵ is —O(CH₂)ₙ₁—C(Y^{M})N(R^{N′})(R^{N″}). The method comprising reacting an oligonucleotide comprising nucleotide of Formula (II′):

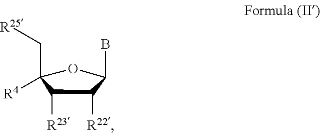

with an amine of formula HN(R^{N′})(R^{N″}).

In nucleotides of Formula (II′), B is an optionally modified nucleobase; R^{22′} is —O(CH₂)ₙ₁—C(Y^{M})OR^{LV}, a bond to an internucleotide linkage to a subsequent nucleotide, hydrogen, hydroxyl, protected hydroxyl, halogen, optionally substituted C₁₋₃₀ alkyl, optionally substituted C₂₋₃₀alkenyl, optionally substituted C₂₋₃₀alkynyl, optionally substituted C₁₋₃₀ alkoxy (e.g., methoxy), alkoxyalkyl (e.g., 2-methoxyethyl), alkoxyalkylamine, alkoxyoxycarboxylate, amino, alkylamino, dialkylamino, 5-8 membered heterocyclyl, —O—C₄₋₃₀alkyl-ON(CH₂R⁸)(CH₂R⁹), —O—C₄₋₃₀alkyl-ON(CH₂R⁸)(CH₂R⁹), a ligand, a linker covalently bonded to one or more ligands, a solid support, a linker or a linker covalently bonded to a solid support; R^{23′} is —O(CH₂)ₙ₁—C(Y^{M})OR^{LV}, a bond to an internucleotide linkage to a subsequent nucleotide, hydrogen, hydroxyl, protected hydroxyl, halogen, optionally substituted C₁₋₃₀ alkyl, optionally substituted C₂₋₃₀alkenyl, optionally substituted C₂₋₃₀alkynyl, optionally substituted C₁₋₃₀ alkoxy (e.g., methoxy), alkoxyalkyl (e.g., 2-methoxyethyl), alkoxyalkylamine, alkoxyoxycarboxylate, amino, alkylamino, dialkylamino, 5-8 membered heterocyclyl, —O—C₄₋₃₀alkyl-ON(CH₂R⁸)(CH₂R⁹), —O—C₄₋₃₀alkyl-ON(CH₂R⁸)(CH₂R⁹), a ligand, a linker covalently bonded to one or more ligands, a solid support, a linker or a linker covalently bonded to a solid support; R^{25′} is —O(CH₂)ₙ₁—C(O)OR^{LV}, a bond to an internucleotide linkage to a preceding nucleotide, hydrogen, hydroxyl, protected hydroxyl, optionally substituted C₁₋₃₀ alkyl, optionally substituted C₂₋₃₀alkenyl, optionally substituted C₂₋₃₀alkynyl, optionally substituted C₁₋₃₀ alkoxy, optionally substituted 3-8 membered heterocyclyl (e.g., morpholin-1-yl, piperidin-1-yl, or pyrrolidin-1-yl), halogen, alkoxyalkyl (e.g., 2-methoxyethyl), alkoxyalkylamine, alkoxyoxycarboxylate, amino, alkylamino, dialkylamino, —O—C₄₋₃₀alkyl-ON(CH₂R⁸)(CH₂R⁹), —O—C₄₋₃₀alkyl-ON(CH₂R⁸)(CH₂R⁹), vinylphosphonate (VP) group (e.g., ═CH—X^{P}, X^{P} is a phosphate group), C₃₋₆ cycloalkylphosphonate (e.g., cyclopropylphosphonate), monophosphate ((HO)₂(O)P—O-5′), diphosphate ((HO)₂(O)P—O—P(HO)(O)—O-5′), triphosphate ((HO)₂(O)P—O—(HO)(O)P—O—P(HO)(O)—O-5′); monothiophosphate (phosphorothioate, (HO)₂(S)P—O-5′), monodithiophosphate (phosphorodithioate; (HO)(HS)(S)P—O-5′), phosphorothiolate ((HO)₂(O)P—S-5′); alpha-thiotriphosphate; beta-thiotriphosphate; gamma-thiotriphosphate; phosphoramidates ((HO)₂(O)P—NH-5′, (HO)(NH2)(O)P—O-5′), alkylphosphonates [(R^{P})(OH)(O)P—O-5′, R^{P} is optionally substituted C₁₋₃₀ alkyl, e.g., methyl, ethyl, isopropyl, or propyl)], alkyletherphosphonates [(R^{P1})(OH)(O)P—O-5′, R^{P1} is alkoxyalkyl, e.g., methoxymethyl (CH₂OMe) or ethoxymethyl], (HO)₂(X)P—O[—(CH₂)ₐ—O—P(X)(OH)—O]_{b}-5′ or (HO)₂(X)P—O[—(CH₂)ₐ—P(X)(OH)—O]_{b}-5′ or (HO)₂(X)P—[—(CH₂)ₐ—O—P(X)(OH)—O]_{b}-5′, or optionally substituted alkyl, and dialkyl terminal phosphates and phosphate mimics (e.g., HO[—(CH₂)ₐ—O—P(X)(OH)—O]_{b}-5′, H2N[—(CH₂)ₐ—O—P(X)(OH)—O]_{b}-5′, H[—(CH₂)ₐ—O—P(X)(OH)—O]_{b}-5′, Me2N[—(CH₂)ₐ—O—P(X)(OH)—O]_{b}-5′, HO[—(CH₂)ₐ—P(X)(OH)—O]_{b}-5′, H₂N[—(CH₂)ₐ—P(X)(OH)—O]_{b}-5′, H[—(CH₂)ₐ—P(X)(OH)—O]_{b}-5′, Me₂N[—(CH₂)ₐ—P(X)(OH)—O]_{b}-5′, (wherein X is O or S; and a and b are each independently 1-10); R⁴ is hydrogen, optionally substituted C₁₋₆alkyl, optionally substituted C₂₋₆alkenyl, optionally substituted C₂₋₆alkynyl, or optionally substituted C₁₋₆alkoxy; R^{LV} is a C₁-C₆alkyl (e.g., ethyl); and each R⁸ and R⁹ is independently H, a targeting ligand (e.g., GalNac), a pharmacokinetics modifier, optionally substituted C₁₋₃₀ alkyl, optionally substituted C₁₋₃₀ alkenyl, or optionally substituted C₁₋₃₀alkynyl.

In some embodiments of the any one of the aspects described herein, R^{22′} is —O—R^{LV}.

In some embodiments of the any one of the aspects described herein, R^{23′} is —O—R^{LV}.

In some embodiments of the any one of the aspects described herein, R^{24′} is —O—R^{LV}.

In some embodiments of the any one of the aspects described herein, R^{25′} is —O—R^{LV}.

In some embodiments of the any one of the aspects described herein, the oligonucleotide comprising the nucleoside of Formula (II′) is linked to a solid support. For example, the olignucleotide comprising the nucleoside of Formula (II′) is reacted with the amine while the oligonucleotide is still attached to the solid support.

In some embodiments of the any one of the aspects described herein, the oligonucleotide comprising the nucleoside of Formula (II′) is linked to a solid support. For example, method comprises a step of cleaving the oligonucleotide from the solid support prior to reacting with the amine.

In some embodiments of any one of the aspects described herein, the oligonucleotide comprising the nucleoside of Formula (II′) comprises at least one modified internucleoside linkage.

In some embodiments of any one of the aspects described herein, the oligonucleotide comprising a nucleotide of Formula (II′) comprises at least one hydroxyl, phosphate or amino protecting group. For example, the olignucleotide comprising the nucleoside of Formula (II′) is reacted with the amine while the oligonucleotide comprises at least one hydroxyl, phosphate or amino protecting group.

In some embodiments of any one of the aspects described herein, the oligonucleotide comprising a nucleotide of Formula (II′) comprises at least one hydroxyl, phosphate or amino protecting group and the method comprises a step of cleaving said at least hydroxyl, phosphate or amino protecting group prior to reacting the oligonucleotide with the amine.

In some embodiments of any one of the aspects described herein, an oligonucleotide described herein comprises from 3 to 50 nucleotides.

In some embodiments of any one of the aspects described herein, an oligonucleotide described herein comprises at least one ribonucleotide (e.g., 2′-OH).

In some embodiments of any one of the aspects described herein, an oligonucleotide described herein at least one 2′-deoxyribonucleotide.

In some embodiments of any one of the aspects described herein, an oligonucleotide described herein comprises at least one nucleotide with a modified or non-natural nucleobase.

In some embodiments of any one of the aspects described herein, an oligonucleotide described herein comprises at least one nucleotide with a modified ribose sugar.

In some embodiments of any one of the aspects described herein, an oligonucleotide described herein oligonucleotide comprises at least one nucleotide comprising a group other than H or OH at the 2′-position of the ribose sugar.

In some embodiments of any one of the aspects described herein, an oligonucleotide described herein comprises at least one nucleotide with a 2′-F ribose.

In some embodiments of any one of the aspects described herein, an oligonucleotide described herein comprises at least one nucleotide with a 2′-OMe ribose.

In some embodiments of any one of the aspects described herein, an oligonucleotide described herein comprises at least one nucleotide comprising a moiety other than a ribose sugar.

In some embodiments of any one of the aspects described herein, an oligonucleotide described herein comprises at least one modified internucleotide linkage.

In some embodiments of any one of the aspects described herein, an oligonucleotide described herein comprises a nucleoside comprising at least one hydroxyl, phosphate or amino protecting group

In some embodiments of any one of the aspects described herein, an oligonucleotide described herein comprises at least one ligand.

Also provided herein are double-stranded nucleic acids, e.g., double-stranded RNA comprising a first strand and a second strand, where at least one of the first or second strand is an oligonucleotide described herein. For example, a double-stranded RNA comprising a first strand and a second strand, where the first and/or the second strand is an oligonucleotide comprising a nucleotide of Formula (II).

In another aspect, provided herein is a method for inhibiting or reducing the expression of a target gene in a subject. The method comprises administering to the subject: (i) a double-stranded RNA described herein, wherein one of the strands of the dsRNA is complementary to a target gene; and/or (ii) an oligonucleotide described herein, wherein the oligonucleotide is complementary to a target gene.

### BRIEF DESCRIPTION OF THE DRAWINGS

FIGS. 1-5 depict compounds according to some exemplary embodiments of the aspects described herein.

FIG. 6 depicts post-synthesis conjugation scheme for preparing an oligonucleotide according to an exemplary embodiment. In FIG. 6, L1 is any conjugate group, such as lipids, GalNac, folate, mannose, RUPA, RGD, peptide.

FIG. 7 shows structures of exemplary monomers C16 (Uhd), Y179, Y180, Y182, Y184, Y209, Y208, and Y210.

FIG. 8 shows dsRNA comprising exemplary monomers of the disclosure have comparable activities than the monomer C16 in brains.

FIGS. 9A and 9B show exemplary dsRNA molecules of the disclosure having strong RNAi activity in the brain also have robust RNAi activity in the heart and liver.

FIG. 10 depicts structures of MOE style C16 monomers.

FIG. 11 shows dsRNA comprising exemplary MOE style C16 monomers (FIG. 10) have similar activity as the control dsRNA.

FIG. 12 depicts structures of exemplary monomers Y250, Y270 and Uhd.

FIG. 13 shows SOD1 knockdown with dsRNAs comprising lipophilic monomer Y250, Y270 or Uhd.

FIG. 14 depicts structures of exemplary monomers Uhd (C16), Y180 (MOE-C16), Y250 (NMA-C16) and Y152 (flipped C16).

FIG. 15 shows mTTR knockdown in mouse eye with dsRNAs comprising the monomer Uhd (C16), Y180 (MOE-C16), Y250 (NMA-C16) or Y152 (flipped C16).

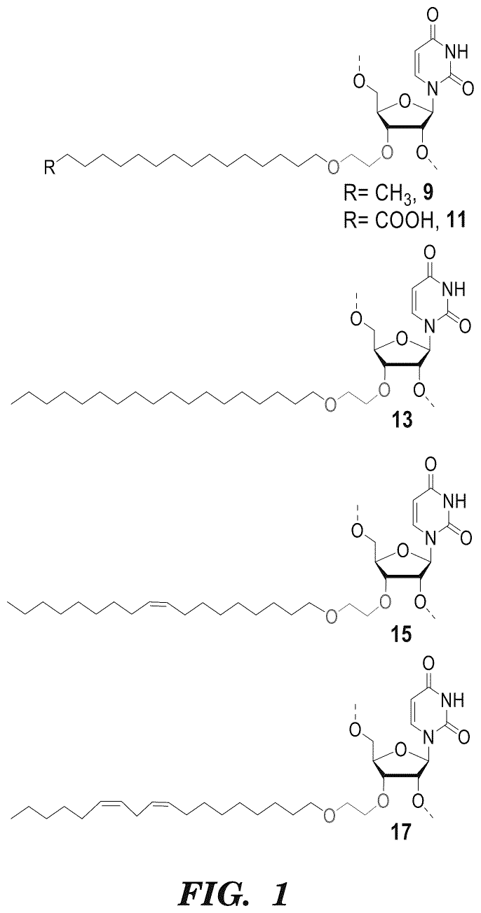

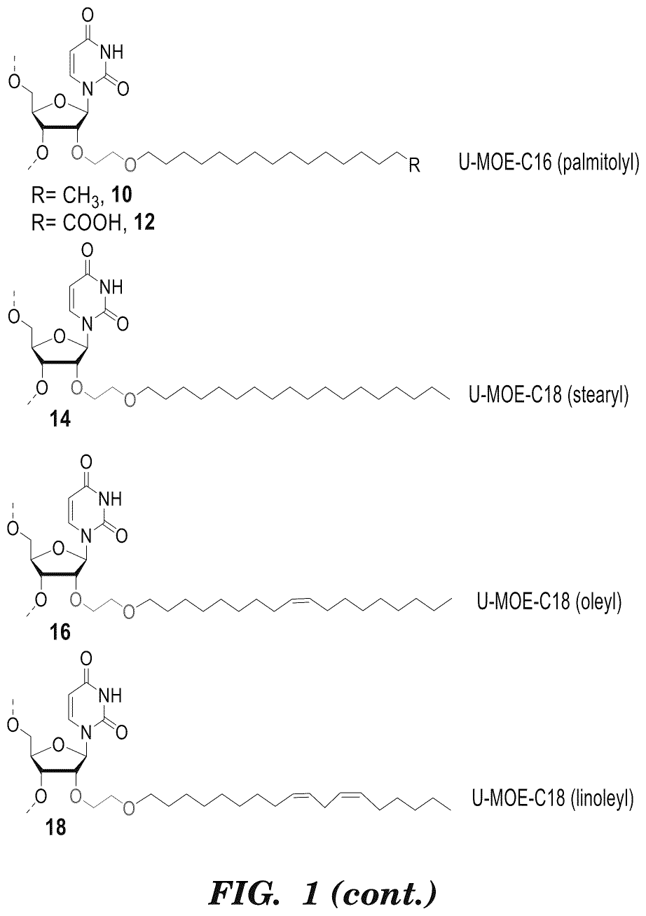

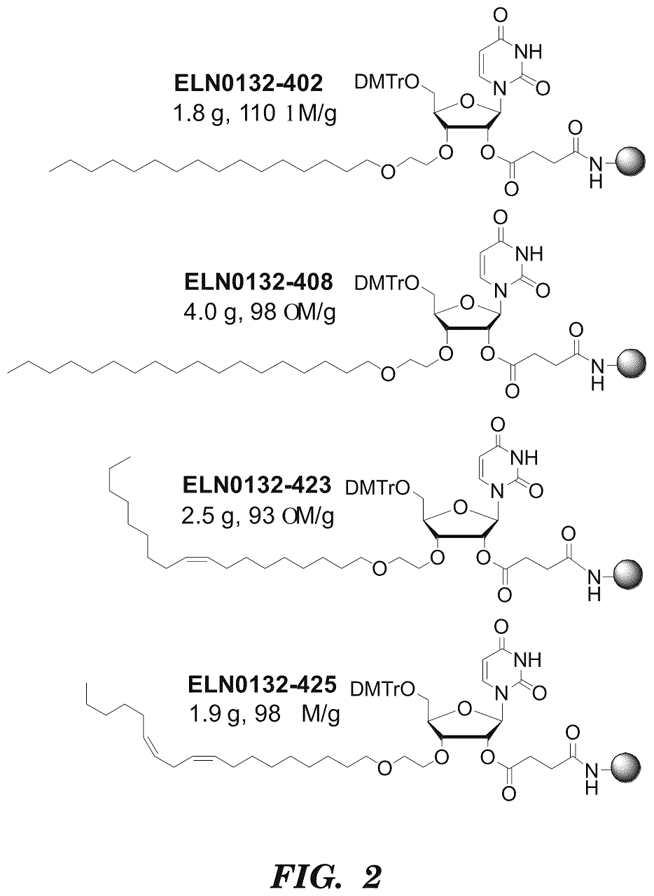

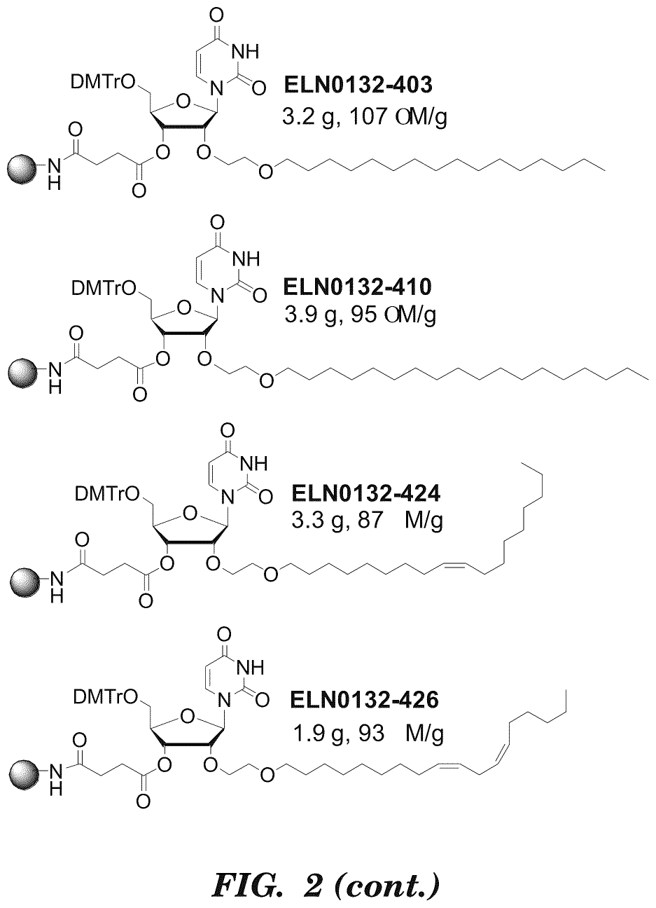

### DETAILED DESCRIPTION

It is to be understood that both the foregoing general description and the following detailed description are exemplary and explanatory only and are not restrictive of the invention, as claimed. Herein, the use of the singular includes the plural unless specifically stated otherwise. As used herein, the use of “or” means “and/or” unless stated otherwise. Furthermore, the use of the term “including” as well as other forms, such as “includes” and “included”, is not limiting. Also, terms such as “element” or “component” encompass both elements and components comprising one unit and elements and components that comprise more than one subunit, unless specifically stated otherwise.

The section headings used herein are for organizational purposes only and are not to be construed as limiting the subject matter described. All documents, or portions of documents, cited in this application, including, but not limited to, patents, patent applications, articles, books, and treatises, are hereby expressly incorporated by reference in their entirety for any purpose.

### X^{M′}

Embodiments of the any one of the aspects described herein include X^{M′}. In some embodiments of the any one of the aspects described herein, X^{M′} is O. In some other embodiments of the any one of the aspects described herein, X^{M′} is S. In yet some other embodiment of the any one of the aspects described herein, X^{M′} is N(R^{MX}), wherein R^{MX} is hydrogen or R^{M′}.

### Y^{M}

Embodiments of the any one of the aspects described herein include Y^{M}. In some embodiments of the any one of the aspects described herein, Y^{M′} is O. In some other embodiments of the any one of the aspects described herein, Y^{M} is S.

### R^{M′}

Embodiments of the any one of the aspects described herein include R^{M′}. In some embodiments, R^{M′} is optionally substituted C₆₋₃₀ alkyl, optionally substituted C₆₋₃₀alkenyl, optionally substituted C₆₋₃₀alkynyl, or optionally substituted C₃₋₃₀cycloalkyl, optionally substituted 3-8 membered heterocyclylC₃₋₃₀alkyl, optionally substituted C₃₋₁₀cycloalkylC₃₋₃₀alkyl; optionally substituted arylC₃₋₃₀alkyl, optionally substituted heteroarylC₃₋₃₀alkyl, optionally substituted C₁₋₃₀ alkoxyC₁₋₃₀alkyl, —(CH₂CH₂O)_{mq}—R^{MQ}, a lipid, a ligand, a linker, or a linker to one or more ligands, wherein mq is an integer selected from 1-10 and R^{MQ} is hydrogen or C₁₋₆alkyl.

In some embodiments, R^{M′} is C₆₋₃₀ alkyl, C₆₋₃₀alkenyl or C₆₋₃₀alkynyl, where the C₆₋₃₀ alkyl, C₆₋₃₀alkenyl and C₆₋₃₀alkynyl is optionally substituted with at least one substituent. For example, R^{M′} is C₆₋₃₀alkyl, C₆₋₃₀alkenyl or C₆₋₃₀alkynyl, where the C₆₋₃₀alkyl, C₆₋₃₀alkenyl and C₆₋₃₀alkynyl is optionally substituted with at least one substituent selected from the group consisting of halogen, hydroxy, caboxy, oxo, nitro, haloalkyl, alkyl, alkenyl, alkynyl, alkaryl, aryl, heteroaryl, cyclyl, heterocyclyl, aralkyl, alkoxy, aryloxy, amino, alkylamino, dialkylamino, acylamino, alkylcarbanoyl, arylcarbanoyl, aminoalkyl, alkoxycarbonyl, carboxy, hydroxyalkyl, alkanesulfonyl, arenesulfonyl, alkanesulfonamido, arenesulfonamido, aralkylsulfonamido, alkylcarbonyl, acyloxy, cyano or ureido. For example, the C₆₋₃₀alkyl, C₆₋₃₀alkenyl and C₆₋₃₀alkynyl is optionally substituted with 1, 2, 3, 4 or 5 groups selected from OH, CN, —SC(O)Ph, oxo (═O), SH, SO₂NH₂, SO₂(C₁-C₄)alkyl, SO₂NH(C₁-C₄)alkyl, halogen, carbonyl, thiol, cyano, NH₂, NH(C₁-C₄)alkyl, N[(C₁-C₄)alkyl]₂, C(O)NH₂, COOH, COOMe, acetyl, (C₁-C₈)alkyl, O(C₁-C₈)alkyl, O(C₁-C₈)haloalkyl, (C₂-C₈)alkenyl, (C₂-C₈)alkynyl, haloalkyl, thioalkyl, cyanomethylene, alkylaminyl, aryl, heteroaryl, substituted aryl, NH₂—C(O)-alkylene, NH(Me)-C(O)-alkylene, CH₂—C(O)-alkyl, C(O)-alkyl, alkylcarbonylaminyl, CH₂—[CH(OH)]ₘ—(CH₂)ₚ—OH, CH₂—[CH(OH)]ₘ—(CH₂)ₚ—NH₂ or CH₂-aryl-alkoxy; “in” and “p” are independently 1, 2, 3, 4, 5 or 6.

In some embodiments, R^{M′} is C₆₋₃₀ alkyl, C₆₋₃₀alkenyl or C₆₋₃₀alkynyl, where the C₆₋₃₀ alkyl, C₆₋₃₀alkenyl and C₆₋₃₀alkynyl is optionally substituted at an end with at least one substituent. “alyl

In some embodiments, R^{M′} is C₆₋₃₀ alkyl, C₆₋₃₀alkenyl or C₆₋₃₀alkynyl, where the C₆₋₃₀ alkyl, C₆₋₃₀alkenyl and C₆₋₃₀alkynyl is optionally substituted at an end with at least one substituent selected from the group consisting of halogen, hydroxy, caboxy, oxo, nitro, haloalkyl, alkyl, alkenyl, alkynyl, alkaryl, aryl, heteroaryl, cyclyl, heterocyclyl, aralkyl, alkoxy, aryloxy, amino, alkylamino, dialkylamino, acylamino, alkylcarbanoyl, arylcarbanoyl, aminoalkyl, alkoxycarbonyl, carboxy, hydroxyalkyl, alkanesulfonyl, arenesulfonyl, alkanesulfonamido, arenesulfonamido, aralkylsulfonamido, alkylcarbonyl, acyloxy, cyano or ureido. For example, R^{M′} is R^{M′} is C₆₋₃₀ alkyl, C₆₋₃₀alkenyl or C₆₋₃₀alkynyl, where the C₆₋₃₀ alkyl, C₆₋₃₀alkenyl and C₆₋₃₀alkynyl is optionally substituted the end away from the point where R^{M′} is attached to rest of the molecule with at least one substituent selected from the group consisting of OH, CN, —SC(O)Ph, oxo (═O), SH, SO₂NH₂, SO₂(C₁-C₄)alkyl, SO₂NH(C₁-C₄)alkyl, halogen, carbonyl, thiol, cyano, NH₂, NH(C₁-C₄)alkyl, N[(C₁-C₄)alkyl]₂, C(O)NH₂, COOH, COOMe, acetyl, (C₁-C₈)alkyl, O(C₁-C₈)alkyl, O(C₁-C₈)haloalkyl, (C₂-C₈)alkenyl, (C₂-C₈)alkynyl, haloalkyl, thioalkyl, cyanomethylene, alkylaminyl, aryl, heteroaryl, substituted aryl, NH₂—C(O)-alkylene, NH(Me)-C(O)-alkylene, CH₂—C(O)-alkyl, C(O)-alkyl, alkylcarbonylaminyl, CH₂—[CH(OH)]ₘ—(H₂)ₚ—OH, CH₂—[CH(OH)]ₘ—(CH₂)ₚ—NH₂ or CH₂-aryl-alkoxy; “m” and “p” are independently 1, 2, 3, 4, 5 or 6.

In some embodiments of any one of the aspects described herein, R^{M′} is C₃₋₃₀ alkyl, C₃₋₃₀alkenyl or C₃₋₃₀alkynyl, where the C₃₋₃₀ alkyl, C₃₋₃₀alkenyl and C₃₋₃₀alkynyl is substituted at an end with an optionally substituted 3-8 membered heterocyclyl, optionally substituted C₃₋₁₀cycloalkyl, optionally substituted aryl or optionally substituted heteroaryl, For example, R^{M′} is C₃₋₃₀ alkyl, C₃₋₃₀alkenyl or C₃₋₃₀alkynyl, where the C₃₋₃₀ alkyl, C₃₋₃₀alkenyl and C₃₋₃₀alkynyl is substituted with an optionally substituted 3-8 membered heterocyclyl, optionally substituted C₃₋₁₀cycloalkyl, optionally substituted aryl or optionally substituted heteroaryl at the end away from the point where R^{M′} is attached to rest of the molecule. It is noted that the C₃₋₃₀ alkyl, C₃₋₃₀alkenyl and C₃₋₃₀alkynyl may optionally be substituted with 1, 2, 3, 4 or more additional independently selected substituents.

In some embodiments of any one of the aspects described herein, R^{M′} is —(CH₂)_{mc}—R^{MC}, where mc is an integer from 3 to 30, and R^{MC} is an optionally substituted 3-8 membered heterocyclyl, optionally substituted C₃₋₁₀cycloalkyl, optionally substituted aryl or optionally substituted heteroaryl.

In some embodiments of any one of the aspects described herein, R^{M′} is a C₁₋₃₀ alkyl, C₂₋₃₀alkenyl or C₂₋₃₀alkynyl, where the C₁₋₃₀ alkyl, C₂₋₃₀alkenyl and C₂₋₃₀alkynyl substituted at an end with an optionally substituted C₁₋₃₀ alkoxy. For example, R^{M′} is C₁₋₃₀ alkyl, C₂₋₃₀alkenyl or C₂₋₃₀alkynyl, where the C₁₋₃₀ alkyl, C₂₋₃₀alkenyl and C₂₋₃₀alkynyl is substituted with an optionally substituted C₁₋₃₀ alkoxy at the end away from the point where R^{M′} is attached to rest of the molecule. It is noted that the C₁₋₃₀ alkyl, C₂₋₃₀alkenyl and C₂₋₃₀alkynyl may optionally be substituted with 1, 2, 3, 4 or more additional independently selected substituents.

In some embodiments of any one of the aspects described herein, R^{M′} is —(CH₂)_{md}—R^{MD}, where md is an integer from 1 to 30, and R^{MD} is an optionally substituted an optionally substituted C₁₋₃₀ alkoxy.

In some embodiments of any one of the aspects described herein, R^{M″} is —(CH₂CH₂O)_{mq}—R^{MQ}, wherein mq is an integer selected from 1-10 and R^{MQ} I at the end away from the point where R^{M′} is attached to rest of the molecule s hydrogen or C₁₋₆alkyl. For example, R^{M″} is —(CH₂CH₂O)_{mq}—R^{MQ}, where mq is 1, 2, 3, 4, 5, 6, 7, 8, 9 or 10, and R^{MQ} is H. In another non-limiting example, R^{M″} is —(CH₂CH₂O)_{mq}—R^{MQ}, where mq is 1, 2, 3, 4, 5, 6, 7, 8, 9 or 10, and R^{MQ} is C₁₋₆alkyl, such as methyl, ethyl, propyl, i-propyl, n-butyl, t-butyl, n-pentyl, or hexyl, It is noted that C₁₋₆alkyl group of R^{MQ} can be optionally substituted with 1, 2, 3, 4 or more independently selected substituents In some embodiments of any one of the aspects described herein, R^{M′} is C₆₋₃₀ alkyl, C₆₋₃₀alkenyl or C₆₋₃₀alkynyl, where the C₆₋₃₀ alkyl, C₆₋₃₀alkenyl and C₆₋₃₀alkynyl is substituted with an anionic group or a cationic group at the end away from the point where R^{M′} is attached to rest of the molecule.

In some embodiments of any one of the aspects described herein, R^{M′} is —(CH₂)ₘₘ—R^{ME}, where mm is an integer from 6 to 29, and R^{ME} is methyl, CO₂H, CO₂Me, NH₂, SH, OH, CH═CH₂ or C≡CH.

In some embodiments of any one of the aspects, R^{M′} is —(CH₂)ₘₘ—R^{ME}, where mm is 10, 11, 12, 13, 14, 15, 16, 17, 18, 19 or 20. For example, mm is 13, 14, 15, 16, 17, 18, 19 or 20. In another non-limiting example, mm is 13, 15, 17 or 19. In yet another non-limiting example, mm is 14, 16, 18 or 20.

In some embodiments, of any one of the aspects, R^{ME} is methyl, CO₂H, CO₂Me or NH₂. For example, R^{ME} is CO₂H, CO₂Me or NH₂.

In some embodiments of any one of the aspects, mm is 10, 11, 12, 13, 14, 15, 16, 17, 18, 19 or 20, and R^{ME} is methyl, CO₂H, CO₂Me or NH₂. For example, mm is 14, 16, 18 or 20, and R^{ME} is CO₂H, CO₂Me or NH₂. In another non-limiting example, mm is 13, 15, 17 or 19, and R^{ME} is methyl.

In some embodiments of any one of the aspects described herein, R^{M′} is hexyl, heptyl, octyl, nonyl, decyl, undecyl, dodecyl, tridecyl, tetradecyl, pentadecyl, hexadecyl, heptadecyl, octadecyl, nonadecyl, or icosadecyl. For example, R^{M′} is tetradecyl, pentadecyl, hexadecyl, heptadecyl, octadecyl, nonadecyl, or icosadecyl. In some embodiments, R^{M′} is tetradecyl, hexadecyl, octadecyl, or icosadecyl.

In some embodiments of any one of the aspects described herein, R^{M′} is hexyl, heptyl, octyl, nonyl, decyl, undecyl, dodecyl, tridecyl, tetradecyl, pentadecyl, hexadecyl, heptadecyl, octadecyl, nonadecyl, or icosadecyl, each of which is substituted with at least one substituent selected from the group consisting of halogen, hydroxy, caboxy, oxo, nitro, haloalkyl, alkyl, alkenyl, alkynyl, alkaryl, aryl, heteroaryl, cyclyl, heterocyclyl, aralkyl, alkoxy, aryloxy, amino, alkylamino, dialkylamino, acylamino, alkylcarbanoyl, arylcarbanoyl, aminoalkyl, alkoxycarbonyl, carboxy, hydroxyalkyl, alkanesulfonyl, arenesulfonyl, alkanesulfonamido, arenesulfonamido, aralkylsulfonamido, alkylcarbonyl, acyloxy, cyano or ureido. For example, R^{M′} is hexyl, heptyl, octyl, nonyl, decyl, undecyl, dodecyl, tridecyl, tetradecyl, pentadecyl, hexadecyl, heptadecyl, octadecyl, nonadecyl, or icosadecyl, each of which is substituted with at least one substituent selected from the group consisting of OH, CN, —SC(O)Ph, oxo (═O), SH, SO₂NH₂, SO₂(C₁-C₄)alkyl, SO₂NH(C₁-C₄)alkyl, halogen, carbonyl, thiol, cyano, NH₂, NH(C₁-C₄)alkyl, N[(C₁-C₄)alkyl]₂, C(O)NH₂, COOH, COOMe, acetyl, (C₁-C₈)alkyl, O(C₁-C₈)alkyl, O(C₁-C₈)haloalkyl, (C₂-C₈)alkenyl, (C₂-C₈)alkynyl, haloalkyl, thioalkyl, cyanomethylene, alkylaminyl, aryl, heteroaryl, substituted aryl, NH₂—C(O)-alkylene, NH(Me)-C(O)-alkylene, CH₂—C(O)-alkyl, C(O)-alkyl, alkylcarbonylaminyl, CH₂—[CH(OH)]ₘ—(CH₂)ₚ—OH, CH₂—[CH(OH)]ₘ—(CH₂)ₚ—NH₂ or CH₂-aryl-alkoxy; “m” and “p” are independently 1, 2, 3, 4, 5 or 6.

In some embodiments, R^{M′} is hexyl, heptyl, octyl, nonyl, decyl, undecyl, dodecyl, tridecyl, tetradecyl, pentadecyl, hexadecyl, heptadecyl, octadecyl, nonadecyl, or icosadecyl, each of which is substituted at the end away from the point where R^{M′} is attached to rest of the molecule with at least one substituent selected from the group consisting of OH, CN, —SC(O)Ph, oxo (═O), SH, SO₂NH₂, SO₂(C₁-C₄)alkyl, SO₂NH(C₁-C₄)alkyl, halogen, carbonyl, thiol, cyano, NH₂, NH(C₁-C₄)alkyl, N[(C₁-C₄)alkyl]₂, C(O)NH₂, COOH, COOMe, acetyl, (C₁-C₈)alkyl, O(C₁-C₈)alkyl, O(C₁-C₈)haloalkyl, (C₂-C₈)alkenyl, (C₂-C₈)alkynyl, haloalkyl, thioalkyl, cyanomethylene, alkylaminyl, aryl, heteroaryl, substituted aryl, NH₂—C(O)-alkylene, NH(Me)-C(O)-alkylene, CH₂—C(O)-alkyl, C(O)-alkyl, alkylcarbonylaminyl, CH₂—[CH(OH)]ₘ—(CH₂)ₚ—OH, CH₂—[CH(OH)]ₘ—(CH₂)ₚ—NH₂ or CH₂-aryl-alkoxy; “m” and “p” are independently 1, 2, 3, 4, 5 or 6. For example, R^{ME} is dodecyl, tridecyl, tetradecyl, pentadecyl, hexadecyl, heptadecyl, octadecyl, nonadecyl, or icosadecyl, each of which is substituted at the end away from the point where R^{M′} is attached to rest of the molecule with OH, oxo (═O), SH, NH₂, NH(C₁-C₄)alkyl, N[(C₁-C₄)alkyl]₂, C(O)NH₂, COOH, or COOMe.

In some embodiments, R^{M′} is tetradecyl, pentadecyl, hexadecyl, heptadecyl, octadecyl, nonadecyl, or icosadecyl, each of which is substituted at the end away from the point where R^{M′} is attached to rest of the molecule with NH₂, CO₂H, or CO₂Me.

In some embodiments of any one of the aspects described herein, R^{M′} is (9Z)-tetradec-9-enyl, (6Z)-Hexadec-6-enyl, (9Z)-hexadec-9-enyl, (9Z)-octadec-9-enyl, (9E)-octadec-9-enyl, (11E)-octadec-11-enyl, (9Z,12Z)-octadeca-9,12-dienyl, (9E,12E)-octadeca-9,12-dienyl, (9Z,12Z,15Z)-octadeca-9,12,15-trienyl, (5Z,8Z,11Z,14Z)-icosa-5,8,11,14-tetraenyl, (5Z,8Z,11Z,14Z,17Z)-Icosa-5,8,11,14,17-pentaenyl, or (13Z)-docos-13-enyl. For example, R^{M′} is (9Z)-octadec-9-enyl, (9E)-octadec-9-enyl, (11E)-octadec-11-enyl, (9Z,12Z)-octadeca-9,12-dienyl or (9E,12E)-octadeca-9,12-dienyl. In some embodiments, R^{M′} is (9Z)-octadec-9-enyl or (9Z,12Z)-octadeca-9,12-dienyl or (9E,12E)-octadeca-9,12-dienyl.

In some embodiments of any one of the aspects described herein, when R² is —OCH₂CH₂—O—R^{M′}; R⁵ is hydroxyl or protected hydroxyl; R⁴ is H; and R³ is hydroxyl, protected hydroxyl, a phosphate group or a reactive phosphorous group, then R^{M′} is not unsubstituted C₆₋₂₁ alkyl, unsubstituted C₆₋₂₁alkenyl, or unsubstituted C₆₋₂₁alkynyl.

In some embodiments of any one of the aspects described herein, R^{M′} is a ligand, a linker, or a linker to one or more ligands. For example, R^{M′} is -L-R^{L}, where L is a linker and R^{L} is a ligand.

R^{N′} and R^{N″}

Embodiments of the any one of the aspects described herein include R^{N′} and R^{N″}. In embodiments of the any one of the aspects described herein, R^{N′} and R^{N″} independently are hydrogen, optionally substituted C₆₋₃₀ alkyl, optionally substituted C₆₋₃₀alkenyl, optionally substituted C₆₋₃₀alkynyl, or optionally substituted C₃₋₃₀cycloalkyl; a lipid, a ligand, a linker, or a linker to one or more ligands. Optionally, at least one of R^{N′} and R^{N″} is not hydrogen.

In some embodiments of any one of the aspects described herein, one of R^{N′} and R^{N″} is hydrogen.

In some embodiments of any one of the aspects described herein, neither one of R^{N′} and R^{N″} is hydrogen. It is noted that when neither one of R^{N′} and R^{N″} is hydrogen, then R^{N′} and R^{N″} can be the same or different. Accordingly, in some embodiments of any one of the aspects described herein, neither one of R^{N′} and R^{N″} is hydrogen, and R^{N′} and R^{N″} are the same. In some other embodiments of any one of the aspects described herein, neither one of R^{N′} and R^{N″} is hydrogen, and R^{N′} and R^{N″} are different.

In some embodiments, at least one of R^{N′} and R^{N″} is C₆₋₃₀ alkyl, C₆₋₃₀alkenyl or C₆₋₃₀alkynyl, where the C₆₋₃₀ alkyl, C₆₋₃₀alkenyl and C₆₋₃₀alkynyl is optionally substituted with at least one substituent. For example, at least one of R^{N′} and R^{N″} is C₆₋₃₀alkyl, C₆₋₃₀alkenyl or C₆₋₃₀alkynyl, where the C₆₋₃₀alkyl, C₆₋₃₀alkenyl and C₆₋₃₀alkynyl is optionally substituted with at least one substituent selected from the group consisting of halogen, hydroxy, caboxy, oxo, nitro, haloalkyl, alkyl, alkenyl, alkynyl, alkaryl, aryl, heteroaryl, cyclyl, heterocyclyl, aralkyl, alkoxy, aryloxy, amino, alkylamino, dialkylamino, acylamino, alkylcarbanoyl, arylcarbanoyl, aminoalkyl, alkoxycarbonyl, carboxy, hydroxyalkyl, alkanesulfonyl, arenesulfonyl, alkanesulfonamido, arenesulfonamido, aralkylsulfonamido, alkylcarbonyl, acyloxy, cyano or ureido. For example, the C₆₋₃₀alkyl, C₆₋₃₀alkenyl and C₆₋₃₀alkynyl is optionally substituted with 1, 2, 3, 4 or 5 groups selected from OH, CN, —SC(O)Ph, oxo (═O), SH, SO₂NH₂, SO₂(C₁-C₄)alkyl, SO₂NH(C₁-C₄)alkyl, halogen, carbonyl, thiol, cyano, NH₂, NH(C₁-C₄)alkyl, N[(C₁-C₄)alkyl]₂, C(O)NH₂, COOH, COOMe, acetyl, (C₁-C₈)alkyl, O(C₁-C₈)alkyl, O(C₁-C₈)haloalkyl, (C₂-C₈)alkenyl, (C₂-C₈)alkynyl, haloalkyl, thioalkyl, cyanomethylene, alkylaminyl, aryl, heteroaryl, substituted aryl, NH₂—C(O)-alkylene, NH(Me)-C(O)-alkylene, CH₂—C(O)-alkyl, C(O)-alkyl, alkylcarbonylaminyl, CH₂—[CH(OH)]ₘ—(CH₂)ₚ—OH, CH₂—[CH(OH)]ₘ—(CH₂)ₚ—NH₂ or CH₂-aryl-alkoxy; “in” and “p” are independently 1, 2, 3, 4, 5 or 6.

In some embodiments, at least one of R^{N′} and R^{N″} is C₆₋₃₀ alkyl, C₆₋₃₀alkenyl or C₆₋₃₀alkynyl, where the C₆₋₃₀ alkyl, C₆₋₃₀alkenyl and C₆₋₃₀alkynyl is optionally substituted at an end with at least one substituent. For example, at least one of R^{N′} and R^{N″} is C₆₋₃₀ alkyl, C₆₋₃₀alkenyl or C₆₋₃₀alkynyl, where the C₆₋₃₀ alkyl, C₆₋₃₀alkenyl and C₆₋₃₀alkynyl is optionally substituted at least one substituent at the end away from the point where at least one of R^{N′} and R^{N″} is attached to rest of the molecule.

In some embodiments, at least one of R^{N′} and R^{N″} is C₆₋₃₀ alkyl, C₆₋₃₀alkenyl or C₆₋₃₀alkynyl, where the C₆₋₃₀ alkyl, C₆₋₃₀alkenyl and C₆₋₃₀alkynyl is optionally substituted at an end with at least one substituent selected from the group consisting of halogen, hydroxy, caboxy, oxo, nitro, haloalkyl, alkyl, alkenyl, alkynyl, alkaryl, aryl, heteroaryl, cyclyl, heterocyclyl, aralkyl, alkoxy, aryloxy, amino, alkylamino, dialkylamino, acylamino, alkylcarbanoyl, arylcarbanoyl, aminoalkyl, alkoxycarbonyl, carboxy, hydroxyalkyl, alkanesulfonyl, arenesulfonyl, alkanesulfonamido, arenesulfonamido, aralkylsulfonamido, alkylcarbonyl, acyloxy, cyano or ureido. For example, at least one of R^{N′} and R^{N″} is at least one of R^{N′} and R^{N″} is C₆₋₃₀ alkyl, C₆₋₃₀alkenyl or C₆₋₃₀alkynyl, where the C₆₋₃₀ alkyl, C₆₋₃₀alkenyl and C₆₋₃₀alkynyl is optionally substituted the end away from the point where at least one of R^{N′} and R^{N″} is attached to rest of the molecule with at least one substituent selected from the group consisting of OH, CN, —SC(O)Ph, oxo (═O), SH, SO₂NH₂, SO₂(C₁-C₄)alkyl, SO₂NH(C₁-C₄)alkyl, halogen, carbonyl, thiol, cyano, NH₂, NH(C₁-C₄)alkyl, N[(C₁-C₄)alkyl]₂, C(O)NH₂, COOH, COOMe, acetyl, (C₁-C₈)alkyl, O(C₁-C₈)alkyl, O(C₁-C₈)haloalkyl, (C₂-C₈)alkenyl, (C₂-C₈)alkynyl, haloalkyl, thioalkyl, cyanomethylene, alkylaminyl, aryl, heteroaryl, substituted aryl, NH₂—C(O)-alkylene, NH(Me)-C(O)-alkylene, CH₂—C(O)-alkyl, C(O)-alkyl, alkylcarbonylaminyl, CH₂—[CH(OH)]ₘ—(CH₂)ₚ—OH, CH₂—[CH(OH)]ₘ—(CH₂)ₚ—NH₂ or CH₂-aryl-alkoxy; “in” and “p” are independently 1, 2, 3, 4, 5 or 6.

In some embodiments of any one of the aspects described herein, at least one of R^{N′} and R^{N″} is —(CH₂)ₘₙ—R^{NE}, where mn is an integer from 6 to 29, and R^{NE} is methyl, CO₂H, CO₂Me, NH₂, SH, OH, CH═CH₂ or C≡CH.

In some embodiments of any one of the aspects, at least one of R^{N′} and R^{N″} is —(CH₂)ₘₙ—R^{NE}, where mn is 10, 11, 12, 13, 14, 15, 16, 17, 18, 19 or 20. For example, mn is 10, 11, 12, 13, 14, 15, 16, or 17. In another non-limiting example, mn is 11, 13, 15 or 17. In yet another non-limiting example, mn is 10, 12, 14, 16 or 18.

In some embodiments, of any one of the aspects, R^{NE} is methyl, CO₂H, CO₂Me, NH₂, CH═CH₂ or C≡CH. For example, R^{NE} CO₂H, CO₂Me, NH₂, CH═CH₂ or C≡CH.

In some embodiments of any one of the aspects, mn is 9, 10, 11, 12, 13, 14, 15, 16, 17, 18, 19 or 20, and R^{NE} is methyl, CO₂H, CO₂Me, NH₂, CH═CH₂ or C≡CH. For example, mn is 9, 11, 13, 15 or 17, and R^{NE} is CO₂H, CO₂Me, NH₂, CH═CH₂ or C≡CH. In another non-limiting example, mn is 10, 12, 14, 16 or 18, and R^{NE} is CO₂H, CO₂Me, NH₂, CH═CH₂ or C≡CH. In yet another non-limiting example, mn is 9, 11, 13, 15, 17 or 19, and R^{NE} is methyl. In still another non-limiting example, mn is 10, 12, 14, 16 or 18, and R^{NE} is methyl.

In some embodiments of any one of the aspect, R^{N′} and R^{N″} are selected independently from C₆₋₁₀alkyl, C₆₋₁₀alkenyl or C₆₋₁₀alkynyl, where the C₆₋₁₀alkyl, C₆₋₁₀alkenyl and C₆₋₁₀alkynyl is optionally substituted with at least one substituent.

In some embodiments of any one of the aspects described herein, at least one of R^{N′} and R^{N″} is hexyl, heptyl, octyl, nonyl, decyl, undecyl, dodecyl, tridecyl, tetradecyl, pentadecyl, hexadecyl, heptadecyl, octadecyl, nonadecyl, or icosadecyl.

In some embodiments of any one of the aspects described herein, at least one of R^{N′} and R^{N″} is (9Z)-tetradec-9-enyl, (6Z)-Hexadec-6-enyl, (9Z)-hexadec-9-enyl, (9Z)-octadec-9-enyl, (9E)-octadec-9-enyl, (11E)-octadec-11-enyl, (9Z,12Z)-octadeca-9,12-dienyl, (9E,12E)-octadeca-9,12-dienyl, (9Z,12Z,15Z)-octadeca-9,12,15-trienyl, (5Z,8Z,11Z,14Z)-icosa-5,8,11,14-tetraenyl, (5Z,8Z,11Z,14Z,17Z)-Icosa-5,8,11,14,17-pentaenyl, or (13Z)-docos-13-enyl. For example, at least one of R^{N′} and R^{N″} is (9Z)-octadec-9-enyl, (9E)-octadec-9-enyl, (11E)-octadec-11-enyl, (9Z,12Z)-octadeca-9,12-dienyl or (9E,12E)-octadeca-9,12-dienyl. In some embodiments, at least one of R^{N′} and R^{N″} is (9Z)-octadec-9-enyl or (9Z,12Z)-octadeca-9,12-dienyl or (9E,12E)-octadeca-9,12-dienyl.

In some embodiments of any one of the aspects described herein, at least one of R^{N′} and R^{N″} is an optionally substituted C₃₋₃₀cycloalkyl. It is noted that the cycloalkyl can be partially unsaturated, i.e., the cyclcoalkyl can comprise one or more double and/or triple bonds. For example, at least one of R^{N′} and R^{N″} is cyclopentyl, cyclohexyl, cycloheptyl, cyclooctyl, cyclononyl, cyclodecyl, cycloundecyl or cyclododecyl. In some embodiments, at least one of R^{N′} and R^{N″} is cyclohexyl, cycloheptyl, cyclooctyl, cyclononyl, cyclodecyl. For example, at least one of R^{N′} and R^{N″} is cyclooctyl.

In some embodiments of any one of the aspects described herein, at least one of R^{N′} and R^{N″} is a ligand, a linker, or a linker to one or more ligands. For example, at least one of R^{N′} and R^{N″} is -L-R^{L}, where L is a linker and R^{L} is a ligand. In some embodiments, one of R^{N′} and R^{N″} is hydrogen and the other of R^{N′} and R^{N″} is -L-R^{L}. Exemplary ligands for R^{N′} and R^{N″} include, but are not limited to triGalNAc, monoGalNac, cyclic-RGD and other peptide-targeting ligand, folate, DUPA, biotin, carboxyfluorescein, lipoic acid and mannose ligand.

In some embodiments, at least one of R^{N′} and R^{N″} is

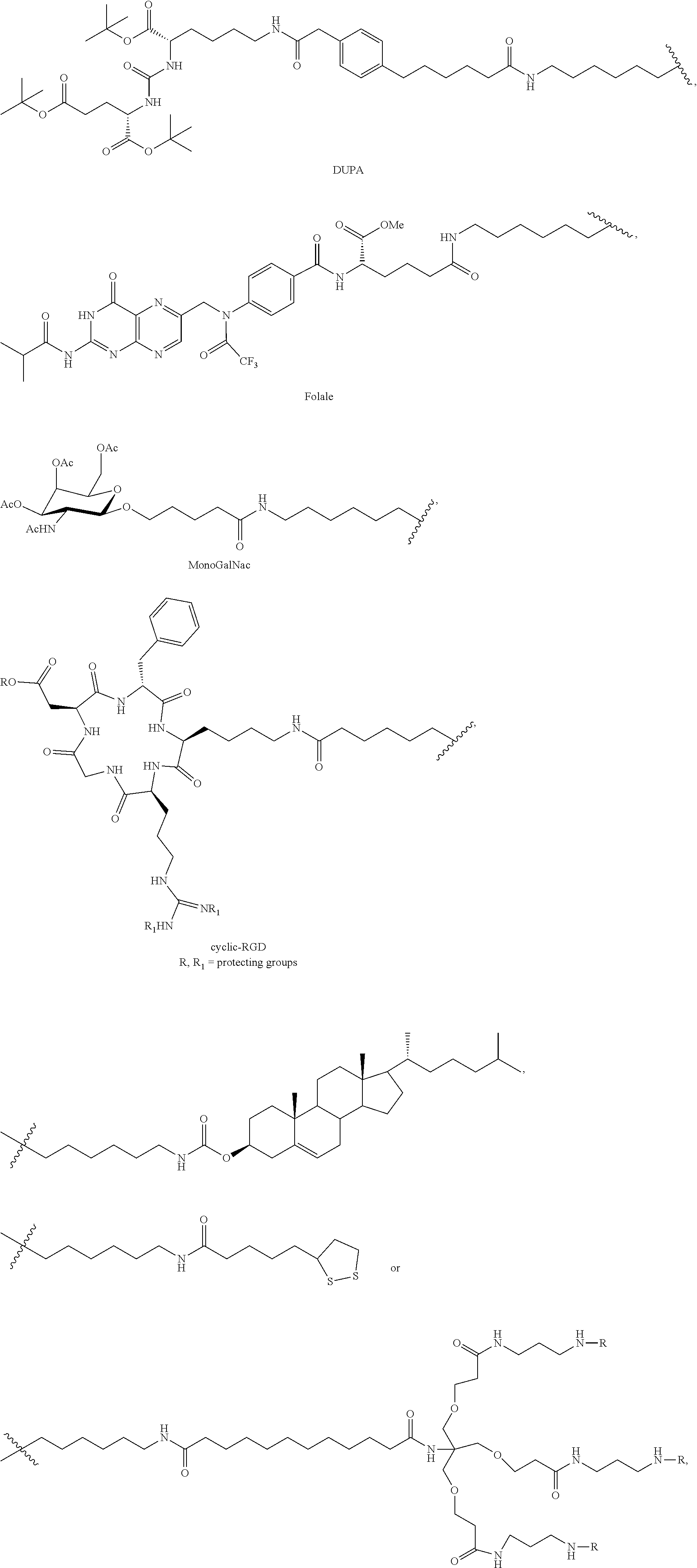

wherein R is

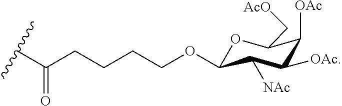

For example, one of R^{N′} and R^{N″} is hydrogen and the other is the structure in the above paragraph.

### R^{LV}

Embodiments of the any one of the aspects described herein include R^{LV}. In some embodiments, R^{LV} is a C₁₋₆alkyl. For example, R^{LV} is methyl, ethyl, propyl, butyl, isobutyl, pentyl, or hexyl. In some embodiments of any one of the aspects described herein, R^{LV} is ethyl.

### R²

In some embodiments of any one of the aspects described herein, R² is —O(CH₂)ₘ₁—X′—R^{M′}, —O(CH₂)ₙ₁—C(O)N(R^{N′})(R^{N″}), hydrogen, hydroxyl, protected hydroxyl, phosphate group, reactive phosphorous group, halogen, optionally substituted C₁₋₃₀ alkyl, optionally substituted C₂₋₃₀alkenyl, optionally substituted C₂₋₃₀alkynyl, optionally substituted C₁₋₃₀ alkoxy (e.g., methoxy, 2-methoxyethoxy), alkoxyalkyl (e.g., methoxyethyl such as 2-methoxyethyl), alkoxyalkylamine, alkoxyoxycarboxylate, amino, alkylamino, dialkylamino, protected aminoalkyl, —O—N-methylacetamido, —O—C₄₋₃₀alkyl-ON(CH₂R⁸)(CH₂R⁹), or —O—C₄₋₃₀alkyl-ON(CH₂R⁸)(CH₂R⁹), a solid support, a linker or a linker covalently attached to a solid support. For example, R² is —O(CH₂)ₘ₁—X′—R^{M′}, —O(CH₂)ₙ₁—C(O)N(R^{N′})(R^{N″}), hydrogen, hydroxyl, protected hydroxyl, phosphate group, reactive phosphorous group, halogen, optionally substituted C₁₋₃₀ alkoxy, alkoxyalkyl (e.g., methoxyethyl), alkoxyalkylamine, alkoxyoxycarboxylate, amino, alkylamino, dialkylamino, —O—N-methylacetamido, or C₆₋₂₄ alkyl (e.g., n-C₆₋₂₄ alkyl).

In some embodiments of the any one of the aspects described herein, R² is —O(CH₂)ₘ₁—X^{M′}—R^{M′}. For example, R² is —O(CH₂)ₘ₁—O—R^{M′}. In another non-limiting example, R² is —O(CH₂)ₘ₁—S—R^{M′}.

In some embodiments of any one of the aspects, R² is —OCH₂CH₂—X′—R^{M′}. For example, R² is —OCH₂CH₂—O—R^{M′}. In another non-limiting example, R² is —OCH₂CH₂—S—R^{M′}.

In some embodiments of any one the aspects described herein, R² is —O(CH₂)ₙ₁—C(O)N(R^{N′})(R^{N″}). It is noted, when R² is —O(CH₂)ₙ₁—C(O)N(R^{N′})(R^{N″}) then n1 can be 1 or 2. Accordingly, in some embodiments of any one the aspects described herein, R² is —OCH₂—C(O)N(R^{N′})(R^{N″}). In some other embodiments of any one of the aspects described herein, R² is —OCH₂CH₂—C(O)N(R^{N′})(R^{N″}).

In some embodiments of any one of the aspects, R² is hydrogen, hydroxyl, halogen, protected hydroxyl, optionally substituted C₁₋₃₀ alkyl, optionally substituted C₂₋₃₀ alkenyl, optionally substituted C₂₋₃₀alkynyl, optionally substituted C₁₋₃₀alkoxy (e.g., methoxy, 2-methoxyethoxy), alkoxyalkyl (e.g., methoxyethyl such a 2-methoxyethyl), alkoxyalkylamine, alkoxyoxycarboxylate, amino, alkylamino, dialkylamino, —O—N-methylacetamido, C₆₋₂₄ alkyl (e.g., n-C₆₋₂₄ alkyl), or —O—C₄₋₃₀alkyl-ON(CH₂R₈)(CH₂R₉), or —O—C₄₋₃₀alkyl-ON(CH₂R₈)(CH₂R₉). For example, R² is hydrogen, hydroxyl, protected hydroxyl, halogen, optionally substituted C₁₋₃₀ alkoxy, optionally substituted C₁₋₃₀ alkyl or alkoxyalkyl (e.g., methoxyethyl).

In some embodiments, R² is hydrogen, hydroxyl, protected hydroxyl, fluoro, methoxy, ethoxy, 2-methoxyethoxy, or —O—N-methylacetamido. For example, R² is hydrogen, hydroxyl, protected hydroxyl, fluoro or methoxy.

In some embodiments, R² is —OR²²², where R²²² is hydrogen, oxygen protecting group, optionally substituted C₁₋₃₀alkyl, C₁₋₃₀haloalkyl, optionally substituted C₂₋₃₀alkenyl, optionally substituted C₂₋₃₀alkynyl, or optionally substituted C₁₋₃₀alkoxy, cycloalkyl, heterocyclyl, aryl, heteroaryl.

In some embodiments, R²²² is hydrogen, i.e., R² is OH.

In some embodiments, R²²² is an oxygen protecting group, i.e., R² is —OR^{Pro}, where R^{Pro} is selected from the group consisting of acetyl, benzyl, t-butyldimethylsilyl, t-butyldiphenylsilyl, trimethylsilyl, triisopropylsilyl, and dimethoxytrityl. In some embodiments, R² is —OR^{Pro}, wherein R^{Pro} is selected from the group consisting of t-butyldimethylsilyl, t-butyldiphenylsilyl, trimethylsilyl, and triisopropylsilyl.

In some embodiments of any one of the aspects described herein, R² is a reactive phosphorus group. Optionally, only one of R² and R³ is a reactive phosphorous group.

Without wishing to be bound by a theory, reactive phosphorus groups are useful for forming internucleoside linkages including for example phosphodiester and phosphorothioate internucleoside linkages. Such reactive phosphorus groups are known in the art and contain phosphorus atoms in P^{III} or P^{V} valence state including, but not limited to, phosphoramidite, H-phosphonate, phosphate triesters and phosphorus containing chiral auxiliaries. Reactive phosphorous group in the form of phosphoramidites (P^{III} chemistry) as reactive phosphites are a preferred reactive phosphorous group for solid phase oligonucleotide synthesis. The intermediate phosphite compounds are subsequently oxidized to the Pv state using known methods to yield phosphodiester or phosphorothioate internucleoside linkages.

In some embodiments of any one of the aspects described herein, the reactive phosphorous group is —OP(OR^{P})(N(R^{P2})₂), —OP(SR^{P})(N(R^{P2})₂), —OP(O)(OR^{P})(N(R^{P2})₂), —OP(S)(OR^{P})(N(R^{P2})₂), —OP(O)(SR^{P})(N(R^{P2})₂), —OP(O)(OR^{P})H, —OP(S)(OR^{P})H, —OP(O)(SR^{P})H, —OP(O)(OR^{P})R^{P3}, —OP(S)(OR^{P})R^{P3}, or —OP(O)(SR^{P})R^{P3}. For example, the reactive phosphorous group is —OP(OR^{P})(N(R^{P2})₂).

In some embodiments of any one of the aspects, R^{P} is an optionally substituted C₁₋₆alkyl. For example, R^{P} is a C₁₋₆alkyl, optionally substituted with 1, 2, 3, 4 or 5 substituents independently selected from OH, CN, SC(O)Ph, oxo (═O), SH, SO₂NH₂, SO₂(C₁-C₄)alkyl, SO₂NH(C₁-C₄)alkyl, halogen, carbonyl, thiol, cyano, NH₂, NH(C₁-C₄)alkyl, N[(C₁-C₄)alkyl]₂, C(O)NH₂, COOH, COOMe, acetyl, (C₁-C₈)alkyl, O(C₁-C₈)alkyl (i.e., C₁-C₈alkoxy), O(C₁-C₈)haloalkyl, (C₂-C₈)alkenyl, (C₂-C₈)alkynyl, haloalkyl, thioalkyl, cyanomethylene, alkylaminyl, aryl, heteroaryl, substituted aryl, NH₂—C(O)-alkylene, NH(Me)-C(O)-alkylene, CH₂—C(O)-alkyl, C(O)-alkyl, alkylcarbonylaminyl, CH₂—[CH(OH)]ₘ—(CH₂)ₚ—OH, CH₂—[CH(OH)]ₘ—(CH₂)ₚ—NH₂ or CH₂-aryl-alkoxy, where “m” and “p” are independently 1, 2, 3, 4, 5 or 6. In some embodiments, R^{p} is a C₁₋₆alkyl, optionally substituted with a CN or —SC(O)Ph. For example, R^{P} is cyanoethyl (—CH₂CH₂CN).

In the reactive phosphorous groups, each R^{P2} is independently optionally substituted C₁₋₆alkyl. For example, each R^{P2} can be independently selected from methyl, ethyl, propyl, isopropyl, n-butyl, iso-butyl, pentyl or hexyl. It is noted that when two or more R^{P2} groups are present in the reactive phosphorous group, they can be same or different. Thus, in some none-limiting examples, when two or more R^{P2} groups are present, the R^{P2} groups are different. In some other non-limiting examples, when two or more R^{P2} groups are present, the R^{P2} groups are same. In some embodiments of any one of the aspects, each R^{P2} is isopropyl.

In some embodiments of any one of the aspects, both R^{P2} taken together with the nitrogen atom to which they are attached form an optionally substituted 3-8 membered heterocyclyl. Exemplary heterocyclyls include, but are not limited to, pyrrolidinyl, piperazinyl, dioxanyl, morpholinyl, tetrahydrofuranyl, piperidyl, 4-morpholyl, 4-piperazinyl, pyrrolidinyl, perhydropyrrolizinyl, 1,4-diazaperhydroepinyl, 1,3-dioxanyl, 1,4-dioxanyl and the like, each of which can be optionally substituted with 1, 2 or 3 substituents independently selected from OH, CN, SC(O)Ph, oxo (═O), SH, SO₂NH₂, SO₂(C₁-C₄)alkyl, SO₂NH(C₁-C₄)alkyl, halogen, carbonyl, thiol, cyano, NH₂, NH(C₁-C₄)alkyl, N[(C₁-C₄)alkyl]₂, C(O)NH₂, COOH, COOMe, acetyl, (C₁-C₈)alkyl, O(C₁-C₈)alkyl (i.e., C₁-C₈alkoxy), O(C₁-C₈)haloalkyl, (C₂-C₈)alkenyl, (C₂-C₈)alkynyl, haloalkyl, thioalkyl, cyanomethylene, alkylaminyl, aryl, heteroaryl, substituted aryl, NH₂—C(O)-alkylene, NH(Me)-C(O)-alkylene, CH₂—C(O)-alkyl, C(O)-alkyl, alkylcarbonylaminyl, CH₂—[CH(OH)]ₘ—(CH₂)ₚ—OH, CH₂—[CH(OH)]ₘ—(CH₂)ₚ—NH₂ or CH₂-aryl-alkoxy, where “m” and “p” are independently 1, 2, 3, 4, 5 or 6.

In some embodiments of any one of the aspects, R^{P} and one of R^{P2} taken together with the atoms to which they are attached form an optionally substituted 4-8 membered heterocyclyl. Exemplary heterocyclyls include, but are not limited to, pyrrolidinyl, piperazinyl, dioxanyl, morpholinyl, tetrahydrofuranyl, piperidyl, 4-morpholyl, 4-piperazinyl, pyrrolidinyl, perhydropyrrolizinyl, 1,4-diazaperhydroepinyl, 1,3-dioxanyl, 1,4-dioxanyl and the like, each of which can be optionally substituted with 1, 2 or 3 substituents independently selected from OH, CN, SC(O)Ph, oxo (═O), SH, SO₂NH₂, SO₂(C₁-C₄)alkyl, SO₂NH(C₁-C₄)alkyl, halogen, carbonyl, thiol, cyano, NH₂, NH(C₁-C₄)alkyl, N[(C₁-C₄)alkyl]₂, C(O)NH₂, COOH, COOMe, acetyl, (C₁-C₈)alkyl, O(C₁-C₈)alkyl (i.e., C₁-C₈alkoxy), O(C₁-C₈)haloalkyl, (C₂-C₈)alkenyl, (C₂-C₈)alkynyl, haloalkyl, thioalkyl, cyanomethylene, alkylaminyl, aryl, heteroaryl, substituted aryl, NH₂—C(O)-alkylene, NH(Me)-C(O)-alkylene, CH₂—C(O)-alkyl, C(O)-alkyl, alkylcarbonylaminyl, CH₂—[CH(OH)]ₘ—(CH₂)ₚ—OH, CH₂—[CH(OH)]ₘ—(CH₂)ₚ—NH₂ or CH₂-aryl-alkoxy, where “m” and “p” are independently 1, 2, 3, 4, 5 or 6.

In the reactive phosphorous groups, each R^{P3} is independently optionally substituted C₁₋₆alkyl. For example, R^{P3} can be a C₁₋₆alkyl, optionally substituted with 1, 2, 3, 4 or 5 substituents independently selected from OH, CN, SC(O)Ph, oxo (═O), SH, SO₂NH₂, SO₂(C₁-C₄)alkyl, SO₂NH(C₁-C₄)alkyl, halogen, carbonyl, thiol, cyano, NH₂, NH(C₁-C₄)alkyl, N[(C₁-C₄)alkyl]₂, C(O)NH₂, COOH, COOMe, acetyl, (C₁-C₈)alkyl, O(C₁-C₈)alkyl (i.e., C₁-C₈alkoxy), O(C₁-C₈)haloalkyl, (C₂-C₈)alkenyl, (C₂-C₈)alkynyl, haloalkyl, thioalkyl, cyanomethylene, alkylaminyl, aryl, heteroaryl, substituted aryl, NH₂—C(O)-alkylene, NH(Me)-C(O)-alkylene, CH₂—C(O)-alkyl, C(O)-alkyl, alkylcarbonylaminyl, CH₂—[CH(OH)]ₘ—(CH₂)ₚ—OH, CH₂—[CH(OH)]ₘ—(CH₂)ₚ—NH₂ or CH₂-aryl-alkoxy, where “m” and “p” are independently 1, 2, 3, 4, 5 or 6. For example, R^{P3} is methyl, ethyl, propyl, isopropyl, n-butyl, iso-butyl, pentyl or hexyl, each of which can be optionally substituted with a NH₂, OH, C(O)NH₂, COOH, halo, SH, or C₁-C₆alkoxy.

In some embodiments of any one of the aspects, the reactive phosphorous group is —OP(OR^{P})(N(R^{P2})₂). For example, the reactive phosphorous group is —OP(OR^{P})(N(R^{P2})₂), where R^{P} is cyanoethyl (—CH₂CH₂CN) and each R^{P2} is isopropyl.

In some embodiments of any one of the aspects described herein, R² is —OP(OR^{P})(N(R^{P2})₂), —OP(SR^{P})(N(R^{P2})₂), —OP(O)(OR^{P})(N(R^{P2})₂), —OP(S)(OR^{P})(N(R^{P2})₂), —OP(O)(SR^{P})(N(R^{P2})₂), —OP(O)(OR^{P})H, —OP(S)(OR^{P})H, —OP(O)(SR^{P})H, —OP(O)(OR^{P})R^{P3}, —OP(S)(OR^{P})R^{P3}, or —OP(O)(SR^{P})R^{P3}.

In some embodiments of any one of the aspects, R² is —OP(OR^{P}) (N(R^{P2})₂), —OP(SR^{P})(N(R^{P2})₂), —OP(O)(OR^{P})(N(R^{P2})₂), —OP(S)(OR^{P})(N(R^{P2})₂), —OP(O)(SR^{P})(N(R^{P2})₂), —OP(O)(OR^{P})H, —OP(S)(OR^{P}) an optionally substituted C₁₋₆alkyl, each R^{P2} is independently optionally substituted C₁₋₆alkyl; and each R^{P3} is independently optionally substituted C₁₋₆alkyl.

In some embodiments, R² is [(2-cyanoethyl)-(N,N-diisopropyl)]-phosphoramidite or [(ß-thiobenzoylethyl)-(1-pyrrolidinyl)]-thiophosphoramidite.

In some embodiments of any one of the aspects, R² is —OP(OR^{P})(N(R^{P2})₂). For example, the R² is —OP(OR^{P})(N(R^{P2})₂), where R^{P} is cyanoethyl (—CH₂CH₂CN) and each R^{P2} is isopropyl.

In some embodiments, R² is —OP(SR^{P})(N(R^{P2})₂). For example, R² is —OP(SR^{P})(N(R^{P2})₂), where R^{P} is β-thiobenzoylethyl and the two R^{P2}, together with the nitrogen they are attached to form a pyrrolidine.

In some embodiments of any one of the aspects described herein, R² is a solid support or a linker covalently attached to a solid support. For example, R² is —OC(O)CH₂CH₂C(O)NH—Z, where Z is a solid support. In some embodiments, R² is —OC(O)CH₂CH₂CO₂H.

It is noted that only one of R² and R³ can be a linker attached covalently to a solid support.

### R³

In some embodiments of any one of the aspects described herein, R³ is —O(CH₂)ₘ₁—X′—R^{M′}, —O(CH₂)ₙ₁—C(O)N(R^{N′})(R^{N″}), hydrogen, hydroxyl, protected hydroxyl, phosphate group, reactive phosphorous group, halogen, optionally substituted C₁₋₃₀ alkyl, optionally substituted C₂₋₃₀alkenyl, optionally substituted C₂₋₃₀alkynyl, optionally substituted C₁₋₃₀ alkoxy (e.g., methoxy, 2-methoxyethoxy), alkoxyalkyl (e.g., methoxyethyl such as 2-methoxyethyl), alkoxyalkylamine, alkoxyoxycarboxylate, amino, alkylamino, dialkylamino, protected aminoalkyl, —O—N-methylacetamido, —O—C₄₋₃₀alkyl-ON(CH₂R⁸)(CH₂R⁹), or —O—C₄₋₃₀alkyl-ON(CH₂R⁸)(CH₂R⁹), a solid support, a linker or a linker covalently attached to a solid support. For example, R³ is —O(CH₂)ₘ₁—X′—R^{M′}, —O(CH₂)ₙ₁—C(O)N(R^{N′})(R^{N″}), hydrogen, hydroxyl, protected hydroxyl, phosphate group, reactive phosphorous group, halogen, optionally substituted C₁₋₃₀ alkoxy, alkoxyalkyl (e.g., methoxyethyl), alkoxyalkylamine, alkoxyoxycarboxylate, amino, alkylamino, dialkylamino, —O—N-methylacetamido, or C₆₋₂₄ alkyl (e.g., n-C₆₋₂₄ alkyl).

In some embodiments of the any one of the aspects described herein, R³ is —O(CH₂)ₘ₁—X^{M′}—R^{M′}. For example, R³ is —O(CH₂)ₘ₁—O—R^{M′}. In another non-limiting example, R³ is —O(CH₂)ₘ₁—S—R^{M′}.

In some embodiments of any one of the aspects, R³ is —OCH₂CH₂—X′—R^{M′}. For example, R³ is —OCH₂CH₂—O—R^{M′}. In another non-limiting example, R³ is —OCH₂CH₂—S—R^{M′}.

In some embodiments of any one the aspects described herein, R³ is —O(CH₂)ₙ₁—C(O)N(R^{N′})(R^{N″}). It is noted, when R³ is —O(CH₂)ₙ₁—C(O)N(R^{N′})(R^{N″}) then n1 can be 1 or 2. Accordingly, in some embodiments of any one the aspects described herein, R³ is —OCH₂—C(O)N(R^{N′})(R^{N″}). In some other embodiments of any one of the aspects described herein, R³ is —OCH₂CH₂—C(O)N(R^{N′})(R^{N″}).

In some embodiments of any one of the aspects, R³ is hydrogen, hydroxyl, halogen, protected hydroxyl, optionally substituted C₁₋₃₀ alkyl, optionally substituted C₂₋₃₀ alkenyl, optionally substituted C₂₋₃₀alkynyl, optionally substituted C₁₋₃₀alkoxy (e.g., methoxy, 2-methoxyethoxy), alkoxyalkyl (e.g., methoxyethyl such a 2-methoxyethyl), alkoxyalkylamine, alkoxyoxycarboxylate, amino, alkylamino, dialkylamino, —O—N-methylacetamido, C₆₋₂₄ alkyl (e.g., n-C₆₋₂₄ alkyl), or —O—C₄₋₃₀alkyl-ON(CH₂R₈)(CH₂R₉), or —O—C₄₋₃₀alkyl-ON(CH₂R₈)(CH₂R₉). For example, R³ is hydrogen, hydroxyl, protected hydroxyl, halogen, optionally substituted C₁₋₃₀ alkoxy, optionally substituted C₁₋₃₀ alkyl or alkoxyalkyl (e.g., methoxyethyl).

In some embodiments, R³ is hydrogen, hydroxyl, protected hydroxyl, fluoro, methoxy, ethoxy, 2-methoxyethoxy, or —O—N-methylacetamido. For example, R² is hydrogen, hydroxyl, protected hydroxyl, fluoro or methoxy.

In some embodiments, R³ is —OR²²², where R²²² is hydrogen, oxygen protecting group, optionally substituted C₁₋₃₀alkyl, C₁₋₃₀haloalkyl, optionally substituted C₂₋₃₀alkenyl, optionally substituted C₂₋₃₀alkynyl, or optionally substituted C₁₋₃₀alkoxy, cycloalkyl, heterocyclyl, aryl, heteroaryl.

In some embodiments, R²²² is hydrogen, i.e., R³ is OH

In some embodiments, R²²² is an oxygen protecting group, i.e., R³ is —OR^{Pro}, where R^{Pro} is selected from the group consisting of acetyl, benzyl, t-butyldimethylsilyl, t-butyldiphenylsilyl, trimethylsilyl, triisopropylsilyl, and dimethoxytrityl. In some embodiments, R³ is —OR^{Pro}, wherein R^{Pro} is selected from the group consisting of t-butyldimethylsilyl, t-butyldiphenylsilyl, trimethylsilyl, and triisopropylsilyl.

In some embodiments of any one of the aspects described herein, R³ is a reactive phosphorus group. Optionally, only one of R² and R³ is a reactive phosphorous group.

In some embodiments, R³ is [(2-cyanoethyl)-(N,N-diisopropyl)]-phosphoramidite or [(ß-thiobenzoylethyl)-(1-pyrrolidinyl)]-thiophosphoramidite.

In some embodiments of any one of the aspects, R³ is —OP(OR^{P})(N(R^{P2})₂). For example, the R³ is —OP(OR^{P})(N(R^{P2})₂), where R^{P} is cyanoethyl (—CH₂CH₂CN) and each R² is isopropyl.

In some embodiments, R³ is —OP(SR^{P})(N(R^{P2})₂). For example, R³ is —OP(SR^{P})(N(R^{P2})₂), where R^{P} is β-thiobenzoylethyl and the two R^{P2}, together with the nitrogen they are attached to form a pyrrolidine.

In some embodiments of any one of the aspects described herein, R³ is a solid support or a linker covalently attached to a solid support. For example, R³ is —OC(O)CH₂CH₂C(O)NH—Z, where Z is a solid support. In some embodiments, R³ is —OC(O)CH₂CH₂CO₂H. It is noted that only one of R² and R³ can be a linker attached covalently to a solid support.

In some embodiments, one of R² and R³ is —OCH₂CH₂—X^{M′}—R^{M′} or —O(CH₂)ₙ₁—C(O)N(R^{N′})(R^{N″}), and the other of R² and R³ is hydrogen, hydroxyl, protected hydroxyl, halogen, optionally substituted C₁₋₃₀ alkoxy, a reactive phosphorous group, a solid support, a linker, or a linker covalently attached to a solid support. For example, one of R² and R³ is —OCH₂CH₂—X^{M′}—R^{M′} or —O(CH₂)ₙ₁—C(O)N(R^{N′})(R^{N″}), and the other of R² and R³ is hydrogen, hydroxyl, protected hydroxyl, a reactive phosphorous group, a linker, or a linker covalently attached to a solid support.

In some embodiment, R² is —O(CH₂)ₘ₁—X^{M′}—R^{M′} (e.g., —OCH₂CH₂—X^{M′}—R^{M′}) or —O(CH₂)ₙ₁—C(O)N(R^{N′})(R^{N″}), and R³ is hydrogen, hydroxyl, protected hydroxyl, a reactive phosphorous group, a linker, or a linker covalently attached to a solid support.

In some other embodiments, R³ is —O(CH₂)ₘ₁—X^{M′}—R^{M′} (e.g., —OCH₂CH₂—X^{M′}—R^{M′}) or —O(CH₂)ₙ₁—C(O)N(R^{N′})(R^{N″}), and R² is hydrogen, hydroxyl, protected hydroxyl, a reactive phosphorous group, a linker, or a linker covalently attached to a solid support.

### R⁵

In some embodiments of any one of the various aspects described herein, R²⁵ is —O(CH₂)ₘ₁—X^{M′}—R^{M′}, —O(CH₂)ₙ₁—C(O)N(R^{N′})(R^{N″}), hydroxyl, protected hydroxyl, optionally substituted C₁₋₃₀ alkoxy, vinylphosphonate (VP) group, monophosphate, diphosphate, triphosphate, monothiophosphate (phosphorothioate), monodithiophosphate, phosphorothiolate, alpha-thiotriphosphate, beta-thiotriphosphate, gamma-thiotriphosphate, phosphoramidate, alkylphosphonate, alkyletherphosphonate, dialkyl terminal phosphate or phosphate mimic. For example, R²⁵ is —O(CH₂)ₘ₁—X^{M′}—R^{M′}, —O(CH₂)ₙ₁—C(O)N(R^{N′})(R^{N″}), hydroxyl, protected hydroxyl, vinylphosphonate (VP) group, cyclopropylphosphonate, monophosphate, diphosphate, triphosphate, monothiophosphate (phosphorothioate), monodithiophosphate, phosphorothiolate, alpha-thiotriphosphate, beta-thiotriphosphate, gamma-thiotriphosphate, phosphoramidates, alkylphosphonate, alkyletherphosphonate, dialkyl terminal phosphate, or a phosphate mimic. In some embodiments of any one of the various aspects described herein, R²⁵ is a vinylphosphonategroup, cyclopropylphosphonate. In some other embodiments, R²⁵ is hydroxyl or protected hydroxyl.

In some embodiments of the any one of the aspects described herein, R⁵ is —O(CH₂)ₘ₁—X^{M′}—R^{M′}. For example, R⁵ is —O(CH₂)ₘ₁—O—R^{M′}. In another non-limiting example, R⁵ is —O(CH₂)ₘ₁—S—R^{M′}.

In some embodiments of any one of the aspects, R⁵ is —OCH₂CH₂—X′—R^{M′}. For example, R⁵ is —OCH₂CH₂—O—R^{M′}. In another non-limiting example, R⁵ is —OCH₂CH₂—S—R^{M′}.

In some embodiments of any one the aspects described herein, R⁵ is —O(CH₂)ₙ₁—C(O)N(R^{N′})(R^{N″}). It is noted, when R⁵ is —O(CH₂)ₙ₁—C(O)N(R^{N′})(R^{N″}) then n1 can be 1 or 2. Accordingly, in some embodiments of any one the aspects described herein, R⁵ is —OCH₂—C(O)N(R^{N′})(R^{N″}). In some other embodiments of any one of the aspects described herein, R³ is —OCH₂CH₂—C(O)N(R^{N′})(R^{N″}).

In some embodiments of the any one of the aspects described herein, R⁵ is R⁵⁵¹, optionally substituted C₁₋₆alkyl-R⁵⁵¹, optionally substituted —C₂₋₆alkenyl-R⁵⁵¹, or optionally substituted —C₂₋₆alkynyl-R⁵⁵¹, where R⁵⁵¹ can be —OR⁵⁵², —SR⁵⁵³, hydrogen, a phosphorous group, a solid support or a linker to a solid support. When R⁵⁵¹ is —OR⁵⁵², R⁵⁵² can be hydrogen, oxygen protecting group, optionally substituted C₁₋₃₀alkyl, C₁₋₃₀haloalkyl, optionally substituted C₂₋₃₀alkenyl, optionally substituted C₂₋₃₀alkynyl, or optionally substituted C₁₋₃₀alkoxy, cycloalkyl, heterocyclyl, aryl, heteroaryl. Similarly, when R⁵⁵¹ is —SR⁵⁵³, R⁵⁵³ can be hydrogen, sulfur protecting group, optionally substituted C₁₋₃₀alkyl, C₁₋₃₀haloalkyl, optionally substituted C₂₋₃₀alkenyl, optionally substituted C₂₋₃₀alkynyl, or optionally substituted C₁₋₃₀alkoxy, cycloalkyl, heterocyclyl, aryl, heteroaryl.

In some embodiments of any one of the aspects described herein, R⁵ is —OR⁵⁵², where is hydrogen or oxygen protecting group. Exemplary hydroxyl protecting groups for R⁵⁵² include, but are not limited to, benzyl, benzoyl, 2,6-dichlorobenzyl, t-butyldimethylsilyl, t-butyldiphenylsilyl, mesylate, tosylate, 4,4′-dimethoxytrityl, 9-phenylxanthine-9-yl (Pixyl) and 9-(p-methoxyphenyl)xanthine-9-yl (MOX).

In some embodiments of any one of the various aspects described herein, R⁵⁵² is hydrogen, i.e., R⁵ is OH.

In some embodiments of any one of the various aspects described herein, R⁵⁵² is an oxygen protecting group, i.e., R⁵ is —OR^{Pro}, where R^{Pro} is an oxygen protecting group. For example, R⁵ is —OR^{Pro}, where R^{Pro} is selected from the group consisting of acetyl, benzyl, t-butyldimethylsilyl, t-butyldiphenylsilyl, trimethylsilyl, triisopropylsilyl, and dimethoxytrityl. In some embodiments of any one of the various aspects described herein, R⁵ is —OR^{Pro}, wherein R^{Pro} is selected from the group consisting of t-butyldimethylsilyl, t-butyldiphenylsilyl, trimethylsilyl, and triisopropylsilyl.

In some embodiments of any one of the various aspects described herein, R⁵ is —OR^{Pro}, where R^{Pro} is 4,4′-dimethoxytrityl (DMT).

In some embodiments of any one of the various aspects described herein, one of R² and R³ is —O(CH₂)ₘ₁—X^{M′}—R^{M′} (e.g., —OCH₂CH₂—X^{M′}—R^{M′}) or —O(CH₂)ₙ₁—C(O)N(R^{N′})(R^{N″}), the other of R² and R³ is hydrogen, hydroxyl, protected hydroxyl, halogen, optionally substituted C₁₋₃₀ alkoxy, a reactive phosphorous group, a solid support, a linker, or a linker covalently attached to a solid support, and R⁵ is hydroxyl or —OR^{Pro}, optionally R^{Pro} is DMT. For example, one of R² and R³ is —O(CH₂)ₘ₁—X^{M′}_R^{M′} (e.g., —OCH₂CH₂—X^{M′}_R^{M′}) or —O(CH₂)ₙ₁—C(O)N(R^{N′})(R^{N″}), the other of R² and R³ is hydrogen, hydroxyl, protected hydroxyl, a reactive phosphorous group, a linker, or a linker covalently attached to a solid support, and R⁵ is hydroxyl or —OR^{Pro}, optionally R^{Pro} is DMT.

In some embodiment, R² is —O(CH₂)ₘ₁—X^{M′}—R^{M′} (e.g., —OCH₂CH₂—X^{M′}—R^{M′}) or —O(CH₂)ₙ₁—C(O)N(R^{N′})(R^{N″}), and R³ is hydrogen, hydroxyl, protected hydroxyl, a reactive phosphorous group, a linker, or a linker covalently attached to a solid support, and R⁵ is hydroxyl or —OR^{Pro}, optionally R^{Pro} is DMT.

In some other embodiments, R³ is —O(CH₂)ₘ₁—X^{M′}—R^{M′} (e.g., —OCH₂CH₂—X^{M′}—R^{M′}) or —O(CH₂)ₙ₁—C(O)N(R^{N′})(R^{N″}), and R³ is hydrogen, hydroxyl, protected hydroxyl, a reactive phosphorous group, a linker, or a linker covalently attached to a solid support, and R⁵ is hydroxyl or —OR^{Pro}, optionally R^{Pro} is DMT.

In some embodiments of any one of the various aspects described herein, one of R² and R³ is —O(CH₂)ₘ₁—X^{M′}—R^{M′} (e.g., —OCH₂CH₂—X^{M′}—R^{M′}) or —O(CH₂)ₙ₁—C(O)N(R^{N′})(R^{N″}), the other of R² and R³ is hydrogen, hydroxyl, protected hydroxyl, halogen, optionally substituted C₁₋₃₀ alkoxy, a reactive phosphorous group, a solid support, a linker, or a linker covalently attached to a solid support, and R⁵ is hydroxyl or —OR^{Pro}, optionally R^{Pro} is DMT. For example, one of R² and R³ is —O(CH₂)ₘ₁—X^{M′}—R^{M′} (e.g., —OCH₂CH₂—X^{M′}—R^{M′}) or —O(CH₂)ₙ₁—C(O)N(R^{N′})(R^{N″}), the other of R² and R³ is hydrogen, hydroxyl, protected hydroxyl, a reactive phosphorous group, a linker, or a linker covalently attached to a solid support, and R⁵ is hydroxyl or —OR^{Pro}, optionally R^{Pro} is DMT.

In some embodiment, R² is —O(CH₂)ₘ₁—X^{M′}—R^{M′} (e.g., —OCH₂CH₂—X^{M′}—R^{M′}) or —O(CH₂)ₙ₁—C(O)N(R^{N′})(R^{N″}), and R³ is hydrogen, hydroxyl, protected hydroxyl, a reactive phosphorous group, a linker, or a linker covalently attached to a solid support, and R⁵ is vinylphosphonate (VP) group, monophosphate, diphosphate, triphosphate, monothiophosphate (phosphorothioate), monodithiophosphate, phosphorothiolate, alpha-thiotriphosphate, beta-thiotriphosphate, gamma-thiotriphosphate, phosphoramidate, alkylphosphonate, alkyletherphosphonate, dialkyl terminal phosphate or phosphate mimic, optionally R^{Pro} is a vinylphosphate group.

In some other embodiments, R³ is —O(CH₂)ₘ₁—X^{M′}—R^{M′} (e.g., —OCH₂CH₂—X^{M′}—R^{M′}) or —O(CH₂)ₙ₁—C(O)N(R^{N′})(R^{N″}), and R³ is hydrogen, hydroxyl, protected hydroxyl, a reactive phosphorous group, a linker, or a linker covalently attached to a solid support, and R⁵ is vinylphosphonate (VP) group, monophosphate, diphosphate, triphosphate, monothiophosphate (phosphorothioate), monodithiophosphate, phosphorothiolate, alpha-thiotriphosphate, beta-thiotriphosphate, gamma-thiotriphosphate, phosphoramidate, alkylphosphonate, alkyletherphosphonate, dialkyl terminal phosphate or phosphate mimic, optionally R^{Pro} is a vinylphosphate group.

In some embodiments of any one of the aspects described herein, the methylene connecting the R⁵ to the rest of the compound of Formula (I) is absent and R⁵ is connected directly to the rest of the compound of Formula (I).

In some embodiments of any one of the aspects described herein, R⁵ is —CH(R⁵⁵⁴)—R⁵⁵¹, where R⁵⁵⁴ is hydrogen, halogen, optionally substituted C₁-C₃₀alkyl, optionally substituted C₂-C₃₀alkenyl, optionally substituted C₂-C₃₀alkynyl, or optionally substituted C₁-C₃₀alkoxy.

In some embodiments of any one of the aspects, when R⁵ is —CH(R⁵⁵⁴)—R⁵⁵¹, R⁵⁵⁴ is H or C₁-C₃₀alkyl optionally substituted with 1, 2, 3, 4 or 5 substituents independently selected from OH, CN, SC(O)Ph, oxo (═O), SH, SO₂NH₂, SO₂(C₁-C₄)alkyl, SO₂NH(C₁-C₄)alkyl, halogen, carbonyl, thiol, cyano, NH₂, NH(C₁-C₄)alkyl, N[(C₁-C₄)alkyl]₂, C(O)NH₂, COOH, COOMe, acetyl, (C₁-C₈)alkyl, O(C₁-C₈)alkyl (i.e., C₁-C₈alkoxy), O(C₁-C₈)haloalkyl, (C₂-C₈)alkenyl, (C₂-C₈)alkynyl, haloalkyl, thioalkyl, cyanomethylene, alkylaminyl, aryl, heteroaryl, substituted aryl, NH₂—C(O)-alkylene, NH(Me)-C(O)-alkylene, CH₂—C(O)-alkyl, C(O)-alkyl, alkylcarbonylaminyl, CH₂—[CH(OH)]ₘ—(CH₂)ₚ—OH, CH₂—[CH(OH)]ₘ—(CH₂)ₚ—NH₂ or CH₂-aryl-alkoxy, where “m” and “p” are independently 1, 2, 3, 4, 5 or 6. For example, R⁵⁵⁴ is H. In some other non-limiting examples, R⁵⁵⁴ is C₁-C₃₀alkyl optionally substituted with a NH₂, OH, C(O)NH₂, COOH, halo, SH, or C₁-C₆alkoxy.

In some embodiments of the any one of the aspects described herein, R⁵ is —CH(R⁵⁵⁴)—O—R⁵⁵², where R⁵⁵⁴ is H or C₁-C₃₀alkyl optionally substituted with 1, 2, 3, 4 or 5 substituents independently selected from OH, CN, SC(O)Ph, oxo (═O), SH, SO₂NH₂, SO₂(C₁-C₄)alkyl, SO₂NH(C₁-C₄)alkyl, halogen, carbonyl, thiol, cyano, NH₂, NH(C₁-C₄)alkyl, N[(C₁-C₄)alkyl]₂, C(O)NH₂, COOH, COOMe, acetyl, (C₁-C₈)alkyl, O(C₁-C₈)alkyl (i.e., C₁-C₈alkoxy), O(C₁-C₈)haloalkyl, (C₂-C₈)alkenyl, (C₂-C₈)alkynyl, haloalkyl, thioalkyl, cyanomethylene, alkylaminyl, aryl, heteroaryl, substituted aryl, NH₂—C(O)-alkylene, NH(Me)-C(O)-alkylene, CH₂—C(O)-alkyl, C(O)-alkyl, alkylcarbonylaminyl, CH₂—[CH(OH)]ₘ—(CH₂)ₚ—OH, CH₂—[CH(OH)]ₘ—(CH₂)ₚ—NH₂ or CH₂-aryl-alkoxy, where “m” and “p” are independently 1, 2, 3, 4, 5 or 6. For example, R⁵⁵⁴ is H. In some other non-limiting examples, R⁵⁵⁴ is C₁-C₃₀alkyl optionally substituted with a NH₂, OH, C(O)NH₂, COOH, halo, SH, or C₁-C₆alkoxy.

In some embodiments of the any one of the aspects described herein, R⁵ is optionally substituted C₁₋₆alkyl-R⁵⁵¹ or optionally substituted —C₂₋₆alkenyl-R⁵⁵¹,

In some embodiments of any one of the aspects described herein, R⁵ is —C(R⁵⁵⁴)═CR⁵⁵¹. It is noted that the double bond in —C(R⁵⁵⁴)═CHR⁵⁵¹ can be in the cis or trans configuration. Accordingly, in some embodiments of any one of the aspects, R^{d} is —C(R⁵⁵⁴)═CHR⁵⁵¹ and wherein the double bond is in the cis configuration. In some other embodiments of any one of the aspects, R^{d} is —C(R⁵⁵⁴)═CHR⁵⁵¹ and wherein the double bond is in the trans configuration.

In some embodiments of any one of the aspects described herein, R⁵ is —CH═CHR⁵⁵¹.

In some embodiments of any one of the aspects, when R⁵ is —C(R⁵⁵⁴)═CH₁R⁵⁵¹, R⁵⁵⁴ is H or C₁-C₃₀alkyl optionally substituted with 1, 2, 3, 4 or 5 substituents independently selected from OH, CN, SC(O)Ph, oxo (═O), SH, SO₂NH₂, SO₂(C₁-C₄)alkyl, SO₂NH(C₁-C₄)alkyl, halogen, carbonyl, thiol, cyano, NH₂, NH(C₁-C₄)alkyl, N[(C₁-C₄)alkyl]₂, C(O)NH₂, COOH, COOMe, acetyl, (C₁-C₈)alkyl, O(C₁-C₈)alkyl (i.e., C₁-C₈alkoxy), O(C₁-C₈)haloalkyl, (C₂-C₈)alkenyl, (C₂-C₈)alkynyl, haloalkyl, thioalkyl, cyanomethylene, alkylaminyl, aryl, heteroaryl, substituted aryl, NH₂—C(O)-alkylene, NH(Me)-C(O)-alkylene, CH₂—C(O)-alkyl, C(O)-alkyl, alkylcarbonylaminyl, CH₂—[CH(OH)]ₘ—(CH₂)ₚ—OH, CH₂—[CH(OH)]ₘ—(CH₂)ₚ—NH₂ or CH₂-aryl-alkoxy, where “m” and “p” are independently 1, 2, 3, 4, 5 or 6; and R⁵⁵¹ is a phosphorous group. For example, R⁵ is —CH═CHR⁵⁵¹.

In some embodiments of any one of the aspects described herein, R⁵⁵¹ is a reactive phosphorous group.

In some embodiments of any one of the aspects, R⁵ is —CH═CH—P(O)(OR⁵⁵⁵)₂, —CH═CH—P(S)(OR⁵⁵⁵)₂, —CH═CH—P(S)(SR⁵⁵⁶)(OR⁵⁵⁵), —CH═CH—P(S)(SR⁵⁵⁶)₂, —CH═CH—OP(O)(OR⁵⁵⁵)₂, —CH═CH—OP(S)(OR⁵⁵⁵)₂, —CH═CH—OP(S)(SR⁵⁵⁶)(OR⁵⁵⁵), —CH═CH—OP(S)(SR⁵⁵⁶)₂, —CH═CH—SP(O)(OR⁵⁵⁵)₂, —CH═CH—SP(S)(OR⁵⁵⁵)₂, —CH═CH—SP(S)(SR⁵⁵⁶)(OR⁵⁵), or —CH═CH—SP(S)(SR⁵⁵⁶)₂, where each R⁵⁵⁵ is independently hydrogen, optionally substituted C₁₋₃₀alkyl, optionally substituted C₂₋₃₀alkenyl, or optionally substituted C₂₋₃₀alkynyl, or an oxygen-protecting group; and each R⁵⁵⁶ is independently hydrogen, optionally substituted C₁₋₃₀alkyl, optionally substituted C₂₋₃₀alkenyl, or optionally substituted C₂₋₃₀alkynyl, or a sulfur-protecting group.

In some embodiments of any one of the aspects, at least one R⁵⁵⁵ in —P(O)(OR⁵⁵⁵)₂, —P(S)(OR⁵⁵⁵)₂, —P(S)(SR⁵⁵⁶)(OR⁵⁵⁵), —OP(O)(OR⁵⁵⁵)₂, —OP(S)(OR⁵⁵⁵)₂, —OP(S)(SR⁵⁵⁶)(OR⁵⁵⁵), SP(O)(OR⁵⁵⁵)₂, —SP(S)(OR⁵⁵⁵)₂, and —SP(S)(SR⁵⁵⁶)(OR⁵⁵⁵) is hydrogen.

In some other embodiments of any one of the aspects, at least one R⁵⁵⁵ in —P(O)(OR⁵⁵⁵)₂, —P(S)(OR⁵⁵⁵)₂, —P(S)(SR⁵⁵⁶)(OR⁵⁵⁵), —OP(O)(OR⁵⁵⁵)₂, —OP(S)(OR⁵⁵⁵)₂, —OP(S)(SR⁵⁵⁶)(OR⁵⁵⁵), SP(O)(OR⁵⁵⁵)₂, —SP(S)(OR⁵⁵⁵)₂, or —SP(S)(SR⁵⁵⁶)(OR⁵⁵⁵) is not hydrogen. For example, at least one at least one R⁵⁵⁵ in P(O)(OR⁵⁵⁵)₂, —P(S)(OR⁵⁵⁵)₂, —P(S)(SR⁵⁵⁶)(OR⁵⁵⁵), —OP(O)(OR⁵⁵⁵)₂, —OP(S)(OR⁵⁵⁵)₂, —OP(S)(SR⁵⁵⁶)(OR⁵⁵⁵), SP(O)(OR⁵⁵⁵)₂, —SP(S)(OR⁵⁵⁵)₂, and —SP(S)(SR⁵⁵⁶)(OR⁵⁵⁵) is optionally substituted C₁₋₃₀alkyl, optionally substituted C₂₋₃₀alkenyl, or optionally substituted C₂₋₃₀alkynyl, or an oxygen-protecting group.

In some embodiments of any one of the aspects, at least one R⁵⁵⁵ is H and at least one R⁵⁵⁵ is other than H in —P(O)(OR⁵⁵⁵)₂, —P(S)(OR⁵⁵⁵)₂, —P(S)(SR⁵⁵⁶)(OR⁵⁵⁵), —OP(O)(OR⁵⁵⁵)₂, —OP(S)(OR⁵⁵⁵)₂, —OP(S)(SR⁵⁵⁶)(OR⁵⁵⁵), SP(O)(OR⁵⁵⁵)₂, —SP(S)(OR⁵⁵⁵)₂, and —SP(S)(SR⁵⁵⁶)(OR⁵⁵⁵).

In some embodiments of any one of the aspects, all R⁵⁵⁵ are H in —P(O)(OR⁵⁵⁵)₂, —P(S)(OR⁵⁵⁵)₂, —P(S)(SR⁵⁵⁶)(OR⁵⁵⁵), —OP(O)(OR⁵⁵⁵)₂, —OP(S)(OR⁵⁵⁵)₂, —OP(S)(SR⁵⁵⁶)(OR⁵⁵⁵), —OP(S)(SR⁵⁵⁶)₂, —SP(O)(OR⁵⁵⁵)₂, —SP(S)(OR⁵⁵⁵)₂, —SP(S)(SR⁵⁵⁶)(OR⁵⁵⁵), and —SP(S)(SR⁵⁵⁶)₂.

In some embodiments of any one of the aspects, all R⁵⁵⁵ are other than H in in —P(O)(OR⁵⁵⁵)₂, —P(S)(OR⁵⁵⁵)₂, —P(S)(SR⁵⁵⁶)(OR⁵⁵⁵), —OP(O)(OR⁵⁵⁵)₂, —OP(S)(OR⁵⁵⁵)₂, —OP(S)(SR⁵⁵⁶)(OR⁵⁵⁵), —OP(S)(SR⁵⁵⁶)₂, —SP(O)(OR⁵⁵⁵)₂, —SP(S)(OR⁵⁵⁵)₂, —SP(S)(SR⁵⁵⁶)(OR⁵⁵⁵), and —SP(S)(SR⁵⁵⁶)₂.

In some embodiments of any one of the aspects, at least one R⁵⁵⁶ in —P(S)(SR⁵⁵⁶)(OR⁵⁵⁵), —P(S)(SR⁵⁵⁶)₂, —OP(S)(OR⁵⁵⁵)₂, —OP(S)(SR⁵⁵⁶)(OR⁵⁵⁵), —OP(S)(SR⁵⁵⁶)₂, —SP(S)(SR⁵⁵⁶)(OR⁵⁵⁵), and —SP(S)(SR⁵⁵⁶)₂ is H.

In some embodiments of any one of the aspects, at least one R⁵⁵⁶ in —P(S)(SR⁵⁵⁶)(OR⁵⁵⁵), —P(S)(SR⁵⁵⁶)₂, —OP(S)(OR⁵⁵⁵)₂, —OP(S)(SR⁵⁵⁶)(OR⁵⁵⁵), —OP(S)(SR⁵⁵⁶)₂, —SP(S)(SR⁵⁵⁶)(OR⁵⁵⁵), and —SP(S)(SR⁵⁵⁶)₂ is other than H. For example, at least one R⁵⁵⁶ in P(S)(SR⁵⁵⁶)(OR⁵⁵⁵), —P(S)(SR⁵⁵⁶)₂, —OP(S)(OR⁵⁵⁵)₂, —OP(S)(SR⁵⁵⁶)(OR⁵⁵⁵), —OP(S)(SR⁵⁵⁶)₂, —SP(S)(SR⁵⁵⁶)(OR⁵⁵⁵), and —SP(S)(SR⁵⁵⁶)₂ is optionally substituted C₁₋₃₀alkyl, optionally substituted C₂₋₃₀alkenyl, or optionally substituted C₂₋₃₀alkynyl, or an sulfur-protecting group.

In some embodiments of any one of the aspects, at least one R⁵⁵⁶ is H and at least one R⁵⁵⁶ is other than H in —P(S)(SR⁵⁵⁶)₂, —OP(S)(SR⁵⁵⁶)₂ and —SP(S)(SR⁵⁵⁶)₂.

In some embodiments of any one of the various aspects described herein, all R⁵⁵⁶ are H in —P(S)(SR⁵⁵⁶)(OR⁵⁵⁵), —P(S)(SR⁵⁵⁶)₂, —OP(S)(OR⁵⁵⁵)₂, —OP(S)(SR⁵⁵⁶)(OR⁵⁵⁵), —OP(S)(SR⁵⁵⁶)₂, —SP(S)(SR⁵⁵⁶)(OR⁵⁵⁵), and —SP(S)(SR⁵⁵⁶)₂.

In some embodiments of any one of the various aspects described herein, all R⁵⁵⁶ are other than H in —P(S)(SR⁵⁵⁶)(OR⁵⁵⁵), —P(S)(SR⁵⁵⁶)₂, —OP(S)(OR⁵⁵⁵)₂, —OP(S)(SR⁵⁵⁶)(OR⁵⁵⁵), —OP(S)(SR⁵⁵⁶)₂, —SP(S)(SR⁵⁵⁶)(OR⁵⁵⁵), and —SP(S)(SR⁵⁵⁶)₂.

In some embodiments of any one of the aspects, R⁵ is —CH═CH—P(O)(OR⁵⁵⁵)₂, where each R⁵⁵⁵ is H or an oxygen protecting group.

### R²²

In nucleosides of Formula (II), R²² is —O(CH₂)ₘ₁—X′—R^{M′}, —O(CH₂)ₙ₁—C(O)N(R^{N′})(R^{N″}), hydrogen, hydroxyl, protected hydroxyl, halogen, optionally substituted C₁₋₃₀ alkyl, optionally substituted C₂₋₃₀alkenyl, optionally substituted C₂₋₃₀alkynyl, optionally substituted C₁₋₃₀ alkoxy (e.g., methoxy, 2-methoxyethoxy), alkoxyalkyl (e.g., 2-methoxyethyl), alkoxyalkylamine, alkoxyoxycarboxylate, amino, alkylamino, dialkylamino, protected aminoalkyl, 5-8 membered heterocyclyl, —O—C₄₋₃₀alkyl-ON(CH₂R⁸)(CH₂R⁹), —O—N-methylacetamido, —O—C₄₋₃₀alkyl-ON(CH₂R⁸)(CH₂R⁹), a bond to an internucleotide linkage to a subsequent nucleotide, a 3′-oligonucleotide capping group, a ligand, a linker covalently bonded to one or more ligands, a solid support, a linker or a linker covalently bonded a solid support.

In some embodiments of the any one of the aspects described herein, R²² is —O(CH₂)ₘ₁—X^{M′}—R^{M′}. For example, R²² is —O(CH₂)ₘ₁—O—R^{M′}. In another non-limiting example, R²² is —O(CH₂)ₘ₁—S—R^{M′}.

In some embodiments of any one of the aspects, R²² is —OCH₂CH₂—X′—R^{M′}. For example, R²² is —OCH₂CH₂—O—R^{M′}. In another non-limiting example, R²² is —OCH₂CH₂—S—R^{M′}.

In some embodiments of any one the aspects described herein, R²² is —O(CH₂)ₙ₁—C(O)N(R^{N′})(R^{N″}). It is noted, when R²² is —O(CH₂)ₙ₁—C(O)N(R^{N′})(R^{N″}) then n1 can be 1 or 2. Accordingly, in some embodiments of any one the aspects described herein, R²² is —OCH₂—C(O)N(R^{N′})(R^{N″}). In some other embodiments of any one of the aspects described herein, R²² is —OCH₂CH₂—C(O)N(R^{N′})(R^{N″}).

In some embodiments, R²² is hydrogen, hydroxyl, protected hydroxyl, halogen, optionally substituted C₁₋₃₀ alkyl, optionally substituted C₂₋₃₀alkenyl, optionally substituted C₂₋₃₀alkynyl, optionally substituted C₁₋₃₀ alkoxy (e.g., methoxy, 2-methoxyethoxy), alkoxyalkyl (e.g., 2-methoxyethyl), amino, alkylamino, dialkylamino, protected aminoalkyl, —O—C₄₋₃₀alkyl-ON(CH₂R₈)(CH₂R₉), —O—C₄₋₃₀alkyl-ON(CH₂R₈)(CH₂R₉), —O—N-methylacetamido, alkoxyoxycarboxylate, a solid support, a linker or a linker covalently attached to a solid support. For example, R²² is hydrogen, hydroxyl, halogen, protected hydroxyl, optionally substituted C₁₋₃₀ alkyl, optionally substituted C₂₋₃₀alkenyl, optionally substituted C₂₋₃₀alkynyl, optionally substituted C₁₋₃₀alkoxy (e.g., methoxy), alkoxyalkyl (e.g., methoxyethyl), alkoxyalkylamine, alkoxyoxycarboxylate, amino, alkylamino, dialkylamino, protected aminoalkyl, —O—N-methylacetamido, —O—C₄₋₃₀alkyl-ON(CH₂R₈)(CH₂R₉), or —O—C₄₋₃₀alkyl-ON(CH₂R₈)(CH₂R₉).

In some embodiments of any one of the aspects described herein, R²² is a bond to an internucleotide linkage to a subsequent nucleotide, a linker or a linker covalently attached to a solid support. For example, R²² is a bond to an internucleotide linkage to a subsequent nucleotide. R²³

In nucleosides of Formula (II), R²³ is —O(CH₂)ₘ₁—X′—R^{M′}, —O(CH₂)ₙ₁—C(O)N(R^{N′})(R^{N″}), hydrogen, hydroxyl, protected hydroxyl, halogen, optionally substituted C₁₋₃₀ alkyl, optionally substituted C₂₋₃₀alkenyl, optionally substituted C₂₋₃₀alkynyl, optionally substituted C₁₋₃₀ alkoxy (e.g., methoxy, 2-methoxyethoxy), alkoxyalkyl (e.g., 2-methoxyethyl), alkoxyalkylamine, alkoxyoxycarboxylate, amino, alkylamino, dialkylamino, protected aminoalkyl, 5-8 membered heterocyclyl, —O—C₄₋₃₀alkyl-ON(CH₂R⁸)(CH₂R⁹), —O—N-methylacetamido, —O—C₄₋₃₀alkyl-ON(CH₂R⁸)(CH₂R⁹), a bond to an internucleotide linkage to a subsequent nucleotide, a 3′-oligonucleotide capping group, a ligand, a linker covalently bonded to one or more ligands, a solid support, a linker or a linker covalently bonded a solid support.

In some embodiments of the any one of the aspects described herein, R²³ is —O(CH₂)ₘ₁—X^{M′}—R^{M′}. For example, R²³ is —O(CH₂)ₘ₁—O—R^{M′}. In another non-limiting example, R²³ is —O(CH₂)ₘ₁—S—R^{M′}.

In some embodiments of any one of the aspects, R²³ is —OCH₂CH₂—X′—R^{M′}. For example, R²³ is —OCH₂CH₂—O—R^{M′}. In another non-limiting example, R²³ is —OCH₂CH₂—S—R^{M′}.

In some embodiments of any one the aspects described herein, R²³ is —O(CH₂)ₙ₁—C(O)N(R^{N′})(R^{N″}). It is noted, when R²³ is —O(CH₂)ₙ₁—C(O)N(R^{N′})(R^{N″}) then n1 can be 1 or 2. Accordingly, in some embodiments of any one the aspects described herein, R²³ is —OCH₂—C(O)N(R^{N′})(R^{N″}). In some other embodiments of any one of the aspects described herein, R²³ is —OCH₂CH₂—C(O)N(R^{N′})(R^{N″}).

In some embodiments, R²³ is hydrogen, hydroxyl, protected hydroxyl, halogen, optionally substituted C₁₋₃₀ alkyl, optionally substituted C₂₋₃₀alkenyl, optionally substituted C₂₋₃₀alkynyl, optionally substituted C₁₋₃₀ alkoxy (e.g., methoxy, 2-methoxyethoxy), alkoxyalkyl (e.g., 2-methoxyethyl), amino, alkylamino, dialkylamino, protected aminoalkyl, —O—C₄₋₃₀alkyl-ON(CH₂R₈)(CH₂R₉), —O—C₄₋₃₀alkyl-ON(CH₂R₈)(CH₂R₉), —O—N-methylacetamido, alkoxyoxycarboxylate, a solid support, a linker or a linker covalently attached to a solid support. For example, R²³ is hydrogen, hydroxyl, halogen, protected hydroxyl, optionally substituted C₁₋₃₀ alkyl, optionally substituted C₂₋₃₀alkenyl, optionally substituted C₂₋₃₀alkynyl, optionally substituted C₁₋₃₀alkoxy (e.g., methoxy), alkoxyalkyl (e.g., methoxyethyl), alkoxyalkylamine, alkoxyoxycarboxylate, amino, alkylamino, dialkylamino, protected aminoalkyl, —O—N-methylacetamido, —O—C₄₋₃₀alkyl-ON(CH₂R₈)(CH₂R₉), or —O—C₄₋₃₀alkyl-ON(CH₂R₈)(CH₂R₉).

In some embodiments of any one of the aspects described herein, R²³ is a bond to an internucleotide linkage to a subsequent nucleotide, a linker or a linker covalently attached to a solid support. For example, R²³ is a bond to an internucleotide linkage to a subsequent nucleotide.

In some embodiments, one of R²² and R²³ is —OCH₂CH₂—X′—R^{M′} or —O(CH₂)ₙ₁—C(O)N(R^{N′})(R^{N″}), and the other of R²² and R²³ is hydrogen, hydroxyl, protected hydroxyl, a bond to an internucleotide linkage to a subsequent nucleotide, a 3′-oligonucleotide capping group, a solid support, a linker or a linker covalently bonded a solid support. For example, R²² is —OCH₂CH₂—X′—R^{M′} or —O(CH₂)ₙ₁—C(O)N(R^{N′})(R^{N″}), and R²³ is hydrogen, hydroxyl, protected hydroxyl, a bond to an internucleotide linkage to a subsequent nucleotide, a 3′-oligonucleotide capping group, a solid support, a linker or a linker covalently bonded a solid support. In another non-limiting example, R²³ is —OCH₂CH₂—X′—R^{M′} or —O(CH₂)ₙ₁—C(O)N(R^{N′})(R^{N″}), and R²² is hydrogen, hydroxyl, protected hydroxyl, a bond to an internucleotide linkage to a subsequent nucleotide, a 3′-oligonucleotide capping group, a solid support, a linker or a linker covalently bonded a solid support.

In some embodiments, R²² is —O(CH₂)ₘ₁—X^{M′}—R^{M′} (e.g., —OCH₂CH₂—X′—R^{M′}) or —O(CH₂)ₙ₁—C(O)N(R^{N′})(R^{N″}), and R²³ is a bond to an internucleotide linkage to a subsequent nucleotide, a linker or a linker covalently bonded a solid support.

In some embodiments, R²³ is —O(CH₂)ₘ₁—X^{M′}—R^{M′} (e.g., —OCH₂CH₂—X′—R^{M′}) or —O(CH₂)ₙ₁—C(O)N(R^{N′})(R^{N″}), and R²² is a bond to an internucleotide linkage to a subsequent nucleotide, a linker or a linker covalently bonded a solid support.

### R²⁵

In nucleosides of Formula (II), R²⁵ represents —O(CH₂)ₘ₁—X^{M′}—R^{M′} (e.g., —OCH₂CH₂—X′—R^{M′}), —O(CH₂)ₙ₁—C(O)N(R^{N′})(R^{N″}), a bond to an internucleotide linkage to a preceding nucleotide, hydrogen, hydroxyl, protected hydroxyl, optionally substituted C₁₋₃₀ alkyl, optionally substituted C₂₋₃₀alkenyl, optionally substituted C₂₋₃₀alkynyl, optionally substituted C₁₋₃₀ alkoxy, optionally substituted 3-8 membered heterocyclyl (e.g., morpholin-1-yl, piperidin-1-yl, or pyrrolidin-1-yl), halogen, alkoxyalkyl (e.g., 2-methoxyethyl), alkoxyalkylamine, alkoxyoxycarboxylate, amino, alkylamino, dialkylamino, —O—C₄₋₃₀alkyl-ON(CH₂R⁸)(CH₂R⁹), —O—C₄₋₃₀alkyl-ON(CH₂R⁸)(CH₂R⁹), vinylphosphonate (VP) group (e.g., ═CH—X^{P}, X^{P} is a phosphate group), C₃₋₆ cycloalkylphosphonate (e.g., cyclopropylphosphonate), monophosphate ((HO)₂(O)P—O-5′), diphosphate ((HO)₂(O)P—O—P(HO)(O)—O-5′), triphosphate ((HO)₂(O)P—O—(HO)(O)P—O—P(HO)(O)—O-5′); monothiophosphate (phosphorothioate, (HO)₂(S)P—O-5′), monodithiophosphate (phosphorodithioate; (HO)(HS)(S)P—O-5′), phosphorothiolate ((HO)₂(O)P—S-5′); alpha-thiotriphosphate; beta-thiotriphosphate; gamma-thiotriphosphate; phosphoramidates ((HO)₂(O)P—NH-5′, (HO)(NH₂)(O)P—O-5′), alkylphosphonates [(R^{P})(OH)(O)P—O-5′, R^{P} is optionally substituted C₁₋₃₀ alkyl, e.g., methyl, ethyl, isopropyl, or propyl)], alkyletherphosphonates [(R^{P1})(OH)(O)P—O-5′, R^{P1} is alkoxyalkyl, e.g., methoxymethyl (CH₂OMe) or ethoxymethyl], (HO)₂(X)P—O[—(CH₂)ₐ—O—P(X)(OH)—O]_{b}-5′ or (HO)₂(X)P—O[—(CH₂)ₐ—P(X)(OH)—O]_{b}-5′ or (HO)₂(X)P—[—(CH₂)ₐ—O—P(X)(OH)—O]_{b}-5′, or optionally substituted alkyl, and dialkyl terminal phosphates and phosphate mimics (e.g., HO[—(CH₂)ₐ—O—P(X)(OH)—O]_{b}-5′, H2N[—(CH₂)ₐ—O—P(X)(OH)—O]_{b}-5′, H[—(CH₂)ₐ—O—P(X)(OH)—O]_{b}-5′, Me₂N[—(CH₂)ₐ—O—P(X)(OH)—O]_{b}-5′, HO[—(CH₂)ₐ—P(X)(OH)—O]_{b}-5′, H₂N[—(CH₂)ₐ—P(X)(OH)—O]_{b}-5′, H[—(CH₂)ₐ—P(X)(OH)—O]_{b}-5′, Me₂N[—(CH₂)ₐ—P(X)(OH)—O]_{b}-5′, wherein: X is O or S; a and b are each independently 1-10; and each R⁸ and R⁹ is independently H, a targeting ligand (e.g., GalNac), a pharmacokinetics modifier, optionally substituted C₁₋₃₀ alkyl, optionally substituted C₁₋₃₀ alkenyl, or optionally substituted C₁₋₃₀alkynyl.

In some embodiments of any one of the aspects, R²⁵ is —O(CH₂)ₘ₁—X′—R^{M′}. For example, R²⁵ is —O(CH₂)^{m1}—O—R^{M′}. In another non-limiting example, R²⁵ is —O(CH₂)ₘ₁—S—R^{M′}.

In some embodiments of any one of the aspects, R²⁵ is —OCH₂CH₂—X′—R^{M′}. For example, R²⁵ is —OCH₂CH₂—O—R^{M′}. In another non-limiting example, R²⁵ is —OCH₂CH₂—S—R^{M′}.

In some embodiments of any one the aspects described herein, R²⁵ is —O(CH₂)ₙ₁—C(O)N(R^{N′})(R^{N″}). It is noted, when R²⁵ is —O(CH₂)ₙ₁—C(O)N(R^{N′})(R^{N″}) then n1 can be 1 or 2. Accordingly, in some embodiments of any one the aspects described herein, R²⁵ is —OCH₂—C(O)N(R^{N′})(R^{N″}). In some other embodiments of any one of the aspects described herein, R²⁵ is —OCH₂CH₂—C(O)N(R^{N′})(R^{N″}).

In some nucleosides of Formula (II), R²⁵ is a bond to an internucleotide linkage to a preceding nucleotide, hydroxyl, protected hydroxyl, optionally substituted C₁₋₃₀ alkoxy, vinylphosphonate (VP) group, monophosphate, diphosphate, triphosphate, monothiophosphate (phosphorothioate), monodithiophosphate, phosphorothiolate, alpha-thiotriphosphate, beta-thiotriphosphate, gamma-thiotriphosphate, phosphoramidate, alkylphosphonate, alkyletherphosphonate, dialkyl terminal phosphate, phosphate mimic, or a bond to an internucleotide linkage to a preceding nucleotide. For example, R²⁵ is hydroxyl, optionally substituted C₁₋₃₀ alkoxy, vinylphosphonate (VP) group, monophosphate, diphosphate, triphosphate, monothiophosphate (phosphorothioate), monodithiophosphate, phosphorothiolate, alpha-thiotriphosphate, beta-thiotriphosphate, or gamma-thiotriphosphate.

In some embodiments of any one of the aspects described herein, R²⁵ is a bond to an internucleotide linkage to a preceding nucleotide, hydroxyl, protected hydroxyl, optionally substituted C₂₋₃₀alkenyl, optionally substituted C₁₋₃₀ alkoxy or a vinylphosphonate (VP) group.

In some embodiments of any one of the aspects described herein, R²⁵ is a bond to an internucleotide linkage to a preceding nucleotide.

In some embodiments of any one of the aspects described herein, R²⁵ is a hydroxyl or protected hydroxyl.

In some embodiments of any one of the aspects described herein, R²⁵ is optionally substituted C₂₋₃₀alkenyl or optionally substituted C₁₋₃₀ alkoxy.

In some embodiments of any one of the aspects described herein, R²⁵ is a vinylphosphonate group.

In some embodiments of any one of the aspects described herein, the methylene connecting the R²⁵ to the rest of the nucleoside of Formula (II) is absent and R²⁵ is connected directly to the rest of the nucleoside of Formula (II).

In some embodiments of any one of the aspects described herein, R²⁵ is —CH(R⁵¹)—X⁵—R⁵², where X⁵ is absent, a bond or O; R⁵¹ is hydrogen, optionally substituted C₁₋₃₀alkyl, optionally substituted —C₂₋₃₀alkenyl, or optionally substituted —C₂₋₃₀alkynyl, and R⁵² is a bond to an internucleoside linkage to the preceding nucleotide.

In some embodiments of any one of the aspects described herein, X⁵ is O or a bond. For example, X⁵ is O. In some other embodiments of any one of the aspects described herein, X⁵ is absent, i.e., R²⁵ is-CH(R⁵¹)R⁵²

In some embodiments of the any one of the aspects described herein, R²⁵ is —CH(R⁵¹)—R⁵² or —C(R⁵¹)═CHR⁵², where R⁵¹ is hydrogen, optionally substituted C₁₋₃₀alkyl, optionally substituted —C₂₋₃₀alkenyl, or optionally substituted —C₂₋₃₀alkynyl, and R⁵² is a bond to an internucleoside linkage to the preceding nucleotide.

In some embodiments of the any one of the aspects described herein, R²⁵ is —CH(R⁵¹)—X⁵—R⁵². For example, R²⁵ is —CH(R⁵¹)—X⁵—R⁵² and where R⁵¹ is H or C₁-C₃₀alkyl optionally substituted with 1, 2, 3, 4 or 5 substituents independently selected from OH, CN, SC(O)Ph, oxo (═O), SH, SO₂NH₂, SO₂(C₁-C₄)alkyl, SO₂NH(C₁-C₄)alkyl, halogen, carbonyl, thiol, cyano, NH₂, NH(C₁-C₄)alkyl, N[(C₁-C₄)alkyl]₂, C(O)NH₂, COOH, COOMe, acetyl, (C₁-C₈)alkyl, O(C₁-C₈)alkyl (i.e., C₁-C₈alkoxy), O(C₁-C₈)haloalkyl, (C₂-C₈)alkenyl, (C₂-C₈)alkynyl, haloalkyl, thioalkyl, cyanomethylene, alkylaminyl, aryl, heteroaryl, substituted aryl, NH₂—C(O)-alkylene, NH(Me)-C(O)-alkylene, CH₂—C(O)-alkyl, C(O)-alkyl, alkylcarbonylaminyl, CH₂—[CH(OH)]ₘ—(CH₂)ₚ—OH, CH₂—[CH(OH)]ₘ—(CH₂)ₚ—NH₂ or CH₂-aryl-alkoxy, where “m” and “p” are independently 1, 2, 3, 4, 5 or 6. For example, R⁵¹ is H. In some other non-limiting examples, R⁵¹ is C₁-C₃₀alkyl optionally substituted with a NH₂, OH, C(O)NH₂, COOH, halo, SH, or C₁-C₆alkoxy.

In some embodiments of the any one of the aspects described herein, R²⁵ is —CH(R⁵¹)—O—R⁵², where R⁵¹ is H or C₁-C₃₀alkyl optionally substituted with 1, 2, 3, 4 or 5 substituents independently selected from 1, 2, 3, 4 or 5 substituents independently selected from OH, CN, SC(O)Ph, oxo (═O), SH, SO₂NH₂, SO₂(C₁-C₄)alkyl, SO₂NH(C₁-C₄)alkyl, halogen, carbonyl, thiol, cyano, NH₂, NH(C₁-C₄)alkyl, N[(C₁-C₄)alkyl]₂, C(O)NH₂, COOH, COOMe, acetyl, (C₁-C₈)alkyl, O(C₁-C₈)alkyl (i.e., C₁-C₈alkoxy), O(C₁-C₈)haloalkyl, (C₂-C₈)alkenyl, (C₂-C₈)alkynyl, haloalkyl, thioalkyl, cyanomethylene, alkylaminyl, aryl, heteroaryl, substituted aryl, NH₂—C(O)-alkylene, NH(Me)-C(O)-alkylene, CH₂—C(O)-alkyl, C(O)-alkyl, alkylcarbonylaminyl, CH₂—[CH(OH)]ₘ—(CH₂)ₚ—OH, CH₂—[CH(OH)]ₘ—(CH₂)ₚ—NH₂ or CH₂-aryl-alkoxy, where “m” and “p” are independently 1, 2, 3, 4, 5 or 6. For example, R⁵¹ is H. In some other non-limiting examples, R⁵¹ is C₁-C₃₀alkyl optionally substituted with a NH₂, OH, C(O)NH₂, COOH, halo, SH, or C₁-C₆alkoxy.

In some embodiments of any one of the aspects described herein, R²⁵ is —C(R⁵¹)═CHR⁵². It is noted that the double bond in —C(R⁵¹)═CHR⁵² can be in the cis or trans configuration. Accordingly, in some embodiments of any one of the aspects, R²⁵ is —C(R⁵¹)═CHR⁵² and wherein the double bond is in the cis configuration. In some other embodiments of any one of the aspects, R²⁵ is —C(R⁵¹)═CHR⁵² and wherein the double bond is in the trans configuration. In some embodiments of any one of the aspects described herein, R²⁵ is —CH═CHR⁵².

In some embodiments of any one of the aspects described herein, R⁵² is a bond to an internucleoside linkage to the preceding nucleotide.

In embodiments of the any one of the aspects described herein, R²⁵ is optionally substituted C₁₋₆alkyl-R⁵³, optionally substituted —C₂₋₆alkenyl-R⁵³, or optionally substituted —C₂₋₆alkynyl-R⁵³. In embodiments of the any one of the aspects described herein, R⁵³ can be —OR⁵⁴, —SR⁵⁵, —P(O)(OR⁵⁶)₂, —P(S)(OR⁵⁶)₂, —P(S)(SR⁵⁷)(OR⁵⁶), —P(S)(SR⁵⁷)₂, —OP(O)(OR⁵⁶)₂, —OP(S)(OR⁵⁶)₂, —OP(S)(SR⁵⁷)(OR⁵⁶), —OP(S)(SR⁵⁷)₂, —SP(O)(OR⁵⁶)₂, —SP(S)(OR⁵⁶)₂, —SP(S)(SR⁵⁷)(OR⁵⁶), or —SP(S)(SR⁵⁷)₂; where R⁵⁴ is hydrogen or oxygen protecting group; R⁵⁵ is hydrogen or sulfur protecting group; each R⁵⁶ is independently hydrogen, optionally substituted C₁₋₃₀alkyl, optionally substituted C₂₋₃₀alkenyl, or optionally substituted C₂₋₃₀alkynyl, or an oxygen-protecting group; and each R⁵⁷ is independently hydrogen, optionally substituted C₁₋₃₀alkyl, optionally substituted C₂₋₃₀alkenyl, or optionally substituted C₂₋₃₀alkynyl, or a sulfur-protecting group.

In some embodiments of any one of the aspects, at least one R⁵⁶ in —P(O)(OR⁵⁶)₂, —P(S)(OR⁵⁶)₂, —P(S)(SR⁵⁷)(OR⁵⁶), —OP(O)(OR⁵⁶)₂, —OP(S)(OR⁵⁶)₂, —OP(S)(SR⁵⁷)(OR⁵⁶), SP(O)(OR⁵⁶)₂, —SP(S)(OR⁵⁶)₂, and —SP(S)(SR⁵⁷)(OR⁵⁶) is hydrogen.

In some other embodiments of any one of the aspects, at least one R⁵⁶ in —P(O)(OR⁵⁶)₂, —P(S)(OR⁵⁶)₂, —P(S)(SR⁵⁷)(OR⁵⁶), —OP(O)(OR⁵⁶)₂, —OP(S)(OR⁵⁶)₂, —OP(S)(SR⁵⁷)(OR⁵⁶), SP(O)(OR⁵⁶)₂, —SP(S)(OR⁵⁶)₂, or —SP(S)(SR⁵⁷)(OR⁵⁶) is not hydrogen. For example, at least one at least one R⁵⁶ in P(O)(OR⁵⁶)₂, —P(S)(OR⁵⁶)₂, —P(S)(SR⁵⁷)(OR⁵⁶), —OP(O)(OR⁵⁶)₂, —OP(S)(OR⁵⁶)₂, —OP(S)(SR⁵⁷)(OR⁵⁶), SP(O)(OR⁵⁶)₂, —SP(S)(OR⁵⁶)₂, and —SP(S)(SR⁵⁷)(OR⁵⁶) is optionally substituted C₁₋₃₀alkyl, optionally substituted C₂₋₃₀alkenyl, or optionally substituted C₂₋₃₀alkynyl, or an oxygen-protecting group.

In some embodiments of any one of the aspects, at least one R⁵⁶ is H and at least one R⁵⁶ is other than H in —P(O)(OR⁵⁶)₂, —P(S)(OR⁵⁶)₂, —P(S)(SR⁵⁷)(OR⁵⁶), —OP(O)(OR⁵⁶)₂, —OP(S)(OR⁵⁶)₂, —OP(S)(SR⁵⁷)(OR⁵⁶), SP(O)(OR⁵⁶)₂, —SP(S)(OR⁵⁶)₂, and —SP(S)(SR⁵⁷)(OR⁵⁶).

In some embodiments of any one of the aspects, all R⁵⁶ are H in —P(O)(OR⁵⁶)₂, —P(S)(OR⁵⁶)₂, —P(S)(SR⁵⁷)(OR⁵⁶), —OP(O)(OR⁵⁶)₂, —OP(S)(OR⁵⁶)₂, —OP(S)(SR⁵⁷)(OR⁵⁶), —OP(S)(SR⁵⁷)₂, —SP(O)(OR⁵⁶)₂, —SP(S)(OR⁵⁶)₂, —SP(S)(SR⁵⁷)(OR⁵⁶), and —SP(S)(SR⁵⁷)₂.

In some embodiments of any one of the aspects, all R⁵⁶ are other than H in in —P(O)(OR⁵⁶)₂, —P(S)(OR⁵⁶)₂, —P(S)(SR⁵⁷)(OR⁵⁶), —OP(O)(OR⁵⁶)₂, —OP(S)(OR⁵⁶)₂, —OP(S)(SR⁵⁷)(OR⁵⁶), —OP(S)(SR⁵⁷)₂, —SP(O)(OR⁵⁶)₂, —SP(S)(OR⁵⁶)₂, —SP(S)(SR⁵⁷)(OR⁵⁶), and —SP(S)(SR⁵⁷)₂.

In some embodiments of any one of the aspects, at least one R⁵⁷ in —P(S)(SR⁵⁷)(OR⁵⁶)—P(S)(SR⁵⁷)₂, —OP(S)(OR⁵⁶)₂, —OP(S)(SR⁵⁷)(OR⁵⁶), —OP(S)(SR⁵⁷)₂, —SP(S)(SR⁵⁷)(OR⁵⁶), and —SP(S)(SR⁵⁷)₂ is H.

In some embodiments of any one of the aspects, at least one R⁵⁷ in —P(S)(SR⁵⁷)(OR⁵⁶)—P(S)(SR⁵⁷)₂, —OP(S)(OR⁵⁶)₂, —OP(S)(SR⁵⁷)(OR⁵⁶), —OP(S)(SR⁵⁷)₂, —SP(S)(SR⁵⁷)(OR⁵⁶), and —SP(S)(SR⁵⁷)₂ is other than H. For example, at least one R⁵⁷ in —P(S)(SR⁵⁷)(OR⁵⁶), —P(S)(SR⁵⁷)₂, —OP(S)(OR⁵⁶)₂, —OP(S)(SR⁵⁷)(OR⁵⁶), —OP(S)(SR⁵⁷)₂, —SP(S)(SR⁵⁷)(OR⁵⁶), and —SP(S)(SR⁵⁷)₂ is optionally substituted C₁₋₃₀alkyl, optionally substituted C₂₋₃₀alkenyl, or optionally substituted C₂₋₃₀alkynyl, or an sulfur-protecting group.

In some embodiments of any one of the aspects, at least one R⁵⁷ is H and at least one R⁵⁷ is other than H in —P(S)(SR⁵⁷)₂, —OP(S)(SR⁵⁷)₂ and —SP(S)(SR⁵⁷)₂.

In some embodiments, all R⁵⁷ are H in —P(S)(SR⁵⁷)(OR⁵⁶), —P(S)(SR⁵⁷)₂, —OP(S)(OR⁵⁶)₂, —OP(S)(SR⁵⁷)(OR⁵⁶), —OP(S)(SR⁵⁷)₂, —SP(S)(SR⁵⁷)(OR⁵⁶), and —SP(S)(SR⁵⁷)₂.

In some embodiments, all R⁵⁷ are other than H in —P(S)(SR⁵⁷)(OR⁵⁶), —P(S)(SR⁵⁷)₂, —OP(S)(OR⁵⁶)₂, —OP(S)(SR⁵⁷)(OR⁵⁶), —OP(S)(SR⁵⁷)₂, —SP(S)(SR⁵⁷)(OR⁵⁶), and —SP(S)(SR⁵⁷)₂.

In some embodiments of any one of the aspects described herein, R²⁵ is optionally substituted —C₂₋₆alkenyl-R⁵³. For example, R²⁵ is —C₂₋₆alkenyl-R⁵³, where C₂₋₆alkenyl is optionally substituted with 1, 2, 3, 4 or 5 substituents independently selected from OH, CN, SC(O)Ph, oxo (═O), SH, SO₂NH₂, SO₂(C₁-C₄)alkyl, SO₂NH(C₁-C₄)alkyl, halogen, carbonyl, thiol, cyano, NH₂, NH(C₁-C₄)alkyl, N[(C₁-C₄)alkyl]₂, C(O)NH₂, COOH, COOMe, acetyl, (C₁-C₈)alkyl, O(C₁-C₈)alkyl (i.e., C₁-C₈alkoxy), O(C₁-C₈)haloalkyl, (C₂-C₈)alkenyl, (C₂-C₈)alkynyl, haloalkyl, thioalkyl, cyanomethylene, alkylaminyl, aryl, heteroaryl, substituted aryl, NH₂—C(O)-alkylene, NH(Me)-C(O)-alkylene, CH₂—C(O)-alkyl, C(O)-alkyl, alkylcarbonylaminyl, CH₂—[CH(OH)]ₘ—(CH₂)ₚ—OH, CH₂—[CH(OH)]ₘ—(CH₂)ₚ—NH₂ or CH₂-aryl-alkoxy, where “m” and “p” are independently 1, 2, 3, 4, 5 or 6; and R⁵³ is —P(O)(OR⁵⁶)₂, —P(S)(OR⁵⁶)₂, —P(S)(SR⁵⁷)(OR⁵⁶), —P(S)(SR⁵⁷)₂, —OP(O)(OR⁵⁶)₂, —OP(S)(OR⁵⁶)₂, —OP(S)(SR⁵⁷)(OR⁵⁶), —OP(S)(SR⁵⁷)₂, —SP(O)(OR⁵⁶)₂, —SP(S)(OR⁵⁶)₂, —SP(S)(SR⁵⁷)(OR⁵⁶), or —SP(S)(SR⁵⁷)₂.

In some embodiments of any one of the aspects, R²⁵ is —CH═CHR⁵³. It is noted that a double bond in the optionally substituted —C₂₋₆alkenyl-R⁵³ can be in the cis or trans configuration. Accordingly, in some embodiments of any one of the aspects, R²⁵ is —CH═CHR⁵³ and wherein the double bond is in the cis configuration. In some other embodiments of any one of the aspects, R²⁵ is —CH═CHR⁵³ and wherein the double bond is in the trans configuration.

In some embodiments of any one of the aspects, R²⁵ is —CH═CH—P(O)(OR⁵⁶)₂, —CH═CH—P(S)(OR⁵⁶)₂, —CH═CH—P(S)(SR⁵⁷)(OR⁵⁶), —CH═CH—P(S)(SR⁵⁷)₂, —CH═CH—OP(O)(OR⁵⁶)₂, —CH═CH—OP(S)(OR⁵⁶)₂, —CH═CH—OP(S)(SR⁵⁷)(OR⁵⁶), —CH═CH—OP(S)(SR⁵⁷)₂, —CH═CH—SP(O)(OR⁵⁶)₂, —CH═CH—SP(S)(OR⁵⁶)₂, —CH═CH—SP(S)(SR⁵⁷)(OR⁵⁶), or —CH═CH—SP(S)(SR⁵⁷)₂. For example, R²⁵ is —CH═CH—P(O)(OR⁵⁶)₂.

In some embodiments, of any one of the aspects, R⁵⁴ is hydrogen or an oxygen protecting group. For example, R⁵⁴ is hydrogen or 4,4′-dimethoxytrityl (DMT). In some preferred embodiments, R⁵⁴ is H.

In some embodiments of any one of the aspects described herein, R²⁵ is optionally substituted —C₁₋₆alkenyl-R⁵³. For example, R²⁵ is —C₁₋₆alkenyl-R⁵³, where C₁₋₆alkenyl is optionally substituted with 1, 2, 3, 4 or 5 substituents independently selected from OH, CN, SC(O)Ph, oxo (═O), SH, SO₂NH₂, SO₂(C₁-C₄)alkyl, SO₂NH(C₁-C₄)alkyl, halogen, carbonyl, thiol, cyano, NH₂, NH(C₁-C₄)alkyl, N[(C₁-C₄)alkyl]₂, C(O)NH₂, COOH, COOMe, acetyl, (C₁-C₈)alkyl, O(C₁-C₈)alkyl (i.e., C₁-C₈alkoxy), O(C₁-C₈)haloalkyl, (C₂-C₈)alkenyl, (C₂-C₈)alkynyl, haloalkyl, thioalkyl, cyanomethylene, alkylaminyl, aryl, heteroaryl, substituted aryl, NH₂—C(O)-alkylene, NH(Me)-C(O)-alkylene, CH₂—C(O)-alkyl, C(O)-alkyl, alkylcarbonylaminyl, CH₂—[CH(OH)]ₘ—(CH₂)ₚ—OH, CH₂—[CH(OH)]ₘ—(CH₂)ₚ—NH₂ or CH₂-aryl-alkoxy, where “m” and “p” are independently 1, 2, 3, 4, 5 or 6; and R⁵³ is —OR⁵⁴, —SR⁵⁵, —P(O)(OR⁵⁶)₂, —P(S)(OR⁵⁶)₂, —P(S)(SR⁵⁷)(OR⁵⁶), —P(S)(SR⁵⁷)₂, —OP(O)(OR⁵⁶)₂, —OP(S)(OR⁵⁶)₂, —OP(S)(SR⁵⁷)(OR⁵⁶), —OP(S)(SR⁵⁷)₂, —SP(O)(OR⁵⁶)₂, —SP(S)(OR⁵⁶)₂, —SP(S)(SR⁵⁷)(OR⁵⁶), or —SP(S)(SR⁵⁷)₂.

In some embodiments of any one of the aspects described herein, R²⁵ can be —CH(R⁵⁸)—R⁵³, where R⁵³ is —OR⁵⁴, —SR⁵⁵, —P(O)(OR⁵⁶)₂, —P(S)(OR⁵⁶)₂, —P(S)(SR⁵⁷)(OR⁵⁶), —P(S)(SR⁵⁷)₂, —OP(O)(OR⁵⁶)₂, —OP(S)(OR⁵⁶)₂, —OP(S)(SR⁵⁷)(OR⁵⁶), —OP(S)(SR⁵⁷)₂, —SP(O)(OR⁵⁶)₂, —SP(S)(OR⁵⁶)₂, —SP(S)(SR⁵⁷)(OR⁵⁶), or —SP(S)(SR⁵⁷)₂; and R⁵⁸ is H, optionally substituted C₁₋₃₀alkyl, optionally substituted C₂₋₃₀alkenyl, or optionally substituted C₂₋₃₀alkynyl.

In some embodiments of any one of the aspects described herein, R⁵⁸ is H or C₁-C30alkyl optionally substituted with 1, 2, 3, 4 or 5 substituents independently selected from OH, CN, SC(O)Ph, oxo (═O), SH, SO₂NH₂, SO₂(C₁-C₄)alkyl, SO₂NH(C₁-C₄)alkyl, halogen, carbonyl, thiol, cyano, NH₂, NH(C₁-C₄)alkyl, N[(C₁-C₄)alkyl]₂, C(O)NH₂, COOH, COOMe, acetyl, (C₁-C₈)alkyl, O(C₁-C₈)alkyl (i.e., C₁-C₈alkoxy), O(C₁-C₈)haloalkyl, (C₂-C₈)alkenyl, (C₂-C₈)alkynyl, haloalkyl, thioalkyl, cyanomethylene, alkylaminyl, aryl, heteroaryl, substituted aryl, NH₂—C(O)-alkylene, NH(Me)-C(O)-alkylene, CH₂—C(O)-alkyl, C(O)-alkyl, alkylcarbonylaminyl, CH₂—[CH(OH)]ₘ—(CH₂)ₚ—OH, CH₂—[CH(OH)]ₘ—(CH₂)ₚ—NH₂ or CH₂-aryl-alkoxy, where “m” and “p” are independently 1, 2, 3, 4, 5 or 6. In one non-limiting example, R⁵⁸ is H. In some other non-limiting examples, R⁵¹ is C₁-C₃₀alkyl optionally substituted with a substituent selected from NH₂, OH, C(O)NH₂, COOH, halo, SH, and C₁-C₆alkoxy.

In some embodiments of any one of the aspects described herein, R²⁵ is —CH(R⁵⁸)—O—R⁵⁹, where R⁵⁹ is H, —P(O)(OR⁵⁶)₂, —P(S)(OR⁵⁶)₂, —P(S)(SR⁵⁷)(OR⁵⁶), —P(S)(SR⁵⁷)₂, —OP(O)(OR⁵⁶)₂. For example, R²⁵ is —CH(R⁵⁸)—O—R⁵⁹, where R⁵⁸ is H or optionally substituted C₁-C₃₀alkyl and R⁵⁹ is H or —P(O)(OR⁵⁶)₂.

In some embodiments of any one of the aspects described herein, R²⁵ is —CH(R⁵⁸)—S—R⁶⁰, where R⁶⁰ is H, —P(O)(OR⁵⁶)₂, —P(S)(OR⁵⁶)₂, —P(S)(SR⁵⁷)(OR⁵⁶), —P(S)(SR⁵⁷)₂, —OP(O)(OR⁵⁶)₂.

It is noted that in nucleosides of Formula (II) no more than one of R²² and R²³ is a bond to an internucleotide linkage to a subsequent nucleotide, and when both of R²² and R²³ are not a bond to an internucleotide linkage, then R²⁵ is a bond to an internucleotide linkage to a preceding nucleotide.

In some embodiments, one of R²² and R²³ is —O(CH₂)ₘ₁—X^{M′}—R^{M′} (e.g., —OCH₂CH₂—X′—R^{M′}) or —O(CH₂)ₙ₁—C(O)N(R^{N′})(R^{N″}), the other of R²² and R²³ is hydrogen, hydroxyl, protected hydroxyl, a bond to an internucleotide linkage to a subsequent nucleotide, a 3′-oligonucleotide capping group, a solid support, a linker or a linker covalently bonded a solid support, and R²⁵ is hydroxyl, protected hydroxyl, a bond to an internucleotide linkage to a preceding nucleotide or a vinyl phosphate group. For example, R²² is —O(CH₂)ₘ₁—X^{M′}—R^{M′} (e.g., —OCH₂CH₂—X′—R^{M′}) or —O(CH₂)ₙ₁—C(O)N(R^{N′})(R^{N″}), R²³ is hydrogen, hydroxyl, protected hydroxyl, a bond to an internucleotide linkage to a subsequent nucleotide, a 3′-oligonucleotide capping group, a solid support, a linker or a linker covalently bonded a solid support, and R²⁵ is hydroxyl, protected hydroxyl, a bond to an internucleotide linkage to a preceding nucleotide or a vinyl phosphate group. In another non-limiting example, R²³ is —OCH₂CH₂—X′—R^{M′} or —O(CH₂)ₙ₁—C(O)N(R^{N′})(R^{N″}), R²² is hydrogen, hydroxyl, protected hydroxyl, a bond to an internucleotide linkage to a subsequent nucleotide, a 3′-oligonucleotide capping group, a solid support, a linker or a linker covalently bonded a solid support, and R²⁵ is hydroxyl, protected hydroxyl, a bond to an internucleotide linkage to a preceding nucleotide or a vinyl phosphate group.

In some embodiments, R²² is —O(CH₂)ₘ₁—X^{M′}—R^{M′} (e.g., —OCH₂CH₂—X′—R^{M′}) or —O(CH₂)ₙ₁—C(O)N(R^{N′})(R^{N″}), R²³ is a bond to an internucleotide linkage to a subsequent nucleotide, a linker or a linker covalently bonded a solid support, and R²⁵ is hydroxyl, protected hydroxyl, a bond to an internucleotide linkage to a preceding nucleotide or a vinyl phosphate group.

In some embodiments, R²³ is —O(CH₂)ₘ₁—X^{M′}—R^{M′} (e.g., —OCH₂CH₂—X′—R^{M′}) or —O(CH₂)ₙ₁—C(O)N(R^{N′})(R^{N″}), R²² is a bond to an internucleotide linkage to a subsequent nucleotide, a linker or a linker covalently bonded a solid support, and R²⁵ is hydroxyl, protected hydroxyl, a bond to an internucleotide linkage to a preceding nucleotide or a vinyl phosphate group.

### R^{22′}

In nucleosides of Formula (II′), R^{22′} is —O(CH₂)ₙ₁—C(O)OR^{LV}, hydrogen, hydroxyl, protected hydroxyl, halogen, optionally substituted C₁₋₃₀ alkyl, optionally substituted C₂₋₃₀alkenyl, optionally substituted C₂₋₃₀alkynyl, optionally substituted C₁₋₃₀alkoxy (e.g., methoxy, 2-methoxyethoxy), alkoxyalkyl (e.g., 2-methoxyethyl), alkoxyalkylamine, alkoxyoxycarboxylate, amino, alkylamino, dialkylamino, protected aminoalkyl, 5-8 membered heterocyclyl, —O—C₄₋₃₀alkyl-ON(CH₂R⁸)(CH₂R⁹), —O—N-methylacetamido, —O—C₄₋₃₀alkyl-ON(CH₂R⁸)(CH₂R⁹), a bond to an internucleotide linkage to a subsequent nucleotide, a 3′-oligonucleotide capping group, a ligand, a linker covalently bonded to one or more ligands, a solid support, a linker or a linker covalently bonded a solid support.

In some embodiments of any one the aspects described herein, R^{22′} is —O(CH₂)ₙ₁—C(O)OR^{LV}. It is noted, when R^{22′} is —O(CH₂)ₙ₁—C(O)OR^{LV}, then n1 can be 1 or 2. Accordingly, in some embodiments of any one the aspects described herein, R^{22′} is —OCH₂—C(O)OR^{LV}. In some other embodiments of any one of the aspects described herein, R^{22′} is —OCH₂CH₂—C(O)OR^{LV}

In some embodiments, R^{22′} is hydrogen, hydroxyl, protected hydroxyl, halogen, optionally substituted C₁₋₃₀ alkyl, optionally substituted C₂₋₃₀alkenyl, optionally substituted C₂₋₃₀alkynyl, optionally substituted C₁₋₃₀ alkoxy (e.g., methoxy, 2-methoxyethoxy), alkoxyalkyl (e.g., 2-methoxyethyl), amino, alkylamino, dialkylamino, protected aminoalkyl, —O—C₄₋₃₀alkyl-ON(CH₂R₈)(CH₂R₉), —O—C₄₋₃₀alkyl-ON(CH₂R₈)(CH₂R₉), —O—N-methylacetamido, alkoxyoxycarboxylate, a solid support, a linker or a linker covalently attached to a solid support. For example, R^{22′} is hydrogen, hydroxyl, halogen, protected hydroxyl, optionally substituted C₁₋₃₀ alkyl, optionally substituted C₂₋₃₀alkenyl, optionally substituted C₂₋₃₀alkynyl, optionally substituted C₁₋₃₀alkoxy (e.g., methoxy), alkoxyalkyl (e.g., methoxyethyl), alkoxyalkylamine, alkoxyoxycarboxylate, amino, alkylamino, dialkylamino, protected aminoalkyl, —O—N-methylacetamido, —O—C₄₋₃₀alkyl-ON(CH₂R₈)(CH₂R₉), or —O—C₄₋₃₀alkyl-ON(CH₂R₈)(CH₂R₉).

In some embodiments of any one of the aspects described herein, R^{22′} is a bond to an internucleotide linkage to a subsequent nucleotide, a linker or a linker covalently attached to a solid support. For example, R^{22′} is a bond to an internucleotide linkage to a subsequent nucleotide.

### R^{23′}

In nucleosides of Formula (II′), R^{23′} is —O(CH₂)ₙ₁—C(O)OR^{LV}, hydrogen, hydroxyl, protected hydroxyl, halogen, optionally substituted C₁₋₃₀ alkyl, optionally substituted C₂₋₃₀alkenyl, optionally substituted C₂₋₃₀alkynyl, optionally substituted C₁₋₃₀ alkoxy (e.g., methoxy, 2-methoxyethoxy), alkoxyalkyl (e.g., 2-methoxyethyl), alkoxyalkylamine, alkoxyoxycarboxylate, amino, alkylamino, dialkylamino, protected aminoalkyl, 5-8 membered heterocyclyl, —O—C₄₋₃₀alkyl-ON(CH₂R⁸)(CH₂R⁹), —O—N-methylacetamido, —O—C₄₋₃₀alkyl-ON(CH₂R⁸)(CH₂R⁹), a bond to an internucleotide linkage to a subsequent nucleotide, a 3′-oligonucleotide capping group, a ligand, a linker covalently bonded to one or more ligands, a solid support, a linker or a linker covalently bonded a solid support.

In some embodiments of any one the aspects described herein, R^{23′} is —O(CH₂)ₙ₁—C(O)OR^{LV}. It is noted, when R^{23′} is —O(CH₂)ₙ₁—C(O)OR^{LV}, then n1 can be 1 or 2. Accordingly, in some embodiments of any one the aspects described herein, R^{23′} is —OCH₂—C(O)OR^{LV}. In some other embodiments of any one of the aspects described herein, R^{23′} is —OCH₂CH₂—C(O)OR^{LV}.

In some embodiments, R^{23′} is hydrogen, hydroxyl, protected hydroxyl, halogen, optionally substituted C₁₋₃₀ alkyl, optionally substituted C₂₋₃₀alkenyl, optionally substituted C₂₋₃₀alkynyl, optionally substituted C₁₋₃₀ alkoxy (e.g., methoxy, 2-methoxyethoxy), alkoxyalkyl (e.g., 2-methoxyethyl), amino, alkylamino, dialkylamino, protected aminoalkyl, —O—C₄₋₃₀alkyl-ON(CH₂R₈)(CH₂R₉), —O—C₄₋₃₀alkyl-ON(CH₂R₈)(CH₂R₉), —O—N-methylacetamido, alkoxyoxycarboxylate, a solid support, a linker or a linker covalently attached to a solid support. For example, R^{23′} is hydrogen, hydroxyl, halogen, protected hydroxyl, optionally substituted C₁₋₃₀ alkyl, optionally substituted C₂₋₃₀alkenyl, optionally substituted C₂₋₃₀alkynyl, optionally substituted C₁₋₃₀alkoxy (e.g., methoxy), alkoxyalkyl (e.g., methoxyethyl), alkoxyalkylamine, alkoxyoxycarboxylate, amino, alkylamino, dialkylamino, protected aminoalkyl, —O—N-methylacetamido, —O—C₄₋₃₀alkyl-ON(CH₂R₈)(CH₂R₉), or —O—C₄₋₃₀alkyl-ON(CH₂R₈)(CH₂R₉).

In some embodiments of any one of the aspects described herein, R^{23′} is a bond to an internucleotide linkage to a subsequent nucleotide, a linker or a linker covalently attached to a solid support. For example, R^{23′} is a bond to an internucleotide linkage to a subsequent nucleotide.

In some embodiments, one of R^{22′} and R^{23′} is —O(CH₂)ₙ₁—C(O)OR^{LV}, and the other of R^{22′} and R^{23′} is hydrogen, hydroxyl, protected hydroxyl, a bond to an internucleotide linkage to a subsequent nucleotide, a 3′-oligonucleotide capping group, a solid support, a linker or a linker covalently bonded a solid support. For example, R^{22′} is —O(CH₂)ₙ₁—C(O)OR^{LV}, and R^{23′} is hydrogen, hydroxyl, protected hydroxyl, a bond to an internucleotide linkage to a subsequent nucleotide, a 3′-oligonucleotide capping group, a solid support, a linker or a linker covalently bonded a solid support. In another non-limiting example, R^{23′} is —O(CH₂)ₙ₁—C(O)OR^{LV}, and R^{22′} is hydrogen, hydroxyl, protected hydroxyl, a bond to an internucleotide linkage to a subsequent nucleotide, a 3′-oligonucleotide capping group, a solid support, a linker or a linker covalently bonded a solid support.

In some embodiments, R^{22′} is —O(CH₂)ₙ₁—C(O)OR^{LV}, and R^{23′} is a bond to an internucleotide linkage to a subsequent nucleotide, a linker or a linker covalently bonded a solid support.

In some embodiments, R^{23′} is —O(CH₂)ₙ₁—C(O)OR^{LV}, and R^{22′} is a bond to an internucleotide linkage to a subsequent nucleotide, a linker or a linker covalently bonded a solid support.

### R^{25′}

In nucleosides of Formula (II′), R^{25′} represents —OCH₂CH₂—X′—R^{M′}, —O(CH₂)ₙ₁—C(O)N(R^{N′})(R^{N″}), a bond to an internucleotide linkage to a preceding nucleotide, hydrogen, hydroxyl, protected hydroxyl, optionally substituted C₁₋₃₀ alkyl, optionally substituted C₂₋₃₀alkenyl, optionally substituted C₂₋₃₀alkynyl, optionally substituted C₁₋₃₀ alkoxy, optionally substituted 3-8 membered heterocyclyl (e.g., morpholin-1-yl, piperidin-1-yl, or pyrrolidin-1-yl), halogen, alkoxyalkyl (e.g., 2-methoxyethyl), alkoxyalkylamine, alkoxyoxycarboxylate, amino, alkylamino, dialkylamino, —O—C₄₋₃₀alkyl-ON(CH₂R⁸)(CH₂R⁹), —O—C₄₋₃₀alkyl-ON(CH₂R⁸)(CH₂R⁹), vinylphosphonate (VP) group (e.g., ═CH—X^{P}, X^{P} is a phosphate group), C₃₋₆ cycloalkylphosphonate (e.g., cyclopropylphosphonate), monophosphate ((HO)₂(O)P—O-5′), diphosphate ((HO)₂(O)P—O—P(HO)(O)—O-5′), triphosphate ((HO)₂(O)P—O—(HO)(O)P—O—P(HO)(O)—O-5′); monothiophosphate (phosphorothioate, (HO)₂(S)P—O-5′), monodithiophosphate (phosphorodithioate; (HO)(HS)(S)P—O-5′), phosphorothiolate ((HO)₂(O)P—S-5′); alpha-thiotriphosphate; beta-thiotriphosphate; gamma-thiotriphosphate; phosphoramidates ((HO)₂(O)P—NH-5′, (HO)(NH₂)(O)P—O-5′), alkylphosphonates [(R^{P})(OH)(O)P—O-5′, R^{P} is optionally substituted C₁₋₃₀ alkyl, e.g., methyl, ethyl, isopropyl, or propyl)], alkyletherphosphonates [(R^{P})(OH)(O)P—O-5′, R¹ is alkoxyalkyl, e.g., methoxymethyl (CH₂OMe) or ethoxymethyl], (HO)₂(X)P—O[—(CH₂)ₐ—O—P(X)(OH)—O]_{b}-5′ or (HO)₂(X)P—O[—(CH₂)ₐ—P(X)(OH)—O]_{b}-5′ or (HO)₂(X)P—[—(CH₂)ₐ—O—P(X)(OH)—O]_{b}-5′, or optionally substituted alkyl, and dialkyl terminal phosphates and phosphate mimics (e.g., HO[—(CH₂)ₐ—O—P(X)(OH)—O]_{b}-5′, H2N[—(CH₂)ₐ—O—P(X)(OH)—O]_{b}-5′, H[—(CH₂)ₐ—O—P(X)(OH)—O]_{b}-5′, Me₂N[—(CH₂)ₐ—O—P(X)(OH)—O]_{b}-5′, HO[—(CH₂)ₐ—P(X)(OH)—O]_{b}-5′, H2N[—(CH₂)ₐ—P(X)(OH)—O]_{b}-5′, H[—(CH₂)ₐ—P(X)(OH)—O]_{b}-5′, Me₂N[—(CH₂)ₐ—P(X)(OH)—O]_{b}-5′, wherein: X is O or S; a and b are each independently 1-10; and each R⁸ and R⁹ is independently H, a targeting ligand (e.g., GalNac), a pharmacokinetics modifier, optionally substituted C₁₋₃₀ alkyl, optionally substituted C₁₋₃₀ alkenyl, or optionally substituted C₁₋₃₀alkynyl.

In some embodiments of any one the aspects described herein, R^{25′} is —O(CH₂)ₙ₁—C(O)OR^{LV}. It is noted, when R^{25′} is —O(CH₂)ₙ₁—C(O)OR^{LV}, then n1 can be 1 or 2. Accordingly, in some embodiments of any one the aspects described herein, R^{25′} is —OCH₂—C(O)OR^{LV}. In some other embodiments of any one of the aspects described herein, R^{25′} is —OCH₂CH₂—C(O)OR^{LV}.

In some nucleosides of Formula (II′), R^{25′} is a bond to an internucleotide linkage to a preceding nucleotide, hydroxyl, protected hydroxyl, optionally substituted C₁₋₃₀ alkoxy, vinylphosphonate (VP) group, monophosphate, diphosphate, triphosphate, monothiophosphate (phosphorothioate), monodithiophosphate, phosphorothiolate, alpha-thiotriphosphate, beta-thiotriphosphate, gamma-thiotriphosphate, phosphoramidate, alkylphosphonate, alkyletherphosphonate, dialkyl terminal phosphate, phosphate mimic, or a bond to an internucleotide linkage to a preceding nucleotide. For example, R^{25′} is hydroxyl, optionally substituted C₁₋₃₀ alkoxy, vinylphosphonate (VP) group, monophosphate, diphosphate, triphosphate, monothiophosphate (phosphorothioate), monodithiophosphate, phosphorothiolate, alpha-thiotriphosphate, beta-thiotriphosphate, or gamma-thiotriphosphate.

In some embodiments of any one of the aspects described herein, R^{25′} is a bond to an internucleotide linkage to a preceding nucleotide, hydroxyl, protected hydroxyl, optionally substituted C₂₋₃₀alkenyl, optionally substituted C₁₋₃₀ alkoxy or a vinylphosphonate (VP) group.

In some embodiments of any one of the aspects described herein, R^{25′} is a bond to an internucleotide linkage to a preceding nucleotide.

In some embodiments of any one of the aspects described herein, R^{25′} is a hydroxyl or protected hydroxyl.

In some embodiments of any one of the aspects described herein, R^{25′} is optionally substituted C₂₋₃₀alkenyl or optionally substituted C₁₋₃₀ alkoxy.

In some embodiments of any one of the aspects described herein, R^{25′} is a vinylphosphonate group.

In some embodiments of any one of the aspects described herein, the methylene connecting the R^{25′} to the rest of the nucleoside of Formula (II′) is absent and R^{25′} is connected directly to the rest of the nucleoside of Formula (II′).

In some embodiments of any one of the aspects described herein, R^{25′} is —CH(R⁵¹)—X⁵—R⁵², where X⁵ is absent, a bond or O; R⁵¹ is hydrogen, optionally substituted C₁₋₃₀alkyl, optionally substituted —C₂₋₃₀alkenyl, or optionally substituted —C₂₋₃₀alkynyl, and R⁵² is a bond to an internucleoside linkage to the preceding nucleotide.

In some embodiments of any one of the aspects described herein, X⁵ is O or a bond. For example, X⁵ is O. In some other embodiments of any one of the aspects described herein, X⁵ is absent, i.e., R^{25′} is —CH(R⁵¹)R.

In some embodiments of the any one of the aspects described herein, R^{25′} is —CH(R⁵¹)—R² or —C(R⁵¹)=CHR⁵², where R⁵¹ is hydrogen, optionally substituted C₁₋₃₀alkyl, optionally substituted —C₂₋₃₀alkenyl, or optionally substituted —C₂₋₃₀alkynyl, and R⁵² is a bond to an internucleoside linkage to the preceding nucleotide.

In some embodiments of the any one of the aspects described herein, R^{25′} is —CH(R⁵¹)—X⁵—R⁵². For example, R^{25′} is —CH(R⁵¹)—X⁵—R⁵² and where R⁵¹ is H or C₁-C₃₀alkyl optionally substituted with 1, 2, 3, 4 or 5 substituents independently selected from OH, CN, SC(O)Ph, oxo (═O), SH, SO₂NH₂, SO₂(C₁-C₄)alkyl, SO₂NH(C₁-C₄)alkyl, halogen, carbonyl, thiol, cyano, NH₂, NH(C₁-C₄)alkyl, N[(C₁-C₄)alkyl]₂, C(O)NH₂, COOH, COOMe, acetyl, (C₁-C₈)alkyl, O(C₁-C₈)alkyl (i.e., C₁-C₈alkoxy), O(C₁-C₈)haloalkyl, (C₂-C₈)alkenyl, (C₂-C₈)alkynyl, haloalkyl, thioalkyl, cyanomethylene, alkylaminyl, aryl, heteroaryl, substituted aryl, NH₂—C(O)-alkylene, NH(Me)-C(O)-alkylene, CH₂—C(O)-alkyl, C(O)-alkyl, alkylcarbonylaminyl, CH₂—[CH(OH)]ₘ—(CH₂)ₚ—OH, CH₂—[CH(OH)]ₘ—(CH₂)ₚ—NH₂ or CH₂-aryl-alkoxy, where “m” and “p” are independently 1, 2, 3, 4, 5 or 6. For example, R⁵¹ is H. In some other non-limiting examples, R⁵¹ is C₁-C₃₀alkyl optionally substituted with a NH₂, OH, C(O)NH₂, COOH, halo, SH, or C₁-C₆alkoxy.

In some embodiments of the any one of the aspects described herein, R^{25′} is —CH(R⁵¹)—O—R⁵², where R⁵¹ is H or C₁-C₃₀alkyl optionally substituted with 1, 2, 3, 4 or 5 substituents independently selected from 1, 2, 3, 4 or 5 substituents independently selected from OH, CN, SC(O)Ph, oxo (═O), SH, SO₂NH₂, SO₂(C₁-C₄)alkyl, SO₂NH(C₁-C₄)alkyl, halogen, carbonyl, thiol, cyano, NH₂, NH(C₁-C₄)alkyl, N[(C₁-C₄)alkyl]₂, C(O)NH₂, COOH, COOMe, acetyl, (C₁-C₈)alkyl, O(C₁-C₈)alkyl (i.e., C₁-C₈alkoxy), O(C₁-C₈)haloalkyl, (C₂-C₈)alkenyl, (C₂-C₈)alkynyl, haloalkyl, thioalkyl, cyanomethylene, alkylaminyl, aryl, heteroaryl, substituted aryl, NH₂—C(O)-alkylene, NH(Me)-C(O)-alkylene, CH₂—C(O)-alkyl, C(O)-alkyl, alkylcarbonylaminyl, CH₂—[CH(OH)]ₘ—(CH₂)ₚ—OH, CH₂—[CH(OH)]ₘ—(CH₂)ₚ—NH₂ or CH₂-aryl-alkoxy, where “m” and “p” are independently 1, 2, 3, 4, 5 or 6. For example, R⁵¹ is H. In some other non-limiting examples, R⁵¹ is C₁-C₃₀alkyl optionally substituted with a NH₂, OH, C(O)NH₂, COOH, halo, SH, or C₁-C₆alkoxy.

In some embodiments of any one of the aspects described herein, R^{25′} is —C(R⁵¹)=CHR⁵². It is noted that the double bond in —C(R⁵¹)=CHR⁵² can be in the cis or trans configuration. Accordingly, in some embodiments of any one of the aspects, R^{25′} is —C(R⁵¹)=CHR⁵² and wherein the double bond is in the cis configuration. In some other embodiments of any one of the aspects, R^{25′} is —C(R⁵¹)=CHR⁵² and wherein the double bond is in the trans configuration. In some embodiments of any one of the aspects described herein, R^{25′} is —CH═CHR⁵².

In some embodiments of any one of the aspects described herein, R⁵² is a bond to an internucleoside linkage to the preceding nucleotide.

In embodiments of the any one of the aspects described herein, R^{25′} is optionally substituted C₁₋₆alkyl-R⁵³, optionally substituted —C₂₋₆alkenyl-R⁵³, or optionally substituted —C₂₋₆alkynyl-R⁵³. In embodiments of the any one of the aspects described herein, R⁵³ can be —OR⁵⁴, —SR⁵⁵, —P(O)(OR⁵⁶)₂, —P(S)(OR⁵⁶)₂, —P(S)(SR⁵⁷)(OR⁵⁶), —P(S)(SR⁵⁷)₂, —OP(O)(OR⁵⁶)₂, —OP(S)(OR⁵⁶)₂, —OP(S)(SR⁵⁷)(OR⁵⁶), —OP(S)(SR⁵⁷)₂, —SP(O)(OR⁵⁶)₂, —SP(S)(OR⁵⁶)₂, —SP(S)(SR⁵⁷)(OR⁵⁶), or —SP(S)(SR⁵⁷)₂; where R⁵⁴ is hydrogen or oxygen protecting group; R⁵⁵ is hydrogen or sulfur protecting group; each R⁵⁶ is independently hydrogen, optionally substituted C₁₋₃₀alkyl, optionally substituted C₂₋₃₀alkenyl, or optionally substituted C₂₋₃₀alkynyl, or an oxygen-protecting group; and each R⁵⁷ is independently hydrogen, optionally substituted C₁₋₃₀alkyl, optionally substituted C₂₋₃₀alkenyl, or optionally substituted C₂₋₃₀alkynyl, or a sulfur-protecting group.

In some embodiments of any one of the aspects, at least one R⁵⁶ in —P(O)(OR⁵⁶)₂, —P(S)(OR⁵⁶)₂, —P(S)(SR⁵⁷)(OR⁵⁶), —OP(O)(OR⁵⁶)₂, —OP(S)(OR⁵⁶)₂, —OP(S)(SR⁵⁷)(OR⁵⁶), SP(O)(OR⁵⁶)₂, —SP(S)(OR⁵⁶)₂, and —SP(S)(SR⁵⁷)(OR⁵⁶) is hydrogen.

In some other embodiments of any one of the aspects, at least one R⁵⁶ in —P(O)(OR⁵⁶)₂, —P(S)(OR⁵⁶)₂, —P(S)(SR⁵⁷)(OR⁵⁶), —OP(O)(OR⁵⁶)₂, —OP(S)(OR⁵⁶)₂, —OP(S)(SR⁵⁷)(OR⁵⁶), SP(O)(OR⁵⁶)₂, —SP(S)(OR⁵⁶)₂, or —SP(S)(SR⁵⁷)(OR⁵⁶) is not hydrogen. For example, at least one at least one R⁵⁶ in P(O)(OR⁵⁶)₂, —P(S)(OR⁵⁶)₂, —P(S)(SR⁵⁷)(OR⁵⁶), —OP(O)(OR⁵⁶)₂, —OP(S)(OR⁵⁶)₂, —OP(S)(SR⁵⁷)(OR⁵⁶), SP(O)(OR⁵⁶)₂, —SP(S)(OR⁵⁶)₂, and —SP(S)(SR⁵⁷)(OR⁵⁶) is optionally substituted C₁₋₃₀alkyl, optionally substituted C₂₋₃₀alkenyl, or optionally substituted C₂₋₃₀alkynyl, or an oxygen-protecting group.

In some embodiments of any one of the aspects, at least one R⁵⁶ is H and at least one R⁵⁶ is other than H in —P(O)(OR⁵⁶)₂, —P(S)(OR⁵⁶)₂, —P(S)(SR⁵⁷)(OR⁵⁶), —OP(O)(OR⁵⁶)₂, —OP(S)(OR⁵⁶)₂, —OP(S)(SR⁵⁷)(OR⁵⁶), SP(O)(OR⁵⁶)₂, —SP(S)(OR⁵⁶)₂, and —SP(S)(SR⁵⁷)(OR⁵⁶).

In some embodiments of any one of the aspects, all R⁵⁶ are H in —P(O)(OR⁵⁶)₂, —P(S)(OR⁵⁶)₂, —P(S)(SR⁵⁷)(OR⁵⁶), —OP(O)(OR⁵⁶)₂, —OP(S)(OR⁵⁶)₂, —OP(S)(SR⁵⁷)(OR⁵⁶), —OP(S)(SR⁵⁷)₂, —SP(O)(OR⁵⁶)₂, —SP(S)(OR⁵⁶)₂, —SP(S)(SR⁵⁷)(OR⁵⁶), and —SP(S)(SR⁵⁷)₂.

In some embodiments of any one of the aspects, all R⁵⁶ are other than H in in —P(O)(OR⁵⁶)₂, —P(S)(OR⁵⁶)₂, —P(S)(SR⁵⁷)(OR⁵⁶), —OP(O)(OR⁵⁶)₂, —OP(S)(OR⁵⁶)₂, —OP(S)(SR⁵⁷)(OR⁵⁶), —OP(S)(SR⁵⁷)₂, —SP(O)(OR⁵⁶)₂, —SP(S)(OR⁵⁶)₂, —SP(S)(SR⁵⁷)(OR⁵⁶), and —SP(S)(SR⁵⁷)₂.

In some embodiments of any one of the aspects, at least one R⁵⁷ in —P(S)(SR⁵⁷)(OR⁵⁶)—P(S)(SR⁵⁷)₂, —OP(S)(OR⁵⁶)₂, —OP(S)(SR⁵⁷)(OR⁵⁶), —OP(S)(SR⁵⁷)₂, —SP(S)(SR⁵⁷)(OR⁵⁶), and —SP(S)(SR⁵⁷)₂ is H.

In some embodiments of any one of the aspects, at least one R⁵⁷ in —P(S)(SR⁵⁷)(OR⁵⁶)—P(S)(SR⁵⁷)₂, —OP(S)(OR⁵⁶)₂, —OP(S)(SR⁵⁷)(OR⁵⁶), —OP(S)(SR⁵⁷)₂, —SP(S)(SR⁵⁷)(OR⁵⁶), and —SP(S)(SR⁵⁷)₂ is other than H. For example, at least one R⁵⁷ in —P(S)(SR⁵⁷)(OR⁵⁶), —P(S)(SR⁵⁷)₂, —OP(S)(OR⁵⁶)₂, —OP(S)(SR⁵⁷)(OR⁵⁶), —OP(S)(SR⁵⁷)₂, —SP(S)(SR⁵⁷)(OR⁵⁶), and —SP(S)(SR⁵⁷)₂ is optionally substituted C₁₋₃₀alkyl, optionally substituted C₂₋₃₀alkenyl, or optionally substituted C₂₋₃₀alkynyl, or an sulfur-protecting group.

In some embodiments of any one of the aspects, at least one R⁵⁷ is H and at least one R⁵⁷ is other than H in —P(S)(SR⁵⁷)₂, —OP(S)(SR⁵⁷)₂ and —SP(S)(SR⁵⁷)₂.

In some embodiments, all R⁵⁷ are H in —P(S)(SR⁵⁷)(OR⁵⁶), —P(S)(SR⁵⁷)₂, —OP(S)(OR⁵⁶)₂, —OP(S)(SR⁵⁷)(OR⁵⁶), —OP(S)(SR⁵⁷)₂, —SP(S)(SR⁵⁷)(OR⁵⁶), and —SP(S)(SR⁵⁷)₂.

In some embodiments, all R⁵⁷ are other than H in —P(S)(SR⁵⁷)(OR⁵⁶), —P(S)(SR⁵⁷)₂, —OP(S)(OR⁵⁶)₂, —OP(S)(SR⁵⁷)(OR⁵⁶), —OP(S)(SR⁵⁷)₂, —SP(S)(SR⁵⁷)(OR⁵⁶), and —SP(S)(SR⁵⁷)₂.

In some embodiments of any one of the aspects described herein, R^{25′} is optionally substituted —C₂₋₆alkenyl-R⁵³. For example, R^{25′} is —C₂₋₆alkenyl-R⁵³, where C₂₋₆alkenyl is optionally substituted with 1, 2, 3, 4 or 5 substituents independently selected from OH, CN, SC(O)Ph, oxo (═O), SH, SO₂NH₂, SO₂(C₁-C₄)alkyl, SO₂NH(C₁-C₄)alkyl, halogen, carbonyl, thiol, cyano, NH₂, NH(C₁-C₄)alkyl, N[(C₁-C₄)alkyl]₂, C(O)NH₂, COOH, COOMe, acetyl, (C₁-C₈)alkyl, O(C₁-C₈)alkyl (i.e., C₁-C₈alkoxy), O(C₁-C₈)haloalkyl, (C₂-C₈)alkenyl, (C₂-C₈)alkynyl, haloalkyl, thioalkyl, cyanomethylene, alkylaminyl, aryl, heteroaryl, substituted aryl, NH₂—C(O)-alkylene, NH(Me)-C(O)-alkylene, CH₂—C(O)-alkyl, C(O)-alkyl, alkylcarbonylaminyl, CH₂—[CH(OH)]ₘ—(CH₂)ₚ—OH, CH₂—[CH(OH)]ₘ—(CH₂)ₚ—NH₂ or CH₂-aryl-alkoxy, where “m” and “p” are independently 1, 2, 3, 4, 5 or 6; and R⁵³ is —P(O)(OR⁵⁶)₂, —P(S)(OR⁵⁶)₂, —P(S)(SR⁵⁷)(OR⁵⁶), —P(S)(SR⁵⁷)₂, —OP(O)(OR⁵⁶)₂, —OP(S)(OR⁵⁶)₂, —OP(S)(SR⁵⁷)(OR⁵⁶), —OP(S)(SR⁵⁷)₂, —SP(O)(OR⁵⁶)₂, —SP(S)(OR⁵⁶)₂, —SP(S)(SR⁵⁷)(OR⁵⁶), or —SP(S)(SR⁵⁷)₂.

In some embodiments of any one of the aspects, R^{25′} is —CH═CHR⁵³. It is noted that a double bond in the optionally substituted —C₂₋₆alkenyl-R⁵³ can be in the cis or trans configuration. Accordingly, in some embodiments of any one of the aspects, R^{25′} is —CH═CHR⁵³ and wherein the double bond is in the cis configuration. In some other embodiments of any one of the aspects, R^{25′} is —CH═CHR⁵³ and wherein the double bond is in the trans configuration.

In some embodiments of any one of the aspects, R^{25′} is —CH═CH—P(O)(OR⁵⁶)₂, —CH═CH—P(S)(OR⁵⁶)₂, —CH═CH—P(S)(SR⁵⁷)(OR⁵⁶), —CH═CH—P(S)(SR⁵⁷)₂, —CH═CH—OP(O)(OR⁵⁶)₂, —CH═CH—OP(S)(OR⁵⁶)₂, —CH═CH—OP(S)(SR⁵⁷)(OR⁵⁶), —CH═CH—OP(S)(SR⁵⁷)₂, —CH═CH—SP(O)(OR⁵⁶)₂, —CH═CH—SP(S)(OR⁵⁶)₂, —CH═CH—SP(S)(SR⁵⁷)(OR⁵⁶), or —CH═CH—SP(S)(SR⁵⁷)₂. For example, R^{25′} is —CH═CH—P(O)(OR⁵⁶)₂.

In some embodiments, of any one of the aspects, R⁵⁴ is hydrogen or an oxygen protecting group. For example, R⁵⁴ is hydrogen or 4,4′-dimethoxytrityl (DMT). In some preferred embodiments, R⁵⁴ is H.

In some embodiments of any one of the aspects described herein, R^{25′} is optionally substituted —C₁₋₆alkenyl-R⁵³. For example, R^{25′} is —C₁₋₆alkenyl-R⁵³, where C₁₋₆alkenyl is optionally substituted with 1, 2, 3, 4 or 5 substituents independently selected from OH, CN, SC(O)Ph, oxo (═O), SH, SO₂NH₂, SO₂(C₁-C₄)alkyl, SO₂NH(C₁-C₄)alkyl, halogen, carbonyl, thiol, cyano, NH₂, NH(C₁-C₄)alkyl, N[(C₁-C₄)alkyl]₂, C(O)NH₂, COOH, COOMe, acetyl, (C₁-C₈)alkyl, O(C₁-C₈)alkyl (i.e., C₁-C₈alkoxy), O(C₁-C₈)haloalkyl, (C₂-C₈)alkenyl, (C₂-C₈)alkynyl, haloalkyl, thioalkyl, cyanomethylene, alkylaminyl, aryl, heteroaryl, substituted aryl, NH₂—C(O)-alkylene, NH(Me)-C(O)-alkylene, CH₂—C(O)-alkyl, C(O)-alkyl, alkylcarbonylaminyl, CH₂—[CH(OH)]ₘ—(CH₂)ₚ—OH, CH₂—[CH(OH)]ₘ—(CH₂)ₚ—NH₂ or CH₂-aryl-alkoxy, where “m” and “p” are independently 1, 2, 3, 4, 5 or 6; and R⁵³ is —OR⁵⁴, —SR⁵⁵, —P(O)(OR⁵⁶)₂, —P(S)(OR⁵⁶)₂, —P(S)(SR⁵⁷)(OR⁵⁶), —P(S)(SR⁵⁷)₂, —OP(O)(OR⁵⁶)₂, —OP(S)(OR⁵⁶)₂, —OP(S)(SR⁵⁷)(OR⁵⁶), —OP(S)(SR⁵⁷)₂, —SP(O)(OR⁵⁶)₂, —SP(S)(OR⁵⁶)₂, —SP(S)(SR⁵⁷)(OR⁵⁶), or —SP(S)(SR⁵⁷)₂.

In some embodiments of any one of the aspects described herein, R^{25′} can be —CH(R⁵⁸)—R⁵³, where R⁵³ is —OR⁵⁴, —SR⁵⁵, —P(O)(OR⁵⁶)₂, —P(S)(OR⁵⁶)₂, —P(S)(SR⁵⁷)(OR⁵⁶), —P(S)(SR⁵⁷)₂, —OP(O)(OR⁵⁶)₂, —OP(S)(OR⁵⁶)₂, —OP(S)(SR⁵⁷)(OR⁵⁶), —OP(S)(SR⁵⁷)₂, —SP(O)(OR⁵⁶)₂, —SP(S)(OR⁵⁶)₂, —SP(S)(SR⁵⁷)(OR⁵⁶), or —SP(S)(SR⁵⁷)₂; and R⁵⁸ is H, optionally substituted C₁₋₃₀alkyl, optionally substituted C₂₋₃₀alkenyl, or optionally substituted C₂₋₃₀alkynyl.

In some embodiments of any one of the aspects described herein, R⁵⁸ is H or C₁-C₃₀alkyl optionally substituted with 1, 2, 3, 4 or 5 substituents independently selected from OH, CN, SC(O)Ph, oxo (═O), SH, SO₂NH₂, SO₂(C₁-C₄)alkyl, SO₂NH(C₁-C₄)alkyl, halogen, carbonyl, thiol, cyano, NH₂, NH(C₁-C₄)alkyl, N[(C₁-C₄)alkyl]₂, C(O)NH₂, COOH, COOMe, acetyl, (C₁-C₈)alkyl, O(C₁-C₈)alkyl (i.e., C₁-C₈alkoxy), O(C₁-C₈)haloalkyl, (C₂-C₈)alkenyl, (C₂-C₈)alkynyl, haloalkyl, thioalkyl, cyanomethylene, alkylaminyl, aryl, heteroaryl, substituted aryl, NH₂—C(O)-alkylene, NH(Me)-C(O)-alkylene, CH₂—C(O)-alkyl, C(O)-alkyl, alkylcarbonylaminyl, CH₂—[CH(OH)]ₘ—(CH₂)ₚ—OH, CH₂—[CH(OH)]ₘ—(CH₂)ₚ—NH₂ or CH₂-aryl-alkoxy, where “m” and “p” are independently 1, 2, 3, 4, 5 or 6. In one non-limiting example, R⁵⁸ is H. In some other non-limiting examples, R⁵⁸ is C₁-C₃₀alkyl optionally substituted with a substituent selected from NH₂, OH, C(O)NH₂, COOH, halo, SH, and C₁-C₆alkoxy.

In some embodiments of any one of the aspects described herein, R^{25′} is —CH(R⁵⁸)—O—R⁵⁹, where R⁵⁹ is H, —P(O)(OR⁵⁶)₂, —P(S)(OR⁵⁶)₂, —P(S)(SR⁵⁷)(OR⁵⁶), —P(S)(SR⁵⁷)₂, —OP(O)(OR⁵⁶)₂. For example, R^{25′} is —CH(R⁵⁸)—O—R⁵⁹, where R⁵⁸ is H or optionally substituted C₁-C₃₀alkyl and R⁵⁹ is H or —P(O)(OR⁵⁶)₂.

In some embodiments of any one of the aspects described herein, R^{25′} is —CH(R⁵⁸)—S—R⁶⁰, where R⁶⁰ is H, —P(O)(OR⁵⁶)₂, —P(S)(OR⁵⁶)₂, —P(S)(SR⁵⁷)(OR⁵⁶), —P(S)(SR⁵⁷)₂, —OP(O)(OR⁵⁶)₂.

It is noted that in nucleosides of Formula (II′) no more than one of R^{22′} and R^{23′} is a bond to an internucleotide linkage to a subsequent nucleotide, and when both of R^{22′} and R^{23′} are not a bond to an internucleotide linkage, then R^{25′} is a bond to an internucleotide linkage to a preceding nucleotide.

In some embodiments, one of R^{22′} and R^{23′} is —O(CH₂)ₙ₁—C(O)OR^{LV}, and the other of R^{22′} and R^{23′} is hydrogen, hydroxyl, protected hydroxyl, a bond to an internucleotide linkage to a subsequent nucleotide, a 3′-oligonucleotide capping group, a solid support, a linker or a linker covalently bonded a solid support, and R^{25′} is hydroxyl, protected hydroxyl, a bond to an internucleotide linkage to a preceding nucleotide or a vinyl phosphate group.

For example, R^{22′} is —O(CH₂)ₙ₁—C(O)OR^{LV}, R^{23′} is hydrogen, hydroxyl, protected hydroxyl, a bond to an internucleotide linkage to a subsequent nucleotide, a 3′-oligonucleotide capping group, a solid support, a linker or a linker covalently bonded a solid support, and R^{25′} is hydroxyl, protected hydroxyl, a bond to an internucleotide linkage to a preceding nucleotide or a vinyl phosphate group. In another non-limiting example, R^{23′} is —O(CH₂)ₙ₁—C(O)OR^{LV}, R^{22′} is hydrogen, hydroxyl, protected hydroxyl, a bond to an internucleotide linkage to a subsequent nucleotide, a 3′-oligonucleotide capping group, a solid support, a linker or a linker covalently bonded a solid support, and R^{25′} is hydroxyl, protected hydroxyl, a bond to an internucleotide linkage to a preceding nucleotide or a vinyl phosphate group.

In some embodiments, R^{22′} is —O(CH₂)ₙ₁—C(O)OR^{LV}, R^{23′} is a bond to an internucleotide linkage to a subsequent nucleotide, a linker or a linker covalently bonded a solid support, and R^{25′} is hydroxyl, protected hydroxyl, a bond to an internucleotide linkage to a preceding nucleotide or a vinyl phosphate group.

In some embodiments, R^{23′} is —O(CH₂)ₙ₁—C(O)OR^{LV}, and R^{22′} is a bond to an internucleotide linkage to a subsequent nucleotide, a linker or a linker covalently bonded a solid support, and R^{25′} is hydroxyl, protected hydroxyl, a bond to an internucleotide linkage to a preceding nucleotide or a vinyl phosphate group.

### R⁴

Embodiments of the any one of the aspects described herein include R⁴. In any one of the aspects described herein, R⁴ can be R^{MA}, hydrogen, optionally substituted C₁₋₆alkyl, optionally substituted C₂₋₆alkenyl, optionally substituted C₂₋₆alkynyl, or optionally substituted C₁₋₆alkoxy. For example, R⁴ can be R^{MA}, hydrogen, optionally substituted C₁₋₆alkyl or optionally substituted C₁₋₆alkoxy.

In some embodiments of any one of the aspects described herein, R⁴ is H.

In some embodiments of any one of the aspects described herein, R^{$} is —O(CH₂)ₘ₁—X^{M′}—R^{M′}. For example, R⁴ is —OCH₂CH₂—X^{M′}—R^{M′}

### R^{L}

In embodiments of the any one of the aspects described herein, each R^{L} can be independently selected from the groups consisting of H, carbohydrates, lipids, vitamins, peptides, proteins, lipoproteins, peptidomimetics, polyamines, nucelsides and nucleotides, oligonucleotides, detectable labels, diagnostic agents (e.g., bitoin), fluorescent dyes, polyethylene glycols (PEGs), antibodies, antibody fragments (e.g., nanobodies).

In some embodiments of any one of the aspects described herein, R^{L} is a ligand.

It is noted that when more than one R^{L} are present, they can be same or different. Accordingly, in some embodiments of any one of the aspects described herein, all R^{L} are same. In some other embodiments of any one of the aspects described herein, R^{L} are different. Ligands

Embodiments of the any one of the aspects described herein include a ligand. Exemplary ligands include, but are not limited to, carbohydrates, lipids, vitamins, peptides, proteins, lipoproteins, peptidomimetics, polyamines, nucelsides and nucleotides, oligonucleotides, detectable labels, diagnostic agents (e.g., bitoin), fluorescent dyes, polyethylene glycols (PEGs), antibodies, antibody fragments (e.g., nanobodies).

Without wishing to be bound by a theory, ligands modify one or more properties of the attached molecule (e.g., the oligonucleotide described herein) including but not limited to pharmacodynamic, pharmacokinetic, binding, absorption, cellular distribution, cellular uptake, charge and clearance. Ligands are routinely used in the chemical arts and are linked directly or via an optional linking moiety or linking group to a parent compound. A preferred list of ligands includes without limitation, intercalators, reporter molecules, polyamines, polyamides, polyethylene glycols, thioethers, polyethers, cholesterols, thiocholesterols, cholic acid moieties, folate, lipids, phospholipids, biotin, phenazine, phenanthridine, anthraquinone, adamantane, acridine, fluoresceins, rhodamines, coumarins and dyes.

Preferred ligands amenable to the present invention include lipid moieties such as a cholesterol moiety (Letsinger et al., Proc. Natl. Acad. Sci. USA, 1989, 86, 6553); cholic acid (Manoharan et al., Bioorg. Med. Chem. Lett., 1994, 4, 1053); a thioether, e.g., hexyl-S-tritylthiol (Manoharan et al., Ann. N.Y. Acad. Sci., 1992, 660, 306; Manoharan et al., Bioorg. Med. Chem. Let., 1993, 3, 2765); a thiocholesterol (Oberhauser et al., Nucl. Acids Res., 1992, 20, 533); an aliphatic chain, e.g., dodecandiol or undecyl residues (Saison-Behmoaras et al., EMBO J., 1991, 10, 111; Kabanov et al., FEBS Lett., 1990, 259, 327; Svinarchuk et al., Biochimie, 1993, 75, 49); a phospholipid, e.g., di-hexadecyl-rac-glycerol or triethylammonium-1,2-di-O-hexadecyl-rac-glycero-3-H-phosphonate (Manoharan et al., Tetrahedron Lett., 1995, 36, 3651; Shea et al., Nucl. Acids Res., 1990, 18, 3777); a polyamine or a polyethylene glycol chain (Manoharan et al., Nucleosides & Nucleotides, 1995, 14, 969); adamantane acetic acid (Manoharan et al., Tetrahedron Lett., 1995, 36, 3651); a palmityl moiety (Mishra et al., Biochim. Biophys. Acta, 1995, 1264, 229); or an octadecylamine or hexylamino-carbonyl-oxycholesterol moiety (Crooke et al., J. Pharmacol. Exp. Ther., 1996, 277, 923).

Ligands can include naturally occurring molecules, or recombinant or synthetic molecules. Exemplary ligands include, but are not limited to, polylysine (PLL), poly L-aspartic acid, poly L-glutamic acid, styrene-maleic acid anhydride copolymer, poly(L-lactide-co-glycolied) copolymer, divinyl ether-maleic anhydride copolymer, N-(2-hydroxypropyl)methacrylamide copolymer (HMPA), polyethylene glycol (PEG, e.g., PEG-2K, PEG-5K, PEG-10K, PEG-12K, PEG-15K, PEG-20K, PEG-40K), MPEG, [MPEG]₂, polyvinyl alcohol (PVA), polyurethane, poly(2-ethylacryllic acid), N-isopropylacrylamide polymers, polyphosphazine, polyethylenimine, cationic groups, spermine, spermidine, polyamine, pseudopeptide-polyamine, peptidomimetic polyamine, dendrimer polyamine, arginine, amidine, protamine, cationic lipid, cationic porphyrin, quaternary salt of a polyamine, thyrotropin, melanotropin, lectin, glycoprotein, surfactant protein A, mucin, glycosylated polyaminoacids, transferrin, bisphosphonate, polyglutamate, polyaspartate, aptamer, asialofetuin, hyaluronan, procollagen, immunoglobulins (e.g., antibodies), insulin, transferrin, albumin, sugar-albumin conjugates, intercalating agents (e.g., acridines), cross-linkers (e.g. psoralen, mitomycin C), porphyrins (e.g., TPPC4, texaphyrin, Sapphyrin), polycyclic aromatic hydrocarbons (e.g., phenazine, dihydrophenazine), artificial endonucleases (e.g., EDTA), lipophilic molecules (e.g, steroids, bile acids, cholesterol, cholic acid, adamantane acetic acid, 1-pyrene butyric acid, dihydrotestosterone, 1,3-Bis-O(hexadecyl)glycerol, geranyloxyhexyl group, hexadecylglycerol, borneol, menthol, 1,3-propanediol, heptadecyl group, palmitic acid, myristic acid, O3-(oleoyl)lithocholic acid, O3-(oleoyl)cholenic acid, dimethoxytrityl, or phenoxazine), peptides (e.g., an alpha helical peptide, amphipathic peptide, RGD peptide, cell permeation peptide, endosomolytic/fusogenic peptide), alkylating agents, phosphate, amino, mercapto, polyamino, alkyl, substituted alkyl, radiolabeled markers, enzymes, haptens (e.g. biotin), transport/absorption facilitators (e.g., naproxen, aspirin, vitamin E, folic acid), synthetic ribonucleases (e.g., imidazole, bisimidazole, histamine, imidazole clusters, acridine-imidazole conjugates, Eu3+ complexes of tetraazamacrocycles), dinitrophenyl, HRP, AP, antibodies, hormones and hormone receptors, lectins, carbohydrates, multivalent carbohydrates, vitamins (e.g., vitamin A, vitamin E, vitamin K, vitamin B, e.g., folic acid, B12, riboflavin, biotin and pyridoxal), vitamin cofactors, lipopolysaccharide, an activator of p38 MAP kinase, an activator of NF-κB, taxon, vincristine, vinblastine, cytochalasin, nocodazole, japlakinolide, latrunculin A, phalloidin, swinholide A, indanocine, myoservin, tumor necrosis factor alpha (TNFalpha), interleukin-1 beta, gamma interferon, natural or recombinant low density lipoprotein (LDL), natural or recombinant high-density lipoprotein (HDL), and a cell-permeation agent (e.g., a.helical cell-permeation agent).

Peptide and peptidomimetic ligands include those having naturally occurring or modified peptides, e.g., D or L peptides; α, β, or γ peptides; N-methyl peptides; azapeptides; peptides having one or more amide, i.e., peptide, linkages replaced with one or more urea, thiourea, carbamate, or sulfonyl urea linkages; or cyclic peptides. A peptidomimetic (also referred to herein as an oligopeptidomimetic) is a molecule capable of folding into a defined three-dimensional structure similar to a natural peptide. The peptide or peptidomimetic ligand can be about 5-50 amino acids long, e.g., about 5, 10, 15, 20, 25, 30, 35, 40, 45, or 50 amino acids long.

Exemplary amphipathic peptides include, but are not limited to, cecropins, lycotoxins, paradaxins, buforin, CPF, bombinin-like peptide (BLP), cathelicidins, ceratotoxins, S. clava peptides, hagfish intestinal antimicrobial peptides (HFIAPs), magainines, brevinins-2, dermaseptins, melittins, pleurocidin, H₂A peptides, Xenopus peptides, esculentinis-1, and caerins.

As used herein, the term “endosomolytic ligand” refers to molecules having endosomolytic properties. Endosomolytic ligands promote the lysis of and/or transport of the composition of the invention, or its components, from the cellular compartments such as the endosome, lysosome, endoplasmic reticulum (ER), Golgi apparatus, microtubule, peroxisome, or other vesicular bodies within the cell, to the cytoplasm of the cell. Some exemplary endosomolytic ligands include, but are not limited to, imidazoles, poly or oligoimidazoles, linear or branched polyethyleneimines (PEIs), linear and brached polyamines, e.g. spermine, cationic linear and branched polyamines, polycarboxylates, polycations, masked oligo or poly cations or anions, acetals, polyacetals, ketals/polyketals, orthoesters, linear or branched polymers with masked or unmasked cationic or anionic charges, dendrimers with masked or unmasked cationic or anionic charges, polyanionic peptides, polyanionic peptidomimetics, pH-sensitive peptides, natural and synthetic fusogenic lipids, natural and synthetic cationic lipids.

Exemplary endosomolytic/fusogenic peptides include, but are not limited to, AALEALAEALEALAEALEALAEAAAAGGC (GALA, SEQ ID NO: 42); AALAEALAEALAEALAEALAEALAAAAGGC (EALA, SEQ ID NO: 43); ALEALAEALEALAEA (SEQ ID NO: 44); GLFEAIEGFIENGWEGMIWDYG (INF-7, SEQ ID NO: 45); GLFGAIAGFIENGWEGMIDGWYG (Inf HA-2, SEQ ID NO: 46); GLFEAIEGFIENGWEGMIDGWYGCGLFEAIEGFIENGWEGMID GWYGC (diINF-7, SEQ ID NO: 47); GLFEAIEGFIENGWEGMIDGGCGLFEAIEGFIENGWEGMIDGGC (diINF-3, SEQ ID NO: 48); GLFGALAEALAEALAEHLAEALAEALEALAAGGSC (GLF, SEQ ID NO: 49); GLFEAIEGFIENGWEGLAEALAEALEALAAGGSC (GALA-INF3, SEQ ID NO: 50); GLF EAI EGFI ENGW EGnI DG K GLF EAI EGFI ENGW EGnI DG (INF-5, n is norleucine, SEQ ID NO: 51); LFEALLELLESLWELLLEA (JTS-1, SEQ ID NO: 52); GLFKALLKLLKSLWKLLLKA (ppTG1, SEQ ID NO: 53); GLFRALLRLLRSLWRLLLRA (ppTG20, SEQ ID NO: 54); WEAKLAKALAKALAKHLAKALAKALKACEA (KALA, SEQ ID NO: 55); GLFFEAIAEFIEGGWEGLIEGC (HA, SEQ ID NO: 56); GIGAVLKVLTTGLPALISWIKRKRQQ (Melittin, SEQ ID NO: 57); HsWYG (SEQ ID NO: 58); and CHK6HC (SEQ ID NO: 59).

Without wishing to be bound by theory, fusogenic lipids fuse with and consequently destabilize a membrane. Fusogenic lipids usually have small head groups and unsaturated acyl chains. Exemplary fusogenic lipids include, but are not limited to, 1,2-dileoyl-sn-3-phosphoethanolamine (DOPE), phosphatidylethanolamine (POPE), palmitoyloleoylphosphatidylcholine (POPC), (6Z,9Z,28Z,31Z)-heptatriaconta-6,9,28,31-tetraen-19-ol (Di-Lin), N-methyl(2,2-di((9Z,12Z)-octadeca-9,12-dienyl)-1,3-dioxolan-4-yl)methanamine (DLin-k-DMA) and N-methyl-2-(2,2-di((9Z,12Z)-octadeca-9,12-dienyl)-1,3-dioxolan-4-yl)ethanamine (also referred to as XTC herein).

Synthetic polymers with endosomolytic activity amenable to the present invention are described in U.S. Pat. App. Pub. Nos. 2009/0048410; 2009/0023890; 2008/0287630; 2008/0287628; 2008/0281044; 2008/0281041; 2008/0269450; 2007/0105804; 20070036865; and 2004/0198687, contents of which are hereby incorporated by reference in their entirety.

Exemplary cell permeation peptides include, but are not limited to, RQIKIWFQNRRMKWKK (penetratin); GRKKRRQRRRPPQC (Tat fragment 48-60); GALFLGWLGAAGSTMGAWSQPKKKRKV (signal sequence based peptide); LLIILRRRIRKQAHAHSK (PVEC); GWTLNSAGYLLKINLKALAALAKKIL (transportan); KLALKLALKALKAALKLA (amphiphilic model peptide); RRRRRRRRR (Arg9); KFFFKFFKFFK (Bacterial cell wall permeating peptide); LLGDFFRKSKEKIGKEFKRIVQRIKDFLRNLVPRTES (LL-37); SWLSKTAKKLENSAKKRISEGIAIAIQGGPR (cecropin P1); ACYCRIPACIAGERRYGTCIYQGRLWAFCC (α-defensin); DHYNCVSSGGQCLYSACPIFTKIQGTCYRGKAKCCK (β-defensin); RRRPRPPYLPRPRPPPFFPPRLPPRIPPGFPPRFPPRFPGKR-NH2 (PR-39); ILPWKWPWWPWRR-NH2 (indolicidin); AAVALLPAVLLALLAP (RFGF); AALLPVLLAAP (RFGF analogue); and RKCRIVVIRVCR (bactenecin).

Exemplary cationic groups include, but are not limited to, protonated amino groups, derived from e.g., O-AMINE (AMINE=NH₂; alkylamino, dialkylamino, heterocyclyl, arylamino, diaryl amino, heteroaryl amino, or diheteroaryl amino, ethylene diamine, polyamino); aminoalkoxy, e.g., O(CH₂)ₙAMINE, (e.g., AMINE=NH₂; alkylamino, dialkylamino, heterocyclyl, arylamino, diaryl amino, heteroaryl amino, or diheteroaryl amino, ethylene diamine, polyamino); amino (e.g. NH₂; alkylamino, dialkylamino, heterocyclyl, arylamino, diaryl amino, heteroaryl amino, diheteroaryl amino, or amino acid); and NH(CH₂CH₂NH)ₙCH₂CH₂-AMINE (AMINE=NH₂; alkylamino, dialkylamino, heterocyclyl, arylamino, diaryl amino, heteroaryl amino, or diheteroaryl amino).

As used herein the term “targeting ligand” refers to any molecule that provides an enhanced affinity for a selected target, e.g., a cell, cell type, tissue, organ, region of the body, or a compartment, e.g., a cellular, tissue or organ compartment. Some exemplary targeting ligands include, but are not limited to, antibodies, antigens, folates, receptor ligands, carbohydrates, aptamers, integrin receptor ligands, chemokine receptor ligands, transferrin, biotin, serotonin receptor ligands, PSMA, endothelin, GCPII, somatostatin, LDL and HDL ligands.

Carbohydrate based targeting ligands include, but are not limited to, D-galactose, multivalent galactose, N-acetyl-D-galactosamine (GalNAc), multivalent GalNAc, e.g. GalNAc2 and GalNAc3; D-mannose, multivalent mannose, multivalent lactose, N-acetyl-gulucosamine, multivalent fucose, glycosylated polyaminoacids and lectins. The term multivalent indicates that more than one monosaccharide unit is present. Such monosaccharide subunits can be linked to each other through glycosidic linkages or linked to a scaffold molecule.

A number of folate and folate analogs amenable to the present invention as ligands are described in U.S. Pat. Nos. 2,816,110; 5,552,545; 6,335,434 and 7,128,893, contents of which are herein incorporated in their entireties by reference.

As used herein, the terms “PK modulating ligand” and “PK modulator” refers to molecules which can modulate the pharmacokinetics of oligonucleotides described herein. Some exemplary PK modulator include, but are not limited to, lipophilic molecules, bile acids, sterols, phospholipid analogues, peptides, protein binding agents, vitamins, fatty acids, phenoxazine, aspirin, naproxen, ibuprofen, suprofen, ketoprofen, (S)-(+)-pranoprofen, carprofen, PEGs, biotin, and transthyretia-binding ligands (e.g., tetraiidothyroacetic acid, 2, 4, 6-triiodophenol and flufenamic acid). Oligomeric compounds that comprise a number of phosphorothioate intersugar linkages are also known to bind to serum protein, thus short oligomeric compounds, e.g. oligonucleotides of comprising from about 5 to 30 nucleotides (e.g., 5 to 25 nucleotides, preferably 5 to 20 nucleotides, e.g., 5, 6, 7, 8, 9, 10, 11, 12, 13, 14, 15, 16, 17, 18, 19, or 20 nucleotides), and that comprise a plurality of phosphorothioate linkages in the backbone are also amenable to the present invention as ligands (e.g. as PK modulating ligands). The PK modulating oligonucleotide can comprise at least 3, 4, 5, 6, 7, 8, 9, 10, 11, 12, 13, 14, 15 or more phosphorothioate and/or phosphorodithioate linkages. In some embodiments, all internucleoside linkages in PK modulating oligonucleotide are phosphorothioate and/or phosphorodithioates linkages. In addition, aptamers that bind serum components (e.g. serum proteins) are also amenable to the present invention as PK modulating ligands. Binding to serum components (e.g. serum proteins) can be predicted from albumin binding assays, such as those described in Oravcova, et al., Journal of Chromatography B (1996), 677: 1-27.

When two or more ligands are present, the ligands can all have same properties, all have different properties or some ligands have the same properties while others have different properties. For example, a ligand can have targeting properties, have endosomolytic activity or have PK modulating properties. In a preferred embodiment, all the ligands have different properties.

In some embodiments of any one of the aspects, the ligand has a structure shown in any of Formula (IV)-(VII):

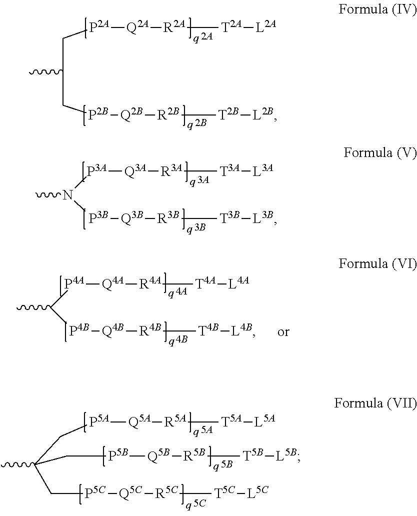

wherein:
q^{2A}, q^{2B}, q^{3A}, q^{3B}, q^{4A}, q^{4B}, q^{5A}, q^{5B} and q^{5C} represent independently for each occurrence 0-20 and wherein the repeating unit can be the same or different;
P^{2A}, P^{2B}, P^{3A}, P^{3B}, P^{4A}, P^{4B}, P^{5A}, P^{5B}, P^{5C}, T^{2A}, T^{2B}, T^{3A}, T^{3B}, T^{4A}, T^{4B}, T^{5A}, T^{5B}, T^{5C} are each independently for each occurrence absent, CO, NH, O, S, OC(O), NHC(O), CH₂, CH₂NH or CH₂O;
Q^{2A}, Q^{2B}, Q^{3A}, Q^{3B}, Q^{4A}, Q^{4B}, Q^{5A}, Q^{5B}, Q^{5C} are independently for each occurrence absent, alkylene, substituted alkylene wherein one or more methylenes can be interrupted or terminated by one or more of O, S, S(O), SO₂, N(R^{N}), C(R′)═C(R″), C≡C or C(O);
R^{2A}, R^{2B}, R^{3A}, R^{3B}, R^{4A}, R^{4B}, R^{5A}, R^{5B}, R^{5C} are each independently for each occurrence absent, NH, O, S, CH₂, C(O)O, C(O)NH, NHCH(Ra)C(O), —C(O)—CH(Ra)—NH—, CO, CH═N—O,

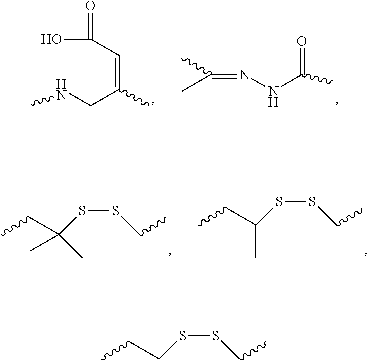

heterocyclyl;
L^{2A}, L^{2B}, L^{3A}, L^{3B}, L^{4A}, L^{4B}, L^{5A}, L^{5B} and L^{5C} represent the ligand; i.e. each independently for each occurrence a monosaccharide (such as GalNAc), disaccharide, trisaccharide, tetrasaccharide, oligosaccharide, or polysaccharide; and
R^{a} is H or amino acid side chain.

In some embodiments of any one of the aspects, the ligand is of Formula (VII):

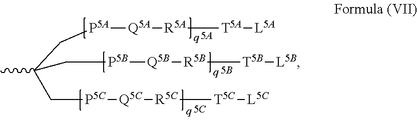

wherein L^{5A}, L^{5B} and L^{5C} represent a monosaccharide, such as GalNAc derivative.

Exemplary ligands include, but are not limited to, the following:

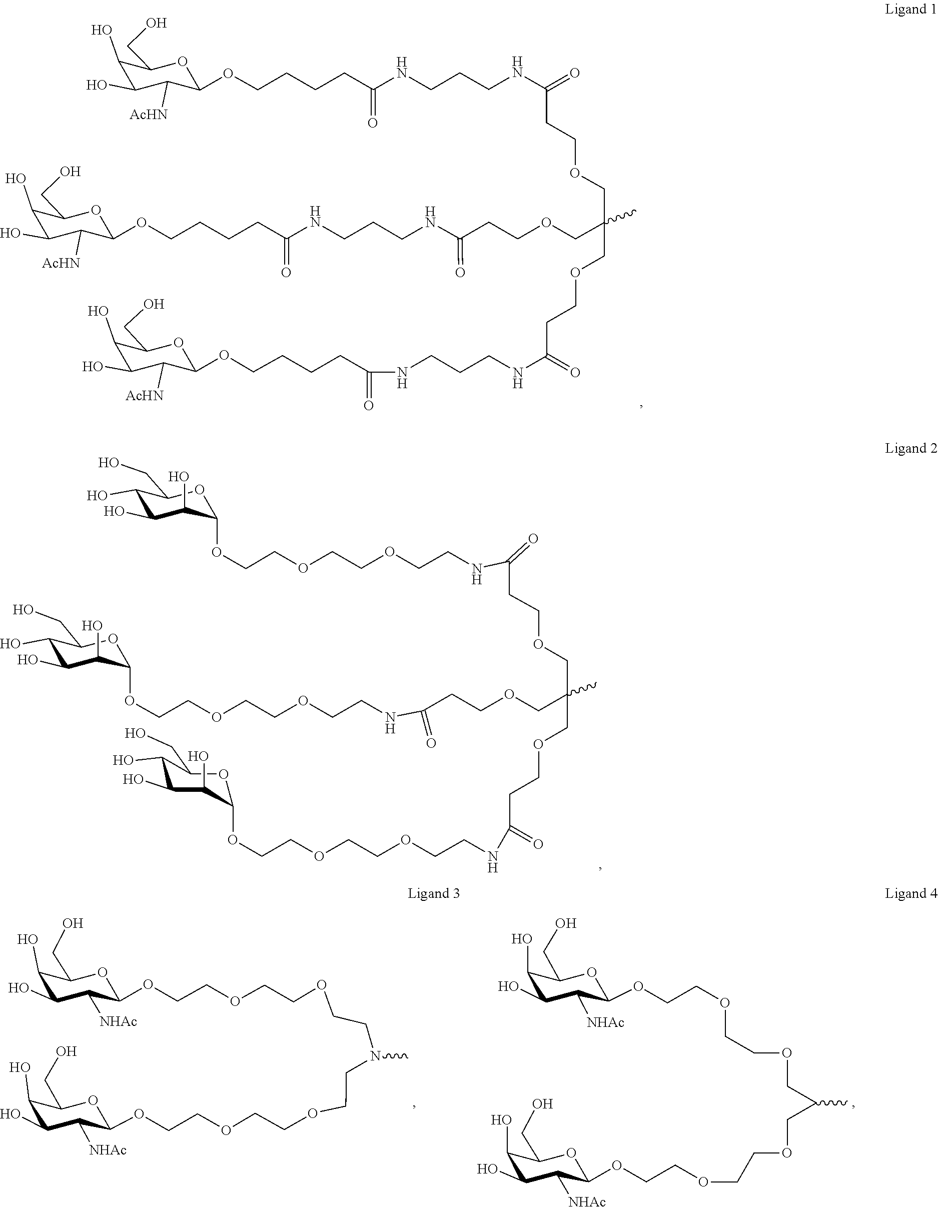

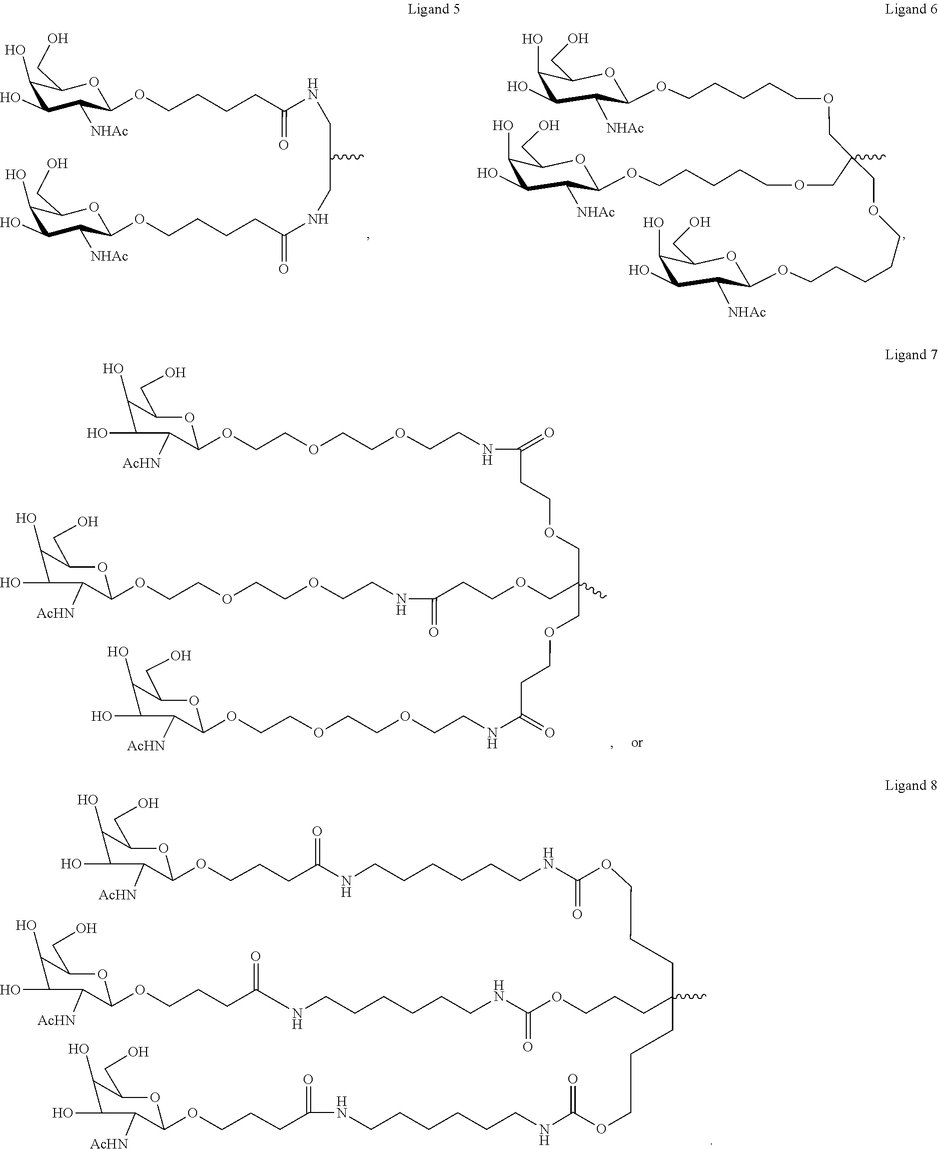

In some embodiments of any one of the aspects described herein, the ligand is a ligand described in U.S. Pat. No. 5,994,517 or U.S. Pat. No. 6,906,182, content of each of which is incorporated herein by reference in its entirety.

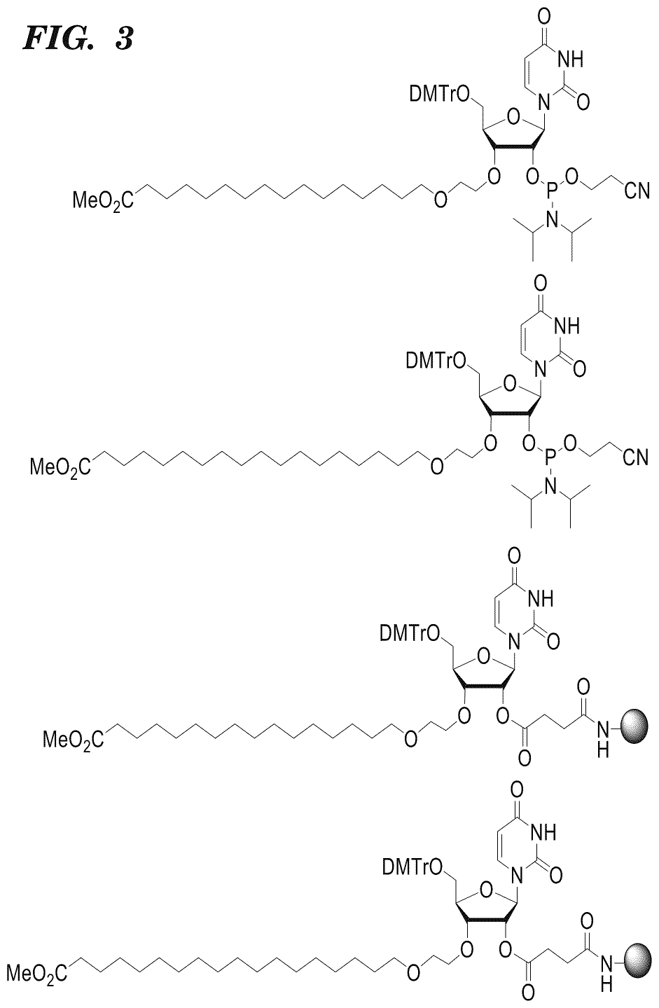

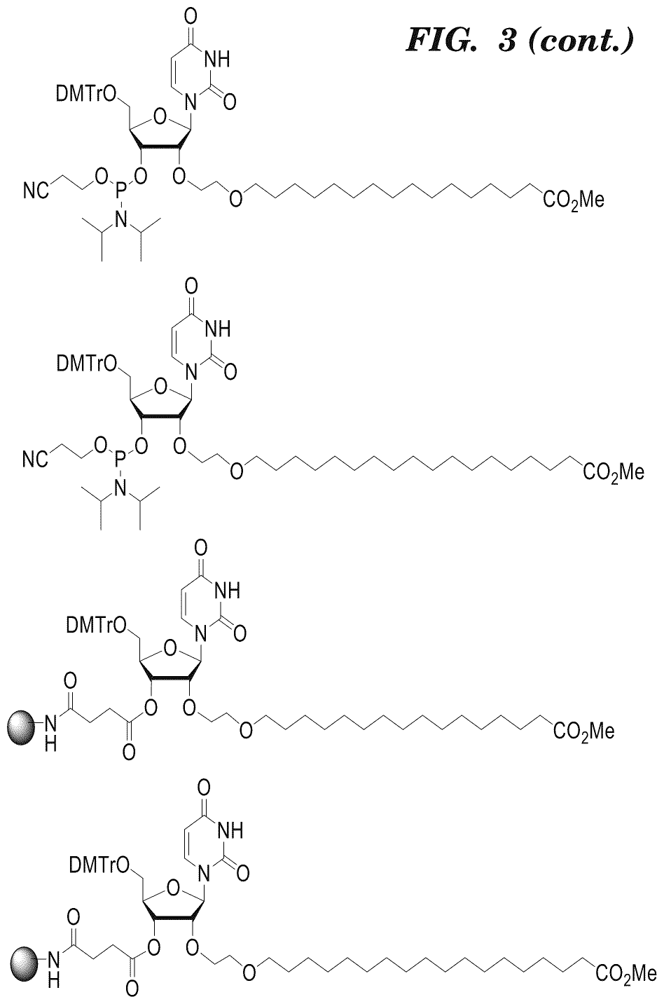

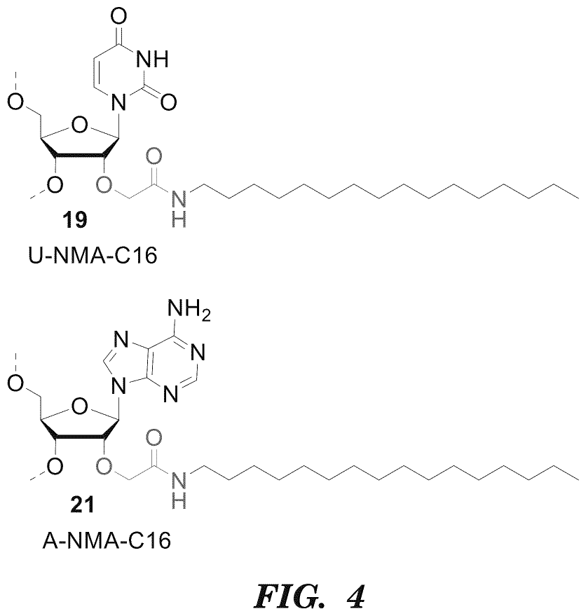

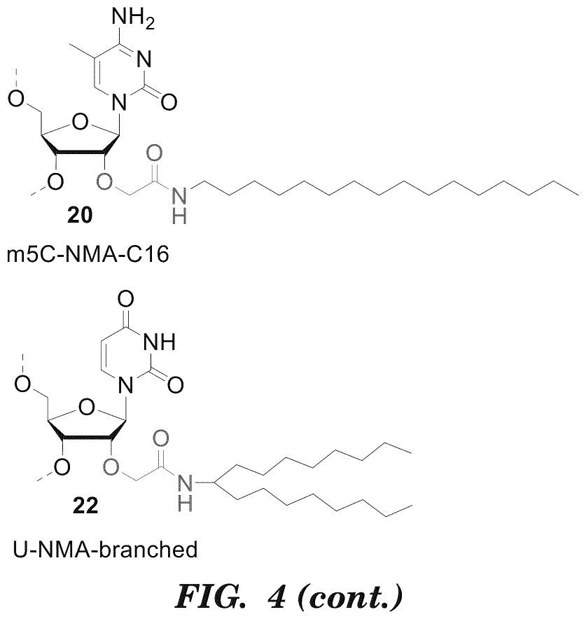

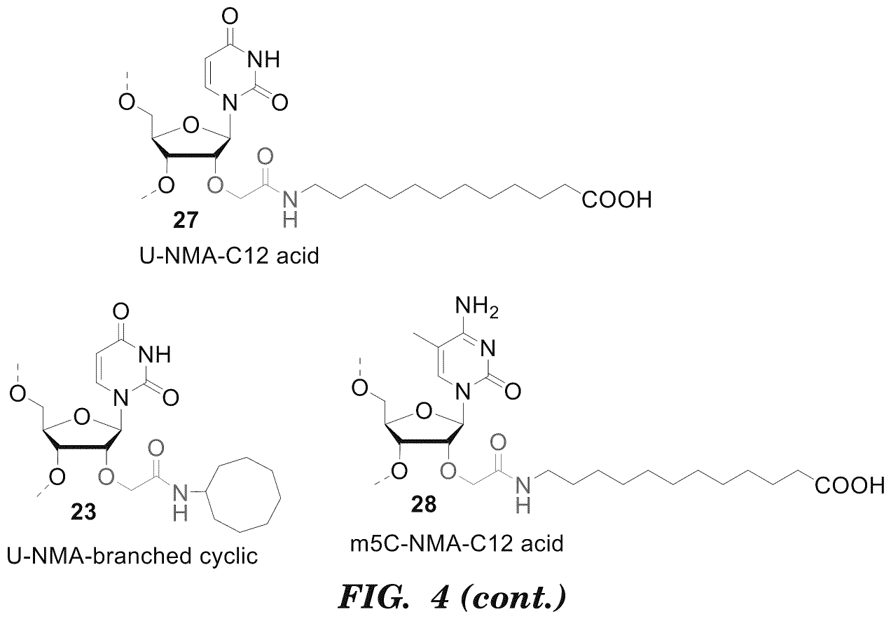

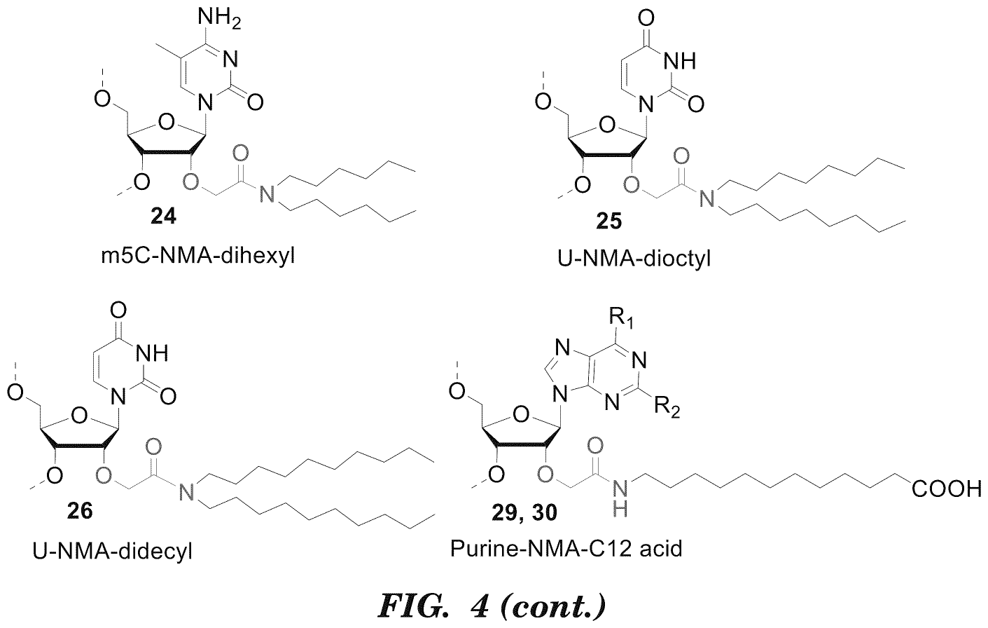

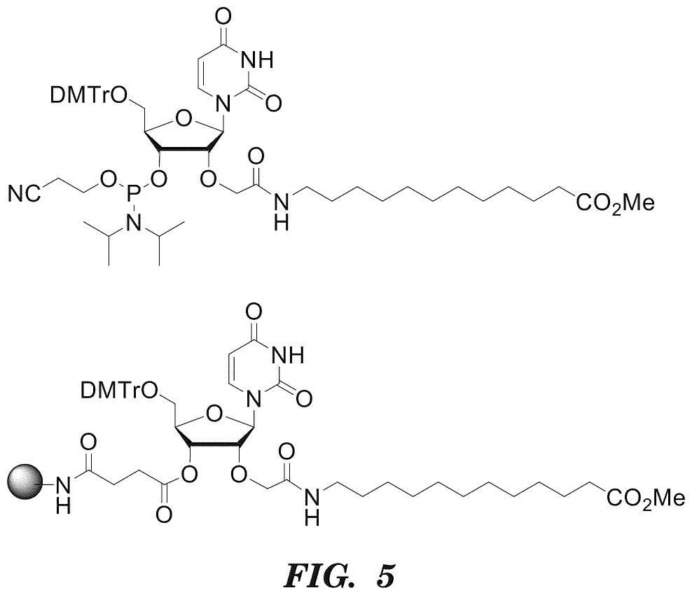

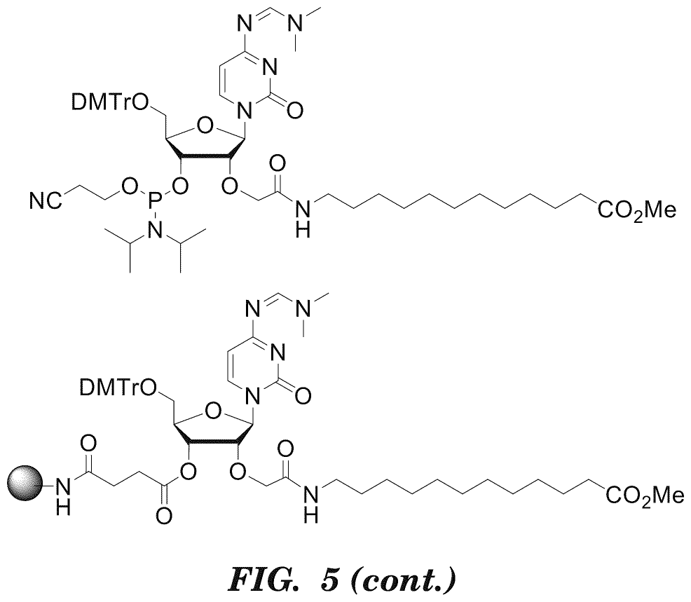

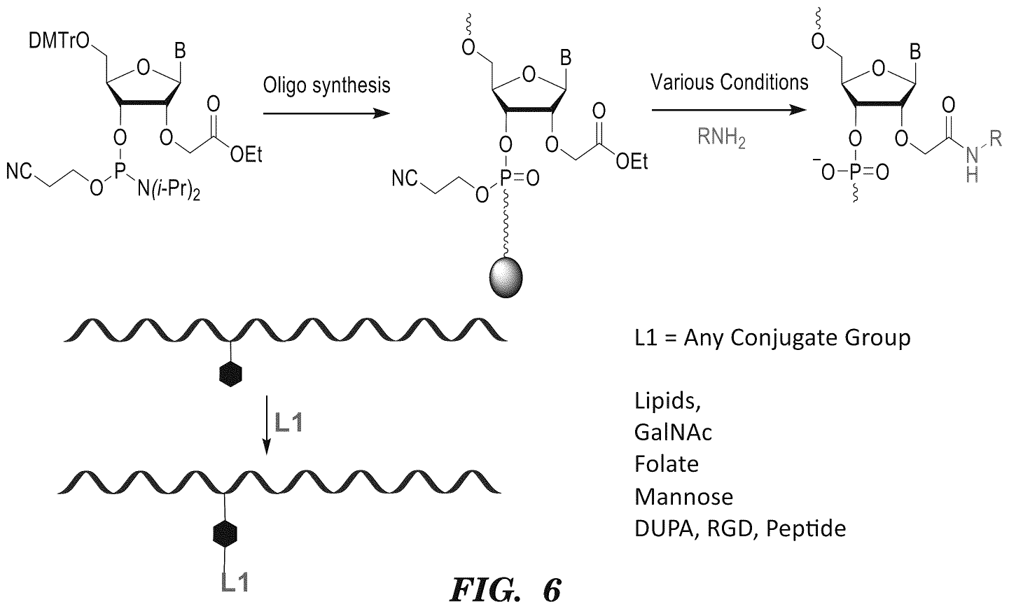

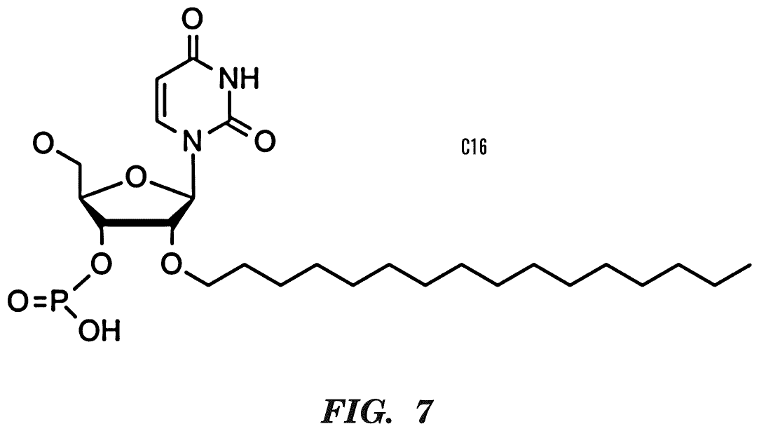

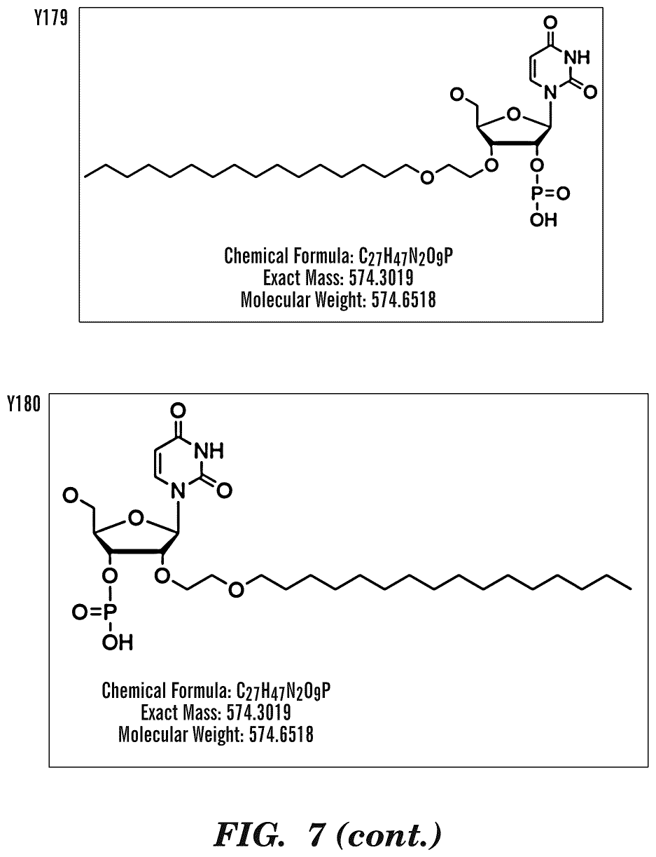

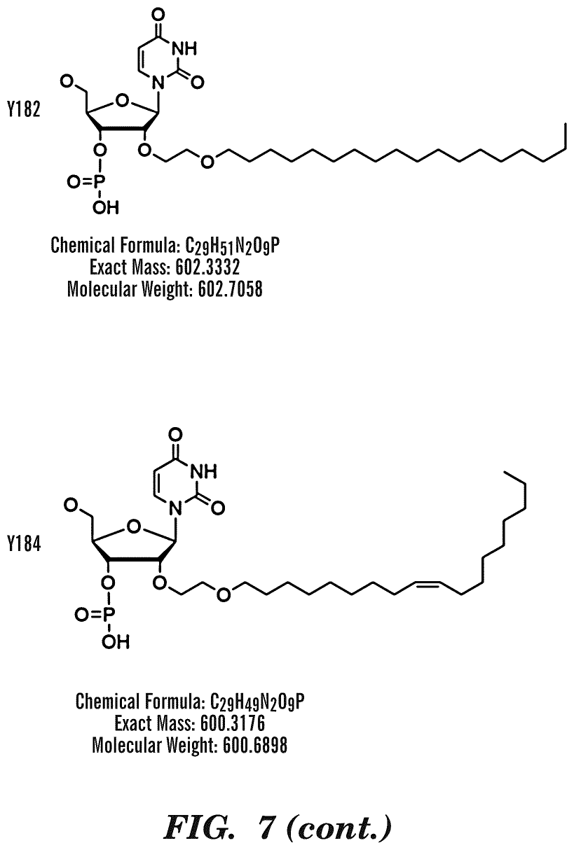

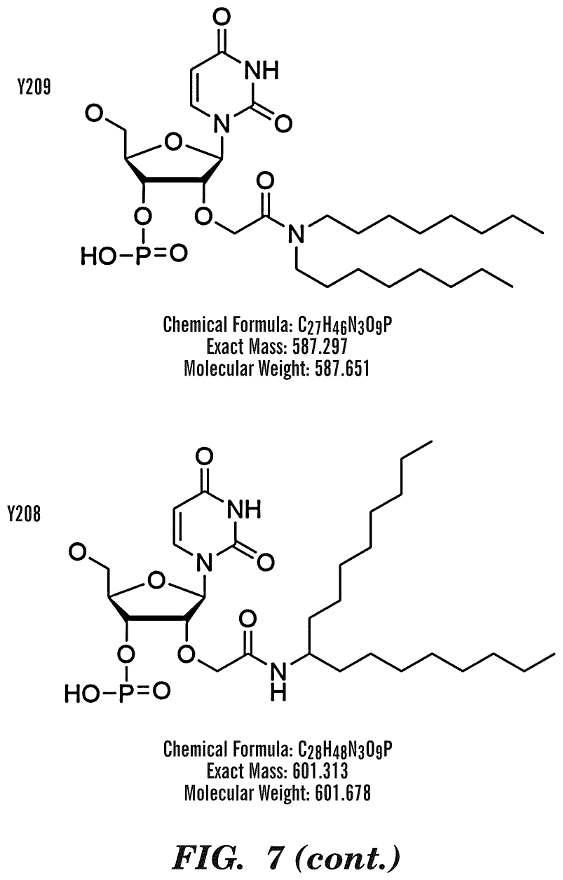

In some embodiments, the ligand can be a tri-antennary ligand described in FIG. 3 of U.S. Pat. No. 6,906,182. For example, the ligand is selected from the following tri-antennary ligands.

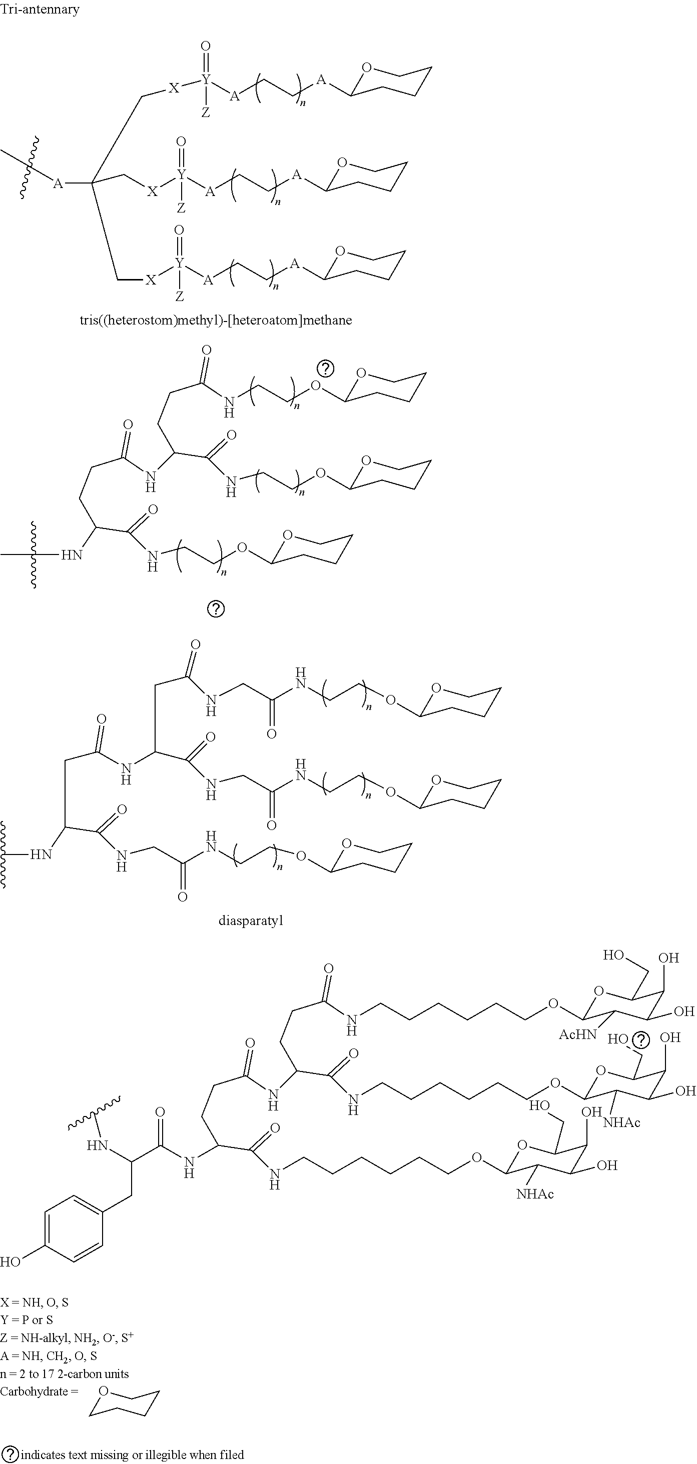

It is noted that when more than one ligands are present, they can be same or different. Accordingly, in some embodiments of any one of the aspects described herein, all ligands are same. In some other embodiments of any one of the aspects described herein, ligands are different.

### L (Linker)

Embodiments of the any one of the aspects described herein include L, a linker. As used herein, the term “linker” means an organic moiety that connects two parts of a compound. Linkers typically comprise a direct bond or an atom such as oxygen or sulfur, a unit such as NR¹, C(O), C(O)O, C(O)NR¹, SO, SO₂, SO₂NH or a chain of atoms, such as substituted or unsubstituted alkyl, substituted or unsubstituted alkenyl, substituted or unsubstituted alkynyl, arylalkyl, arylalkenyl, arylalkynyl, heteroarylalkyl, heteroarylalkenyl, heteroarylalkynyl, heterocyclylalkyl, heterocyclylalkenyl, heterocyclylalkynyl, aryl, heteroaryl, heterocyclyl, cycloalkyl, cycloalkenyl, alkylarylalkyl, alkylarylalkenyl, alkylarylalkynyl, alkenylarylalkyl, alkenylarylalkenyl, alkenylarylalkynyl, alkynylarylalkyl, alkynylarylalkenyl, alkynylarylalkynyl, alkylheteroarylalkyl, alkylheteroarylalkenyl, alkylheteroarylalkynyl, alkenylheteroarylalkyl, alkenylheteroarylalkenyl, alkenylheteroarylalkynyl, alkynylheteroarylalkyl, alkynylheteroarylalkenyl, alkynylheteroarylalkynyl, alkylheterocyclylalkyl, alkylheterocyclylalkenyl, alkylhererocyclylalkynyl, alkenylheterocyclylalkyl, alkenylheterocyclylalkenyl, alkenylheterocyclylalkynyl, alkynylheterocyclylalkyl, alkynylheterocyclylalkenyl, alkynylheterocyclylalkynyl, alkylaryl, alkenylaryl, alkynylaryl, alkylheteroaryl, alkenylheteroaryl, alkynylhereroaryl, where one or more methylenes can be interrupted or terminated by O, S, S(O), SO₂, N(R¹)₂, C(O), cleavable linking group, substituted or unsubstituted aryl, substituted or unsubstituted heteroaryl, substituted or unsubstituted heterocyclic; where R¹ is hydrogen, acyl, aliphatic or substituted aliphatic.

In some embodiments, the linker is a cleavable linker. Cleavable linkers are those that rely on processes inside a target cell to liberate the two parts the linker is holding together, as reduction in the cytoplasm, exposure to acidic conditions in a lysosome or endosome, or cleavage by specific enzymes (e.g. proteases) within the cell. As such, cleavable linkers allow the two parts to be released in their original form after internalization and processing inside a target cell. Cleavable linkers include, but are not limited to, those whose bonds can be cleaved by enzymes (e.g., peptide linkers); reducing conditions (e.g., disulfide linkers); or acidic conditions (e.g., hydrazones and carbonates).

Generally, the cleavable linker comprises at least one cleavable linking group. A cleavable linking group is one which is sufficiently stable outside the cell, but which upon entry into a target cell is cleaved to release the two parts the linker is holding together. In a preferred embodiment, the cleavable linking group is cleaved at least 10 times or more, preferably at least 100 times faster in the target cell or under a first reference condition (which can, e.g., be selected to mimic or represent intracellular conditions) than in the blood or serum of a subject, or under a second reference condition (which can, e.g., be selected to mimic or represent conditions found in the blood or serum).

Cleavable linking groups are susceptible to cleavage agents, e.g., pH, redox potential or the presence of degradative molecules. Generally, cleavage agents are more prevalent or found at higher levels or activities inside cells than in serum or blood. Examples of such degradative agents include: redox agents which are selected for particular substrates or which have no substrate specificity, including, e.g., oxidative or reductive enzymes or reductive agents such as mercaptans, present in cells, that can degrade a redox cleavable linking group by reduction; esterases; endosomes or agents that can create an acidic environment, e.g., those that result in a pH of five or lower; enzymes that can hydrolyze or degrade an acid cleavable linking group by acting as a general acid, peptidases (which can be substrate specific), and phosphatases.

A cleavable linkage group, such as a disulfide bond can be susceptible to pH. The pH of human serum is 7.4, while the average intracellular pH is slightly lower, ranging from about 7.1-7.3. Endosomes have a more acidic pH, in the range of 5.5-6.0, and lysosomes have an even more acidic pH at around 5.0. Some linkers will have a cleavable linking group that is cleaved at a preferred pH, thereby releasing the cationic lipid from the ligand inside the cell, or into the desired compartment of the cell.

A linker can include a cleavable linking group that is cleavable by a particular enzyme. The type of cleavable linking group incorporated into a linker can depend on the cell to be targeted. For example, liver targeting ligands can be linked to the cationic lipids through a linker that includes an ester group. Liver cells are rich in esterases, and therefore the linker will be cleaved more efficiently in liver cells than in cell types that are not esterase-rich. Other cell-types rich in esterases include cells of the lung, renal cortex, and testis. Linkers that contain peptide bonds can be used when targeting cell types rich in peptidases, such as liver cells and synoviocytes.

In general, the suitability of a candidate cleavable linking group can be evaluated by testing the ability of a degradative agent (or condition) to cleave the candidate linking group. It will also be desirable to also test the candidate cleavable linking group for the ability to resist cleavage in the blood or when in contact with other non-target tissue. Thus one can determine the relative susceptibility to cleavage between a first and a second condition, where the first is selected to be indicative of cleavage in a target cell and the second is selected to be indicative of cleavage in other tissues or biological fluids, e.g., blood or serum. The evaluations can be carried out in cell free systems, in cells, in cell culture, in organ or tissue culture, or in whole animals. It may be useful to make initial evaluations in cell-free or culture conditions and to confirm by further evaluations in whole animals. In preferred embodiments, useful candidate compounds are cleaved at least 2, 4, 10 or 100 times faster in the cell (or under in vitro conditions selected to mimic intracellular conditions) as compared to blood or serum (or under in vitro conditions selected to mimic extracellular conditions).

One class of cleavable linking groups is redox cleavable linking groups, which may be used in the present invention that are cleaved upon reduction or oxidation. An example of reductively cleavable linking group is a disulfide linking group (—S—S—). To determine if a candidate cleavable linking group is a suitable “reductively cleavable linking group,” or for example is suitable for use with a particular iRNA moiety and particular targeting agent one can look to methods described herein. For example, a candidate can be evaluated by incubation with dithiothreitol (DTT), or other reducing agent using reagents know in the art, which mimic the rate of cleavage which would be observed in a cell, e.g., a target cell. The candidates can also be evaluated under conditions which are selected to mimic blood or serum conditions. In a preferred embodiment, candidate compounds are cleaved by at most 10% in the blood. In preferred embodiments, useful candidate compounds are degraded at least 2, 4, 10 or 100 times faster in the cell (or under in vitro conditions selected to mimic intracellular conditions) as compared to blood (or under in vitro conditions selected to mimic extracellular conditions). The rate of cleavage of candidate compounds can be determined using standard enzyme kinetics assays under conditions chosen to mimic intracellular media and compared to conditions chosen to mimic extracellular media.

Phosphate-based cleavable linking groups, which may be used in the present invention, are cleaved by agents that degrade or hydrolyze the phosphate group. An example of an agent that cleaves phosphate groups in cells are enzymes such as phosphatases in cells. Examples of phosphate-based linking groups are —O—P(O)(ORk)-O—, —O—P(S)(ORk)-O—, —O—P(S)(SRk)-O—, —S—P(O)(ORk)-O—, —O—P(O)(ORk)-S—, —S—P(O)(ORk)-S—, —O—P(S)(ORk)-S—, —S—P(S)(ORk)-O—, —O—P(O)(Rk)-O—, —O—P(S)(Rk)-O—, —S—P(O)(Rk)-O—, —S—P(S)(Rk)-O—, —S—P(O)(Rk)-S—, —O—P(S)(Rk)-S—, wherein Rk at each occurrence can be, independently, hydrogen, C1-C20 alkyl, C1-C20 haloalkyl, C6-C10 aryl, C7-C12 aralkyl. Preferred embodiments are —O—P(O)(OH)—O—, —O—P(S)(OH)—O—, —O—P(S)(SH)—O—, —S—P(O)(OH)—O—, —O—P(O)(OH)—S—, —S—P(O)(OH)—S—, —O—P(S)(OH)—S—, —S—P(S)(OH)—O—, —O—P(O)(H)—O—, —O—P(S)(H)—O—, —S—P(O)(H)—O—, —S—P(S)(H)—O—, —S—P(O)(H)—S—, —O—P(S)(H)—S—. A preferred embodiment is —O—P(O)(OH)—O—. These candidates can be evaluated using methods analogous to those described above.

Acid cleavable linking groups, which may be used according to the present invention, are linking groups that are cleaved under acidic conditions. In preferred embodiments acid cleavable linking groups are cleaved in an acidic environment with a pH of about 6.5 or lower (e.g., about 6.0, 5.5, 5.0, or lower), or by agents such as enzymes that can act as a general acid. In a cell, specific low pH organelles, such as endosomes and lysosomes can provide a cleaving environment for acid cleavable linking groups. Examples of acid cleavable linking groups include but are not limited to hydrazones, esters, and esters of amino acids. Acid cleavable groups can have the general formula —C═NN—, C(O)O, or —OC(O). A preferred embodiment is when the carbon attached to the oxygen of the ester (the alkoxy group) is an aryl group, substituted alkyl group, or tertiary alkyl group such as dimethyl pentyl or t-butyl. These candidates can be evaluated using methods analogous to those described above.

Ester-based cleavable linking groups, which may be used in the present invention, are cleaved by enzymes such as esterases and amidases in cells. Examples of ester-based cleavable linking groups include but are not limited to esters of alkylene, alkenylene and alkynylene groups. Ester cleavable linking groups have the general formula —C(O)O—, or —OC(O)—. These candidates can be evaluated using methods analogous to those described above.

Peptide-based cleavable linking groups, which may be used in the present invention, are cleaved by enzymes such as peptidases and proteases in cells. Peptide-based cleavable linking groups are peptide bonds formed between amino acids to yield oligopeptides (e.g., dipeptides, tripeptides etc.) and polypeptides. Peptide-based cleavable groups do not include the amide group (—C(O)NH—). The amide group can be formed between any alkylene, alkenylene or alkynylene. A peptide bond is a special type of amide bond formed between amino acids to yield peptides and proteins. The peptide based cleavage group is generally limited to the peptide bond (i.e., the amide bond) formed between amino acids yielding peptides and proteins and does not include the entire amide functional group. Peptide-based cleavable linking groups have the general formula —NHCHR^{A}C(O)NHCHR^{B}C(O)—, where R^{A} and R^{B} are the R groups of the two adjacent amino acids.

In some embodiments of any one of the aspects described herein, L is an optionally substituted C₁-C₂₀alkylene, (e.g., —(CH₂)_{b}—, where b is 1, 2, 3, 4, 5, 6, 7, 8, 9, 10, 11, 12, 13, 14, 14, 15, 16, 17, 18, 19 or 20), or optionally substituted C₂-C₂₀alkynylene, and where the backbone of the alkylene or alkynylene can be interrupted or terminated by one or more of O, S, S(O), SO₂, NR^{N1}, NR^{N1}—C(O), C(O), C(O)O, cleavable linking group, substituted or unsubstituted aryl, substituted or unsubstituted heteroaryl, substituted or unsubstituted heterocyclic; where R^{N1} is hydrogen, acyl, aliphatic or substituted aliphatic.

In some embodiments of any one of the aspects, L is an optionally substituted C₁-C₂₀alkylene, where the backbone of the alkylene is interrupted and/or terminated with a NHC(O). For example, L is an optionally substituted C₅-C₁₅alkylene, where the backbone of the alkylene is interrupted and/or terminated with a NHC(O).

In some embodiments of any one of the aspects, L is —(CH₂)_{LM}—NHC(O)—(CH₂)_{LN}—, where LM and LN are independently 1, 2, 3, 4, 5, 6, 7, 8, 9, 10, 11 or 12. LM and LN can be the same or they can be different. In some embodiments, LM is 4, 5, 6, 7 or 8. In some embodiments, LN is 9, 10, 11 or 12.

### B (Nucleobase)

Embodiments of the any one of the aspects described herein include B, a modified or unmodified nucleobase.

It is noted that the nucleobase can be a natural nucleobase or a non-natural nucleobase. By a “non-natural nucleobase” is meant a nucleobase other than adenine, guanine, cytosine, uracil, or thymine. Exemplary non-natural nucleobases include, but are not limited to, inosine, xanthine, hypoxanthine, nubularine, isoguanisine, tubercidine, and substituted or modified analogs of adenine, guanine, cytosine and uracil, such as 2-aminoadenine and other alkyl derivatives of adenine and guanine, 2-propyl and other alkyl derivatives of adenine and guanine, 5-halouracil and cytosine, 5-propynyl uracil and cytosine, 6-azo uracil, cytosine and thymine, 5-uracil (pseudouracil), 4-thiouracil, 5-halouracil, 5-(2-aminopropyl)uracil, 5-amino allyl uracil, 8-halo, amino, thiol, thioalkyl, hydroxyl and other 8-substituted adenines and guanines, 5-trifluoromethyl and other 5-substituted uracils and cytosines, 7-methylguanine, 5-substituted pyrimidines, 6-azapyrimidines and N-2, N-6 and O-6 substituted purines, including 2-aminopropyladenine, 5-propynyluracil and 5-propynylcytosine, dihydrouracil, 3-deaza-5-azacytosine, 2-aminopurine, 5-alkyluracil, 7-alkylguanine, 5-alkyl cytosine, 7-deazaadenine, N6, N6-dimethyladenine, 2,6-diaminopurine, 5-amino-allyl-uracil, N3-methyluracil, substituted 1,2,4-triazoles, 2-pyridinone, 5-nitroindole, 3-nitropyrrole, 5-methoxyuracil, uracil-5-oxyacetic acid, 5-methoxycarbonylmethyluracil, 5-methyl-2-thiouracil, 5-methoxycarbonylmethyl-2-thiouracil, 5-methylaminomethyl-2-thiouracil, 3-(3-amino-3carboxypropyl)uracil, 3-methylcytosine, 5-methylcytosine, N⁴-acetyl cytosine, 2-thiocytosine, N6-methyladenine, N6-isopentyladenine, 2-methylthio-N6-isopentenyladenine, N-methylguanines, or O-alkylated bases. Further purines and pyrimidines include those disclosed in U.S. Pat. No. 3,687,808, those disclosed in the Concise Encyclopedia of Polymer Science and Engineering, pages 858-859, Kroschwitz, J. I., ed. John Wiley & Sons, 1990, and those disclosed by Englisch et al., Angewandte Chemie, International Edition, 1991, 30, 613, content of all which is incorporated herein by reference.

In some embodiments, the non-natural nucleobase can be selected from the group consisting of inosine, xanthine, hypoxanthine, nubularine, isoguanisine, tubercidine, 2-(halo)adenine, 2-(alkyl)adenine, 2-(propyl)adenine, 2-(amino)adenine, 2-(aminoalkyll)adenine, 2-(aminopropyl)adenine, 2-(methylthio)-N⁶-(isopentenyl)adenine, 7-(deaza)adenine, 8-(alkenyl)adenine, 8-(alkyl)adenine, 8-(alkynyl)adenine, 8-(amino)adenine, 8-(halo)adenine, 8-(hydroxyl)adenine, 8-(thioalkyl)adenine, 8-(thiol)adenine, N⁶-(isopentyl)adenine, N⁶-(methyl)adenine, N⁶, N⁶-(dimethyl)adenine, 2-(alkyl)guanine, 2-(propyl)guanine, 6-(alkyl)guanine, 6-(methyl)guanine, 7-(alkyl)guanine, 7-(methyl)guanine, 7-(deaza)guanine, 8-(alkyl)guanine, 8-(alkenyl)guanine, 8-(alkynyl)guanine, 8-(amino)guanine, 8-(halo)guanine, 8-(hydroxyl)guanine, 8-(thioalkyl)guanine, 8-(thiol)guanine, N-(methyl)guanine, 2-(thio)cytosine, 3-(deaza)-5-(aza)cytosine, 3-(alkyl)cytosine, 3-(methyl)cytosine, 5-(alkyl)cytosine, 5-(alkynyl)cytosine, 5-(halo)cytosine, 5-(methyl)cytosine, 5-(propynyl)cytosine, 5-(propynyl)cytosine, 5-(trifluoromethyl)cytosine, 6-(azo)cytosine, N⁴-(acetyl)cytosine, 3-(3-amino-3-carboxypropyl)uracil, 2-(thio)uracil, 5-(methyl)-2-(thio)uracil, 5-(methylaminomethyl)-2-(thio)uracil, 4-(thio)uracil, 5-(methyl)-4-(thio)uracil, 5-(methylaminomethyl)-4-(thio)uracil, 5-(methyl)-2,4-(dithio)uracil, 5-(methylaminomethyl)-2,4-(dithio)uracil, 5-(2-aminopropyl)uracil, 5-(alkyl)uracil, 5-(alkynyl)uracil, 5-(allylamino)uracil, 5-(aminoallyl)uracil, 5-(aminoalkyl)uracil, 5-(guanidiniumalkyl)uracil, 5-(1,3-diazole-1-alkyl)uracil, 5-(cyanoalkyl)uracil, 5-(dialkylaminoalkyl)uracil, 5-(dimethylaminoalkyl)uracil, 5-(halo)uracil, 5-(methoxy)uracil, uracil-5-oxyacetic acid, 5-(methoxycarbonylmethyl)-2-(thio)uracil, 5-(methoxycarbonyl-methyl)uracil, 5-(propynyl)uracil, 5-(propynyl)uracil, 5-(trifluoromethyl)uracil, 6-(azo)uracil, dihydrouracil, N³-(methyl)uracil, 5-uracil (i.e., pseudouracil), 2-(thio)pseudouracil, 4-(thio)pseudouracil, 2,4-(dithio)psuedouracil, 5-(alkyl)pseudouracil, 5-(methyl)pseudouracil, 5-(alkyl)-2-(thio)pseudouracil, 5-(methyl)-2-(thio)pseudouracil, 5-(alkyl)-4-(thio)pseudouracil, 5-(methyl)-4-(thio)pseudouracil, 5-(alkyl)-2,4-(dithio)pseudouracil, 5-(methyl)-2,4-(dithio)pseudouracil, 1-substituted pseudouracil, 1-substituted 2(thio)-pseudouracil, 1-substituted 4-(thio)pseudouracil, 1-substituted 2,4-(dithio)pseudouracil, 1-(aminocarbonylethylenyl)-pseudouracil, 1-(aminocarbonylethylenyl)-2(thio)-pseudouracil, 1-(aminocarbonylethylenyl)-4-(thio)pseudouracil, 1-(aminocarbonylethylenyl)-2,4-(dithio)pseudouracil, 1-(aminoalkylaminocarbonylethylenyl)-pseudouracil, 1-(aminoalkylamino-carbonylethylenyl)-2(thio)-pseudouracil, 1-(aminoalkylaminocarbonylethylenyl)-4-(thio)pseudouracil, 1-(aminoalkylaminocarbonylethylenyl)-2,4-(dithio)pseudouracil, 1,3-(diaza)-2-(oxo)-phenoxazin-1-yl, 1-(aza)-2-(thio)-3-(aza)-phenoxazin-1-yl, 1,3-(diaza)-2-(oxo)-phenthiazin-1-yl, 1-(aza)-2-(thio)-3-(aza)-phenthiazin-1-yl, 7-substituted 1,3-(diaza)-2-(oxo)-phenoxazin-1-yl, 7-substituted 1-(aza)-2-(thio)-3-(aza)-phenoxazin-1-yl, 7-substituted 1,3-(diaza)-2-(oxo)-phenthiazin-1-yl, 7-substituted 1-(aza)-2-(thio)-3-(aza)-phenthiazin-1-yl, 7-(aminoalkylhydroxy)-1,3-(diaza)-2-(oxo)-phenoxazin-1-yl, 7-(aminoalkylhydroxy)-1-(aza)-2-(thio)-3-(aza)-phenoxazin-1-yl, 7-(aminoalkylhydroxy)-1,3-(diaza)-2-(oxo)-phenthiazin-1-yl, 7-(aminoalkylhydroxy)-1-(aza)-2-(thio)-3-(aza)-phenthiazin-1-yl, 7-(guanidiniumalkylhydroxy)-1,3-(diaza)-2-(oxo)-phenoxazin-1-yl, 7-(guanidiniumalkylhydroxy)-1-(aza)-2-(thio)-3-(aza)-phenoxazin-1-yl, 7-(guanidiniumalkyl-hydroxy)-1,3-(diaza)-2-(oxo)-phenthiazin-1-yl, 7-(guanidiniumalkylhydroxy)-1-(aza)-2-(thio)-3-(aza)-phenthiazin-1-yl, 1,3,5-(triaza)-2,6-(dioxa)-naphthalene, inosine, xanthine, hypoxanthine, nubularine, tubercidine, isoguanisine, inosinyl, 2-aza-inosinyl, 7-deaza-inosinyl, nitroimidazolyl, nitropyrazolyl, nitrobenzimidazolyl, nitroindazolyl, aminoindolyl, pyrrolopyrimidinyl, 3-(methyl)isocarbostyrilyl, 5-(methyl)isocarbostyrilyl, 3-(methyl)-7-(propynyl)isocarbostyrilyl, 7-(aza)indolyl, 6-(methyl)-7-(aza)indolyl, imidizopyridinyl, 9-(methyl)-imidizopyridinyl, pyrrolopyrizinyl, isocarbostyrilyl, 7-(propynyl)isocarbostyrilyl, propynyl-7-(aza)indolyl, 2,4,5-(trimethyl)phenyl, 4-(methyl)indolyl, 4,6-(dimethyl)indolyl, phenyl, napthalenyl, anthracenyl, phenanthracenyl, pyrenyl, stilbenyl, tetracenyl, pentacenyl, difluorotolyl, 4-(fluoro)-6-(methyl)benzimidazole, 4-(methyl)benzimidazole, 6-(azo)thymine, 2-pyridinone, 5-nitroindole, 3-nitropyrrole, 6-(aza)pyrimidine, 2-(amino)purine, 2,6-(diamino)purine, 5-substituted pyrimidines, N²-substituted purines, N⁶-substituted purines, O⁶-substituted purines, substituted 1,2,4-triazoles, and any O-alkylated or N-alkylated derivatives thereof.

In some embodiments, a non-natural nucleobase is a modified nucleobase, i.e., the nucleobase comprises a nucleobase modification described herein, e.g., the nucleobase is a substituted or modified analog of any of the natural nucleobases. Examples of the nucleobase modifications include, but not limited to: C-5 pyrimidine with an alkyl group or aminoalkyls and other cationic groups such as guanidinium and amidine functionalities, N²— and N⁶— with an alkyl group or aminoalkyls and other cationic groups such as guanidinium and amidine functionalities of purines, G-clamps, guanidinium G-clamps, and pseudouridine known in the art.

In some embodiments of any one of the aspects, the non-natural nucleobase is a universal nucleobase. As used herein, a universal nucleobase is any modified or unmodified natural or non-natural nucleobase that can base pair with all of adenine, cytosine, guanine and uracil without substantially affecting the melting behavior, recognition by intracellular enzymes or activity of the oligonucleotide comprising the universal nucleobase. Some exemplary universal nucleobases include, but are not limited to, 2,4-difluorotoluene, nitropyrrolyl, nitroindolyl, 8-aza-7-deazaadenine, 4-fluoro-6-methylbenzimidazle, 4-methylbenzimidazle, 3-methyl isocarbostyrilyl, 5-methyl isocarbostyrilyl, 3-methyl-7-propynyl isocarbostyrilyl, 7-azaindolyl, 6-methyl-7-azaindolyl, imidizopyridinyl, 9-methyl-imidizopyridinyl, pyrrolopyrizinyl, isocarbostyrilyl, 7-propynyl isocarbostyrilyl, propynyl-7-azaindolyl, 2,4,5-trimethylphenyl, 4-methylinolyl, 4,6-dimethylindolyl, phenyl, napthalenyl, anthracenyl, phenanthracenyl, pyrenyl, stilbenyl, tetracenyl, pentacenyl, and structural derivatives thereof.

In some embodiments of any one of the aspects described herein, the non-natural nucleobase is a protected nucleobase. As used herein, a “protected nucleobase” refers to a nucleobase comprising a nitrogen protecting group, and/or an oxygen protecting group, and/or a sulfur protecting group.

In some embodiments of any one of the aspects described herein, the non-natural nucleobase is a modified, protected or substituted analogs of a nucleobase selected from adenine, cytosine, guanine, thymine, and uracil.

### Oxygen Protecting Groups

Some embodiments of the any one of the aspects described herein include an oxygen protecting group (also referred to as an hydroxyl protecting group herein). Oxygen protecting groups include, but are not limited to, —R^{OP1}, —N(R^{OP2})₂, —C(═O)SR^{OP1}, —C(═O)R^{OP1}, —CO₂R^{OP1}, —C(═O)N(R^{OP2})₂, —C(═NR^{OP2})R^{OP1}, —C(═NR^{OP2})OR^{OP1}, —C(═NR^{OP2})N(Ror²)₂, —S(═O)RoP¹, —SO⁺₂R^{OP1}, —Si(R^{OP1})₃, —P(R^{OP3})₂, —P(R^{OP3})⁺₃X⁻, —P(OR^{OP3})₂, —P(OR^{OP3})₃X⁻, —P(═O)(R^{OP1})₂, —P(═O)(OR^{OP3})₂, and —P(═O)(N(R^{OP2})₂)₂; wherein each X is a counterion; each R^{OP1} is independently C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀alkenyl, heteroC₂₋₁₀alkynyl, C₃₋₁₀ carbocyclyl, 3-14 membered heterocyclyl, C₆₋₁₄ aryl, or 5-14 membered heteroaryl, or two R^{OP1} groups are joined to form a 3-14 membered heterocyclyl or 5-14 membered heteroaryl ring; each R^{OP2} is hydrogen, —OH, —OR^{OP1}, —N(R^{OP3})₂, —CN, —C(═O)R^{OP1}, —C(═O)N(R^{OP3})₂, —CO₂R^{OP1}, —SO₂R^{OP1}, —C(═NR^{OP3})OR^{OP1}, —C(═NR^{OP3})N(R^{OP3})₂, —SO₂N(R^{OP3})₂, —SO₂R^{OP3}, —SO₂OR^{OP3}, —SOR^{OP1}, —C(═S)N(R^{OP3})₂, —C(═O)SR^{OP3}, —C(═S)SR^{OP3}, —P(═O)(R^{OP1})₂, —P(═O)(OR^{OP3})₂, —P(═O)(N(R^{OP3})₂)₂, C₁₋₁₀ alkyl, C₁₋₁₀ perhaloalkyl, C₂₋₁₀ alkenyl, C₂₋₁₀ alkynyl, heteroC₁₋₁₀alkyl, heteroC₂₋₁₀alkenyl, heteroC₂₋₁₀ alkynyl, C₃₋₁₀ carbocyclyl, 3-14 membered heterocyclyl, C₆₋₁₄ aryl, and 5-14 membered heteroaryl, or two R^{OP2} groups are joined to form a 3-14 membered heterocyclyl or 5-14 membered heteroaryl ring; and each R^{OP3} is independently hydrogen, C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, or two R^{OP3} groups are joined to form a 3-14 membered heterocyclyl or 5-14 membered heteroaryl ring; and wherein each alkyl, alkenyl, alkynyl, carbocyclyl, heterocyclyl, aralkyl, aryl, and heteroaryl of R^{OP1}, R^{OP2} and R^{OP3} can be optionally substituted with 1, 2, 3, 4 or 5 substituents independently selected from OH, CN, SC(O)Ph, oxo (═O), SH, SO₂NH₂, SO₂(C₁-C₄)alkyl, SO₂NH(C₁-C₄)alkyl, halogen, carbonyl, thiol, cyano, NH₂, NH(C₁-C₄)alkyl, N[(C₁-C₄)alkyl]₂, C(O)NH₂, COOH, COOMe, acetyl, (C₁-C₈)alkyl, O(C₁-C₈)alkyl (i.e., C₁-C₈alkoxy), O(C₁-C₈)haloalkyl, (C₂-C₈)alkenyl, (C₂-C₈)alkynyl, haloalkyl, thioalkyl, cyanomethylene, alkylaminyl, aryl, heteroaryl, substituted aryl, NH₂—C(O)-alkylene, NH(Me)-C(O)-alkylene, CH₂—C(O)-alkyl, C(O)-alkyl, alkylcarbonylaminyl, CH₂—[CH(OH)]ₘ—(CH₂)ₚ—OH, CH₂—[CH(OH)]ₘ—(CH₂)ₚ—NH₂ or CH₂-aryl-alkoxy, where “m” and “p” are independently 1, 2, 3, 4, 5 or 6.

Oxygen protecting groups are well known in the art and include those described in detail in Greene's Protecting Groups in Organic Synthesis, P. G. M. Wuts, 5^{th} Edition, John Wiley & Sons, 2014, incorporated herein by reference.

Exemplary oxygen protecting groups include, but are not limited to, methyl, t-butyloxycarbonyl (BOC or Boc), methoxylmethyl (MOM), methylthiomethyl (MTM), t-butylthiomethyl, (phenyldimethylsilyl)methoxymethyl (SMOM), benzyloxymethyl (BOM), p-methoxybenzyloxymethyl (PMBM), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl)ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl (Bn), p-methoxybenzyl, 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, u-naphthyldiphenylmethyl, p-methoxyphenyldiphenylmethyl, di(p-methoxyphenyl)phenylmethyl, tri(p-methoxyphenyl)methyl, 4-(4′-bromophenacyloxyphenyl)diphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 3-(imidazol-1-yl)bis(4′,4″-dimethoxyphenyl)methyl, 1,1-bis(4-methoxyphenyl)-1′-pyrenylmethyl, 9-anthryl, 9-(9-phenyl)xanthenyl, 9-(9-phenyl-10-oxo)anthryl, 1,3-benzodisulfuran-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio)pentanoate (levulinoyldithioacetal), adamantoate, crotonate, 4-methoxycrotonate, benzoate, p-phenylbenzoate, 2,4,6-trimethylbenzoate (mesitoate), alkyl methyl carbonate, 9-fluorenylmethyl carbonate (Fmoc), alkyl ethyl carbonate, alkyl 2,2,2-trichloroethyl carbonate (Troc), 2-(trimethylsilyl)ethyl carbonate (TMSEC), 2-(phenylsulfonyl) ethyl carbonate (Psec), 2-(triphenylphosphonio) ethyl carbonate (Peoc), alkyl isobutyl carbonate, alkyl vinyl carbonate alkyl allyl carbonate, alkyl p-nitrophenyl carbonate, alkyl benzyl carbonate, alkyl p-methoxybenzyl carbonate, alkyl 3,4-dimethoxybenzyl carbonate, alkyl o-nitrobenzyl carbonate, alkyl p-nitrobenzyl carbonate, alkyl S-benzyl thiocarbonate, 4-ethoxy-1-napththyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl, 4-(methylthiomethoxy)butyrate, 2-(methylthiomethoxymethyl)benzoate, 2,6-dichloro-4-methylphenoxyacetate, 2,6-dichloro-4-(1,1,3,3-tetramethylbutyl)phenoxyacetate, 2,4-bis(1,1-dimethylpropyl)phenoxyacetate, chlorodiphenylacetate, isobutyrate, monosuSP3inoate, (E)-2-methyl-2-butenoate, o-(methoxyacyl)benzoate, u-naphthoate, nitrate, alkylN,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts).

In some embodiments of any one of the aspects described herein, oxygen protecting group is acetyl, benzyl, benzoyl, 2,6-dichlorobenzyl, t-butyldimethylsilyl (TBDMS), t-butyldiphenylsilyl, trimethylsilyl (TMS), triisopropylsilyl (TIPS), mesylate, tosylate, 4,4′-dimethoxytrityl (DMT), 9-phenylxanthine-9-yl (Pixyl) and 9-(p-methoxyphenyl)xanthine-9-yl (MOX). In certain embodiments, the hydroxyl protecting group is selected from acetyl, benzyl, t-butyldimethylsilyl, t-butyldiphenylsilyl, trimethylsilyl (TMS), triisopropylsilyl (TIPS), and dimethoxytrityl wherein a more preferred hydroxyl protecting group is 4,4′-dimethoxytrityl.

The terms “protected hydroxyl” and “protected hydroxyl” as used herein mean a group of the formula —OR^{Pro}, wherein R^{Pro} is an oxygen protecting group as defined herein.

### Nitrogen Protecting Groups

Some embodiments of the any one of the aspects described herein include a nitrogen protecting group (also referred to as an amino protecting group herein). Nitrogen protecting groups include, but are not limited to, —OH, —OR^{NP1}, —N(R^{NP2})₂, —C(═O)R^{NP1}, —C(═O)N(R^{NP2})₂, —CO₂R^{NP1}, —SO₂R^{NP1}, —C(═NR^{NP2})R^{NP1}, —C(═NR^{NP2})OR^{NP1}, —C(═NR^{NP2})N(R^{NP2})₂, —SO₂N(R^{NP2})₂, —SO₂R^{P2}, —SO₂OR^{NP2}, —SOR^{NP1}, —C(═S)N(R^{NP2})₂, —C(═O)SR^{NP2}, —C(═S)SR^{NP2}, C₁₋₁₀ alkyl (e.g., aralkyl, heteroaralkyl), C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl groups, where each R^{NP1} is independently C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀alkenyl, heteroC₂₋₁₀alkynyl, C₃₋₁₀ carbocyclyl, 3-14 membered heterocyclyl, C₆₋₁₄ aryl, or 5-14 membered heteroaryl, or two R^{NP1} groups are joined to form a 3-14 membered heterocyclyl or 5-14 membered heteroaryl ring; and each R^{NP2} is independently hydrogen, C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, or two R^{SP3} groups are joined to form a 3-14 membered heterocyclyl or 5-14 membered heteroaryl ring, and wherein each alkyl, alkenyl, alkynyl, carbocyclyl, heterocyclyl, aralkyl, aryl, and heteroaryl of R^{NP1} and R^{NP2} can be optionally substituted with 1, 2, 3, 4 or 5 substituents independently selected from OH, CN, SC(O)Ph, oxo (═O), SH, SO₂NH₂, SO₂(C₁-C₄)alkyl, SO₂NH(C₁-C₄)alkyl, halogen, carbonyl, thiol, cyano, NH₂, NH(C₁-C₄)alkyl, N[(C₁-C₄)alkyl]₂, C(O)NH₂, COOH, COOMe, acetyl, (C₁-C₈)alkyl, O(C₁-C₈)alkyl (i.e., C₁-C₈alkoxy), O(C₁-C₈)haloalkyl, (C₂-C₈)alkenyl, (C₂-C₈)alkynyl, haloalkyl, thioalkyl, cyanomethylene, alkylaminyl, aryl, heteroaryl, substituted aryl, NH₂—C(O)-alkylene, NH(Me)-C(O)-alkylene, CH₂—C(O)-alkyl, C(O)-alkyl, alkylcarbonylaminyl, CH₂—[CH(OH)]ₘ—(CH₂)ₚ—OH, CH₂—[CH(OH)]ₘ—(CH₂)ₚ—NH₂ or CH₂-aryl-alkoxy, where “m” and “p” are independently 1, 2, 3, 4, 5 or 6.

Nitrogen protecting groups are well known in the art and include those described in detail in Greene's Protecting Groups in Organic Synthesis, P. G. M. Wuts, 5^{th} Edition, John Wiley & Sons, 2014, incorporated herein by reference.

Exemplary amide (e.g., —C(═O)R^{NP1}) nitrogen protecting groups include, but are not limited to, formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivative, benzamide, p-phenylbenzamide, o-nitophenylacetamide, o-nitrophenoxyacetamide, acetoacetamide, (N′-dithiobenzyloxy acylamino)acetamide, 3-(p-hydroxylphenyl)propanamide, 3-(o-nitrophenyl)propanamide, 2-methyl-2-(o-nitrophenoxy)propanamide, 2-methyl-2-(o-phenylazophenoxy)propanamide, 4-chlorobutanamide, 3-methyl-3-nitrobutanamide, o-nitrocinnamide, N-acetylmethionine derivative, o-nitrobenzamide, and o-(benzoyloxymethyl)benzamide.

Exemplary carbamate (e.g., —C(═O)OR^{NP1}) nitrogen protecting groups include, but are not limited to, methyl carbamate, ethyl carbamate, 9-fluorenylmethyl carbamate (Fmoc), 9-(2-sulfo)fluorenylmethyl carbamate, 9-(2,7-dibromo)fluoroenylmethyl carbamate, 2,7-di-t-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, t-butyl carbamate (BOC or Boc), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxylpiperidinyl carbamate, alkyldithio carbamate, benzyl carbamate (Cbz), p-methoxybenzyl carbamate (Moz), p-nitobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxylboryl)benzyl carbamate, 5-benzisoxazolylmethyl carbamate, 2-(trifluoromethyl)-6-chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl(o-nitrophenyl)methyl carbamate, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxyacylvinyl carbamate, o-(N,N-dimethylcarboxamido)benzyl carbamate, 1,1-dimethyl-3-(N,N-dimethylcarboxamido)propyl carbamate, 1,1-dimethylpropynyl carbamate, di(2-pyridyl)methyl carbamate, 2-furanylmethyl carbamate, 2-iodoethyl carbamate, isoborynl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, and 2,4,6-trimethylbenzyl carbamate.

Exemplary sulfonamide (e.g., —S(═O)₂R^{NP1}) nitrogen protecting groups include, but are not limited to, such as p-toluenesulfonamide (Ts), benzenesulfonamide, 2,3,6, -trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), β-trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, and phenacylsulfonamide.

Additional exemplary nitrogen protecting groups include, but are not limited to, phenothiazinyl-(10)-acyl derivative, N′-p-toluenesulfonylaminoacyl derivative, N′-phenylaminothioacyl derivative, N-benzoylphenylalanyl derivative, N-acetylmethionine derivative, 4,5-diphenyl-3-oxazolin-2-one, N-phthalimide, N-dithiasuNP2inimide (Dts), N—2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethylsilyl)ethoxy]methylamine (SEM), N-3-acetoxypropylamine, N-(1-isopropyl-4-nitro-2-oxo-3-pyroolin-3-yl)amine, quaternary ammonium salts, N-benzylamine, N-di(4-methoxyphenyl)methylamine, N-5-dibenzosuberylamine, N-triphenylmethylamine (Tr), N-[(4-methoxyphenyl)diphenylmethyl]amine (MMTr), N-9-phenylfluorenylamine (PhF), N-2,7-dichloro-9-fluorenylmethyleneamine, N-ferrocenylmethylamino (Fcm), N-2-picolylamino N′-oxide, N-1,1-dimethylthiomethyleneamine, N-benzylideneamine, N-p-methoxybenzylideneamine, N-diphenylmethyleneamine, N-[(2-pyridyl)mesityl]methyleneamine, N—(N′,N′-dimethylaminomethylene)amine, N,N′-isopropylidenediamine, N-p-nitrobenzylideneamine, N-salicylideneamine, N—S-chlorosalicylideneamine, N-(5-chloro-2-hydroxylphenyl)phenylmethyleneamine, N-cyclohexylideneamine, N-(5,5-dimethyl-3-oxo-1-cyclohexenyl)amine, N-borane and N-diphenylborinic acid derivative, N-[phenyl(pentNP1cylchromium- or tungsten)acyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxybenzenesulfenamide, triphenylmethylsulfenamide, and 3-nitropyridinesulfenamide (Npys).

### Sulfur Protecting Groups

Some embodiments of the any one of the aspects described herein include sulfur protecting group (also referred to as a thiol protecting group herein). Sulfur protecting groups include, but are not limited to, —R^{SP1}, —N(R^{SP2})₂, —C(═O)SR^{SP1}, —C(═O)R^{SP1}—COR^{SP1}, —C(═O)N(R^{SP2})₂, —C(═NR^{SP2})R^{SP1}, —C(═NR^{SP2})OR^{SP1}, —C(═NR^{SP2})N(R^{SP2})₂, —S(═O)R^{SP1}, —SO₂R^{SP1}, —Si(R^{SP1})₃, —P(R^{SP3})₂, —P(R^{SP3})+₃X, —P(OR^{SP3})₂, —P(OR^{SP3})+₃X, —P(═O)(R^{SP1})₂, —P(═O)(OR^{SP3})₂, and —P(═O)(N(R^{SP2})₂)₂, wherein

X⁻ is a counterion; each R^{SP1} is independently C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀alkenyl, heteroC₂₋₁₀alkynyl, C₃₋₁₀ carbocyclyl, 3-14 membered heterocyclyl, C₆₋₁₄ aryl, or 5-14 membered heteroaryl, or two R^{SP1} groups are joined to form a 3-14 membered heterocyclyl or 5-14 membered heteroaryl ring; each R^{SP2} is hydrogen, —OH, —OR^{SP1}, —N(R^{SP3})₂, —CN, —C(═O)R^{SP1}, —C(═O)N(R^{SP3})₂, —CO₂R^{SP1}, —SO₂R^{SP1}, —C(═NR^{SP3})OR^{SP1}, —C(═NR^{SP3})N(R^{SP3})₂, —SO₂N(R^{SP3})₂, —SO₂R^{SP3}, —SO₂OR^{SP3}, —SOR^{SP1}, —C(═S)N(R^{SP3})₂, —C(═O)SR^{SP3}, —C(═S)SR^{SP3}, —P(═O)(R^{SP1})₂, —P(═O)(OR^{SP3})₂, —P(═O)(N(R^{SP3})₂)₂, C₁₋₁₀ alkyl, C₁₋₁₀ perhaloalkyl, C₂₋₁₀ alkenyl, C₂₋₁₀ alkynyl, heteroC₁₋₁₀alkyl, heteroC₂₋₁₀alkenyl, heteroC₂₋₁₀alkynyl, C₃₋₁₀ carbocyclyl, 3-14 membered heterocyclyl, C₆₋₁₄ aryl, and 5-14 membered heteroaryl, or two R^{SP2} groups are joined to form a 3-14 membered heterocyclyl or 5-14 membered heteroaryl ring; and each R^{SP3} is independently hydrogen, C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, or two R^{SP3} groups are joined to form a 3-14 membered heterocyclyl or 5-14 membered heteroaryl ring; and wherein each alkyl, alkenyl, alkynyl, carbocyclyl, heterocyclyl, aralkyl, aryl, and heteroaryl of R^{SP1}, R^{SP2} and R^{SP3} can be optionally substituted with 1, 2, 3, 4 or 5 substituents independently selected from OH, CN, SC(O)Ph, oxo (═O), SH, SO₂NH₂, SO₂(C₁-C₄)alkyl, SO₂NH(C₁-C₄)alkyl, halogen, carbonyl, thiol, cyano, NH₂, NH(C₁-C₄)alkyl, N[(C₁-C₄)alkyl]₂, C(O)NH₂, COOH, COOMe, acetyl, (C₁-C₈)alkyl, O(C₁-C₈)alkyl (i.e., C₁-C₈alkoxy), O(C₁-C₈)haloalkyl, (C₂-C₈)alkenyl, (C₂-C₈)alkynyl, haloalkyl, thioalkyl, cyanomethylene, alkylaminyl, aryl, heteroaryl, substituted aryl, NH₂—C(O)-alkylene, NH(Me)-C(O)-alkylene, CH₂—C(O)-alkyl, C(O)-alkyl, alkylcarbonylaminyl, CH₂—[CH(OH)]ₘ—(CH₂)ₚ—OH, CH₂—[CH(OH)]ₘ—(CH₂)ₚ—NH₂ or CH₂-aryl-alkoxy, where “m” and “p” are independently 1, 2, 3, 4, 5 or 6

Sulfur protecting groups are well known in the art and include those described in detail in Greene's Protecting Groups in Organic Synthesis, P. G. M. Wuts, 5^{th} Edition, John Wiley & Sons, 2014, incorporated herein by reference.

### Internucleoside Linkages

As used herein, “internucleoside linkage” refers to a covalent linkage between adjacent nucleosides. The two main classes of internucleoside linkages are defined by the presence or absence of a phosphorus atom. Representative phosphorus containing linkages include, but are not limited to, phosphodiesters (P=O), phosphotriesters, methylphosphonates, phosphoramidate, and phosphorothioates (P=S). Representative non-phosphorus containing linking groups include, but are not limited to, methylenemethylimino (—CH₂—N(CH₃)—O—CH₂—), thiodiester (—O—C(O) S—), thionocarbamate (—O—C(O)(NH)—S—); siloxane (—O—Si(H)₂-O—); and N,N′-dimethylhydrazine (—CH₂—N(CH₃)—N(CH₃)—). Modified internucleoside linkages, compared to natural phosphodiester linkages, can be used to alter, typically increase, nuclease resistance of the oligonucleotide compound. In certain embodiments, linkages having a chiral atom can be prepared as racemic mixtures, as separate enantiomers. Representative chiral linkages include, but are not limited to, alkylphosphonates and phosphorothioates. Methods of preparation of phosphorous-containing and non-phosphorous-containing linkages are well known to those skilled in the art.

The phosphate group in the internucleoside linkage can be modified by replacing one of the oxygens with a different substituent. One result of this modification can be increased resistance of the oligonucleotide to nucleolytic breakdown. Examples of modified phosphate groups include phosphorothioate, phosphoroselenates, borano phosphates, borano phosphate esters, hydrogen phosphonates, phosphoroamidates, alkyl or aryl phosphonates and phosphotriesters. In some embodiments, one of the non-bridging phosphate oxygen atoms in the phosphodiester internucleoside linkage can be replaced by any of the following: S, Se, BR₃ (R is hydrogen, alkyl, aryl), C (i.e. an alkyl group, an aryl group, etc . . . ), H, NR₂ (R is hydrogen, optionally substituted alkyl, aryl), or OR (R is optionally substituted alkyl or aryl). The phosphorous atom in an unmodified phosphate group is achiral. However, replacement of one of the non-bridging oxygens with one of the above atoms or groups of atoms renders the phosphorous atom chiral. In other words a phosphorous atom in a phosphate group modified in this way is a stereogenic center. The stereogenic phosphorous atom can possess either the “R” configuration (herein Rp) or the “S” configuration (herein Sp).

Phosphorodithioates have both non-bridging oxygens replaced by sulfur. The phosphorus center in the phosphorodithioates is achiral which precludes the formation of oligonucleotides diastereomers. Thus, while not wishing to be bound by theory, modifications to both non-bridging oxygens, which eliminate the chiral center, e.g. phosphorodithioate formation, can be desirable in that they cannot produce diastereomer mixtures. The non-bridging oxygens can be independently any one of O, S, Se, B, C, H, N, or OR (R is alkyl or aryl).

A phosphodiester internucleoside linkage can also be modified by replacement of bridging oxygen, (i.e. oxygen that links the phosphate to the sugar of the nucleosides), with nitrogen (bridged phosphoroamidates), sulfur (bridged phosphorothioates) and carbon (bridged methylenephosphonates). The replacement can occur at the either one of the linking oxygens or at both linking oxygens. When the bridging oxygen is the 3′-oxygen of a nucleoside, replacement with carbon is preferred. When the bridging oxygen is the 5′-oxygen of a nucleoside, replacement with nitrogen is preferred.

Modified phosphate linkages where at least one of the oxygen linked to the phosphate has been replaced or the phosphate group has been replaced by a non-phosphorous group, are also referred to as “non-phosphodiester intersugar linkage” or “non-phosphodiester linker.”

In certain embodiments, the phosphate group can be replaced by non-phosphorus containing connectors, e.g. dephospho linkers. Dephospho linkers are also referred to as non-phosphodiester linkers herein. While not wishing to be bound by theory, it is believed that since the charged phosphodiester group is the reaction center in nucleolytic degradation, its replacement with neutral structural mimics should impart enhanced nuclease stability. Again, while not wishing to be bound by theory, it can be desirable, in some embodiment, to introduce alterations in which the charged phosphate group is replaced by a neutral moiety.

Examples of moieties which can replace the phosphate group include, but are not limited to, amides (for example amide-3 (3′-CH₂—C(═O)—N(H)-5′) and amide-4 (3′-CH₂—N(H)—C(═O)-5′)), hydroxylamino, siloxane (dialkylsiloxane), carboxamide, carbonate, carboxymethyl, carbamate, carboxylate ester, thioether, ethylene oxide linker, sulfide, sulfonate, sulfonamide, sulfonate ester, thioformacetal (3′-S—CH₂—O-5′), formacetal (3′-O—CH₂—O-5′), oxime, methyleneimino, methylenecarbonylamino, methylenemethylimino (MMI, 3′-CH₂—N(CH₃)—O-5′), methylenehydrazo, methylenedimethylhydrazo, methyleneoxymethylimino, ethers (C3′-O-C5′), thioethers (C3′-S-C5′), thioacetamido (C3′-N(H)—C(═O)—CH₂—S-C5′, C3′-O—P(O)—O—SS-C5′, C3′-CH₂—NH—NH-C5′, 3′—NHP(O)(OCH₃)—O-5′ and 3′—NHP(O)(OCH₃)—O-5′ and nonionic linkages containing mixed N, O, S and CH₂ component parts. See for example, Carbohydrate Modifications in Antisense Research; Y. S. Sanghvi and P. D. Cook Eds. ACS Symposium Series 580; Chapters 3 and 4, (pp. 40-65). Preferred embodiments include methylenemethylimino (MMI), methylenecarbonylamino, amides, carbamate and ethylene oxide linker.

One skilled in the art is well aware that in certain instances replacement of a non-bridging oxygen can lead to enhanced cleavage of the intersugar linkage by the neighboring 2′-OH, thus in many instances, a modification of a non-bridging oxygen can necessitate modification of 2′-OH, e.g., a modification that does not participate in cleavage of the neighboring intersugar linkage, e.g., arabinose sugar, 2′-O-alkyl, 2′-F, LNA and ENA.

Preferred non-phosphodiester internucleoside linkages include phosphorothioates, phosphorothioates with an at least 1%, 5%, 10%, 20%, 30%, 40%, 50%, 60%, 70%, 80%, 90% 95% or more enantiomeric excess of Sp isomer, phosphorothioates with an at least 1%, 5%, 10%, 20%, 30%, 40%, 50%, 60%, 70%, 80%, 90% 95% or more enantiomeric excess of Rp isomer, phosphorodithioates, phsophotriesters, aminoalkylphosphotrioesters, alkyl-phosphonaters (e.g., methyl-phosphonate), selenophosphates, phosphoramidates (e.g., N-alkylphosphoramidate), and boranophosphonates.

Additional exemplary non-phosphorus containing internucleoside linking groups are described in U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; 5,792,608; 5,646,269 and 5,677,439, content of each of which is incorporated herein by reference.

In some embodiments of any one of the aspects, the oligonucleotides described herein comprise one or more neutral internucleoside linkages that are non-ionic. Suitable neutral internucleoside linkages include, but are not limited to, phosphotriesters, methylphosphonates, MMI (3′-CH₂—N(CH₃)—O-5′), amide-3 (3′-CH₂—C(═O)—N(H)-5′), amide-4 (3′-CH₂—N(H)—C(═O)-5′), formacetal (3′-O—CH₂—O-5′), and thioformacetal (3′-S—CH₂—O-5′); nonionic linkages containing siloxane (dialkylsiloxane), carboxylate ester, carboxamide, sulfide, sulfonate ester and/or amides (See for example: Carbohydrate Modifications in Antisense Research; Y. S. Sanghvi and P. D. Cook Eds. ACS Symposium Series 580; Chapters 3 and 4, (pp. 40-65)); and nonionic linkages containing mixed N, O, S and CH₂ component parts.

In one embodiment, the non-phosphodiester backbone linkage is include, for example, phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′-linked analogs of these, and those having inverted polarity wherein the adjacent pairs of nucleoside units are linked 3′-5′ to 5′-3′ or 2′-5′ to 5′-2′.

Various salts, mixed salts and free acid forms are also included. In some embodiments of the invention, the dsRNA agents of the invention are in a free acid form. In other embodiments of the invention, the dsRNA agents of the invention are in a salt form. In one embodiment, the dsRNA agents of the invention are in a sodium salt form. In certain embodiments, when the dsRNA agents of the invention are in the sodium salt form, sodium ions are present in the agent as counterions for substantially all of the phosphodiester and/or phosphorothioate groups present in the agent. Agents in which substantially all of the phosphodiester and/or phosphorothioate linkages have a sodium counterion include not more than 5, 4, 3, 2, or 1 phosphodiester and/or phosphorothioate linkages without a sodium counterion. In some embodiments, when the dsRNA agents of the invention are in the sodium salt form, sodium ions are present in the agent as counterions for all of the phosphodiester and/or phosphorothioate groups present in the agent.

Representative U.S. patents that teach the preparation of the above phosphorus-containing linkages include, but are not limited to, U.S. Pat. Nos. 3,687,808; 4,469,863; 4,476,301; 5,023,243; 5,177,195; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,316; 5,550,111; 5,563,253; 5,571,799; 5,587,361; 5,625,050; 6,028,188; 6,124,445; 6,160,109; 6,169,170; 6,172,209; 6,239,265; 6,277,603; 6,326,199; 6,346,614; 6,444,423; 6,531,590; 6,534,639; 6,608,035; 6,683,167; 6,858,715; 6,867,294; 6,878,805; 7,015,315; 7,041,816; 7,273,933; 7,321,029; and U.S. Pat. RE39464, the entire contents of each of which are hereby incorporated herein by reference.

In some embodiments of any one of the aspects described herein, both of R²³ and R²⁵ are a bond to a modified internucleoside linkage.

In some embodiments of any one of the aspects described herein R²³ is a bond to phosphodiester internucleoside linkage.

In some embodiments of any one of the aspects described herein R²⁵ is a bond to phosphodiester internucleoside linkage.

In some embodiments of any one of the aspects described herein, R²³ is a bond to a modified internucleoside linkage and R²⁵ is a bond to phosphodiester internucleoside linkage.

In some embodiments of any one of the aspects described herein, R⁵ is a bond to a modified internucleoside linkage and R²³ is a bond to phosphodiester internucleoside linkage.

In some embodiments of any one of the aspects, the oligonucleotide can comprise one or more, e.g., 1, 2, 3, 4, 5, 6, 7, 8 or more modified internucleoside linkages. For example, the oligonucleotide can comprise 1, 2, 3, 4, 5 or 6 (e.g., 1, 2, 3 or 4) modified internucleoside linkages. In some embodiments, the oligonucleotide comprises at least two modified internucleoside linkages between the first five nucleotides counting from the 5′-end of the oligonucleotide and further comprises at least two modified internucleoside linkages between the first five nucleotides counting from the 3′-end of the oligonucleotide. For example, the oligonucleotide comprises modified internucleoside linkages between nucleotides 1 and 2, and between nucleotides 2 and 3, counting from 5′-end of the oligonucleotide, and between nucleotides 1 and 2, and between nucleotides 2 and 3, counting from 3′-end of the oligonucleotide.

In some embodiments of any one of the aspects, the modified internucleoside linkage is a phosphorothioate. Accordingly, in some embodiments of any one of the aspects, the oligonucleotide comprises one or more, e.g., 1, 2, 3, 4, 5, 6, 7, 8 or more phosphorothioate internucleoside linkages. For example, the oligonucleotide comprises 1, 2, 3, 4, 5 or 6 (e.g., 1, 2, 3, or 4) phosphorothioate internucleoside linkages. In some embodiments, the oligonucleotide comprises at least two phosphorothioate internucleoside linkages between the first five nucleotides counting from the 5′-end of the oligonucleotide and further comprises at least two phosphorothioate internucleoside linkages between the first five nucleotides counting from the 3′-end of the oligonucleotide. For example, the oligonucleotide comprises modified internucleoside linkages between nucleotides 1 and 2, and between nucleotides 2 and 3, counting from 5′-end of the oligonucleotide, and between nucleotides 1 and 2, and between nucleotides 2 and 3, counting from 3′-end of the oligonucleotide.

In some embodiments, the oligonucleotide comprises 1-10 blocks of two to ten phosphorothioate or methylphosphonate internucleotide linkages separated by 1, 2, 3, 4, 5, 6, 7, 8, 9, 10, 11, 12, 13, 14, 15 or 16 phosphate internucleotide linkages. For example, oligonucleotide comprises 2, 3, 4, 5, 6, 7, 8, or 9 blocks of two phosphorothioate or methylphosphonate internucleotide linkages separated by 1, 2, 3, 4, 5, 6, 7, 8, 9, 10, 11, 12, 13, 14, 15, 16, 17, or 18 phosphate internucleotide linkages.

In some embodiments of any one of the aspects described herein, the oligonucleotide comprises a pattern of backbone chiral centers. In some embodiments, a common pattern of backbone chiral centers comprises 5, 6, 7, 8, 9, 10, 11, 12, 13, 14, 15, 16, 17, 18 or more internucleotidic linkages in the Sp configuration. In some embodiments, a common pattern of backbone chiral centers comprises no more than 1, 2, 3, 4, 5, 6, 7 or 8 internucleotidic linkages in the Rp configuration. In some embodiments, a common pattern of backbone chiral centers comprises no more than 1, 2, 3, 4, 5, 6, 7 or 8 internucleotidic linkages which are not chiral (as a non-limiting example, a phosphodiester). In some embodiments, a common pattern of backbone chiral centers comprises 10, 11, 12, 13, 14, 15 or more internucleotidic linkages in the Sp configuration, and no more than 8, no more than no more than 7, no more than 6, no more than 5, or no more than 4 internucleotidic linkages which are not chiral. In some embodiments, the internucleotidic linkages in the Sp configuration are optionally contiguous or not contiguous. In some embodiments, the internucleotidic linkages in the Rp configuration are optionally contiguous or not contiguous. In some embodiments, the internucleotidic linkages which are not chiral are optionally contiguous or not contiguous.

In some embodiments, the oligonucleotide comprises a block which is a stereochemistry block. For example, the oligonucleotide comprises a block which is an Rp block in that each internucleotidic linkage of the block is Rp. In some embodiments, the oligonucleotide comprises a block which is an Sp block in that each internucleotidic linkage of the block is Sp. In some embodiments, the oligonucleotide comprises a Rp block at the 5′-end. In some embodiments, the oligonucleotide comprises a Rp block at the 3′-end. In some embodiments, the oligonucleotide comprises a Sp block at the 5′-end. In some embodiments, the oligonucleotide comprises a Sp block at the 3′-end. In some embodiments, the oligonucleotide comprises both Rp and Sp blocks. In some embodiments, the oligonucleotide comprises one or more Rp but no Sp blocks. In some embodiments, the oligonucleotide comprises one or more Sp but no Rp blocks. In some embodiments, the oligonucleotide comprises one or more PO blocks wherein each internucleotidic linkage in a natural phosphate linkage.

In some embodiments, an adenosine in the oligonucleotide is followed by Sp. In some embodiments, an adenosine in the oligonucleotide is followed by Rp. In some embodiments, an adenosine in the oligonucleotie is followed by natural phosphate linkage (PO). In some embodiments, a uridine in the oligonucleotide is followed by Sp. In some embodiments, a uridine in the oligonucleotide is followed by Rp. In some embodiments, a uridine in the oligonucleotide is followed by natural phosphate linkage (PO). In some embodiments, a cytidine in the oligonucleotide is followed by Sp. In some embodiments, a cytidine in the oligonucleotide is followed by Rp. In some embodiments, a cytidine in the oligonucleotide is followed by natural phosphate linkage (PO). In some embodiments, a guanosine in the oligonucleotide is followed by Sp. In some embodiments, a guanosine in the oligonucleotide is followed by Rp. In some embodiments, a guanosine in the oligonucleotide is followed by natural phosphate linkage (PO). In some embodiments, cytidine and uridine are followed by Sp. In some embodiments, cytidine and uridine are followed by Rp. In some embodiments, cytidine and uridine are followed by natural phosphate linkage (PO). In some embodiments, adenosine and guanosine are followed by Sp. In some embodiments, adenosine and guanosine are followed by Rp.

Oligonucleotide modifications—sugar

In some embodiments of any one of the aspects described herein, the oligonucleotide further comprises, i.e., in addition to a nucleoside of Formula (II), a nucleoside with a modified sugar. By a “modified sugar” is meant a sugar or moiety other than 2′-deoxy (i.e, 2′-H) or 2′-OH ribose sugar. Some exemplary nucleotides comprising a modified sugar are 2′-F ribose, 2′-OMe ribose, 2′-0,4′-C-methylene ribose (locked nucleic acid, LNA), anhydrohexitol (1,5-anhydrohexitol nucleic acid, HNA), cyclohexene (Cyclohexene nucleic acid, CeNA), 2′-methoxyethyl ribose, 2′-O-allyl ribose, 2′-C-allyl ribose, 2′-O—N-methylacetamido (2′-O-NMA) ribose, a 2′-O-dimethylaminoethoxyethyl (2′-O-DMAEOE) ribose, 2′-O-aminopropyl (2′-O-AP) ribose, 2′-F arabinose (2′-ara-F), threose (Threose nucleic acid, TNA), and 2,3-dihydroxylpropyl (glycol nucleic acid, GNA). It is noted that the nucleoside with the modified sugar can be present at any position of the oligonucleotide.

In some embodiments, the oligonucleotide further comprises at least one, e.g., 1, 2, 3, 4, 5, 6, 7, 8, 9, 10 or more 2′-fluoro (2′-F) nucleotides. For example, the oligonucleotide can comprise 1, 2, 3, 4, 5, 6, 7, 8, 9 or 10 2′—F nucleotides. It is noted that the 2′-F nucleotides can be present at any position of the oligonucleotide.

In some embodiments, the oligonucleotide comprises, e.g., solely comprises nucleosides of Formula (II) and 2′-F nucleosides.

In some embodiments, the oligonucleotide further comprises at least one, e.g., 1, 2, 3, 4, 5, 6, 7, 8, 9, 10 or more 2′-OMe nucleotides. For example, the oligonucleotide can comprise 1, 2, 3, 4, 5, 6, 7, 8, 9 or 10 2′—OMe nucleotides. It is noted that the 2′-OMe nucleotides can be present at any position of the oligonucleotide.

In some embodiments, the oligonucleotide comprises, e.g., solely comprises solely comprises solely comprises nucleosides of Formula (II) and 2′-OMe nucleosides. In some other embodiments, the oligonucleotide comprises, e.g., solely comprises solely comprises nucleosides of Formula (II), 2′-OMe nucleosides and 2′-F nucleosides.

In some embodiments, the oligonucleotide further comprises at least one, e.g., 1, 2, 3, 4, 5, 6, 7, 8, 9, 10 or more 2′-deoxy, e.g., 2′-H nucleotides. For example, the oligonucleotide can comprise 1, 2, 3, 4, 5, 6, 7, 8, 9 or 10 of 2′-deoxy, e.g., 2′-H nucleotides. It is noted that the 2′-deoxy, e.g., 2′-H nucleotides can be present at any position of the oligonucleotide. For example, the oligonucleotide can comprise a 2′-deoxy, e.g., 2′-H nucleotide at 1, 2, 3, 4, 5 or 6 of positions 2, 5, 7, 12, 14 and 16, counting from 5′-end of the oligonucleotide. In some embodiments, the oligonucleotide comprises a 2′-deoxy nucleotide at positions 5 and 7, counting from 5′-end of the oligonucleotide.

In some embodiments, the oligonucleotide comprises, e.g., solely comprises solely comprises nucleosides of Formula (II) and 2′-deoxy (2′-H) nucleotides. In some embodiments, the oligonucleotide comprises, e.g., solely comprises nucleosides of Formula (I), 2′-OMe nucleosides, and 2′-deoxy (2′-H) nucleotides. In some embodiments, the oligonucleotide comprises, e.g., solely comprises nucleosides of Formula (I), 2′-F nucleosides and 2′-deoxy (2′-H) nucleotides. In some embodiments, the oligonucleotide comprises, e.g., solely comprises nucleosides of Formula (I), 2′-OMe nucleosides, 2′-F nucleosides and 2′-deoxy (2′-H) nucleotides.

### Oligonucleotides

It is noted that the nucleoside of Formula (II) can be located anywhere in the oligonucleotide. In some embodiments, the nucleoside of Formula (II) is present at the 5′- or 3′-terminus of the oligonucleotide. In some embodiments, the nucleoside of Formula (II) is present at an internal position of the oligonucleotide.

In some embodiments of any one of the aspects described herein, the oligonucleotide further comprises, i.e., in addition to a nucleotiside of Formula (II), a nucleoside with a modified sugar. By a “modified sugar” is meant a sugar or moiety other than 2′-deoxy (i.e, 2′-H) or 2′-OH ribose sugar. Some exemplary nucleotides comprising a modified sugar are 2′-F ribose, 2′-OMe ribose, 2′-O,4′-C-methylene ribose (locked nucleic acid, LNA), anhydrohexitol (1,5-anhydrohexitol nucleic acid, HNA), cyclohexene (Cyclohexene nucleic acid, CeNA), 2′-methoxyethyl ribose, 2′-O-allyl ribose, 2′-C-allyl ribose, 2′-O—N-methylacetamido (2′-O-NMA) ribose, a 2′-O-dimethylaminoethoxyethyl (2′-O-DMAEOE) ribose, 2′-O-aminopropyl (2′-O-AP) ribose, 2′-F arabinose (2′-ara-F), threose (Threose nucleic acid, TNA), and 2,3-dihydroxypropyl (glycol nucleic acid, GNA). It is noted that the nucleoside with the modified sugar can be present at any position of the oligonucleotide.

In some embodiments, the oligonucleotide further comprises at least one, e.g., 1, 2, 3, 4, 5, 6, 7, 8, 9, 10 or more 2′-fluoro (2′-F) nucleotides. For example, the oligonucleotide can comprise 1, 2, 3, 4, 5, 6, 7, 8, 9 or 10 2′—F nucleotides. It is noted that the 2′-F nucleotides can be present at any position of the oligonucleotide.

In some embodiments, the oligonucleotide comprises, e.g., solely comprises 2′-nucleosides of Formula (II) and 2′-F nucleosides.

In some embodiments, the oligonucleotide further comprises at least one, e.g., 1, 2, 3, 4, 5, 6, 7, 8, 9, 10 or more 2′-OMe nucleotides. For example, the oligonucleotide can comprise 1, 2, 3, 4, 5, 6, 7, 8, 9 or 10 2′—OMe nucleotides. It is noted that the 2′-OMe nucleotides can be present at any position of the oligonucleotide.

In some embodiments, the oligonucleotide comprises, e.g., solely comprises solely comprises solely comprises 2′-nucleosides of Formula (II) and 2′-OMe nucleosides. In some other embodiments, the oligonucleotide comprises, e.g., solely comprises solely comprises 2′-nucleosides of Formula (II), 2′-OMe nucleosides and 2′-F nucleosides.

In some embodiments, the oligonucleotide further comprises at least one, e.g., 1, 2, 3, 4, 5, 6, 7, 8, 9, 10 or more 2′-deoxy, e.g., 2′-H nucleotides. For example, the oligonucleotide can comprise 1, 2, 3, 4, 5, 6, 7, 8, 9 or 10 of 2′-deoxy, e.g., 2′-H nucleotides. It is noted that the 2′-deoxy, e.g., 2′-H nucleotides can be present at any position of the oligonucleotide. For example, the oligonucleotide can comprise a 2′-deoxy, e.g., 2′-H nucleotide at 1, 2, 3, 4, 5 or 6 of positions 2, 5, 7, 12, 14 and 16, counting from 5′-end of the oligonucleotide. In some embodiments, the oligonucleotide comprises a 2′-deoxy nucleotide at positions 5 and 7, counting from 5′-end of the oligonucleotide.

In some embodiments, the oligonucleotide comprises, e.g., solely comprises solely comprises nucleosides of Formula (II) and 2′-deoxy (2′-H) nucleotides. In some embodiments, the oligonucleotide comprises, e.g., solely comprises nucleosides of Formula (II), 2′-OMe nucleosides, and 2′-deoxy (2′-H) nucleotides. In some embodiments, the oligonucleotide comprises, e.g., solely comprises nucleosides of Formula (II), 2′-F nucleosides and 2′-deoxy (2′-H) nucleotides. In some embodiments, the oligonucleotide comprises, e.g., solely comprises nucleosides of Formula (II), 2′-OMe nucleosides, 2′-F nucleosides and 2′-deoxy (2′-H) nucleotides.

In some embodiments of any one of the aspects described herein, the oligonucleotide further comprises, i.e., in addition to a nucleoside of Formula (II), a non-natural nucleobase. In some embodiments, the oligonucleotide can comprise one or more, e.g., 1, 2, 3, 4, 5, 6, 7, 8, 9, 10 or more nucleotides comprising an independently selected non-natural nucleobase. When present, a nucleotide comprising a non-natural nucleobase can be present anywhere in the oligonucleotide.

In some embodiments, the oligonucleotide further comprises a solid support linked thereto.

The oligonucleotides described herein can range from few nucleotides (e.g., 2, 3, 4, 5, 6, 7, 8, 9 or 10 nucleotides) in length to hundreds of nucleotides in length. For example, the oligonucleotide can be from 5 nucleotides to 100 nucleotides in length. In some embodiments, the oligonucleotide is from 10 nucleotides to 50 nucleotides in length. For example, the oligonucleotide is between 15 and 35, more generally between 18 and 25, yet more generally between 19 and 24, and most generally between 19 and 21 base pairs in length. In some embodiments, longer oligonucleotides of between 25 and 30 nucleotides in length are preferred. In some embodiments, shorter oligonucleotides of between 10 and 15 nucleotides in length are preferred. In another embodiment, the oligonucleotide is at least 21 nucleotides in length.

5′-modifications

In some embodiments of any one of the aspects described herein, the oligonucleotides described herein are 5′ phosphorylated or include a phosphoryl analog at the 5′ prime terminus. 5′-phosphate modifications include those which are compatible with RISC mediated gene silencing. Suitable modifications include: 5′-monophosphate ((HO)₂(O)P—O-5′); 5′-diphosphate ((HO)₂(O)P—O—P(HO)(O)—O-5′); 5′-triphosphate ((HO)₂(O)P—O—(HO)(O)P—O—P(HO)(O)—O-5′); 5′-guanosine cap (7-methylated or non-methylated) (7m-G-O-5′-(HO)(O)P—O—(HO)(O)P—O—P(HO)(O)—O-5′); 5′-adenosine cap (Appp), and any modified or unmodified nucleotide cap structure (N—O-5′-(HO)(O)P—O—(HO)(O)P—O—P(HO)(O)—O-5′); 5′-monothiophosphate (phosphorothioate; (HO)₂(S)P—O-5′); 5′-monodithiophosphate (phosphorodithioate; (HO)(HS)(S)P—O-5′), 5′-phosphorothiolate ((HO)₂(O)P—S-5′); any additional combination of oxygen/sulfur replaced monophosphate, diphosphate and triphosphates (e.g. 5′-alpha-thiotriphosphate, 5′-gamma-thiotriphosphate, etc.), 5′-phosphoramidates ((HO)₂(O)P—NH-5′, (HO)(NH₂)(O)P—O-5′), 5′-alkylphosphonates (e.g., RP(OH)(O)—O-5′-, R=alkyl, e.g., methyl, ethyl, isopropyl, propyl, etc.), 5′-alkenylphosphonates (i.e. vinyl, substituted vinyl, e.g., OH)₂(O)P-5′-CH═ or (OH)₂(O)P-5′-CH₂—), 5′-alkyletherphosphonates (e.g., R(OH)(O)P—O-5′, R=alkylether, e.g., methoxymethyl (MeOCH2-), ethoxymethyl, etc.) Other exemplary 5′-modifications include where Z is optionally substituted alkyl at least once, e.g., ((HO)₂(X)P—O[—(CH₂)ₐ—O—P(X)(OH)—O]_{b}-5′, ((HO)₂(X)P—O[—(CH₂)ₐ—P(X)(OH)—O]_{b}-5′, ((HO)₂(X)P—[—(CH₂)ₐ—O—P(X)(OH)—O]_{b}-5′; dialkyl terminal phosphates and phosphate mimics: HO[—(CH₂)ₐ—O—P(X)(OH)—O]_{b}-5′, H₂N[—(CH₂)ₐ—O—P(X)(OH)—O]_{b}-5′, H[—(CH₂)ₐ—O—P(X)(OH)—O]_{b}-5′, Me₂N[—(CH₂)ₐ—O—P(X)(OH)—O]_{b}-5′, HO[—(CH₂)ₐ—P(X)(OH)—O]_{b}-5′, H₂N[—(CH₂)ₐ—P(X)(OH)—O]_{b}-5′, H[—(CH₂)ₐ—P(X)(OH)—O]_{b}-5′, Me₂N[—(CH₂)ₐ—P(X)(OH)—O]_{b}-5′, wherein a and b are each independently 1-10. Other embodiments, include replacement of oxygen and/or sulfur with BH₃, BH₃⁻ and/or Se.

In some embodiments of any one of the aspects described herein, the oligonucleotide comprises a 5′-vinylphosphonate group. For example, the oligonucleotide comprises a 5′-E-vinyl phosphonate group. In some other non-limiting example, the oligonucleotide comprises a 5′-Z-vinylphosphonate group.

In some embodiments of any one of the aspects, the oligonucleotide described herein comprises a 5′-morpholino, a 5′-dimethylamino, a 5′-deoxy, an inverted abasic, or an inverted abasic locked nucleic acid modification at the 5′-end.

In some embodiments of any one of the aspects, the oligonucleotide described herein can comprise a thermally destabilizing modification. For example, the oligonucleotide can comprise at least one thermally destabilizing modification of the duplex within the first 9 nucleotide positions, counting from the 5′-end of the oligonucleotide. In some embodiments, the thermally destabilizing modification is located at position 2, 3, 4, 5, 6, 7, 8 or 9, counting from the 5′-end of the antisense strand. In some embodiments, thermally destabilizing modification is located in positions 2-9, or preferably positions 4-8, counting from the 5′-end of the oligonucleotide. In some further embodiments, the thermally destabilizing modification is located at position 5, 6, 7 or 8, counting from the 5′-end of the oligonucleotide. In still some further embodiments, the thermally destabilizing modification is located at position 7, counting from the 5′-end of the oligonucleotide.

The term “thermally destabilizing modification(s)” includes modification(s) that would result with a dsRNA with a lower overall melting temperature (Tm) (preferably a Tm with one, two, three or four degrees lower than the Tm of the dsRNA without having such modification(s). In some embodiments, the thermally destabilizing modification is located at position 2, 3, 4, 5, 6, 7, 8 or 9, counting from the 5′-end of the antisense strand.

The thermally destabilizing modifications can include, but are not limited to, abasic modification; mismatch with the opposing nucleotide in the opposing strand; and sugar modification such as 2′-deoxy modification or acyclic nucleotide, e.g., unlocked nucleic acids (UNA) or glycol nucleic acid (GNA). For example, the thermally destabilizing modifications can include, but are not limited to, mUNA and GNA building blocks as follows:

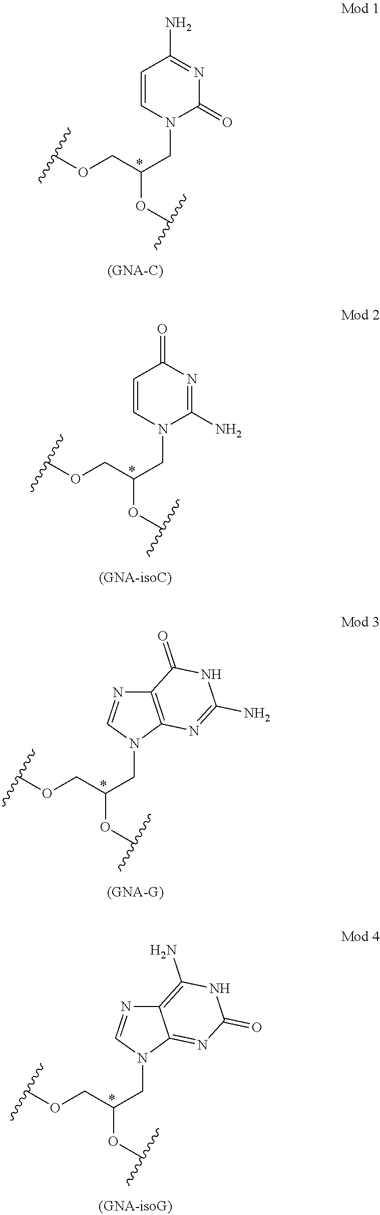 [SMILES: *OCC(Cn1ccc(N)nc1=O)O* ; *OCC(Cn1ccc(=O)nc1N)O* ; *OCC(Cn1cnc2c(=O)[nH]c(N)nc21)O* ; *OCC(Cn1cnc2c(N)[nH]c(=O)nc21)O*]

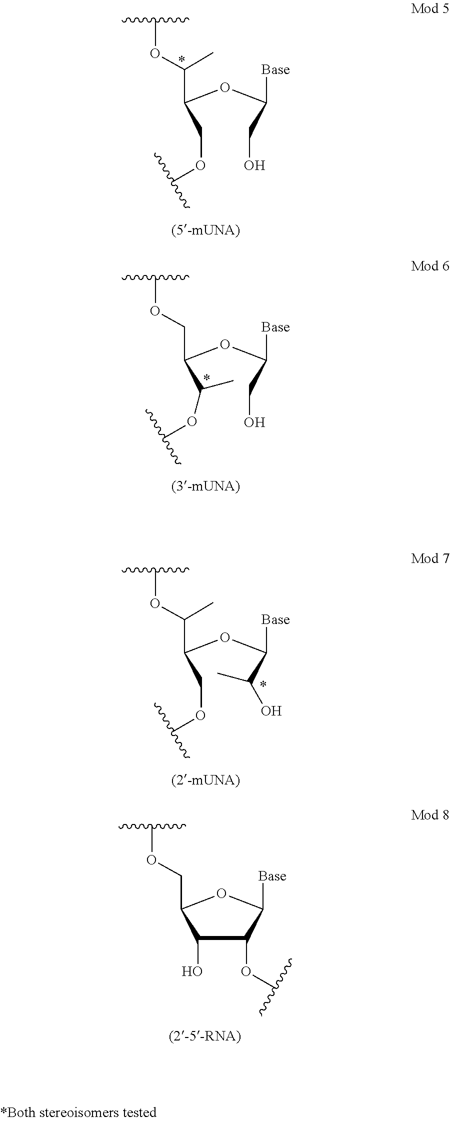

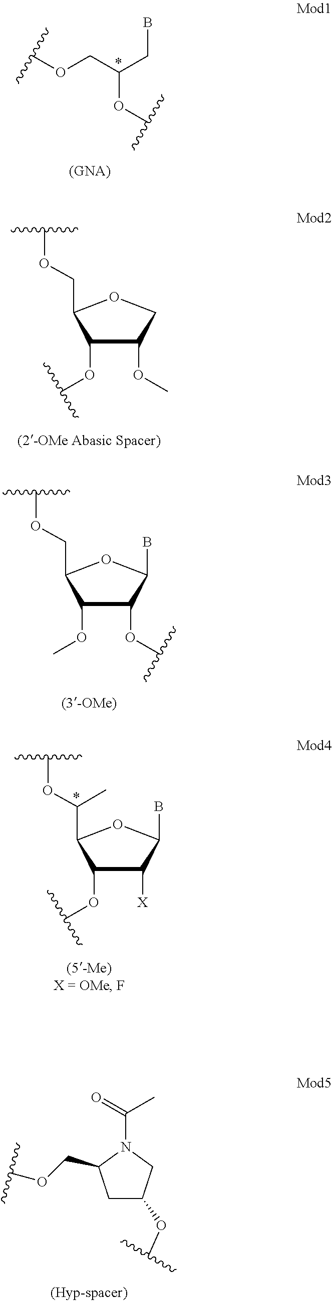

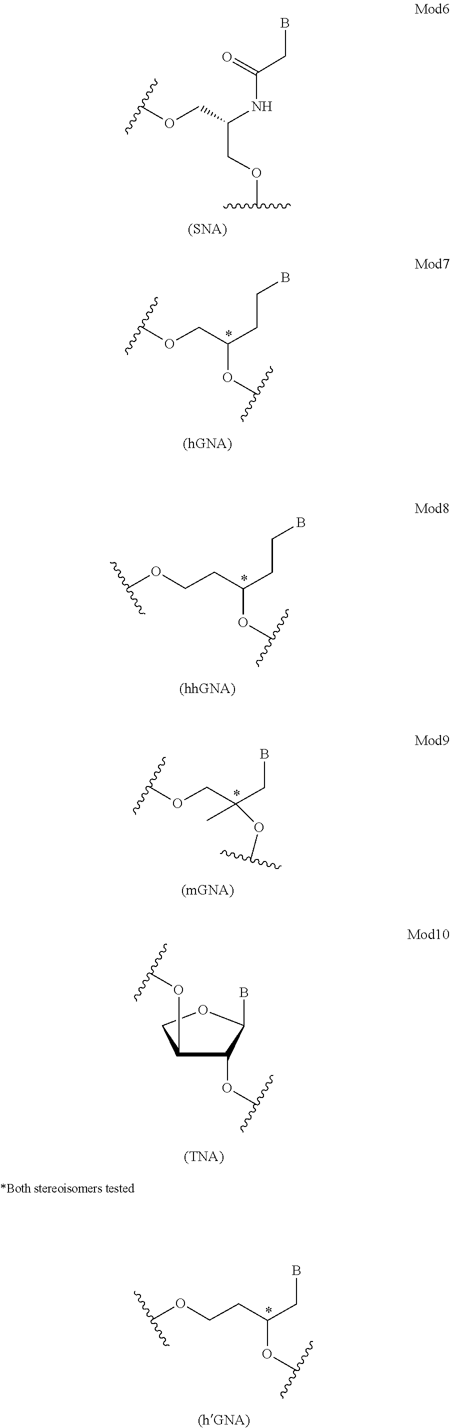

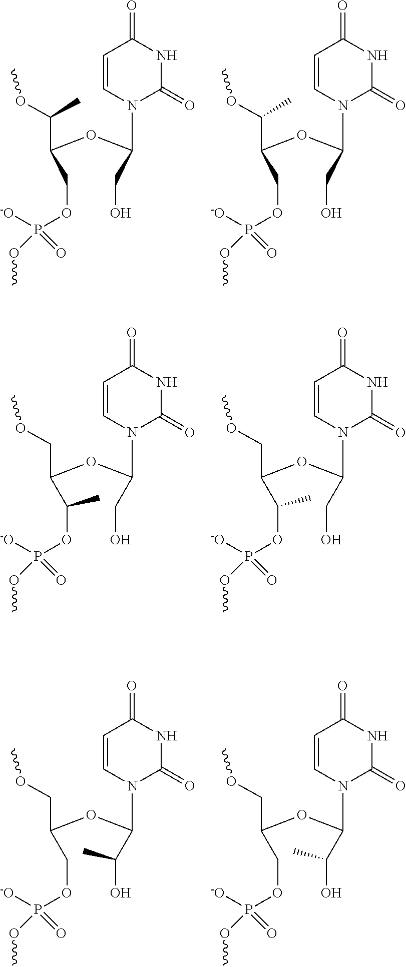

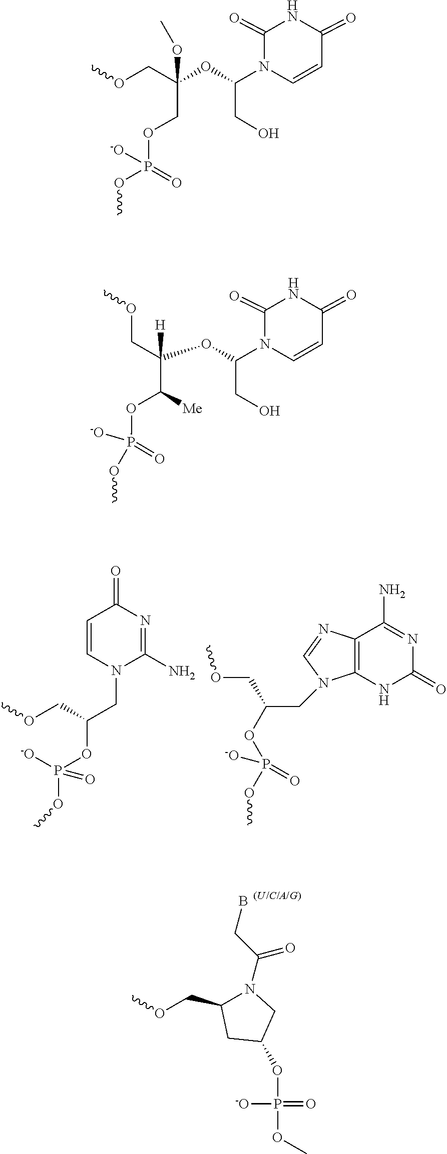

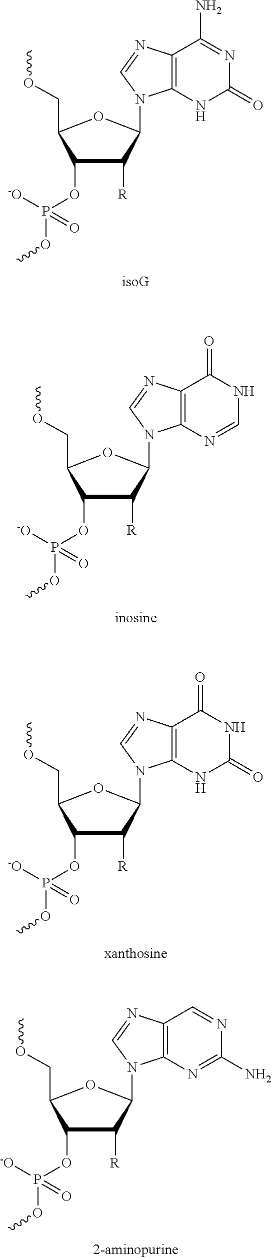

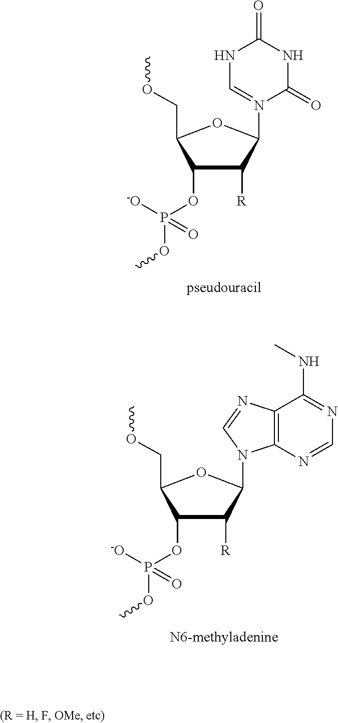

In some embodiments, the destabilizing modification is selected from the group consisting of GNA-isoC, GNA-isoG, 5′-mUNA, 4′-mUNA, 3′-mUNA, and 2′-mUNA.

In some embodiments, the destabilizing modification mUNA is selected from the group consisting of

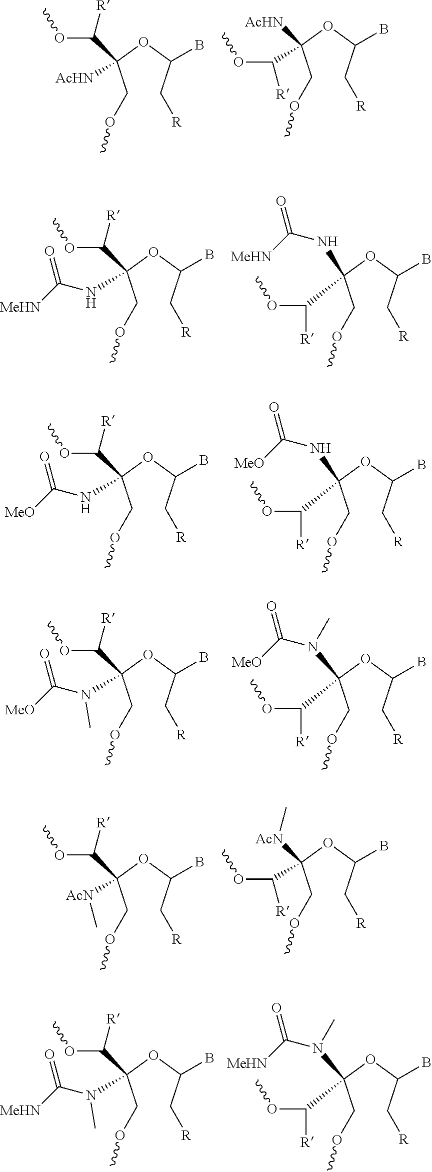

R=H, OH; OMe; Cl, F; OH; O—(CH₂)₂OMe; SMe, NMe₂; NH₂; Me; CCH (alkyne), O-nPr; O-alkyl; O-alkylamino;
R′=H, Me;
B=A; C; 5-Me-C; G; I; U; T; Y; 2-thiouridine; 4-thiouridine; C5-modified pyrimidines; C2-modified purines; N8-modified purines; phenoxazine; G-clamp; non-canonical mono, bi and tricyclic heterocycles; pseudouracil; isoC; isoG; 2,6-diamninopurine; pseudocytosine; 2-aminopurine; xanthosine; N6-alkyl-A; O6-alkyl-G; 2-thiouridine; 4-thiouridine; C5-modified pyrimidines; C2-modified purines; N8-modified purines; 7-deazapurines, phenoxazine; G-clamp; non-canonical mono, bi and tricyclic heterocycles; and
Stereochemistry is R or S and combination of R and S for the unspecified chiral centers.

In some embodiments, the destabilizing modification mUNA is selected from the group consisting of

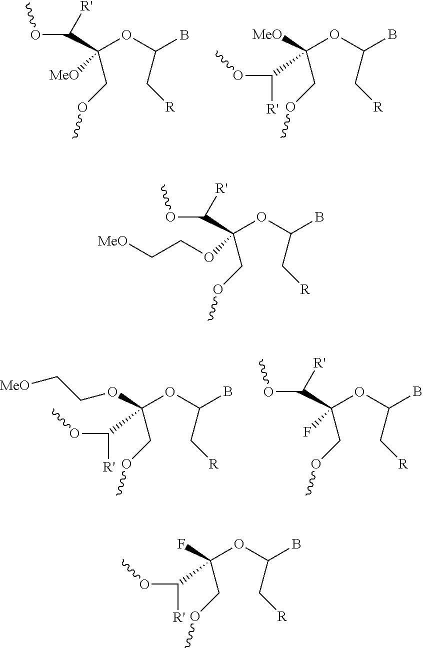

R=H, OH; OMe; Cl, F; OH; O—(CH₂)₂OMe; SMe, NMe₂; NH₂; Me; CCH (alkyne), O-nPr; O-alkyl; O-alkylamino;
R′=H, Me;
B=A; C; 5-Me-C; G; I; U; T; Y; 2-thiouridine; 4-thiouridine; C5-modified pyrimidines; C2-modified purines; N8-modified purines; phenoxazine; G-clamp; non-canonical mono, bi and tricyclic heterocycles; pseudouracil; isoC; isoG; 2,6-diamninopurine; pseudocytosine; 2-aminopurine; xanthosine; N6-alkyl-A; O6-alkyl-G; 2-thiouridine; 4-thiouridine; C5-modified pyrimidines; C2-modified purines; N8-modified purines; 7-deazapurines, phenoxazine; G-clamp; non-canonical mono, bi and tricyclic heterocycles; and
Stereochemistry is R or S and combination of R and S for the unspecified chiral centers.

In some embodiments, the destabilizing modification mUNA is selected from the group consisting of

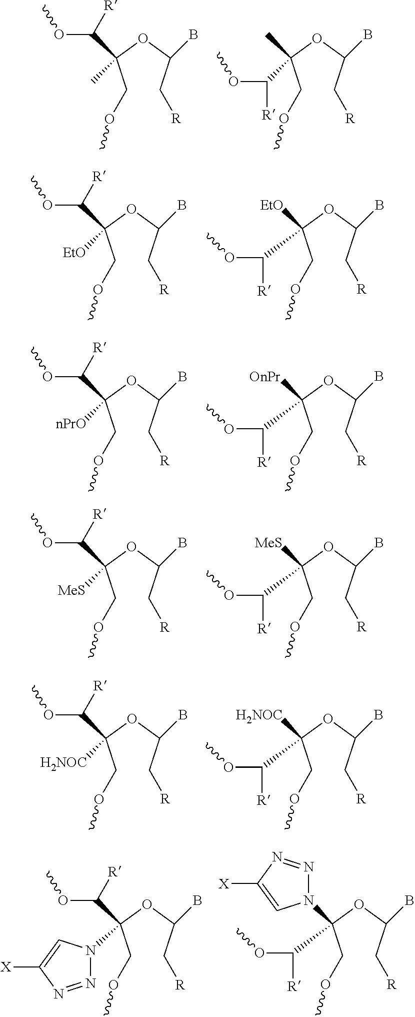

R=H, OMe; F; OH; O—(CH₂)₂OMe; SMe, NMe₂; NH₂; Me; O-nPr; O-alkyl; O-alkylamino;
R′=H, Me;
B=A; C; 5-Me-C; G; I; U; T; Y; 2-thiouridine; 4-thiouridine; C5-modified pyrimidines; C2-modified purines; N8-modified purines; phenoxazine; G-clamp; non-canonical mono, bi and tricyclic heterocycles; pseudouracil; isoC; isoG; 2,6-diamninopurine; pseudocytosine; 2-aminopurine; xanthosine; N6-alkyl-A; O6-alkyl-G; 7-deazapurines; and
Stereochemistry is R or S and combination of R and S for the unspecified chiral centers.

In some embodiments, the destabilizing modification mUNA is selected from the group consisting of

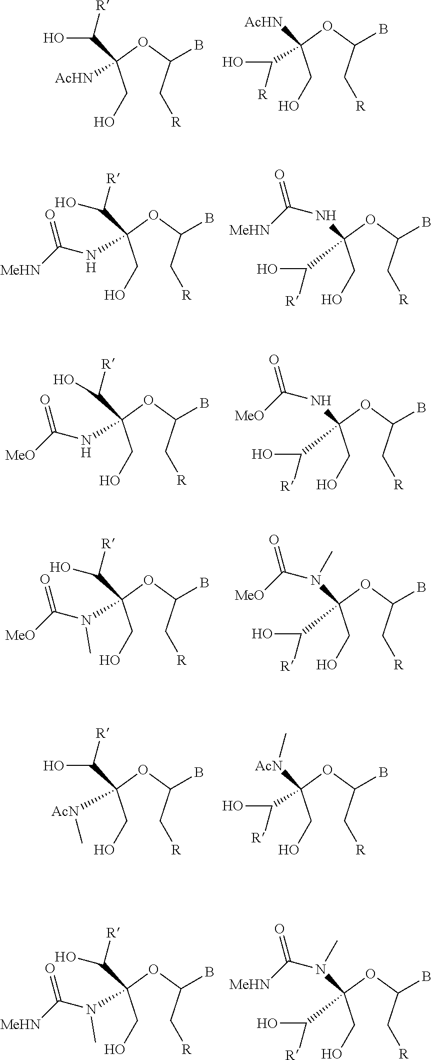

R=H, OH; OMe; Cl, F; OH; O—(CH₂)₂OMe; SMe, NMe₂; NH₂; Me; CCH (alkyne), O-nPr; O-alkyl; O-alkylamino;
R′=H, Me;
B=A; C; 5-Me-C; G; I; U; T; Y; 2-thiouridine; 4-thiouridine; C5-modified pyrimidines; C2-modified purines; N8-modified purines; phenoxazine; G-clamp; non-canonical mono, bi and tricyclic heterocycles; pseudouracil; isoC; isoG; 2,6-diamninopurine; pseudocytosine; 2-aminopurine; xanthosine; N6-alkyl-A; O6-alkyl-G; 2-thiouridine; 4-thiouridine; C5-modified pyrimidines; C2-modified purines; N8-modified purines; 7-deazapurines, phenoxazine; G-clamp; non-canonical mono, bi and tricyclic heterocycles; and
Stereochemistry is R or S and combination of R and S for the unspecified chiral centers

In some embodiments, the destabilizing modification mUNA is selected from the group consisting of

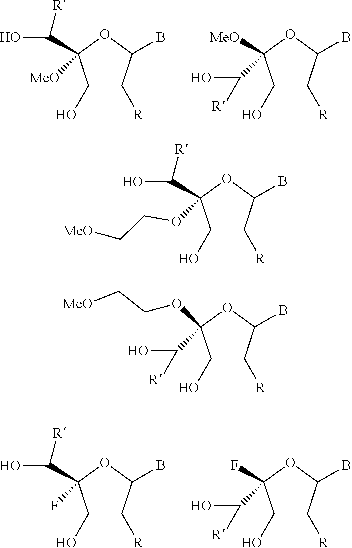

R=H, OH; OMe; Cl, F; OH; O—(CH₂)₂OMe; SMe, NMe₂; NH₂; Me; CCH (alkyne), O-nPr; O-alkyl; O-alkylamino;
R′=H, Me;
B=A; C; 5-Me-C; G; I; U; T; Y; 2-thiouridine; 4-thiouridine; C5-modified pyrimidines; C2-modified purines; N8-modified purines; phenoxazine; G-clamp; non-canonical mono, bi and tricyclic heterocycles; pseudouracil; isoC; isoG; 2,6-diamninopurine; pseudocytosine; 2-aminopurine; xanthosine; N6-alkyl-A; O6-alkyl-G; 2-thiouridine; 4-thiouridine; C5-modified pyrimidines; C2-modified purines; N8-modified purines; 7-deazapurines, phenoxazine; G-clamp; non-canonical mono, bi and tricyclic heterocycles; and
Stereochemistry is R or S and combination of R and S for the unspecified chiral centers

In some embodiments, the modification mUNA is selected from the group consisting of

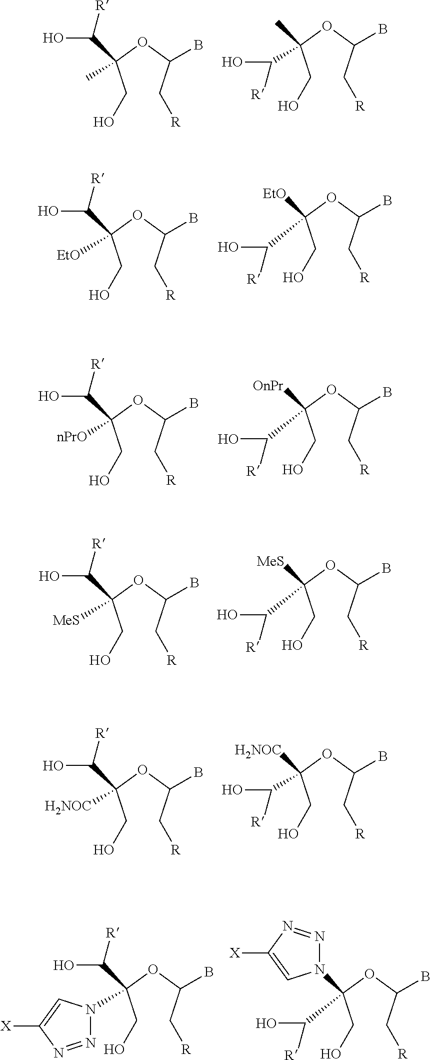

R=H, OMe; F; OH; O—(CH₂)₂OMe; SMe, NMe₂; NH₂; Me; O-nPr; O-alkyl; O-alkylamino;
R′=H, Me;
B=A; C; 5-Me-C; G; I; U; T; Y; 2-thiouridine; 4-thiouridine; C5-modified pyrimidines; C2-modified purines; N8-modified purines; phenoxazine; G-clamp; non-canonical mono, bi and tricyclic heterocycles; pseudouracil; isoC; isoG; 2,6-diamninopurine; pseudocytosine; 2-aminopurine; xanthosine; N6-alkyl-A; O6-alkyl-G; 7-deazapurines; and
Stereochemistry is R or S and combination of R and S for the unspecified chiral centers

Exemplary abasic modifications include, but are not limited to the following:

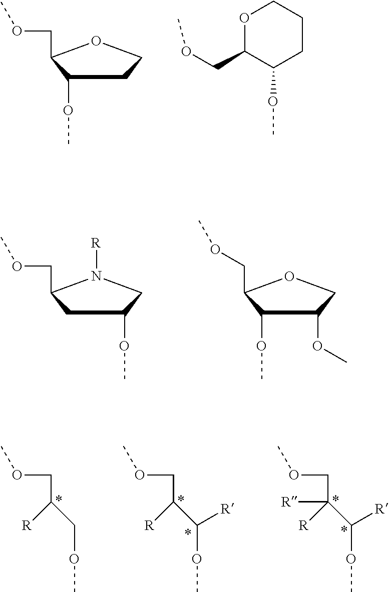

Wherein R=H, Me, Et or OMe; R′=H, Me, Et or OMe; R″=H, Me, Et or OMe

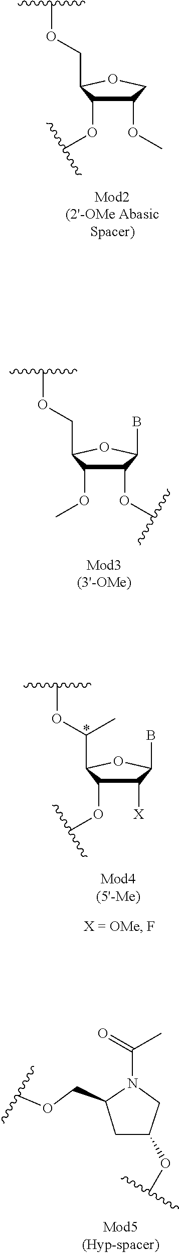

wherein B is a modified or unmodified nucleobase and the asterisk on each structure represents either R, S or racemic.

Exemplified sugar modifications include, but are not limited to the following:

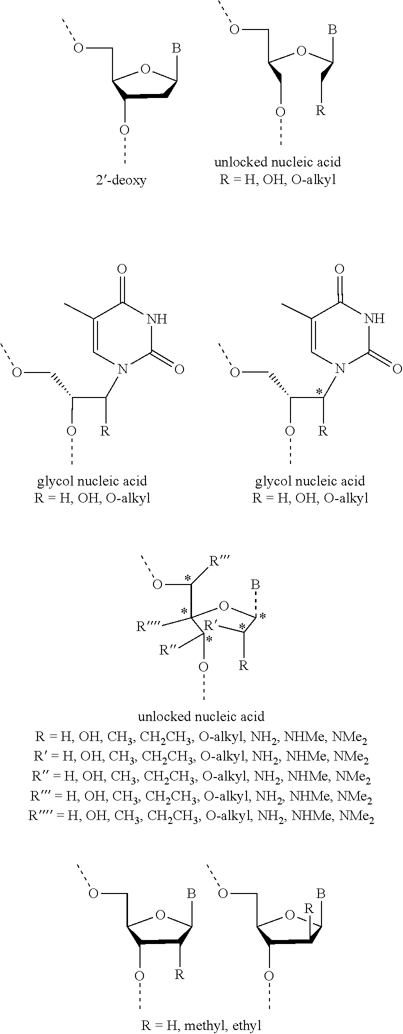

wherein B is a modified or unmodified nucleobase and the asterisk on each structure represents either R, S or racemic.

In some embodiments the thermally destabilizing modification of the duplex is selected from the mUNA and GNA building blocks described in Examples 1-3 herein. In some embodiments, the destabilizing modification is selected from the group consisting of GNA-isoC, GNA-isoG, 5′-mUNA, 4′-mUNA, 3′-mUNA, and 2′-mUNA. In some further embodiments of this, the dsRNA molecule further comprises at least one thermally destabilizing modification selected from the group consisting of GNA, 2′-OMe, 3′-OMe, 5′-Me, Hy p-spacer, SNA, hGNA, hhGNA, mGNA, TNA and h'GNA (Mod A-Mod K).

The term “acyclic nucleotide” refers to any nucleotide having an acyclic ribose sugar, for example, where any of bonds between the ribose carbons (e.g., C1′—C2′, C2′—C3′, C3′—C4′, C4′—O4′, or C1′—O4′) is absent and/or at least one of ribose carbons or oxygen (e.g., C1′, C2′, C3′, C4′ or O4′) are independently or in combination absent from the nucleotide. In some embodiments, acyclic nucleotide is

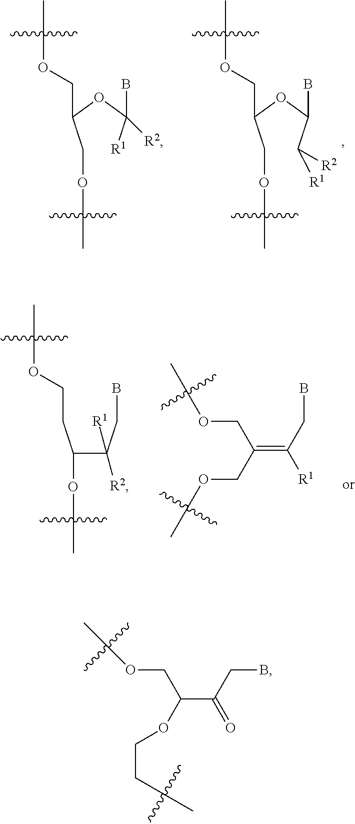

wherein B is a modified or unmodified nucleobase, R1 and R2 independently are H, halogen, OR3, or alkyl; and R3 is H, alkyl, cycloalkyl, aryl, aralkyl, heteroaryl or sugar). The term “UNA” refers to unlocked acyclic nucleic acid, wherein any of the bonds of the sugar has been removed, forming an unlocked “sugar” residue. In one example, UNA also encompasses monomers with bonds between C1′—C4′ being removed (i.e. the covalent carbon-oxygen-carbon bond between the C1′ and C4′ carbons). In another example, the C2′—C3′ bond (i.e. the covalent carbon-carbon bond between the C2′ and C3′ carbons) of the sugar is removed (see Mikhailov et. al., Tetrahedron Letters, 26 (17): 2059 (1985); and Fluiter et al., Mol. Biosyst., 10: 1039 (2009), which are hereby incorporated by reference in their entirety). The acyclic derivative provides greater backbone flexibility without affecting the Watson-Crick pairings. The acyclic nucleotide can be linked via 2′-5′ or 3′-5′ linkage.

The term ‘GNA’ refers to glycol nucleic acid which is a polymer similar to DNA or RNA but differing in the composition of its “backbone” in that is composed of repeating glycerol units linked by phosphodiester bonds:

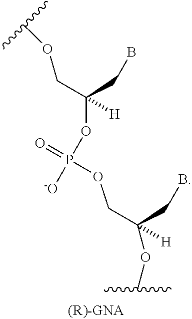

The thermally destabilizing modification of the duplex can be mismatches (i.e., noncomplementary base pairs) between the thermally destabilizing nucleotide and the opposing nucleotide in the opposite strand within the dsRNA duplex. Exemplary mismatch base pairs include G:G, G:A, G:U, G:T, A:A, A:C, C:C, C:U, C:T, U:U, T:T, U:T, or a combination thereof. Other mismatch base pairings known in the art are also amenable to the present invention. A mismatch can occur between nucleotides that are either naturally occurring nucleotides or modified nucleotides, i.e., the mismatch base pairing can occur between the nucleobases from respective nucleotides independent of the modifications on the ribose sugars of the nucleotides. In certain embodiments, the dsRNA molecule contains at least one nucleobase in the mismatch pairing that is a 2′-deoxy nucleobase; e.g., the 2′-deoxy nucleobase is in the sense strand.

In some embodiments, the thermally destabilizing modification of the duplex in the seed region of the antisense strand includes nucleotides with impaired W—C H-bonding to complementary base on the target mRNA, such as:

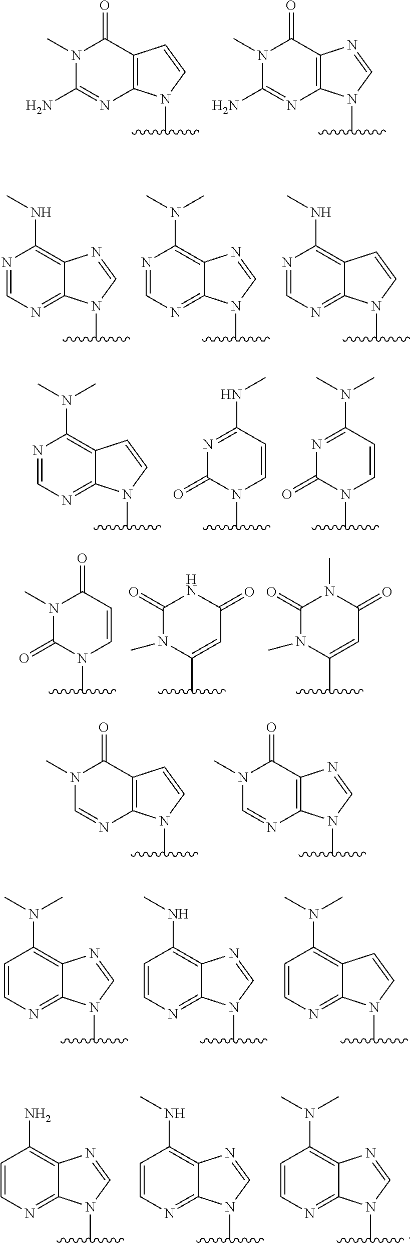

More examples of abasic nucleotide, acyclic nucleotide modifications (including UNA and GINA), and mismatch modifications have been described in detail in WO 2011/133876, which is herein incorporated by reference in its entirety.

The thermally destabilizing modifications may also include universal base with reduced or abolished capability to form hydrogen bonds with the opposing bases, and phosphate modifications.

In some embodiments, the thermally destabilizing modification includes nucleotides with non-canonical bases such as, but not limited to, nucleobase modifications with impaired or completely abolished capability to form hydrogen bonds with bases in the opposite strand. These nucleobase modifications have been evaluated for destabilization of the central region of the dsRNA duplex as described in WO 2010/0011895, which is herein incorporated by reference in its entirety. Exemplary nucleobase modifications are:

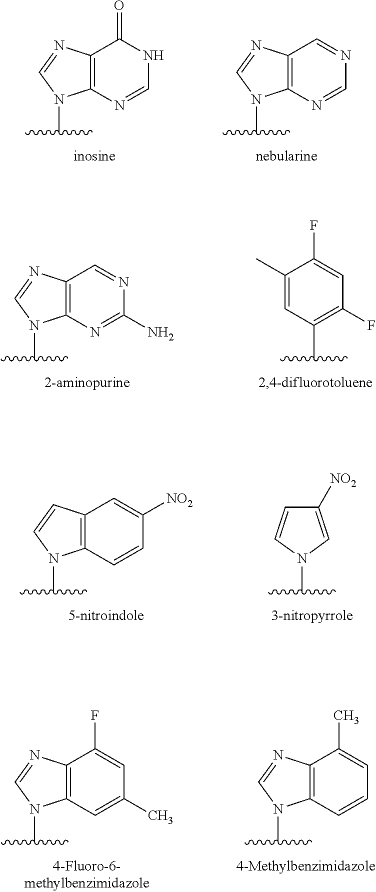

In some embodiments, the thermally destabilizing modification of the duplex in the seed region of the antisense strand includes one or more a-nucleotide complementary to the base on the target mRNA, such as:

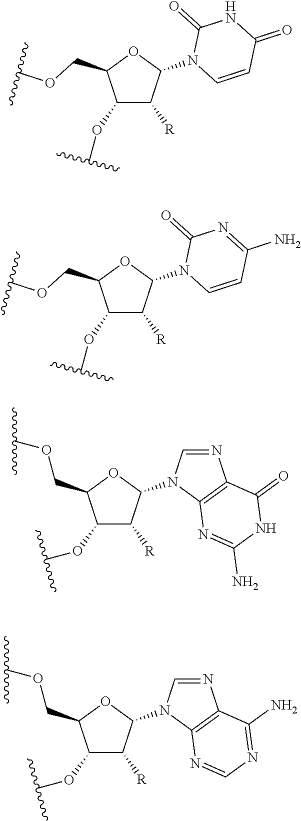

wherein R is H, OH, OCH₃, F, NH₂, NHMe, NMe₂ or O-alkyl

Exemplary phosphate modifications known to decrease the thermal stability of dsRNA duplexes compared to natural phosphodiester linkages are:

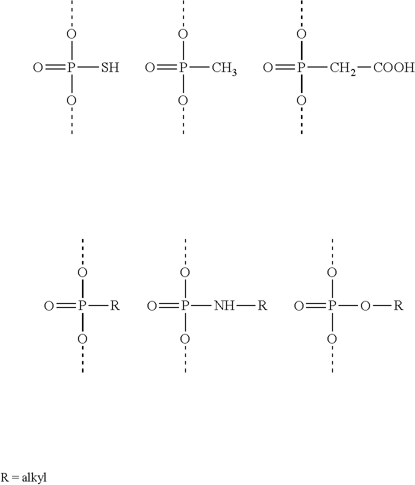

The alkyl for the R group can be a C₁-C₆alkyl. Specific alkyls for the R group include, but are not limited to methyl, ethyl, propyl, isopropyl, butyl, pentyl and hexyl.

In some embodiments of any one of the aspects described herein, the oligonucleotide can comprise one or more stabilizing modifications. For example, the oligonucleotide can comprise at least two (e.g., two, three, four, five, six, seven, eight, nine, ten or more) stabilizing modifications.

In some embodiments, the oligonucleotide comprises at least two (e.g., two, three, four, five, six, seven, eight, nine, ten or more) stabilizing modifications. Without limitations, a stabilizing modification in the oligonucleotide can be present at any positions. In some embodiments, the oligonucleotide comprises stabilizing modifications at positions 2, 6, 8, 9, 14 and 16, counting from the 5′-end. In some other embodiments, the oligonucleotide comprises stabilizing modifications at positions 2, 6, 14 and 16, counting from the 5′-end. In still some other embodiments, the oligonucleotide comprises stabilizing modifications at positions 2, 14 and 16, counting from the 5′-end. In some embodiments, the oligonucleotide comprises stabilizing modifications at positions 7, 10 and 11, counting from the 5′-end. In some other embodiments, the oligonucleotide comprises stabilizing modifications at positions 7, 9, 10 and 11, counting from the 5′-end.

In some embodiments, the oligonucleotide comprises at least one stabilizing modification adjacent to a destabilizing modification. For example, the stabilizing modification can be the nucleotide at the 5′-end or the 3′-end of the destabilizing modification, i.e., at position −1 or +1 from the position of the destabilizing modification. In some embodiments, the oligonucleotide comprises a stabilizing modification at each of the 5′-end and the 3′-end of the destabilizing modification, i.e., positions −1 and +1 from the position of the destabilizing modification.

In some embodiments, the oligonucleotide comprises at least two stabilizing modifications at the 3′-end of a destabilizing modification, i.e., at positions +1 and +2 from the position of the destabilizing modification.

Exemplary thermally stabilizing modifications include, but are not limited to 2′-fluoro modifications. Other thermally stabilizing modifications include, but are not limited to LNA.

### Double-Stranded RNAs

The skilled person is well aware that double-stranded RNAs comprising a duplex structure of between 20 and 23, but specifically 21, base pairs have been hailed as particularly effective in inducing RNA interference (Elbashir et al., EMBO 2001, 20:6877-6888). However, others have found that shorter or longer double-stranded oligonucleotides can be effective as well.

Accordingly, in one aspect, provided herein is a double-stranded RNA (dsRNA) comprising a first strand (also referred to as an antisense strand or a guide strand) and a second strand (also referred to as a sense strand or passenger strand, wherein at least one of the first (i.e., the antisense strand) or the second strand (i.e., the sense strand) is an oligonucleotide described herein. In other words, at least one of the first (i.e., the antisense strand) or the second strand (i.e., the sense strand) comprises at least one nucleotide of Formula (II).

In some embodiments of the any one of the aspects described herein, the antisense strand is substantially complementary to a target nucleic acid, e.g., a target gene or mRNA gene and the dsRNA is capable of inducing targeted cleavage of the target nucleic acid. Without limitations, the dsRNAs of the invention can be substituted for the dsRNA molecules and can be used in RNA interference based gene silencing techniques, including, but not limited to, in vitro or in vivo applications.

In some embodiments of any one of the aspects described herein, the sense strand is an oligonucleotide described herein. In other words, the sense strand comprises at least one nucleotide of Formula (II).

In some embodiments of any one of the aspects described herein, the antisense strand is an oligonucleotide described herein. In other words, the antisense strand comprises at least one nucleotide of Formula (II).

As described herein, the dsRNA molecule described herein can comprise at least one, e.g., 1, 2, 3, 4, 5, 6, 7, 8, 9, 10 or more of nucleotide of Formula (II). Without limitations, the nucleotides of Formula (II) all can be present in one strand. The nucleotide of Formula (II) may occur on any nucleotide of the sense strand or antisense strand or both in any position of the strand.

In some embodiments, the sense strand comprises 1, 2, 3, 4, 5, 6, 7, 8, 9, 10 or more nucleotides of Formula (II) described herein. The nucleotide of Formula (II) described herein can be present at any position of the sense strand. For example, the nucleotide of Formula (II) described herein can be present at a terminal region of the sense strand. For example, the nucleotide of Formula (II) described herein can be present at one or more of positions 1, 2, 3 and 4, counting from the 5′-end of the sense strand. In another non-limiting example, the nucleotide of Formula (II) described herein can be present at one or more of positions 1, 2, 3 and 4, counting from the 3′-end of the sense strand. In some embodiments, the nucleotide of Formula (II) can be present at one or more of positions 18, 19, 20 and 21, counting from 5′-end of the sense strand. The nucleotide of Formula (II) described herein can also be located at a central region of sense strand. For example, the nucleotide of Formula (II) described herein can be located at one or more of positions 6, 7, 8, 9, 10, 11, 12 and 13, counting from 5′-end of the sense strand. In some embodiments, the nucleotide of Formula (II) is at the 5-terminus of the sense strand.

In some embodiments, the antisense strand comprises 1, 2, 3, 4, 5, 6, 7, 8, 9, 10 or more of nucleotides of Formula (II) described herein. The nucleotide of Formula (II) described herein can be present at any position of the antisense strand. For example, the nucleotide of Formula (II) described herein can be present at a terminal region of the antisense strand. For example, the nucleotide of Formula (II) described herein can be present at one or more of positions 1, 2, 3 and 4, counting from the 5′-end of the antisense strand. In another non-limiting example, the nucleotide of Formula (II) described herein nucleotide can be present at one or more of positions 1, 2, 3, 4, 5 and 6, counting from the 3′-end of the antisense strand. In some embodiments, the nucleotide of Formula (II) described herein nucleotide can be present at one or more of positions 18, 19, 20, 21, 22 and 23, counting from 5′-end of the antisense strand. The nucleotide of Formula (II) described herein nucleotide can also be located at a central region of the antisense strand. For example, the nucleotide of Formula (II) described herein nucleotide can be located at one or more of positions 6, 7, 8, 9, 10, 11, 12 and 13, counting from 5′-end of the antisense strand. In some embodiments, the nucleotide of Formula (II) is at the 3′-terminus of the antisense strand.

Each strand of the dsRNA molecule can range from 15-35 nucleotides in length. For example, each strand can be between, 17-35 nucleotides in length, 17-30 nucleotides in length, 25-35 nucleotides in length, 27-30 nucleotides in length, 17-23 nucleotides in length, 17-21 nucleotides in length, 17-19 nucleotides in length, 19-25 nucleotides in length, 19-23 nucleotides in length, 19-21 nucleotides in length, 21-25 nucleotides in length, or 21-23 nucleotides in length. Without limitations, the sense and antisense strands can be equal length or unequal length. For example, the sense strand and the antisense strand independently have a length of 18, 19, 20, 21, 22, 23, 24 or 25 nucleotides.

In some embodiments, the antisense strand is of length 15-35 nucleotides. In some embodiments, the antisense strand is 15-35, 17-35, 17-30, 25-35, 27-30, 17-23, 17-21, 17-19, 19-25, 19-23, 19-21, 21-25, 21-25, or 21-23 nucleotides in length. For example, the antisense strand can be 15, 16, 17, 18, 19, 20, 21, 22, 23, 24, 25, 26, 27, 28, 29, 30, 31, 32, 33, 34 or 35 nucleotides in length. In some embodiments, the antisense strand is 19, 20, 21, 22, 23, 24 or 25 nucleotides in length. For example, the antisense strand is 21, 22, 23, 24 or 25 nucleotides in length. In some particular embodiments, the antisense strand is 22, 23 or 24 nucleotides in length. For example, the antisense strand is 23 nucleotides in length.

Similar to the antisense strand, the sense strand can be, in some embodiments, 15-35 nucleotides in length. In some embodiments, the sense strand is 15-35, 17-35, 17-30, 25-35, 27-30, 17-23, 17-21, 17-19, 19-25, 19-23, 19-21, 21-25, 21-25, or 21-23 nucleotides in length. For example, the sense strand can be 15, 16, 17, 18, 19, 20, 21, 22, 23, 24, 25, 26, 27, 28, 29, 30, 31, 32, 33, 34 or 35 nucleotides in length. In some embodiments, the sense strand is 17, 18, 19, 20, 21, 22, 23, 24 or 25 nucleotides in length. For example, the sense strand is 19, 20, 21, 22 or 23 nucleotides in length. In some particular embodiments, the sense strand is 20, 21 or 22 nucleotides in length. For example, the sense strand is 21nucleotides in length

In some embodiments, the sense strand can be 15-35 nucleotides in length, and the antisense strand can be independent from the sense strand, 15-35 nucleotides in length. In some embodiments, the sense strand is 15-35, 17-35, 17-30, 25-35, 27-30, 17-23, 17-21, 17-19, 19-25, 19-23, 19-21, 21-25, 21-25, or 21-23 nucleotides in length, and the antisense strand is independently 15-35, 17-35, 17-30, 25-35, 27-30, 17-23, 17-21, 17-19, 19-25, 19-23, 19-21, 21-25, 21-25, or 21-23 nucleotides in length. For example, the sense and the antisense strand can be independently 15, 16, 17, 18, 19, 20, 21, 22, 23, 24, 25, 26, 27, 28, 29, 30, 31, 32, 33, 34 or 35 nucleotides in length. In some embodiments, the sense strand and the antisense strand are independently 17, 18, 19, 20, 21, 22, 23, 24 or 25 nucleotides in length. For example, the sense strand is 19, 20, 21, 22 or 23 nucleotides in length and the antisense strand is 21, 22, 23, 24 or 25 nucleotides in length. In some particular embodiments, the sense strand is 20, 21 or 22 nucleotides in length and the antisense strand is 22, 23 or 24 nucleotides in length. For example, the sense strand is 21 nucleotides in length and the antisense strand is 23 nucleotides in length.

The sense strand and antisense strand typically form a double-stranded or duplex region. Without limitations, the duplex region of a dsRNA agent described herein can be 12-35 nucleotide (or base) pairs in length. For example, the duplex region can be between 14-35 nucleotide pairs in length, 17-30 nucleotide pairs in length, 25-35 nucleotides in length, 27-35 nucleotide pairs in length, 17-23 nucleotide pairs in length, 17-21 nucleotide pairs in length, 17-19 nucleotide pairs in length, 19-25 nucleotide pairs in length, 19-23 nucleotide pairs in length, 19-21 nucleotide pairs in length, 21-25 nucleotide pairs in length, or 21-23 nucleotide pairs in length. In another example, the duplex region is selected from 15, 16, 17, 18, 19, 20, 21, 22, 23, 24, 25, 26, and 27 nucleotide pairs in length. In some embodiments, the duplex region is 18, 19, 20, 21, 22, 23, 24 or 25 nucleotide pairs in length. For example, the duplex region is 19, 20, 21, 22 or 23 nucleotide pairs in length. In some embodiments, the duplex region is 20, 21 or 22 nucleotide pairs in length. For example, the dsRNA molecule has a duplex region of 21 base pairs.

### Uses of Oligonucleotides

The oligonucleotides described herein can be used for any use know in the art for oligonucleotides. For example, the oligonucleotides described herein can be used in RNA interference based gene silencing techniques. Some exemplary uses for the oligonucleotides described herein include, but are not limited to, RNA interference agents, antisense oligonucleotides, aptamers, miRNAs, ribozymes, triplex forming oligonucleotides and the like.

Accordingly, in another aspect, the disclosure is directed to a use of an oligonucleotide and/or dsRNA molecule described herein for inhibiting expression of a target gene. In some embodiments, the present invention further relates to a use of an oligonucleotide and/or dsRNA molecule described herein for inhibiting expression of a target gene in vitro.

In another aspect, the disclosure is directed to a use of an oligonucleotide and/or dsRNA molecule described herein for use in inhibiting expression of a target gene in a subject. The subject may be any animal, such as a mammal, e.g., a mouse, a rat, a sheep, a cattle, a dog, a cat, or a human

In some embodiments, the oligonucleotide and/or dsRNA molecule described herein is administered in buffer.

In some embodiments, oligonucleotide and/or dsRNA molecule described herein described herein can be formulated for administration to a subject. A formulated oligonucleotide and/or dsRNA composition can assume a variety of states. In some examples, the composition is at least partially crystalline, uniformly crystalline, and/or anhydrous (e.g., less than 80, 50, 30, 20, or 10% water). In another example, the siRNA is in an aqueous phase, e.g., in a solution that includes water.

The aqueous phase or the crystalline compositions can, e.g., be incorporated into a delivery vehicle, e.g., a liposome (particularly for the aqueous phase) or a particle (e.g., a microparticle as can be appropriate for a crystalline composition). Generally, the siRNA composition is formulated in a manner that is compatible with the intended method of administration, as described herein. For example, in particular embodiments the composition is prepared by at least one of the following methods: spray drying, lyophilization, vacuum drying, evaporation, fluid bed drying, or a combination of these techniques; or sonication with a lipid, freeze-drying, condensation and other self-assembly.

A oligonucleotide and/or dsRNA preparation can be formulated in combination with another agent, e.g., another therapeutic agent or an agent that stabilizes an oligonucleotide and/or dsRNA, e.g., a protein that complexes with oligonucleotide and/or dsRNA. Still other agents include chelating agents, e.g., EDTA (e.g., to remove divalent cations such as Mg²⁺), salts, RNAse inhibitors (e.g., a broad specificity RNAse inhibitor such as RNAsin) and so forth.

In some embodiments, the oligonucleotide and/or dsRNA preparation includes another dsRNA compound, e.g., a second dsRNA that can mediate RNAi with respect to a second gene, or with respect to the same gene. Still other preparation can include at least 3, 5, ten, twenty, fifty, or a hundred or more different siRNA species. Such dsRNAs can mediate RNAi with respect to a similar number of different genes.

In some embodiments, the oligonucleotide and/or dsRNA preparation includes at least a second therapeutic agent (e.g., an agent other than a RNA or a DNA). For example, a oligonucleotide and/or dsRNA composition for the treatment of a viral disease, e.g., HIV, might include a known antiviral agent (e.g., a protease inhibitor or reverse transcriptase inhibitor). In another example, a dsRNA composition for the treatment of a cancer might further comprise a chemotherapeutic agent.

Exemplary formulations which can be used for administering the oligonucleotide and/or dsRNA according to the present invention are discussed below.

Liposomes. A oligonucleotide and/or dsRNA preparation can be formulated for delivery in a membranous molecular assembly, e.g., a liposome or a micelle. As used herein, the term “liposome” refers to a vesicle composed of amphiphilic lipids arranged in at least one bilayer, e.g., one bilayer or a plurality of bilayers. Liposomes include unilamellar and multilamellar vesicles that have a membrane formed from a lipophilic material and an aqueous interior. The aqueous portion contains the oligonucleotide and/or dsRNA composition. The lipophilic material isolates the aqueous interior from an aqueous exterior, which typically does not include the oligonucleotide and/or dsRNA composition, although in some examples, it may. Liposomes are useful for the transfer and delivery of active ingredients to the site of action. Because the liposomal membrane is structurally similar to biological membranes, when liposomes are applied to a tissue, the liposomal bilayer fuses with bilayer of the cellular membranes. As the merging of the liposome and cell progresses, the internal aqueous contents that include the oligonucleotide and/or dsRNA are delivered into the cell where the dsRNA can specifically bind to a target RNA and can mediate RNAi. In some embodiments, the liposomes are also specifically targeted, e.g., to direct the oligonucleotide and/or dsRNA to particular cell types.

A liposome containing oligonucleotide and/or dsRNA can be prepared by a variety of methods. In one example, the lipid component of a liposome is dissolved in a detergent so that micelles are formed with the lipid component. For example, the lipid component can be an amphipathic cationic lipid or lipid conjugate. The detergent can have a high critical micelle concentration and may be nonionic. Exemplary detergents include cholate, CHAPS, octylglucoside, deoxycholate, and lauroyl sarcosine. The dsRNA preparation is then added to the micelles that include the lipid component. The cationic groups on the lipid interact with the siRNA and condense around the dsRNA to form a liposome. After condensation, the detergent is removed, e.g., by dialysis, to yield a liposomal preparation of oligonucleotide and/or dsRNA.

If necessary a carrier compound that assists in condensation can be added during the condensation reaction, e.g., by controlled addition. For example, the carrier compound can be a polymer other than a nucleic acid (e.g., spermine or spermidine). pH can also be adjusted to favor condensation.

Further description of methods for producing stable polynucleotide delivery vehicles, which incorporate a polynucleotide/cationic lipid complex as structural components of the delivery vehicle, are described in, e.g., WO 96/37194. Liposome formation can also include one or more aspects of exemplary methods described in Felgner, P. L. et al., Proc. Nad. Acad. Sci., USA 8:7413-7417, 1987; U.S. Pat. Nos. 4,897,355; 5,171,678; Bangham, et al. M. Mol. Biol. 23:238, 1965; Olson, et al. Biochim. Biophys. Acta 557:9, 1979; Szoka, et al. Proc. Natl. Acad. Sci. 75: 4194, 1978; Mayhew, et al. Biochim. Biophys. Acta 775:169, 1984; Kim, et al. Biochim. Biophys. Acta 728:339, 1983; and Fukunaga, et al. Endocrinol. 115:757, 1984, which are incorporated by reference in their entirety. Commonly used techniques for preparing lipid aggregates of appropriate size for use as delivery vehicles include sonication and freeze-thaw plus extrusion (see, e.g., Mayer, et al. Biochim. Biophys. Acta 858:161, 1986, which is incorporated by reference in its entirety). Microfluidization can be used when consistently small (50 to 200 nm) and relatively uniform aggregates are desired (Mayhew, et al. Biochim. Biophys. Acta 775:169, 1984, which is incorporated by reference in its entirety). These methods are readily adapted to packaging oligonucleotide and/or dsRNA preparations into liposomes.

Liposomes that are pH-sensitive or negatively-charged entrap nucleic acid molecules rather than complex with them. Since both the nucleic acid molecules and the lipid are similarly charged, repulsion rather than complex formation occurs. Nevertheless, some nucleic acid molecules are entrapped within the aqueous interior of these liposomes. pH-sensitive liposomes have been used to deliver DNA encoding the thymidine kinase gene to cell monolayers in culture. Expression of the exogenous gene was detected in the target cells (Zhou et al., Journal of Controlled Release, 19, (1992) 269-274, which is incorporated by reference in its entirety).

One major type of liposomal composition includes phospholipids other than naturally-derived phosphatidylcholine. Neutral liposome compositions, for example, can be formed from dimyristoyl phosphatidylcholine (DMPC) or dipalmitoyl phosphatidylcholine (DPPC). Anionic liposome compositions generally are formed from dimyristoyl phosphatidylglycerol, while anionic fusogenic liposomes are formed primarily from dioleoyl phosphatidylethanolamine (DOPE). Another type of liposomal composition is formed from phosphatidylcholine (PC) such as, for example, soybean PC, and egg PC. Another type is formed from mixtures of phospholipid and/or phosphatidylcholine and/or cholesterol.

Examples of other methods to introduce liposomes into cells in vitro and include U.S. Pat. Nos. 5,283,185; 5,171,678; WO 94/00569; WO 93/24640; WO 91/16024; Felgner, J. Biol. Chem. 269:2550, 1994; Nabel, Proc. Natl. Acad. Sci. 90:11307, 1993; Nabel, Human Gene Ther. 3:649, 1992; Gershon, Biochem. 32:7143, 1993; and Strauss EMBO J. 11:417, 1992.

In some embodiments, cationic liposomes are used. Cationic liposomes possess the advantage of being able to fuse to the cell membrane. Non-cationic liposomes, although not able to fuse as efficiently with the plasma membrane, are taken up by macrophages in vivo and can be used to deliver siRNAs to macrophages.

Further advantages of liposomes include: liposomes obtained from natural phospholipids are biocompatible and biodegradable; liposomes can incorporate a wide range of water and lipid soluble drugs; liposomes can protect encapsulated siRNAs in their internal compartments from metabolism and degradation (Rosoff, in “Pharmaceutical Dosage Forms,” Lieberman, Rieger and Banker (Eds.), 1988, volume 1, p. 245). Important considerations in the preparation of liposome formulations are the lipid surface charge, vesicle size and the aqueous volume of the liposomes.

A positively charged synthetic cationic lipid, N-[1-(2,3-dioleyloxy)propyl]-N,N,N-trimethylammonium chloride (DOTMA) can be used to form small liposomes that interact spontaneously with nucleic acid to form lipid-nucleic acid complexes which are capable of fusing with the negatively charged lipids of the cell membranes of tissue culture cells, resulting in delivery of siRNA (see, e.g., Felgner, P. L. et al., Proc. Natl. Acad. Sci., USA 8:7413-7417, 1987 and U.S. Pat. No. 4,897,355 for a description of DOTMA and its use with DNA, which are incorporated by reference in their entirety).

A DOTMA analogue, 1,2-bis(oleoyloxy)-3-(trimethylammonia)propane (DOTAP) can be used in combination with a phospholipid to form DNA-complexing vesicles. Lipofectin™ Bethesda Research Laboratories, Gaithersburg, Md.) is an effective agent for the delivery of highly anionic nucleic acids into living tissue culture cells that comprise positively charged DOTMA liposomes which interact spontaneously with negatively charged polynucleotides to form complexes. When enough positively charged liposomes are used, the net charge on the resulting complexes is also positive. Positively charged complexes prepared in this way spontaneously attach to negatively charged cell surfaces, fuse with the plasma membrane, and efficiently deliver functional nucleic acids into, for example, tissue culture cells. Another commercially available cationic lipid, 1,2-bis(oleoyloxy)-3,3-(trimethylammonia)propane (“DOTAP”) (Boehringer Mannheim, Indianapolis, Indiana) differs from DOTMA in that the oleoyl moieties are linked by ester, rather than ether linkages.

Other reported cationic lipid compounds include those that have been conjugated to a variety of moieties including, for example, carboxyspermine which has been conjugated to one of two types of lipids and includes compounds such as 5-carboxyspermylglycine dioctaoleoylamide (“DOGS”) (Transfectam™, Promega, Madison, Wisconsin) and dipalmitoylphosphatidylethanolamine 5-carboxyspermyl-amide (“DPPES”) (see, e.g., U.S. Pat. No. 5,171,678).

Another cationic lipid conjugate includes derivatization of the lipid with cholesterol (“DC-Chol”) which has been formulated into liposomes in combination with DOPE (See, Gao, X. and Huang, L., Biochim. Biophys. Res. Commun. 179:280, 1991). Lipopolylysine, made by conjugating polylysine to DOPE, has been reported to be effective for transfection in the presence of serum (Zhou, X. et al., Biochim. Biophys. Acta 1065:8, 1991, which is incorporated by reference in its entirety). For certain cell lines, these liposomes containing conjugated cationic lipids, are said to exhibit lower toxicity and provide more efficient transfection than the DOTMA-containing compositions. Other commercially available cationic lipid products include DMRIE and DMRIE-HP (Vical, La Jolla, California) and Lipofectamine (DOSPA) (Life Technology, Inc., Gaithersburg, Maryland). Other cationic lipids suitable for the delivery of oligonucleotides are described in WO 98/39359 and WO 96/37194.

Liposomal formulations are particularly suited for topical administration. Liposomes present several advantages over other formulations. Such advantages include reduced side effects related to high systemic absorption of the administered drug, increased accumulation of the administered drug at the desired target, and the ability to administer siRNA, into the skin. In some implementations, liposomes are used for delivering siRNA to epidermal cells and also to enhance the penetration of siRNA into dermal tissues, e.g., into skin. For example, the liposomes can be applied topically. Topical delivery of drugs formulated as liposomes to the skin has been documented (see, e.g., Weiner et al., Journal of Drug Targeting, 1992, vol. 2,405-410 and du Plessis et al., Antiviral Research, 18, 1992, 259-265; Mannino, R. J. and Fould-Fogerite, S., Biotechniques 6:682-690, 1988; Itani, T. et al. Gene 56:267-276. 1987; Nicolau, C. et al. Meth. Enz. 149:157-176, 1987; Straubinger, R. M. and Papahadjopoulos, D. Meth. Enz. 101:512-527, 1983; Wang, C. Y. and Huang, L., Proc. Natl. Acad. Sci. USA 84:7851-7855, 1987, which are incorporated by reference in their entirety).

Non-ionic liposomal systems have also been examined to determine their utility in the delivery of drugs to the skin, in particular systems comprising non-ionic surfactant and cholesterol. Non-ionic liposomal formulations comprising Novasome I (glyceryl dilaurate/cholesterol/polyoxyethylene-10-stearyl ether) and Novasome II (glyceryl distearate/cholesterol/polyoxyethylene-10-stearyl ether) were used to deliver a drug into the dermis of mouse skin. Such formulations with dsRNA described herein are useful for treating a dermatological disorder.

Liposomes that include oligonucleotide and/or dsRNA described herein can be made highly deformable. Such deformability can enable the liposomes to penetrate through pore that are smaller than the average radius of the liposome. For example, transfersomes are a type of deformable liposomes. Transfersomes can be made by adding surface edge activators, usually surfactants, to a standard liposomal composition. Transfersomes that include oligonucleotide and/or dsRNA described herein can be delivered, for example, subcutaneously by infection in order to deliver dsRNA to keratinocytes in the skin. In order to cross intact mammalian skin, lipid vesicles must pass through a series of fine pores, each with a diameter less than 50 nm, under the influence of a suitable transdermal gradient. In addition, due to the lipid properties, these transfersomes can be self-optimizing (adaptive to the shape of pores, e.g., in the skin), self-repairing, and can frequently reach their targets without fragmenting, and often self-loading.

Other formulations amenable to the present invention are described in U.S. provisional application Ser. No. 61/018,616, filed Jan. 2, 2008; 61/018,611, filed Jan. 2, 2008; 61/039,748, filed Mar. 26, 2008; 61/047,087, filed Apr. 22, 2008 and 61/051,528, filed May 8, 2008. PCT application no PCT/US2007/080331, filed Oct. 3, 2007 also describes formulations that are amenable to the present invention.

Surfactants. The oligonucleotide and/or dsRNA compositions can include a surfactant. In some embodiments, the dsRNA is formulated as an emulsion that includes a surfactant. The most common way of classifying and ranking the properties of the many different types of surfactants, both natural and synthetic, is by the use of the hydrophile/lipophile balance (HLB). The nature of the hydrophilic group provides the most useful means for categorizing the different surfactants used in formulations (Rieger, in “Pharmaceutical Dosage Forms,” Marcel Dekker, Inc., New York, NY, 1988, p. 285).

If the surfactant molecule is not ionized, it is classified as a nonionic surfactant. Nonionic surfactants find wide application in pharmaceutical products and are usable over a wide range of pH values. In general, their HLB values range from 2 to about 18 depending on their structure. Nonionic surfactants include nonionic esters such as ethylene glycol esters, propylene glycol esters, glyceryl esters, polyglyceryl esters, sorbitan esters, sucrose esters, and ethoxylated esters. Nonionic alkanolamides and ethers such as fatty alcohol ethoxylates, propoxylated alcohols, and ethoxylated/propoxylated block polymers are also included in this class. The polyoxyethylene surfactants are the most popular members of the nonionic surfactant class.

If the surfactant molecule carries a negative charge when it is dissolved or dispersed in water, the surfactant is classified as anionic. Anionic surfactants include carboxylates such as soaps, acyl lactylates, acyl amides of amino acids, esters of sulfuric acid such as alkyl sulfates and ethoxylated alkyl sulfates, sulfonates such as alkyl benzene sulfonates, acyl isethionates, acyl taurates and sulfosuccinates, and phosphates. The most important members of the anionic surfactant class are the alkyl sulfates and the soaps.

If the surfactant molecule carries a positive charge when it is dissolved or dispersed in water, the surfactant is classified as cationic. Cationic surfactants include quaternary ammonium salts and ethoxylated amines. The quaternary ammonium salts are the most used members of this class.

If the surfactant molecule has the ability to carry either a positive or negative charge, the surfactant is classified as amphoteric. Amphoteric surfactants include acrylic acid derivatives, substituted alkylamides, N-alkylbetaines and phosphatides.

The use of surfactants in drug products, formulations and in emulsions has been reviewed (Rieger, in “Pharmaceutical Dosage Forms,” Marcel Dekker, Inc., New York, NY, 1988, p. 285).

Micelles and other Membranous Formulations. “Micelles” are defined herein as a particular type of molecular assembly in which amphipathic molecules are arranged in a spherical structure such that all the hydrophobic portions of the molecules are directed inward, leaving the hydrophilic portions in contact with the surrounding aqueous phase. The converse arrangement exists if the environment is hydrophobic.

A mixed micellar formulation suitable for delivery through transdermal membranes may be prepared by mixing an aqueous solution of the oligonucleotide and/or dsRNA composition, an alkali metal C₈ to C₂₂ alkyl sulphate, and a micelle forming compounds. Exemplary micelle forming compounds include lecithin, hyaluronic acid, pharmaceutically acceptable salts of hyaluronic acid, glycolic acid, lactic acid, chamomile extract, cucumber extract, oleic acid, linoleic acid, linolenic acid, monoolein, monooleates, monolaurates, borage oil, evening of primrose oil, menthol, trihydroxy oxo cholanyl glycine and pharmaceutically acceptable salts thereof, glycerin, polyglycerin, lysine, polylysine, triolein, polyoxyethylene ethers and analogues thereof, polidocanol alkyl ethers and analogues thereof, chenodeoxycholate, deoxycholate, and mixtures thereof. The micelle forming compounds may be added at the same time or after addition of the alkali metal alkyl sulphate. Mixed micelles will form with substantially any kind of mixing of the ingredients but vigorous mixing in order to provide smaller size micelles.

In one method, a first micellar composition is prepared which contains the oligonucleotide and/or dsRNA composition and at least the alkali metal alkyl sulphate. The first micellar composition is then mixed with at least three micelle forming compounds to form a mixed micellar composition. In another method, the micellar composition is prepared by mixing the dsRNA composition, the alkali metal alkyl sulphate and at least one of the micelle forming compounds, followed by addition of the remaining micelle forming compounds, with vigorous mixing.

Phenol and/or m-cresol may be added to the mixed micellar composition to stabilize the formulation and protect against bacterial growth. Alternatively, phenol and/or m-cresol may be added with the micelle forming ingredients. An isotonic agent such as glycerin may also be added after formation of the mixed micellar composition.

For delivery of the micellar formulation as a spray, the formulation can be put into an aerosol dispenser and the dispenser is charged with a propellant. The propellant, which is under pressure, is in liquid form in the dispenser. The ratios of the ingredients are adjusted so that the aqueous and propellant phases become one, i.e., there is one phase. If there are two phases, it is necessary to shake the dispenser prior to dispensing a portion of the contents, e.g., through a metered valve. The dispensed dose of pharmaceutical agent is propelled from the metered valve in a fine spray.

Propellants may include hydrogen-containing chlorofluorocarbons, hydrogen-containing fluorocarbons, dimethyl ether and diethyl ether. In certain embodiments, HFA 134a (1,1,1,2 tetrafluoroethane) may be used.

The specific concentrations of the essential ingredients can be determined by relatively straightforward experimentation. For absorption through the oral cavities, it is often desirable to increase, e.g., at least double or triple, the dosage for through injection or administration through the gastrointestinal tract.

Particles. In some embodiments, dsRNA preparations can be incorporated into a particle, e.g., a microparticle. Microparticles can be produced by spray-drying, but may also be produced by other methods including lyophilization, evaporation, fluid bed drying, vacuum drying, or a combination of these techniques.

### Pharmaceutical Compositions

The oligonucleotide and/or dsRNA described herein can be formulated for pharmaceutical use. The present invention further relates to a pharmaceutical composition comprising the oligonucleotide and/or dsRNA described herein. Pharmaceutically acceptable compositions comprise a therapeutically-effective amount of one or more of the dsRNA molecules in any of the preceding embodiments, taken alone or formulated together with one or more pharmaceutically acceptable carriers (additives), excipient and/or diluents.

The pharmaceutical compositions may be specially formulated for administration in solid or liquid form, including those adapted for the following: (1) oral administration, for example, drenches (aqueous or non-aqueous solutions or suspensions), tablets, e.g., those targeted for buccal, sublingual, and systemic absorption, boluses, powders, granules, pastes for application to the tongue; (2) parenteral administration, for example, by subcutaneous, intramuscular, intravenous or epidural injection as, for example, a sterile solution or suspension, or sustained-release formulation; (3) topical application, for example, as a cream, ointment, or a controlled-release patch or spray applied to the skin; (4) intravaginally or intrarectally, for example, as a pessary, cream or foam; (5) sublingually; (6) ocularly; (7) transdermally; or (8) nasally. Delivery using subcutaneous or intravenous methods can be particularly advantageous.

The phrase “therapeutically-effective amount” as used herein means that amount of a compound, material, or composition comprising a dsRNA molecule described herein which is effective for producing some desired therapeutic effect in at least a sub-population of cells in an animal at a reasonable benefit/risk ratio applicable to any medical treatment.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically-acceptable carrier” as used herein means a pharmaceutically-acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) lubricating agents, such as magnesium state, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; and (22) other non-toxic compatible substances employed in pharmaceutical formulations.

The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient which can be combined with a carrier material to produce a single dosage form will generally be that amount of the compound which produces a therapeutic effect. Generally, out of one hundred percent, this amount will range from about 0.1 percent to about ninety-nine percent of active ingredient, preferably from about 5 percent to about 70 percent, most preferably from about 10 percent to about 30 percent.

In certain embodiments, a formulation of the present invention comprises an excipient selected from the group consisting of cyclodextrins, celluloses, liposomes, micelle forming agents, e.g., bile acids, and polymeric carriers, e.g., polyesters and polyanhydrides; and a compound of the present invention. In certain embodiments, an aforementioned formulation renders orally bioavailable a compound of the present invention.

Methods of preparing these formulations or compositions include the step of bringing into association an oligonucleotide and/or dsRNA with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally-administered drug form is accomplished by dissolving or suspending the drug in an oil vehicle.

The oligonucleotide and/or dsRNA described herein may be formulated for administration in any convenient way for use in human or veterinary medicine, by analogy with other pharmaceuticals.

The term “treatment” is intended to encompass therapy and cure. The patient receiving this treatment is any animal in need, including primates, in particular humans, and other mammals such as equines, cattle, swine and sheep; and poultry and pets in general.

The oligonucleotide and/or dsRNA described herein or a pharmaceutical composition comprising an oligonucleotide and/or dsRNA described herein can be administered to a subject using different routes of delivery. A composition that includes an oligonucleotide and/or dsRNA described herein described herein can be delivered to a subject by a variety of routes. Exemplary routes include: intravenous, subcutaneous, topical, rectal, anal, vaginal, nasal, pulmonary, ocular.

The oligonucleotide and/or dsRNA described herein may be administered in a number of ways depending upon whether local or systemic treatment is desired and upon the area to be treated. Administration may be topical (including ophthalmic, vaginal, rectal, intranasal, transdermal), oral or parenteral. Parenteral administration includes intravenous drip, subcutaneous, intraperitoneal or intramuscular injection, or intrathecal or intraventricular administration.

The route and site of administration may be chosen to enhance targeting. For example, to target muscle cells, intramuscular injection into the muscles of interest would be a logical choice. Lung cells might be targeted by administering the oligonucleotide and/or dsRNA described herein in aerosol form. The vascular endothelial cells could be targeted by coating a balloon catheter with the oligonucleotide and/or dsRNA described herein and mechanically introducing the oligonucleotide and/or dsRNA described herein.

In one aspect, provided herein is a method of administering an oligonucleotide and/or dsRNA described herein, to a subject (e.g., a human subject). In another aspect, the present invention relates to an oligonucleotide and/or dsRNA described herein for use in inhibiting expression of a target gene in a subject. The method or the medical use includes administering a unit dose of the oligonucleotide and/or dsRNA described herein. In some embodiments, the unit dose is less than 10 mg per kg of bodyweight, or less than 10, 5, 2, 1, 0.5, 0.1, 0.05, 0.01, 0.005, 0.001, 0.0005, 0.0001, 0.00005 or 0.00001 mg per kg of bodyweight, and less than 200 nmole of RNA agent (e.g., about 4.4×10¹⁶ copies) per kg of bodyweight, or less than 1500, 750, 300, 150, 75, 15, 7.5, 1.5, 0.75, 0.15, 0.075, 0.015, 0.0075, 0.0015, 0.00075, 0.00015 nmole of oligonucleotide and/or dsRNA described herein per kg of bodyweight.

The defined amount can be an amount effective to treat or prevent a disease or disorder, e.g., a disease or disorder associated with the target gene. The unit dose, for example, can be administered by injection (e.g., intravenous, subcutaneous or intramuscular), an inhaled dose, or a topical application. In some embodiments dosages may be less than 10, 5, 2, 1, or 0.1 mg/kg of body weight.

In some embodiments, the unit dose is administered less frequently than once a day, e.g., less than every 2, 4, 8 or 30 days. In another embodiment, the unit dose is not administered with a frequency (e.g., not a regular frequency). For example, the unit dose may be administered a single time.

In some embodiments, the effective dose is administered with other traditional therapeutic modalities.

In some embodiments, a subject is administered an initial dose and one or more maintenance doses. The maintenance dose or doses can be the same or lower than the initial dose, e.g., one-half less of the initial dose. A maintenance regimen can include treating the subject with a dose or doses ranging from 0.01 μg to 15 mg/kg of body weight per day, e.g., 10, 1, 0.1, 0.01, 0.001, or 0.00001 mg per kg of bodyweight per day. The maintenance doses are, for example, administered no more than once every 2, 5, 10, or 30 days. Further, the treatment regimen may last for a period of time which will vary depending upon the nature of the particular disease, its severity and the overall condition of the patient. In certain embodiments the dosage may be delivered no more than once per day, e.g., no more than once per 24, 36, 48, or more hours, e.g., no more than once for every 5 or 8 days. Following treatment, the patient can be monitored for changes in his condition and for alleviation of the symptoms of the disease state. The dosage of the compound may either be increased in the event the patient does not respond significantly to current dosage levels, or the dose may be decreased if an alleviation of the symptoms of the disease state is observed, if the disease state has been ablated, or if undesired side-effects are observed.

The effective dose can be administered in a single dose or in two or more doses, as desired or considered appropriate under the specific circumstances. If desired to facilitate repeated or frequent infusions, implantation of a delivery device, e.g., a pump, semi-permanent stent (e.g., intravenous, intraperitoneal, intracisternal or intracapsular), or reservoir may be advisable.

In some embodiments, the composition includes a plurality of dsRNA molecule species. In another embodiment, the dsRNA molecule species has sequences that are non-overlapping and non-adjacent to another species with respect to a naturally occurring target sequence. In another embodiment, the plurality of dsRNA molecule species is specific for different naturally occurring target genes. In another embodiment, the dsRNA molecule is allele specific.

The oligonucleotide and/or dsRNA described herein can be administered to mammals, particularly large mammals such as nonhuman primates or humans in a number of ways.

In some embodiments, the administration of the oligonucleotide and/or dsRNA composition described herein is parenteral, e.g., intravenous (e.g., as a bolus or as a diffusible infusion), intradermal, intraperitoneal, intramuscular, intrathecal, intraventricular, intracranial, subcutaneous, transmucosal, buccal, sublingual, endoscopic, rectal, oral, vaginal, topical, pulmonary, intranasal, urethral or ocular. Administration can be provided by the subject or by another person, e.g., a health care provider. The medication can be provided in measured doses or in a dispenser which delivers a metered dose. Selected modes of delivery are discussed in more detail below.

The invention provides methods, compositions, and kits, for rectal administration or delivery of oligonucleotide and/or dsRNA composition described herein.

### Methods of Inhibiting Expression of a Target Gene

Aspects of the disclosure also relate to methods for inhibiting the expression of a target gene. The method comprises administering to the subject in an amount sufficient to inhibit expression of the target gene: (II) a double-stranded RNA described herein, where the wherein the first strand is complementary to a target gene; and/or (ii) an oligonucleotide described herein, wherein the oligonucleotide is complementary to a target gene.

The present disclosure further relates to a use of an oligonucleotide and/or dsRNA molecule described herein for inhibiting expression of a target gene in a target cell. The present disclosure further relates to a use of an oligonucleotide and/or dsRNA molecule described herein for inhibiting expression of a target gene in a target cell in vitro.

Another aspect the invention relates to a method of modulating the expression of a target gene in a cell, comprising administering to said cell an oligonucleotide and/or dsRNA molecule described herein. In some embodiments, the target gene is selected from the group consisting of Factor VII, Eg5, PCSK9, TPX2, apoB, SAA, TTR, RSV, PDGF beta gene, Erb-B gene, Src gene, CRK gene, GRB2 gene, RAS gene, MEKK gene, INK gene, RAF gene, Erk1/2 gene, PCNA(p21) gene, MYB gene, JUN gene, FOS gene, BCL-2 gene, hepcidin, Activated Protein C, Cyclin D gene, VEGF gene, EGFR gene, Cyclin A gene, Cyclin E gene, WNT-1 gene, beta-catenin gene, c-MET gene, PKC gene, NFKB gene, STAT3 gene, survivin gene, Her2/Neu gene, topoisomerase I gene, topoisomerase II alpha gene, mutations in the p73 gene, mutations in the p21(WAFT/CIP1) gene, mutations in the p27(KIP1) gene, mutations in the PPM1D gene, mutations in the RAS gene, mutations in the caveolin I gene, mutations in the MIB I gene, mutations in the MTAI gene, mutations in the M68 gene, mutations in tumor suppressor genes, and mutations in the p53 tumor suppressor gene.

### Some Selected Definitions

For convenience, certain terms employed herein, in the specification, examples and appended claims are collected herein. Unless stated otherwise, or implicit from context, the following terms and phrases include the meanings provided below Unless explicitly stated otherwise, or apparent from context, the terms and phrases below do not exclude the meaning that the term or phrase has acquired in the art to which it pertains. The definitions are provided to aid in describing particular embodiments, and are not intended to limit the claimed invention, because the scope of the invention is limited only by the claims. Further, unless otherwise required by context, singular terms shall include pluralities and plural terms shall include the singular.

Unless defined otherwise, all technical and scientific terms used herein have the same meaning as those commonly understood to one of ordinary skill in the art to which this invention pertains. Although any known methods, devices, and materials may be used in the practice or testing of the invention, the methods, devices, and materials in this regard are described herein.

Further, the practice of the present invention can employ, unless otherwise indicated, conventional techniques of molecular biology (including recombinant techniques), microbiology, cell biology, biochemistry, and immunology, which are within the skill of the art. Such techniques are explained fully in the literature, such as, “Molecular Cloning: A Laboratory Manual”, second edition (Sambrook et al., 1989); “Oligonucleotide Synthesis” (M. J. Gait, ed., 1984); “Animal Cell Culture” (R. I. Freshney, ed., 1987); “Methods in Enzymology” (Academic Press, Inc.); “Current Protocols in Molecular Biology” (F. M. Ausubel et al., eds., 1987, and periodic updates); “PCR: The Polymerase Chain Reaction”, (Mullis et al., ed., 1994); “A Practical Guide to Molecular Cloning” (Perbal Bernard V., 1988); “Phage Display: A Laboratory Manual” (Barbas et al., 2001).

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

As used herein the term “comprising” or “comprises” is used in reference to compositions, methods, and respective component(s) thereof, that are essential to the invention, yet open to the inclusion of unspecified elements, whether essential or not.

The singular terms “a,” “an,” and “the” include plural referents unless context clearly indicates otherwise. Similarly, the word “or” is intended to include “and” unless the context clearly indicates otherwise. It is further noted that the claims can be drafted to exclude any optional element. As such, this statement is intended to serve as antecedent basis for use of such exclusive terminology as “solely,” “only” and the like in connection with the recitation of claim elements, or use of a “negative” limitation.

As used herein, the term “alkyl” refers to a saturated hydrocarbon group which can be straight or branched having 1 to about 60 carbon atoms in the chain, and which preferably have about 6 to about 50 carbons in the chain. “Lower alkyl” refers to an alkyl group having 1 to about 8 carbon atoms. “Higher alkyl” refers to an alkyl group having about 10 to about 20 carbon atoms. The alkyl group can be optionally substituted with one or more alkyl group substituents which can be the same or different, where “alkyl group substituent” includes halo, amino, aryl, hydroxyl, alkoxy, aryloxy, alkyloxy, alkylthio, arylthio, aralkyloxy, aralkylthio, carboxy, alkoxycarbonyl, oxo and cycloalkyl. “Branched” refers to an alkyl group in which a lower alkyl group, such as methyl, ethyl or propyl, is attached to a linear alkyl chain. Exemplary alkyl groups include methyl, ethyl, propyl, i-propyl, n-butyl, t-butyl, n-pentyl, hexyl, heptyl, octyl, decyl, dodecyl, tridecyl, tetradecyl, pentadecyl and hexadecyl. Useful alkyl groups include branched or straight chain alkyl groups of 6 to 50 carbon, and also include the lower alkyl groups of 1 to about 4 carbons and the higher alkyl groups of about 12 to about 16 carbons.

As used herein, the term “aliphatic” refers to an alkyl, alkenyl, alkynyl, or alkenynyl group containing the referenced number of carbons, each as defined herein.

A “heteroalkyl” group substitutes any one of the carbons of the alkyl group with a heteroatom having the appropriate number of hydrogen atoms attached (e.g., a CH₂ group to an NH group or an O group). The term “heteroalkyl” include optionally substituted alkyl, alkenyl and alkynyl radicals which have one or more skeletal chain atoms selected from an atom other than carbon, e.g., oxygen, nitrogen, sulfur, phosphorus, silicon, or combinations thereof. In certain embodiments, the heteroatom(s) is placed at any interior position of the heteroalkyl group. Examples include, but are not limited to, —CH₂—O—CH₃, —CH₂—CH₂—O—CH₃, —CH₂—NH—CH₃, —CH₂—CH₂—NH—CH₃, —CH₂—N(CH₃)—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CL)₃, —CH₂—CH═N—OCH₃, and —CH═CH—N(CH₃)—CH₃. In some embodiments, up to two heteroatoms are consecutive, such as, by way of example, —CH₂—NH—OCH₃ and —CH₂—O—Si(CH₃)₃

As used herein, the term “alkenyl” refers to an unsaturated hydrocarbon group containing at least one carbon-carbon double bond. The alkenyl group can be optionally substituted with one or more “alkyl group substituents.” Exemplary alkenyl groups include vinyl, allyl, n-pentenyl, decenyl, dodecenyl, tetradecadienyl, heptadec-8-en-1-yl and heptadec-8,11-dien-1-yl.

As used herein, the term “alkynyl” refers to an unsaturated hydrocarbon group containing at least one carbon-carbon triple bond. The alkynyl group can be optionally substituted with one or more “alkyl group substituents.” Exemplary alkynyl groups include ethynyl, propargyl, n-pentynyl, decynyl and dodecynyl. Useful alkynyl groups include the lower alkynyl groups.

As used herein, the term “alkenynyl” refers to an unsaturated hydrocarbon group containing at least one carbon-carbon double bond and at least one carbon-carbon triple bond. In certain embodiments, a double bond and a triple bond within the alkenynyl group are conjugated, e.g.,

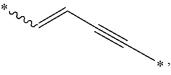

where the * represents a bond to the remainder of the alkenynyl group.

As used herein, the term “cycloalkyl” refers to a non-aromatic mono- or multicyclic ring system of about 3 to about 12 carbon atoms. The cycloalkyl group can be optionally partially unsaturated. The cycloalkyl group can be also optionally substituted with an aryl group substituent, oxo and/or alkylene. Representative monocyclic cycloalkyl rings include cyclopentyl, cyclohexyl and cycloheptyl. Useful multicyclic cycloalkyl rings include adamantyl, octahydronaphthyl, decalin, camphor, camphane, and noradamantyl.

“Heterocyclyl” refers to a nonaromatic 3-8 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively). Cₓheterocyclyl and Cₓ-C_{y}heterocyclyl are typically used where X and Y indicate the number of carbon atoms in the ring system. In some embodiments, 1, 2 or 3 hydrogen atoms of each ring can be substituted by a substituent. Exemplary heterocyclyl groups include, but are not limited to piperazinyl, pyrrolidinyl, dioxanyl, morpholinyl, tetrahydrofuranyl, piperidyl, 4-morpholyl, 4-piperazinyl, pyrrolidinyl, perhydropyrrolizinyl, 1,4-diazaperhydroepinyl, 1,3-dioxanyl, 1,4-dioxanyl and the like.

“Aryl” refers to an aromatic carbocyclic radical containing about 3 to about 13 carbon atoms. The aryl group can be optionally substituted with one or more aryl group substituents, which can be the same or different, where “aryl group substituent” includes alkyl, alkenyl, alkynyl, aryl, aralkyl, hydroxyl, alkoxy, aryloxy, aralkoxy, carboxy, aroyl, halo, nitro, trihalomethyl, cyano, alkoxycarbonyl, aryloxycarbonyl, aralkoxycarbonyl, acyloxy, acylamino, aroylamino, carbamoyl, alkylcarbamoyl, dialkylcarbamoyl, rylthio, alkylthio, alkylene and NRR′, where R and R′ are each independently hydrogen, alkyl, aryl and aralkyl. Exemplary aryl groups include substituted or unsubstituted phenyl and substituted or unsubstituted naphthyl.

“Heteroaryl” refers to an aromatic 3-8 membered monocyclic, 8-12 membered fused bicyclic, or 11-14 membered fused tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively.

Exemplary aryl and heteroaryls include, but are not limited to, phenyl, pyridinyl, pyrimidinyl, furanyl, thienyl, imidazolyl, thiazolyl, pyrazolyl, pyridazinyl, pyrazinyl, triazinyl, tetrazolyl, indolyl, benzyl, naphthyl, anthracenyl, azulenyl, fluorenyl, indanyl, indenyl, naphthyl, tetrahydronaphthyl, benzimidazolyl, benzofuranyl, benzothiofuranyl, benzothiophenyl, benzoxazolyl, benzoxazolinyl, benzthiazolyl, benztriazolyl, benztetrazolyl, benzisoxazolyl, benzisothiazolyl, benzimidazolinyl, carbazolyl, 4aH carbazolyl, carbolinyl, chromanyl, chromenyl, cinnolinyl, decahydroquinolinyl, 2H,6H-1,5,2-dithiazinyl, dihydrofuro[2,3 b]tetrahydrofuran, furanyl, furazanyl, imidazolidinyl, imidazolinyl, imidazolyl, 1H-indazolyl, indolenyl, indolinyl, indolizinyl, indolyl, 3H-indolyl, isatinoyl, isobenzofuranyl, isochromanyl, isoindazolyl, isoindolinyl, isoindolyl, isoquinolinyl, isothiazolyl, isoxazolyl, methylenedioxyphenyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxindolyl, pyrimidinyl, phenanthridinyl, phenanthrolinyl, phenazinyl, phenothiazinyl, phenoxathinyl, phenoxazinyl, phthalazinyl, piperazinyl, piperidinyl, piperidonyl, 4-piperidonyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl and xanthenyl, and the like. In some embodiments, 1, 2, 3, or 4 hydrogen atoms of each ring can be substituted by a substituent.

As used herein, the term “halogen” or “halo” refers to an atom selected from fluorine, chlorine, bromine and iodine. The term “halogen radioisotope” or “halo isotope” refers to a radionuclide of an atom selected from fluorine, chlorine, bromine and iodine.

A “halogen-substituted moiety” or “halo-substituted moiety”, as an isolated group or part of a larger group, means an aliphatic, alicyclic, or aromatic moiety, as described herein, substituted by one or more “halo” atoms, as such terms are defined in this application.

The term “haloalkyl” as used herein refers to alkyl and alkoxy structures structure with at least one substituent of fluorine, chorine, bromine or iodine, or with combinations thereof. In embodiments, where more than one halogen is included in the group, the halogens are the same or they are different. The terms “fluoroalkyl” and “fluoroalkoxy” include haloalkyl and haloalkoxy groups, respectively, in which the halo is fluorine. Exemplary halo-substituted alkyl includes haloalkyl, dihaloalkyl, trihaloalkyl, perhaloalkyl and the like (e.g. halosubstituted (C₁-C₃)alkyl includes chloromethyl, dichloromethyl, difluoromethyl, trifluoromethyl (CF₃), perfluoroethyl, 2,2,2-trifluoroethyl, 2,2,2-trifluoro-1,1-dichloroethyl, and the like).

As used herein, the term “amino” means —NH₂. The term “alkylamino” means a nitrogen moiety having one straight or branched unsaturated aliphatic, cyclyl, or heterocyclyl radicals attached to the nitrogen, e.g., —NH(alkyl). The term “dialkylamino” means a nitrogen moiety having at two straight or branched unsaturated aliphatic, cyclyl, or heterocyclyl radicals attached to the nitrogen, e.g., —N(alkyl)(alkyl). The term “alkylamino” includes “alkenylamino,” “alkynylamino,” “cyclylamino,” and “heterocyclylamino.” The term “arylamino” means a nitrogen moiety having at least one aryl radical attached to the nitrogen. For example, —NHaryl, and N(aryl)₂. The term “heteroarylamino” means a nitrogen moiety having at least one heteroaryl radical attached to the nitrogen. For example —NHheteroaryl, and N(heteroaryl)₂. Optionally, two substituents together with the nitrogen can also form a ring. Unless indicated otherwise, the compounds described herein containing amino moieties can include protected derivatives thereof. Suitable protecting groups for amino moieties include acetyl, tertbutoxycarbonyl, benzyloxycarbonyl, and the like. Exemplary alkylamino includes, but is not limited to, NH(C₁-C₁₀alkyl), such as —NHCH₃, NHCH₂CH₃, —NHCH₂CH₂CH₃, and —NHCH(CH₃)₂.

Exemplary dialkylamino includes, but is not limited to, N(C₁-C₁₀alkyl)₂, such as N(CH₃)₂, N(CH₂CH₃)₂, N(CH₂CH₂CH₃)₂, and N(CH(CH₃)₂)₂.

The term “aminoalkyl” means an alkyl, alkenyl, and alkynyl as defined above, except where one or more substituted or unsubstituted nitrogen atoms (—N—) are positioned between carbon atoms of the alkyl, alkenyl, or alkynyl. For example, an (C₂-C₆) aminoalkyl refers to a chain comprising between 2 and 6 carbons and one or more nitrogen atoms positioned between the carbon atoms.

The terms “hydroxyl” and “hydroxyl” mean the radical OH.

The terms “alkoxyl” or “alkoxy” as used herein refers to an alkyl group, as defined above, having an oxygen radical attached thereto, and can be represented by one of —O-alkyl, —O— alkenyl, and —O-alkynyl. Aroxy can be represented by —O-aryl or O-heteroaryl, wherein aryl and heteroaryl are as defined herein. The alkoxy and aroxy groups can be substituted as described above for alkyl. Exemplary alkoxy groups include, but are not limited to O-methyl, O-ethyl, 0-n-propyl, 0-isopropyl, O-n-butyl, 0-isobutyl, O-sec-butyl, 0-tert-butyl, 0-pentyl, O-hexyl, O-cyclopropyl, O-cyclobutyl, O-cyclopentyl, O-cyclohexyl and the like.

As used herein, the term “carbonyl” means the radical C(O)—. It is noted that the carbonyl radical can be further substituted with a variety of substituents to form different carbonyl groups including acids, acid halides, amides, esters, ketones, and the like.

As used herein, the term “oxo” means double bonded oxygen, i.e., ═O.

The term “carboxy” means the radical C(O)O—. It is noted that compounds described herein containing carboxy moieties can include protected derivatives thereof, i.e., where the oxygen is substituted with a protecting group. Suitable protecting groups for carboxy moieties include benzyl, tert-butyl, and the like. As used herein, a carboxy group includes —COOH, i.e., carboxyl group.

The term “ester” refers to a chemical moiety with formula —C(═O)OR, where R is selected from the group consisting of alkyl, cycloalkyl, aryl, heteroaryl and heterocycloalkyl.

The term “cyano” means the radical CN.

The term “nitro” means the radical NO₂.

The term, “heteroatom” refers to an atom that is not a carbon atom. Particular examples of heteroatoms include, but are not limited to nitrogen, oxygen, sulfur and halogens. A “heteroatom moiety” includes a moiety where the atom by which the moiety is attached is not a carbon. Examples of heteroatom moieties include N═, NR^{N}—, N⁺(O⁻)═, —O—, —S— or —S(O)₂—, —OS(O)₂—, and —SS—, wherein R^{N} is H or a further substituent.

The terms “alkylthio” and “thioalkoxy” refer to an alkoxy group, as defined above, where the oxygen atom is replaced with a sulfur. In preferred embodiments, the “alkylthio” moiety is represented by one of —S-alkyl, —S-alkenyl, and —S-alkynyl. Representative alkylthio groups include methylthio, ethylthio, and the like. The term “alkylthio” also encompasses cycloalkyl groups, alkene and cycloalkene groups, and alkyne groups. “Arylthio” refers to aryl or heteroaryl groups.

The term “sulfinyl” means the radical —SO—. It is noted that the sulfinyl radical can be further substituted with a variety of substituents to form different sulfinyl groups including sulfinic acids, sulfinamides, sulfinyl esters, sulfoxides, and the like.

The term “sulfonyl” means the radical —SO₂—. It is noted that the sulfonyl radical can be further substituted with a variety of substituents to form different sulfonyl groups including sulfonic acids (—SO₃H), sulfonamides, sulfonate esters, sulfones, and the like.

The term “thiocarbonyl” means the radical —C(S)—. It is noted that the thiocarbonyl radical can be further substituted with a variety of substituents to form different thiocarbonyl groups including thioacids, thioamides, thioesters, thioketones, and the like.

“Acyl” refers to an aliphatic-CO— group, wherein aliphaticis as previously described. Exemplary acyl groups comprise alkyl of 1 to about 30 carbon atoms. Exemplary acyl groups also include acetyl, propanoyl, 2-methylpropanoyl, butanoyl and palmitoyl.

“Aroyl” means an aryl-CO— group, wherein aryl is as previously described. Exemplary aroyl groups include benzoyl and 1- and 2-naphthoyl.

“Arylthio” refers to an aryl-S— group, wherein the aryl group is as previously described. Exemplary arylthio groups include phenylthio and naphthylthio.

“Aralkyl” refers to an aryl-alkyl- group, wherein aryl and alkyl are as previously described. Exemplary aralkyl groups include benzyl, phenylethyl and naphthylmethyl.

“Aralkyloxy” refers to an aralkyl-O— group, wherein the aralkyl group is as previously described. An exemplary aralkyloxy group is benzyloxy.

“Aralkylthio” refers to an aralkyl-S— group, wherein the aralkyl group is as previously described. An exemplary aralkylthio group is benzylthio.

“Alkoxycarbonyl” refers to an alkyl-O—CO— group. Exemplary alkoxycarbonyl groups include methoxycarbonyl, ethoxycarbonyl, butyloxycarbonyl, and t-butyloxycarbonyl.

“Aryloxycarbonyl” refers to an aryl-O—CO— group. Exemplary aryloxycarbonyl groups include phenoxy- and naphthoxy-carbonyl.

“Aralkoxycarbonyl” refers to an aralkyl-O—CO— group. An exemplary aralkoxycarbonyl group is benzyloxycarbonyl.

“Carbamoyl” refers to an H₂N—CO— group.

“Alkylcarbamoyl” refers to a R′RN—CO— group, wherein one of R and R′ is hydrogen and the other of R and R′ is alkyl as previously described.

“Dialkylcarbamoyl” refers to R′RN—CO— group, wherein each of R and R′ is independently alkyl as previously described.

“Acyloxy” refers to an acyl-O— group, wherein acyl is as previously described. “Acylamino” refers to an acyl-NH— group, wherein acyl is as previously described. “Aroylamino” refers to an aroyl-NH— group, wherein aroyl is as previously described.

The term “optionally substituted” means that the specified group or moiety is unsubstituted or is substituted with one or more (typically 1, 2, 3, 4, 5 or 6 substituents) independently selected from the group of substituents listed below in the definition for “substituents” or otherwise specified. The term “substituents” refers to a group “substituted” on a substituted group at any atom of the substituted group. Suitable substituents include, without limitation, halogen, hydroxyl, caboxy, oxo, nitro, haloalkyl, alkyl, alkenyl, alkynyl, alkaryl, aryl, heteroaryl, cyclyl, heterocyclyl, aralkyl, alkoxy, aryloxy, amino, acylamino, alkylcarbanoyl, arylcarbanoyl, aminoalkyl, alkoxycarbonyl, carboxy, hydroxylalkyl, alkanesulfonyl, arenesulfonyl, alkanesulfonamido, arenesulfonamido, aralkylsulfonamido, alkylcarbonyl, acyloxy, cyano or ureido. In some cases, two substituents, together with the carbons to which they are attached to can form a ring.

For example, any alkyl, alkenyl, cycloalkyl, heterocyclyl, heteroaryl or aryl is optionally substituted with 1, 2, 3, 4 or 5 groups selected from OH, CN, —SC(O)Ph, oxo (═O), SH, SO₂NH₂, SO₂(C₁-C₄)alkyl, SO₂NH(C₁-C₄)alkyl, halogen, carbonyl, thiol, cyano, NH₂, NH(C₁-C₄)alkyl, N[(C₁-C₄)alkyl]₂, C(O)NH₂, COOH, COOMe, acetyl, (C₁-C₈)alkyl, O(C₁-C₈)alkyl, O(C₁-C₈)haloalkyl, (C₂-C₈)alkenyl, (C₂-C₈)alkynyl, haloalkyl, thioalkyl, cyanomethylene, alkylaminyl, aryl, heteroaryl, substituted aryl, NH₂—C(O)-alkylene, NH(Me)-C(O)-alkylene, CH₂—C(O)-alkyl, C(O)-alkyl, alkylcarbonylaminyl, CH₂—[CH(OH)]ₘ—(CH₂)ₚ—OH, CH₂—[CH(OH)]ₘ—(CH₂)ₚ—NH₂ or CH₂-aryl-alkoxy; “m” and “p” are independently 1, 2, 3, 4, 5 or 6.

In some embodiments, an optionally substituted group is substituted with 1 substituent. In some other embodiments, an optionally substituted group is substituted with 2 independently selected substituents, which can be same or different. In some other embodiments, an optionally substituted group is substituted with 3 independently selected substituents, which can be same, different or any combination of same and different. In still some other embodiments, an optionally substituted group is substituted with 4 independently selected substituents, which can be same, different or any combination of same and different. In yet some other embodiments, an optionally substituted group is substituted with 5 independently selected substituents, which can be same, different or any combination of same and different.

An “isocyanato” group refers to a NCO group.

A “thiocyanato” group refers to a CNS group.

An “isothiocyanato” group refers to a NCS group.

“Alkoyloxy” refers to a RC(═O)O— group.

“Alkoyl” refers to a RC(═O)— group.

As used herein, the terms “dsRNA”, “siRNA”, and “iRNA agent” are used interchangeably to refer to agents that can mediate silencing of a target RNA, e.g., mRNA, e.g., a transcript of a gene that encodes a protein. For convenience, such mRNA is also referred to herein as mRNA to be silenced. Such a gene is also referred to as a target gene. In general, the RNA to be silenced is an endogenous gene, exogenous gene or a pathogen gene. In addition, RNAs other than mRNA, e.g., tRNAs, and viral RNAs, can also be targeted.

As used herein, the phrase “mediates RNAi” refers to the ability to silence, in a sequence specific manner, a target gene, e.g., mRNA. While not wishing to be bound by theory, it is believed that silencing uses the RNAi machinery or process and a guide RNA, e.g., antisense strand of a dsRNA, where the antisense strand is 21 to 23 nucleotides in length.

As used herein, “specifically hybridizable” and “complementary” are terms which are used to indicate a sufficient degree of complementarity such that stable and specific binding occurs between a compound of the invention and a target RNA molecule. Specific binding requires a sufficient degree of complementarity to avoid non-specific binding of the oligomeric compound to non-target sequences under conditions in which specific binding is desired, i.e., under physiological conditions in the case of assays or therapeutic treatment, or in the case of in vitro assays, under conditions in which the assays are performed. The non-target sequences typically differ by at least 5 nucleotides.

In some embodiments, a dsRNA molecule of the invention is “sufficiently complementary” to a target RNA, e.g., a target mRNA, such that the dsRNA molecule silences production of protein encoded by the target mRNA. In another embodiment, the dsRNA molecule of the invention is “exactly complementary” to a target RNA, e.g., the target RNA and the dsRNA duplex agent anneal, for example to form a hybrid made exclusively of Watson-Crick base pairs in the region of exact complementarity. A “sufficiently complementary” target RNA can include an internal region (e.g., of at least 10 nucleotides) that is exactly complementary to a target RNA. Moreover, in some embodiments, the dsRNA molecule of the invention specifically discriminates a single-nucleotide difference. In this case, the dsRNA molecule only mediates RNAi if exact complementary is found in the region (e.g., within 7 nucleotides of) the single-nucleotide difference.

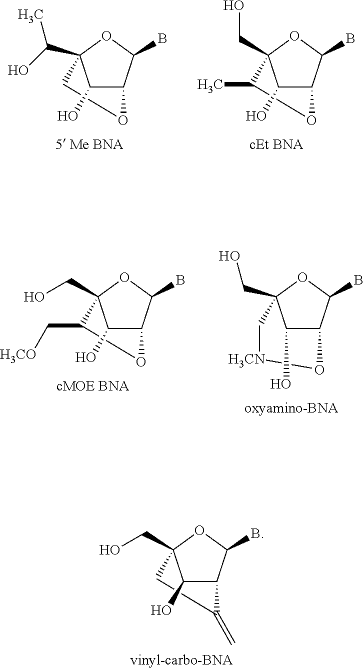

The term ‘BNA’ refers to bridged nucleic acid, and is often referred as constrained or inaccessible RNA. BNA can contain a 5-, 6-membered, or even a 7-membered bridged structure with a “fixed” C3′-endo sugar puckering. The bridge is typically incorporated at the 2′-, 4′-position of the ribose to afford a 2′, 4′-BNA nucleotide (e.g., LNA, or ENA). Examples of BNA nucleotides include the following nucleosides:

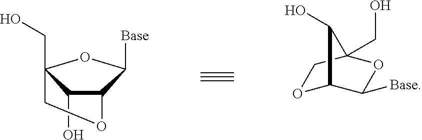

The term ‘ENA’ refers to ethylene-bridged nucleic acid, and is often referred as constrained or inaccessible RNA.

The “cleavage site” herein means the backbone linkage in the target gene or the sense strand that is cleaved by the RISC mechanism by utilizing the iRNA agent. And the target cleavage site region comprises at least one or at least two nucleotides on both side of the cleavage site. For the sense strand, the cleavage site is the backbone linkage in the sense strand that would get cleaved if the sense strand itself was the target to be cleaved by the RNAi mechanism. The cleavage site can be determined using methods known in the art, for example the 5′-RACE assay as detailed in Soutschek et al., Nature (2004) 432, 173-178, which is incorporated by reference in its entirety. As is well understood in the art, the cleavage site region for a conical double stranded RNAi agent comprising two 21-nucleotides long strands (wherein the strands form a double stranded region of 19 consecutive base pairs having 2-nucleotide single stranded overhangs at the 3′-ends), the cleavage site region corresponds to positions 9-12 from the 5′-end of the sense strand.

The terms “decrease”, “reduced”, “reduction”, or “inhibit” are all used herein to mean a decrease by a statistically significant amount. In some embodiments, “reduce,” “reduction” or “decrease” or “inhibit” typically means a decrease by at least 10% as compared to a reference level (e.g. the absence of a given treatment) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” does not encompass a complete inhibition or reduction as compared to a reference level. “Complete inhibition” is a 100% inhibition as compared to a reference level. A decrease can be preferably down to a level accepted as within the range of normal for an individual without a given disorder.

As used herein, a “terminal region” of a strand refers to positions 1-4, e.g., positions 1, 2, 3, and 4, counting from the nearest end of the strand. For example, a 5′-terminal region refers to positions 1-4, e.g., positions 1, 2, 3 and 4 counting from the 5′-end of the strand. Similarly, a 3′-terminal region refers to positions 1-4, e.g., positions 1, 2, 3 and 4 counting from the 3′-end of the strand.

For example, a 5′-terminal region for the antisense strand is positions 1, 2, 3 and 4 counting from the 5′-end of the antisense strand. A preferred 5′-terminal region for the antisense strand is positions 1, 2 and 3 counting from the 5′-end of the antisense strand. A 3′-terminal region for the antisense strand can be positions 1, 2, 3, and 4 counting from the 3′-end of the strand. A preferred 3′-terminal region for the antisense strand is positions 1, 2 and 3 counting from the 3′-end of the antisense strand.

Similarly, a 5′-terminal region for the sense strand is positions 1, 2, 3 and 4 counting from the 5′-end of the sense strand. A preferred 5′-terminal region for the sense strand is positions 1, 2 and 3 counting from the 5′-end of the sense strand. A 3′-terminal region for the sense strand can be positions 1, 2, 3, and 4 counting from the 3′-end of the strand. A preferred 3′-terminal region for the sense strand is positions 1, 2 and 3 counting from the 3′-end of the sense strand.

As used herein, a “central region” of a strand refers to positions 5-17, e.g., positions 6-16, positions 6-15, positions 6-14, positions 6-13, positions 6-12, positions 7-15, positions 7-14, positions 7-13, positions, 7-12, positions 8-16, positions 8-15, positions 8-14, positions 8-13, positions 8-12, positions 9-16, positions 9-15, positions 9-14, positions 9-13, positions 9-12, positions 10-16, positions 10-15, positions 10-14, positions 10-13 or positions 10-12, counting from the 5′-end of the strand. For example, the central region of a strand means positions 5, 6, 7, 8, 9, 10, 11, 12, 13, 14, 15, 16 or 17 of the strand. A preferred central region for the sense strand is positions 6, 7, 8, 9, 10, 11, 12, 13, and 14, counting from the 5′-end of the sense strand. A more preferred central region for the sense strand is positions 7, 8, 9, 10, 11, 12 and 13, counting from the 5′-end of the sense strand. A preferred central region for the antisense strand is positions 9, 10, 11, 12, 13, 14, 15 16 and 17, counting from 5′-end of the antisense strand. A more preferred central region for the antisense strand is positions 10, 11, 12, 13, 14, 15 and 16, counting from 5′-end of the antisense strand.

As will be apparent to those of skill in the art upon reading this disclosure, each of the individual aspects described and illustrated herein has discrete components and features which can be readily separated from or combined with the features of any of the other several aspects without departing from the scope or spirit of the present invention. Any recited method can be carried out in the order of events recited or in any other order which is logically possible.

### EXAMPLES

The invention is further illustrated by the following examples, which should not be construed as further limiting. The contents of all references, pending patent applications and published patents, cited throughout this application are hereby expressly incorporated by reference.

### Example 1: NMA Conjugates

Conjugation of siRNAs with lipophilic compounds has been shown to improve cellular uptake and biodistribution through in vivo studies with cholesterol conjugated siRNAs. The 2′-O-[2-(ethoxy)-2-oxoethyl]ester linkage has not been explored for conjugation of lipophilic and other conjugates like GalNAc. In this study, inventors made modifications to this linker with lipophilic amines of various carbon lengths. The inventors evaluated both primary amines and secondary amines.

This work was done to help the delivery of siRNA molecules into the cell and to improve the biodistribution within the body to different types of cells. Biodistribution is very important with these drugs because the more types of tissues and cells to which we can deliver, more types of diseases and disorders we will be able to treat and cure.

The inventors used two different approaches. In the first approach, they synthesized oligonucleotides with the starting ester containing phosphoramidites, and carried out post-synthesis conjugation reactions on the solid supported oligonucleotides. This approach allowed us conjugation with a variety of amines at a much quicker pace. In the second approach, the inventors synthesized starting nucleosides (e.g., U, C and A) with the ester linkage to make amide linkers with long alkyl chains. Then they used these nucleoside phosphoramidites to make various oligonucleotides.

Small interfering RNA (siRNA) molecules are a unique class of double stranded non-coding RNA molecules that operate within the RNA interference (RNAi) pathway. These molecules can be used to interfere with the expression of particular genes, usually those that when expressed will cause diseases within the body. A single strand of the siRNA molecule will bind with its complementary mRNA strand within the cell and causes the mRNA to cleave. The cell then recognizes the cut mRNA as irregular or abnormal and degrades it into its individual nucleotides, which can then be recycled for further translation. In turn, the proteins that specific mRNA strand codes for are not created, and the corresponding gene is not expressed.

Nucleic acids, based on their inherent chemical make-up, are hydrophilic molecules. Due to this, they have poor membrane transport and bio-distribution within the body. Normally, within the translation and transcription world of RNA, this is an acceptable limitation. However, in the siRNA world, the hydrophilicity of the molecules has proven to be an obstacle when attempting to deliver the drugs. Lipophilic compounds, which are hydrophobic, can help improve these properties of nucleic acid, cellular upkeep, and siRNA pharmacology. Conjugating siRNA molecules with lipophilic compounds will help ensure the interaction between the siRNA molecules and the cellular membrane. It has been proven that lipophilic compounds can bind and form complexes with high density lipids (HDLs), low density lipids (LDLs), and serum albumin. These complexes can then bind to the appropriate receptors on the surface of the membrane, which recognize specific components of the complex, and subsequently go through endocytosis into the cell.

The NMA (N-methyl acetamide) chemical modification at the 2′-O-position of RNA monomers has been proven to improve its metabolic stability, cellular adsorption, and protein binding properties. It has been postulated that nucleosides that contain the NMA modification will retain the “gauche” conformation, which occurs when groups around an atom are separated by a torsion angle of 600 as opposed to the more common anti-configuration of 180°. This also leads to the C3-endo sugar pucker characteristic of the RNA molecule to be retained. Oligonucleotides with the NMA modification exhibit strong binding affinity to complementary RNA strands, and more importantly not to DNA strands. It also stabilizes the 3′-exonuclease stability of oligonucleotides, giving a t_{1/2} of greater than 24 hours, which helps prevent the RNA from cleaving in the cells. The NMA modification was made through a methyl ester group on the 2′ carbon of the sugar.

The NMA modification is very similar to the methoxyethyl (MOE) modification, and can be made on the same 2′ carbon. The MOE modification secures the C3 of the sugar in the endo conformation, which helps pre-organize the siRNA oligonucleotide preferable for mRNA target binding. In addition, MOE modified strands are shown to have water structures formed around them. NMA modified strands similarly form stable hydrated structures that help with the mRNA binding affinity of the siRNA strands.

A study was done comparing the in vivo and in vitro activity of 2′-O-NMA and 2′-O-MOE modified antisense oligonucleotide strands. Single strand antisense oligonucleotides (ASOs) were modified with NMA and MOE modifications flanked at the 3′ and 5′ ends. They were oriented as “gapmers” with five modified bases on each end and ten unmodified bases in the middle all linked by a uniform P═S backbone. The NMA modified gapmers showed increased affinity to complementary RNA and reduced expression of PTEN mRNA, which leads to a tumor suppressor gene, in vitro and in vivo when compared to its MOE modified counterparts.

Another study was done on the addition of a vinyl phosphonate (VP) linker to NMA modified oligonucleotides. The VP linker was incorporated on the 5′ end of the antisense strand of the modified siRNA. Tests were run on four different strands: a base strand (no modification), an NMA modified strand (2_{NMA}), a VP linked strand (2E-VP), and an NMA modified strand with a VP linker (2E-VP_{NMA}). These conjugates, all targeting ApoB, were monitored in mice at two different doses, 10 mg/kg and 3 mg/kg, and LDL levels were measured after one week. The 2_{NMA} modified strand did not show any considerable activity at either dose. The 2E-VP strand did show considerable activity at both doses. This proves that 5′-VP linker and the 2′-NMA modification together has benefits and can improve the oligonucleotides activity.

The NMA chemistry, and all its previous research, has opened the door for many other modifications through a similar conjugation method (eg. GalNAc, lipids, and other targeting ligands), which can help further improve stability and transport of RNA, and siRNA, molecules. A specific modification of interest, which will be further discussed in this report, is the addition of lipophilic amines with an amide linkage of a long hydrocarbon chain. This addition can act as “grease” for siRNA molecules to help with their delivery into the cell. This modification can be made through an ethyl ester group on the 2′-carbon of the sugar through a complementary approach for conjugation chemistry. Commonly, oligonucleotide conjugation chemistry is done through a nucleophilic site with an electrophile externally brought in to carry out the reaction. The available starting material allowed the inventors to reverse this chemistry given an ethyl ester group at the 2′ carbon of the sugar. In this case, the electrophile (carbonyl carbon of the ester) acts as the reaction site, and a reactive nucleophile (amino group) of the ligand is brought into the site and allowed to react. This allowed the inventors to expand the NMA modification and extend the hydrocarbon chain much past a small methyl group.

### Results and Discussion

The work was carried out via two approaches: post synthetic conjugation and pre synthetic conjugation. The former was done at the oligonucleotide stage in the solid support, while the latter was done at the nucleoside stage. There were pros and cons to each approach, and they will be discussed in detail later in this report.

The first step of the post synthetic conjugation approach was to create phosphoramidites from the given starting material (2′-ester nucleosides). The bases used were uracil, 5-methyl cytosine, and adenine.

Compounds 1, 3, and 5 were synthesized by a previously published procedure where the methyl ester analogues were demonstrated¹⁻³. Additionally, with the known phosphoramidite procedure, compounds 2, 4, and 6 were synthesized.^{1,2}

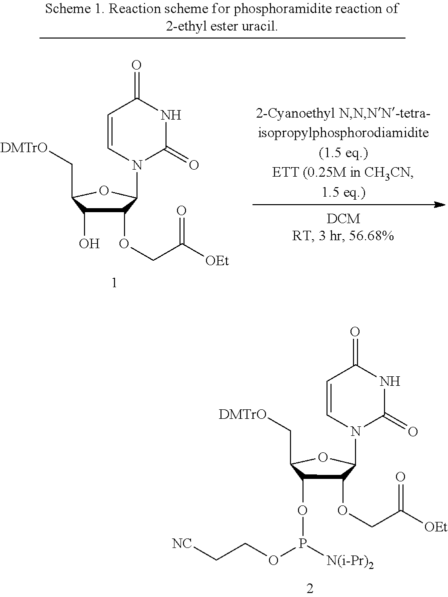

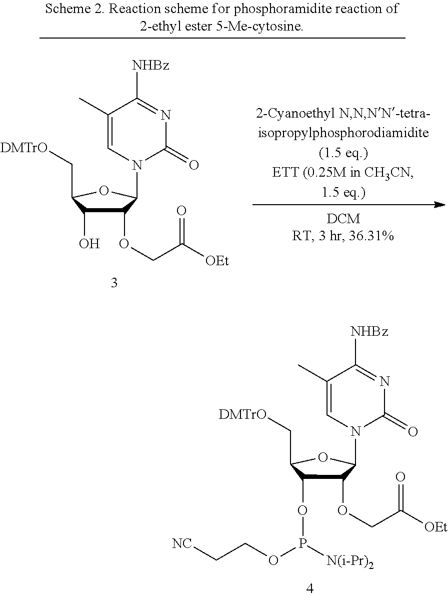

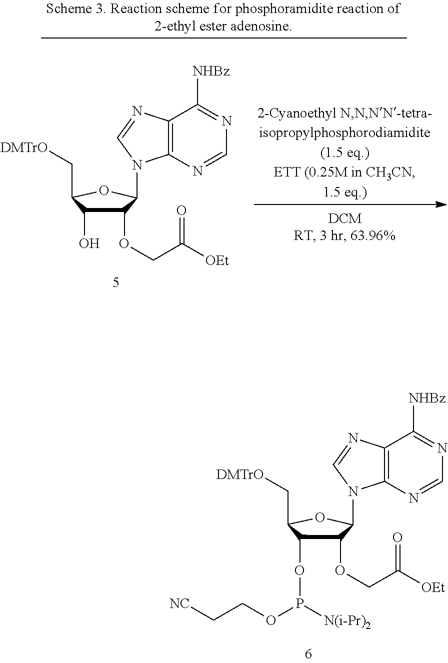

The next step was to synthesize oligonucleotide strands with one of the phosphoramidites being one shown in Table 1. These oligonucleotides were synthesized in solid support so that conjugation reactions could be done from there. The oligonucleotides synthesized were 20 nucleosides long consisting of 19 thymine nucleosides (dT) and one modified phosphoramidite from the above table at the tenth position. The amines used for conjugation were hexadecamine, oleyl amine, tetradecanamine, and octadecanamine. Table 2 below shows some exemplary non-conjugated and conjugated oligonucleodites that were synthesized.

TABLE 1
Non-Cojugated Phosphoramidite

| Compound Number | Name | Structure |
|---|---|---|
| 2 | 2′ester U | 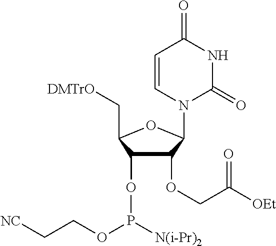 |
| 4 | 2′ester 5-Me C | 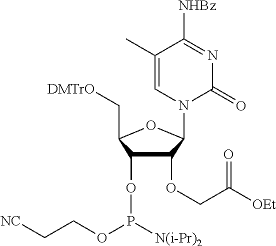 |
| 6 | 2′ester A | 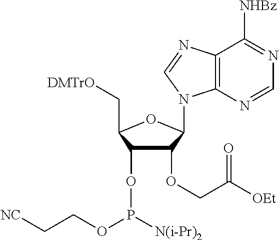 |

TABLE 2
Oligonucleotides Pre- and Post-Conjugation Reactions with Various Amines

| | | | | SEQ | |
|---|---|---|---|---|---|
| Serial | | Mass | Mass | ID | |
| Number | Sequence | exp | obs | NO: | Structure of the conjugates |
| 1 | 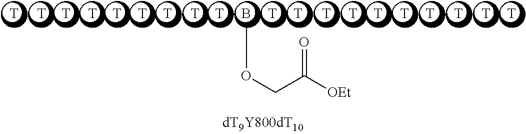 | SM | N/A | 1 | 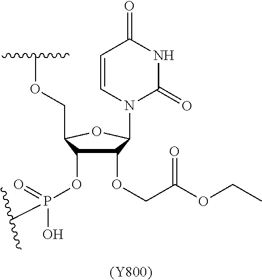 |
| 2 | 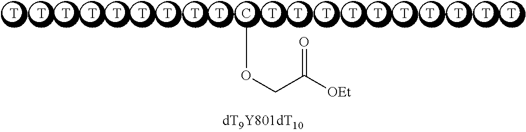 | SM | N/A | 2 | 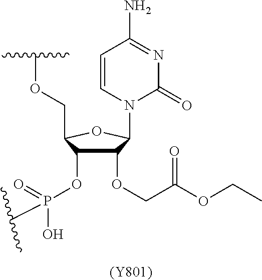 |
| 3 | 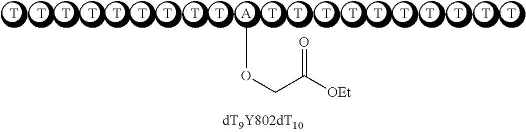 | SM | N/A | 3 | 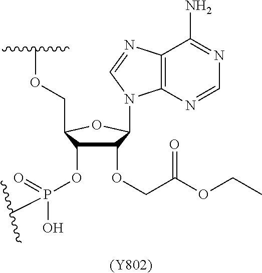 |
| 4 | 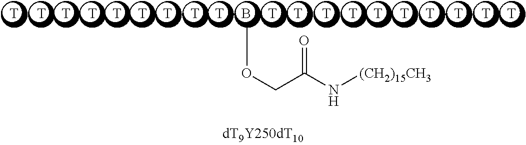 | 6304 | 6303 | 4 | 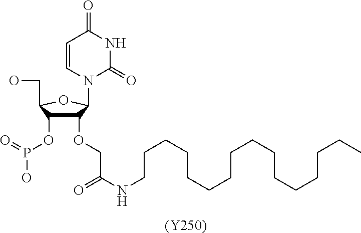 |
| 5 | 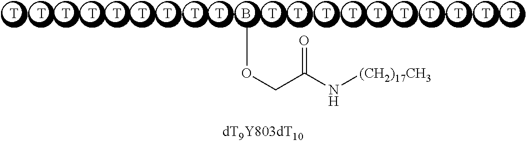 | 6332 | 6317 | 5 | 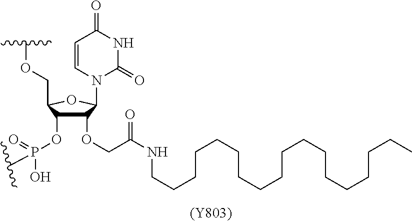 |
| 6 | 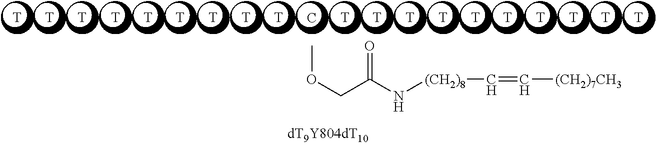 | 6343 | 6343 | 6 | 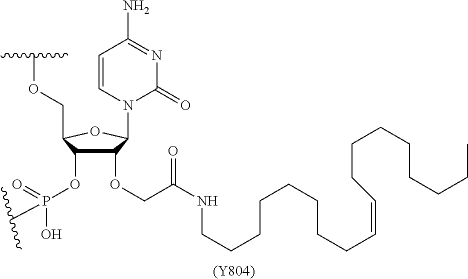 |
| 7 | 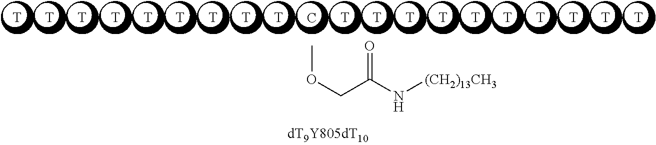 | 6289 | 6289 | 7 | 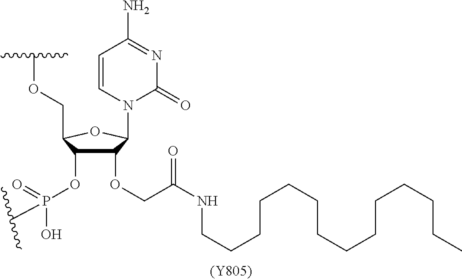 |

The first step of the pre synthetic conjugation approach was to conjugate individual nucleosides, uracil, 5-methyl cytosine, and adenine, with a given amine. The first amine used was hexadecanamine. After conjugation reactions with all the bases were complete, the same phosphoramidite reaction previously mentioned was performed. The scheme below displays the synthetic procedure for the conjugation of 2′-ethyl ester uracil with hexadecanamine and the subsequent phosphoramidite reaction.

Compound 7 was synthesized via the proposed synthesis laid out in the experimental portion of this report. The yield was 77% and the compound was characterized with mass spectroscopy and nuclear magnetic resonance. Compound 7 was then used to make compound 8, which was synthesized via the known phosphoramidite procedure⁴. This reaction had a 55% yield.

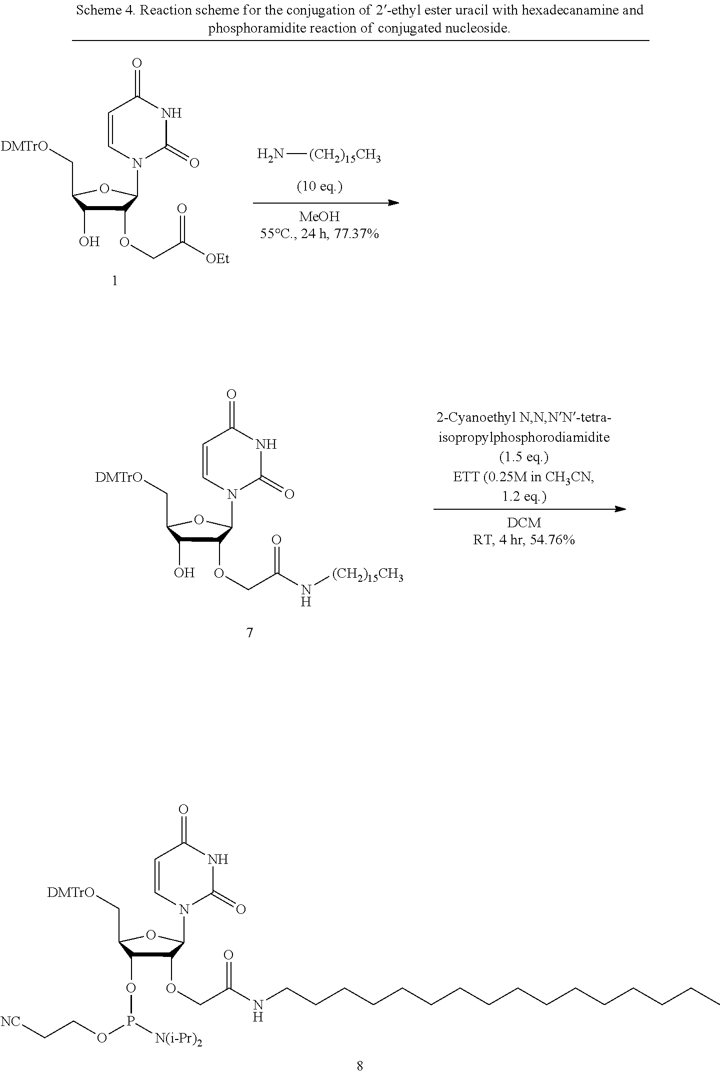

Compounds 9 and 12 were prepared following the similar procedure used for compound 7. While compounds 11 and 14 were again prepared with the known phosphoramidite procedure. Table 3 below shows the conjugated phosphoramidites synthesized:

TABLE 3
Conjugated Phosphoramidites

| Compound | | |
|---|---|---|
| Number | Name | Structure |
| 8 | C-16 conjugated U | 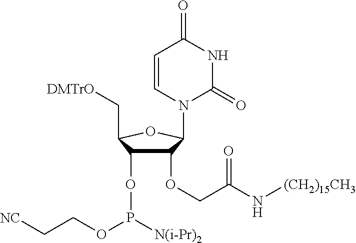 |
| 11 | C-16 conjugated 5-Me C | 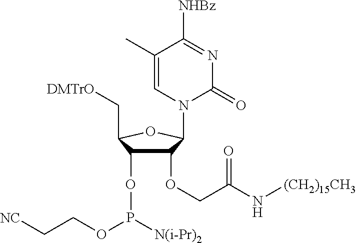 |
| 14 | C-16 conjugated A | 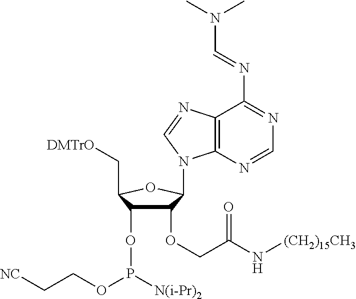 |

The 5-methyl cytosine nucleoside was also conjugated with a secondary amine, dihexylamine. This procedure was a little different than the primary amine conjugations and required the additional reagent pyridine. Additionally, the benzoyl protective group on the amine group of the cytosine base was not removed during this reaction. Whereas with the primary amine conjugation, it was removed for the 5-methyl cytosine and adenine base nucleosides. This proves that under the conditions laid out in the reaction scheme below, nucleoside conjugation with secondary amines is possible. Therefore, further conjugation reactions with other secondary amines, following the same procedure, can be completed. However, the conjugated nucleoside has not been converted into a phosphoramidite. Compound 15 was prepared via a similar procedure to that of compound 7 with a yield of 67%.

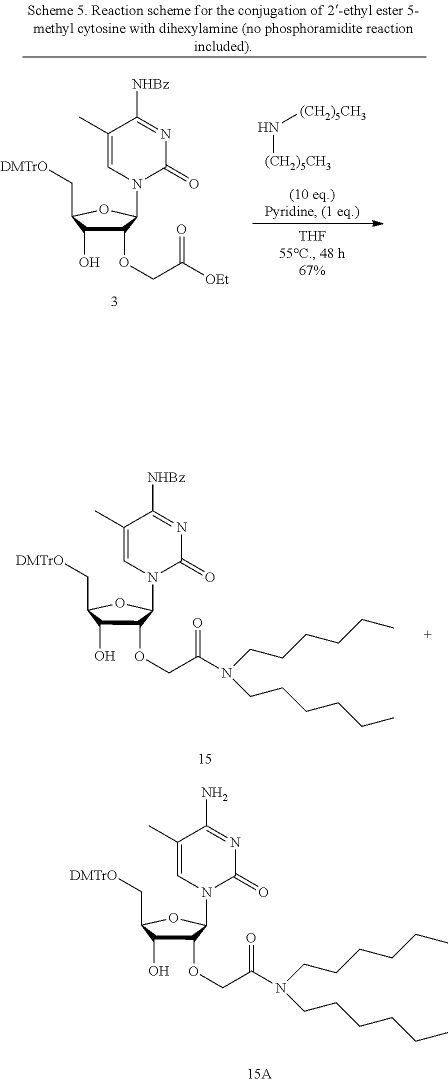

Both approaches were successful in chemically creating oligonucleotide strands with a modified base containing an amide linkage and a long hydrocarbon chain. The advantage to the post synthetic approach is that with one oligonucleotide strand in the solid support, multiple conjugation reactions can be made at the same time. This will yield many different modifications of the strand, and will determine what modification might work best. The disadvantage to this approach is that it does not always create the correct modified strand. The advantage to the pre synthetic approach is that the correct modified nucleoside can be made with 100% accuracy and then can be used to produce one oligonucleotide strand. The disadvantage to this approach is that only one modification can be made at a time and the process is slower.

### Conclusion

Multiple molecules were synthesized during this project and can be seen in the tables and figures above. These molecules have not yet been tested for their ability to improve biochemical/biophysical properties of the drugs, but that will be further investigated in the future. The most effective way to move forward would be to test multiple conjugations using the post synthetic approach and find out which positively impacts the necessary properties the most. Then nucleosides can be conjugated with that specific modification and mass produced so that they can be implemented into any given siRNA strand. One modification that is of interest is the GalNAc amine, which has already shown elements of improving siRNA drugs. Both mono and tri GalNAc additions can be further investigated. Additionally, the polymerase activity of these nucleoside conjugates can be investigated. The use of this novel linker can be incorporated into various siRNAs within multiple positions of sense or antisense strands. This project has opened a gateway to more possibilities in improving siRNA drugs and their effectiveness as a therapy.

### Methods and Materials

Post Synthetic Conjugation Procedure: Three dT-20mers sequences were synthesized with the ester linked phosporamidites substituted at position ten of each sequence.

To a solution of solid support (2 mg, Sr. No. 1 and 2) and 25 μL of water, a 10-50% solution of various amines in 25 μL of Ethanol was added. The mixture was heated at 90° C. for 60 minutes. It was then treated with 50 μL of a 40% aqueous methylamine solution and heated at 60° C. for 12 minutes. The mixture was next worked up with 200 μL of CH₂Cl₂ and 200 μL of water. The top aqueous layer was extracted and examined via mass spectrometry.

Oligonucleotides 4, 5, and 6 all displayed a 100% mass spectrometry correlation between the observed mass and expected mass. Oligonucleotide 7 only displayed an 80% correlation.

### Synthetic Procedures

General conditions: TLC was performed on Merck silica gel 60 plates coated with F254. Compounds were visualized under UV light (254 nm) or after spraying with the p-anisaldehyde staining solution followed by heating. Flash column chromatography was performed using a Teledyne ISCO Combi Flash system with pre-packed RediSep Teledyne ISCO silica gel cartridges. All moisture-sensitive reactions were carried out under anhydrous conditions using dry glassware, anhydrous solvents, and argon atmosphere. All commercially available reagents and solvents were purchased from Sigma-Aldrich unless otherwise stated and were used as received. ESI-MS spectra were recorded on a Waters QT of Premier instrument using the direct flow injection mode. ¹H NMR spectra were recorded at 500 and 600 MHz. ¹³C NMR spectra were recorded at 126 and 151 MHz. ³¹P NMR spectra were recorded at 202 and 243 MHz. Chemical shifts are given in ppm referenced to the solvent residual peak (DMSO-d₆—1H: δ at 2.50 ppm and ¹³C δ at 39.5 ppm; CDCl₃ —1H: δ at 7.26 ppm and ¹³C δ at 77.16 ppm). Coupling constants are given in Hertz. Signal splitting patterns are described as singlet (s), doublet (d), triplet (t), septet (sept), broad signal (brs), or multiplet (m).

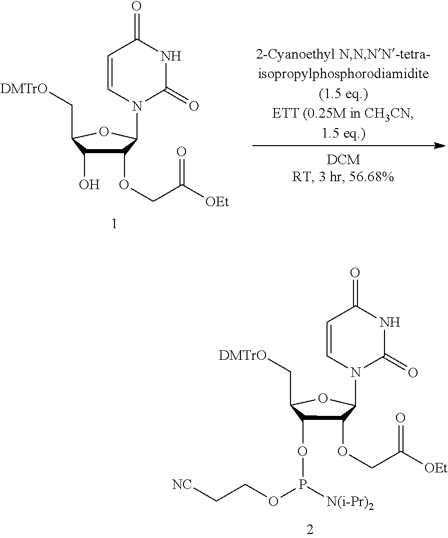

Compound 2 (ELN0069-026): To a solution of 1(6.5 g, 10.27 mmol) and 5-ethylthio-1H-tetrazole (0.25 M in CH₃CN, 61.64 mL, 15.41 mmol) in CH₂Cl₂ (20 mL) were added dropwise 2-cyanoethyl N,N,N′,N′-tetraisopropylphosphorodiamidite (4.89 mL, 15.41 mmol) at 0° C. The mixture was stirred at rt for 3 h. The reaction mixture was quenched with saturated NaHCO₃(aq.) and diluted with ethyl acetate. The organic layer was washed with saturated NaHCO₃(aq.), brine, dried (Na₂SO₄) and concentrated under vacuum. The crude residue was purification by column chromatography on silica gel (0-50% ethyl acetate in hexanes) to obtain the desired compound 2 as a white form (4.85 g, 57%). ¹H NMR (400 MHz, CD₃CN) δ 8.98 (s, 1H), 7.77-7.60 (m, 1H), 7.48-7.38 (m, 2H), 7.37-7.20 (m, 7H), 6.93-6.83 (m, 4H), 5.94 (dd, J=9.1, 4.1 Hz, 1H), 5.28 (dd, J=8.1, 3.4 Hz, 1H), 4.53-4.42 (m, 1H), 4.39-4.10 (m, 6H), 3.92-3.54 (m, 9H), 3.47-3.29 (m, 2H), 2.67 (ddd, J=6.5, 5.4, 2.9 Hz, 1H), 2.52 (q, J=6.1 Hz, 1H), 1.27-1.10 (m, 13H), 1.04 (d, J=6.8 Hz, 4H) ppm. ¹³C NMR (151 MHz, CD₃CN) δ 170.42, 170.25, 163.47, 163.46, 159.38, 159.37, 159.35, 150.99, 150.95, 145.34, 145.29, 140.99, 140.75, 136.02, 135.99, 135.94, 135.86, 130.79, 130.73, 130.72, 128.65, 128.62, 128.52, 127.58, 119.22, 119.01, 113.71, 102.25, 102.20, 88.35, 87.86, 87.31, 87.26, 83.39, 83.37, 83.10, 83.06, 82.05, 82.03, 81.51, 81.48, 70.98, 70.88, 70.79, 68.14, 68.13, 67.85, 67.84, 62.83, 62.47, 61.25, 59.31, 59.19, 58.84, 58.70, 55.52, 55.49, 43.66, 43.62, 43.58, 43.54, 24.61, 24.58, 24.55, 24.53, 24.49, 24.44, 24.39, 20.62, 20.60, 20.58, 20.56, 14.10, 14.08. ³¹P NMR (162 MHz, CD₃CN) δ 150.01, 149.55 ppm. HRMS calc. for C₄₃H₅₄N₄O₁₁P [M+H]⁺ 833.3527, found 833.3535.

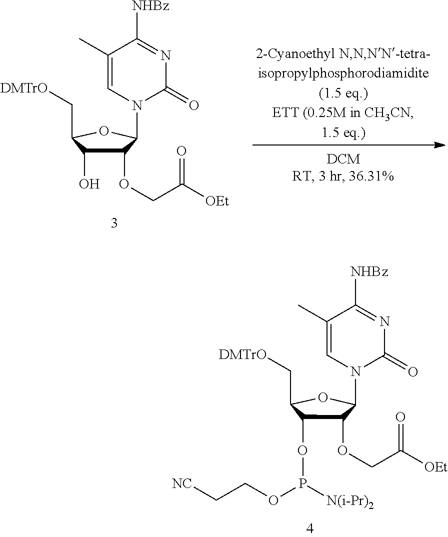

Compound 4 (MN-0069-027): To a solution of 3 (5 g, 6.67 mmol) and 5-ethylthio-1H-tetrazole (0.25 M in CH₃CN, 40 mL, 10.00 mmol) in CH₂Cl₂ (20 mL) were added dropwise 2-cyanoethyl-N,N,N′,N′-tetraisopropylphosphorodiamidite (3.18 mL, 10.00 mmol) at 0° C. The mixture was stirred at rt for 3 h. The reaction mixture was quenched with saturated NaHCO₃(aq.) and diluted with ethyl acetate. The organic layer was washed with saturated NaHCO₃(aq.), brine, dried (Na₂SO₄) and concentrated under vacuum. The crude residue was purification by column chromatography on silica gel (0-60% ethyl acetate in hexanes) to obtain the desired compound 4 as a white form (2.30 g, 36%). ¹H NMR (400 MHz, CD₃CN) δ 13.24 (s, 1H), 8.27 (d, J=7.6 Hz, 2H), 7.87-7.71 (m, 1H), 7.62-7.53 (m, 1H), 7.40-7.21 (m, 7H), 6.89 (ddd, J=9.0, 6.3, 1.8 Hz, 4H), 6.04 (dd, J=7.6, 4.5 Hz, 1H), 4.56 (dt, J=10.8, 5.1 Hz, 1H), 4.45-4.25 (m, 3H), 4.25-4.11 (m, 3H), 3.92-3.67 (m, 8H), 3.67-3.52 (m, 2H), 3.51-3.28 (m, 2H), 2.76-2.57 (m, 1H), 2.50 (t, J=5.9 Hz, 1H), 1.60 (dd, J=9.1, 1.1 Hz, 3H), 1.22-1.13 (m, 10H), 1.03 (d, J=6.8 Hz, 4H) ppm. ¹³C NMR (151 MHz, CD₃CN) δ 179.98, 170.43, 170.30, 160.98, 159.44, 159.42, 148.55, 145.31, 145.28, 138.55, 137.75, 136.05, 136.02, 135.99, 135.93, 133.08, 130.79, 130.76, 130.74, 130.16, 128.80, 128.70, 128.64, 128.60, 128.59, 127.69, 127.67, 119.25, 119.01, 113.76, 113.75, 111.90, 88.54, 88.15, 87.33, 87.31, 83.85, 83.83, 83.56, 83.52, 82.08, 82.06, 81.46, 81.43, 71.13, 71.04, 70.94, 68.15, 68.13, 67.84, 67.82, 63.06, 62.73, 61.26, 59.33, 59.21, 58.78, 58.64, 55.53, 55.51, 43.68, 43.62, 43.60, 43.54, 24.60, 24.58, 24.55, 24.53, 24.48, 24.45, 24.44, 24.40, 20.65, 20.61, 20.56, 14.10, 14.08, 12.76, 12.69. ³¹P NMR (162 MHz, CD₃CN) δ 150.03, 149.67 ppm. HRMS calc. for C₅₁H₆₁N₅O₁₁P [M+H]⁺ 950.4105, found 950.4113.

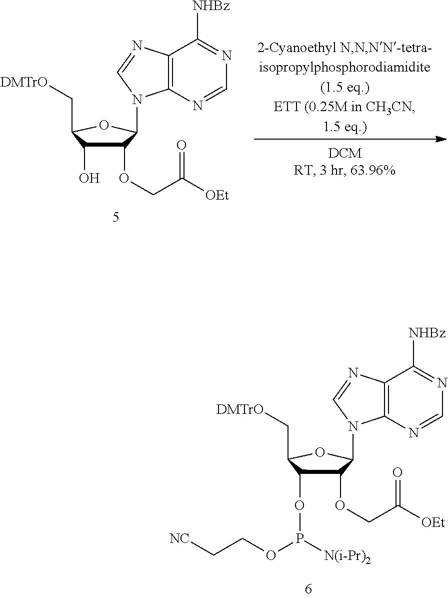

Compound 6 (MN-0069-028): To a solution of 5 (5 g, 6.58 mmol) and 5-ethylthio-1H-tetrazole (0.25 M in CH₃CN, 39.5 mL, 9.87 mmol) in CH₂Cl₂ (20 mL) were added dropwise 2-cyanoethyl-N,N,N′,N′-tetraisopropylphosphorodiamidite (3.13 mL, 9.87 mmol) at 0° C. The mixture was stirred at rt for 3 h. The reaction mixture was quenched with saturated NaHCO₃(aq.) and diluted with ethyl acetate. The organic layer was washed with saturated NaHCO₃(aq.), brine, dried (Na₂SO₄) and concentrated under vacuum. The crude residue was purification by column chromatography on silica gel (0-50% ethyl acetate in hexanes) to obtain the desired compound 6 as a white form (4.04 g, 64%). ¹H NMR (400 MHz, CD₃CN) δ 9.30 (s, 1H), 8.59 (d, J=3.3 Hz, 1H), 8.33 (d, J=7.2 Hz, 1H), 8.00 (d, J=7.7 Hz, 2H), 7.69-7.60 (m, 1H), 7.48-7.34 (m, 2H), 7.32-7.12 (m, 7H), 6.85-6.71 (m, 4H), 6.24 (dd, J=13.4, 4.7 Hz, 1H), 4.98 (dt, J=14.4, 4.8 Hz, 1H), 4.91-4.75 (m, 1H), 4.45-4.15 (m, 3H), 4.08-4.00 (m, 1H), 3.98-3.78 (m, 1H), 3.77-3.53 (m, 9H), 3.43 (ddd, J=17.9, 10.8, 3.3 Hz, 1H), 3.37-3.21 (m, 1H), 2.68 (dt, J=6.4, 5.1 Hz, 1H), 2.51 (t, J=6.0 Hz, 1H), 1.18 (ddd, J=6.8, 5.1, 3.4 Hz, 10H), 1.14-1.06 (m, 7H) ppm. ¹³C NMR (151 MHz, CD₃CN) δ 170.56, 170.31, 159.23, 159.21, 159.19, 152.47, 150.56, 145.55, 145.54, 143.67, 143.40, 136.34, 136.28, 136.26, 134.44, 133.14, 130.65, 130.60, 130.59, 130.56, 129.23, 128.70, 128.59, 128.53, 128.37, 128.35, 127.41, 127.38, 125.38, 125.33, 119.21, 118.96, 113.58, 113.56, 113.55, 88.34, 87.70, 86.82, 86.74, 84.17, 84.15, 83.68, 83.65, 81.37, 81.36, 80.89, 80.86, 71.80, 71.71, 71.65, 71.54, 68.49, 68.47, 68.12, 68.10, 63.48, 63.42, 61.25, 61.23, 59.46, 59.34, 58.97, 58.84, 55.46, 55.44, 43.72, 43.64, 43.56, 24.68, 24.63, 24.60, 24.55, 24.47, 24.43, 20.68, 20.64, 20.56, 20.51, 13.99. ³¹P NMR (162 MHz, CD₃CN) δ 149.83, 149.71 ppm. HRMS calc. for C₅₁H₅₉N₇O₁₀P [M+H]⁺ 960.4061, found 960.4088.

### Lipophilic NMA Amidites

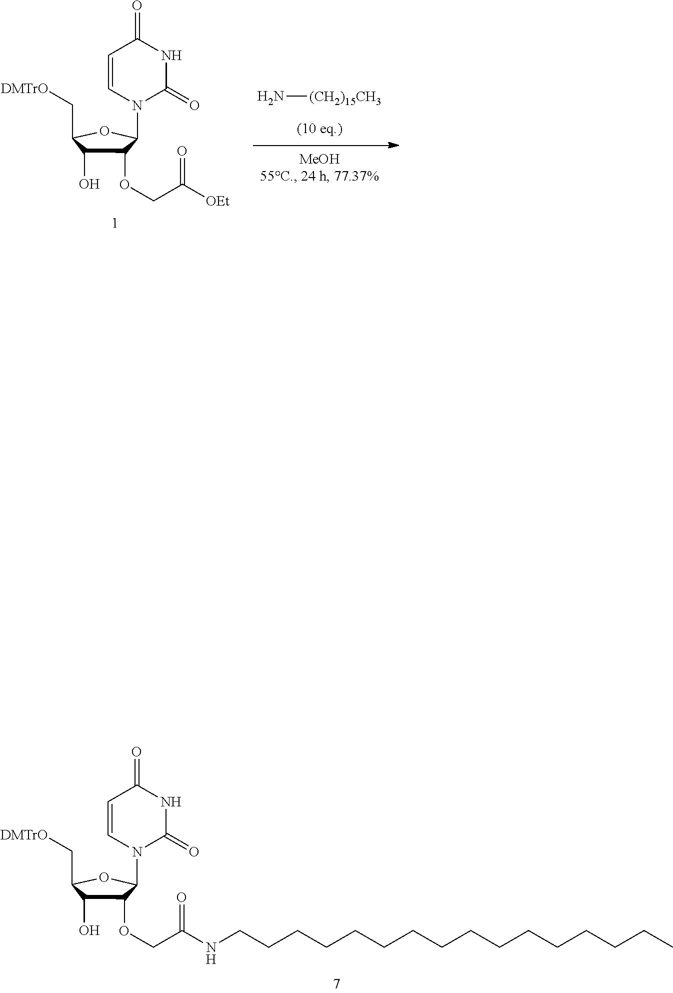

Compound 7 (ELN0069-032 and AN-0107-4): Starting material 1 (5 g, 7.90 mmol) was dissolved in MeOH (80 mL). The solution was heated to 55° C. in an oil bath and hexadecanamine (19.08 g, 79 mmol) was added. At the same temperature, the mixture was stirred for 24 hr. Upon completion of the reaction, the solvent was removed and the crude residue was purified via column chromatography (0-80% ethyl acetate in hexanes). Compound 7 was obtained as a white powder (5.06 g, 77%). ¹H NMR (400 MHz, DMSO-d₆) δ 11.40 (s, 1H), 7.88 (t, J=5.9 Hz, 1H), 7.72 (d, J=8.1 Hz, 1H), 7.48-7.13 (m, 10H), 6.90 (d, J=8.6 Hz, 4H), 5.80 (d, J=2.2 Hz, 1H), 5.56 (d, J=7.4 Hz, 1H), 5.26 (d, J=8.0 Hz, 1H), 4.28-4.11 (m, 2H), 4.07-3.97 (m, 3H), 3.74 (s, 6H), 3.26 (dd, J=10.9, 2.4 Hz, 1H), 3.09 (qd, J=7.0, 2.9 Hz, 2H), 1.40 (t, J=6.9 Hz, 2H), 1.23 (d, J=4.2 Hz, 26H), 0.85 (t, J=6.7 Hz, 3H) ppm. ¹³C NMR (126 MHz, DMSO) δ 168.62, 162.98, 158.12, 150.20, 144.54, 139.94, 135.34, 135.05, 129.71, 127.82, 127.68, 126.73, 113.20, 101.27, 87.65, 85.85, 82.26, 81.91, 69.28, 68.36, 62.21, 54.99, 38.14, 31.22, 29.04, 28.96, 28.93, 28.91, 28.66, 28.62, 26.34, 22.01, 13.84 ppm. HRMS calc. for C₄₈H₆₅N₃NaO₉ [M+Na]⁺ 850.4619, found 850.4608.

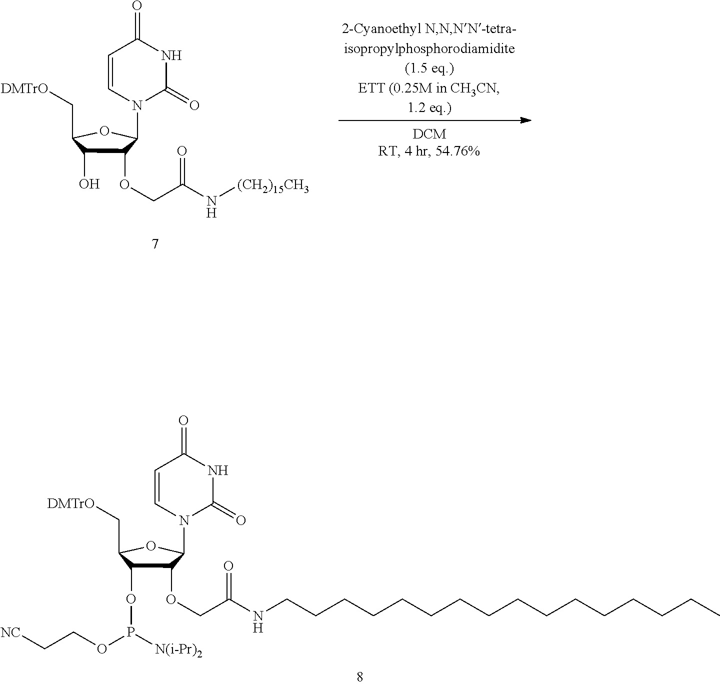

Compound 8 (AN-0107-9): To a solution of Compound 7 (500 mg, 603.83 μmol) and 5-ethylthio-1H-tetrazole (0.25 M in CH₃CN, 3.62 mL, 905.75 μmol) in CH₂Cl₂ (2 mL) were added dropwise 2-cyanoethyl-N,N,N′,N′-tetraisopropylphosphorodiamidite (0.23 mL, 724.60 μmol) at 0° C. The mixture was stirred at rt for 4 h. The reaction mixture was quenched with saturated NaHCO₃(aq.) and diluted with ethyl acetate. The organic layer was washed with saturated NaHCO₃(aq.), brine, dried (Na₂SO₄) and concentrated under vacuum. The crude residue was purification by column chromatography on silica gel (0-55% ethyl acetate in hexanes) to obtain the desired compound as a white form (340 mg, 55%). ¹H NMR (500 MHz, CD₃CN) δ 9.00 (s, 1H), 7.81-7.70 (m, 1H), 7.48-7.40 (m, 2H), 7.38-7.22 (m, 8H), 6.94-6.84 (m, 5H), 5.89 (dd, J=5.8, 2.5 Hz, 1H), 5.27-5.17 (m, 1H), 4.50 (ddd, J=9.6, 7.1, 4.9 Hz, 1H), 4.28-4.06 (m, 4H), 3.77 (d, J=3.0 Hz, 7H), 3.66-3.53 (m, 2H), 3.50 (dd, J=11.3, 2.4 Hz, 1H), 3.46-3.36 (m, 1H), 3.24-3.03 (m, 2H), 2.66 (td, J=5.8, 1.4 Hz, 1H), 2.52 (t, J=6.0 Hz, 1H), 1.49-1.42 (m, 2H), 1.33-1.20 (m, 34H), 1.20-1.10 (m, 11H), 1.03 (d, J=6.8 Hz, 2H), 0.92-0.85 (m, 4H) ppm. ¹³C NMR (151 MHz, CD₃CN) δ 169.16, 168.93, 163.52, 163.50, 159.38, 159.36, 159.35, 150.99, 150.97, 145.33, 145.26, 140.44, 140.36, 136.02, 135.94, 135.92, 135.81, 130.78, 130.75, 130.72, 128.66, 128.53, 127.60, 119.16, 118.96, 113.72, 102.12, 102.07, 89.04, 88.65, 87.23, 82.87, 82.85, 82.70, 82.39, 82.35, 81.98, 81.96, 71.20, 71.09, 70.51, 70.43, 70.30, 70.13, 70.12, 61.94, 59.07, 58.98, 58.94, 58.84, 55.52, 55.49, 43.70, 43.66, 43.62, 43.58, 39.08, 38.98, 32.23, 30.07, 30.02, 29.99, 29.97, 29.95, 29.90, 29.89, 29.87, 29.66, 29.64, 29.63, 27.24, 27.22, 27.21, 24.70, 24.65, 24.63, 24.58, 24.51, 24.47, 24.39, 24.34, 22.98, 20.67, 20.64, 20.62, 20.59, 13.99, 13.98. ³¹P NMR (202 MHz, CD₃CN) δ 151.79, 149.92 ppm. HRMS calc. for C57H₈₃N₅O₁₀P [M+H]⁺ 1028.5878, found 1028.5839.

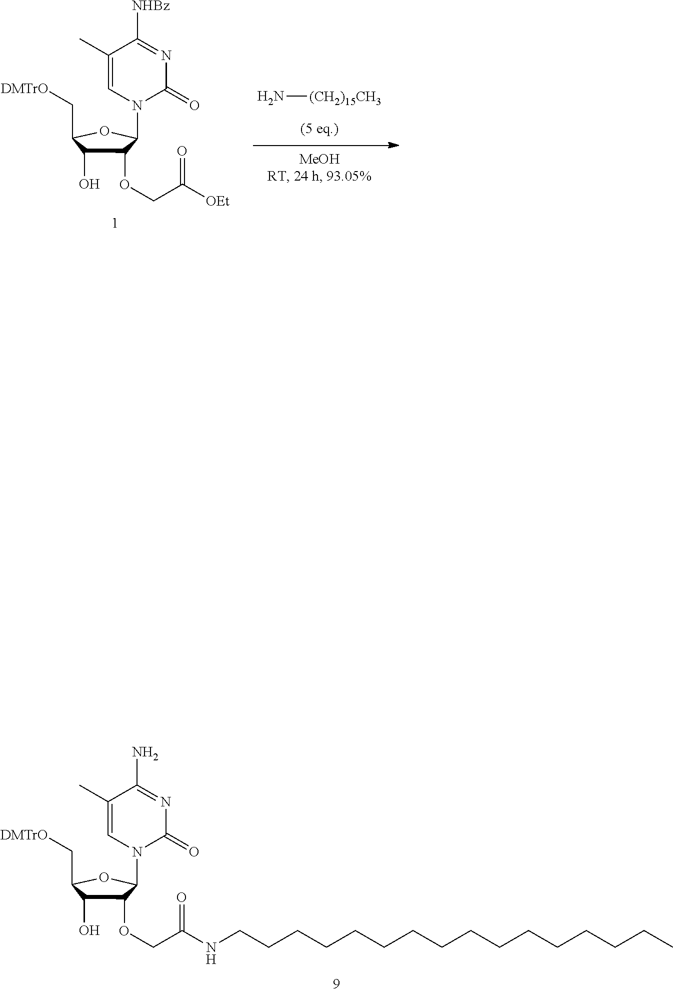

Compound 9 (ELN0069-031): Starting material 2 (5 g, 6.67 mmol) was dissolved in MeOH (65 mL). The solution was held at room temperature and hexadecanamine (8.05 g, 33.34 mmol) was added. The mixture was stirred for 24 hr. Upon completion of the reaction, the solvent was removed and the crude residue was purified via column chromatography (0-100% ethyl acetate (5% methanol) in hexanes). Compound 9 was obtained as a white powder (5.22 g, 93%). ¹H NMR (400 MHz, DMSO-d₆) δ 7.92 (t, J=5.9 Hz, 1H), 7.55-7.14 (m, 11H), 6.90 (d, J=8.5 Hz, 4H), 6.80 (s, 1H), 5.79 (d, J=2.0 Hz, 1H), 5.51 (d, J=7.3 Hz, 1H), 4.30-4.17 (m, 2H), 4.12-4.01 (m, 2H), 3.84 (dd, J=5.1, 2.1 Hz, 2H), 3.74 (s, 6H), 3.32-3.20 (m, 3H), 3.10 (q, J=6.7 Hz, 2H), 1.42 (s, 5H), 1.22 (d, J=4.1 Hz, 29H), 0.84 (t, J=6.7 Hz, 3H) ppm. ¹³C NMR (151 MHz, DMSO-d₆) δ 169.32, 165.97, 158.64, 158.63, 155.42, 145.08, 137.57, 135.94, 135.65, 130.25, 130.20, 128.65, 128.40, 128.21, 127.91, 127.27, 113.74, 101.65, 88.64, 86.22, 83.42, 81.87, 69.71, 68.87, 62.57, 55.51, 38.64, 31.77, 29.58, 29.53, 29.48, 29.47, 29.22, 29.19, 26.89, 22.57, 14.42, 13.45. HRMS calc. for C₄₉H₆₈N₄NaO₈ [M+Na]⁺ 863.4935, found 863.4956.

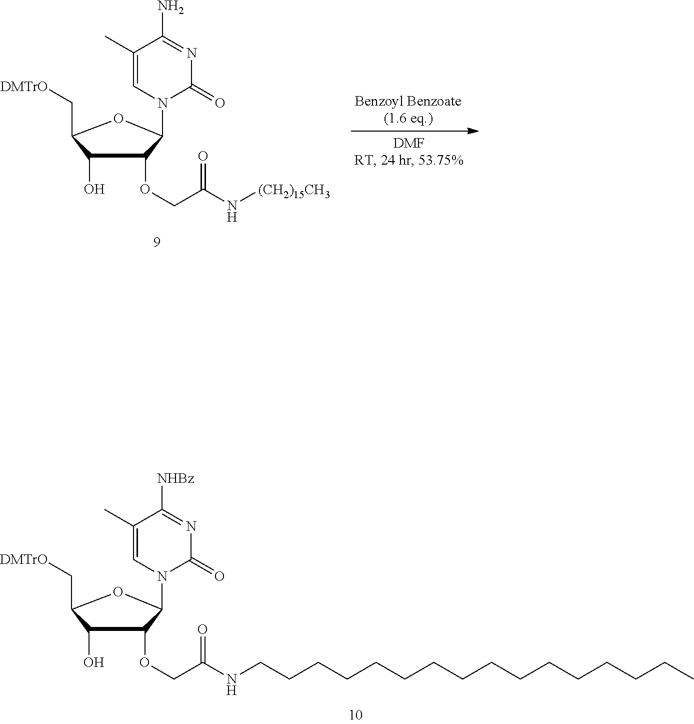

Compound 10 (AN-0107-7): Compound 9 (1 g, 1.19 mmol) was dissolved in DMF (12 mL). The solution was held at room temperature and benzoic anhydride (430.35 mg, 1.90 mmol) was added. The mixture was stirred for 24 hr. Upon completion of the reaction, the mixture was diluted with ethyl ether and washed by NaHCO₃(1×), H₂O (2×), and brine (1×) to recover the organic layer, which was then dried over Na₂SO₄. The solvent was removed from the organic phase and purified via column chromatography (0-50% ethyl acetate in hexanes). Compound 10 was obtained as a white powder (604 mg, 54%). ¹H NMR (500 MHz, DMSO-d₆) δ 13.08 (s, 1H), 8.18 (d, J=7.2 Hz, 2H), 7.88 (s, 1H), 7.78 (s, 1H), 7.58 (t, J=7.3 Hz, 1H), 7.53-7.38 (m, 4H), 7.36-7.21 (m, 7H), 6.95-6.88 (m, 4H), 5.88 (d, J=2.5 Hz, 1H), 5.61 (d, J=7.3 Hz, 1H), 4.33 (td, J=7.4, 5.1 Hz, 1H), 4.24-4.03 (m, 4H), 3.74 (d, J=0.7 Hz, 6H), 3.42-3.33 (m, 1H), 3.18-3.04 (m, 2H), 1.61 (s, 3H), 1.40 (q, J=7.1 Hz, 2H), 1.30-1.14 (m, 27H), 0.88-0.80 (m, 3H) ppm. ¹³C NMR (126 MHz, DMSO-d₆) δ 168.66, 158.20, 158.18, 147.37, 144.55, 137.77, 136.64, 135.33, 135.09, 132.48, 129.73, 129.32, 128.24, 127.91, 127.70, 126.80, 113.26, 109.92, 88.10, 85.87, 82.36, 82.17, 69.32, 68.33, 62.29, 55.01, 38.18, 31.25, 29.06, 29.00, 28.96, 28.94, 28.70, 28.66, 26.37, 22.04, 13.87 ppm. HRMS calc. for C₅₆H₇₃N₄O₉ [M+H]⁺ 945.5378, found 945.5416.

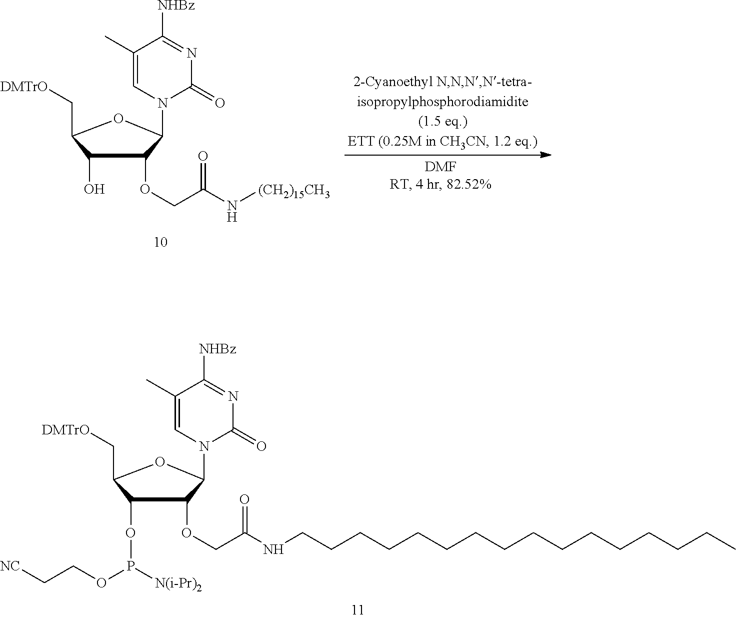

Compound 11 (AN-0107-13): To a solution of Compound 10 (500 mg, 528.99 μmol) and 5-ethylthio-1H-tetrazole (0.25 M in CH₃CN, 3.17 mL, 793.49 μmol) in CH₂Cl₂ (2 mL) were added dropwise 2-cyanoethyl-N,N,N′,N′-tetraisopropylphosphorodiamidite (0.20 mL, 634.79 μmol) at 0° C. The mixture was stirred at rt for 4 h. The reaction mixture was quenched with saturated NaHCO₃(aq.) and diluted with ethyl acetate. The organic layer was washed with saturated NaHCO₃(aq.), brine, dried (Na₂SO₄) and concentrated under vacuum. The crude residue was purification by column chromatography on silica gel (0-40% ethyl acetate in hexanes) to obtain the desired compound as a white form (500 mg, 83%). ¹H NMR (400 MHz, CD₃CN) δ 13.23 (s, 1H), 8.30-8.22 (m, 2H), 7.85-7.80 (m, 1H), 7.60-7.41 (m, 5H), 7.39-7.22 (m, 7H), 6.89 (ddd, J=8.9, 6.6, 2.3 Hz, 5H), 5.99 (dd, J=4.8, 3.1 Hz, 1H), 4.55 (tdd, J=9.3, 6.7, 5.0 Hz, 1H), 4.32 (dt, J=6.0, 2.7 Hz, 1H), 4.27 (dd, J=5.0, 3.3 Hz, 1H), 4.23-4.19 (m, 1H), 4.15 (d, J=1.6 Hz, 1H), 3.84-3.69 (m, 7H), 3.67-3.50 (m, 3H), 3.45-3.32 (m, 1H), 3.18 (tq, J=12.0, 6.1 Hz, 2H), 2.66 (t, J=5.9 Hz, 1H), 2.50 (t, J=6.0 Hz, 1H), 1.62-1.54 (m, 3H), 1.44 (p, J=7.2 Hz, 2H), 1.35-1.20 (m, 29H), 1.19-1.09 (m, 10H), 1.01 (d, J=6.8 Hz, 2H), 0.91-0.83 (m, 3H) ppm. ¹³C NMR (101 MHz, CD₃CN) δ 169.59, 169.35, 161.47, 159.93, 159.90, 149.10, 145.76, 145.71, 138.53, 138.19, 136.52, 136.44, 133.51, 131.25, 131.22, 130.62, 129.23, 129.19, 129.03, 128.13, 119.59, 119.38, 114.24, 112.28, 89.83, 89.40, 87.73, 87.70, 83.98, 83.34, 83.20, 82.42, 71.78, 71.62, 71.41, 71.29, 70.94, 70.67, 62.92, 62.83, 59.62, 59.42, 59.38, 59.17, 56.00, 55.97, 44.20, 44.08, 39.57, 39.47, 32.67, 30.49, 30.43, 30.41, 30.39, 30.35, 30.33, 30.10, 27.71, 27.68, 25.16, 25.08, 25.00, 24.95, 24.89, 24.85, 24.79, 23.42, 21.14, 21.08, 21.01, 14.42, 13.32, 13.19 ppm. 31p NMR (202 MHz, CD₃CN) δ 150.53, 148.88 ppm. HRMS calc. for C₆₅H₉₀N₆O₁₀P [M+H]⁺ 1145.6456, found 1145.6426.

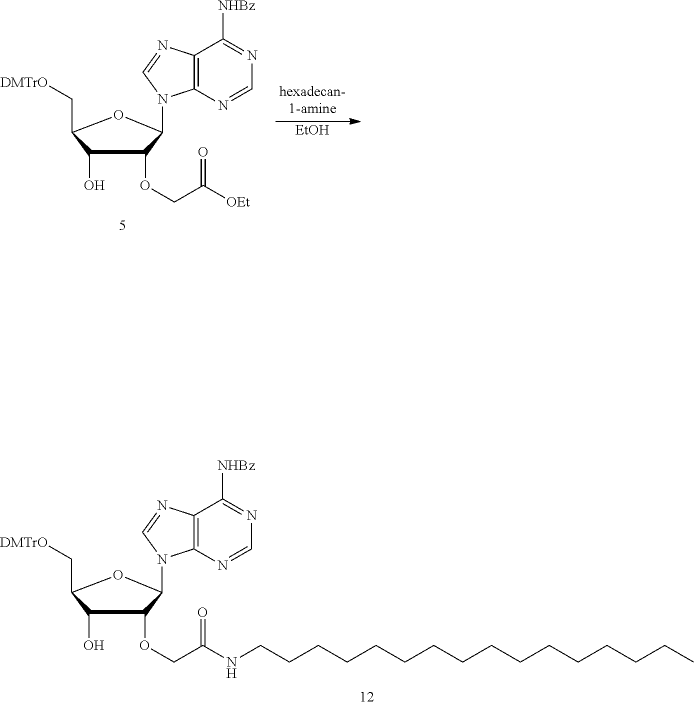

Compound 12: To a clear solution of 5 (1.4 g, 1.84 mmol) in ethanol (30 mL) was added hexadecan-1-amine (533.88 mg, 2.21 mmol) in single portions. The reaction mixture was stirred at 22° C. for 8 hr. TLC was checked and all the volatile matters were evaporated. The crude residue thus obtained was diluted with EtOAc (20 mL) and washed with saturated NH₄Cl solution (30 mL). Organic layer was then separated, dried over anhydrous Na₂SO₄, filtered and filtrated was evaporated to dryness. Solid material thus obtained, was purified by flash column chromatography (gradient: 40-90% EtOAc in hexane followed by 5% MeOH in DCM) to afford 12 (1.0 g, 57% yield) as yellowish-white foam. ¹H NMR (600 MHz, CDCl₃) δ 9.51 (s, 1H), 8.65 (s, 1H), 8.25 (s, 1H), 7.98 (d, J=7.7 Hz, 2H), 7.53 (t, J=7.4 Hz, 1H), 7.48-7.38 (m, 4H), 7.33-7.14 (m, 9H), 6.77 (d, J=8.6 Hz, 4H), 6.23 (d, J=3.2 Hz, 1H), 5.17 (s, 1H), 4.57 (t, J=4.6 Hz, 2H), 4.36 (q, J=4.0 Hz, 1H), 4.28 (d, J=15.2 Hz, 1H), 4.20 (d, J=15.3 Hz, 1H), 3.72 (s, 6H), 3.53 (dd, J=10.9, 3.2 Hz, 1H), 3.44 (dd, J=10.8, 4.2 Hz, 1H), 3.15 (p, J=6.9 Hz, 3H), 1.41 (p, J=7.0 Hz, 2H), 1.30-1.17 (m, 28H), 0.87 (t, J=6.9 Hz, 3H) ppm. ¹³C NMR (151 MHz, CDCl₃) δ 169.32, 164.96, 158.52, 152.50, 151.26, 149.55, 144.37, 141.55, 135.56, 135.48, 133.42, 132.79, 130.05, 130.01, 129.99, 128.75, 128.48, 128.13, 128.09, 127.91, 127.87, 127.34, 126.94, 123.44, 113.18, 113.12, 87.53, 86.64, 86.63, 83.95, 83.89, 70.31, 69.74, 62.90, 55.15, 39.29, 29.67, 29.65, 29.63, 29.62, 29.59, 29.59, 29.54, 29.41, 29.32, 29.27, 26.92, 14.10 ppm. HRMS calc. for C₅₆H₇₁N₆O₈ [M+H]⁺ 955.5333, found 955.5335.

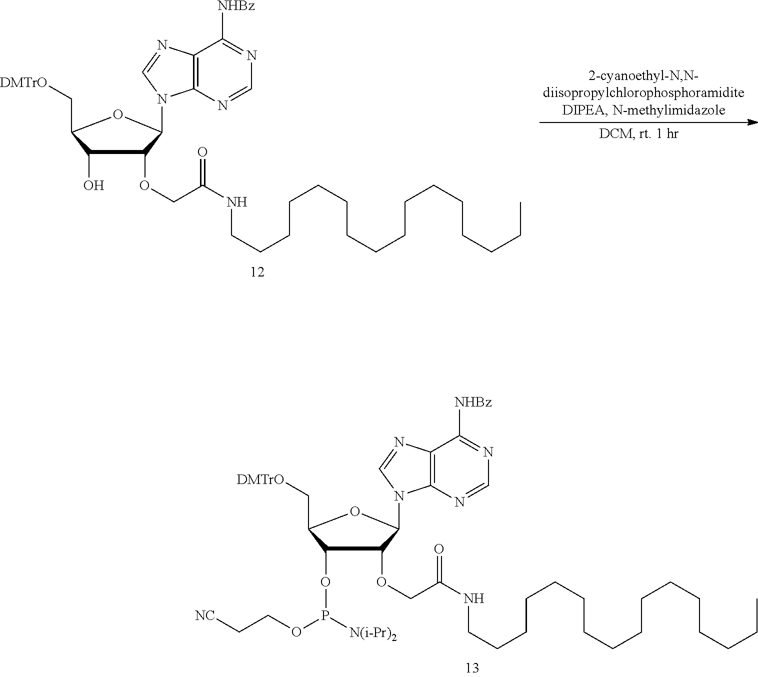

Compound 13: To a clear solution of 12 (0.6 g, 628.15 μmol) in DCM (10 mL) was added N-methylimidazole (103.14 mg, 1.26 mmol, 100.14 μL) and diisopropylethylamine (405.91 mg, 3.14 mmol, 547.05 μL) in single portions. After stirring the reaction mixture for 5 minutes at 22° C., 2-cyanoethyl-N,N-diisopropylchlorophosphoramidite (297.34 mg, 1.26 mmol, 280.51 μL) was added and continued stirring for 1 hr and TLC was checked. Starting material was consumed and reaction mixture was diluted with DCM (15 mL). DCM layer was washed with 10% NaHCO₃ (2×25 mL) solution, and brine (30 mL). Organic layer was separated, dried over anhydrous Na₂SO₄, filtered and filtrate was evaporated at 36° C. to afford crude compound which was purified by flash chromatography (30-80% EtOAc in hexane) to afford 13 (0.47 g, 65% yield) as white foam. ¹H NMR (600 MHz, CD₃CN) δ 9.34 (s, 1H), 8.59 (d, J=1.8 Hz, 1H), 8.31 (d, J=6.8 Hz, 1H), 8.01-7.97 (m, 2H), 7.66-7.60 (m, 1H), 7.53 (td, J=7.8, 1.2 Hz, 2H), 7.42-7.36 (m, 2H), 7.29-7.16 (m, 7H), 6.89-6.78 (m, 5H), 6.23 (dd, J=11.2, 4.0 Hz, 1H), 4.90-4.76 (m, 2H), 4.46-4.29 (m, 1H), 4.23-3.98 (m, 2H), 3.92-3.70 (m, 7H), 3.69-3.55 (m, 2H), 3.52-3.42 (m, 1H), 3.37-3.27 (m, 1H), 3.17-3.03 (m, 2H), 2.70-2.62 (m, 1H), 2.50 (t, J=6.0 Hz, 1H), 1.37 (h, J=7.2 Hz, 2H), 1.32-1.14 (m, 36H), 1.06 (d, J=6.8 Hz, 3H), 0.87 (t, J=7.0 Hz, 3H) ppm. ¹³C NMR (151 MHz, CD₃CN) δ 169.32, 169.16, 159.65, 159.63, 152.82, 151.01, 145.94, 145.91, 143.80, 143.69, 136.76, 136.70, 136.67, 136.65, 134.83, 133.58, 131.05, 131.01, 130.99, 129.65, 129.13, 129.00, 128.95, 128.79, 127.83, 125.88, 119.55, 119.32, 114.01, 113.99, 88.70, 88.47, 87.24, 87.18, 84.44, 84.42, 83.91, 83.88, 81.92, 81.91, 81.77, 81.74, 72.37, 72.27, 72.21, 72.13, 70.87, 70.86, 70.68, 70.66, 63.76, 63.72, 59.79, 59.67, 59.45, 59.32, 55.88, 55.86, 44.15, 44.10, 44.07, 44.02, 39.39, 39.33, 32.64, 30.43, 30.39, 30.37, 30.35, 30.31, 30.27, 30.07, 30.02, 27.60, 27.59, 25.15, 25.10, 25.04, 24.99, 24.90, 24.87, 24.85, 24.82, 23.39, 21.11, 21.07, 20.99, 20.94, 14.40 ppm. ³¹P NMR (243 MHz, CD₃CN) δ 149.82, 149.36 ppm. HRMS calc. for C₆₅H₈₈N₈O₉P [M+H]⁺ 1155.6412, found 1155.6370.

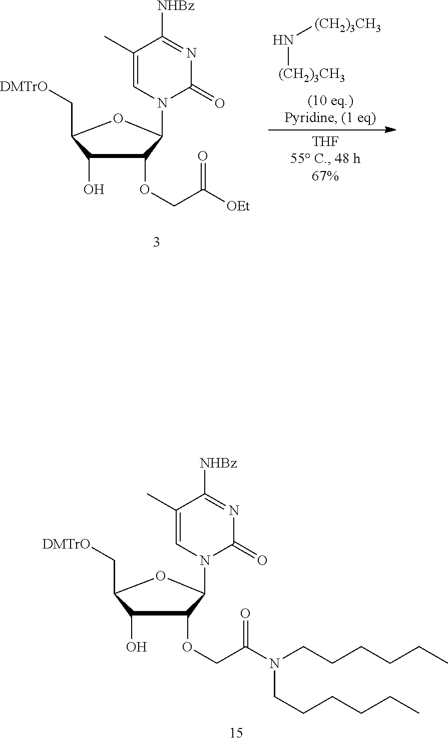

Compound 15 (AN-0107-10): Compound 3 (100 mg, 133.37 μmol) was dissolved in THF (1 mL). The solution was heated to 55° C. in an oil bath and dihexylamine (247.20 mg, 1.33 μmol) was added. The reactant was dissolved in the solution and pyridine (10.79 μL, 133.37 μmol) was added. The mixture was stirred for 48 hr. Upon completion of the reaction, the solvent was removed, and the crude product was purified via column chromatography (0-25% ethyl acetate in hexanes) to afford 15 as a white powder (70.4 mg, 67%). ¹H NMR (500 MHz, DMSO-d₆) δ 8.15 (d, J=7.6 Hz, 2H), 7.80 (s, 1H), 7.63-7.56 (m, 1H), 7.49 (dd, J=8.3, 7.1 Hz, 2H), 7.45-7.36 (m, 2H), 7.36-7.31 (m, 2H), 7.30-7.21 (m, 5H), 6.95-6.88 (m, 4H), 5.94 (d, J=3.9 Hz, 1H), 5.81 (d, J=3.9 Hz, 1H), 4.51-4.35 (m, 2H), 4.26 (d, J=4.2 Hz, 2H), 4.07 (dt, J=6.9, 3.1 Hz, 1H), 3.74 (s, 6H), 3.27-3.18 (m, 3H), 3.14 (t, J=7.8 Hz, 2H), 2.86-2.79 (m, 6H), 1.63-1.54 (m, 9H), 1.49-1.36 (m, 4H), 1.35-1.15 (m, 36H), 0.90-0.80 (m, 11H) ppm. ¹³C NMR (151 MHz, DMSO-d₆) δ 169.25, 158.69, 158.66, 145.01, 135.81, 135.53, 133.05, 130.22, 129.75, 128.79, 128.46, 128.18, 127.31, 113.78, 88.15, 86.52, 84.14, 82.95, 68.89, 68.69, 63.41, 55.51, 47.16, 46.49, 45.71, 31.45, 31.20, 28.70, 27.56, 26.53, 26.38, 26.16, 25.87, 22.54, 22.50, 22.36, 14.34, 14.31. HRMS calc. for C₅₂H₆₅N₄O₉ [M+H]⁺ 889.4752, found 889.4720.

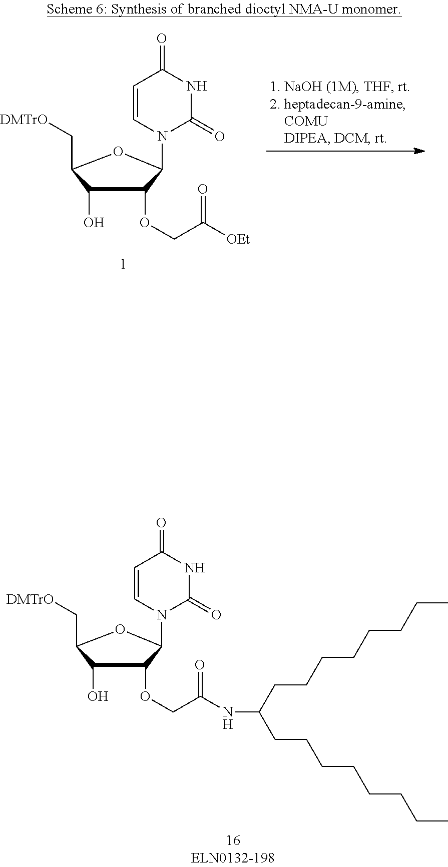

2-[(2R,5R)-5-[[bis(4-methoxyphenyl)-phenyl-methoxy]methyl]-2-(2,4-dioxopyrimidin-1-yl)-4-hydroxy-tetrahydrofuran-3-yl]oxy-N-(1-octylnonyl)acetamide 16: To a clear solution of 1 in tetrahydrofuran (20 mL) was added sodium hydroxide (54.19 mg, 1.33 mmol) dissolved in water (10 mL) and stirred for 1 hr at 22° C. Volatile matters were removed and residue was neutralized with dilute HCl. EtOAc (2×25 mL) was added to the mixture to extract the hydrolyzed compound. Organic layer was separated, dried over anhydrous Na₂SO₄ and filtered. Filtrate was evaporated to dryness and the crude mass was dissolved in DCM (30 mL) to obtain a clear solution. To the resulting solution, was added DIPEA (433.32 mg, 3.32 mmol, 584.00 μL) and heptadecan-9-amine (441.68 mg, 1.66 mmol) in single portions. Reaction mixture was cooled to 0° C. and finally COMU [(1-cyano-2-ethoxy-2-oxoethylidenaminooxy)dimethylamino-morpholino-carbenium hexafluorophosphate](732.77 mg, 1.66 mmol) was added. Ice bath was removed, and reaction mixture was stirred for 15 hrs at 22° C. Reaction mixture was diluted with DCM (30 mL) and organic layer was washed with water (30 mL) and brine (40 mL). DCM layer was separated, dried over anhydrous Na₂SO₄, filtered and the filtrate was evaporated to dryness. Crude compound was purified by combiflash chromatography (gradient: 0-7% MeOH in DCM) to afford 16 (0.61 g, 66% yield) as white solid. ¹H NMR (500 MHz, CDCl₃) δ 9.95 (s, 2H), 8.03 (dd, J=8.1, 1.7 Hz, 1H), 7.42-7.35 (m, 2H), 7.35-7.14 (m, 10H), 6.88-6.80 (m, 5H), 6.50 (s, 1H), 5.87-5.77 (m, 1H), 5.34 (d, J=8.1 Hz, 1H), 4.44-4.25 (m, 5H), 4.19-3.94 (m, 2H), 3.89 (d, J=5.4 Hz, 2H), 3.82-3.77 (m, 7H), 3.64-3.52 (m, 2H), 1.65 (q, J=9.0 Hz, 1H), 1.47 (d, J=7.5 Hz, 2H), 1.40-1.09 (m, 35H), 0.86 (td, J=6.9, 2.9 Hz, 7H) ppm. ¹³C NMR (101 MHz, CDCl₃) δ 169.58, 163.39, 159.43, 158.85, 158.81, 158.75, 157.82, 151.06, 144.59, 142.88, 139.87, 139.59, 135.46, 135.26, 134.45, 130.96, 130.32, 130.22, 129.27, 129.03, 128.27, 128.15, 128.10, 128.06, 127.98, 127.90, 127.24, 127.21, 125.60, 113.46, 113.44, 113.32, 113.29, 108.56, 102.44, 97.36, 89.11, 87.18, 84.97, 83.37, 70.00, 68.11, 63.26, 61.12, 60.56, 55.39, 49.68, 34.99, 32.00, 31.98, 31.80, 29.72, 29.69, 29.50, 29.45, 29.43, 29.41, 29.18, 29.09, 26.11, 26.05, 22.80, 22.73, 14.25, 14.19, 14.10 ppm. HRMS calc. for C₄₉H₆₇N₃O₉Na [M+Na]⁺ 864.4775, found 864.4768.

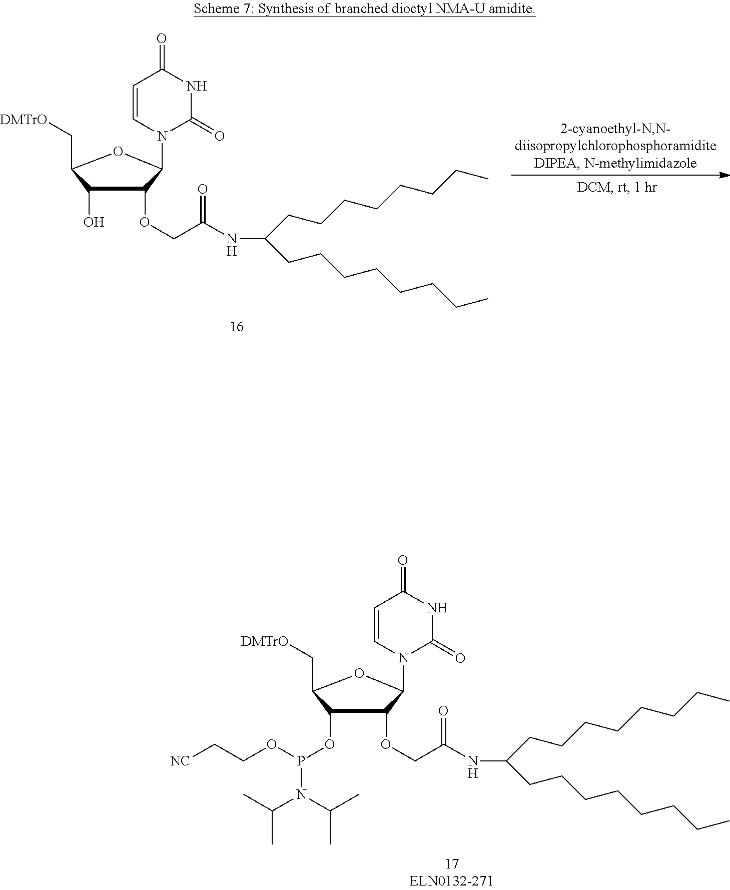

2-[(2R,5R)-5-[[bis(4-methoxyphenyl)-phenyl-methoxy]methyl]-4-[2-cyanoethoxy-(diisopropylamino)phosphanyl]oxy-2-(2,4-dioxopyrimidin-1-yl)tetrahydrofuran-3-yl]oxy-N-(1-octylnonyl)acetamide 17: To a clear solution of 16 (0.5 g, 593.78 μmol) in DCM (20 mL) at 22° C. was added N-methyl imidazole (73.86 mg, 890.66 mol, 71.71 μL) and DIPEA (387.57 mg, 2.97 mmol, 522.34 μL). The reaction mixture was stirred for 5 minutes at 22° C. and 2-cyanoethyl-N,N-diisopropylchlorophosphoramidite (295.86 mg, 1.19 mmol, 279.12 μL) was added slowly into it. Reaction was kept for stirring at 22° C. and TLC was checked after 1 hr. Reaction mixture was diluted with DCM (20 mL) and washed with 10% NaHCO₃ solution (2×30 mL). Organic layer separated, dried over anhydrous Na₂SO₄, filtered and the filtrate was evaporated to dryness. The crude mass obtained was purified by combiflash chromatography (gradient: 20-60% EtOAc in hexane) to afford 17 (0.44 g, 71% yield) as white foam. ¹H NMR (400 MHz, CD₃CN) δ 9.07 (s, 1H), 7.49-7.40 (m, 2H), 7.35-7.25 (m, 7H), 6.93-6.83 (m, 5H), 6.50 (dd, J=13.7, 9.3 Hz, 1H), 5.91 (dd, J=6.5, 3.0 Hz, 1H), 5.25 (dd, J=17.7, 8.2 Hz, 1H), 4.48 (ddd, J=7.0, 3.4, 1.9 Hz, 1H), 4.34-4.06 (m, 4H), 3.89-3.70 (m, 8H), 3.74-3.35 (m, 5H), 2.82-2.61 (m, 1H), 2.52 (t, J=6.0 Hz, 1H), 1.56-1.08 (m, 44H), 1.04 (d, J=6.8 Hz, 3H), 0.93-0.80 (m, 8H) ppm. ¹³C NMR (101 MHz, CD₃CN) δ 169.20, 168.99, 163.79, 163.77, 159.84, 151.42, 145.77, 145.69, 140.79, 140.71, 136.48, 136.40, 136.28, 131.22, 131.19, 131.16, 129.10, 128.97, 128.05, 119.52, 119.36, 114.17, 102.65, 89.33, 88.93, 87.70, 83.59, 83.26, 83.02, 82.58, 71.84, 71.68, 71.34, 71.23, 70.82, 70.50, 62.61, 59.61, 59.42, 59.22, 55.96, 55.94, 49.51, 44.17, 44.04, 35.94, 35.81, 32.63, 32.57, 30.30, 30.26, 30.22, 30.05, 30.01, 27.49, 26.87, 26.85, 26.83, 26.77, 25.22, 25.14, 25.04, 24.94, 24.88, 24.84, 24.78, 23.39, 21.07, 14.42, 14.39 ppm. ³¹P NMR (162 MHz, CD₃CN) δ 150.70, 149.27 ppm. HRMS calc. for C₅₈H₈₄N₅O₁₀PNa [M+Na]⁺ 1064.5854, found 1064.5851.

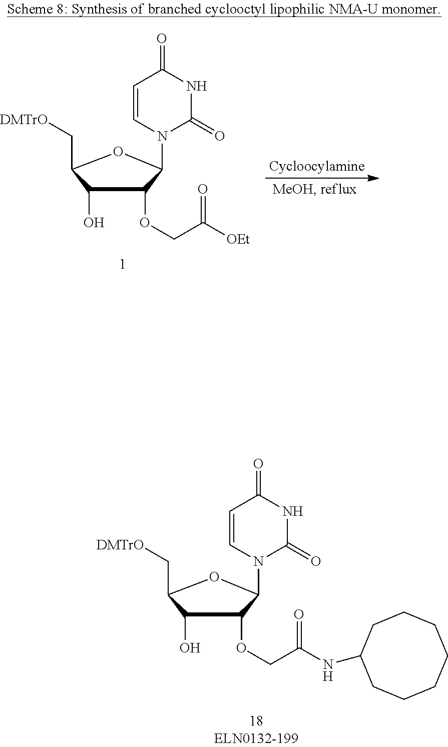

2-[(2R,5R)-5-[[bis(4-methoxyphenyl)-phenyl-methoxy]methyl]-2-(2,4-dioxopyrimidin-1-yl)-4-hydroxy-tetrahydrofuran-3-yl]oxy-N-cyclooctyl-acetamide 18: To a clear solution of 1 (0.89 g, 1.41 mmol) in methanol (30 mL) was added cyclooctylamine (369.04 mg, 2.81 mmol, 397.67 μL) in single portion. The reaction mixture was stirred under refluxing condition for 16 hr. TLC was checked and all the volatile matters were evaporated. The crude residue thus obtained was diluted with EtOAc (20 mL) and washed with saturated NH₄Cl solution (30 mL). Organic layer was then separated, dried over anhydrous Na₂SO₄, filtered and filtrated was evaporated to dryness. Solid material thus obtained, was purified by combiflash chromatography (Gradient: 40-100% EtOAc in hexane) to afford 18 (0.8 g, 80% yield) as white solid. ¹H NMR (400 MHz, DMSO) δ 11.38 (d, J=2.2 Hz, 1H), 7.74 (dd, J=14.6, 8.1 Hz, 2H), 7.47-7.15 (m, 9H), 6.94-6.85 (m, 4H), 5.80-5.72 (m, 2H), 5.23 (dd, J=8.1, 2.2 Hz, 1H), 4.22 (td, J=7.1, 5.1 Hz, 1H), 4.13 (d, J=15.6 Hz, 1H), 4.07-3.97 (m, 3H), 3.82 (dt, J=7.9, 3.4 Hz, 1H), 3.73 (s, 6H), 3.31 (qd, J=10.9, 3.4 Hz, 3H), 1.66-1.33 (m, 12H) ppm. ¹³C NMR (126 MHz, DMSO) δ 170.29, 167.63, 163.06, 158.14, 150.24, 144.58, 139.96, 135.34, 135.04, 129.78, 127.89, 127.72, 126.79, 113.24, 101.25, 87.80, 85.91, 82.62, 82.02, 69.24, 68.13, 62.17, 59.72, 55.04, 54.88, 48.32, 31.65, 26.70, 25.02, 23.37, 23.31, 20.73, 14.06 ppm. HRMS calc. for C₄₀H₄₇N₃O₉Na [M+Na]⁺ 736.3210, found 736.3209.

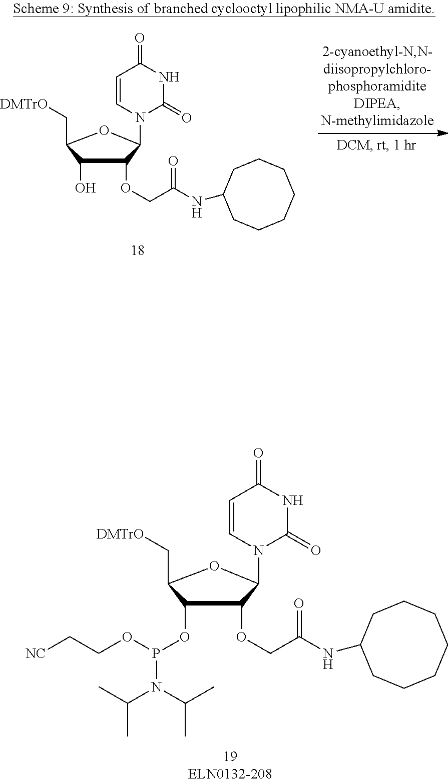

2-[(2R,5R)-5-[[bis(4-methoxyphenyl)-phenyl-methoxy]methyl]-4-[2-cyanoethoxy-(diisopropylamino)phosphanyl]oxy-2-(2,4-dioxopyrimidin-1-yl)tetrahydrofuran-3-yl]oxy-N-cyclooctyl-acetamide 19: To a clear solution of 18 (0.7 g, 980.65 μmol) in dry DCM (15 mL) at 22° C. was added, DIPEA (640.10 mg, 4.90 mmol, 862.66 μL) and N-methylimidazole (121.99 mg, 1.47 mmol, 118.43 μL) slowly. The resulting solution was stirred for 5 minutes after which 2-cyanoethyl-N,N-diisopropylchlorophosphoramidite (488.63 mg, 1.96 mmol, 460.97 μL) was added in single portion. Reaction mixture was kept for 0.5 hr stirring at 22° C. and TLC was checked. Reaction mixture was diluted with DCM (50 mL) and washed with 10% sodium bicarbonate solution (50×2 mL). Organic layer separated, dried over anhydrous Na₂SO₄, filtered and the filtrate was evaporated to dryness. The crude mass obtained was purified by combiflash chromatography (gradient: 20-50% EtOAc in hexane) to afford 19 (0.61 g, 68% yield) as white foam. ¹H NMR (400 MHz, CD₃CN) δ 9.04 (s, 1H), 7.79-7.67 (m, 1H), 7.48-7.23 (m, 9H), 6.93-6.83 (m, 4H), 6.71 (t, J=9.4 Hz, 1H), 5.92 (dd, J=7.5, 3.4 Hz, 1H), 5.27 (dd, J=15.0, 8.1 Hz, 1H), 4.46 (dtd, J=9.8, 6.4, 5.0 Hz, 1H), 4.26-4.04 (m, 4H), 3.90-3.70 (m, 8H), 3.73-3.33 (m, 4H), 2.72-2.63 (m, 1H), 2.52 (t, J=6.0 Hz, 1H), 1.77-1.43 (m, 9H), 1.29-1.10 (m, 14H), 1.04 (d, J=6.8 Hz, 3H) ppm. ¹³C NMR (126 MHz, CD₃CN) δ 168.32, 168.14, 163.96, 163.94, 159.84, 159.82, 151.46, 145.76, 145.68, 140.88, 140.82, 136.46, 136.40, 136.28, 131.22, 131.18, 131.16, 129.10, 129.08, 128.97, 128.04, 119.57, 119.37, 114.19, 102.72, 89.05, 88.67, 87.76, 87.74, 83.76, 83.73, 83.33, 83.29, 83.11, 82.55, 82.52, 71.85, 71.72, 71.37, 71.27, 70.85, 70.57, 70.55, 62.86, 62.75, 59.66, 59.51, 59.41, 59.25, 55.97, 55.95, 55.33, 49.86, 49.82, 44.17, 44.15, 44.07, 44.05, 33.18, 33.16, 33.02, 27.92, 27.87, 26.32, 26.26, 25.15, 25.09, 25.06, 25.00, 24.93, 24.88, 24.85, 24.80, 24.65, 24.63, 24.55, 21.10, 21.05, 21.00 ppm. ³¹P NMR (162 MHz, CD₃CN) δ 150.41, 149.12 ppm. HRMS calc. for C₄₉H₆₄N₅O₁₀PNa [M+Na]⁺ 936.4289, found 936.4299.

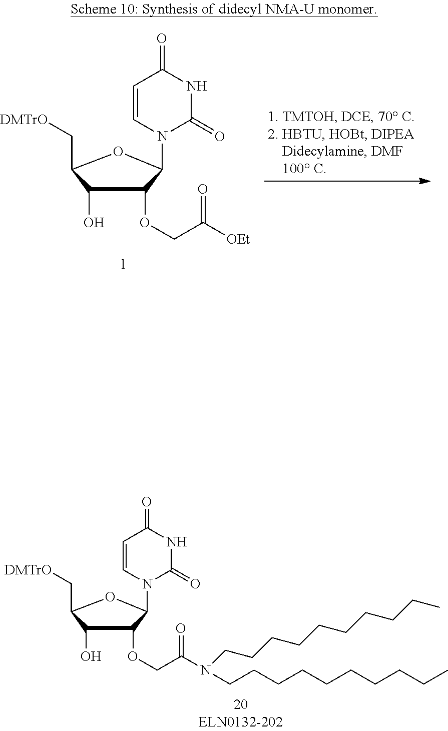

2-[(2R,5R)-5-[[bis(4-methoxyphenyl)-phenyl-methoxy]methyl]-2-(2,4-dioxopyrimidin-1-yl)-4-hydroxy-tetrahydrofuran-3-yl]oxy-N,N-didecyl-acetamide 20: To a clear solution of 1(0.4 g, 632.26 μmol) in dry dichloroethane (30 mL) was added trimethyltin hydroxide (583.29 mg, 3.16 mmol) and stirred at 70° C. for 5 hrs. TLC was checked and all the volatile matters were evaporated to dryness. Crude mass was purified through flash chromatography (gradient: 0-7% MeOH in DCM) and subjected to next step. The dried, pure fraction was dissolved in dry dimethylformamide (15.0 mL) and to the mixture was added HBTU (290.64 mg, 758.71 μmol) and 1-hydroxybenzotriazole hydrate (117.36 mg, 758.71 μmol) in single portions. To the resulting mixture DIPEA (247.61 mg, 1.90 mmol, 333.71 μL) was added and stirred for 5 minutes. Finally, didecylamine (230.37 mg, 758.71 μmol) was added and the reaction mixture was stirred for 15 hrs at 22° C. Reaction mixture was diluted with EtOAc (30 mL) and brine (30 mL). Organic layer was separated, and aqueous layer was washed with EtOAc (20 mL). Combined organic layer was washed with NaHCO₃ solution (2×30 mL), NH₄Cl (2×30 mL) and brine (3×30 mL). EtOAc layer was dried over anhydrous Na₂SO₄, filtered and the filtrate was evaporated to dryness. Crude mass was purified by column chromatography (gradient: 20-70% EtOAc in hexane) to afford 20 (0.21 g, 38% yield) as white solid. ¹H NMR (400 MHz, DMSO) δ 11.38 (s, 1H), 7.68 (d, J=8.1 Hz, 1H), 7.40-7.27 (m, 4H), 7.27-7.19 (m, 5H), 6.94-6.85 (m, 4H), 5.86 (d, J=4.2 Hz, 1H), 5.79 (d, J=4.4 Hz, 1H), 5.32 (d, J=8.0 Hz, 1H), 4.46 (d, J=15.2 Hz, 1H), 4.35 (d, J=15.2 Hz, 1H), 4.14 (dt, J=11.9, 4.9 Hz, 2H), 3.99 (dt, J=7.6, 3.5 Hz, 1H), 3.74 (s, 6H), 3.31-3.09 (m, 6H), 1.52-1.38 (m, 5H), 1.22 (s, 31H), 0.84 (t, J=6.7 Hz, 6H) ppm. ¹³C NMR (126 MHz, DMSO) δ 168.82, 162.94, 158.12, 150.40, 144.54, 140.30, 135.33, 135.08, 129.71, 127.86, 127.69, 126.74, 113.22, 101.55, 87.04, 85.92, 83.05, 82.37, 68.45, 68.36, 62.95, 55.01, 46.01, 45.22, 31.25, 28.94, 28.91, 28.71, 28.68, 28.64, 28.19, 27.06, 26.32, 26.17, 22.06, 13.90 ppm. HRMS calc. for C₅₂H₇₃N₃O₉Na [M+Na]⁺ 906.5245, found 906.5236.

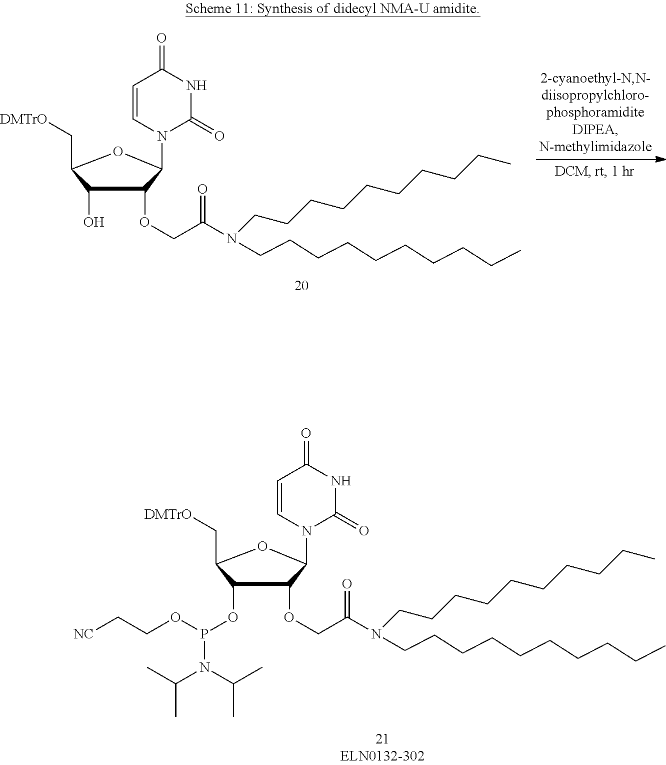

2-[(2R,5R)-5-[[bis(4-methoxyphenyl)-phenyl-methoxy]methyl]-4-[2-cyanoethoxy-(diisopropylamino)phosphanyl]oxy-2-(2,4-dioxopyrimidin-1-yl)tetrahydrofuran-3-yl]oxy-N,N-didecyl-acetamide 21: To a clear solution of 20 (0.27 g, 305.38 μmol) in DCM (15 mL) at 22° C. was added N-methyl imidazole (50.65 mg, 610.76 mol, 49.17 μL) and DIPEA (199.33 mg, 1.53 mmol, 268.64 μL). The reaction mixture was stirred for 5 minutes at 22° C. and 2-cyanoethyl-N,N-diisopropylchlorophosphoramidite (152.16 mg, 610.76 mol, 143.55 μL) was added slowly into it. Reaction was kept for stirring at 22° C. and TLC was checked after 1 hr. Reaction mixture was diluted with DCM (20 mL) and washed with 10% NaHCO₃ solution (2×30 mL). Organic layer separated, dried over anhydrous Na₂SO₄, filtered and the filtrate was evaporated to dryness. The crude mass obtained was purified by combiflash chromatography (gradient: 20-60% EtOAc in hexane) to afford 21 (0.26 g, 79% yield) as yellow gum. ¹H NMR (400 MHz, CD₃CN) δ 9.11 (s, 1H), 7.69 (d, J=8.1 Hz, 1H), 7.47-7.38 (m, 2H), 7.37-7.20 (m, 7H), 6.92-6.82 (m, 4H), 5.98 (dd, J=10.8, 5.0 Hz, 1H), 5.29 (dd, J=8.1, 6.0 Hz, 1H), 4.57-4.46 (m, 1H), 4.43-4.28 (m, 3H), 4.26-4.12 (m, 1H), 3.94-3.71 (m, 8H), 3.69-3.52 (m, 2H), 3.43-3.09 (m, 6H), 2.73-2.62 (m, 1H), 2.50 (t, J=6.1 Hz, 1H), 1.62-1.38 (m, 5H), 1.32-1.14 (m, 41H), 1.05-0.97 (m, 4H), 0.91-0.83 (m, 6H) ppm. ¹³C NMR (101 MHz, CD₃CN) δ 168.63, 168.58, 163.84, 159.79, 159.78, 151.50, 151.47, 145.70, 145.64, 141.56, 141.39, 136.51, 136.46, 136.42, 136.34, 131.19, 131.12, 131.10, 129.12, 129.06, 128.97, 127.99, 119.60, 119.34, 114.17, 102.74, 102.70, 88.42, 88.03, 87.77, 87.74, 84.18, 84.15, 83.88, 83.84, 81.99, 81.96, 81.30, 81.25, 71.93, 71.80, 71.68, 71.51, 69.72, 69.68, 69.33, 63.70, 63.36, 60.01, 59.83, 59.27, 59.06, 55.95, 55.94, 47.65, 47.58, 46.33, 44.20, 44.08, 43.95, 32.65, 30.34, 30.31, 30.16, 30.12, 30.07, 29.72, 28.40, 27.71, 27.62, 27.60, 25.19, 25.11, 25.08, 25.01, 24.95, 24.88, 23.41, 21.11, 21.05, 21.03, 20.96, 14.42 ppm. ³¹P NMR (162 MHz, CD₃CN) δ 151.02, 150.85 ppm. HRMS calc. for C₆₁H₉₀N₅O₁₀P [M+H]⁺ 1084.6504, found 1084.6501.

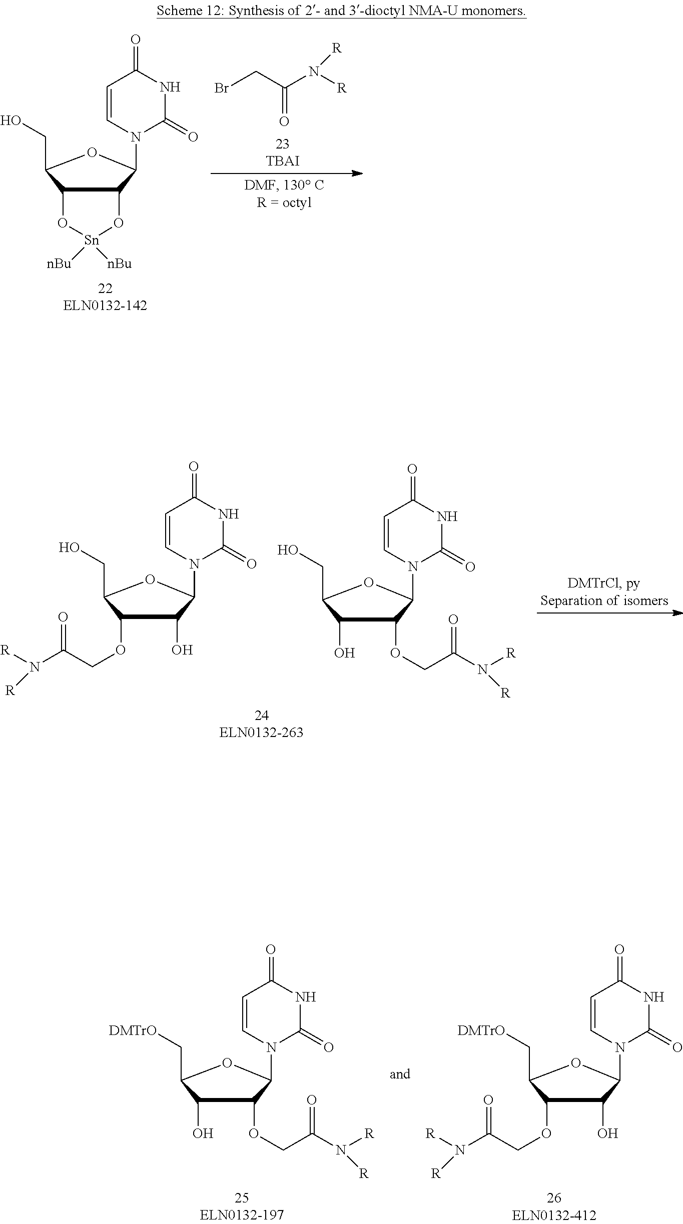

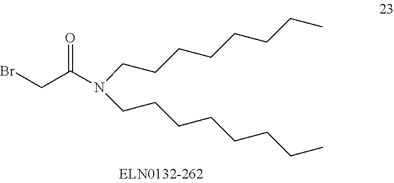 [SMILES: CCCCCCCCN(CCCCCCCC)C(=O)CBr]

2-bromo-N,N-dioctyl-acetamide⁵ [23]: To a stirred solution of 2-bromoacetic acid (2.89 g, 20.15 mmol) in THF (100 mL) under argon, was added dropwise triethylamine (6.15 g, 60.45 mmol, 8.47 mL). The reaction mixture was cooled to 0° C. and trimethylacetyl chloride (2.45 g, 20.15 mmol, 2.51 mL) was introduced dropwise. After 4 hrs of stirring at 0° C., lithium chloride (862.85 mg, 20.15 mmol, 416.83 μL) and dioctylamine (5.02 g, 20.15 mmol, 6.28 mL) were added. Then the reaction was warmed to 22° C., stirred overnight and quenched with an aqueous solution of HCl (1.0 M). The organic phase was extracted with EtOAc and washed with NaOH (1.0 M). The organic layer was dried over anhydrous Na₂SO₄, filtered, and concentrated under reduced pressure. Purification by flash chromatography to afford 2-bromo-N,N-dioctyl-acetamide 23 (5.78 g, 79% yield) as yellow oil. ¹H NMR (500 MHz, CDCl₃) δ 3.81 (s, 2H), 3.31-3.20 (m, 4H), 1.66-1.44 (m, 4H), 1.27 (tt, J=11.9, 6.1 Hz, 22H), 0.85 (q, J=6.8 Hz, 6H) ppm. ¹³C NMR (101 MHz, CDCl₃) δ 166.29, 48.87, 46.22, 31.85, 31.80, 29.40, 29.33, 29.27, 29.24, 27.30, 26.93, 26.61, 22.69, 22.67, 14.14, 14.12 ppm. HRMS calc. for C₁₈H₃₆BrNO [M+H]⁺ 362.2059, found 362.2053.

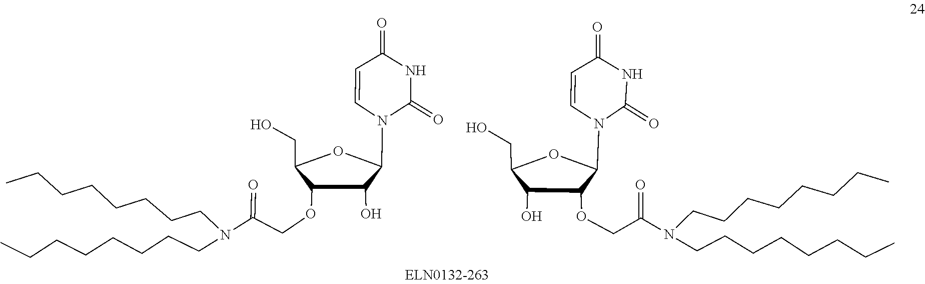

2-[(2R,5R)-5-(2,4-dioxopyrimidin-1-yl)-4-hydroxy-2-(hydroxymethyl)tetrahydrofuran-3-yl]oxy-N,N-dioctyl-acetamide and 2-[(2R,5R)-2-(2,4-dioxopyrimidin-1-yl)-4-hydroxy-5-(hydroxymethyl)tetrahydrofuran-3-yl]oxy-N,N-dioctyl-acetamide [24]: To a solution of 2′,3′-O-dibutylstannylene uridine⁶ [22](0.5 g, 1.05 mmol) in dimethylformamide (20 mL) was added tetrabutylammonium iodide (951.94 mg, 2.53 mmol) and 2-bromo-N,N-dioctyl-acetamide [23](933.95 mg, 2.53 mmol) in single portions. The resulting mixture was heated to reflux at 130° C. for 12 hr. DMF of the resulting red colored solution was removed under high vacuum to obtain a gummy brown mass which was purified by combiflash (gradient: 0-10% MeOH in DCM) to afford a mixture of 2′- and 3′-isomers [24](0.258 g, 47% yield) and as brownish gum. ¹H NMR (400 MHz, DMSO) δ 11.32 (dd, J=5.4, 2.3 Hz, 1H), 7.87 (dd, J=8.2, 7.3 Hz, 1H), 5.89 (dd, J=5.0, 2.0 Hz, 1H), 5.74 (dd, J=7.1, 3.8 Hz, 1H), 5.63 (ddd, J=8.1, 6.9, 2.2 Hz, 1H), 5.11 (q, J=4.5 Hz, 1H), 4.57-4.19 (m, 2H), 4.12-3.93 (m, 2H), 3.92-3.85 (m, 1H), 3.63-3.50 (m, 1H), 3.24-3.05 (m, 7H), 1.62-1.54 (m, 2H), 1.43 (dd, J=15.2, 7.8 Hz, 5H), 1.31-1.15 (m, 24H), 0.92 (t, J=7.3 Hz, 4H), 0.88-0.80 (in, 6H) ppm. ¹³C NMR (101 MHz, DMSO) δ 169.25, 169.11, 163.06, 163.04, 150.64, 150.59, 140.53, 101.89, 101.65, 88.13, 86.01, 85.18, 83.06, 82.88, 79.43, 72.78, 68.63, 68.50, 68.42, 60.82, 60.47, 57.55, 57.52, 45.99, 45.26, 45.21, 31.20, 28.71, 28.69, 28.65, 28.62, 28.21, 28.13, 27.06, 26.34, 26.20, 23.06, 22.05, 19.21, 13.92, 13.47 ppm. HRMS calc. for C₂₇H₄₈N₃O₇ [M+H]⁺ 526.3492, found 526.3488.

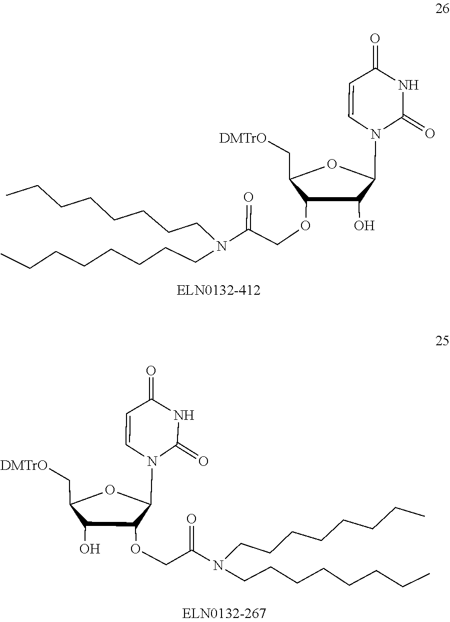

2-[(2R,5R)-5-[[bis(4-methoxyphenyl)-phenyl-methoxy]methyl]-2-(2,4-dioxopyrimidin-1-yl)-4-hydroxy-tetrahydrofuran-3-yl]oxy-N,N-dioctyl-acetamide [25] and 2-[(2R,5R)-2-[[bis(4-methoxyphenyl)-phenyl-methoxy]methyl]-5-(2,4-dioxopyrimidin-1-yl)-4-hydroxy-tetrahydrofuran-3-yl]oxy-N,N-dioctyl-acetamide [26]: To a clear solution of 24 (1.9 g, 3.61 mmol) in dry pyridine (30 mL) was added 4,4′-dimethoxytrityl chloride (1.52 g, 4.34 mmol) in three portions. Reaction mixture was stirred for 16 hrs at 22° C. and then quenched with saturated NaHCO₃ solution (30 mL). Resultant mixture was extracted with DCM (2×40 mL). The combined organic layer was separated, washed with brine (40 mL), dried over anhydrous Na₂SO₄, filtered and the filtrate was evaporated to dryness. Crude compound was purified by combiflash chromatography (gradient: 20-70% EtOAc in hexane) to afford 25 (1.0 g, 56% yield) and 26 (0.5 g, 42% yield) as yellowish white foam.

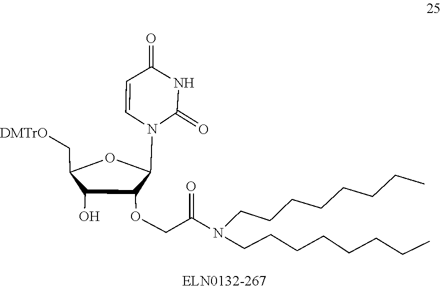

Data for 2-[(2R,5R)-5-[[bis(4-methoxyphenyl)-phenyl-methoxy]methyl]-2-(2,4-dioxopyrimidin-1-yl)-4-hydroxy-tetrahydrofuran-3-yl]oxy-N,N-dioctyl-acetamide 25: ¹H NMR (600 MHz, DMSO-d₆) δ 11.39 (s, 1H), 7.68 (d, J=8.1 Hz, 1H), 7.37 (d, J=7.8 Hz, 2H), 7.32 (t, J=7.6 Hz, 2H), 7.26-7.20 (m, 6H), 6.90 (d, J=8.5 Hz, 4H), 5.86 (d, J=4.4 Hz, 1H), 5.79 (d, J=4.5 Hz, 1H), 5.32 (d, J=8.0 Hz, 1H), 4.46 (d, J=15.2 Hz, 1H), 4.35 (d, J=15.3 Hz, 1H), 4.14 (dt, J=17.2, 4.9 Hz, 2H), 3.99-3.95 (m, 1H), 3.74 (s, 6H), 3.28-3.18 (m, 4H), 3.13 (t, J=7.9 Hz, 2H), 1.50-1.38 (m, 4H), 1.29-1.15 (m, 21H), 0.84 (t, J=6.8 Hz, 6H) ppm. ¹³C NMR (151 MHz, DMSO-d₆) δ 168.84, 162.97, 158.13, 150.42, 144.56, 140.35, 135.35, 135.09, 129.74, 127.90, 127.70, 126.77, 113.25, 113.24, 101.57, 87.05, 85.93, 83.08, 82.35, 68.46, 68.35, 62.98, 59.74, 55.03, 54.91, 46.00, 45.21, 31.20, 28.70, 28.67, 28.63, 28.61, 28.21, 27.08, 26.35, 26.21, 22.04, 13.92 ppm. HRMS calc. for C₄₈H₆₆N₃O₉ [M+H]⁺ 828.4799

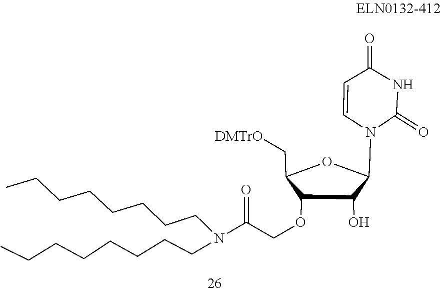

Data for 2-[(2R,5R)-2-[[bis(4-methoxyphenyl)-phenyl-methoxy]methyl]-5-(2,4-dioxopyrimidin-1-yl)-4-hydroxy-tetrahydrofuran-3-yl]oxy-N,N-dioctyl-acetamide 26: ¹H NMR (600 MHz, DMSO-d₆) δ 11.41 (t, J=1.9 Hz, 1H), 7.43-7.19 (m, 10H), 6.89 (d, J=8.4 Hz, 4H), 6.03 (d, J=3.9 Hz, 1H), 5.73 (d, J=3.3 Hz, 1H), 5.30 (dt, J=8.0, 1.6 Hz, 1H), 4.42-4.33 (m, 2H), 4.17 (q, J=4.0 Hz, 1H), 4.13-4.05 (m, 2H), 3.74 (s, 6H), 3.33-3.23 (m, 2H), 3.21 (t, J=7.6 Hz, 2H), 3.11 (t, J=7.8 Hz, 2H), 1.51-1.39 (m, 5H), 1.21 (q, J=10.3 Hz, 23H), 0.83 (dt, J=11.2, 6.7 Hz, 6H) ppm. ¹³C NMR (151 MHz, DMSO-d₆) δ 169.12, 163.06, 158.16, 158.14, 150.41, 144.70, 140.51, 135.36, 135.14, 129.83, 129.79, 127.94, 127.75, 126.81, 113.28, 101.42, 89.12, 86.00, 80.61, 79.00, 72.40, 68.65, 62.36, 55.06, 55.05, 46.11, 45.26, 31.26, 31.24, 28.77, 28.75, 28.68, 28.26, 27.11, 26.40, 26.26, 22.11, 22.09, 13.99, 13.97 ppm. HRMS calc. for C₄₈H₆₆N₃O₉ [M+H]⁺ 828.4799.

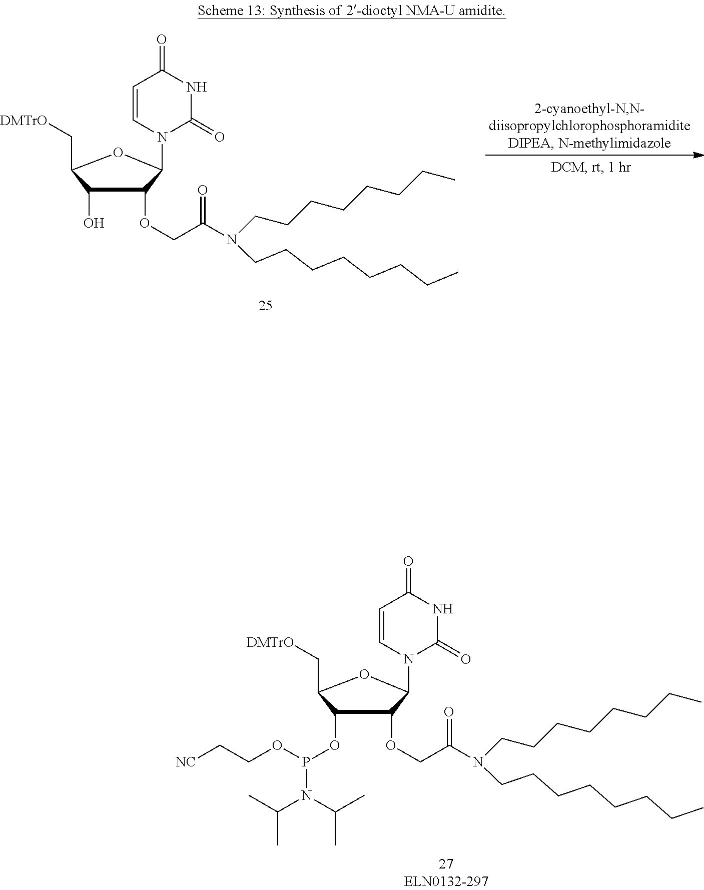

2-[(2R,5R)-5-[[bis(4-methoxyphenyl)-phenyl-methoxy]methyl]-4-[2-cyanoethoxy-(diisopropylamino)phosphanyl]oxy-2-(2,4-dioxopyrimidin-1-yl)tetrahydrofuran-3-yl]oxy-N,N-dioctyl-acetamide 27: To a clear solution of 25 (0.28 g, 338.15 μmol) in DCM (15 mL) at 22° C. was added N-methyl imidazole (56.08 mg, 676.29 mol, 54.45 μL) and DIPEA (220.72 mg, 1.69 mmol, 297.46 μL) The reaction mixture was stirred for 5 minutes at 22° C. and 2-cyanoethyl-N,N-diisopropylchlorophosphoramidite (168.49 mg, 676.29 μmol, 158.95 μL) was added slowly into it. Reaction was kept for stirring at 22° C. and TLC was checked after 1 hr. Reaction mixture was diluted with DCM (20 mL) and washed with 10% NaHCO₃ solution (2×30 mL). Organic layer separated, dried over anhydrous Na₂SO₄, filtered and the filtrate was evaporated to dryness. The crude mass obtained was purified by combiflash chromatography (gradient: 20-60% EtOAc in hexane) to afford 27 (0.29 g, 83% yield) as white foam. ¹H NMR (400 MHz, CD₃CN) δ 9.02 (s, 1H), 7.92-7.51 (m, 1H), 7.47-7.37 (m, 2H), 7.37-7.20 (m, 7H), 6.92-6.83 (m, 4H), 5.97 (dd, J=10.8, 5.0 Hz, 1H), 5.29 (dd, J=8.1, 6.3 Hz, 1H), 4.57-4.46 (m, 1H), 4.42-4.12 (m, 4H), 3.92-3.51 (m, 10H), 3.44-3.02 (m, 5H), 2.68 (dt, J=6.4, 5.2 Hz, 1H), 2.51 (t, J=6.0 Hz, 1H), 1.57-1.42 (m, 4H), 1.33-1.13 (m, 30H), 1.04 (d, J=6.8 Hz, 3H), 0.91-0.82 (m, 6H) ppm. ¹³C NMR (101 MHz, CD₃CN) δ 168.63, 168.57, 163.77, 159.80, 159.78, 151.48, 151.45, 145.70, 145.65, 141.57, 141.39, 136.52, 136.47, 136.43, 136.35, 131.19, 131.12, 131.10, 129.12, 129.06, 128.98, 128.00, 119.62, 119.36, 114.17, 102.73, 102.70, 88.40, 88.01, 87.76, 87.74, 84.18, 83.90, 83.85, 81.96, 81.93, 81.27, 81.22, 71.94, 71.80, 71.69, 71.51, 69.68, 69.65, 69.29, 63.72, 63.38, 60.97, 60.01, 59.83, 59.26, 59.06, 55.95, 55.94, 47.63, 47.55, 46.30, 44.20, 44.07, 43.95, 32.57, 30.11, 30.09, 30.01, 29.99, 29.72, 28.40, 27.72, 27.63, 27.61, 25.17, 25.09, 25.07, 24.99, 24.94, 24.87, 23.37, 21.11, 21.04, 20.96, 14.41 ppm. ³¹P NMR (162 MHz, CD₃CN) δ 151.86, 151.69 ppm. HRMS calc. for C57H₈₃N₅O₁₀P [M+H]⁺ 1028.5878, found 1028.5872.

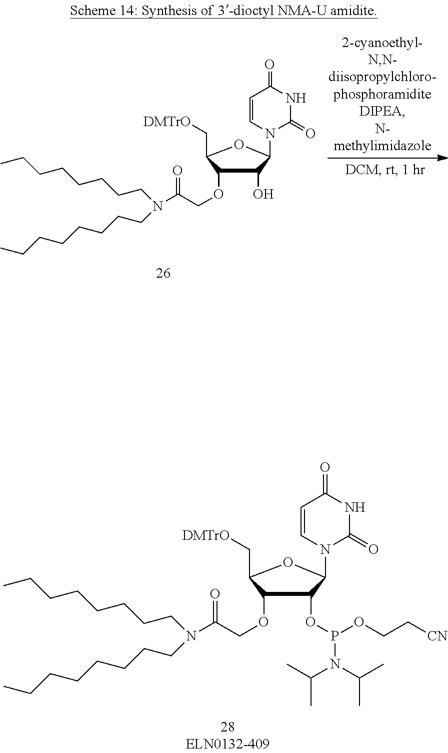

2-[(2R,5R)-2-[[bis(4-methoxyphenyl)-phenyl-methoxy]methyl]-4-[2-cyanoethoxy-(diisopropylamino)phosphanyl]oxy-5-(2,4-dioxopyrimidin-1-yl)tetrahydrofuran-3-yl]oxy-N,N-dioctyl-acetamide 28: To a clear solution of 26 (0.6 g, 724.60 μmol) in DCM (20 mL) at 22° C. was added N-methyl imidazole (120.18 mg, 1.45 mmol, 116.68 μL) and DIPEA (472.97 mg, 3.62 mmol, 637.42 μL). The reaction mixture was stirred for 5 minutes and 2-cyanoethyl-N,N-diisopropylchlorophosphoramidite (361.05 mg, 1.45 mmol, 340.61 μL) was added slowly into it. Reaction was kept for stirring at 22° C. and TLC was checked after 1 hr. Reaction mixture was diluted with dichloromethane (20 mL) and washed with 10% NaHCO₃ solution (2×30 mL). Organic layer separated, dried over anhydrous Na₂SO₄, filtered and the filtrate was evaporated to dryness. The crude mass obtained was purified by combiflash chromatography (gradient: 20-60% EtOAc in hexane) to afford 28 (0.55 g, 74% yield) as white foam. ¹H NMR (600 MHz, CD₃CN) δ 9.09 (s, 1H), 7.82-7.62 (m, 1H), 7.46 (dt, J=6.9, 3.3 Hz, 2H), 7.42-7.16 (m, 7H), 6.89 (ft, J=6.3, 3.0 Hz, 4H), 6.16-5.88 (m, 1H), 5.39-5.21 (m, 1H), 4.57-4.48 (m, 1H), 4.47-4.35 (m, 1H), 4.33-4.17 (m, 3H), 3.92-3.81 (m, 2H), 3.81-3.78 (m, 6H), 3.70-3.60 (m, 2H), 3.54-3.34 (m, 2H), 3.29-3.22 (m, 2H), 3.17-3.06 (m, 2H), 2.69-2.61 (m, 2H), 1.56-1.45 (m, 4H), 1.32-1.22 (m, 21H), 1.22-1.15 (m, 12H), 0.89 (pt, J=5.0, 2.7 Hz, 6H) ppm. ¹³C NMR (151 MHz, CD₃CN) δ 168.64, 168.55, 163.74, 163.69, 159.73, 159.70, 159.67, 151.31, 151.27, 145.78, 145.74, 141.23, 140.95, 136.57, 136.53, 136.41, 136.32, 131.12, 131.07, 129.04, 128.95, 128.91, 127.96, 127.93, 119.51, 119.45, 114.14, 114.10, 114.07, 102.73, 102.67, 89.03, 88.53, 88.50, 87.72, 87.58, 82.98, 82.89, 78.57, 78.13, 78.09, 76.08, 75.99, 75.90, 75.78, 69.85, 69.82, 69.68, 63.56, 59.86, 59.74, 59.53, 59.40, 55.89, 47.57, 47.49, 46.33, 46.26, 44.19, 44.11, 44.07, 43.99, 32.56, 32.52, 30.09, 30.06, 29.98, 29.95, 29.68, 29.62, 28.35, 28.31, 27.71, 27.70, 27.57, 27.51, 25.20, 25.15, 24.93, 24.87, 24.85, 24.81, 23.34, 22.75, 21.30, 21.02, 20.98, 20.86, 20.82, 14.39 ppm. ³¹P NMR (243 MHz, CD₃CN) δ 150.50, 150.23 ppm. HRMS calc. for C57H₈₃N₅O₁₀P [M+H]⁺ 1028.5878.

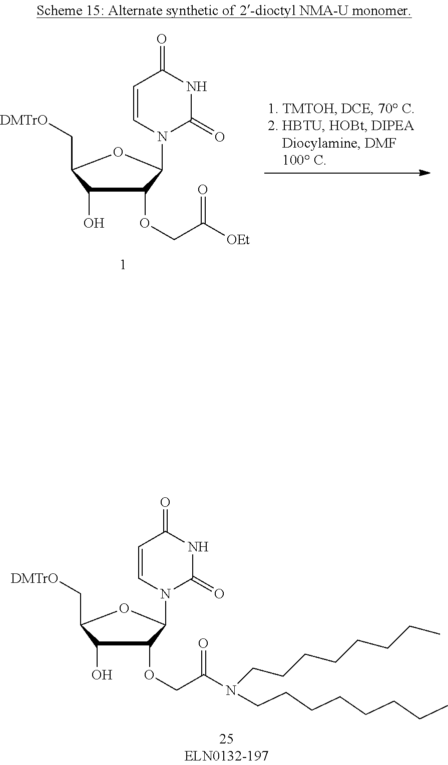

2-[(2R,5R)-5-[[bis(4-methoxyphenyl)-phenyl-methoxy]methyl]-2-(2,4-dioxopyrimidin-1-yl)-4-hydroxy-tetrahydrofuran-3-yl]oxy-N,N-dioctyl-acetamide [25]: To a clear solution of 1 (0.34 g, 537.42 μmol) in dry dichloroethane (30 mL) was added trimethyltin hydroxide (495.79 mg, 2.69 mmol) and stirred at 70° C. for 5 hrs. TLC was checked and all the volatile matters were evaporated to dryness. Crude mass was purified through flash chromatography (gradient: 0-7% MeOH in DCM) and subjected to next step. The dried, pure fraction was dissolved in dry dimethylformamide (15.00 mL) and to the mixture was added HBTU (247.04 mg, 644.90 μmol) and 1-hydroxybenzotriazole hydrate (99.76 mg, 644.90 mol) in single portions. To the resulting mixture DIPEA (210.47 mg, 1.61 mmol, 283.66 μL) was added and stirred for 5 minutes. Finally, dioctylamine (160.53 mg, 644.90 μmol, 200.92 μL) was added and the reaction mixture was stirred for 15 hrs at 22° C. and then heated at 100° C. for 8 hrs. Reaction mixture was diluted with EtOAc (30 mL) and brine (30 mL). Organic layer was separated, and aqueous layer was washed with EtOAc (20 mL). Combined organic layer was washed with NaHCO₃ solution (2×30 mL), NH₄Cl (2×30 mL) and brine (3×30 mL). EtOAc layer was dried over anhydrous Na₂SO₄, filtered and the filtrate was evaporated to dryness. Crude mass was purified by column chromatography (gradient: 20-70% EtOAc in hexane) to afford 25 (0.29 g, 65% yield) as white solid. ¹H NMR (400 MHz, CDCl₃) δ 8.12 (s, 1H), 8.07 (d, J=8.1 Hz, 1H), 7.43-7.36 (m, 2H), 7.34-7.16 (m, 7H), 6.88-6.80 (m, 4H), 6.08 (d, J=4.8 Hz, 1H), 5.83 (d, J=1.0 Hz, 1H), 5.26 (d, J=8.2 Hz, 1H), 4.60 (d, J=15.9 Hz, 1H), 4.52-4.41 (m, 2H), 4.17 (dt, J=8.7, 2.3 Hz, 1H), 3.93-3.87 (m, 1H), 3.79 (d, J=1.6 Hz, 6H), 3.31 (q, J=8.5 Hz, 2H), 3.08 (t, J=7.8 Hz, 2H), 1.54 (t, J=8.5 Hz, 4H), 1.33-1.24 (m, 21H), 0.88 (td, J=6.8, 4.6 Hz, 6H) ppm. ¹³C NMR (101 MHz, CDCl₃) δ 169.67, 162.99, 158.82, 158.76, 150.11, 144.74, 140.18, 135.64, 135.38, 130.39, 130.24, 128.31, 128.12, 127.18, 113.45, 113.41, 101.84, 89.56, 87.00, 86.53, 83.84, 68.87, 67.77, 61.23, 55.39, 46.73, 46.62, 31.93, 31.88, 29.46, 29.42, 29.36, 29.31, 28.83, 27.68, 27.15, 27.01, 22.77, 14.23 ppm. HRMS calc. for C₄₈H₆₅N₃O₉Na [M+Na]⁺ 850.4619, found 850.4608.

### Functionalized NMA Amidites:

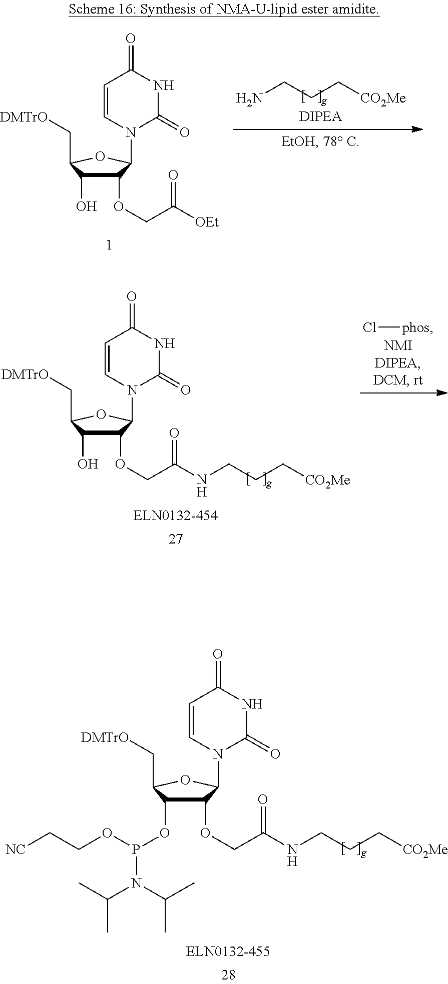

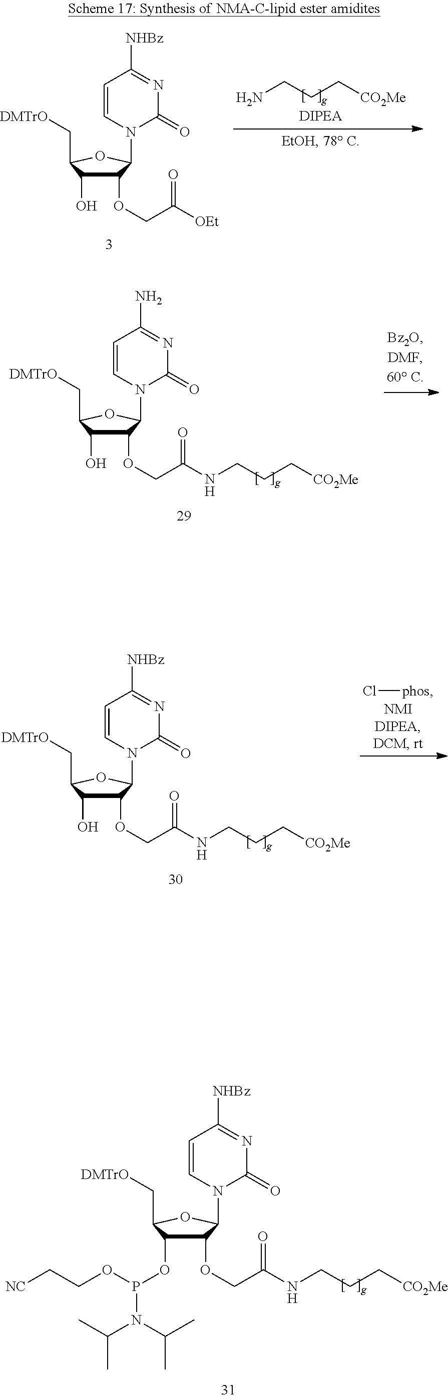

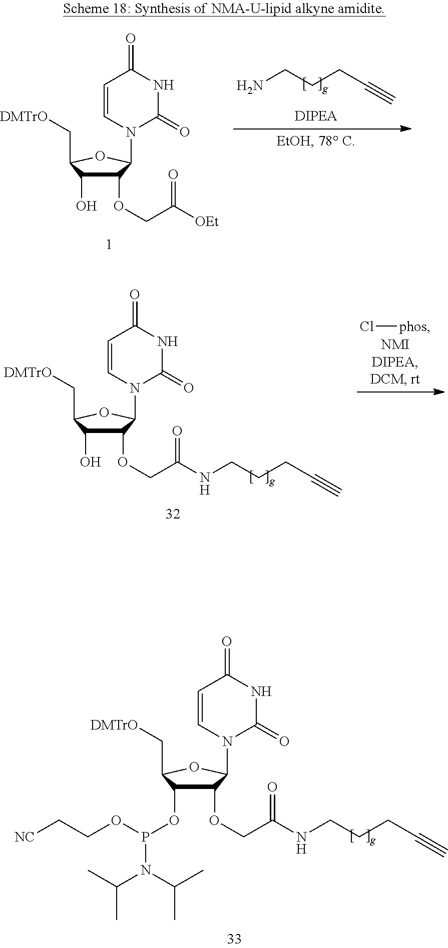

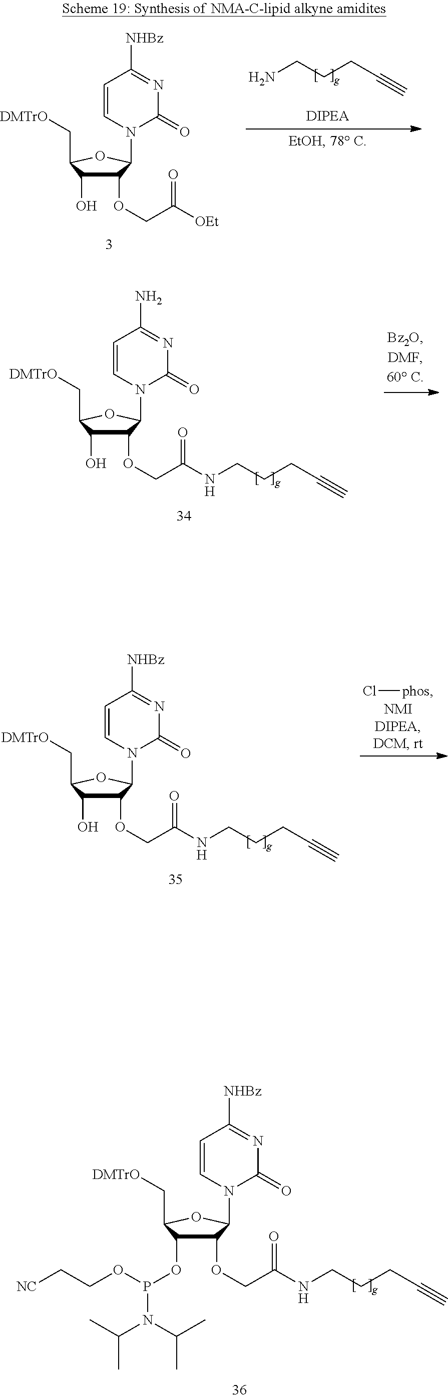

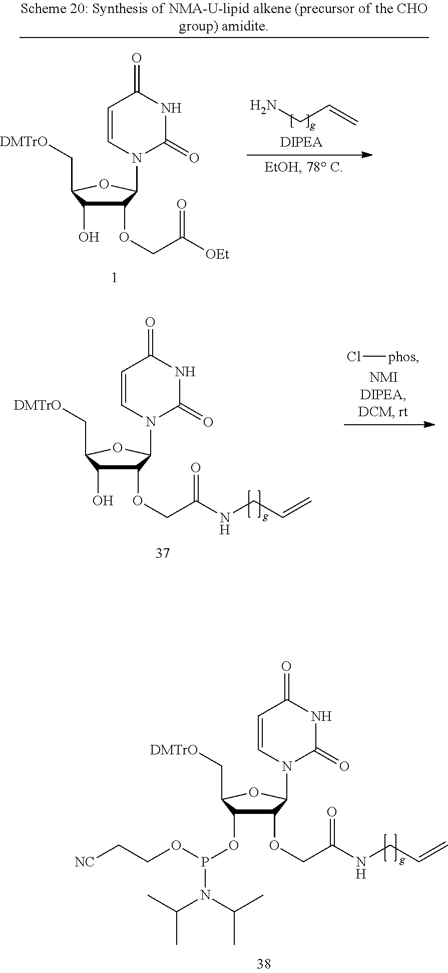

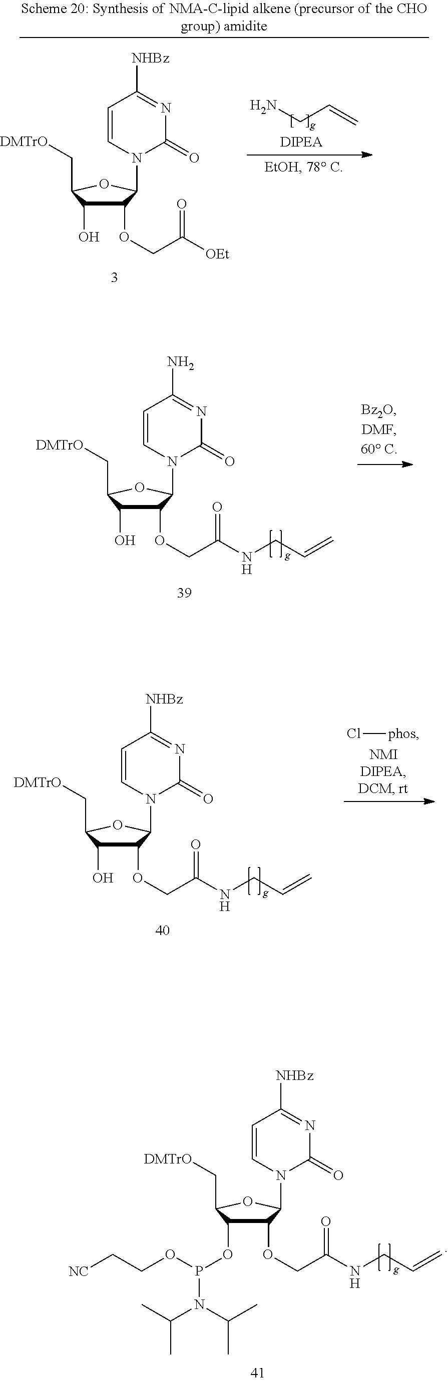

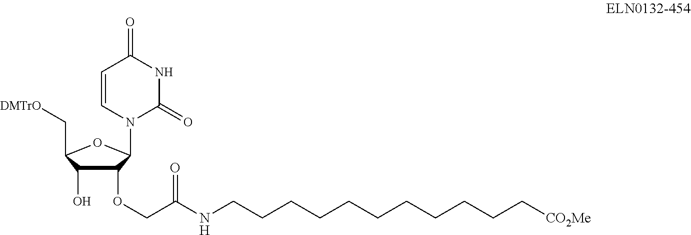

Methyl-12-[[2-[(2R,5R)-5-[[bis(4-methoxyphenyl)-phenyl-methoxy]methyl]-2-(2,4-dioxopyrimidin-1-yl)-4-hydroxy-tetrahydrofuran-3-yl]oxyacetyl]amino]dodecanoate [27]: To a clear solution of compound 1 (3.0 g, 4.74 mmol) in ethanol (50 mL) was added methyl-12-aminododecanoate (1.63 g, 7.11 mmol) and diisopropylethylamine (1.23 g, 9.48 mmol, 1.65 mL) in single portions. The reaction mixture was stirred under refluxing condition for 16 hr. TLC was checked and all the volatile matters were evaporated. The crude residue thus obtained was diluted with EtOAc (20 mL) and washed with saturated NH₄Cl solution (30 mL). Organic layer was then separated, dried over anhydrous Na₂SO₄, filtered and filtrated was evaporated to dryness. Crude material thus obtained, was purified by flash chromatography (gradient: 40-90% EtOAc in hexane followed by 5% MeOH in DCM) to afford 27 (2.5 g, 65% yield) as yellowish-white foam. ¹H NMR (600 MHz, DMSO-d₆) δ 11.43 (d, J=2.2 Hz, 1H), 7.92 (t, J=5.9 Hz, 1H), 7.74 (d, J=8.1 Hz, 1H), 7.41-7.36 (m, 2H), 7.35-7.29 (m, 2H), 7.29-7.22 (m, 5H), 6.93-6.87 (m, 4H), 5.80 (d, J=2.2 Hz, 1H), 5.59 (d, J=7.6 Hz, 1H), 5.25 (dd, J=8.1, 2.2 Hz, 1H), 4.24 (td, J=7.7, 5.1 Hz, 1H), 4.16 (d, J=15.3 Hz, 1H), 4.02 (dt, J=9.4, 6.3 Hz, 3H), 3.74 (s, 7H), 3.56 (s, 3H), 3.33 (dd, J=10.9, 4.6 Hz, 1H), 3.26 (dd, J=10.9, 2.3 Hz, 1H), 3.14-3.04 (m, 2H), 2.27 (t, J=7.4 Hz, 2H), 1.49 (p, J=7.6 Hz, 2H), 1.40 (p, J=6.9 Hz, 2H), 1.22 (d, J=7.1 Hz, 17H) ppm. ¹³C NMR (151 MHz, DMSO-d₆) δ 173.44, 168.72, 163.18, 158.19, 150.29, 144.68, 140.10, 135.41, 135.07, 129.83, 127.99, 127.75, 126.87, 113.31, 113.29, 101.32, 87.70, 85.90, 82.30, 81.88, 69.27, 68.37, 62.21, 55.09, 51.22, 38.22, 33.29, 29.19, 29.02, 29.00, 28.93, 28.80, 28.75, 28.51, 26.47, 24.48 ppm.

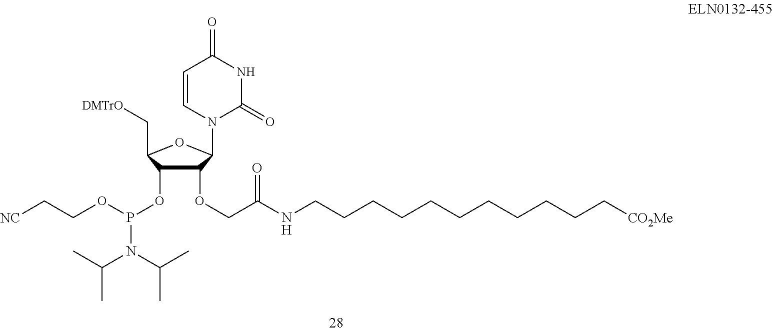

Methyl-12-[[2-[(2R,5R)-4-[2-cyanoethoxy-(diisopropylamino)phosphanyl]oxy-2-(2,4-dioxopyrimidin-1-yl)-5-[[(4-methoxyphenyl)-diphenyl-methoxy]methyl]tetrahydrofuran-3-yl]oxyacetyl]amino]dodecanoate [28]: To a clear solution of To a clear solution of 27 (1.2 g, 1.47 mmol) in dichloromethane (30 mL) was added N-methylimidazole (181.12 mg, 2.21 mmol, 175.84 μL) and diisopropylethylamine (950.36 mg, 7.35 mmol, 1.28 mL) in single portions. After stirring the reaction mixture for 5 minutes at 22° C., 2-cyanoethyl N,N-diisopropylchlorophosphoramidite (696.16 mg, 2.94 mmol, 656.76 μL) was added and continued stirring for 1 hr and TLC was checked. Starting material was consumed and reaction mixture was diluted with DCM (15 mL). DCM layer was washed with 10% NaHCO₃ (2×25 mL) solution, and brine (30 mL). Organic layer was separated, dried over anhydrous Na₂SO₄, filtered and filtrate was evaporated at 36° C. to afford crude compound which was purified by flash chromatography (30-70% EtOAc in hexane) to afford 28 (1.22 g, 84% yield) as white foam. ¹H NMR (600 MHz, CD₃CN) δ 9.41 (s, 1H), 7.87-7.79 (m, 1H), 7.51-7.43 (m, 2H), 7.44-7.15 (m, 8H), 7.02-6.87 (m, 5H), 5.92 (dd, J=8.3, 2.4 Hz, 1H), 5.34-5.16 (m, 1H), 4.53 (tdd, J=10.1, 6.3, 3.0 Hz, 1H), 4.31-4.11 (m, 5H), 3.90-3.73 (m, 7H), 3.71-3.56 (m, 5H), 3.52 (dt, J=11.3, 2.4 Hz, 1H), 3.49-3.39 (m, 1H), 3.27-3.15 (m, 2H), 2.72-2.67 (m, 1H), 2.55 (t, J=6.0 Hz, 1H), 2.29 (t, J=7.5 Hz, 2H), 1.56 (p, J=7.2 Hz, 2H), 1.48 (h, J=7.2 Hz, 2H), 1.28 (d, J=6.9 Hz, 17H), 1.22-1.12 (m, 11H), 1.05 (d, J=6.8 Hz, 3H) ppm. ³¹P NMR (243 MHz, CD₃CN) δ 150.30, 148.28 ppm. ¹³C NMR (151 MHz, CD₃CN) δ 174.79, 169.59, 169.35, 164.05, 164.03, 159.73, 159.71, 159.70, 151.40, 151.38, 145.71, 145.63, 140.86, 140.78, 136.37, 136.29, 136.26, 136.15, 131.16, 131.13, 131.09, 129.02, 128.92, 127.98, 119.59, 119.40, 114.08, 102.48, 102.43, 89.42, 89.03, 87.58, 83.21, 83.19, 83.06, 82.73, 82.69, 82.33, 82.30, 71.53, 71.43, 70.81, 70.74, 70.64, 70.48, 70.46, 62.24, 59.42, 59.34, 59.29, 59.20, 55.89, 55.87, 51.83, 44.05, 44.00, 43.97, 43.92, 39.45, 39.36, 34.44, 30.46, 30.41, 30.24, 30.23, 30.22, 30.14, 30.02, 30.01, 29.94, 29.73, 27.62, 27.60, 25.63, 25.08, 25.03, 25.00, 24.95, 24.90, 24.85, 24.76, 24.72, 21.05, 21.03, 21.01, 20.98 ppm.

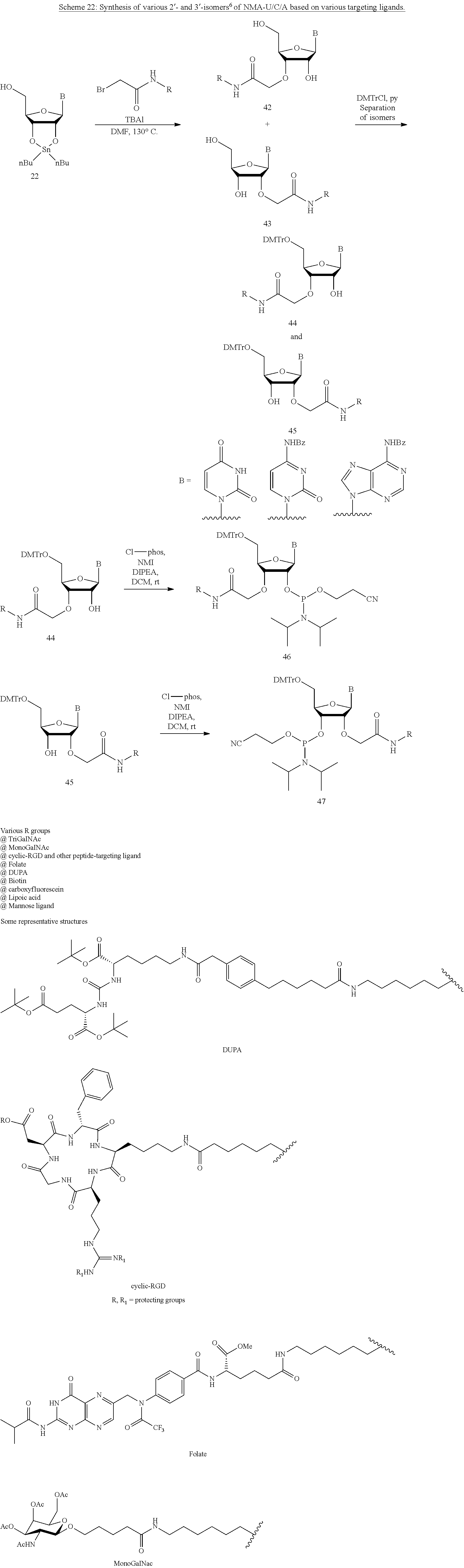

Extending the scope of NMA to NMCE

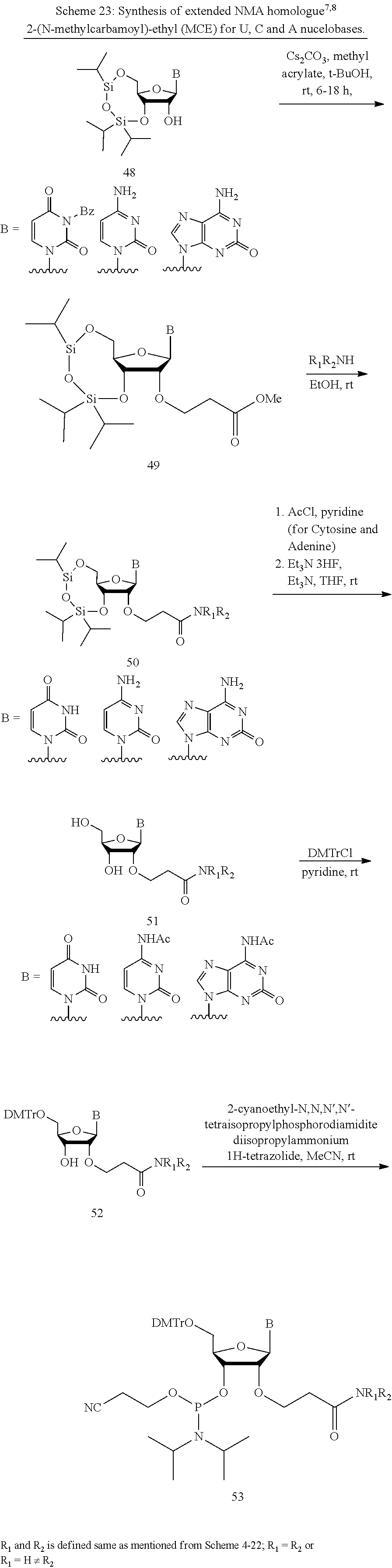

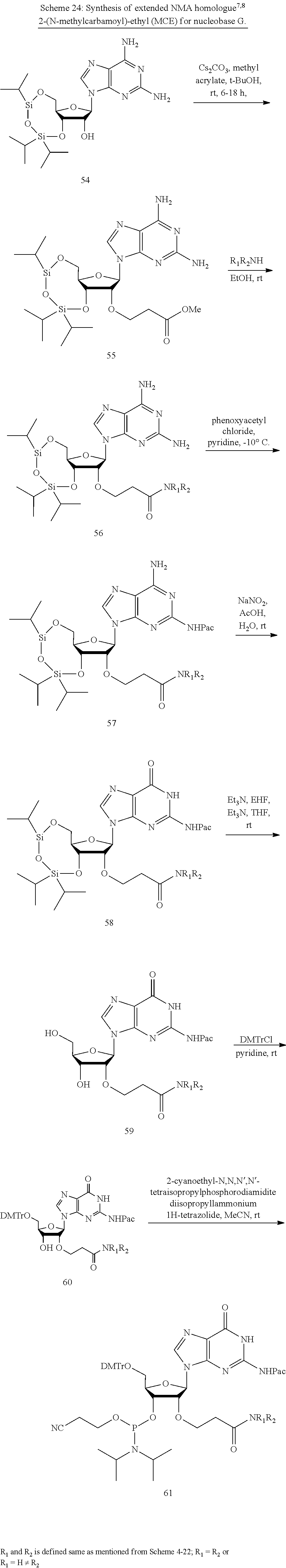

### NMA Chemistry at 5′-End:

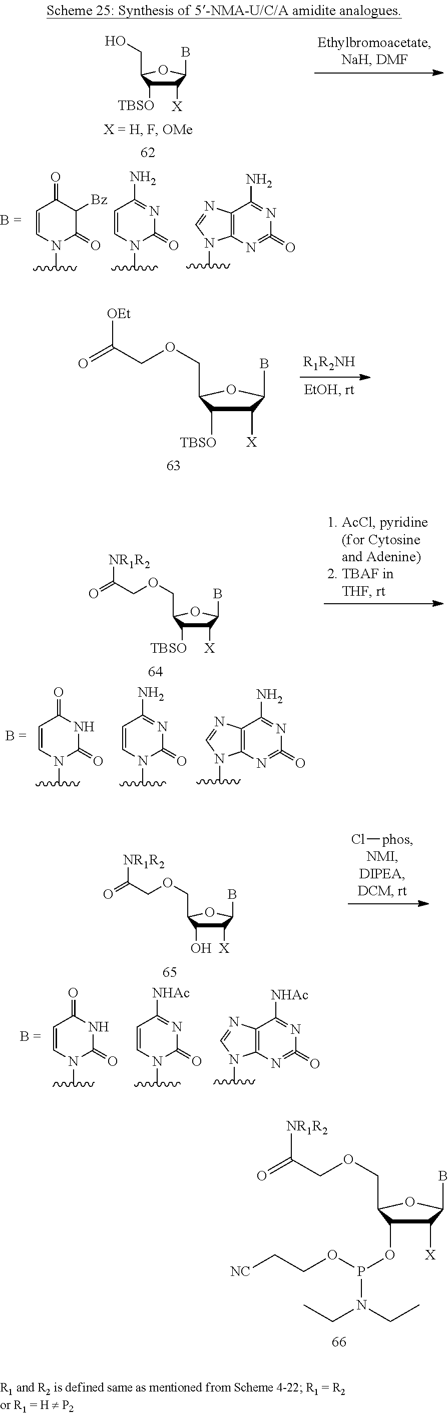

### REFERENCES

(1) Prakash, T. P.; Kawasaki, A. M.; Lesnik, E. A.; Owens, S. R.; Manoharan, M. Organic Letters 2003, 5, 403.
(2) Ozaki, H.; Momiyama, S.; Yokotsuka, K.; Sawai, H. Tetrahedron Letters 2001, 42, 677.
(3) Kachalova, A. V.; Zatsepin, T. S.; Romanova, E. A.; Stetsenko, D. A.; Gait, M. J.; Oretskaya, T. S. Nucleosides, Nucleotides & Nucleic Acids 2000, 19, 1693.
(4) Keller, T. H.; Haener, R. Nucleic Acids Res. 1993, 21, 4499.
(5) Barde, E.; Guérinot, A.; Cossy, J. Organic Letters 2017, 19, 6068.
(6) Wagner, D.; Verheyden, J. P. H.; Moffatt, J. G. J. Org. Chem. 1974, 39, 24.
(7) Yamada, T.; Okaniwa, N.; Saneyoshi, H.; Ohkubo, A.; Seio, K.; Nagata, T.; Aoki, Y.; Takeda, S. i.; Sekine, M. The Journal of Organic Chemistry 2011, 76, 3042.
(8) Yamada, T.; Masaki, Y.; Okaniwa, N.; Kanamori, T.; Ohkubo, A.; Tsunoda, H.; Seio, K.; Sekine, M. Organic & Biomolecular Chemistry 2014, 12, 6457.

### Example 2: MOE and MTE Lipophilic Phosphoramidites

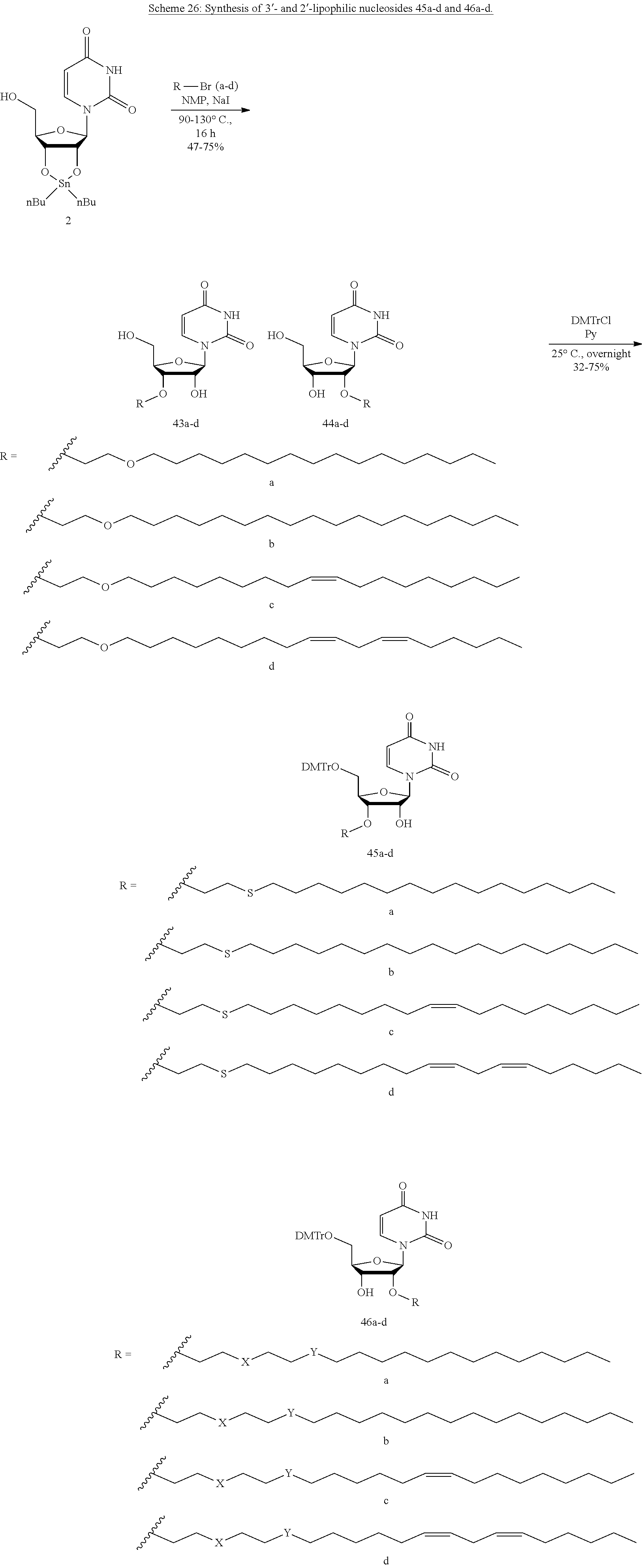

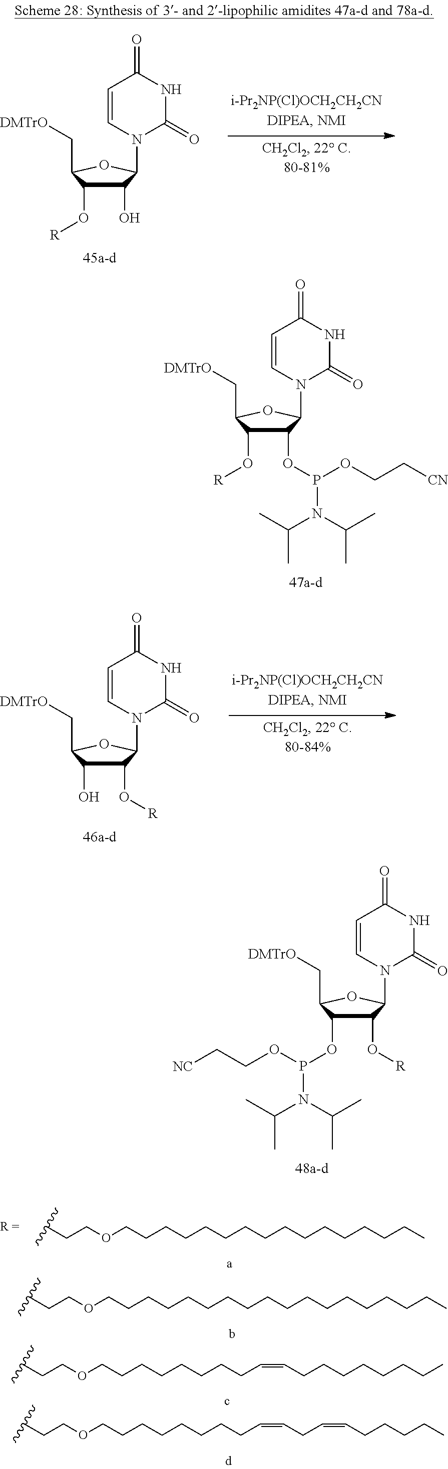

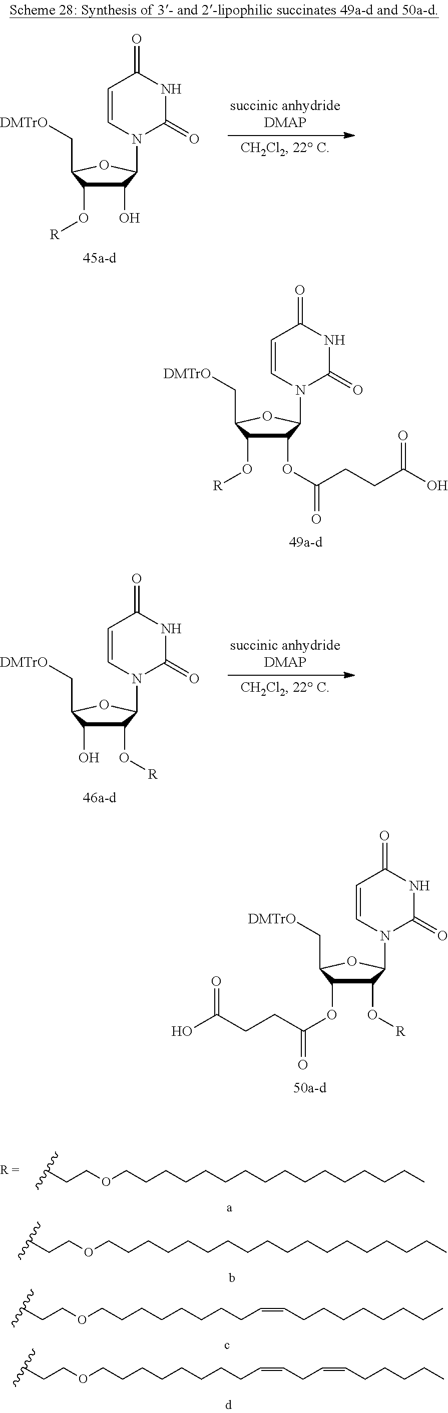

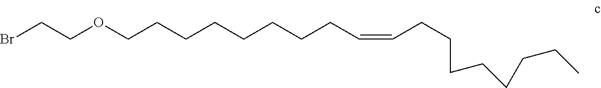 [SMILES: CCCCCCCC/C=C\CCCCCCCCOCCBr]

Synthesis of (9Z)-1-(2-bromoethoxy)octadec-9-ene: To a solution of ethylene glycol (138.23 g, 2.23 mol) in DMF (DMF) (2.70 L) under nitrogen atmosphere was added sodium hydride (NaH) (26.72 g, 0.67 mol) in portions over 30 min at 10° C. The mixture was stirred for additional 30 min at 10° C. To this mixture was added (9Z)-1-bromooctadec-9-ene² (184.50 g, 0.44 mol). The resulting mixture was stirred overnight at 50° C. under nitrogen atmosphere. The reaction was quenched by the addition of saturated NH₄Cl solution (9 L) at 0° C. The resulting mixture was extracted with DCM (3×3 L). The combined organic layers were dried over anhydrous Na₂SO₄. After filtration, the filtrate was concentrated under reduced pressure. The residue was purified by silica gel column chromatography, eluted with PE/EtOAc (12:1) to afford (Z)-2-(octadec-9-en-1-yloxy)ethan-1-ol (112 g, 64%) as a colorless oil. ¹H NMR, (400 MHz, CDCl₃) δ 5.43-5.33 (m, 2H), 3.78-3.71 (m, 2H), 3.59-3.53 (m, 2H), 3.49 (t, J=6.7 Hz, 2H), 2.08-1.95 (m, 3H), 1.99 (s, 1H), 1.62 (q, J=6.9 Hz, 2H), 1.42-1.26 (m, 23H), 0.95-0.86 (m, 3H) ppm.

To a stirred solution of (Z)-2-(octadec-9-en-1-yloxy)ethan-1-ol (112.00 g, 286.68 mmol,) in DCM (1.68 L) were added CBr₄ (123.59 g, 0.37 mol) and PPh₃ (97.75 g, 0.373 mol) in portions at 10° C. under nitrogen atmosphere. The resulting mixture was stirred for 30 minutes at 10° C. under nitrogen atmosphere. The reaction was quenched by the addition of water (2 L) at room temperature. The aqueous layer was extracted with DCM (1×1 L). The combined organic layers were dried over anhydrous Na₂SO₄. After filtration, the filtrate was concentrated under reduced pressure. The residue was purified by silica gel column chromatography, eluted with PE/EA (100:1) to afford (9Z)-1-(2-bromoethoxy)octadec-9-ene (123 g, 91%) as a colorless oil. ¹H NMR (300 MHz, CDCl₃) δ 5.45-5.30 (m, 2H), 3.75 (t, J=6.3 Hz, 2H), 3.50 (dq, J=12.6, 6.5, 5.6 Hz, 4H), 2.09-1.92 (m, 3H), 1.68-1.19 (m, 23H), 0.95-0.84 (m, 3H) ppm. ¹³C NMR (101 MHz, CDCl₃) δ 130.55, 130.42, 130.08, 129.96, 71.49, 70.75, 32.76, 32.73, 32.08, 32.06, 30.61, 29.92, 29.89, 29.85, 29.83, 29.81, 29.76, 29.74, 29.74, 29.68, 29.64, 29.62, 29.59, 29.55, 29.51, 29.47, 29.39, 29.33, 29.23, 27.36, 27.34, 26.20, 22.83, 14.27 ppm. HRMS calc. for C₂₀H₄₀BrO [M+H]⁺ 375.2263, found 375.2270.

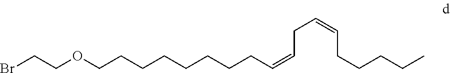 [SMILES: CCCCC/C=C\C/C=C\CCCCCCCCOCCBr]

Synthesis of (6Z,9Z)-18-(2-bromoethoxy)octadeca-6,9-diene 2d: To a clear solution of ethylene glycol (94.22 g, 1.52 mol) in DMF (1.00 L) under inert atmosphere of nitrogen was added NaH (14.57 g, 0.61 mol), in portions at 0-10° C. in 2 h. To this resulting solution, (6Z,9Z)-18-bromooctadeca-6,9-diene² (100.00 g, 0.30 mol), dissolved in DMF (200 mL) was added at 25° C. The resulting solution was stirred for 16 h at 30° C. The resulting solution was diluted with 1 L of water. The resulting solution was extracted with DCM (3×500 mL) and the organic layers combined. The resulting mixture was washed with water (3×1 L). The organic layer was separated, dried over anhydrous Na₂SO₄, filtered and the filtrate was concentrated under vacuum. The residue thus obtained was purified by column chromatography to afford (Gradient: 20% EA in PE) 2-[(9Z,12Z)-octadeca-9,12-dien-1-yloxy]ethanol (60 g, 57%) as colorless oil. ¹H NMR (400 MHz, CDCl₃) δ 5.43-5.26 (m, 4H), 3.75-3.69 (m, 2H), 3.56-3.49 (m, 2H), 3.46 (t, J=6.7 Hz, 2H), 2.77 (t, J=6.7 Hz, 2H), 2.04 (q, J=6.7 Hz, 5H), 1.59 (q, J=6.9 Hz, 2H), 1.41-1.31 (m, 2H), 1.35-1.22 (m, 15H), 0.88 (t, J=6.8 Hz, 3H) ppm. ¹³C NMR (101 MHz, CDCl₃) δ 130.35, 130.26, 128.13, 128.06, 71.82, 71.56, 62.03, 31.68, 29.81, 29.63, 29.60, 29.50, 29.40, 27.37, 27.35, 26.26, 25.78, 22.73, 14.23 ppm. HRMS calc. for C₂₀H₃₈O₂Na [M+Na]⁺ 333.2770, found 333.2778.

2-[(9Z,12Z)-Octadeca-9,12-dien-1-yloxy]ethanol (60.00 g, 193.223 mmol, 1.00 equiv) was dissolved in DCM (600.00 mL) under inert atmosphere. To this resulting solution was added CBr₄ (83.30 g, 0.25 mol) at 0° C. To this mixture was added PPh₃ (65.88 g, 0.25 mmol) in portions at 0-5° C. through 0.5 h. The resulting solution was stirred for 5 h at 25° C. The resulting mixture was concentrated under vacuum. The crude residue was purified by column chromatography (gradient: 15% EtOAc in hexane) to afford d (40 g, 50%) as a colorless solid. ¹H NMR (400 MHz, CDCl₃) δ 5.67-5.04 (m, 4H), 3.73 (t, J=6.3 Hz, 2H), 3.47 (dt, J=8.9, 6.5 Hz, 4H), 2.96-2.48 (m, 2H), 2.05 (q, J=6.8 Hz, 4H), 1.68-1.47 (m, 2H), 1.40-1.18 (m, 17H), 0.97-0.68 (m, 3H) ppm. ¹³C NMR (101 MHz, CDCl₃) δ 130.30, 130.23, 128.11, 128.05, 71.46, 70.75, 31.67, 30.59, 29.79, 29.75, 29.61, 29.54, 29.49, 29.38, 27.36, 27.34, 26.19, 25.77, 22.71, 14.21 ppm.

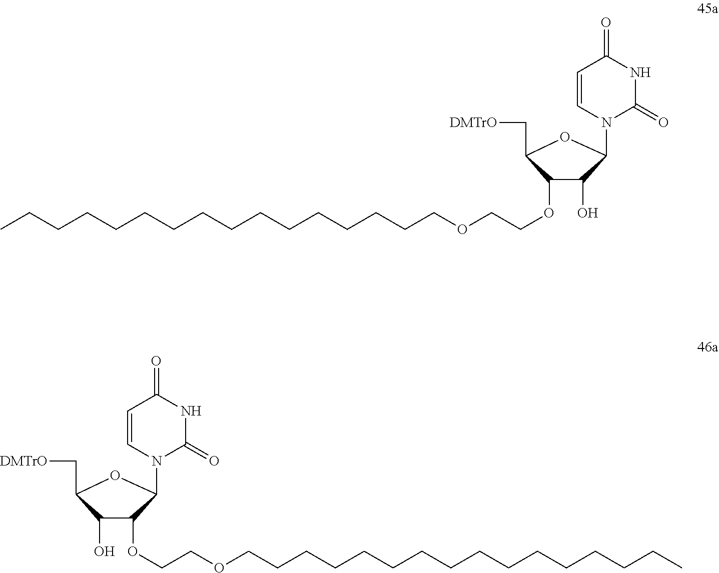

Synthesis of compound 1-(5-[[bis(4-methoxyphenyl)(phenyl)methoxy]methyl]-4-[2-(hexadecyloxy)ethoxy]-3-hydroxyoxolan-2-yl)-3H-pyrimidine-2,4-dione 45a and 1-(5-[[bis(4-methoxyphenyl)(phenyl)methoxy]methyl]-3-[2-(hexadecyloxy)ethoxy]-4-hydroxyoxolan-2-yl)-3H-pyrimidine-2,4-dione 46a: To a solution of 23 (91.0 g, 191.53 mmol) in N-methyl-2-pyrrolidone (NMP) (1.80 L) was added 1-(2-bromoethoxy)hexadecane (a) (133.84 g, 383.06 mmol)⁴ and sodium iodide (NaI) (57.42 g, 383.05 mmol) under nitrogen atmosphere. The resulting mixture was stirred for additional 16 h at 130° C. The mixture was cooled to room temperature (rt) and extracted with DCM (DCM) (3×1.5 L). The combined organic layers were washed with brine (3×1 L), dried over anhydrous MgSO₄. After filtration, the filtrate was concentrated under reduced pressure. The residue was purified by silica gel column chromatography, eluted with DCM: methanol (MeOH) (20:1) to afford the mixture of 43a and 44a (76 g, 75.08%) as a brown solid. To a mixture of 43a and 44a (76.0 g, 143.79 mmol) in pyridine (1.3 L) under nitrogen atmosphere, was added 1-[chloro(4-methoxyphenyl)phenylmethyl]-4-methoxybenzene (DMTrCl) (53.59 g, 158.17) at room temperature. The resulting mixture was stirred for additional overnight at room temperature. The reaction was quenched by the addition of MeOH (1 mL) and concentrated in vacuo. The product residue was dissolved in 3% triethylamine (TEA)/DCM (500 mL) and washed with saturated NaHCO₃ solution (1×150 mL) and brine (1×150 mL). The organic layer was dried over anhydrous Na₂SO₄, filtered and the filtrate was concentrated in vacuo. The residue was purified by silica gel column chromatography, eluted with 3% TEA/pet ether (PE) in ethyl acetate (EA) (1:1) to afford the mixture of 45a and 46a (85.8 g, 75%) as a light yellow solid. The crude product (29 g) was purified by Prep Achiral SFC to afford 45a (11.7 g, 75%) as an off-white solid and 46a (14.50 g, 75%) as a yellow solid.

Compound 45a: ¹H NMR, (300 MHz, DMSO-d₆) δ 11.35 (s, 1H), 7.74 (d, J=8.1 Hz, 1H), 7.42-7.18 (m, 9H), 6.98-6.83 (m, 4H), 5.71 (d, J=3.7 Hz, 1H), 5.37 (d, J=5.5 Hz, 1H), 5.29 (d, J=8.1 Hz, 1H), 4.25 (q, J=4.5 Hz, 1H), 4.09-3.94 (m, 2H), 3.74-3.52 (m, 6H), 3.47 (t, J=4.8 Hz, 2H), 3.31 (s, 3H), 3.37-3.23 (m, 3H), 1.46-1.36 (m, 2H), 1.21 (d, J=5.9 Hz, 26H), 0.89-0.78 (m, 3H) ppm. ¹³C NMR, (75 MHz, DMSO-d₆) δ 163.44, 158.63, 150.91, 145.07, 140.79, 135.79, 135.60, 130.18, 128.27, 128.17, 127.17, 113.64, 101.87, 89.61, 86.46, 81.10, 77.77, 72.80, 70.87, 70.08, 69.65, 62.94, 55.43, 31.78, 29.66, 29.53, 29.51, 29.36, 29.19, 26.08, 22.56, 14.30 ppm. HRMS calc. for C₄₈H₆₆N₂O₉Na [M+Na]⁺ 837.4666, found 837.4659.

Compound 46a: ¹H NMR, (300 MHz, DMSO-d₆) δ 11.37 (s, 1H), 7.71 (d, J=8.1 Hz, 1H), 7.37 (d, J=7.2 Hz, 2H), 7.36-7.21 (m, 5H), 7.23 (d, J=1.4 Hz, 2H), 6.89 (d, J=8.7 Hz, 4H), 5.84-5.71 (m, 1H), 5.29 (d, J=8.0 Hz, 1H), 5.07 (d, J=6.5 Hz, 1H), 4.19 (q, J=6.2 Hz, 1H), 3.97 (dt, J=11.5, 3.5 Hz, 2H), 3.77-3.62 (m, 6H), 3.50 (dd, J=6.0, 3.9 Hz, 2H), 3.43-3.17 (m, 6H), 1.42 (q, J=7.1 Hz, 2H), 1.21 (d, J=3.0 Hz, 26H), 0.96-0.79 (m, 3H) ppm. ¹³C NMR, (75 MHz, DMSO-d₆) δ 162.90, 158.14, 150.27, 144.59, 140.15, 135.32, 135.08, 131.34, 129.73, 128.57, 127.77, 126.70, 113.15, 101.46, 87.21, 85.91, 82.57, 81.28, 70.37, 69.39, 69.27, 68.56, 64.91, 62.62, 54.95, 54.78, 48.43, 31.28, 30.03, 29.99, 29.18, 29.04, 28.87, 28.69, 25.59, 22.06, 18.61, 17.17, 13.81, 13.43 ppm. HRMS calc. for C₄₈H₆₆N₂O₉Na [M+Na]⁺ 837.4666, found 837.4673.

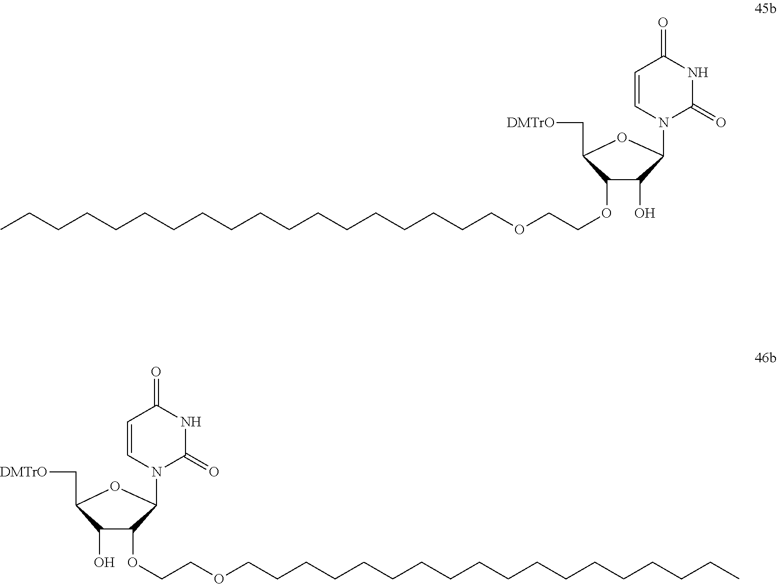

Synthesis of compound 1-(5-[[bis(4-methoxyphenyl)(phenyl)methoxy]methyl]-3-hydroxy-4-[2-(octadecyloxy)ethoxy]oxolan-2-yl)-3H-pyrimidine-2,4-dione 45b and 1-(5-[[bis(4-methoxyphenyl)(phenyl)methoxy]methyl]-4-hydroxy-3-[2-(octadecyloxy)ethoxy]oxolan-2-yl)-3H-pyrimidine-2,4-dione 46b: To a mixture of 2 (41.50 g, 87.34 mmol) in NMP (830.00 mL) was added 1-(2-bromoethoxy)octadecane b⁴ (66.60 g, 174.68 mmol) and NaI (57.42 g, 383.05 mmol) under nitrogen atmosphere. The resulting mixture was stirred for additional 16 h at 130° C. The mixture was cooled to rt and extracted with DCM (3×1 L). The combined organic layers were washed with brine (3×0.7 L), dried over anhydrous MgSO₄. After filtration, the filtrate was concentrated under reduced pressure. The residue was purified by silica gel column chromatography, eluted with DCM/MeOH (20:1) to afford the mixture of 43b and 44b (36.45 g, 74.86%) as off-white solid. To a mixture of 43b and 44b (19.45 g, 34.89 mmol) in pyridine (350 mL) under nitrogen atmosphere was added DMTrCl (13.00 g, 38.37 mmol) at room temperature. The resulting mixture was stirred for additional overnight at room temperature. The reaction was quenched by the addition of MeOH (1 mL) and concentrated in vacuo. The product residue was dissolved in 3% TEA/DCM (150 mL) and washed with saturated NaHCO₃ solution (1×50 mL) and brine (1×50 mL). The organic layer was dried over anhydrous Na₂SO₄, filtered and the filtrate was concentrated in vacuo. The residue was purified by silica gel column chromatography, eluted with 1% TEA/PE in EA (1:1) to afford the mixture of 45b and 46b (14.1 g, 46.4%) as a light yellow solid. The crude product (14.1 g) was purified by achiral SFC to afford 45b (5.25 g, 46.4%) as a light yellow solid and 46b (6.82 g, 46.4%) as a light-yellow solid.

Compound 45b: ¹H NMR, (300 MHz, DMSO-d₆) δ 11.38 (d, J=2.2 Hz, 1H), 7.76 (d, J=8.1 Hz, 1H), 7.43-7.19 (m, 9H), 6.94-6.85 (m, 4H), 5.79-5.69 (m, 1H), 5.40 (d, J=5.4 Hz, 1H), 5.30 (dd, J=8.0, 2.1 Hz, 1H), 4.26 (q, J=4.4 Hz, 1H), 4.11-3.97 (m, 2H), 3.77-3.54 (m, 7H), 3.48 (t, J=4.9 Hz, 2H), 3.38-3.24 (m, 5H), 1.42 (t, J=6.7 Hz, 2H), 1.22 (d, J=5.9 Hz, 30H), 0.90-0.79 (m, 3H) ppm. ¹³C NMR, (75 MHz, CDCl₃) δ 163.46, 158.70, 158.69, 158.61, 150.43, 144.40, 140.53, 139.56, 135.43, 135.27, 130.12, 129.17, 128.14, 128.00, 127.81, 127.12, 127.03, 113.29, 113.15, 102.24, 89.92, 87.00, 81.55, 78.16, 76.68, 74.15, 71.78, 70.21, 69.79, 62.28, 55.24, 31.94, 29.72, 29.69, 29.67, 29.65, 29.60, 29.43, 29.37, 25.97, 22.70, 14.14 ppm. HRMS calc. for C₅₀H₇₀N₂O₀₉ [M+Na]⁺ 865.4979, found 865.4963.

Compound 46b: ¹H NMR, (300 MHz, DMSO-d₆) δ 11.39 (d, J=2.2 Hz, 1H), 7.73 (d, J=8.1 Hz, 1H), 7.39 (d, J=7.2 Hz, 2H), 7.30 (dd, J=22.9, 7.6 Hz, 7H), 6.90 (d, J=8.5 Hz, 4H), 5.86-5.73 (m, 1H), 5.30 (dd, J=8.0, 2.1 Hz, 1H), 5.09 (d, J=6.5 Hz, 1H), 4.20 (q, J=5.9 Hz, 1H), 4.05-3.91 (m, 2H), 3.79-3.65 (m, 7H), 3.56-3.46 (m, 2H), 3.42-3.19 (m, 5H), 1.50-1.39 (m, 2H), 1.22 (d, J=3.7 Hz, 30H), 0.85 (t, J=6.4 Hz, 3H) ppm. ¹³C NMR, (75 MHz, CDCl₃) δ 163.77, 158.72, 158.68, 158.59, 150.45, 144.43, 140.15, 139.57, 135.40, 135.15, 130.21, 130.13, 129.17, 128.19, 128.00, 127.82, 127.13, 127.01, 113.29, 113.12, 102.10, 87.93, 87.02, 83.43, 83.11, 71.65, 71.57, 70.50, 69.58, 68.84, 61.65, 55.23, 31.94, 29.72, 29.68, 29.65, 29.62, 29.53, 29.47, 29.38, 26.04, 22.71, 14.15 ppm. HRMS calc. for C₅₀H₇₀N₂O₉ [M+Na]⁺ 865.4979, found 865.4971.

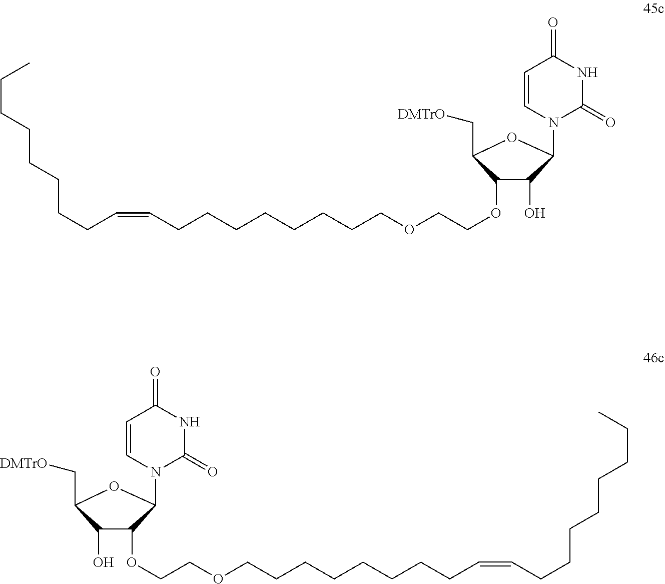

Synthesis of compound 1-(5-[[bis(4-methoxyphenyl)(phenyl)methoxy]methyl]-3-hydroxy-4-[2-[(9Z)-octadec-9-en-1-yloxy]ethoxy]oxolan-2-yl)-3H-pyrimidine-2,4-dione 45c and 1-(5-[[bis(4-methoxyphenyl)(phenyl)methoxy]methyl]-4-hydroxy-3-[2-[(9Z)-octadec-9-en-1-yloxy]ethoxy]oxolan-2-yl)-3H-pyrimidine-2,4-dione 46c: To a mixture of 2 (60.00 g, 126.28 mmol) in NMP (1.2 L) was added (9Z)-1-(2-bromoethoxy) octadec-9-ene c (118.53 g, 252.57 mmol) under nitrogen atmosphere. To this mixture was added NaI (37.86 g, 252.578 mmol) and stirred overnight at 130° C. The mixture was cooled to rt and the reaction was quenched with water (1.2 L). The resulting mixture was extracted with DCM (3×1 L) and the combined organic layers were washed with brine (3×1 L), dried over anhydrous Na₂SO₄. After filtration, the filtrate was concentrated under reduced pressure. The residue was purified by silica gel column chromatography, eluted with DCM/MeOH (40:1) to afford the mixture (57.9 g, 68.09%) of 43c and 44c as off-white solid. To the mixture of 43c and 44c (57.9 g, 85.98 mmol) in pyridine (1.16 L) under nitrogen atmosphere was added DMTrCl (32.05 g, 94.59 mmol) at room temperature. The resulting mixture was stirred overnight at room temperature. The reaction was quenched by the addition of MeOH (1 mL) and concentrated in vacuo. The product residue was dissolved in 3% TEA/DCM (500 mL) and washed with saturated NaHCO₃ solution (1×500 mL) and brine (1×500 mL). The organic layer was dried over anhydrous Na₂SO₄, filtered and the filtrate was concentrated in vacuo. The residue was purified by silica gel column chromatography, eluted with 3% TEA/PE in EA (1:1) to afford the mixture of 45c and 46c (50 g, 80%) as a yellow solid. The crude product (41 g, 80%) was purified by achiral SFC and Prep HPLC twice to afford 45c (6.15 g, 8.50%) and 46c (6.52 g, 9.02%) as off-white solid.

Compound 45c: ¹H NMR, (300 MHz, DMSO-d₆) δ 11.36 (s, 1H), 7.74 (d, J=8.1 Hz, 1H), 7.42-7.32 (m, 2H), 7.36-7.23 (m, 4H), 7.24 (d, J=4.4 Hz, 1H), 7.23 (s, 2H), 6.94-6.83 (m, 4H), 5.72 (d, J=3.7 Hz, 1H), 5.37 (d, J=5.4 Hz, 1H), 5.38-5.22 (m, 3H), 4.25 (q, J=4.2 Hz, 1H), 4.09-3.96 (m, 2H), 3.78-3.65 (m, 7H), 3.59 (dt, J=10.8, 4.8 Hz, 1H), 3.46 (t, J=4.9 Hz, 2H), 3.30 (q, J=6.2 Hz, 4H), 1.96 (q, J=6.6, 6.0 Hz, 4H), 1.50-1.36 (m, 2H), 1.33-1.16 (m, 22H), 0.91-0.77 (m, 3H) ppm. ¹³C NMR, (75 MHz, DMSO-d₆) δ 162.97, 158.13, 158.12, 150.42, 144.59, 140.30, 135.28, 135.07, 129.69, 129.51, 129.48, 127.79, 127.66, 126.68, 113.14, 101.35, 89.10, 85.95, 80.59, 77.22, 72.28, 70.37, 69.59, 69.13, 62.40, 54.93, 31.28, 29.18, 29.11, 28.89, 28.86, 28.70, 28.61, 27.94, 26.59, 26.56, 25.59, 22.08, 13.83 ppm. HRMS calc. for C₅₀H₆₈N₂O₉Na [M+Na]⁺ 863.4823, found 863.4835.

Compound 46c: ¹H NMR, (300 MHz, DMSO-d₆) δ 11.38 (d, J=2.1 Hz, 1H), 7.72 (d, J=8.1 Hz, 1H), 7.42-7.35 (m, 2H), 7.32 (t, J=7.5 Hz, 2H), 7.29-7.18 (m, 5H), 6.94-6.86 (m, 4H), 5.81 (d, J=3.7 Hz, 1H), 5.34-5.26 (m, 3H), 5.08 (d, J=6.6 Hz, 1H), 4.19 (q, J=6.1 Hz, 1H), 3.98 (ddd, J=19.5, 6.0, 3.7 Hz, 2H), 3.74 (s, 6H), 3.72 (t, J=4.2 Hz, 2H), 3.56-3.45 (m, 2H), 3.36 (t, J=6.6 Hz, 2H), 3.33-3.26 (m, 1H), 3.23 (dd, J=10.7, 2.9 Hz, 1H), 2.01-1.92 (m, 4H), 1.44 (p, J=7.2 Hz, 2H), 1.32-1.20 (m, 22H), 0.88-0.80 (m, 3H) ppm. ¹³C NMR, (75 MHz, DMSO-d₆) δ 162.94, 158.13, 150.28, 144.61, 140.17, 135.32, 135.05, 129.74, 129.53, 129.48, 127.80, 127.70, 126.71, 113.16, 101.46, 87.19, 85.90, 82.55, 81.28, 70.38, 69.38, 69.27, 68.54, 62.59, 54.95, 54.82, 31.28, 29.19, 29.12, 29.10, 28.89, 28.85, 28.70, 28.61, 26.59, 26.56, 25.60, 22.08, 13.84 ppm. HRMS calc. for C₅₀H₆₈N₂O₉Na [M+Na]⁺ 863.4823, found 863.4828.

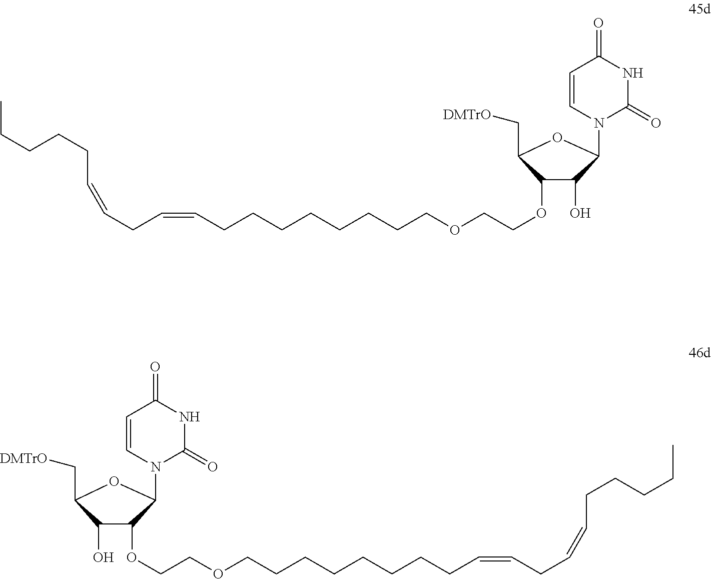

Synthesis of compound 1-[(2R,3R,4S,5R)-5-[[bis(4-methoxyphenyl)(phenyl)methoxy]methyl]-3-hydroxy-4-[2-[(9Z,12Z)-octadeca-9,12-dien-1-yloxy]ethoxy]oxolan-2-yl]-3H-pyrimidine-2,4-dione 45d and 1-[(2R,3R,4R,5R)-5-[[bis(4-methoxyphenyl)(phenyl)methoxy]methyl]-4-hydroxy-3-[2-[(9Z,12Z)-octadeca-9,12-dien-1-yloxy]ethoxy]oxolan-2-yl]-3H-pyrimidine-2,4-dione 46d: To a mixture of 2 (30.00 g, 63.141 mmol) in NMP (300.00 mL) was added (6Z,9Z)-18-(2-bromoethoxy)octadeca-6,9-diene d (47.16 g, 126.28 mmol) and NaI (18.93 g, 126.29 mmol). The resulting solution was stirred for 16 h at 90° C. The reaction mixture was cooled to rt and the resulting solution was diluted with 500 mL of water and extracted with pet ether (3×500 mL). The organic phase was dried over anhydrous Na₂SO₄, filtered and the filtrate was concentrated under vacuum. The crude residue was purified by silica gel column with EA/PE (2:1) which resulted in a mixture of 20 g (47.2%) 43d and 44d as a brown solid. This mixture was dissolved in pyridine (200.00 mL) and to this solution was added DMTrCl (25.60 g, 75.5 mmol, 4.0 eqiv). The resulting solution was stirred for 16 h at 25° C. The resulting solution was diluted with water (200 mL) and extracted with of DCM (3×200 mL). The combined organic layer was washed with brine (3×300 mL), separated, dried over anhydrous sodium sulfate and concentrated under vacuum. The crude residue was purified by a flash silica gel column chromatography with DCM/MeOH (20:1). The semi pure compound (20 g) was further was purified by Prep SFC which resulted in 5.2 (16%) g of 45d and 5.2 g (16%) of 46d as brown oil.

Compound 45d: ¹H NMR, (300 MHz, DMSO-d₆) δ 11.35 (d, J=2.3 Hz, 1H), 7.73 (d, J=8.1 Hz, 1H), 7.39-7.26 (m, 4H), 7.26-7.18 (m, 5H), 6.92-6.83 (m, 4H), 5.76-5.67 (m, 1H), 5.37-5.22 (m, 5H), 4.23 (t, J=4.1 Hz, 1H), 4.01 (qd, J=6.2, 4.0 Hz, 2H), 3.72 (s, 6H), 3.73-3.64 (m, 1H), 3.57 (dt, J=10.9, 4.9 Hz, 1H), 3.45 (t, J=4.9 Hz, 2H), 3.30 (t, J=6.6 Hz, 2H), 3.25 (d, J=3.2 Hz, 2H), 2.71 (t, J=6.0 Hz, 2H), 1.98 (qd, J=6.6, 2.4 Hz, 4H), 1.40 (t, J=6.7 Hz, 2H), 1.34-1.17 (m, 17H), 0.86-0.78 (m, 3H) ppm. ¹³C NMR (101 MHz, DMSO-d₆) δ 162.95, 158.12, 150.41, 144.59, 140.33, 135.30, 135.11, 129.71, 129.68, 129.66, 127.83, 127.70, 127.68, 127.66, 126.73, 113.20, 101.35, 89.11, 85.94, 80.56, 77.21, 72.23, 70.34, 69.57, 69.12, 62.43, 55.00, 30.87, 29.15, 29.00, 28.83, 28.81, 28.69, 28.59, 26.61, 26.58, 25.56, 25.18, 21.93, 13.85 ppm. HRMS calc. for C₅₀H₆₆N₂O₉Na [M+Na]861.4666, found 861.4658.

Compound 46d: ¹H NMR, (300 MHz, DMSO-d₆) δ 11.37 (d, J=2.2 Hz, 1H), 7.70 (d, J=8.1 Hz, 1H), 7.40-7.33 (m, 2H), 7.30 (dd, J=8.6, 6.7 Hz, 2H), 7.27-7.15 (m, 5H), 6.92-6.82 (m, 4H), 5.79 (d, J=3.7 Hz, 1H), 5.37-5.22 (m, 5H), 5.07 (d, J=6.6 Hz, 1H), 4.17 (q, J=6.0 Hz, 1H), 3.96 (ddd, J=15.8, 5.4, 3.3 Hz, 2H), 3.72 (s, 8H), 3.54-3.43 (m, 2H), 3.38-3.27 (m, 2H), 3.27 (dd, J=9.9, 3.6 Hz, 1H), 3.21 (dd, J=10.7, 2.9 Hz, 1H), 2.70 (t, J=6.0 Hz, 2H), 1.98 (qd, J=6.6, 3.2 Hz, 4H), 1.41 (q, J=5.5, 4.5 Hz, 2H), 1.35-1.24 (m, 3H), 1.23 (ddd, J=13.1, 6.2, 2.8 Hz, 13H), 0.86-0.77 (m, 3H) ppm. ¹³C NMR (101 MHz, DMSO-d₆) δ 162.93, 158.12, 150.28, 144.61, 140.26, 135.34, 135.08, 129.75, 129.70, 129.68, 127.86, 127.72, 127.69, 126.76, 113.22, 101.49, 87.18, 85.88, 82.57, 81.17, 70.35, 69.37, 69.26, 68.55, 62.68, 55.02, 30.88, 29.17, 29.01, 28.84, 28.70, 28.61, 26.61, 26.58, 25.58, 25.19, 21.94, 13.88 ppm. HRMS calc. for C₅₀H₆₆N₂O₉Na [M+Na]⁺ 861.4666, found 861.4673.

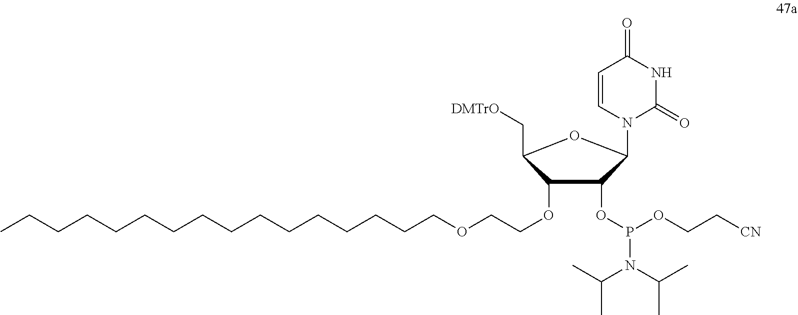

Synthesis of 3-[[(2R,5R)—S-[[bis(4-methoxyphenyl)-phenyl-methoxy]methyl]-2-(2,4-dioxopyrimidin-1-yl)-4-(2-hexadecoxyethoxy)tetrahydrofuran-3-yl]oxy-(diisopropylamino) phosphanyl]oxypropanenitrile 47a: Compound 45a (5.8 g, 7.12 mmol) was dissolved in DCM (100 mL) at 22° C. and stirred for 5 minutes. To this reaction mixture, DIPEA (4.64 g, 35.58 mmol, 6.26 mL) and N-methylimidazole (NMI) (2.07 g, 24.91 mmol, 2.01 mL) were added slowly. The resulting solution was stirred for 5 minutes after which 2-cyanoethyl-N,N-diisopropylchlorophosphoramidite (3.55 g, 14.23 mmol, 3.35 mL) was added in single portion. Reaction mixture was kept for 1 h stirring at 22° C. and TLC was checked. Reaction mixture was diluted with DCM (50 mL) and washed with 10% NaHCO₃ solution (2×50 mL). Organic layer separated, dried over anhydrous Na₂SO₄, filtered and the filtrate was evaporated to dryness. The crude mass obtained was purified by combiflash chromatography (Gradient: 20-50% EA in hexane) to afford 47a (5.8 g, 5.71 mmol, 80%) as a hygroscopic solid. ¹H NMR (400 MHz, CDCl₃, TMS, 25° C.) δ 8.35 (s, 1H), 8.03 (dd, J=11.6, 8.2 Hz, 1H), 7.42-7.05 (m, 9H), 6.93-6.67 (m, 4H), 6.10-5.82 (m, 1H), 5.29 (dd, J=13.6, 8.2 Hz, 1H), 4.47 (dtd, J=11.2, 4.0, 2.7 Hz, 1H), 4.23-4.14 (m, 2H), 3.96-3.76 (m, 10H), 3.65-3.27 (m, 9H), 2.74-2.53 (m, 2H), 1.51 (q, J=2.7 Hz, 2H), 1.34-1.21 (m, 28H), 1.20-1.14 (m, 12H), 0.92-0.83 (m, 3H) ppm. ¹³C NMR (101 MHz, CDCl₃, 25° C.) δ 163.02, 162.97, 158.86, 158.83, 158.81, 150.18, 149.99, 144.50, 144.48, 140.52, 140.41, 135.48, 135.43, 135.26, 135.20, 130.28, 130.24, 128.26, 128.25, 128.17, 128.14, 127.29, 127.25, 118.11, 117.93, 113.45, 113.42, 102.11, 102.06, 88.92, 87.23, 87.15, 81.71, 81.69, 76.06, 75.90, 75.14, 74.99, 71.80, 71.75, 70.42, 70.32, 61.79, 58.83, 58.66, 58.49, 58.30, 55.38, 43.65, 43.55, 43.52, 43.42, 32.07, 29.85, 29.80, 29.66, 29.64, 29.51, 26.24, 26.22, 24.88, 24.81, 24.79, 24.73, 24.67, 24.61, 24.53, 22.84, 20.53, 20.46, 20.44, 20.37, 14.27 ppm. ³¹P NMR (162 MHz, CDCl₃, 25° C.) δ 150.82, 150.07 ppm. HRMS calc. for C57H₈₄N₄O₁₀P [M+H]⁺ 1015.5925, found 1015.5919.

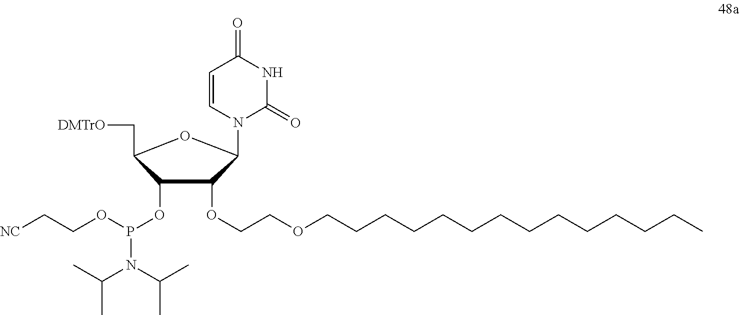

Synthesis of 3-[[(2R,5R)-2-[[bis(4-methoxyphenyl)-phenyl-methoxy]methyl]-5-(2,4-dioxopyrimidin-1-yl)-4-(2-hexadecoxyethoxy)tetrahydrofuran-3-yl]oxy-(diisopropylamino) phosphanyl]oxypropanenitrile 48a: Compound 46a (5.20 g, 6.38 mmol) was dissolved in DCM (100 mL) at 22° C. and stirred for 5 minutes. To this reaction mixture, DIPEA (4.16 g, 31.90 mmol, 5.61 mL) and NMI (1.85 g, 22.33 mmol, 1.80 mL) were added slowly. The resulting solution was stirred for 5 minutes after which 2-cyanoethyl-N,N-diisopropylchlorophosphoramidite (3.18 g, 12.76 mmol, 3.00 mL) was added in single portion. Reaction mixture was kept stirring for 1 h at 22° C. and TLC was checked. Reaction mixture was diluted with DCM (50 mL) and washed with 10% NaHCO₃ solution (2×50 mL). Organic layer separated, dried over anhydrous Na₂SO₄, filtered and the filtrate was evaporated to dryness. The crude mass obtained was purified by combiflash chromatography (Gradient: 20-50% EA in hexane) to afford 48a (5.2 g, 5.12 mmol, 80% yield) as a hygroscopic solid. ¹H NMR (400 MHz, CDCl₃, TMS, 25° C.) δ 8.38 (s, 1H), 8.01 (dd, J=34.2, 8.2 Hz, 1H), 7.49-7.17 (m, 9H), 6.84 (ddd, J=9.0, 6.7, 2.3 Hz, 4H), 5.98 (dd, J=16.6, 2.6 Hz, 1H), 5.22 (dd, J=14.2, 8.1 Hz, 1H), 4.52 (dddd, J=49.1, 9.8, 7.0, 4.8 Hz, 1H), 4.32-4.18 (m, 1H), 4.10 (ddd, J=9.7, 4.8, 2.7 Hz, 1H), 3.98-3.88 (m, 2H), 3.87-3.76 (m, 8H), 3.59 (tdd, J=9.6, 4.5, 2.9 Hz, 6H), 3.43 (td, J=6.8, 3.0 Hz, 3H), 2.64 (td, J=6.2, 4.2 Hz, 1H), 2.42 (t, J=6.3 Hz, 1H), 1.62-1.42 (m, 2H), 1.25 (d, J=4.4 Hz, 28H), 1.20-1.13 (m, 9H), 1.03 (d, J=6.8 Hz, 3H), 0.93-0.80 (m, 3H) ppm. ¹³C NMR (101 MHz, CDCl₃, 25° C.) δ 163.27, 163.19, 158.87, 158.84, 150.21, 150.14, 144.47, 144.31, 140.37, 140.32, 135.38, 135.29, 135.21, 135.09, 130.43, 130.41, 128.44, 128.09, 127.33, 117.92, 117.57, 113.36, 113.34, 102.17, 102.04, 88.32, 88.13, 87.26, 87.06, 82.54, 82.51, 82.31, 82.28, 81.86, 81.83, 71.70, 71.67, 70.50, 70.24, 70.10, 70.03, 69.99, 69.88, 61.61, 60.98, 58.88, 58.71, 58.17, 57.97, 55.40, 55.37, 53.57, 43.48, 43.40, 43.35, 43.27, 32.06, 29.84, 29.79, 29.68, 29.50, 26.24, 24.83, 24.78, 24.75, 24.71, 24.64, 22.83, 20.62, 20.56, 20.45, 20.38, 14.27 ppm. ³¹P NMR (162 MHz, CDCl₃, 25° C.) δ 151.18, 151.17 ppm. HRMS calc. for C57H₈₄N₄O₁₀P [M+H]⁺ 1015.5925, found 1015.5933.

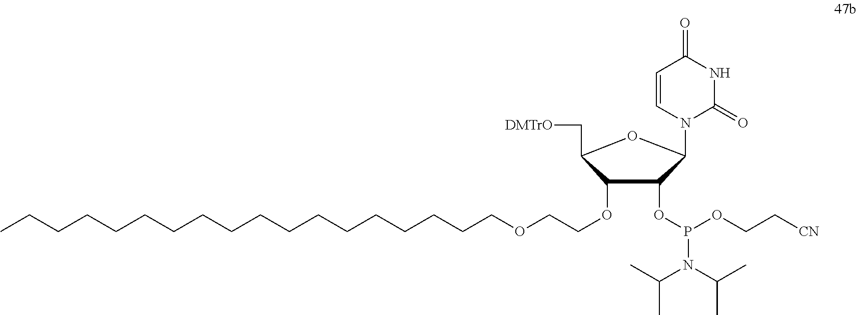

Synthesis of 3-[[(2R,5R)-5-[[bis(4-methoxyphenyl)-phenyl-methoxy]methyl]-2-(2,4-dioxopyrimidin-1-yl)-4-(2-octadecoxyethoxy)tetrahydrofuran-3-yl]oxy-(diisopropylamino) phosphanyl]oxypropanenitrile 47b: To a solution of 45b (3.1 g, 3.68 mmol) was dissolved in DCM (30 mL) at 22° C. and stirred for 5 minutes. To this reaction mixture, DIPEA (2.40 g, 18.38 mmol, 3.23 mL) and NMI (1.07 g, 12.87 mmol, 1.04 mL) were added slowly. The resulting solution was stirred for 5 minutes after which 2-cyanoethyl-N,N-diisopropylchlorophosphoramidite (1.83 g, 7.35 mmol) was added in single portion. Reaction mixture was kept stirring for 1 h at 22° C. and TLC was checked. Reaction mixture was diluted with DCM (30 mL) and washed with 10% NaHCO₃ solution (2×50 mL). Organic layer separated, dried over anhydrous Na₂SO₄, filtered and the filtrate was evaporated to dryness. The crude mass obtained was purified by combiflash chromatography (Gradient: 20-50% EA in hexane) to afford 47b (3.1 g, 2.97 mmol, 81%) as a hygroscopic solid. ¹H NMR (500 MHz, CDCl₃, TMS, 25° C.) δ 8.53 (s, 1H), 8.02 (dd, J=12.9, 8.2 Hz, 1H), 7.47-7.12 (m, 9H), 6.84 (dd, J=8.9, 3.3 Hz, 4H), 6.03 (dd, J=34.0, 2.8 Hz, 1H), 5.30 (dd, J=16.8, 8.1 Hz, 1H), 4.47 (ddt, J=8.8, 5.0, 3.0 Hz, 1H), 4.25-4.16 (m, 2H), 3.98-3.74 (m, 10H), 3.70-3.52 (m, 6H), 3.48-3.36 (m, 3H), 2.73-2.56 (m, 2H), 1.51 (q, J=6.6 Hz, 2H), 1.25 (d, J=5.3 Hz, 33H), 1.22-1.15 (m, 12H), 0.88 (t, J=6.9 Hz, 3H) ppm. ¹³C NMR (101 MHz, CDCl₃, 25° C.) δ 163.06, 163.00, 158.86, 158.83, 158.81, 150.21, 150.00, 144.50, 144.48, 140.52, 140.41, 135.49, 135.43, 135.27, 135.20, 130.28, 130.24, 128.27, 128.25, 128.17, 128.14, 127.29, 127.25, 118.12, 117.93, 113.46, 113.42, 102.12, 102.06, 88.92, 87.23, 87.16, 81.71, 81.69, 76.06, 75.90, 75.14, 74.99, 71.80, 71.75, 70.40, 70.32, 61.80, 58.83, 58.66, 58.50, 58.31, 55.38, 43.65, 43.55, 43.53, 43.42, 29.85, 29.80, 29.66, 29.64, 29.50, 26.24, 26.22, 24.88, 24.81, 24.79, 24.73, 24.67, 24.61, 24.54, 22.83, 20.52, 20.46, 20.37, 14.26 ppm. ³¹P NMR (202 MHz, CDCl₃, 25° C.) 6 151.94, 151.15 ppm. HRMS calc. for C₅₉H₈₈N₄O₁₀P [M+H]⁺ 1043.6238, found 1043.6239.

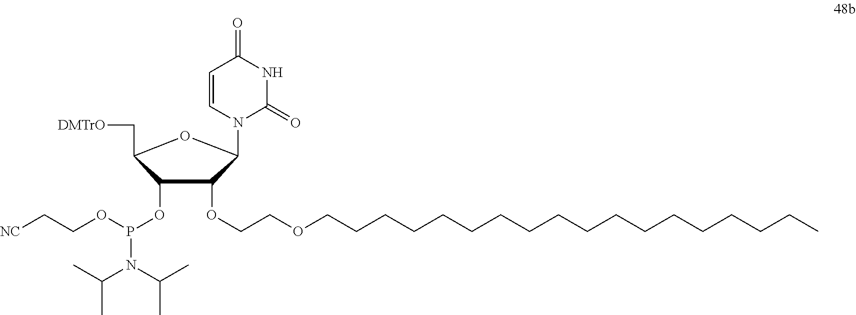

Synthesis of 3-[[(2R,5R)-2-[[bis(4-methoxyphenyl)-phenyl-methoxy]methyl]-5-(2,4-dioxopyrimidin-1-yl)-4-(2-octadecoxyethoxy)tetrahydrofuran-3-yl]oxy-(diisopropylamino) phosphanyl]oxypropanenitrile 48b: To a clear solution of 46b (2.96 g, 3.51 mmol) in DCM (30 mL) at 22° C. were added DIPEA (2.29 g, 17.55 mmol, 3.09 mL, 99% purity) and NMI (1.02 g, 12.29 mmol, 989.36 μL, 99% purity) slowly. The resulting solution was stirred for 5 minutes and 2-cyanoethyl-N,N-diisopropylchlorophosphoramidite (1.75 g, 7.02 mmol, 95% purity) was added in single portion. Reaction mixture was kept stirring for 1 h at 22° C. and TLC was checked. Reaction mixture was diluted with DCM (30 mL) and washed with 10% NaHCO₃ solution (2×50 mL). Organic layer separated, dried over anhydrous Na₂SO₄, filtered and the filtrate was evaporated to dryness. The crude mass obtained was purified by combiflash chromatography (Gradient: 20-50% EA in hexane) to afford 48b (3.1 g, 2.97 mmol, 85%) as a hygroscopic solid. ¹H NMR (500 MHz, CDCl₃, TMS, 25° C.) δ 8.53 (s, 1H), 8.00 (dd, J=43.1, 8.2 Hz, 1H), 7.48-7.10 (m, 9H), 6.84 (td, J=8.7, 2.8 Hz, 4H), 5.98 (dd, J=20.6, 2.6 Hz, 1H), 5.22 (dd, J=16.9, 8.1 Hz, 1H), 4.52 (dddd, J=60.9, 9.9, 7.0, 4.8 Hz, 1H), 4.25 (ddd, J=26.3, 5.7, 2.5 Hz, 1H), 4.10 (ddd, J=12.6, 4.8, 2.7 Hz, 1H), 3.99-3.87 (m, 1H), 3.86-3.77 (m, 8H), 3.74-3.51 (m, 6H), 3.51-3.34 (m, 3H), 2.63 (q, J=6.0 Hz, 1H), 2.42 (t, J=6.3 Hz, 1H), 1.63-1.44 (m, 2H), 1.25 (d, J=5.8 Hz, 32H), 1.21-1.12 (m, 9H), 1.03 (d, J=6.8 Hz, 3H), 0.88 (t, J=6.9 Hz, 3H) ppm. ¹³C NMR (101 MHz, CDCl₃, 25° C.) δ 163.26, 163.18, 158.89, 158.87, 158.85, 150.22, 150.15, 144.48, 144.32, 140.36, 140.32, 135.40, 135.32, 135.23, 135.12, 130.43, 130.41, 128.45, 128.09, 127.33, 117.90, 117.56, 113.38, 113.36, 102.17, 102.04, 88.33, 88.15, 87.27, 87.08, 82.54, 82.52, 82.34, 82.30, 81.87, 81.84, 71.70, 71.67, 70.51, 70.25, 70.11, 70.07, 70.00, 61.65, 61.03, 58.89, 58.72, 58.19, 57.99, 55.40, 55.37, 43.50, 43.42, 43.37, 43.30, 32.06, 29.84, 29.80, 29.68, 29.50, 26.25, 24.83, 24.76, 24.72, 24.64, 22.83, 20.62, 20.56, 20.45, 20.38, 14.26 ppm. ³¹P NMR (202 MHz, CDCl₃, 25° C.) δ 150.73, 150.73 ppm. HRMS calc. for C₅₉H₈₈N₄O₁₀P [M+H]⁺ 1043.6238, found 1043.6229.

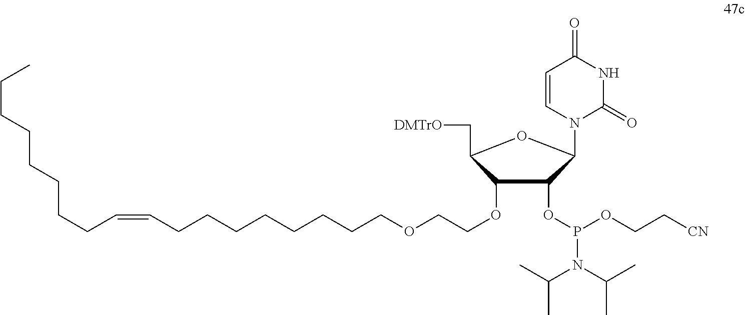

Synthesis of 3-[[(2R,5R)-5-[[bis(4-methoxyphenyl)-phenyl-methoxy]methyl]-2-(2,4-dioxopyrimidin-1-yl)-4-[2-[(Z)-octadec-9-enoxy]ethoxy]tetrahydrofuran-3-yl]oxy-(diisopropyl amino)phosphanyl]oxypropanenitrile 47c: To a solution of compound 45c (3 g, 3.57 mmol) in DCM (30 mL) at 22° C. was added DIPEA (2.33 g, 17.83 mmol, 3.14 mL, 99% purity) and NMI (1.04 g, 12.48 mmol, 1.01 mL, 99% purity) slowly. The resulting solution was stirred for 5 minutes and 2-cyanoethyl-N,N-diisopropylchlorophosphoramidite (1.78 g, 7.13 mmol, 95% purity) was added in single portion. Reaction mixture was kept for 1.5 h stirring at 22° C. and TLC was checked. Reaction mixture was diluted with DCM (50 mL) and washed with 10% NaHCO₃ solution (2×50 mL). Organic layer separated, dried over anhydrous Na₂SO₄, filtered and the filtrate was evaporated to dryness. The crude mass obtained was purified by combiflash chromatography (Gradient: 20-50% EA in hexane) to afford 47c (3.0 g, 2.88 mmol, 81%) as a hygroscopic solid. ¹H NMR (400 MHz, CDCl₃, TMS, 25° C.) δ 8.36 (s, 0.5H), 8.22 (s, 0.5H), 8.03 (dd, J=11.6, 8.1 Hz, 1H), 7.49-7.13 (m, 9H), 6.96-6.69 (m, 4H), 6.30-5.80 (m, 1H), 5.58-5.05 (m, 3H), 4.47 (ddt, J=8.7, 4.9, 2.8 Hz, 1H), 4.27-4.15 (m, 2H), 3.97-3.73 (m, 9H), 3.72-3.50 (m, 6H), 3.49-3.34 (m, 3H), 2.79-2.47 (m, 2H), 2.15-1.88 (m, 4H), 1.51 (tt, J=7.0, 3.6 Hz, 2H), 1.37-1.23 (m, 24H), 1.22-1.12 (m, 12H), 0.96-0.80 (m, 3H). ppm. ¹³C NMR (126 MHz, CDCl₃, 25° C.) δ 162.89, 162.84, 158.88, 158.85, 158.83, 150.09, 149.94, 144.50, 144.49, 140.53, 140.41, 135.50, 135.44, 135.28, 135.22, 130.29, 130.25, 130.10, 129.96, 128.28, 128.26, 128.18, 128.15, 127.30, 127.27, 118.08, 117.92, 113.47, 113.44, 102.09, 102.06, 88.97, 88.93, 87.25, 87.17, 81.74, 81.72, 76.04, 75.92, 75.16, 75.03, 71.80, 71.75, 70.43, 70.34, 61.82, 58.82, 58.68, 58.46, 58.31, 55.39, 43.65, 43.55, 43.45, 32.05, 29.93, 29.85, 29.82, 29.70, 29.68, 29.64, 29.62, 29.48, 29.46, 29.45, 27.38, 26.25, 26.23, 24.88, 24.83, 24.81, 24.74, 24.69, 24.61, 24.55, 22.83, 20.52, 20.48, 20.43, 20.37, 14.26 ppm. ³¹P NMR (162 MHz, CDCl₃, 25° C.) δ 151.28, 150.55 ppm. HRMS calc. for C₅₉H₈₆N₄O₁₀P [M+H]⁺ 1041.6082, found 1041.6082.

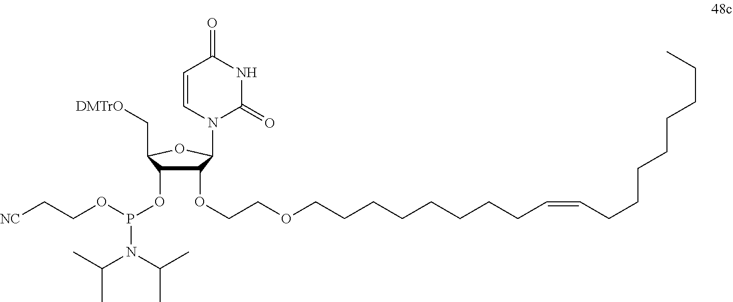

Synthesis of 3-[[(2R,5R)-2-[[bis(4-methoxyphenyl)-phenyl-methoxy]methyl]-5-(2,4-dioxopyrimidin-1-yl)-4-[2-[(Z)-octadec-9-enoxy]ethoxy]tetrahydrofuran-3-yl]oxy-(diisopropyl amino)phosphanyl]oxypropanenitrile 48c: To a clear solution of 46c (3.0 g, 3.57 mmol) in DCM (30 mL) at 22° C. was added DIPEA (2.33 g, 17.83 mmol, 3.14 mL) and NMI (1.04 g, 12.48 mmol, 1.01 mL) slowly. The resulting solution was stirred for 5 minutes after which 2-cyanoethyl-N,N-diisopropylchlorophosphoramidite (1.78 g, 7.13 mmol, 1.68 mL, 95% purity) was added in single portion. Reaction mixture was kept for 1.5 h stirring at 22° C. and TLC was checked. Reaction mixture was diluted with DCM (50 mL) and washed with 10% NaHCO₃ solution (2×50 mL). Organic layer separated, dried over anhydrous Na₂SO₄, filtered and the filtrate was evaporated to dryness. The crude mass obtained was purified by combiflash chromatography (Gradient: 20-50% EA in hexane) to afford 48c (3.1 g, 2.98 mmol, 83% yield) as a hygroscopic solid. ¹H NMR (400 MHz, CDCl₃, TMS, 25° C.) δ 8.55 (s, 1H), 8.01 (dd, J=34.4, 8.1 Hz, 1H), 7.62-7.11 (m, 9H), 6.84 (ddd, J=9.0, 6.7, 2.3 Hz, 4H), 5.98 (dd, J=16.8, 2.6 Hz, 1H), 5.43-5.28 (m, 2H), 5.22 (dd, J=14.1, 8.1 Hz, 1H), 4.52 (dddd, J=49.7, 9.8, 7.1, 4.8 Hz, 1H), 4.25 (ddt, J=21.1, 6.7, 2.4 Hz, 1H), 4.10 (ddd, J=9.5, 4.9, 2.6 Hz, 1H), 3.99-3.87 (m, 2H), 3.87-3.75 (m, 8H), 3.73-3.51 (m, 7H), 3.44 (ddt, J=10.1, 6.8, 2.7 Hz, 3H), 2.64 (td, J=6.3, 4.2 Hz, 1H), 2.42 (t, J=6.3 Hz, 1H), 2.01 (h, J=5.5, 3.8 Hz, 4H), 1.52 (q, J=6.4, 6.0 Hz, 2H), 1.27 (d, J=6.2 Hz, 24H), 1.21-1.09 (m, 10H), 1.03 (d, J=6.7 Hz, 3H), 0.94-0.78 (m, 3H) ppm. ¹³C NMR (126 MHz, CDCl₃, 25° C.) 6 163.45, 163.36, 158.87, 158.85, 158.83, 150.29, 150.21, 144.47, 144.31, 140.34, 140.29, 135.38, 135.30, 135.21, 135.11, 130.42, 130.40, 130.03, 129.96, 128.43, 128.07, 127.30, 117.89, 117.54, 113.36, 113.33, 102.17, 102.03, 88.33, 88.14, 87.25, 87.06, 82.52, 82.47, 82.28, 82.25, 81.86, 81.84, 71.66, 71.63, 70.49, 70.24, 70.10, 70.01, 69.99, 69.90, 69.88, 61.62, 60.98, 58.86, 58.72, 58.17, 58.00, 55.38, 55.35, 43.46, 43.39, 43.37, 43.29, 32.02, 29.89, 29.82, 29.76, 29.73, 29.66, 29.64, 29.63, 29.56, 29.44, 29.42, 27.34, 26.22, 24.81, 24.77, 24.75, 24.69, 24.63, 22.80, 20.60, 20.55, 20.43, 20.37, 14.24 ppm. ³¹P NMR (162 MHz, CDCl₃, 25° C.) δ 150.75, 150.74 ppm. HRMS calc. for C₅₉H₈₆N₄O₁₀P [M+H]⁺ 1041.6082, found 1041.6082.

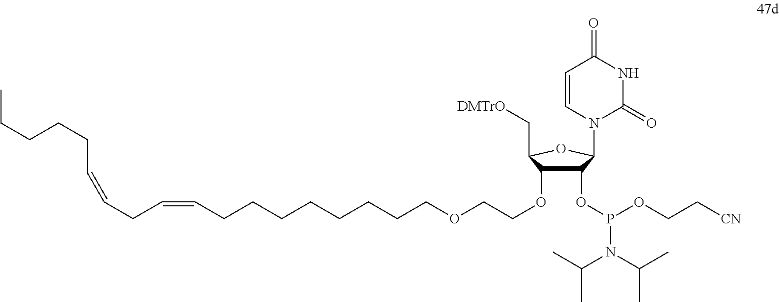

Synthesis of 3-[[(2R,5R)-5-[[bis(4-methoxyphenyl)-phenyl-methoxy]methyl]-2-(2,4-dioxopyrimidin-1-yl)-4-[2-[(9Z,12Z)-octadeca-9,12-dienoxy]ethoxy]tetrahydrofuran-3-yl]oxy-(diisopropylamino)phosphanyl]oxypropanenitrile 47d: To a clear solution of 45d (2.9 g, 3.46 mmol) was dissolved in DCM (20 mL) at 22° C. and stirred for 5 minutes. To this reaction mixture, DIPEA (2.26 g, 17.28 mmol, 3.04 mL) and N-methyl imidazoleI (1.00 g, 12.10 mmol, 973.96 μL) were added slowly. The resulting solution was stirred for 5 minutes after which 2-cyanoethyl-N,N-diisopropylchlorophosphoramidite (1.72 g, 6.91 mmol, 1.62 mL) was added in single portion. Reaction mixture was kept for 1 h stirring at 22° C. and TLC was checked. Reaction mixture was diluted with DCM (50 mL) and washed with 10% NaHCO₃ solution (2×50 mL). Organic layer separated, dried over anhydrous Na₂SO₄, filtered and the filtrate was evaporated to dryness. The crude mass obtained was purified by combiflash chromatography (Gradient: 20-50% EA in hexane) to afford 47d (2.9 g, 2.79 mmol, 81% yield) as transparent gum. ¹H NMR (400 MHz, CDCl₃, TMS, 25° C.) δ 8.54 (s, 1H), 8.03 (dd, J=10.3, 8.2 Hz, 1H), 7.52-7.15 (m, 9H), 6.92-6.68 (m, 4H), 6.19-5.87 (m, 1H), 5.51-5.12 (m, 5H), 4.47 (ddd, J=10.7, 5.9, 2.6 Hz, 1H), 4.28-4.13 (m, 2H), 3.98-3.73 (m, 10H), 3.68-3.51 (m, 6H), 3.42 (dqd, J=19.7, 6.4, 1.7 Hz, 3H), 2.83-2.72 (m, 2H), 2.71-2.53 (m, 2H), 2.15-1.97 (m, 4H), 1.59-1.45 (m, 2H), 1.40-1.14 (m, 33H), 0.97-0.78 (m, 3H) ppm. ¹³C NMR (126 MHz, CDCl₃, 25° C.) δ 163.12, 163.06, 158.86, 158.83, 158.81, 150.26, 150.04, 144.49, 144.48, 140.50, 140.40, 135.49, 135.43, 135.27, 135.20, 130.34, 130.27, 130.24, 130.15, 128.26, 128.25, 128.19, 128.16, 128.13, 128.06, 127.28, 127.25, 118.12, 117.92, 113.45, 113.42, 113.42, 102.14, 102.07, 88.94, 88.90, 87.23, 87.15, 81.70, 76.03, 75.91, 75.12, 75.00, 71.78, 71.73, 70.40, 70.38, 70.31, 70.29, 61.82, 61.80, 58.81, 58.67, 58.49, 58.34, 55.37, 43.64, 43.54, 43.44, 31.66, 29.83, 29.82, 29.80, 29.68, 29.67, 29.61, 29.59, 29.48, 29.43, 27.38, 27.34, 26.23, 26.21, 25.77, 24.86, 24.81, 24.79, 24.72, 24.67, 24.60, 24.54, 22.71, 20.51, 20.46, 20.43, 20.37, 14.21 ppm. ³¹P NMR (162 MHz, CDCl₃, 25° C.) δ 152.38, 151.60 ppm. HRMS calc. for C₅₉H₈₄N₄O₁₀P [M+H]⁺ 1039.5925, found 1039.5941.

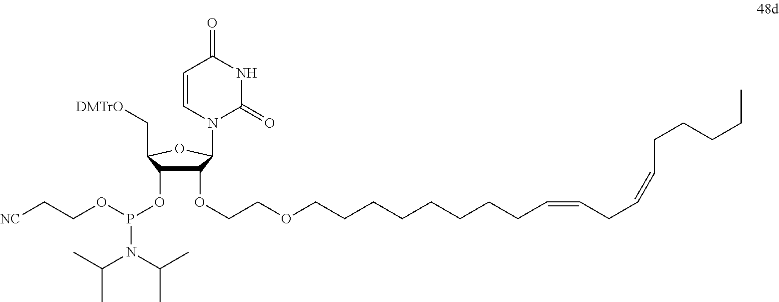

Synthesis of 3-[[(2R,5R)-2-[[bis(4-methoxyphenyl)-phenyl-methoxy]methyl]-5-(2,4-dioxopyrimidin-1-yl)-4-[2-[(9Z,12Z)-octadeca-9,12-dienoxy]ethoxy]tetrahydrofuran-3-yl]oxy-(diisopropylamino)phosphanyl]oxypropanenitrile 48d: To a clear solution of 46d (2.75 g, 3.28 mmol) in DCM (20 mL) at 22° C. was added DIPEA (2.14 g, 16.39 mmol, 2.88 mL) and NMI (951.29 mg, 11.47 mmol, 923.58 μL) slowly. The resulting solution was stirred for 5 minutes after which 2-cyanoethyl-N,N-diisopropylchlorophosphoramidite (1.63 g, 6.55 mmol, 1.54 mL) was added in single portion. Reaction mixture was kept for 1 h stirring at 22° C. and TLC was checked. Reaction mixture was diluted with DCM (50 mL) and washed with 10% NaHCO₃ solution (2×50 mL). Organic layer separated, dried over anhydrous Na₂SO₄, filtered and the filtrate was evaporated to dryness. The crude mass obtained was purified by combiflash chromatography (Gradient: 20-50% EA in hexane) to afford 48d (2.81 g, 2.70 mmol, 83%) as transparent gum. ¹H NMR (400 MHz, CDCl₃, TMS, 25° C.) δ 8.62 (s, 1H), 8.01 (dd, J=34.8, 8.2 Hz, 1H), 7.67-7.08 (m, 9H), 6.84 (ddd, J=9.0, 6.7, 2.3 Hz, 4H), 5.98 (dd, J=16.9, 2.6 Hz, 1H), 5.43-5.27 (m, 4H), 5.22 (dd, J=13.8, 8.1 Hz, 1H), 4.52 (dddd, J=49.9, 9.8, 7.1, 4.8 Hz, 1H), 4.25 (ddt, J=21.2, 6.8, 2.4 Hz, 1H), 4.09 (ddd, J=9.6, 4.9, 2.6 Hz, 1H), 3.94 (dddd, J=10.9, 9.6, 5.7, 2.7 Hz, 2H), 3.87-3.78 (m, 8H), 3.71-3.52 (m, 6H), 3.48-3.38 (m, 3H), 2.77 (t, J=6.5 Hz, 2H), 2.64 (td, J=6.4, 4.0 Hz, 1H), 2.42 (t, J=6.3 Hz, 1H), 2.16-1.95 (m, 4H), 1.66-1.45 (m, 2H), 1.47-1.23 (m, 18H), 1.22-1.10 (m, 9H), 1.03 (d, J=6.8 Hz, 3H), 0.95-0.80 (m, 3H) ppm. ¹³C NMR (126 MHz, CDCl₃, 25° C.) δ 163.24, 163.16, 158.89, 158.87, 158.85, 150.20, 150.13, 144.48, 144.32, 140.36, 140.32, 135.40, 135.31, 135.23, 135.12, 130.43, 130.41, 130.33, 130.27, 128.45, 128.10, 128.09, 128.07, 127.32, 117.89, 117.56, 113.38, 113.35, 113.25, 102.16, 102.04, 88.33, 88.16, 87.27, 87.08, 82.55, 82.53, 82.50, 82.33, 82.30, 81.87, 81.85, 71.68, 71.66, 70.51, 70.25, 70.11, 70.04, 70.00, 69.94, 69.91, 61.63, 61.01, 58.86, 58.72, 58.16, 58.00, 55.40, 43.48, 43.41, 43.38, 43.31, 31.66, 29.84, 29.82, 29.67, 29.64, 29.48, 29.44, 27.38, 27.34, 26.24, 25.77, 24.82, 24.78, 24.76, 24.71, 24.65, 22.71, 20.62, 20.57, 20.45, 20.39, 14.21 ppm. 31P NMR (162 MHz, CDCl₃, 25° C.) 6 151.18, 151.17 ppm. HRMS calc. for C₅₉H₈₄N₄O₁₀P [M+H]⁺ 1039.5925, found 1039.5923.

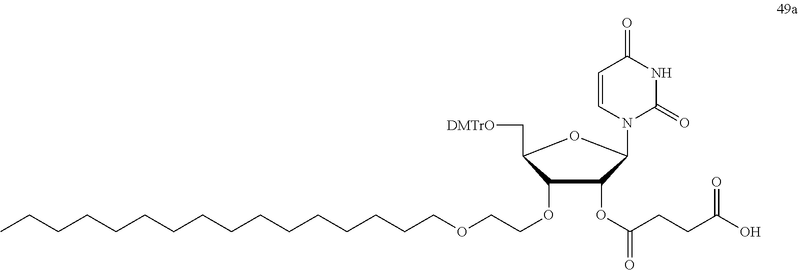

4-[(2R,5R)-5-[[bis(4-methoxyphenyl)-phenyl-methoxy]methyl]-2-(2,4-dioxopyrimidin-1-yl)-4-(2-hexadecoxyethoxy)tetrahydrofuran-3-yl]oxy-4-oxo-butanoic acid 49a: To a solution of 45a (0.5 g, 0.61 mmol) in DCM (15 mL), 4-(dimethylamino)pyridine (227 mg, 1.84 mmol) and succinic anhydride (124 mg, 1.23 mmol) were added. The resulting mixture was stirred for 3 hrs at 22° C. All the volatile matters were evaporated to dryness and crude mass which was dissolved in EA (30 mL). The organic layer was washed with 10% NH₄Cl solution (3×20 mL), and brine (20 mL). The organic layer was separated, dried over anhydrous Na₂SO₄, filtered and the filtrate was evaporated to dryness. The crude mass thus obtained was purified by combiflash chromatography (Gradient: 0-5% Methanol in DCM) for afford 49a (0.39 g, 69% yield) as white solid. ¹H NMR (400 MHz, Chloroform-d) δ 10.11 (s, 1H), 7.84 (d, J=8.2 Hz, 1H), 7.42-7.15 (m, 9H), 6.96-6.64 (m, 4H), 6.13 (d, J=4.0 Hz, 1H), 5.45 (dd, J=5.1, 4.1 Hz, 1H), 5.37 (d, J=8.2 Hz, 1H), 4.28 (t, J=5.5 Hz, 1H), 4.18 (dt, J=5.4, 2.4 Hz, 1H), 3.79 (s, 6H), 3.76-3.65 (m, 1H), 3.59-3.52 (m, 2H), 3.49 (t, J=4.6 Hz, 2H), 3.45-3.32 (m, 3H), 2.83-2.59 (m, 4H), 1.51 (q, J=6.9 Hz, 2H), 1.25 (d, J=4.1 Hz, 27H), 0.96-0.80 (m, 3H) ppm. ¹³C NMR (101 MHz, CDCl₃) δ 175.33, 171.08, 163.95, 158.86, 158.84, 150.80, 144.29, 140.13, 135.37, 135.17, 130.25, 130.23, 128.27, 128.18, 127.31, 113.46, 102.79, 87.25, 87.23, 82.24, 74.61, 71.75, 70.91, 70.20, 62.03, 55.38, 32.06, 29.84, 29.82, 29.79, 29.76, 29.70, 29.64, 29.50, 29.36, 29.17, 26.17, 22.83, 14.26 ppm. HRMS calc. for C₅₂H₇₀N₂O₁₂Na [M+Na]⁺ 937.4826, found 937.4813.

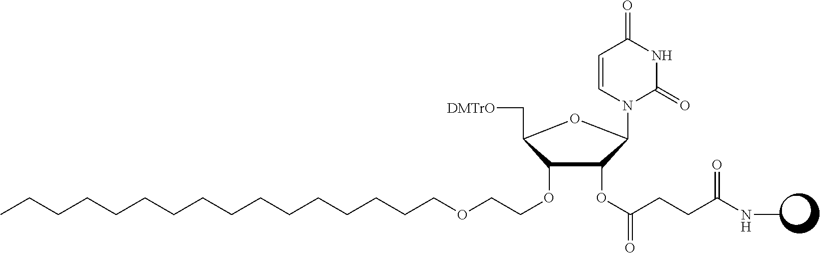

CPG from 49a: Added 4-[(2R,5R)-5-[[bis(4-methoxyphenyl)-phenyl-methoxy]methyl]-2-(2,4-dioxopyrimidin-1-yl)-4-(2-hexadecoxyethoxy)tetrahydrofuran-3-yl]oxy-4-oxo-butanoic acid (0.3 g, 327.83 mol) and N-ethyl-N-isopropyl-propan-2-amine (169.47 mg, 1.31 mmol, 228.40 μL) into rb flask. Then added dry acetonitrile (50 mL). Stirred well to dissolve and then HBTU (130.54 mg, 344.22 μmol) to preactivate acid. Let stir for ˜5 min, then added CPG. Capped and put on mechanical shaker overnight. Next day filtered CPG, washed with ACN, then MeOH, then ACN, then diethyl ether. Dried for ˜5 minutes, then transferred back to rb flask for capping. Added 30% acetic anhydride in pyridine (50 ml total) and 1% TEA. Capped and put back on mechanical shaker for 3 hours. After 3 hours took off and washed CPG as follows: 10% H₂O/THF, then MeOH, then 10% H₂O/THF, then MeOH, then ACN, the ether (˜250 mL each solvent for washing). Transferred to rb flask and dried CPG in high vacuum overnight.

Checking the Loading: Weighted out 40 mg and loaded into 250 ml volumetric flask. Then added 0.1M toluene-p-sulfonic acid in ACN up to measure line. Sonicated and settled for 1 hour. Checked loading by spectrophotometer and beers law. Measured solution into UV cuvette and measured UV absorbance at 411 nm. Check worksheet for raw data. Calculated loading using beers law=[250 (mL)×(absorbance A)×35.5 (extinction coefficient of DMTr)]/weight of CPG (mg). Yield: 1.82 g; Loading: 110 μmol/g.

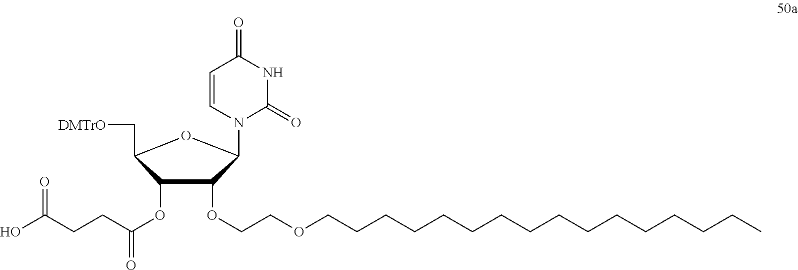

4-[(2R,5R)-2-[[bis(4-methoxyphenyl)-phenyl-methoxy]methyl]-5-(2,4-dioxopyrimidin-1-yl)-4-(2-hexadecoxyethoxy)tetrahydrofuran-3-yl]oxy-4-oxo-butanoic acid 50a: To a solution of 46a (0.6 g, 0.74 mmol) in DCM (15 mL), 4-(dimethylamino)pyridine (272 mg, 2.21 mmol) and succinic anhydride (148 mg, 1.47 mmol) were added. The resulting mixture was stirred for 3 hrs at 22° C. All the volatile matters were evaporated to dryness and crude mass which was dissolved in EA (30 mL). The organic layer was washed with 10% NH₄Cl solution (2×30 mL), and brine (20 mL). The organic layer was separated, dried over anhydrous Na₂SO₄, filtered and the filtrate was evaporated to dryness. The crude mass thus obtained was purified by combiflash chromatography (Gradient: 0-5% Methanol in DCM) for afford 50a (0.55 g, 82% yield) as white solid. ¹H NMR (400 MHz, Chloroform-d) δ 9.80 (s, 1H), 8.05 (d, J=8.1 Hz, 1H), 7.39-7.17 (m, 10H), 6.90-6.63 (m, 4H), 5.93 (d, J=2.1 Hz, 1H), 5.31 (dd, J=8.3, 1.3 Hz, 1H), 5.23 (dd, J=7.8, 5.0 Hz, 1H), 4.34 (dt, J=7.8, 2.3 Hz, 1H), 4.16 (dd, J=5.0, 2.2 Hz, 1H), 3.91 (ddd, J=11.1, 5.6, 2.8 Hz, 1H), 3.79 (d, J=0.8 Hz, 7H), 3.66-3.58 (m, 4H), 3.50-3.36 (m, 3H), 2.79-2.39 (m, 4H), 1.54 (q, J=7.0 Hz, 2H), 1.24 (d, J=6.6 Hz, 24H), 1.00-0.75 (in, 3H) ppm. ¹³C NMR (101 MHz, CDCl₃) δ 175.46, 171.55, 164.55, 158.87, 158.85, 149.94, 144.29, 140.51, 135.15, 135.09, 130.33, 130.27, 129.26, 128.23, 128.17, 127.32, 113.45, 113.30, 101.87, 88.37, 87.44, 81.18, 80.56, 71.64, 70.82, 70.27, 69.68, 60.91, 55.39, 32.06, 29.85, 29.83, 29.80, 29.78, 29.65, 29.58, 29.50, 29.18, 29.03, 26.16, 22.83, 14.26 ppm. HRMS calc. for C₅₂H₇₀N₂O₁₂Na [M+Na]⁺ 937.4826, found 937.4815.

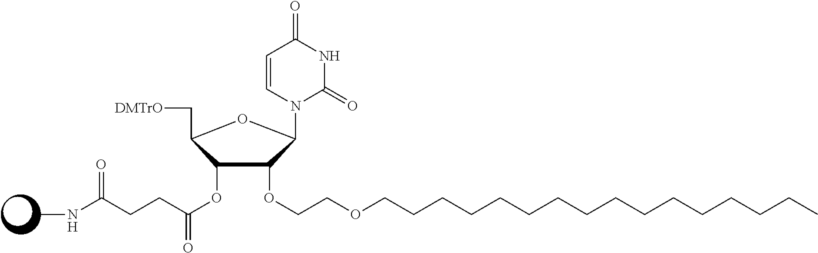

CPG from 50a: Added 4-[(2R,5R)-2-[[bis(4-methoxyphenyl)-phenyl-methoxy]methyl]-5-(2,4-dioxopyrimidin-1-yl)-4-(2-hexadecoxyethoxy)tetrahydrofuran-3-yl]oxy-4-oxo-butanoic acid (0.5 g, 546.38 mol) and N-ethyl-N-isopropyl-propan-2-amine (282.46 mg, 2.19 mmol, 380.67 μL) into rb flask. Then added dry acetonitrile (50 mL). Stirred well to dissolve and then HBTU (217.57 mg, 573.70 μmol) to preactivate acid. Let stir for ˜5 min, then added CPG. Capped and put on mechanical shaker overnight. Next day filtered CPG, washed with ACN, then MeOH, then ACN, then diethyl ether. Dried for ˜5 minutes, then transferred back to rb flask for capping. Added 30% acetic anhydride in pyridine (50 ml total) and 1% TEA. Capped and put back on mechanical shaker for 3 hours. After 3 hours took off and washed CPG as follows: 10% H₂O/THF, then MeOH, then 10% H₂O/THF, then MeOH, then ACN, the ether (˜250 mL each solvent for washing). Transferred to rb flask and dried CPG in high vacuum overnight.

Checking the Loading: Weighted out 38 mg and loaded into 250 ml volumetric flask. Then added 0.1M toluene-p-sulfonic acid in ACN up to measure line. Sonicated and settled for 1 hour. Checked loading by spectrophotometer and beers law. Measured solution into UV cuvette and measured UV absorbance at 411 nm. Check worksheet for raw data. Calculated loading using beers law=[250 (mL)×(absorbance A)×35.5 (extinction coefficient of DMTr)]/weight of CPG (mg). Yield: 3.2 g, Loading: 107 μmol/g.

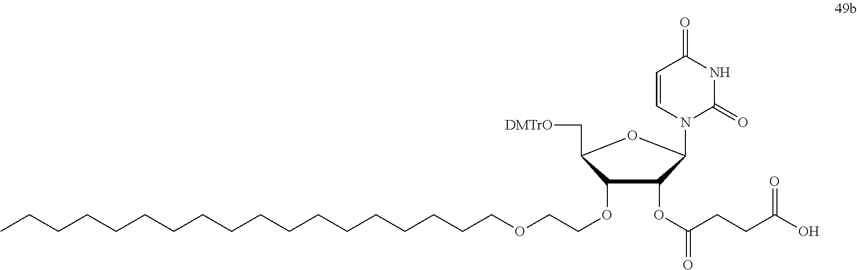

4-[(2R,5R)-5-[[bis(4-methoxyphenyl)-phenyl-methoxy]methyl]-2-(2,4-dioxopyrimidin-1-yl)-4-(2-octadecoxyethoxy)tetrahydrofuran-3-yl]oxy-4-oxo-butanoic acid 49b: To a solution of 45b (0.43 g, 0.510 mmol) in DCM (15 mL), 4-(dimethylamino)pyridine (189 mg, 1.53 mmol) and succinic anhydride (103 mg, 1.02 mmol) were added. The resulting mixture was stirred for 3 hrs at 22° C. All the volatile matters were evaporated to dryness and crude mass which was dissolved in EA (30 mL). The organic layer was washed with 10% NH₄Cl solution (3×20 mL), and brine (20 mL). The organic layer was separated, dried over anhydrous Na₂SO₄, filtered and the filtrate was evaporated to dryness. The crude mass thus obtained was purified by combiflash chromatography (Gradient: 0-5% Methanol in DCM) for afford 49b (0.4 g, 83% yield) as white solid. ¹H NMR (400 MHz, DMSO-d₆) δ 12.23 (s, 1H), 11.40 (d, J=2.2 Hz, 1H), 7.73 (d, J=8.1 Hz, 1H), 7.41-7.35 (m, 2H), 7.31 (dd, J=8.5, 6.7 Hz, 2H), 7.28-7.19 (m, 5H), 6.92-6.81 (m, 4H), 5.83 (d, J=3.3 Hz, 1H), 5.45 (dd, J=5.3, 3.4 Hz, 1H), 5.36 (dd, J=8.1, 2.2 Hz, 1H), 4.31 (dd, J=6.9, 5.3 Hz, 1H), 4.01 (dt, J=6.9, 3.5 Hz, 1H), 3.74 (s, 6H), 3.62-3.45 (m, 2H), 3.37 (t, J=4.7 Hz, 2H), 3.34-3.20 (m, 5H), 2.59 (ddd, J=5.8, 3.5, 2.0 Hz, 2H), 2.50 (dt, J=3.9, 1.9 Hz, 2H), 1.39 (t, J=6.6 Hz, 2H), 1.21 (d, J=11.2 Hz, 25H), 0.90-0.79 (m, 3H) ppm. ¹³C NMR (101 MHz, DMSO-d₆) δ 173.09, 171.14, 162.99, 158.14, 158.12, 150.13, 144.54, 140.70, 135.22, 135.08, 129.74, 129.70, 127.86, 127.68, 126.75, 113.21, 101.65, 87.61, 85.95, 80.74, 75.87, 73.37, 70.30, 69.90, 69.35, 62.07, 55.01, 31.28, 29.14, 29.00, 28.98, 28.85, 28.69, 28.59, 25.58, 22.08, 13.93 ppm. HRMS calc. for C₅₄H₇₄N₂O₁₂Na [M+Na]⁺ 965.5139, found 965.5126.

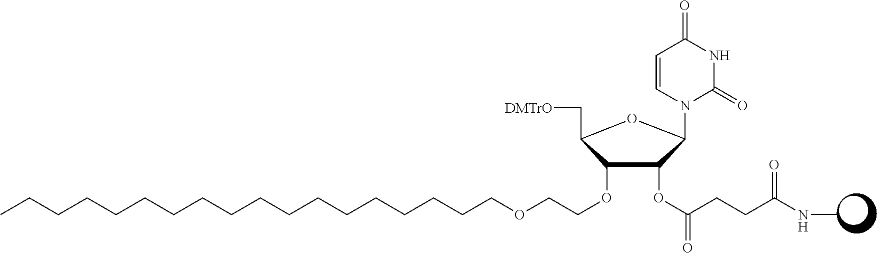

CPG from 49b: Added 4-[(2R,5R)-5-[[bis(4-methoxyphenyl)-phenyl-methoxy]methyl]-2-(2,4-dioxopyrimidin-1-yl)-4-(2-octadecoxyethoxy)tetrahydrofuran-3-yl]oxy-4-oxo-butanoic acid (0.4 g, 424.10 mol) and N-ethyl-N-isopropyl-propan-2-amine (219.24 mg, 1.70 mmol, 295.48 μL) into rb flask. Then added dry acetonitrile (50 mL). Stirred well to dissolve and then HBTU (168.88 mg, 445.31 μmol) to preactivate acid. Let stir for ˜5 min, then added CPG. Capped and put on mechanical shaker overnight. Next day filtered CPG, washed with ACN, then MeOH, then ACN, then diethyl ether. Dried for ˜5 minutes, then transferred back to rb flask for capping. Added 30% acetic anhydride in pyridine (50 ml total) and 1% TEA. Capped and put back on mechanical shaker for 3 hours. After 3 hours took off and washed CPG as follows: 10% H₂O/THF, then MeOH, then 10% H₂O/THF, then MeOH, then ACN, the ether (˜250 mL each solvent for washing). Transferred to rb flask and dried CPG in high vacuum overnight.

Checking the Loading: Weighted out 38 mg and loaded into 250 ml volumetric flask. Then added 0.1M toluene-p-sulfonic acid in ACN up to measure line. Sonicated and settled for 1 hour. Checked loading by spectrophotometer and beers law. Measured solution into UV cuvette and measured UV absorbance at 411 nm. Check worksheet for raw data. Calculated loading using beers law=[250 (mL)×(absorbance A)×35.5 (extinction coefficient of DMTr)]/weight of CPG (mg). Yield: 4 g, Loading: 97.7 μmol/g.

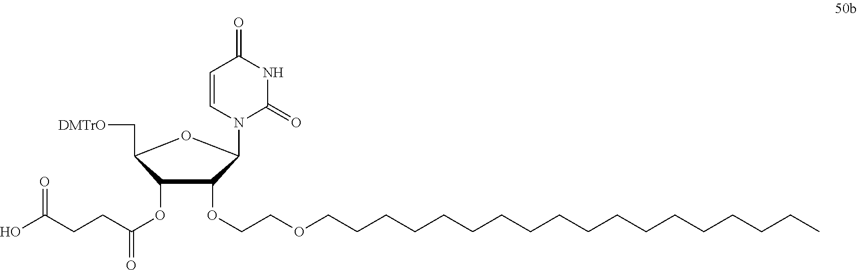

4-[(2R,5R)-2-[[bis(4-methoxyphenyl)-phenyl-methoxy]methyl]-5-(2,4-dioxopyrimidin-1-yl)-4-(2-octadecoxyethoxy)tetrahydrofuran-3-yl]oxy-4-oxo-butanoic acid 50b: To a solution of 46b (0.42 g, 498 mmol) in DCM (15 mL), 4-(dimethylamino)pyridine (184 mg, 1.49 mmol) and succinic anhydride (101 mg, 0.996 mmol) were added. The resulting mixture was stirred for 3 hrs at 22° C. All the volatile matters were evaporated to dryness and crude mass which was dissolved in EA (30 mL). The organic layer was washed with 10% NH₄Cl solution (3×20 mL), and brine (20 mL). The organic layer was separated, dried over anhydrous Na₂SO₄, filtered and the filtrate was evaporated to dryness. The crude mass thus obtained was purified by combiflash chromatography (Gradient: 0-5% Methanol in DCM) for afford 50b (0.39 g, 83% yield) as white solid. ¹H NMR (400 MHz, DMSO-d₆) δ 12.24 (s, 1H), 11.44 (d, J=2.2 Hz, 1H), 7.69 (d, J=8.1 Hz, 1H), 7.40-7.28 (m, 4H), 7.24 (dq, J=9.5, 2.9, 2.1 Hz, 5H), 6.96-6.83 (m, 4H), 5.82 (d, J=5.1 Hz, 1H), 5.43 (dd, J=8.1, 2.2 Hz, 1H), 5.22 (t, J=5.3 Hz, 1H), 4.34 (t, J=5.4 Hz, 1H), 3.74 (s, 6H), 3.65-3.50 (m, 2H), 3.41 (t, J=5.0 Hz, 2H), 3.37-3.21 (m, 6H), 2.66-2.55 (m, 2H), 2.54-2.41 (m, 2H), 1.40 (q, J=6.7 Hz, 2H), 1.22 (d, J=7.4 Hz, 28H), 0.91-0.80 (m, 3H) ppm. ¹³C NMR (101 MHz, DMSO-d₆) δ 173.14, 171.41, 162.84, 158.16, 150.35, 144.49, 140.46, 135.19, 135.02, 129.72, 129.69, 128.89, 127.90, 127.65, 126.80, 113.26, 112.74, 102.01, 87.34, 86.08, 80.50, 78.79, 70.48, 70.36, 69.88, 69.26, 62.69, 55.02, 31.29, 29.12, 29.02, 29.00, 28.86, 28.70, 28.57, 25.56, 22.09, 13.93 ppm. HRMS calc. for C₅₄H₇₄N₂O₁₂Na [M+Na]⁺ 965.5139, found 965.5136.

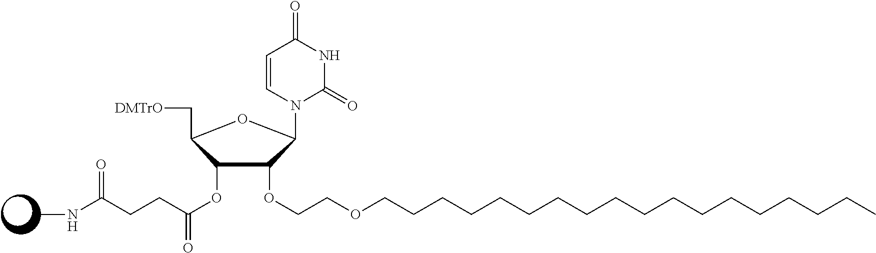

CPG from 50b: Added 4-[(2R,5R)-2-[[bis(4-methoxyphenyl)-phenyl-methoxy]methyl]-5-(2,4-dioxopyrimidin-1-yl)-4-(2-octadecoxyethoxy)tetrahydrofuran-3-yl]oxy-4-oxo-butanoic acid (0.39 g, 413.50 mol) and N-ethyl-N-isopropyl-propan-2-amine (213.76 mg, 1.65 mmol, 288.09 μL) into rb flask. Then added dry acetonitrile (50 mL). Stirred well to dissolve and then HBTU (164.66 mg, 434.17 μmol) to preactivate acid. Let stir for ˜5 min, then added CPG. Capped and put on mechanical shaker overnight. Next day filtered CPG, washed with ACN, then MeOH, then ACN, then diethyl ether. Dried for ˜5 minutes, then transferred back to rb flask for capping. Added 30% acetic anhydride in pyridine (50 ml total) and 1% TEA. Capped and put back on mechanical shaker for 3 hours. After 3 hours took off and washed CPG as follows: 10% H₂O/THF, then MeOH, then 10% H₂O/THF, then MeOH, then ACN, the ether (˜250 mL each solvent for washing). Transferred to rb flask and dried CPG in high vacuum overnight.

Checking the Loading: Weighted out 38 mg and loaded into 250 ml volumetric flask. Then added 0.1M toluene-p-sulfonic acid in ACN up to measure line. Sonicated and settled for 1 hour. Checked loading by spectrophotometer and beers law. Measured solution into UV cuvette and measured UV absorbance at 411 nm. Check worksheet for raw data. Calculated loading using beers law=[250 (mL)×(absorbance A)×35.5 (extinction coefficient of DMTr)]/weight of CPG (mg). Yield: 3.9 g, Loading: 95 μmol/g

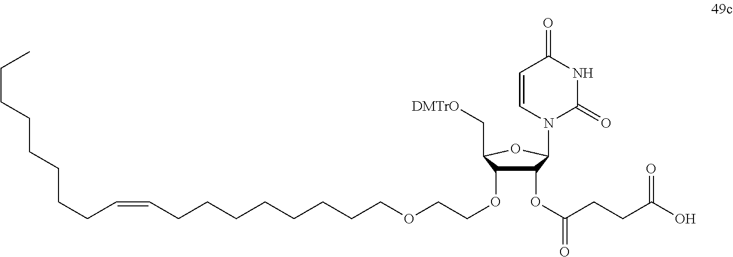

4-[(2R,5R)-5-[[bis(4-methoxyphenyl)-phenyl-methoxy]methyl]-2-(2,4-dioxopyrimidin-1-yl)-4-[2-[(Z)-octadec-9-enoxy]ethoxy]tetrahydrofuran-3-yl]oxy-4-oxo-butanoic acid 49c: To a solution of 45c (0.35 g, 416.13 μmol) in DCM (15 mL), 4-(dimethylamino)pyridine (154.06 mg, 1.25 mmol) and succinic anhydride (84.13 mg, 832.26 μmol) were added. The resulting mixture was stirred for 4 hrs at 22° C. The volatile matters were evaporated to dryness and crude mass which was dissolved in EA (30 mL). The organic layer was washed with 10% NH₄Cl solution (3×20 mL), and brine (20 mL). The organic layer was separated, dried over anhydrous Na₂SO₄, filtered and the filtrate was evaporated to dryness. The crude mass thus obtained was purified by combiflash chromatography (Gradient: 0-5% Methanol in DCM) for afford 49c (0.28 g, 72% yield) as white solid. ¹H NMR (400 MHz, Chloroform-d) δ 10.17 (s, 1H), 7.85 (d, J=8.1 Hz, 1H), 7.42-7.17 (m, 10H), 6.97-6.76 (m, 4H), 6.13 (d, J=3.9 Hz, 1H), 5.45 (dd, J=5.1, 4.1 Hz, 1H), 5.39-5.27 (m, 3H), 4.28 (t, J=5.5 Hz, 1H), 4.23-4.13 (m, 1H), 3.79 (s, 6H), 3.75-3.68 (m, 1H), 3.59-3.48 (m, 4H), 3.47-3.31 (m, 3H), 2.84-2.55 (m, 4H), 2.01 (q, J=6.7 Hz, 4H), 1.51 (q, J=6.9 Hz, 2H), 1.28 (d, J=11.0 Hz, 25H), 0.96-0.72 (m, 3H) ppm. ¹³C NMR (126 MHz, CDCl₃) δ 175.31, 171.05, 163.91, 158.88, 158.86, 150.81, 144.30, 140.11, 135.37, 135.18, 130.26, 130.24, 130.08, 129.96, 128.28, 128.18, 127.31, 113.47, 102.81, 87.26, 87.22, 82.27, 74.62, 71.75, 70.91, 70.22, 62.06, 55.39, 32.04, 29.91, 29.70, 29.66, 29.61, 29.47, 29.45, 29.43, 29.38, 29.19, 27.36, 26.17, 22.82, 14.25 ppm. HRMS calc. for C₅₄H₇₂N₂O₁₂Na [M+Na]⁺ 963.4983, found 963.4995.

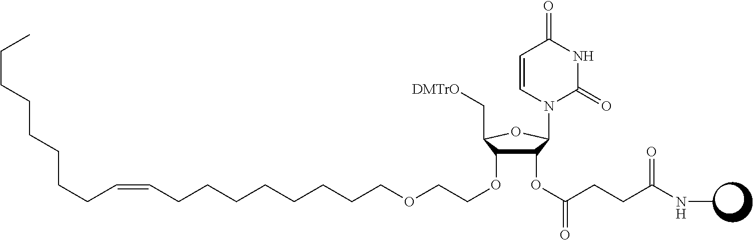

CPG from 49c: Added 4-[(2R,5R)-5-[[bis(4-methoxyphenyl)-phenyl-methoxy]methyl]-2-(2,4-dioxopyrimidin-1-yl)-4-[2-[(Z)-octadec-9-enoxy]ethoxy]tetrahydrofuran-3-yl]oxy-4-oxo-butanoic acid (0.26 g, 276.26 mol) and diisopropylethylamine (142.81 mg, 1.11 mmol, 192.47 μL) into rb flask. Then added dry acetonitrile (50 mL). Stirred well to dissolve and then HBTU (110.01 mg, 290.07 μmol) to preactivate acid. Let stir for ˜5 min, then added CPG. Capped and put on mechanical shaker overnight. Next day filtered CPG, washed with ACN, then MeOH, then ACN, then diethyl ether. Dried for ˜5 minutes, then transferred back to rb flask for capping. Added 30% acetic anhydride in pyridine (50 ml total) and 1% TEA. Capped and put back on mechanical shaker for 3 hours. After 3 hours took off and washed CPG as follows: 10% H₂O/THF, then MeOH, then 10% H₂O/THF, then MeOH, then ACN, the ether (˜250 mL each solvent for washing). Transferred to rb flask and dried CPG in high vacuum overnight.

Checking the Loading: Weighted out 50 mg and loaded into 250 ml volumetric flask. Then added 0.1M toluene-p-sulfonic acid in ACN up to measure line. Sonicated and settled for 1 hour. Checked loading by spectrophotometer and beers law. Measured solution into UV cuvette and measured UV absorbance at 411 nm. Check worksheet for raw data. Calculated loading using beers law=[250 (mL)×(absorbance A)×35.5 (extinction coefficient of DMTr)]/weight of CPG (mg). Yield: 2.54 g, Loading: 93 μmol/g.

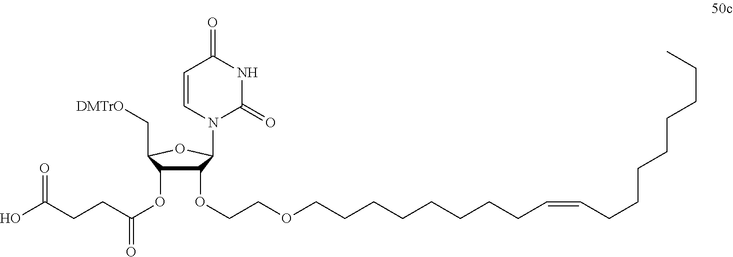

4-[(2R,5R)-2-[[bis(4-methoxyphenyl)-phenyl-methoxy]methyl]-5-(2,4-dioxopyrimidin-1-yl)-4-[2-[(Z)-octadec-9-enoxy]ethoxy]tetrahydrofuran-3-yl]oxy-4-oxo-butanoic acid 50c: To a solution of 46c (0.4 g, 475.58 μmol) in DCM (15 mL), 4-(dimethylamino)pyridine (176.07 mg, 1.43 mmol) and succinic anhydride (96.14 mg, 951.16 μmol) were added. The resulting mixture was stirred for 3 hrs at 22° C. The volatile matters were evaporated to dryness and crude mass which was dissolved in EA (30 mL). The organic layer was washed with 10% NH₄Cl solution (2×30 mL), and brine (20 mL). The organic layer was separated, dried over anhydrous Na₂SO₄, filtered and the filtrate was evaporated to dryness. The crude mass thus obtained was purified by combiflash chromatography (Gradient: 0-5% Methanol in DCM) for afford 50c (0.35 g, 78% yield) as white solid. ¹H NMR (400 MHz, Chloroform-d) δ 10.17-9.67 (m, 1H), 8.07 (d, J=8.1 Hz, 1H), 7.37-7.19 (m, 9H), 6.88-6.73 (m, 4H), 5.92 (d, J=2.0 Hz, 1H), 5.44-5.27 (m, 3H), 5.23 (dd, J=8.0, 5.0 Hz, 1H), 4.34 (dt, J=8.0, 2.2 Hz, 1H), 4.16 (dd, J=4.9, 2.1 Hz, 1H), 3.92 (ddd, J=11.2, 5.7, 2.8 Hz, 1H), 3.79 (d, J=0.8 Hz, 7H), 3.69-3.56 (m, 3H), 3.50-3.36 (m, 3H), 2.90-2.39 (m, 4H), 2.00 (dq, J=5.5, 3.7 Hz, 4H), 1.56 (t, J=7.0 Hz, 2H), 1.28 (d, J=13.6 Hz, 23H), 1.01-0.66 (m, 3H) ppm. ¹³C NMR (126 MHz, CDCl₃) δ 175.53, 171.53, 164.57, 158.88, 158.86, 149.93, 144.30, 140.52, 135.16, 135.09, 130.33, 130.27, 130.06, 129.96, 128.24, 128.17, 127.32, 113.46, 113.44, 101.85, 88.41, 87.45, 81.19, 80.55, 71.63, 70.84, 70.30, 69.67, 60.90, 55.39, 32.04, 29.91, 29.68, 29.66, 29.61, 29.58, 29.46, 29.44, 29.17, 29.03, 27.36, 26.16, 22.82, 14.25 ppm. HRMS calc. for C₅₄H₇₂N₂O₁₂Na [M+Na]⁺ 963.4983, found 963.4974.

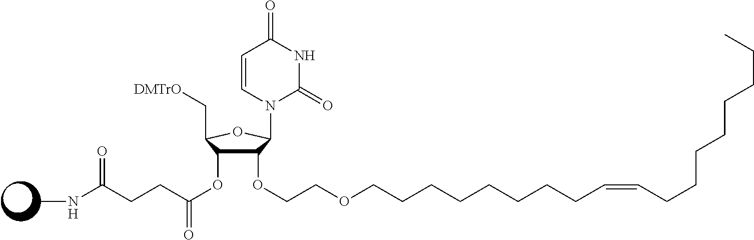

CPG from 50c: Added 4-[(2R,5R)-2-[[bis(4-methoxyphenyl)-phenyl-methoxy]methyl]-5-(2,4-dioxopyrimidin-1-yl)-4-[2-[(Z)-octadec-9-enoxy]ethoxy]tetrahydrofuran-3-yl]oxy-4-oxo-butanoic acid (0.34 g, 361.26 mol) and diisopropylethylamine (186.76 mg, 1.45 mmol, 251.69 μL) into rb flask. Then added dry acetonitrile (50 mL). Stirred well to dissolve and then HBTU (143.85 mg, 379.32 μmol) to preactivate acid. Let stir for ˜5 min, then added CPG. Capped and put on mechanical shaker overnight. Next day filtered CPG, washed with ACN, then MeOH, then ACN, then diethyl ether. Dried for ˜5 minutes, then transferred back to rb flask for capping. Added 30% acetic anhydride in pyridine (50 ml total) and 1% TEA. Capped and put back on mechanical shaker for 3 hours. After 3 hours took off and washed CPG as follows: 10% H₂O/THF, then MeOH, then 10% H₂O/THF, then MeOH, then ACN, the ether (˜250 mL each solvent for washing). Transferred to rb flask and dried CPG in high vacuum overnight.

Checking the Loading: Weighted out 33.3 mg and loaded into 250 ml volumetric flask. Then added 0.1M toluene-p-sulfonic acid in ACN up to measure line. Sonicated and settled for 1 hour. Checked loading by spectrophotometer and beers law. Measured solution into UV cuvette and measured UV absorbance at 411 nm. Check worksheet for raw data. Calculated loading using beers law=[250 (mL)×(absorbance A)×35.5 (extinction coefficient of DMTr)]/weight of CPG (mg). Yield: 3.27 g, Loading: 87 μmol/g.

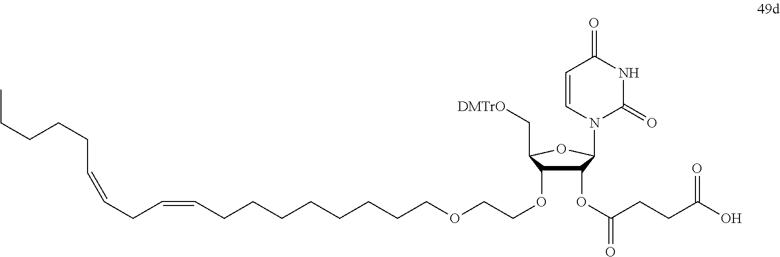

4-[(2R,5R)-5-[[bis(4-methoxyphenyl)-phenyl-methoxy]methyl]-2-(2,4-dioxopyrimidin-1-yl)-4-[2-[(9Z,12Z)-octadeca-9,12-dienoxy]ethoxy]tetrahydrofuran-3-yl]oxy-4-oxo-butanoic acid 49d: To a solution of 45d (0.3 g, 357.54 μmol) in DCM (15 mL), 4-(dimethylamino)pyridine (132.37 mg, 1.07 mmol) and succinic anhydride (72.28 mg, 715.08 mol) were added. The resulting mixture was stirred for 3 hrs at 22° C. The volatile matters were evaporated to dryness and crude mass which was dissolved in EA (30 mL). The organic layer was washed with 10% NH₄Cl solution (2×30 mL), and brine (20 mL). The organic layer was separated, dried over anhydrous Na₂SO₄, filtered and the filtrate was evaporated to dryness. The crude mass thus obtained was purified by combiflash chromatography (Gradient: 0-5% Methanol in DCM) for afford 49d (0.30 g, 88% yield) as white solid. ¹H NMR (400 MHz, Chloroform-d) δ 10.04 (s, 1H), 7.85 (d, J=8.2 Hz, 1H), 7.42-7.17 (m, 10H), 6.91-6.69 (m, 4H), 6.13 (d, J=4.0 Hz, 1H), 5.45 (dd, J=5.1, 4.0 Hz, 1H), 5.43-5.21 (m, 5H), 4.28 (t, J=5.4 Hz, 1H), 4.18 (dt, J=5.5, 2.5 Hz, 1H), 3.79 (s, 6H), 3.72 (ddd, J=10.2, 4.7, 3.5 Hz, 1H), 3.57-3.47 (m, 4H), 3.44-3.32 (m, 3H), 2.87-2.54 (m, 6H), 2.04 (qd, J=6.8, 2.3 Hz, 4H), 1.51 (q, J=6.9 Hz, 2H), 1.40-1.16 (m, 17H), 0.98-0.68 (m, 3H) ppm. ¹³C NMR (126 MHz, CDCl₃) δ 175.19, 171.04, 163.84, 158.89, 158.87, 150.76, 144.30, 140.11, 135.37, 135.18, 130.35, 130.26, 130.24, 128.29, 128.19, 128.13, 128.07, 127.33, 113.47, 102.81, 87.28, 87.21, 82.26, 74.61, 71.76, 70.89, 70.24, 62.07, 55.40, 31.67, 29.82, 29.69, 29.66, 29.60, 29.49, 29.44, 29.19, 27.39, 27.35, 26.17, 25.78, 22.72, 14.22 ppm. HRMS calc. for C₅₄H₇₀N₂O₁₂Na [M+Na]⁺ 961.4826, found 961.4846.

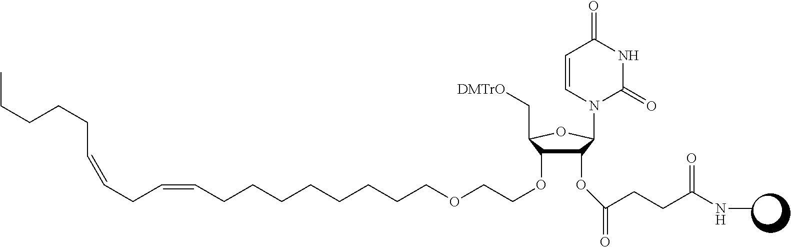

CPG from 49d: Added 4-[(2R,5R)-5-[[bis(4-methoxyphenyl)-phenyl-methoxy]methyl]-2-(2,4-dioxopyrimidin-1-yl)-4-[2-[(9Z,12Z)-octadeca-9,12-dienoxy]ethoxy]tetrahydrofuran-3-yl]oxy-4-oxo-butanoic acid (0.2 g, 212.96 mol) and N-ethyl-N-isopropyl-propan-2-amine (110.09 mg, 851.85 μmol, 148.37 μL) into rb flask. Then added dry acetonitrile (50 mL). Stirred well to dissolve and then HBTU (84.80 mg, 223.61 μmol) to preactivate acid. Let stir for ˜5 min, then added CPG. Capped and put on mechanical shaker overnight. Next day filtered CPG, washed with ACN, then MeOH, then ACN, then diethyl ether. Dried for ˜5 minutes, then transferred back to rb flask for capping. Added 30% acetic anhydride in pyridine (50 ml total) and 1% TEA. Capped and put back on mechanical shaker for 3 hours. After 3 hours took off and washed CPG as follows: 10% H₂O/THF, then MeOH, then 10% H₂O/THF, then MeOH, then ACN, the ether (˜250 mL each solvent for washing). Transferred to rb flask and dried CPG in high vacuum overnight.

Checking the Loading: Weighted out 52.9 mg and loaded into 250 ml volumetric flask. Then added 0.1M toluene-p-sulfonic acid in ACN up to measure line. Sonicated and settled for 1 hour. Checked loading by spectrophotometer and beers law. Measured solution into UV cuvette and measured UV absorbance at 411 nm. Check worksheet for raw data. Calculated loading using beers law=[250 (mL)×(absorbance A)×35.5 (extinction coefficient of DMTr)]/weight of CPG (mg). Yield: 1.94 g, Loading: 98 μmol/g.

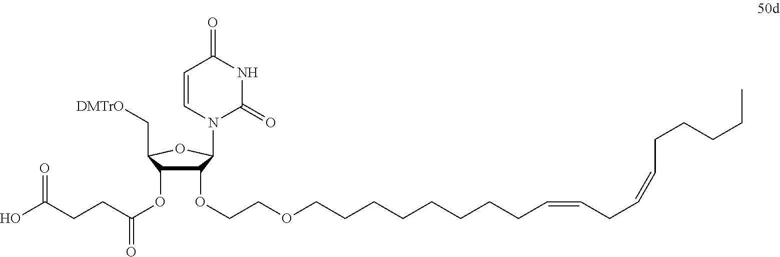

4-[(2R,5R)-2-[[bis(4-methoxyphenyl)-phenyl-methoxy]methyl]-5-(2,4-dioxopyrimidin-1-yl)-4-[2-[(9Z,12Z)-octadeca-9,12-dienoxy]ethoxy]tetrahydrofuran-3-yl]oxy-4-oxo-butanoic acid 50d: To a solution of 46d (0.3 g, 357.54 μmol) in DCM (15 mL), 4-(dimethylamino)pyridine (132.37 mg, 1.07 mmol) and succinic anhydride (72.28 mg, 715.08 mol) were added. The resulting mixture was stirred for 3 hrs at 22° C. The volatile matters were evaporated to dryness and crude mass which was dissolved in EA (30 mL). The organic layer was washed with 10% NH₄Cl solution (3×20 mL), and brine (20 mL). The organic layer was separated, dried over anhydrous Na₂SO₄, filtered and the filtrate was evaporated to dryness. The crude mass thus obtained was purified by combiflash chromatography (Gradient: 0-5% Methanol in DCM) for afford 50d (0.29 g, 87% yield) as white solid. ¹H NMR (400 MHz, Chloroform-d) δ 10.22 (d, J=6.4 Hz, 1H), 8.03 (dd, J=8.1, 1.8 Hz, 1H), 7.52-7.10 (m, 10H), 6.91-6.54 (m, 4H), 5.95 (d, J=2.4 Hz, 1H), 5.42-5.28 (m, 6H), 5.23 (dd, J=7.4, 5.0 Hz, 1H), 4.33 (dd, J=7.6, 2.1 Hz, 1H), 4.20 (dd, J=5.1, 2.5 Hz, 1H), 3.98-3.85 (m, 1H), 3.82-3.68 (m, 8H), 3.68-3.54 (m, 3H), 3.49-3.37 (m, 3H), 2.88-2.52 (m, 6H), 2.15-1.87 (m, 5H), 1.54 (q, J=6.9 Hz, 2H), 1.44-1.15 (m, 20H), 0.88 (t, J=6.7 Hz, 3H) ppm. ¹³C NMR (126 MHz, Chloroform-d) δ 175.99, 171.51, 164.65, 158.82, 158.81, 150.10, 144.26, 140.50, 135.12, 135.05, 130.26, 130.21, 130.20, 128.19, 128.12, 128.05, 128.02, 127.26, 113.40, 101.92, 88.23, 87.40, 81.10, 80.67, 71.55, 70.80, 70.15, 69.89, 61.07, 55.33, 31.60, 29.80, 29.76, 29.61, 29.57, 29.55, 29.43, 29.38, 29.00, 28.96, 27.33, 27.28, 26.11, 25.72, 22.65, 14.16 ppm. HRMS calc. for C₅₄H₇₀N₂O₁₂Na [M+Na]⁺ 961.4826, found 961.4858.

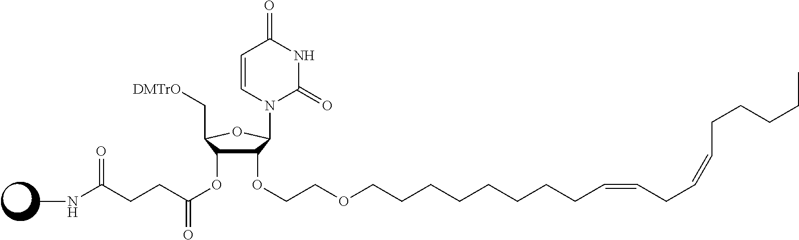

CPG from 50d: Added 4-[(2R,5R)-2-[[bis(4-methoxyphenyl)-phenyl-methoxy]methyl]-5-(2,4-dioxopyrimidin-1-yl)-4-[2-[(9Z,12Z)-octadeca-9,12-dienoxy]ethoxy]tetrahydrofuran-3-yl]oxy-4-oxo-butanoic acid (0.22 g, 234.26 mol) and N-ethyl-N-isopropyl-propan-2-amine (121.10 mg, 937.03 μmol, 163.21 μL) into rb flask. Then added dry acetonitrile (50 mL). Stirred well to dissolve and then HBTU (93.28 mg, 245.97 μmol) to preactivate acid. Let stir for ˜5 min, then added CPG. Capped and put on mechanical shaker overnight. Next day filtered CPG, washed with ACN, then MeOH, then ACN, then diethyl ether. Dried for ˜5 minutes, then transferred back to rb flask for capping. Added 30% acetic anhydride in pyridine (50 mL total) and 1% TEA. Capped and put back on mechanical shaker for 3 hours. After 3 hours took off and washed CPG as follows: 10% H₂O/THF, then MeOH, then 10% H₂O/THF, then MeOH, then ACN, the ether (˜250 mL each solvent for washing). Transferred to rb flask and dried CPG in high vacuum overnight.

Checking the Loading: Weighed and loaded into 250 ml volumetric flask. Then added 0.1M toluene-p-sulfonic acid in ACN up to measure line. Sonicated and settled for 1 hour. Checked loading by spectrophotometer and beers law. Measured solution into UV cuvette and measured UV absorbance at 411 nm. Check worksheet for raw data. Calculated loading using beers law=[250 (mL)×(absorbance A)×35.5 (extinction coefficient of DMTr)]/weight of CPG (mg). Yield: 1.97 g, Loading: 93 μmol/g.

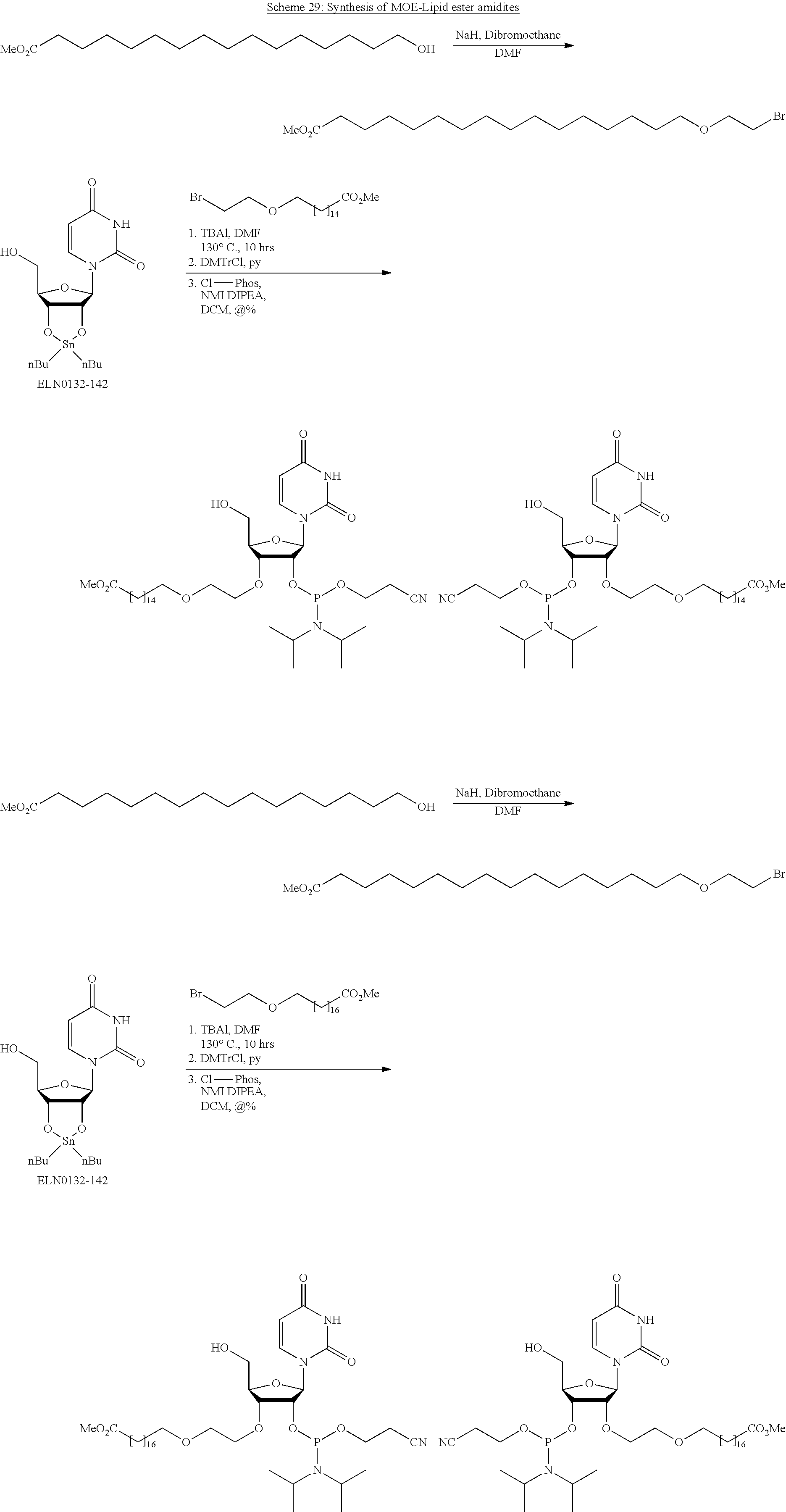

### REFERENCES

(1) Wagner, D.; Verheyden, J. P. H.; Moffatt, J. G. J Org. Chem. 1974, 39, 24.
(2) Moore, J. E.; McCoy, T. M.; Sokolova, A. V.; de Campo, L.; Pearson, G. R.; Wilkinson, B. L.; Tabor, R. F. J Colloid Interface Sci 2019, 547, 275.
(3) Noe, C. R.; Winkler, J.; Urban, E.; Gilbert, M.; Haberhauer, G.; Brunar, H. Nucleosides, Nucleotides & Nucleic Acids 2005, 24, 1167.
(4) Singh, R. S.; Mukherjee, K.; Banerjee, R.; Chaudhuri, A.; Hait, S. K.; Moulik, S. P.; Ramadas, Y.; Vijayalakshmi, A.; Rao, N. M. Chemistry—A European Journal 2002, 8, 900.

### Example 3: Synthesis of MOE-C16 and C18 Flip Lipid Ester Amidites

MOE-C16 and C18 flip lipid ester amidites are synthesized according to the synthesis shown in Scheme 30.

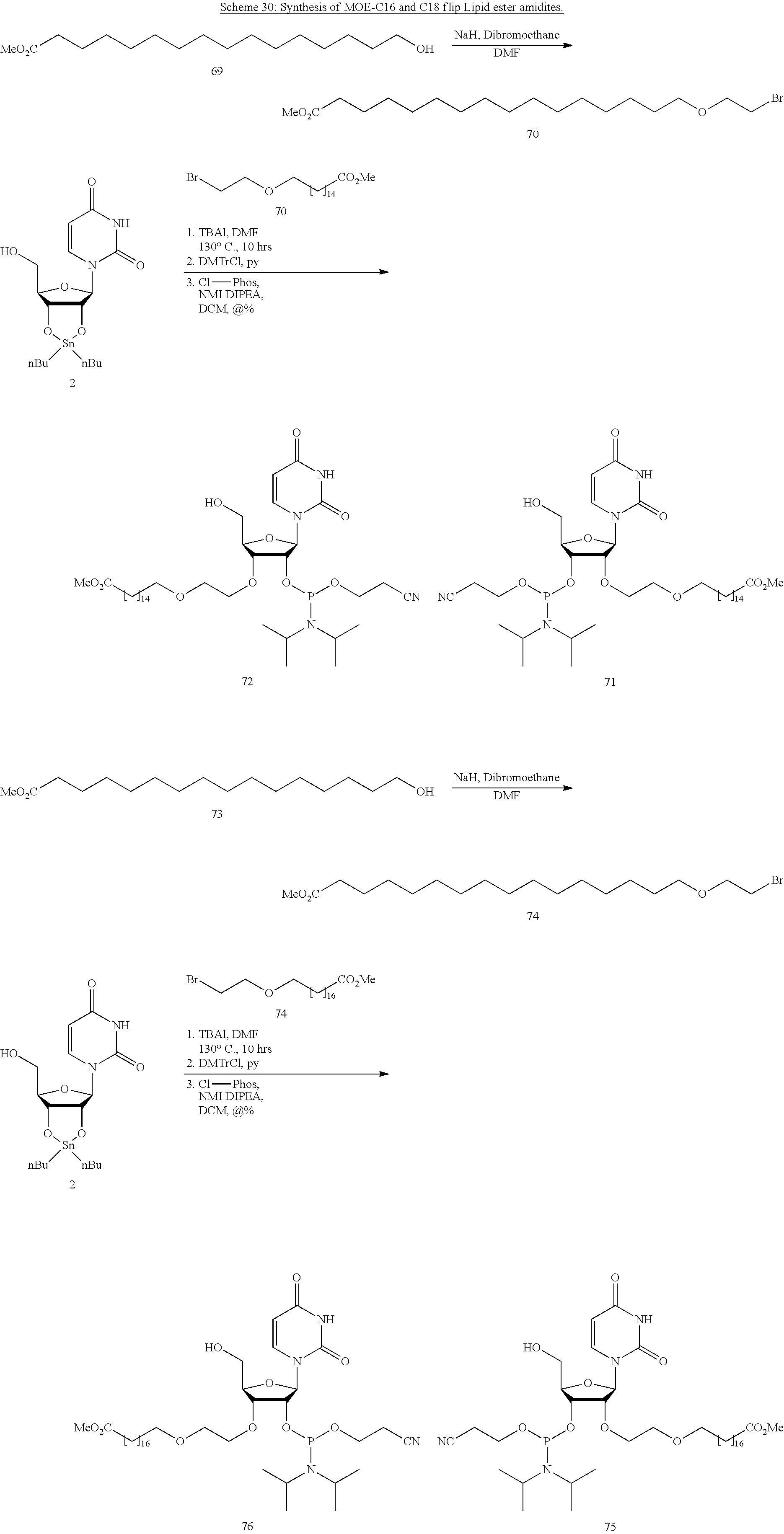

### Example 4: Some exemplary lipophilic Modifications through 2′- and 3′-alkyl, MOE and NMA functionalities

Ligand: Tocopherol (301-306)
B=all nucleobases

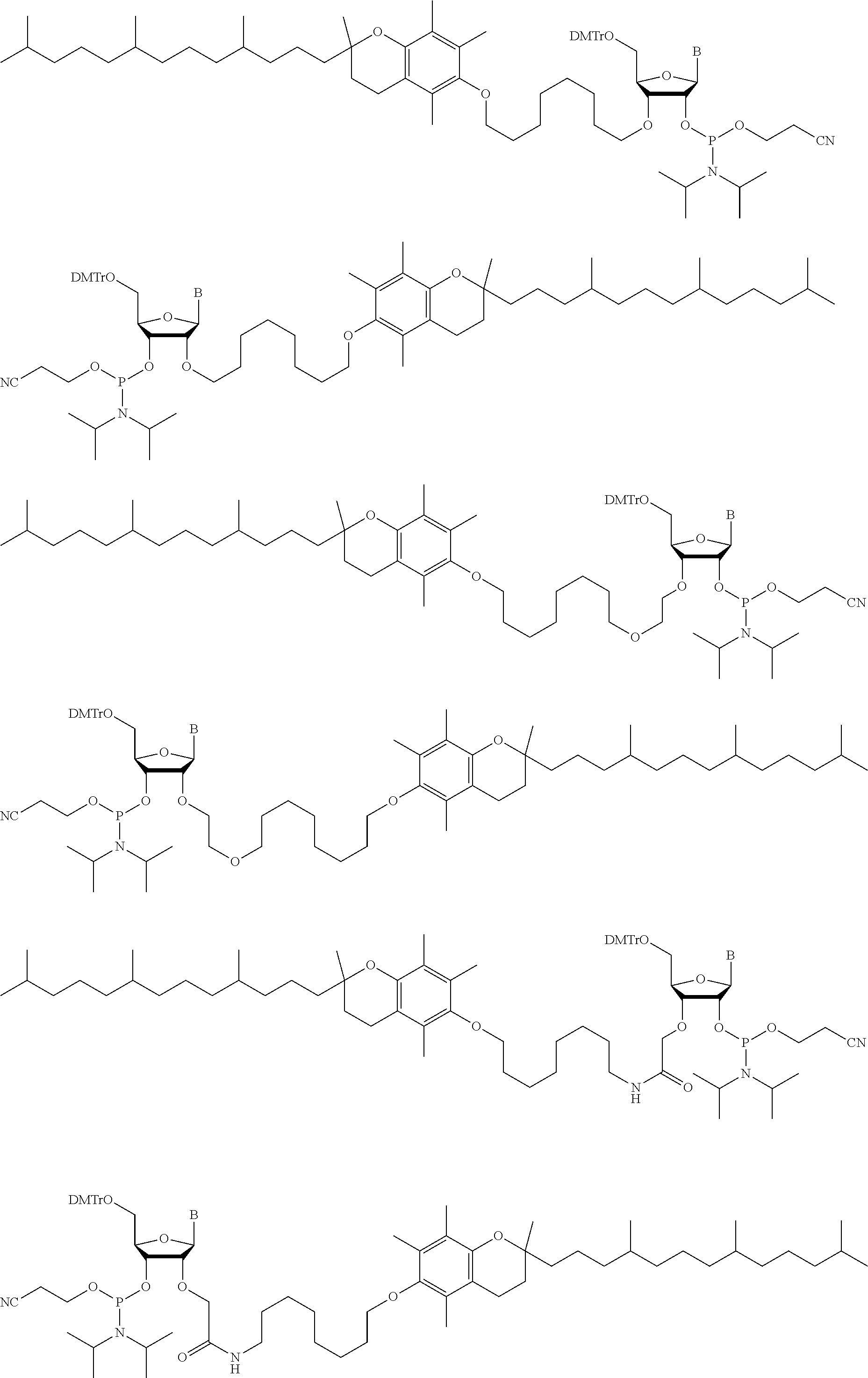

Ligand: Lipoic acid (307-312)
B=all nucleobases

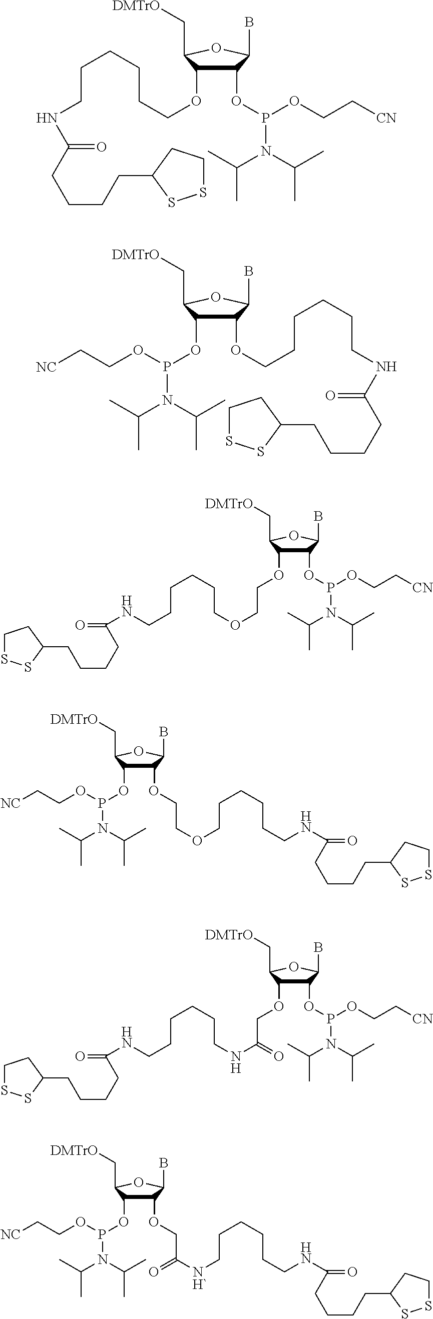

Ligand: Capsaicin (313-318)
B=all nucleobases

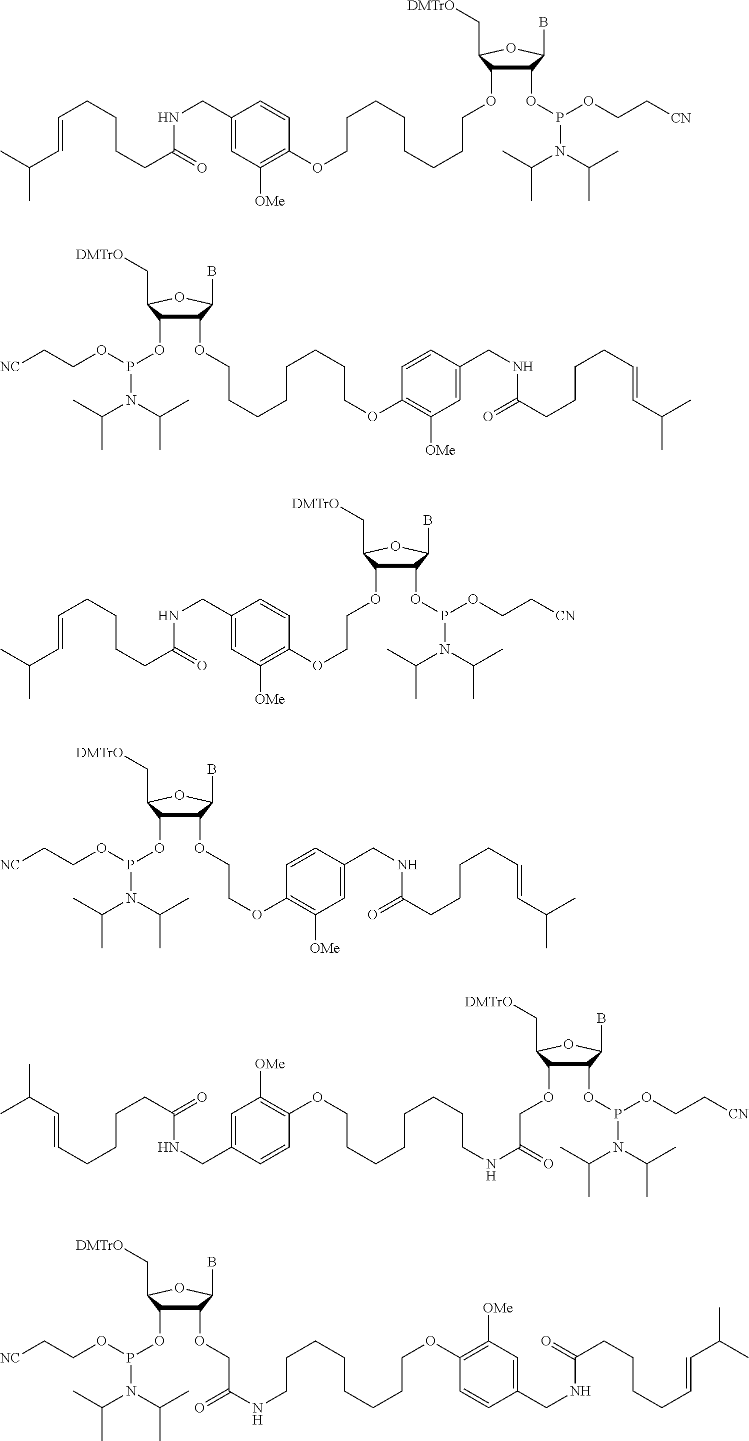

Ligand: sphingolipids (319-324)
B=all nucleobases

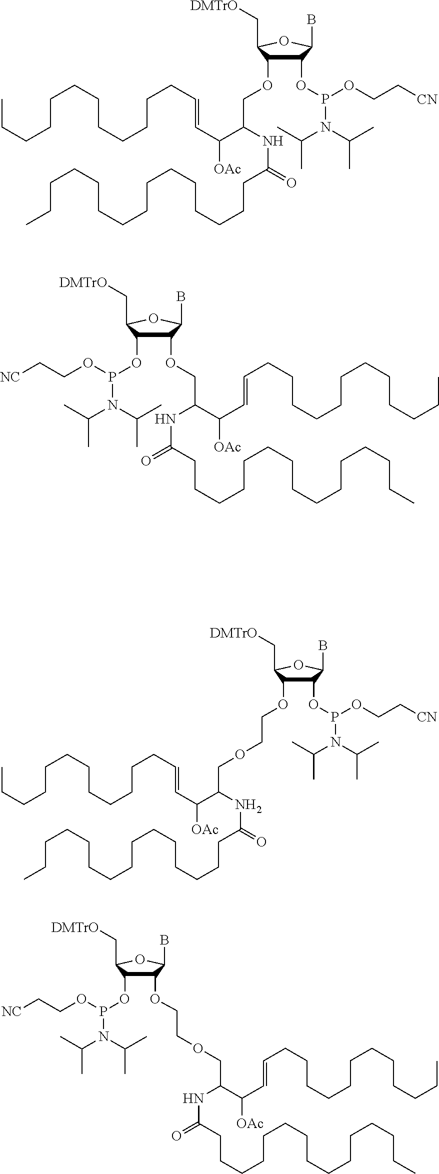

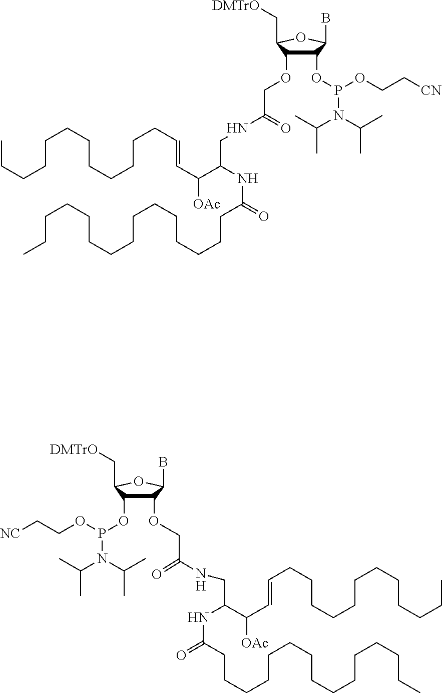

Ligand: Biotin (325-330)
B=all nucleobases

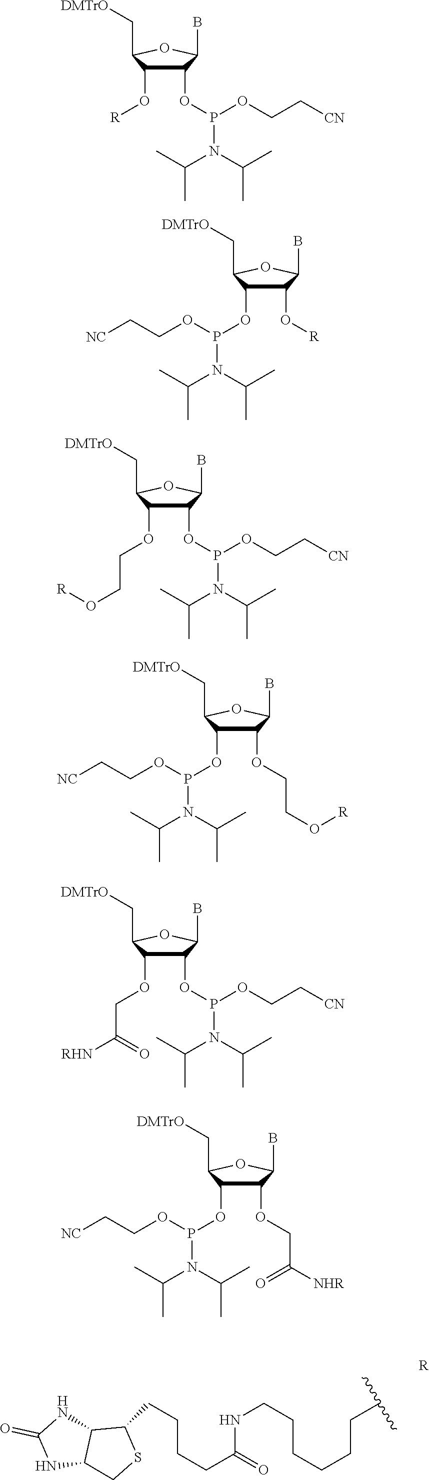

Ligand: Chloroalkane (331-336)
B=all nucleobases

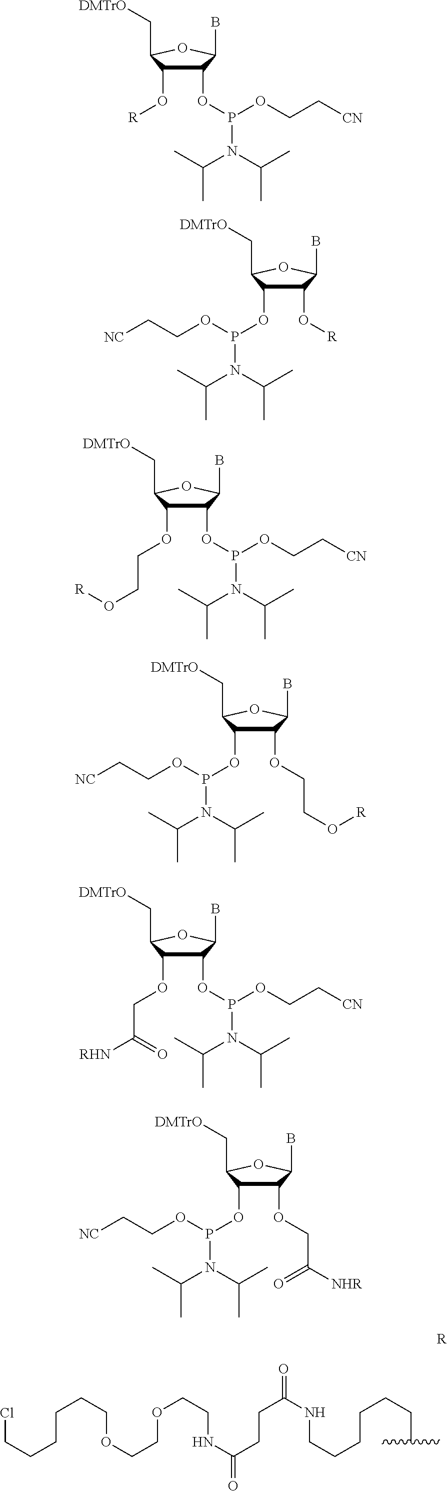

### Example 5

Double-stranded RNA molecules and their sequences used in this example are shown in Table 4.

Oligonucleotide Synthesis: Oligonucleotides were synthesized on either an ABI-394 at 1-μmol scale using universal supports or a K&A H-8-SE at 40-μmol scale using universal supports. A solution of 0.25 M 5-(S-ethylthio)-1H-tetrazole in acetonitrile (CH₃CN) was used as the activator. The solutions of commercially available phosphoramidites and synthesized phosphoramidities were used at 0.15 M in anhydrous CH₃CN or CH₂Cl₂. The oxidizing reagent was 0.02 M 12 in THF/pyridine/H₂O. N,N-Dimethyl-N′-(3-thioxo-3H-1,2,4-dithiazol-5-yl)methanimidamide (DDTT), 0.1 M in pyridine, was used as the sulfurizing reagent. The detritylation reagent was 3% dichloroacetic acid in CH₂Cl₂. Waiting time for coupling, capping, oxidation, and sulfurization step are 450s, 25s, 80s and 300s respectively. After completion of the automated synthesis, the oligonucleotide was manually released from support and deprotected using 28-30% ammonium hydroxide solution at 60° C. for 5 h. After filtration through a 0.45-μm nylon filter, oligonucleotides were purified by ion exchange and/or reverse phase column chromatography. For ion exchange, preparative HPLC custom packed with TSKGel SuperQ-5PW(20) (Sigma) using an appropriate gradient of mobile phase (buffer A: 20 mM sodium phosphate, 15% CH₃CN, pH 8.5; buffer B: 1 M NaBr, 20 mM sodium phosphate, 15% CH₃CN, pH 8.5) and desalted using size-exclusion chromatography using a custom packed with Sephadex G25 (GE Healthcare) and water as an eluent. For reverse phase, preparative HPLC (prep RP-HPLC, Agilent, ZORBAX 300SB—C₁₈ 5 μm 9.4×250 mm) using an appropriate gradient of mobile phase (buffer A: 50 mM TEAA, 3% CH₃CN; buffer B 50 mM TEAA, 80% CH₃CN) and desalted using size-exclusion chromatography using a custom packed with Sephadex G25 (GE Healthcare) and water as an eluent. Triethyl ammonium cation was displaced by Na with an excess of 0.1M AcONa solution, and a second desalting process. Oligonucleotides were then quantified by measuring the absorbance at 260 nm. Extinction coefficients were calculated using the following extinction coefficients for each residue: A, 13.86; T/U, 7.92; C, 6.57; and G, 10.53 M⁻¹ cm⁻¹. The purity and identity of modified ONs were verified by analytical reRP-HPLC chromatography and mass spectrometry, respectively. The activity assay:

### SOD-1:

Mouse ICV: Mouse ICV was performed as described previously (Brown et. al. Nat. Biotech. 2022, 40, 1500-1508) with modifications. The siRNAs formulated at 100 g in artificial cerebrospinal fluid (aCSF) were administered as 10 μL ICV injections in single dose, to female C57/BL6 mice (aged 6-8 weeks; N=4). The tissues from right hemisphere were collected on D15. The results of the SOD1 mRNA knockdown in brain hemisphere in mice on D15 were analyzed. The controls were aCSF without siRNAs administrations.

Rat IT: Rat IT was performed as described previously (Brown et. al. Nat. Biotech. 2022, 40, 1500-1508). Briefly, the siRNAs formulated at 0.9 mg in artificial cerebrospinal fluid (aCSF) were administered as 30 μL direct stick IT injections in a single dose, to female Sprague Dawley rat (aged 6-8 weeks; N=3). The tissues from CNS (striatum, frontal cortex, hippocampus, lumbar spinal cord were collected on D15. The results of the SOD1 mRNA knockdown in various CNS tissues in rat on D15 were analyzed. The controls were aCSF without siRNAs administrations.

ocTTR:

One eye of each mouse was administered a single 2.5 ug dose of a dsRNA agent or PBS (as a control) via intravitreal injection. Efficacy of treatment was evaluated by measurement of TTR mRNA levels in the eye at 14 days post-dose via qPCR. The mRNA level was calculated for each group and normalized to untreated tissue sample to give relative TTR mRNA as a % message remaining compared to the untreated tissue. TTR gene silencing was studied by qPCR in mouse eye following intravitreal administration of a single 2.5 ug dose of siRNA duplexes, with the mouse sacrificed on day 14, and the results were compared to PBS dosed control.

TABLE 4
Sequences used in this study

| Duplex | | | |
|---|---|---|---|
| ID | Target | Snese trand (5′->3′) | Antisense strand (5′->3′) |
| | SOD1 | CAUUUUAAUCCUCACUCUAAA (SEQ ID NO: 8) | UUUAGAGUGAGGAUUAAAAUGAG (SEQ ID NO: 9) |
| AD- | SOD1 | csasuuu(Uhd)AfaUfCfCfucacucuasasa | VPusUfsuagAfgUfGfaggaUfuAfaaaugsasg |
| 401824 | | (SEQ ID NO: 10) | (SEQ ID NO: 11) |
| AD- | SOD1 | csasuuuY179AfaUfCfCfucacucuasasa | VPusUfsuagAfgUfGfaggaUfuAfaaaugsasg |
| 1620931 | | (SEQ ID NO: 12) | (SEQ ID NO: 13) |
| AD- | SOD1 | csasuuuY180AfaUfCfCfucacucuasasa | VPusUfsuagAfgUfGfaggaUfuAfaaaugsasg |
| 1620932 | | (SEQ ID NO: 14) | (SEQ ID NO: 15) |
| AD- | SOD1 | csasuuuY182AfaUfCfCfucacucuasasa | VPusUfsuagAfgUfGfaggaUfuAfaaaugsasg |
| 1620933 | | (SEQ ID NO: 16) | (SEQ ID NO: 17) |
| AD- | SOD1 | csasuuuY184AfaUfCfCfucacucuasasa | VPusUfsuagAfgUfGfaggaUfuAfaaaugsasg |
| 1620934 | | (SEQ ID NO: 18) | (SEQ ID NO: 19) |
| AD- | SOD1 | csasuuuY208 AfaUfCfCfucacucuasasa | VPusUfsuagAfgUfGfaggaUfuAfaaaugsasg |
| 1620936 | | (SEQ ID NO: 20) | (SEQ ID NO: 21) |
| AD- | SOD1 | csasuuuY210AfaUfCfCfucacucuasasa | VPusUfsuagAfgUfGfaggaUfuAfaaaugsasg |
| 1620937 | | (SEQ ID NO: 22) | (SEQ ID NO: 23) |
| AD- | SOD1 | csasuuuY209AfaUfCfCfucacucuasasa | VPusUfsuagAfgUfGfaggaUfuAfaaaugsasg |
| 1640308 | | (SEQ ID NO: 24) | (SEQ ID NO: 25) |
| AD- | SOD1 | csasuuuY250AfaUfCfCfucacucuasasa | VPusUfsuagAfgUfGfaggaUfuAfaaaugsasg |
| 1962191 | | (SEQ ID NO: 26) | (SEQ ID NO: 27) |
| AD- | SOD1 | csasuuuY270AfaUfCfCfucacucuasasa | VPusUfsuagAfgUfGfaggaUfuAfaaaugsasg |
| 1962192 | | (SEQ ID NO: 28) | (SEQ ID NO: 29) |
| | TTR | AACAGUGUUCUUGCUCUAUAA (SEQ ID NO: 32) | UUAUAGAGCAAGAACACUGUUUU (SEQ ID NO: 33) |
| AD- | TTR | asascag(Uhd)GfuUfCfUfugcucuausasa | VPusUfsauaGfagcaagaAfcAfcuguususu |
| 579804 | | (SEQ ID NO: 34) | (SEQ ID NO: 35) |
| AD- | TTR | asascagY180GfuUfCfUfugcucuausasa | VPusUfsauaGfagcaagaAfcAfcuguususu |
| 1953661 | | (SEQ ID NO: 36) | (SEQ ID NO: 37) |
| AD- | TTR | asascagY250GfuUfCfUfugcucuausasa | VPusUfsauaGfagcaagaAfcAfcuguususu |
| 1953662 | | (SEQ ID NO: 38) | (SEQ ID NO: 39) |
| AD- | m/rTTR | asascagY152GfuUfCfUfugcucuausasa | VPusUfsauaGfagcaagaAfcAfcuguususu |
| 1953667 | | (SEQ ID NO: 40) | (SEQ ID NO: 41) |
| aCSF | SOD1 | csasuuu(Uhd)AfaUfCfCfucacucuasasa | VPusUfsuagAfgUfGfaggaUfuAfaaaugsasg |
| | | (SEQ ID NO: 60) | (SEQ ID NO: 61) |

### Evaluation of Pharmacodynamics (PD) of Novel Lipid Ligand

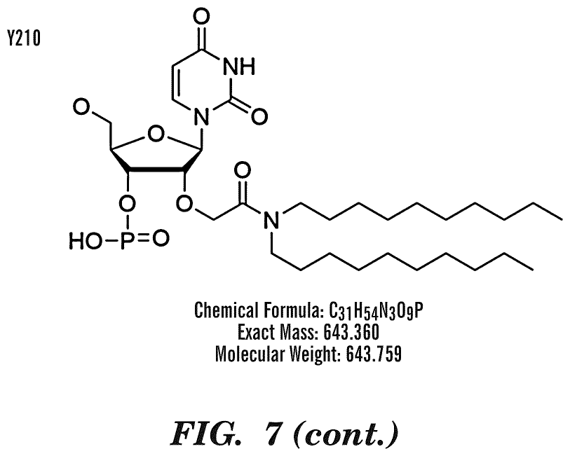

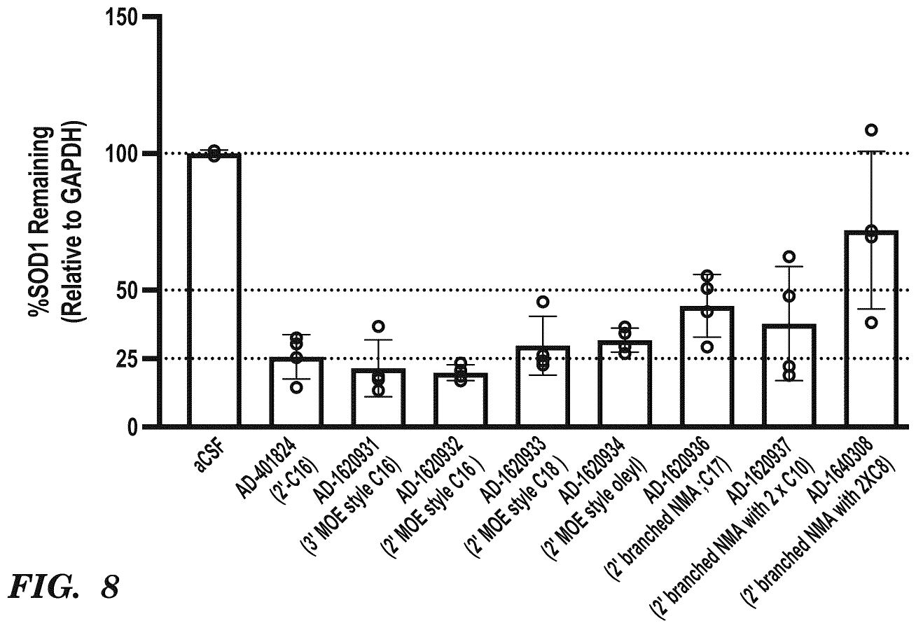

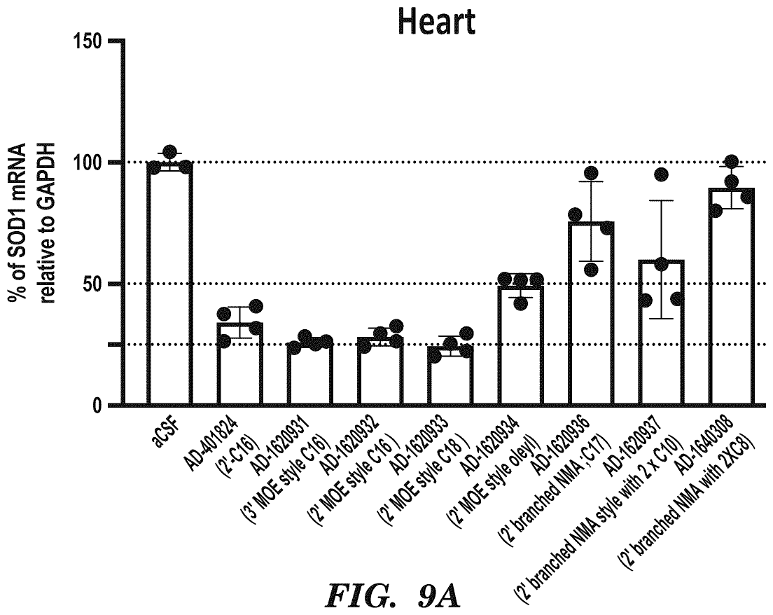

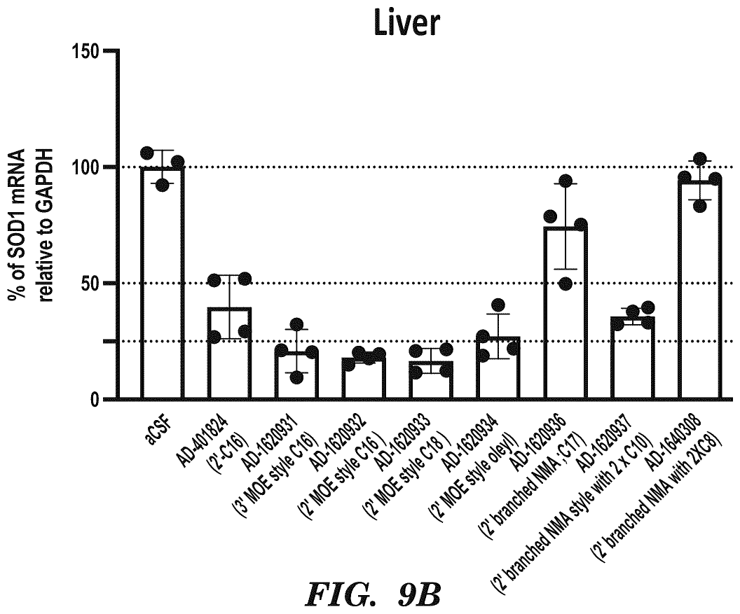

Pharmacodynamics of dsRNAs comprising exemplary monomers of the disclosure were evaluated. The study design is shown in Table 5. Results are shown in FIGS. 8, 9A and 9B.

TABLE 5
Experimental design for evaluating the pharmacodynamics novel lipids

| | | | # of | |
|---|---|---|---|---|
| Groups | Duplex | Dose | mice | Timepoint |
| 1 | aCSF | | 3 | Day 10 |
| 2 | AD-401824 (C16; current CNS template) | 150 ug | 4 | |
| 3 | AD-1620931.1 (3′ MOE style C16) | | 4 | |
| 4 | AD-1620932.1 (2′ MOE style C16) | | 4 | |
| 5 | AD-1620933.1 (2′ MOE style C18) | | 4 | |
| 6 | AD-1620934.1 (2′ MOE style oleyl) | | 4 | |
| 7 | AD-1620936.1 (2′ branched NMA style with total C17. | | 4 | |
| | Different branching pattern than the other two NMA styles) | | | |
| 8 | AD-1620937.1 (2′ branched NMA style with 2 × C10) | | 4 | |
| 9 | AD-1640308 (2′ branched NMA with 2 × C8: C16 total.) | | 4 | |

Results shows dsRNAs comprising exemplary monomers of the disclosure have comparable activities as the control (monomer C16) in brains (FIG. 8). Further, dsRNA molecules having strong RNAi activity in the brain also have robust RNAi activity in the heart (FIG. 9A) and liver (FIG. 9B).

### Central Nervous Activity in Rat IT

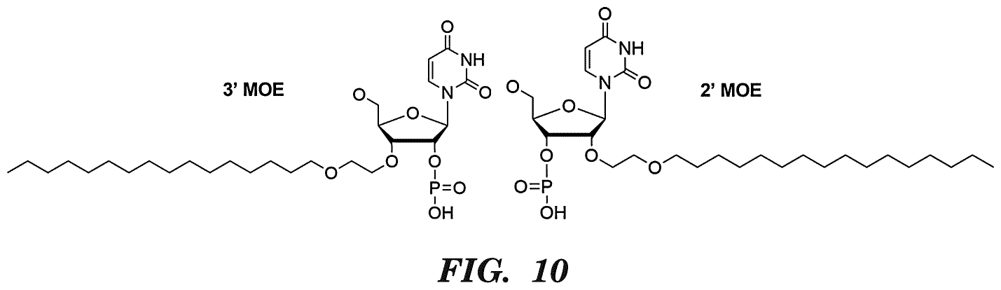

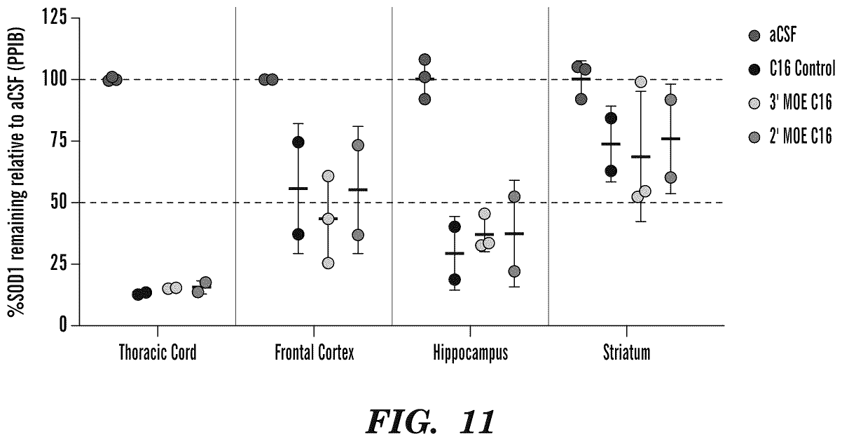

Central nervous system activity of dsRNA molecules comprising exemplary 3′-MOE and 2′-MOE style C16 monomers (FIG. 10) in rat IT was evaluated. The experimental deign is shown in Table 6. Results are shown in FIG. 11.

TABLE 6
Experimental design for evaluating CNS activity in rat IT

| Group | Duplex ID | Chemistry | Dose (mg) | Duration | Animals | Tissue Collection |
|---|---|---|---|---|---|---|
| 1 | aCSF | — | — | D 15 | N = 3 | *CNS (frozen for qPCR): |
| 9 | AD-401824 | C16 Control | 0.9 (in | | | striatum, prefrontal cortex, |
| 10 | AD-1620931 | 3′ MOE style C16 | 30 ul) | | | hippocampus, cerebellum, thoracic |
| 1 | AD-1620932 | 2′ MOE style C16 | | | | spine |
| | | | | | | *CNS (fixed for histology): |
| | | | | | | Left hemisphere |
| | | | | | | CSF and Plasma |
| | | | | | | *Periphery(frozen for qPCR): |
| | | | | | | heart, liver, kidney and lung |

Double-stranded RNA molecules comprising exemplary MOE style C16 monomers have similar activity as the control dsRNA. The 3′ and 2′ MOE style lipid conjugation have similar level of rSOD1 knockdown as the C16 control with dosing variation within groups (FIG. 11).

### Evaluation of Lipophilic Modification: SOD1

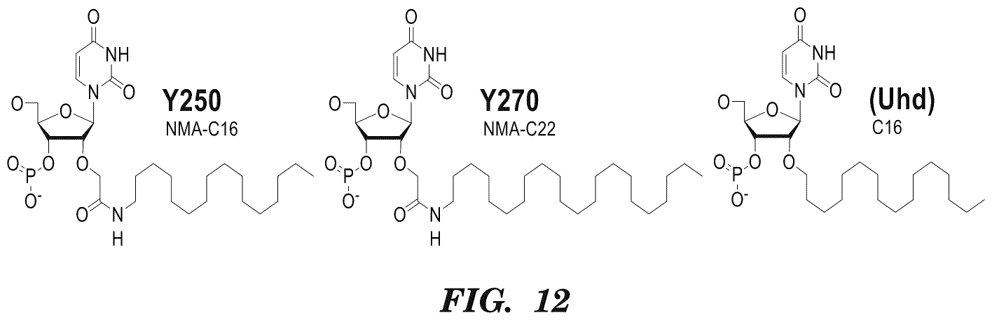

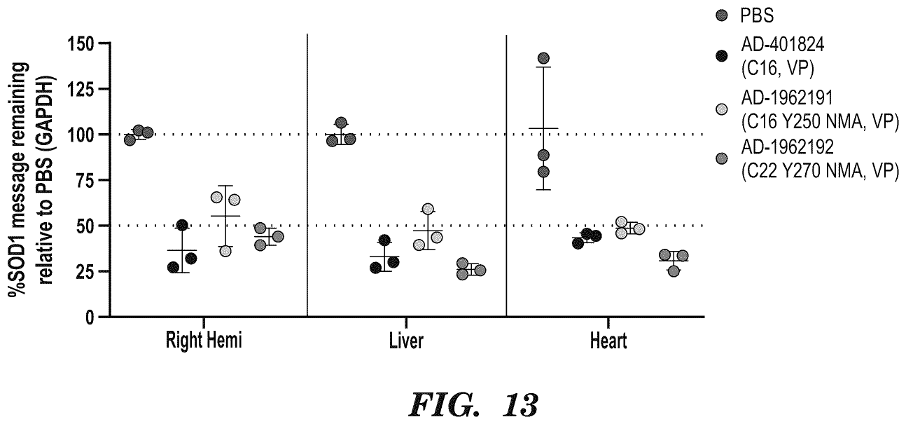

In this experiment, effect of lipophilic modification on SOD1 dsRNAs targeting SOD1 was evaluated. The experimental deign is shown in Table 7. Structures of monomers used are shown in FIG. 12 and the results are shown in FIG. 13.

TABLE 7
Experimental design for evaluation of lipophilic modifications on dsRNAs targeting SOD1

| | | | Dose | Route of | | | |
|---|---|---|---|---|---|---|---|
| Group | Duplex ID | Chemistry | (ug) | Admin* | Duration | Animals | Tissue Collection |
| 1 | PBS | — | — | — | D 15 | B57/Bl6 | CNS (frozen for qPCR): |
| 2 | AD-401824 | C16, VP | 100 | Freehand | | Female | Right hemisphere - olfactory |
| 3 | AD-1962191 | C16 Y250 NMA, VP | | ICV | | | bulbs, brainstem, cerebellum |
| 4 | AD-1962192 | C22 Y270 NMA, VP | | | | | removed |
| 5 | AD-1427063 | C16SO2-N(H) PN, VP | | | | | Periphery (frozen for qPCR): |
| | | | | | | | heart, liver |

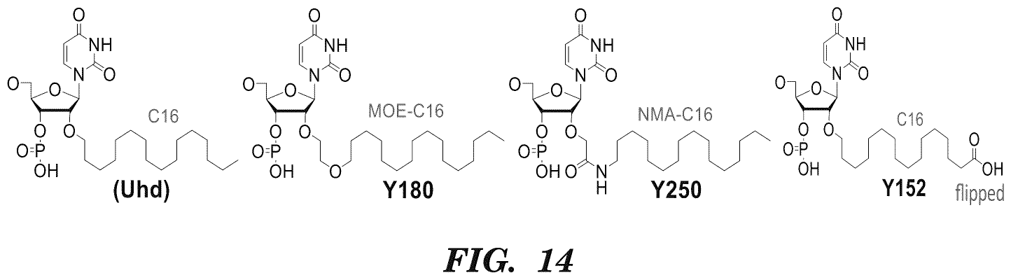

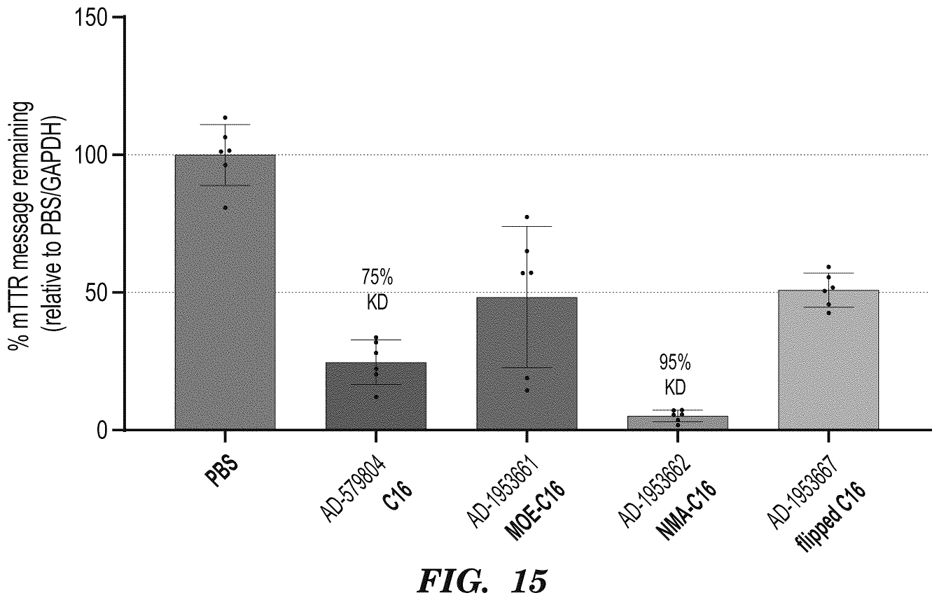

In this experiment, effect of chiral PS and C16 modifications in dsRNAs targeting mTTR in mouse eyes was evaluated. The experimental design is shown in Table 8. Structures of monomers used are shown in FIG. 14 and the results are shown in FIG. 15.

TABLE 8
Experimental design for evaluating effect on mTTR in mouse eye.

| | | | | | Dose | | |
|---|---|---|---|---|---|---|---|
| | | Dose (ug) | Sex/ | N (30 | Regimen/ | End of | Collections/ |
| Group | Test Article ID (TTR) | in 1 ul | Strain | mice) | Route | Study | Analysis |
| 1 | PBS | — | Female | 3 mice | Single | D 14 | Whole Eye (qPCR) |
| 2 | AD-579804.25 | 2.5 ug | C57BL/6 | (6 eyes) | D 0 | | 60 samples total |
| | C16, non-chiral 8PS | (2.5 mg/ml) | Mouse | | IVT | | |
| 3 | AD-1953661.1 | | | | | | |
| | Y180 (MOE-C16) | | | | | | |
| 4 | AD-1953662.1 | | | | | | |
| | Y250 (NMA-C16) | | | | | | |
| 5 | AD-1953667.1 | | | | | | |
| | Y152 (flipped C16) | | | | | | |

TABLE 9
Abbreviations used in sequences.

| | |
|---|---|
| Af | 2′-deoxy-2′-fluoroadenosine-3′-phosphate |
| Afs | 2′-deoxy-2′-fluoroadenosine-3′-phosphorothioate |
| Cf | 2′-deoxy-2′-fluorocytidine-3′-phosphate |
| Cfs | 2′-deoxy-2′-fluorocytidine-3′-phosphorothioate |
| Gf | 2′-deoxy-2′-fluoroguanosine-3′-phosphate |
| Gfs | 2′-deoxy-2′-fluoroguanosine-3′-phosphorothioate |
| Uf | 2′-deoxy-2′-fluorouridine-3′-phosphate |
| Ufs | 2′-deoxy-2′-fluorouridine-3′-phosphorothioate |
| a | 2′-O-methyladenosine-3′-phosphate |
| as | 2′-O-methyladenosine-3′-phosphorothioate |
| c | 2′-O-methylcytidine-3′-phosphate |
| cs | 2′-O-methylcytidine-3′-phosphorothioate |
| g | 2′-O-methylguanosine-3′-phosphate |
| gs | 2′-O-methylguanosine-3′-phosphorothioate |
| t | 2′-O-methyl-5-methyluridine-3′-phosphate |
| ts | 2′-O-methyl-5-methyluridine-3′-phosphorothioate |
| u | 2′-O-methyluridine-3′-phosphate |
| us | 2′-O-methyluridine-3′-phosphorothioate |
| dA | 2′-deoxyadenosine-3′-phosphate (Deoxy-Adenosine) |
| dAs | 2′-deoxyadenosine-3′-phosphorothioate |
| dC | 2′-deoxycytidine-3′-phosphate (Deoxy-Cytidine) |
| dCs | 2′-deoxycytidine-3′-phosphorothioate |
| dG | 2′-deoxyguanosine-3′-phosphate (Deoxy-Guanosine) |
| dG | 2′-deoxyguanosine-3′-phosphorothioate |
| dT | 2′-deoxyuridine-3′-phosphate (Deoxy-Thymidine) |
| dTs | 2′-deoxyuridine-3′-phosphorothioate |
| VP | 5′-E-vinylphosphonate |
| s | Phosphorothioate linkage - indicates modification of 3′-phosphate for 3′- |
| | phosphorothioate on the preceding nucleotide |
| ″•″ symbol | Phosphorothioate linkages - indicates modification of 3′-phosphate for 3′- |
| | phosphorothioate on the preceding nucleotide |
| L96 | (2S,4R)-1-[29-[2-(acetylamino)-2-deoxy-β-D-galactopyranosyl]oxy]-14,14- |
| | bis[[3-[[3-[[5-[[2-(acetylamino)-2-deoxy-β-D-galactopyranosyl]oxy]-1- |
| | oxopentyl]amino]propyl]amino]-3-oxopropoxy]methyl]-1,12,19,25-tetraoxo-16- |
| | oxa-13,20,24-triazanonacos-1-yl]-4-hydroxy-2-hydroxymethylpyrrolidine |
| | 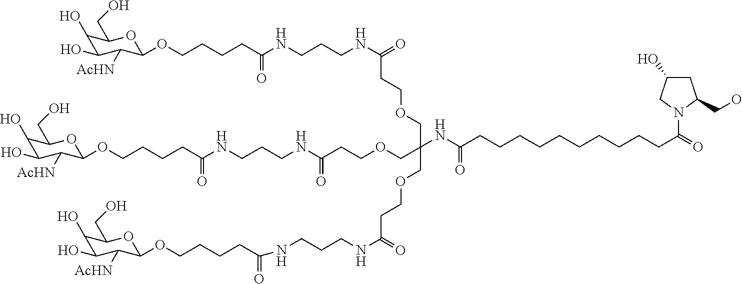 [SMILES: CC(=O)NC1C(OCCCCC(=O)NCCCNC(=O)CCOCC(COCCC(=O)NCCCNC(=O)CCCCOC2OC(CO)C(O)C(O)C2NC(C)=O)(COCCC(=O)NCCCNC(=O)CCCCOC2OC(CO)C(O)C(O)C2NC(C)=O)NC(=O)CCCCCCCCCCC(=O)N2C[C@H](O)C[C@H]2CO)OC(CO)C(O)C1O] |
| uL96 | 2′-O-methyluridine-3′-phosphate((2S,4R)-1-[29-[[2-(acetylamino)-2-deoxy-β-D- |
| | galactopyranosyl]oxy]-14,14-bis[[3-[[3-[5-[[2-(acetylamino)-2-deoxy-β-D- |
| | galactopyranosyl]oxy]-1-oxopentyl]amino]propyl]amino]-3-oxopropoxy]methyl]- |
| | 1,12,19,25-tetraoxo-16-oxa-13,20,24-triazanonacos-1-yl]-4-hydroxy-2- |
| | pyrrolidinyl)methyl ester |
| | 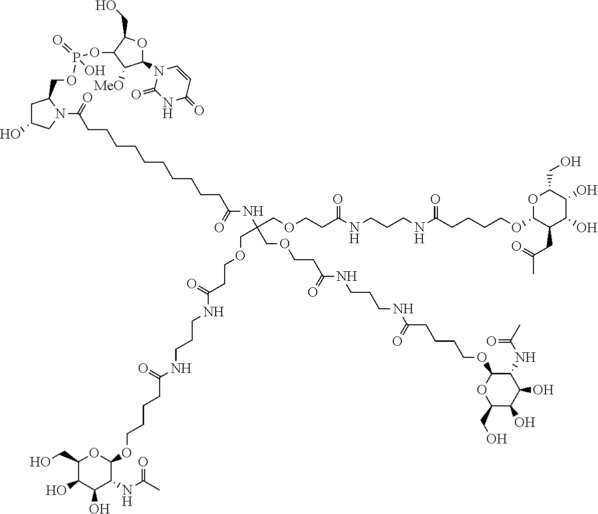 [SMILES: CO[C@@H]1C(OP(=O)(O)OC[C@@H]2C[C@@H](O)CN2C(=O)CCCCCCCCCCC(=O)NC(COCCC(=O)NCCCNC(=O)CCCCO[C@@H]2O[C@H](CO)[C@H](O)[C@H](O)[C@H]2CC(C)=O)(COCCC(=O)NCCCNC(=O)CCCCO[C@@H]2O[C@H](CO)[C@H](O)[C@H](O)[C@H]2NC(C)=O)COCCC(=O)NCCCNC(=O)CCCCO[C@@H]2O[C@H](CO)[C@H](O)[C@H](O)[C@H]2NC(C)=O)[C@@H](CO)O[C@H]1n1ccc(=O)[nH]c1=O] |
| Y179 | 3′-O-(2-(1-hexadecyloxy)ethyl)uridine-2′-phosphate |
| | 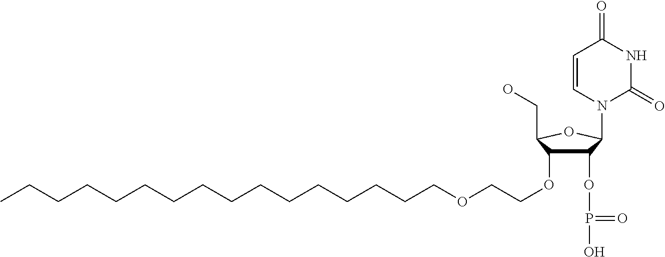 |
| Y180 | 2′-O-(2-(1-hexadecyloxy)ethyl)uridine-3′-phosphate |
| | 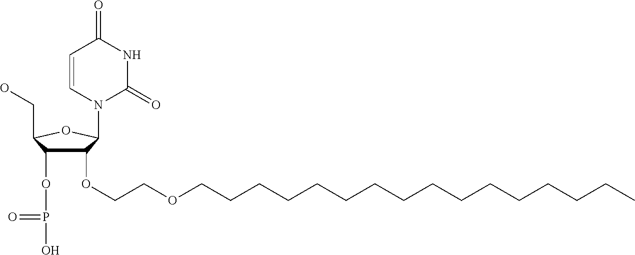 |
| Y182 | 2′-O-(2-(1-octadecyloxy)ethyl)uridine-3′-phosphate |
| | 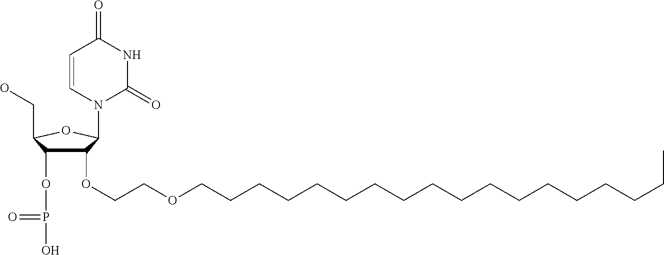 |
| Y184 | 2′-O-(2-(1-octadec-9Z-enoxy)ethyl)uridine-3′-phosphate |
| | 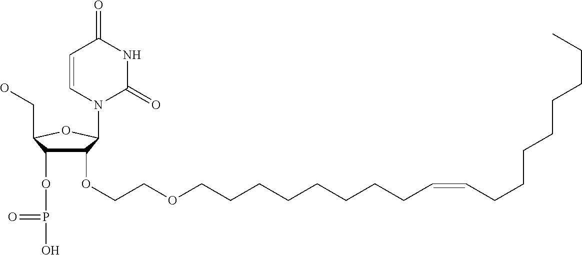 |
| Y208 | 2′-O-(2-oxo-2-(N-heptadec-9-ylamino)ethyl)uridine-3′-phosphate |
| | 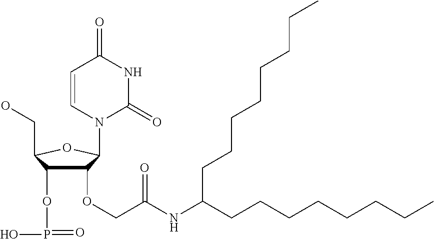 |
| Y209 | 2′-O-(2-oxo-2-(N,N-dioctan-1-ylamino)ethyl)-uridine-3′-phosphate |
| | 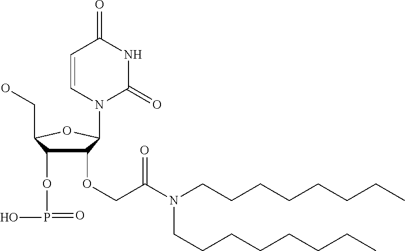 |
| Y210 | 2′-O-(2-oxo-2-(N,N-didecan-1-ylamino)ethyl)-uridine-3′-phosphate |
| | 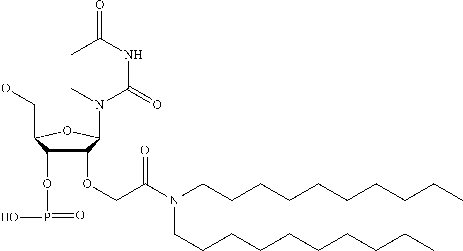 |
| Y250 | 2′-O-(2-oxo-2-(N-hexadec-1-ylamino)ethyl)uridine-3′-phosphate |
| C-16 amine | |
| conjugated U | 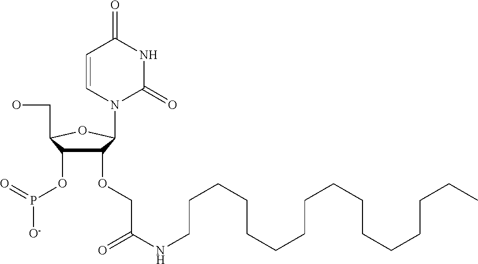 |
| Y270 | 2′-O-(2-oxo-2-(N-docosan-1-ylamino)ethyl)uridine-3′-phosphate |
| | 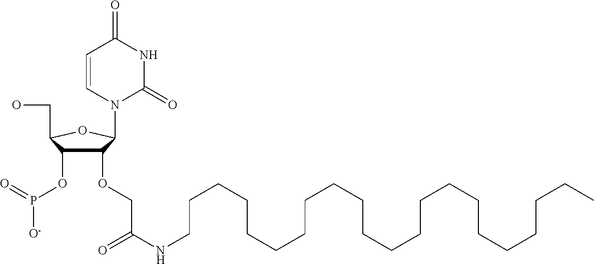 |
| Y152 | 2′-O-(15-carboxypentadec-1-yl)uridine-3′-phosphate |
| | 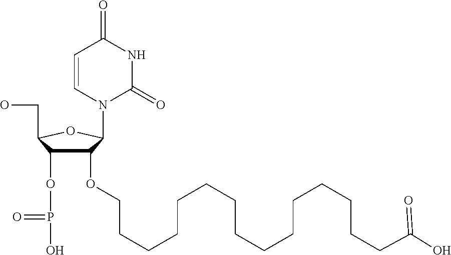 |
| (Ahd) | 2′-O-hexadecyladenosine-3′-phosphate |
| (Chd) | 2′-O-hexadecylcytidine-3′-phosphate |
| (Ghd) | 2′-O-hexadecylguanosine-3′-phosphate |
| (Uhd) | 2′-O-hexadecyluridine-3′-phosphate |
| (Ahds) | 2′-O-hexadecyladenosine-3′-phosphorothioate |
| (Chds) | 2′-O-hexadecylcytidine-3′-phosphorothioate |
| (Ghds) | 2′-O-hexadecylguanosine-3′-phosphorothioate |
| (Uhds) | 2′-O-hexadecyluridine-3′-phosphorothioate |
| Y800 | 2′-O-(2-oxo-2-(ethyloxy)ethyl)uridine-3′-phosphate |
| | 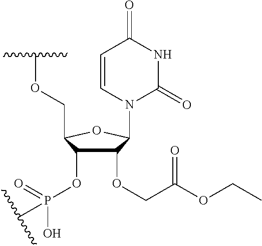 |
| Y801 | 2′-O-(2-oxo-2-(ethyloxy)ethyl)cytidine-3′-phosphate |
| | 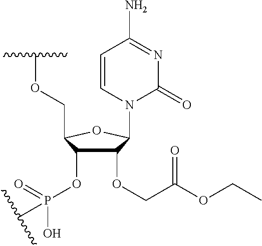 |
| Y802 | 2′-O-(2-oxo-2-(ethyloxy)ethyl)adenosine-3′-phosphate |
| | 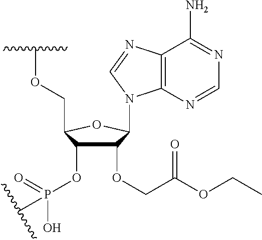 |
| Y803 | 2′-O-(2-oxo-2-(N-octadec-1-ylamino)ethyl)uridine-3′-phosphate |
| | 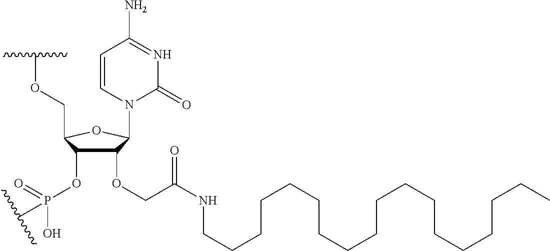 |
| Y804 | 2′-O-(2-oxo-2-(N-octadec-1-ylamino)ethyl)cytidine-3′-phosphate |
| | 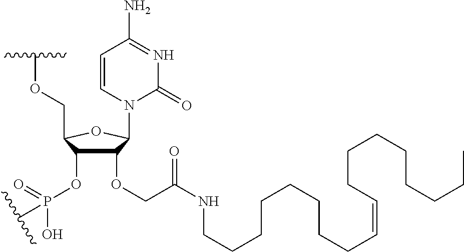 |
| Y805 | 2′-O-(2-oxo-2-(N-tetradec-1-ylamino)ethyl)cytidine-3′-phosphate |
| | 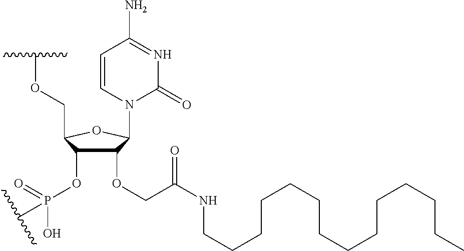 |

* 3′-terminal nucleotides are 3′-OH unless conjugated to (L) or otherwise indicate

All of the U.S. patents, U.S. patent application publications, foreign patents, foreign patent applications and non-patent publications referred to in this specification are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

What is claimed is:

1. A compound of Formula (I): 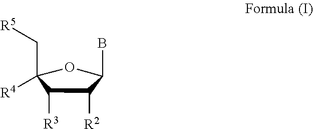
wherein:
B is an optionally modified nucleobase;
R² is R^{MA}, hydrogen, hydroxyl, protected hydroxyl, phosphate group, reactive phosphorous group, halogen, optionally substituted C₁₋₃₀ alkyl, optionally substituted C₂₋₃₀alkenyl, optionally substituted C₂₋₃₀alkynyl, optionally substituted C₁₋₃₀ alkoxy (e.g., methoxy), alkoxyalkyl (e.g., 2-methoxyethyl), alkoxyalkylamine, alkoxyoxycarboxylate, amino, alkylamino, dialkylamiino, 5-8 membered heterocyclyl, —O—C₄₋₃₀alkyl-ON(CH₂R⁸)(CH₂R⁹), —O—C₄₋₃₀alkyl-ON(CH₂R⁸)(CH₂R₉), a ligand, a linker covalently bonded to one or more ligands, a solid support, a linker or a linker covalently bonded to a solid support;
R^{MA} is —O(CH₂)ₘ₁—X^{M′}—R^{M′} or —O(CH₂)ₙ₁—C(Y^{M})N(R^{N′})(R^{N″})
Y^{M} is O or S;
X^{M′} is N(R^{MX}), O, or S, wherein R^{MX} is hydrogen or R^{M′};
R^{M′} is optionally substituted C₆₋₃₀ alkyl, optionally substituted C₆₋₃₀alkenyl, optionally substituted C₆₋₃₀alkynyl, optionally substituted 3-8 membered heterocyclylC₃₋₃₀alkyl, optionally substituted C₃₋₁₀cycloalkylC₃₋₃₀alkyl; optionally substituted arylC₃₋₃₀alkyl, optionally substituted heteroarylC₃₋₃₀alkyl, optionally substituted C₁₋₃₀ alkoxyC₁₋₃₀alkyl, —(CH₂CH₂O)_{mq}—R^{MQ}, a lipid, a ligand, a linker, or a linker to one or more ligands, wherein mq is an integer selected from 1-10 and R^{MQ} is hydrogen or C₁₋₆alkyl;
optionally, R^{M′} is terminally substituted with an anionic group or a cationic group;
m1 is an integer from 1 to 10;
n1 is an integer from 1 to 10;
R^{N′} and R^{N″} independently are hydrogen, optionally substituted C₆₋₃₀ alkyl, optionally substituted C₆₋₃₀alkenyl, optionally substituted C₆₋₃₀alkynyl, or optionally substituted C₃₋₃₀cycloalkyl; a lipid, a ligand, a linker, or a linker to one or more ligands, provided that at least one of R^{N′} and R^{N″} is not hydrogen;
R³ is R^{MA}, hydrogen, hydroxyl, protected hydroxyl, phosphate group, reactive phosphorous group, halogen, optionally substituted C₁₋₃₀ alkyl, optionally substituted C₂₋₃₀alkenyl, optionally substituted C₂₋₃₀alkynyl, optionally substituted C₁₋₃₀ alkoxy (e.g., methoxy), alkoxyalkyl (e.g., 2-methoxyethyl), alkoxyalkylamine, alkoxyoxycarboxylate, amino, alkylamino, dialkylamiino, 5-8 membered heterocyclyl, —O—C₄₋₃₀alkyl-ON(CH₂R⁸)(CH₂R⁹), —O—C₄₋₃₀alkyl-ON(CH₂R⁸)(CH₂R⁹), a ligand, a linker covalently bonded to one or more ligands, a solid support, a linker or a linker covalently bonded to a solid support;
R⁴ is R^{MA}, hydrogen, optionally substituted C₁₋₆alkyl, optionally substituted C₂₋₆alkenyl, optionally substituted C₂₋₆alkynyl, or optionally substituted C₁₋₆alkoxy;
R⁵ is R^{MA}, hydrogen, hydroxyl, protected hydroxyl, optionally substituted C₁₋₃₀ alkyl, optionally substituted C₂₋₃₀alkenyl, optionally substituted C₂₋₃₀alkynyl, optionally substituted C₁₋₃₀ alkoxy, optionally substituted 3-8 membered heterocyclyl (e.g., morpholin-1-yl, piperidin-1-yl, or pyrrolidin-1-yl), halogen, alkoxyalkyl (e.g., 2-methoxyethyl), alkoxyalkylamine, alkoxyoxycarboxylate, amino, alkylamino, dialkylamino, —O—C₄₋₃₀alkyl-ON(CH₂R⁸)(CH₂R⁹), —O—C₄₋₃₀alkyl-ON(CH₂R⁸)(CH₂R⁹), vinylphosphonate (VP) group (e.g., ═CH—X^{P}, X^{P} is a phosphate group), C₃₋₆ cycloalkylphosphonate (e.g., cyclopropylphosphonate), monophosphate ((HO)₂(O)P—O-5′), diphosphate ((HO)₂(O)P—O—P(HO)(O)—O-5′), triphosphate ((HO)₂(O)P—O—(HO)(O)P—O—P(HO)(O)—O-5′); monothiophosphate (phosphorothioate, (HO)₂(S)P—O-5′), monodithiophosphate (phosphorodithioate; (HO)(HS)(S)P—O-5′), phosphorothiolate ((HO)₂(O)P—S-5′); alpha-thiotriphosphate; beta-thiotriphosphate; gamma-thiotriphosphate; phosphoramidates ((HO)₂(O)P—NH-5′, (HO)(NH₂)(O)P—O-5′), alkylphosphonates [(R^{P})(OH)(O)P—O-5′, R^{P} is optionally substituted C₁₋₃₀ alkyl, e.g., methyl, ethyl, isopropyl, or propyl)], alkyletherphosphonates [(R_{P1})(OH)(O)P—O-5′, R^{P1} is alkoxyalkyl, e.g., methoxymethyl (CH₂OMe) or ethoxymethyl], (HO)₂(X)P—O[—(CH₂)ₐ—O—P(X)(OH)—O]_{b}-5′ or (HO)₂(X)P—O[—(CH₂)ₐ—P(X)(OH)—O]_{b}-5′ or (HO)₂(X)P—[—(CH₂)ₐ—O—P(X)(OH)—O]_{b}—5′, or optionally substituted alkyl, and dialkyl terminal phosphates and phosphate mimics (e.g., HO[—(CH₂)ₐ—O—P(X)(OH)—O]_{b}-5′, H₂N[—(CH₂)ₐ—O—P(X)(OH)—O]_{b}-5′ H[—(CH₂)ₐ—O—P(X)(OH)—O]_{b}-5′, Me₂N[—(CH₂)ₐ—O—P(X)(OH)—O]_{b}-5′, HO[—(CH₂)ₐ—P(X)(OH)—O]_{b}-5′, H₂N[—(CH₂)ₐ—P(X)(OH)—O]_{b}-5′, H[—(CH₂)ₐ—P(X)(OH)—O]_{b}-5′, Me₂N[—(CH₂)ₐ—P(X)(OH)—O]_{b}-5′, wherein
X is O or S;
a and b are each independently 1-10;
each R⁸ and R⁹ is independently H, a targeting ligand (e.g., GalNac), a pharmacokinetics modifier, optionally substituted C₁₋₃₀ alkyl, optionally substituted C₁₋₃₀ alkenyl, or optionally substituted C₁₋₃₀alkynyl; and
provided:
(i) at least one of R², R³, R⁴ and R⁵ is R^{MA};
(ii) only one of R², R³, R⁴ and R⁵ is R^{MA};
(iii) only one of R² and R³ is reactive phosphorous group, a solid support, or a linker covalently bonded to a solid support;
(iv) when R² is —OCH₂CH₂—O—R^{M′}; R⁵ is hydroxyl or protected hydroxyl; R⁴ is H; and R³ is hydroxyl, protected hydroxyl, a phosphate group or a reactive phosphorous group, then R^{M′} is not unsubstituted C₆₋₂₁ alkyl, unsubstituted C₆₋₂₁ alkenyl, or unsubstituted C₆₋₂₁alkynyl; and
(v) when R² is —O(CH₂)ₙ₁—C(O)N(R^{N′})(R^{N″}); n1 is 1; R⁵ is hydroxyl or protected hydroxyl; R⁴ is H; and R³ is hydroxyl, protected hydroxyl, a phosphate group or a reactive phosphorous group; and one of R^{N′} and R^{N″} is H, then the other of R^{N′} and R^{N″} is not —(CH₂)₆CH₃, —(CH₂)₇CH₃, —(CH₂)₈CH₃, —(CH₂)₅NHCOCF₃, —(CH₂)₆NHCOCF₃, —(CH₂)₇NHCOCF₃, —(CH₂)₅N(CH₃)₂, —(CH₂)₆N(CH₃)₂ or —(CH₂)₇N(CH₃)₂.

2. The compound of claim 1, wherein R² is —O(CH₂)ₘ₁—X^{M′}—R^{M′} (e.g., —OCH₂CH₂—X^{M′}—R^{M′}).

3. The compound of claim 2, wherein X^{M′} is O.

4. The compound of claim 2, wherein X^{M′} is S.

5. The compound of claim 1, wherein R² is —O(CH₂)ₙ₁—C(Y^{M})N(R^{N′})(R^{N″}).

6. The compound of claim 5, wherein n1 is 1.

7. The compound of claim 5, wherein n1 is 2.

8. The compound of any one of claims 2-7, wherein R³ is hydrogen, hydroxyl, protected hydroxyl, halogen, optionally substituted C₁₋₃₀ alkoxy, a reactive phosphorous group, a solid support, a linker, or a linker covalently attached to a solid support.

9. The compound of any one of claims 2-8, wherein R³ is hydrogen, hydroxyl, protected hydroxyl, a reactive phosphorous group, a solid support, a linker, or a linker covalently attached to a solid support.

10. The compound of any one of claims 2-9, wherein R³ is a reactive phosphorous group, a solid support, a linker, or a linker covalently attached to a solid support.

11. The compound of any one of 2-10, wherein R³ is a reactive phosphorous or a linker covalently attached to a solid support.

12. The compound of any one of claims 2-11, wherein R³ is a reactive phosphorous group (e.g., a phosphoramidite, such as [(2-cyanoethyl)-(N,N-diisopropyl)]-phosphoramidite or [(ß-thiobenzoylethyl)-(1-pyrrolidinyl)]-thiophosphoramidite).

13. The compound of any one of claims 2-11, wherein R³ is a linker covalently attached to a solid support.

14. The compound of any one of claims 2-13, wherein R⁵ is hydroxyl, protected hydroxyl, optionally substituted C₁₋₃₀ alkoxy, vinylphosphonate (VP) group, monophosphate, diphosphate, triphosphate, monothiophosphate (phosphorothioate), monodithiophosphate, phosphorothiolate, alpha-thiotriphosphate, beta-thiotriphosphate, gamma-thiotriphosphate, phosphoramidate, alkylphosphonate, alkyletherphosphonate, dialkyl terminal phosphate or phosphate mimic.

15. The compound of any one of claims 2-14, wherein R⁵ is hydroxyl, protected hydroxyl, vinylphosphonate (VP) group, cyclopropylphosphonate, monophosphate, diphosphate, triphosphate, monothiophosphate (phosphorothioate), monodithiophosphate, phosphorothiolate, alpha-thiotriphosphate, beta-thiotriphosphate, gamma-thiotriphosphate, phosphoramidates, alkylphosphonate, alkyletherphosphonate, dialkyl terminal phosphate, or a phosphate mimic.

16. The compound of any one of claims 2-15, wherein R⁵ is hydroxyl or protected hydroxyl.

17. The compound of claim 1, wherein R³ is —O(CH₂)ₘ₁—X^{M′}—R^{M′} (e.g., —OCH₂CH₂—X^{M′}—R^{M′}).

18. The compound of claim 17, wherein X^{M′} is O.

19. The compound of claim 17, wherein X^{M′} is S.

20. The compound of claim 1, wherein R³ is —O(CH₂)ₙ₁—C(Y^{M})N(R^{N′})(R^{N″}).

21. The compound of claim 20, wherein n1 is 1.

22. The compound of claim 20, wherein n1 is 2.

23. The compound of any one of claims 17-22, wherein R² is hydrogen, hydroxyl, protected hydroxyl, halogen, optionally substituted C₁₋₃₀ alkoxy, a reactive phosphorous group, a solid support, a linker, or a linker covalently attached to a solid support.

24. The compound of any one of claims 17-23, wherein R² is hydrogen, hydroxyl, protected hydroxyl, halogen, a reactive phosphorous group, a solid support, a linker, or a linker covalently attached to a solid support.

25. The compound of any one of claims 17-24, wherein R² is hydrogen, hydroxyl, protected hydroxyl, a reactive phosphorous group, a solid support, a linker, or a linker covalently attached to a solid support.

26. The compound of any one of claims 17-25, wherein R² is a reactive phosphorous group, a solid support, a linker, or a linker covalently attached to a solid support.

27. The compound of any one of 17-26, wherein R² is a reactive phosphorous or a linker covalently attached to a solid support.

28. The compound of any one of claims 17-27, wherein R² is a reactive phosphorous group (e.g., a phosphoramidite, such as [(2-cyanoethyl)-(N,N-diisopropyl)]-phosphoramidite or [(ß-thiobenzoylethyl)-(1-pyrrolidinyl)]-thiophosphoramidite.

29. The compound of any one of claims 17-27, wherein R² is a linker covalently attached to a solid support.

30. The compound of any one of claims 17-29, wherein R⁵ is hydroxyl, protected hydroxyl, optionally substituted C₁₋₃₀ alkoxy, vinylphosphonate (VP) group, monophosphate, diphosphate, triphosphate, monothiophosphate (phosphorothioate), monodithiophosphate, phosphorothiolate, alpha-thiotriphosphate, beta-thiotriphosphate, gamma-thiotriphosphate, phosphoramidate, alkylphosphonate, alkyletherphosphonate, dialkyl terminal phosphate or phosphate mimic.

31. The compound of any one of claims 17-30, wherein R⁵ is hydroxyl, protected hydroxyl, vinylphosphonate (VP) group, cyclopropylphosphonate, monophosphate, diphosphate, triphosphate, monothiophosphate (phosphorothioate), monodithiophosphate, phosphorothiolate, alpha-thiotriphosphate, beta-thiotriphosphate, gamma-thiotriphosphate, phosphoramidates, alkylphosphonate, alkyletherphosphonate, dialkyl terminal phosphate, or a phosphate mimic.

32. The compound of any one of claims 17-31, wherein R⁵ is hydroxyl or protected hydroxyl.

33. The compound of claim 1, wherein R⁵ is —O(CH₂)ₘ₁—X^{M′}—R^{M′} (e.g., —OCH₂CH₂—X^{M′}—R^{M′}).

34. The compound of claim 33, wherein X^{M′} is O.

35. The compound of claim 33, wherein X^{M′} is S.

36. The compound of claim 1, wherein R⁵ is —O(CH₂)ₙ₁—C(Y^{M})N(R^{N′})(R^{N″}).

37. The compound of claim 36, wherein n1 is 1.

38. The compound of claim 36, wherein n1 is 2.

39. The compound of any one of claims 33-38, wherein R² is hydrogen, hydroxyl, protected hydroxyl, halogen, optionally substituted C₁₋₃₀ alkoxy, a reactive phosphorous group, a solid support, a linker, or a linker covalently attached to a solid support.

40. The compound of any one of claims 33-39, wherein R² is hydrogen, hydroxyl, protected hydroxyl, halogen, a reactive phosphorous group, a solid support, a linker, or a linker covalently attached to a solid support.

41. The compound of any one of claims 33-40, wherein R² is hydrogen, hydroxyl, protected hydroxyl, a reactive phosphorous group, a solid support, a linker, or a linker covalently attached to a solid support.

42. The compound of any one of claims 33-41, wherein R² is a reactive phosphorous group, a solid support, a linker, or a linker covalently attached to a solid support.

43. The compound of any one of 33-42, wherein R² is a reactive phosphorous or a linker covalently attached to a solid support.

44. The compound of any one of claims 33-43, wherein R² is a reactive phosphorous group (e.g., a phosphoramidite, such as [(2-cyanoethyl)-(N,N-diisopropyl)]-phosphoramidite or [(ß-thiobenzoylethyl)-(1-pyrrolidinyl)]-thiophosphoramidite.

45. The compound of any one of claims 33-44, wherein R² is a linker covalently attached to a solid support.

46. The compound of any one of claims 33-45, wherein R³ is hydrogen, hydroxyl, protected hydroxyl, halogen, or optionally substituted C₁₋₃₀ alkoxy.

47. The compound of any one of claims 33-46, wherein R³ is hydroxyl or protected hydroxyl.

48. The compound of any one of claims 33-38, wherein R³ is hydrogen, hydroxyl, protected hydroxyl, halogen, optionally substituted C₁₋₃₀ alkoxy, a reactive phosphorous group, a solid support, a linker, or a linker covalently attached to a solid support.

49. The compound of claim 48, wherein R³ is hydrogen, hydroxyl, protected hydroxyl, halogen, a reactive phosphorous group, a solid support, a linker, or a linker covalently attached to a solid support.

50. The compound of any one of claims 48-49, wherein R³ is hydrogen, hydroxyl, protected hydroxyl, a reactive phosphorous group, a solid support, a linker, or a linker covalently attached to a solid support.

51. The compound of any one of claims 48-50, wherein R³ is a reactive phosphorous group, a solid support, a linker, or a linker covalently attached to a solid support.

52. The compound of any one of 48-51, wherein R³ is a reactive phosphorous or a linker covalently attached to a solid support.

53. The compound of any one of claims 48-52, wherein R³ is a reactive phosphorous group (e.g., a phosphoramidite, such as [(2-cyanoethyl)-(N,N-diisopropyl)]-phosphoramidite or [(ß-thiobenzoylethyl)-(1-pyrrolidinyl)]-thiophosphoramidite.

54. The compound of any one of claims 48-52, wherein R³ is a linker covalently attached to a solid support.

55. The compound of any one of claims 48-54, wherein R² is hydrogen, hydroxyl, protected hydroxyl, halogen, or optionally substituted C₁₋₃₀ alkoxy.

56. The compound of any one of claims 48-55, wherein R² is hydroxyl or protected hydroxyl.

57. The compound of any one of claims 1-56, wherein R⁴ is H.

58. A compound of Formula (I-A): 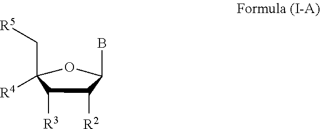
wherein:
B is an optionally modified nucleobase;
R² is —O(CH₂)ₘ₁—X^{M′}—R^{M′} (e.g., —OCH₂CH₂—X^{M′}—R^{M′}), hydroxyl, protected hydroxyl, reactive phosphorous group, a solid support, a linker or a linker covalently bonded to a solid support;
R³ is —O(CH₂)ₘ₁—X^{M′}—R^{M′} (e.g., —OCH₂CH₂—X^{M′}—R^{M′}), hydroxyl, protected hydroxyl, reactive phosphorous group, a solid support, a linker or a linker covalently bonded to a solid support;
R⁴ is —O(CH₂)ₘ₁—X^{M′}—R^{M′} (e.g., —OCH₂CH₂—X^{M′}—R^{M′}), hydrogen, optionally substituted C₁₋₆ alkyl, optionally substituted C₂₋₆alkenyl, optionally substituted C₂₋₆alkynyl, or optionally substituted C₁₋₆alkoxy; and
R⁵ is —O(CH₂)ₘ₁—X^{M′}—R^{M′} (e.g., —OCH₂CH₂—X^{M′}—R^{M′}), hydroxyl or protected hydroxyl;
R^{M′} is optionally substituted C₆₋₃₀ alkyl, optionally substituted C₆₋₃₀alkenyl, optionally substituted C₆₋₃₀alkynyl, optionally substituted 3-8 membered heterocyclylC₃₋₃₀alkyl, optionally substituted C₃₋₁₀cycloalkylC₃₋₃₀alkyl; optionally substituted arylC₃₋₃₀alkyl, optionally substituted heteroarylC₃₋₃₀alkyl, optionally substituted C₁₋₃₀ alkoxyC₁₋₃₀alkyl, —(CH₂CH₂O)_{mq}—R^{MQ}, a lipid, a ligand, a linker, or a linker to one or more ligands, wherein mq is an integer selected from 1-10 and R^{MQ} is hydrogen or C₁₋₆alkyl;
optionally, R^{M′} is terminally substituted with an anionic group or a cationic group;
X^{M′} is N(R^{MX}), O or S; and
provided:
(i) at least one of R², R³, R⁴ and R⁵ is —O(CH₂)ₘ₁—X^{M′}—R^{M′} (e.g., —OCH₂CH₂—X^{M′}—R^{M′});
(ii) only one of R², R³, R⁴ and R⁵ is —O(CH₂)ₘ₁—X^{M′}—R^{M′} (e.g., —OCH₂CH₂—X^{M′}—R^{M′});
(iii) only one of R² and R³ is reactive phosphorous group, a solid support, or a linker covalently bonded to a solid support; and
(iv) when R² is —OCH₂CH₂—O—R^{M′}; R⁵ is hydroxyl or protected hydroxyl; R⁴ is H; and R³ is hydroxyl, protected hydroxyl, a phosphate group or a reactive phosphorous group, then R^{M′} is not unsubstituted C₆₋₂₁ alkyl, unsubstituted C₆₋₂₁ alkenyl, or unsubstituted C₆₋₂₁alkynyl.

59. The compound of claim 58, wherein R² is —O(CH₂)ₘ₁—X^{M′}—R^{M′} (e.g., —OCH₂CH₂—X^{M′}—R^{M′}).

60. The compound of claim 59, wherein R³ is a hydroxyl group.

61. The compound of claim 59, wherein R³ is a protected hydroxyl.

62. The compound of claim 61, wherein the protected hydroxyl is —OR^{Pro}, wherein R^{Pro} is selected from the group consisting of acetyl, benzyl, benzoyl, 2,6-dichlorobenzyl, t-butyldimethylsilyl, t-butyldiphenylsilyl, trimethylsilyl, triisopropylsilyl, mesylate, tosylate, 4,4′-dimethoxytrityl (DMT), 9-phenylxanthine-9-yl (Pixyl) and 9-(p-methoxyphenyl)xanthine-9-yl (MOX).

63. The compound of claim 62, wherein the protected hydroxyl is —OR^{Pro}, wherein R^{Pro} is selected from the group consisting of acetyl, benzyl, t-butyldimethylsilyl, t-butyldiphenylsilyl, trimethylsilyl, triisopropylsilyl, and dimethoxytrityl.

64. The compound of claim 63, wherein the protected hydroxyl is —OR^{Pro}, wherein R^{Pro} is selected from the group consisting of t-butyldimethylsilyl, t-butyldiphenylsilyl, trimethylsilyl, and triisopropylsilyl.

65. The compound of claim 59 wherein R³ is a reactive phosphorous group.

66. The compound of claim 65, wherein the reactive phosphorous group is —OP(OR^{P})(N(R^{P2})₂), —OP(SR^{P})(N(R^{P2})₂), —OP(O)(OR^{P})(N(R^{P2})₂), —OP(S)(OR^{P})(N(R^{P2})₂), —OP(O)(SR^{P})(N(R^{P2})₂), —OP(O)(OR^{P})H, —OP(S)(OR^{P})H, —OP(O)(SR^{P})H, —OP(O)(OR^{P})R^{P3}, —OP(S)(OR^{P})R^{P3}, or —OP(O)(SR^{P})R^{P3}.

67. The compound of claim 66, wherein the reactive phosphorous group is —OP(OR^{P})(N(R^{P2})₂).

68. The compound of claim 67, wherein the reactive phosphorous group is OP(OR^{P})(N(R^{P2})₂), wherein R^{P} is cyanoethyl (—CH₂CH₂CN) and each R^{P2} is isopropyl or both R^{P2} taken together with the nitrogen atom to which they are attached form an optionally substituted 3-8 membered heterocyclyl.

69. The compound of claim 59, wherein R³ is a linker or a linker attached to a solid support.

70. The compound of any one of claims 59-69, wherein R⁵ is hydroxyl.

71. The compound of any one of claims 59-69, wherein R⁵ is a protected hydroxyl.

72. The compound of claim 71, wherein the protected hydroxyl is —OR^{Pro}, wherein R^{Pro} is an oxygen protecting group.

73. The compound of claim 72, wherein R^{Pro} is selected from the group consisting of acetyl, benzyl, benzoyl, 2,6-dichlorobenzyl, t-butyldimethylsilyl, t-butyldiphenylsilyl, trimethylsilyl, triisopropylsilyl, mesylate, tosylate, 4,4′-dimethoxytrityl (DMT), 9-phenylxanthine-9-yl (Pixyl) and 9-(p-methoxyphenyl)xanthine-9-yl (MOX).

74. The compound of claim 73, wherein R^{Pro} is 4,4′-dimethoxytrityl.

75. The compound of claim 58, wherein R³ is —O(CH₂)ₘ₁—X^{M′}—R^{M′} (e.g., —OCH₂CH₂—X^{M′}—R^{M′}).

76. The compound of claim 75, wherein R² is a hydroxyl group.

77. The compound of claim 75, wherein R² is a protected hydroxyl.

78. The compound of claim 77, wherein the protected hydroxyl is —OR^{Pro}, wherein R^{Pro} is selected from the group consisting of acetyl, benzyl, benzoyl, 2,6-dichlorobenzyl, t-butyldimethylsilyl, t-butyldiphenylsilyl, trimethylsilyl, triisopropylsilyl, mesylate, tosylate, 4,4′-dimethoxytrityl (DMT), 9-phenylxanthine-9-yl (Pixyl) and 9-(p-methoxyphenyl)xanthine-9-yl (MOX).

79. The compound of claim 78, wherein the protected hydroxyl is —OR^{Pro}, wherein R^{Pro} is selected from the group consisting of acetyl, benzyl, t-butyldimethylsilyl, t-butyldiphenylsilyl, trimethylsilyl, triisopropylsilyl, and dimethoxytrityl.

80. The compound of claim 79, wherein the protected hydroxyl is —OR^{Pro}, wherein R^{Pro} is selected from the group consisting of t-butyldimethylsilyl, t-butyldiphenylsilyl, trimethylsilyl, and triisopropylsilyl.

81. The compound of claim 75, wherein R² is a reactive phosphorous group.

82. The compound of claim 81, wherein the reactive phosphorous group is —OP(OR^{P})(N(R^{P2})₂), —OP(SR^{P})(N(R^{P2})₂), —OP(O)(OR^{P})(N(R^{P2})₂), —OP(S)(OR^{P})(N(R^{P2})₂), —OP(O)(SR^{P})(N(R^{P2})₂), —OP(O)(OR^{P})H, —OP(S)(OR^{P})H, —OP(O)(SR^{P})H, —OP(O)(OR^{P})R^{P3}, —OP(S)(OR^{P})R^{P3}, or —OP(O)(SR^{P})R^{P3}.

83. The compound of claim 82, wherein the reactive phosphorous group is —OP(OR^{P})(N(R^{P2})₂).

84. The compound of claim 83, wherein the reactive phosphorous group is OP(OR^{P})(N(R^{P2})₂), wherein R^{P} is cyanoethyl (—CH₂CH₂CN) and each R^{P2} is isopropyl or both R^{P2} taken together with the nitrogen atom to which they are attached form an optionally substituted 3-8 membered heterocyclyl.

85. The compound of claim 84, wherein R² is a linker or a linker attached to a solid support.

86. The compound of any one of claims 75-85, wherein R⁵ is hydroxyl.

87. The compound of any one of claims 75-85, wherein R⁵ is a protected hydroxyl.

88. The compound of claim 87, wherein the protected hydroxyl is —OR^{Pro}, wherein R^{Pro} is an oxygen protecting group.

89. The compound of claim 88, wherein R^{Pro} is selected from the group consisting of acetyl, benzyl, benzoyl, 2,6-dichlorobenzyl, t-butyldimethylsilyl, t-butyldiphenylsilyl, trimethylsilyl, triisopropylsilyl, mesylate, tosylate, 4,4′-dimethoxytrityl (DMT), 9-phenylxanthine-9-yl (Pixyl) and 9-(p-methoxyphenyl)xanthine-9-yl (MOX).

90. The compound of claim 89, wherein R^{Pro} is 4,4′-dimethoxytrityl.

91. The compound of claim 58, wherein R⁵ is —O(CH₂)ₘ₁—X^{M′}—R^{M′} (e.g., —OCH₂CH₂—X^{M′}—R^{M′}).

92. The compound of claim 91, wherein R³ is a hydroxyl group.

93. The compound of claim 91, wherein R³ is a protected hydroxyl.

94. The compound of claim 93, wherein the protected hydroxyl is —OR^{Pro}, wherein R^{Pro} is selected from the group consisting of acetyl, benzyl, benzoyl, 2,6-dichlorobenzyl, t-butyldimethylsilyl, t-butyldiphenylsilyl, trimethylsilyl, triisopropylsilyl, mesylate, tosylate, 4,4′-dimethoxytrityl (DMT), 9-phenylxanthine-9-yl (Pixyl) and 9-(p-methoxyphenyl)xanthine-9-yl (MOX).

95. The compound of claim 94, wherein the protected hydroxyl is —OR^{Pro}, wherein R^{Pro} is selected from the group consisting of acetyl, benzyl, t-butyldimethylsilyl, t-butyldiphenylsilyl, trimethylsilyl, triisopropylsilyl, and dimethoxytrityl.

96. The compound of claim 95, wherein the protected hydroxyl is —OR^{Pro}, wherein R^{Pro} is selected from the group consisting of t-butyldimethylsilyl, t-butyldiphenylsilyl, trimethylsilyl, and triisopropylsilyl.

97. The compound of claim 96, wherein R³ is a reactive phosphorous group.

98. The compound of claim 97, wherein the reactive phosphorous group is —OP(OR^{P})(N(R^{P2})₂), —OP(SR^{P})(N(R^{P2})₂), —OP(O)(OR^{P})(N(R^{P2})₂), —OP(S)(OR^{P})(N(R^{P2})₂), —OP(O)(SR^{P})(N(R^{P2})₂), —OP(O)(OR^{P})H, —OP(S)(OR^{P})H, —OP(O)(SR^{P})H, —OP(O)(OR^{P})R^{P3}, —OP(S)(OR^{P})R^{P3}, or —OP(O)(SR^{P})R^{P3}.

99. The compound of claim 98, wherein the reactive phosphorous group is —OP(OR^{P})(N(R^{P2})₂).

100. The compound of claim 99, wherein the reactive phosphorous group is OP(OR^{P})(N(R^{P2})₂), wherein R^{P} is cyanoethyl (—CH₂CH₂CN) and each R^{P2} is isopropyl or both R^{P2} taken together with the nitrogen atom to which they are attached form an optionally substituted 3-8 membered heterocyclyl.

101. The compound of claim 91, wherein R³ is a linker or a linker attached to a solid support.

102. The compound of any one of claims 91-101, wherein R² is hydroxyl or protected hydroxyl.

103. The compound of claim 102, wherein the protected hydroxyl is —OR^{Pro}, wherein R^{Pro} is selected from the group consisting of acetyl, benzyl, benzoyl, 2,6-dichlorobenzyl, t-butyldimethylsilyl, t-butyldiphenylsilyl, trimethylsilyl, triisopropylsilyl, mesylate, tosylate, 4,4′-dimethoxytrityl (DMT), 9-phenylxanthine-9-yl (Pixyl) and 9-(p-methoxyphenyl)xanthine-9-yl (MOX).

104. The compound of claim 103, wherein the protected hydroxyl is —OR^{Pro}, wherein R^{Pro} is selected from the group consisting of acetyl, benzyl, t-butyldimethylsilyl, t-butyldiphenylsilyl, trimethylsilyl, triisopropylsilyl, and dimethoxytrityl.

105. The compound of claim 104, wherein the protected hydroxyl is —OR^{Pro}, wherein R^{Pro} is selected from the group consisting of t-butyldimethylsilyl, t-butyldiphenylsilyl, trimethylsilyl, and triisopropylsilyl.

106. The compound of claim 91, wherein R² is a hydroxyl group.

107. The compound of claim 91, wherein R² is a protected hydroxyl.

108. The compound of claim 107, wherein the protected hydroxyl is —OR^{Pro}, wherein R^{Pro} is selected from the group consisting of acetyl, benzyl, benzoyl, 2,6-dichlorobenzyl, t-butyldimethylsilyl, t-butyldiphenylsilyl, trimethylsilyl, triisopropylsilyl, mesylate, tosylate, 4,4′-dimethoxytrityl (DMT), 9-phenylxanthine-9-yl (Pixyl) and 9-(p-methoxyphenyl)xanthine-9-yl (MOX).

109. The compound of claim 108, wherein the protected hydroxyl is —OR^{Pro}, wherein R^{Pro} is selected from the group consisting of acetyl, benzyl, t-butyldimethylsilyl, t-butyldiphenylsilyl, trimethylsilyl, triisopropylsilyl, and dimethoxytrityl.

110. The compound of claim 109, wherein the protected hydroxyl is —OR^{Pro}, wherein R^{Pro} is selected from the group consisting of t-butyldimethylsilyl, t-butyldiphenylsilyl, trimethylsilyl, and triisopropylsilyl.

111. The compound of claim 110, wherein R² is a reactive phosphorous group.

112. The compound of claim 111, wherein the reactive phosphorous group is —OP(OR^{P})(N(R^{P2})₂), —OP(SR^{P})(N(R^{P2})₂), —OP(O)(OR^{P})(N(R^{P2})₂), —OP(S)(OR^{P})(N(R^{P2})₂), —OP(O)(SR^{P})(N(R^{P2})₂), —OP(O)(OR^{P})H, —OP(S)(OR^{P})H, —OP(O)(SR^{P})H, —OP(O)(OR^{P})R^{P3}, —OP(S)(OR^{P})R^{P3}, or —OP(O)(SR^{P})R^{P3}.

113. The compound of claim 112, wherein the reactive phosphorous group is —OP(OR^{P})(N(R^{P2})₂).

114. The compound of claim 113, wherein the reactive phosphorous group is OP(OR^{P})(N(R^{P2})₂), wherein R^{P} is cyanoethyl (—CH₂CH₂CN) and each R^{P2} is isopropyl or both R^{P2} taken together with the nitrogen atom to which they are attached form an optionally substituted 3-8 membered heterocyclyl.

115. The compound of claim 114, wherein R² is a linker or a linker attached to a solid support.

116. The compound of any one of claims 106-115, wherein R³ is hydroxyl or protected hydroxyl.

117. The compound of claim 116, wherein the protected hydroxyl is —OR^{Pro}, wherein R^{Pro} is selected from the group consisting of acetyl, benzyl, benzoyl, 2,6-dichlorobenzyl, t-butyldimethylsilyl, t-butyldiphenylsilyl, trimethylsilyl, triisopropylsilyl, mesylate, tosylate, 4,4′-dimethoxytrityl (DMT), 9-phenylxanthine-9-yl (Pixyl) and 9-(p-methoxyphenyl)xanthine-9-yl (MOX).

118. The compound of claim 117, wherein the protected hydroxyl is —OR^{Pro}, wherein R^{Pro} is selected from the group consisting of acetyl, benzyl, t-butyldimethylsilyl, t-butyldiphenylsilyl, trimethylsilyl, triisopropylsilyl, and dimethoxytrityl.

119. The compound of claim 118, wherein the protected hydroxyl is —OR^{Pro}, wherein R^{Pro} is selected from the group consisting of t-butyldimethylsilyl, t-butyldiphenylsilyl, trimethylsilyl, and triisopropylsilyl.

120. The compound of any one of claims 58-119, wherein R⁴ is H.

121. The compound of any one of claims 58-120, wherein X^{M′} is O.

122. A compound of Formula (I-B): 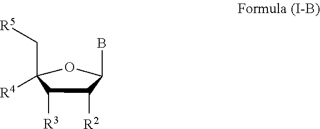
wherein:
B is an optionally modified nucleobase;
R² is —O(CH₂)ₙ₁—C(Y^{M})N(R^{N′})(R^{N″}), hydroxyl, protected hydroxyl, phosphate group, reactive phosphorous group, a solid support, a linker or a linker covalently bonded to a solid support;
R³ is —O(CH₂)ₙ₁—C(Y^{M})N(R^{N′})(R^{N″}), hydroxyl, protected hydroxyl, phosphate group, reactive phosphorous group, a solid support, a linker or a linker covalently bonded to a solid support;
R⁴ is —O(CH₂)ₙ₁—C(Y^{M})N(R^{N′})(R^{N″}), hydrogen, optionally substituted C₁₋₆alkyl, optionally substituted C₂₋₆alkenyl, optionally substituted C₂₋₆alkynyl, or optionally substituted C₁₋₆alkoxy;
R⁵ is —O(CH₂)ₙ₁—C(Y^{M})N(R^{N′})(R^{N″}), hydroxyl or protected hydroxyl;
n1 is an integer from 1 to 10;
Y^{M} is O or S; and
R^{N′} and R^{N″} independently are hydrogen, optionally substituted C₆₋₃₀ alkyl, optionally substituted C₆₋₃₀alkenyl, optionally substituted C₆₋₃₀alkynyl, or optionally substituted C₃₋₃₀cycloalkyl; a lipid, a ligand, a linker, or a linker to one or more ligands, provided that at least one of R^{N′} and R^{N″} is not hydrogen; and
provided:
(i) at least one of R², R³, R⁴ and R⁵ is —O(CH₂)ₙ₁—C(O)N(R^{N′})(R^{N″});
(ii) only one of R², R³, R⁴ and R⁵ is —O(CH₂)ₙ₁—C(O)N(R^{N′})(R^{N″});
(iii) only one of R² and R³ is reactive phosphorous group, a solid support, or a linker covalently bonded to a solid support; and
(iv) when R² is —O(CH₂)ₙ₁—C(O)N(R^{N′})(R^{N″}); n1 is 1; R³⁵ is hydroxyl or protected hydroxyl; R³⁴ is H; R³³ is hydroxyl, protected hydroxyl, phosphate group, or reactive phosphorous group; and one of R^{N′} and R^{N″} is H, then other of R^{N′} and R^{N″} is not —(CH₂)₆CH₃, —(CH₂)₇CH₃, —(CH₂)₈CH₃, —(CH₂)₅NHCOCF₃, —(CH₂)₆NHCOCF₃, —(CH₂)₇NHCOCF₃, —(CH₂)₅N(CH₃)₂, —(CH₂)₆N(CH₃)₂ or —(CH₂)₇N(CH₃)₂.

123. The compound of claim 122, wherein R² is —O(CH₂)ₙ₁—C(Y^{M})N(R^{N′})(R^{N″}), optionally Y^{M} is O.

124. The compound of claim 123, wherein R³ is a hydroxyl group.

125. The compound of claim 123, wherein R³ is a protected hydroxyl.

126. The compound of claim 125, wherein the protected hydroxyl is —OR^{Pro}, wherein R^{Pro} is selected from the group consisting of acetyl, benzyl, benzoyl, 2,6-dichlorobenzyl, t-butyldimethylsilyl, t-butyldiphenylsilyl, trimethylsilyl, triisopropylsilyl, mesylate, tosylate, 4,4′-dimethoxytrityl (DMT), 9-phenylxanthine-9-yl (Pixyl) and 9-(p-methoxyphenyl)xanthine-9-yl (MOX).

127. The compound of claim 126, wherein the protected hydroxyl is —OR^{Pro}, wherein R^{Pro} is selected from the group consisting of acetyl, benzyl, t-butyldimethylsilyl, t-butyldiphenylsilyl, trimethylsilyl, triisopropylsilyl, and dimethoxytrityl.

128. The compound of claim 127, wherein the protected hydroxyl is —OR^{Pro}, wherein R^{Pro} is selected from the group consisting of t-butyldimethylsilyl, t-butyldiphenylsilyl, trimethylsilyl, and triisopropylsilyl.

129. The compound of claim 128, wherein R³ is a reactive phosphorous group.

130. The compound of claim 129, wherein the reactive phosphorous group is —OP(OR^{P})(N(R^{P2})₂), —OP(SR^{P})(N(R^{P2})₂), —OP(O)(OR^{P})(N(R^{P2})₂), —OP(S)(OR^{P})(N(R^{P2})₂), —OP(O)(SR^{P})(N(R^{P2})₂), —OP(O)(OR^{P})H, —OP(S)(OR^{P})H, —OP(O)(SR^{P})H, —OP(O)(OR^{P})R^{P3}, —OP(S)(OR^{P})R^{P3}, or —OP(O)(SR^{P})R^{P3}.

131. The compound of claim 130, wherein the reactive phosphorous group is —OP(OR^{P})(N(R^{P2})₂).

132. The compound of claim 131, wherein the reactive phosphorous group is OP(OR^{P})(N(R^{P2})₂), wherein R^{P} is cyanoethyl (—CH₂CH₂CN) and each R^{P2} is isopropyl or both R^{P2} taken together with the nitrogen atom to which they are attached form an optionally substituted 3-8 membered heterocyclyl.

133. The compound of claim 123, wherein R³³ is a linker or a linker attached to a solid support.

134. The compound of any one of claims 123-133, wherein R⁵ is a protected hydroxyl.

135. The compound of claim 134, wherein the protected hydroxyl is —OR^{Pro}, wherein R^{Pro} is an oxygen protecting group.

136. The compound of claim 135, wherein R^{Pro} is selected from the group consisting of acetyl, benzyl, benzoyl, 2,6-dichlorobenzyl, t-butyldimethylsilyl, t-butyldiphenylsilyl, trimethylsilyl, triisopropylsilyl, mesylate, tosylate, 4,4′-dimethoxytrityl (DMT), 9-phenylxanthine-9-yl (Pixyl) and 9-(p-methoxyphenyl)xanthine-9-yl (MOX).

137. The compound of claim 136, wherein R^{Pro} is 4,4′-dimethoxytrityl.

138. The compound of claim 122, wherein R³ is —O(CH₂)ₙ₁—C(Y^{M})N(R^{N′})(R^{N″}), optionally Y^{M} is O.

139. The compound of claim 123, wherein R² is a hydroxyl group.

140. The compound of claim 123, wherein R² is a protected hydroxyl.

141. The compound of claim 140, wherein the protected hydroxyl is —OR^{Pro}, wherein R^{Pro} is selected from the group consisting of acetyl, benzyl, benzoyl, 2,6-dichlorobenzyl, t-butyldimethylsilyl, t-butyldiphenylsilyl, trimethylsilyl, triisopropylsilyl, mesylate, tosylate, 4,4′-dimethoxytrityl (DMT), 9-phenylxanthine-9-yl (Pixyl) and 9-(p-methoxyphenyl)xanthine-9-yl (MOX).

142. The compound of claim 141, wherein the protected hydroxyl is —OR^{Pro}, wherein R^{Pro} is selected from the group consisting of acetyl, benzyl, t-butyldimethylsilyl, t-butyldiphenylsilyl, trimethylsilyl, triisopropylsilyl, and dimethoxytrityl.

143. The compound of claim 142, wherein the protected hydroxyl is —OR^{Pro}, wherein R^{Pro} is selected from the group consisting of t-butyldimethylsilyl, t-butyldiphenylsilyl, trimethylsilyl, and triisopropylsilyl.

144. The compound of claim 143, wherein R² is a reactive phosphorous group.

145. The compound of claim 144, wherein the reactive phosphorous group is —OP(OR^{P})(N(R^{P2})₂), —OP(SR^{P})(N(R^{P2})₂), —OP(O)(OR^{P})(N(R^{P2})₂), —OP(S)(OR^{P})(N(R^{P2})₂), —OP(O)(SR^{P})(N(R^{P2})₂), —OP(O)(OR^{P})H, —OP(S)(OR^{P})H, —OP(O)(SR^{P})H, —OP(O)(OR^{P})R^{P3}, —OP(S)(OR^{P})R^{P3}, or —OP(O)(SR^{P})R^{P3}.

146. The compound of claim 145, wherein the reactive phosphorous group is —OP(OR^{P})(N(R^{P2})₂).

147. The compound of claim 146, wherein the reactive phosphorous group is OP(OR^{P})(N(R^{P2})₂), wherein R^{P} is cyanoethyl (—CH₂CH₂CN) and each R^{P2} is isopropyl or both R^{P2} taken together with the nitrogen atom to which they are attached form an optionally substituted 3-8 membered heterocyclyl.

148. The compound of claim 147, wherein R² is a linker or a linker attached to a solid support.

149. The compound of any one of claims 138-148, wherein R⁵ is hydroxyl.

150. The compound of any one of claims 138-148, wherein R⁵ is a protected hydroxyl.

151. The compound of any one of claim 150, wherein the protected hydroxyl is —OR^{Pro}, wherein R^{Pro} is an oxygen protecting group.

152. The compound of claim 151, wherein R^{Pro} is selected from the group consisting of acetyl, benzyl, benzoyl, 2,6-dichlorobenzyl, t-butyldimethylsilyl, t-butyldiphenylsilyl, trimethylsilyl, triisopropylsilyl, mesylate, tosylate, 4,4′-dimethoxytrityl (DMT), 9-phenylxanthine-9-yl (Pixyl) and 9-(p-methoxyphenyl)xanthine-9-yl (MOX).

153. The compound of claim 152, wherein R^{Pro} is 4,4′-dimethoxytrityl.

154. The compound of claim 122, wherein R⁵ is —O(CH₂)ₙ₁—C(Y^{M})N(R^{N′})(R^{N″}), optionally Y^{M} is O.

155. The compound of claim 154, wherein R³ is a hydroxyl group.

156. The compound of claim 154, wherein R³ is a protected hydroxyl.

157. The compound of claim 156, wherein the protected hydroxyl is —OR^{Pro}, wherein R^{Pro} is selected from the group consisting of acetyl, benzyl, benzoyl, 2,6-dichlorobenzyl, t-butyldimethylsilyl, t-butyldiphenylsilyl, trimethylsilyl, triisopropylsilyl, mesylate, tosylate, 4,4′-dimethoxytrityl (DMT), 9-phenylxanthine-9-yl (Pixyl) and 9-(p-methoxyphenyl)xanthine-9-yl (MOX).

158. The compound of claim 157, wherein the protected hydroxyl is —OR^{Pro}, wherein R^{Pro} is selected from the group consisting of acetyl, benzyl, t-butyldimethylsilyl, t-butyldiphenylsilyl, trimethylsilyl, triisopropylsilyl, and dimethoxytrityl.

159. The compound of claim 158, wherein the protected hydroxyl is —OR^{Pro}, wherein R^{Pro} is selected from the group consisting of t-butyldimethylsilyl, t-butyldiphenylsilyl, trimethylsilyl, and triisopropylsilyl.

160. The compound of claim 159, wherein R³ is a reactive phosphorous group.

161. The compound of claim 160, wherein the reactive phosphorous group is —OP(OR^{P})(N(R^{P2})₂), —OP(SR^{P})(N(R^{P2})₂), —OP(O)(OR^{P})(N(R^{P2})₂), —OP(S)(OR^{P})(N(R^{P2})₂), —OP(O)(SR^{P})(N(R^{P2})₂), —OP(O)(OR^{P})H, —OP(S)(OR^{P})H, —OP(O)(SR^{P})H, —OP(O)(OR^{P})R^{P3}, —OP(S)(OR^{P})R^{P3}, or —OP(O)(SR^{P})R^{P3}.

162. The compound of claim 161, wherein the reactive phosphorous group is —OP(OR^{P})(N(R^{P2})₂).

163. The compound of claim 162, wherein the reactive phosphorous group is OP(OR^{P})(N(R^{P2})₂), wherein R^{P} is cyanoethyl (—CH₂CH₂CN) and each R^{P2} is isopropyl or both R^{P2} taken together with the nitrogen atom to which they are attached form an optionally substituted 3-8 membered heterocyclyl.

164. The compound of claim 163, wherein R³ is a linker or a linker attached to a solid support.

165. The compound of any one of claims 154-164, wherein R² is hydroxyl or protected hydroxyl.

166. The compound of claim 165, wherein the protected hydroxyl is —OR^{Pro}, wherein R^{Pro} is selected from the group consisting of acetyl, benzyl, benzoyl, 2,6-dichlorobenzyl, t-butyldimethylsilyl, t-butyldiphenylsilyl, trimethylsilyl, triisopropylsilyl, mesylate, tosylate, 4,4′-dimethoxytrityl (DMT), 9-phenylxanthine-9-yl (Pixyl) and 9-(p-methoxyphenyl)xanthine-9-yl (MOX).

167. The compound of claim 166, wherein the protected hydroxyl is —OR^{Pro}, wherein R^{Pro} is selected from the group consisting of acetyl, benzyl, t-butyldimethylsilyl, t-butyldiphenylsilyl, trimethylsilyl, triisopropylsilyl, and dimethoxytrityl.

168. The compound of claim 167, wherein the protected hydroxyl is —OR^{Pro}, wherein R^{Pro} is selected from the group consisting of t-butyldimethylsilyl, t-butyldiphenylsilyl, trimethylsilyl, and triisopropylsilyl.

169. The compound of claim 154, wherein R² is a hydroxyl group.

170. The compound of claim 154, wherein R² is a protected hydroxyl.

171. The compound of claim 170, wherein the protected hydroxyl is —OR^{Pro}, wherein R^{Pro} is selected from the group consisting of acetyl, benzyl, benzoyl, 2,6-dichlorobenzyl, t-butyldimethylsilyl, t-butyldiphenylsilyl, trimethylsilyl, triisopropylsilyl, mesylate, tosylate, 4,4′-dimethoxytrityl (DMT), 9-phenylxanthine-9-yl (Pixyl) and 9-(p-methoxyphenyl)xanthine-9-yl (MOX).

172. The compound of claim 171, wherein the protected hydroxyl is —OR^{Pro}, wherein R^{Pro} is selected from the group consisting of acetyl, benzyl, t-butyldimethylsilyl, t-butyldiphenylsilyl, trimethylsilyl, triisopropylsilyl, and dimethoxytrityl.

173. The compound of claim 172, wherein the protected hydroxyl is —OR^{Pro}, wherein R^{Pro} is selected from the group consisting of t-butyldimethylsilyl, t-butyldiphenylsilyl, trimethylsilyl, and triisopropylsilyl.

174. The compound of claim 173, wherein R² is a reactive phosphorous group.

175. The compound of claim 174, wherein the reactive phosphorous group is —OP(OR^{P})(N(R^{P2})₂), —OP(SR^{P})(N(R^{P2})₂), —OP(O)(OR^{P})(N(R^{P2})₂), —OP(S)(OR^{P})(N(R^{P2})₂), —OP(O)(SR^{P})(N(R^{P2})₂), —OP(O)(OR^{P})H, —OP(S)(OR^{P})H, —OP(O)(SR^{P})H, —OP(O)(OR^{P})R^{P3}, —OP(S)(OR^{P})R^{P3}, or —OP(O)(SR^{P})R^{P3}.

176. The compound of claim 175, wherein the reactive phosphorous group is —OP(OR^{P})(N(R^{P2})₂).

177. The compound of claim 176, wherein the reactive phosphorous group is OP(OR^{P})(N(R^{P2})₂), wherein R^{P} is cyanoethyl (—CH₂CH₂CN) and each R^{P2} is isopropyl or both R^{P2} taken together with the nitrogen atom to which they are attached form an optionally substituted 3-8 membered heterocyclyl.

178. The compound of claim 177, wherein R² is a linker or a linker attached to a solid support.

179. The compound of any one of claims 169-178, wherein R³ is hydroxyl or protected hydroxyl.

180. The compound of claim 179, wherein the protected hydroxyl is —OR^{Pro}, wherein R^{Pro} is selected from the group consisting of acetyl, benzyl, benzoyl, 2,6-dichlorobenzyl, t-butyldimethylsilyl, t-butyldiphenylsilyl, trimethylsilyl, triisopropylsilyl, mesylate, tosylate, 4,4′-dimethoxytrityl (DMT), 9-phenylxanthine-9-yl (Pixyl) and 9-(p-methoxyphenyl)xanthine-9-yl (MOX).

181. The compound of claim 180, wherein the protected hydroxyl is —OR^{Pro}, wherein R^{Pro} is selected from the group consisting of acetyl, benzyl, t-butyldimethylsilyl, t-butyldiphenylsilyl, trimethylsilyl, triisopropylsilyl, and dimethoxytrityl.

182. The compound of claim 181, wherein the protected hydroxyl is —OR^{Pro}, wherein R^{Pro} is selected from the group consisting of t-butyldimethylsilyl, t-butyldiphenylsilyl, trimethylsilyl, and triisopropylsilyl.

183. The compound of any one of claims 122-182, wherein R³⁴ is H.

184. An oligonucleotide prepared using a compound of any one of claims 1-183 or 278-341.

185. An oligonucleotide comprising at least one nucleoside of Formula (II), 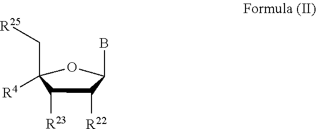
where:
B is an optionally modified nucleobase;
R²² is R^{MA}, a bond to an internucleotide linkage to a subsequent nucleotide, hydrogen, hydroxyl, protected hydroxyl, halogen, optionally substituted C₁₋₃₀ alkyl, optionally substituted C₂₋₃₀alkenyl, optionally substituted C₂₋₃₀alkynyl, optionally substituted C₁₋₃₀ alkoxy (e.g., methoxy), alkoxyalkyl (e.g., 2-methoxyethyl), alkoxyalkylamine, alkoxyoxycarboxylate, amino, alkylamino, dialkylamino, 5-8 membered heterocyclyl, —O—C₄₋₃₀alkyl-ON(CH₂R⁸)(CH₂R⁹), —O—C₄₋₃₀alkyl-ON(CH₂R⁸)(CH₂R⁹), a ligand, a linker covalently bonded to one or more ligands, a solid support, a linker or a linker covalently bonded to a solid support;
R^{MA} is —O(CH₂)ₘ₁—X^{M′}—R^{M′} or —O(CH₂)ₙ₁—C(Y^{M})N(R^{N′})(R^{N″})
Y^{M} is O or S;
X^{M′} is N(R^{MX}), O, or S, wherein R^{MX} is hydrogen or R^{M′};
R^{M′} is optionally substituted C₆₋₃₀ alkyl, optionally substituted C₆₋₃₀alkenyl, optionally substituted C₆₋₃₀alkynyl, optionally substituted 3-8 membered heterocyclylC₃₋₃₀alkyl, optionally substituted C₃₋₁₀cycloalkylC₃₋₃₀alkyl;
optionally substituted arylC₃₋₃₀alkyl, optionally substituted heteroarylC₃₋₃₀alkyl, optionally substituted C₁₋₃₀ alkoxyC₁₋₃₀alkyl, —(CH₂CH₂O)_{mq}—R^{MQ}, a lipid, a ligand, a linker, or a linker to one or more ligands, wherein mq is an integer selected from 1-10 and R^{MQ} is hydrogen or C₁₋₆alkyl;
optionally, R^{M′} is terminally substituted with an anionic group or a cationic group;
m1 is an integer from 1 to 10;
n1 is an integer from 1 to 10;
R^{N′} and R^{N″} independently are hydrogen, optionally substituted C₆₋₃₀ alkyl, optionally substituted C₆₋₃₀alkenyl, optionally substituted C₆₋₃₀alkynyl, or optionally substituted C₃₋₃₀cycloalkyl; a lipid, a ligand, a linker, or a linker to one or more ligands, provided that at least one of R^{N′} and R^{N″} is not hydrogen;
R³³ is R^{MA}, a bond to an internucleotide linkage to a subsequent nucleotide, hydrogen, hydroxyl, protected hydroxyl, halogen, optionally substituted C₁₋₃₀ alkyl, optionally substituted C₂₋₃₀alkenyl, optionally substituted C₂₋₃₀alkynyl, optionally substituted C₁₋₃₀ alkoxy (e.g., methoxy), alkoxyalkyl (e.g., 2-methoxyethyl), alkoxyalkylamine, alkoxyoxycarboxylate, amino, alkylamino, dialkylamino, 5-8 membered heterocyclyl, —O—C₄₋₃₀alkyl-ON(CH₂R⁸)(CH₂R⁹), —O—C₄₋₃₀alkyl-ON(CH₂R⁸)(CH₂R⁹), a ligand, a linker covalently bonded to one or more ligands, a solid support, a linker or a linker covalently bonded to a solid support;
R⁴ is R^{MA}, hydrogen, optionally substituted C₁₋₆alkyl, optionally substituted C₂₋₆alkenyl, optionally substituted C₂₋₆alkynyl, or optionally substituted C₁₋₆alkoxy;
R²⁵ is R^{MA}, a bond to an internucleotide linkage to a preceding nucleotide, hydrogen, hydroxyl, protected hydroxyl, optionally substituted C₁₋₃₀ alkyl, optionally substituted C₂₋₃₀alkenyl, optionally substituted C₂₋₃₀alkynyl, optionally substituted C₁₋₃₀ alkoxy, optionally substituted 3-8 membered heterocyclyl (e.g., morpholin-1-yl, piperidin-1-yl, or pyrrolidin-1-yl), halogen, alkoxyalkyl (e.g., 2-methoxyethyl), alkoxyalkylamine, alkoxyoxycarboxylate, amino, alkylamino, dialkylamino, —O—C₄₋₃₀alkyl-ON(CH₂R⁸)(CH₂R⁹), —O—C₄₋₃₀alkyl-ON(CH₂R⁸)(CH₂R⁹), vinylphosphonate (VP) group (e.g., ═CH—X^{P}, X^{P} is a phosphate group), C₃₋₆ cycloalkylphosphonate (e.g., cyclopropylphosphonate), monophosphate ((HO)₂(O)P—O-5′), diphosphate ((HO)₂(O)P—O—P(HO)(O)—O-5′), triphosphate ((HO)₂(O)P—O—(HO)(O)P—O—P(HO)(O)—O-5′); monothiophosphate (phosphorothioate, (HO)₂(S)P—O-5′), monodithiophosphate (phosphorodithioate; (HO)(HS)(S)P—O-5′), phosphorothiolate ((HO)₂(O)P—S-5′); alpha-thiotriphosphate; beta-thiotriphosphate; gamma-thiotriphosphate; phosphoramidates ((HO)₂(O)P—NH-5′, (HO)(NH₂)(O)P—O-5′), alkylphosphonates [(R^{P})(OH)(O)P—O-5′, R^{P} is optionally substituted C₁₋₃₀ alkyl, e.g., methyl, ethyl, isopropyl, or propyl)], alkyletherphosphonates [(R′)(OH)(O)P—O-5′, R^{P1} is alkoxyalkyl, e.g., methoxymethyl (CH₂OMe) or ethoxymethyl], (HO)₂(X)P—O[—(CH₂)ₐ—O—P(X)(OH)—O]_{b}-5′ or (HO)₂(X)P—O[—(CH₂)ₐ—P(X)(OH)—O]_{b}-5′ or (HO)₂(X)P—[—(CH₂)ₐ—O—P(X)(OH)—O]_{b}—5′, or optionally substituted alkyl, and dialkyl terminal phosphates and phosphate mimics (e.g., HO[—(CH₂)ₐ—O—P(X)(OH)—O]_{b}—5′, H₂N[—(CH₂)ₐ—O—P(X)(OH)—O]_{b}-5′, H[—(CH₂)ₐ—O—P(X)(OH)—O]_{b}-5′, Me₂N[—(CH₂)ₐ—O—P(X)(OH)—O]_{b}-5′, HO[—(CH₂)ₐ—P(X)(OH)—O]_{b}-5′, H₂N[—(CH₂)ₐ—P(X)(OH)—O]_{b}-5′, H[—(CH₂)ₐ—P(X)(OH)—O]_{b}-5′, Me₂N[—(CH₂)ₐ—P(X)(OH)—O]_{b}-5′, wherein
X is O or S;
a and b are each independently 1-10;
each R⁸ and R⁹ is independently H, a targeting ligand (e.g., GalNac), a pharmacokinetics modifier, optionally substituted C₁₋₃₀ alkyl, optionally substituted C₁₋₃₀ alkenyl, or optionally substituted C₁₋₃₀alkynyl; and
provided:
(i) at least one of R²², R²³, R²⁴ and R²⁵ is R^{MA};
(ii) only one of R²², R²³, R²⁴ and R²⁵ is R^{MA};
(iii) only one of R²² and R²³ is a solid support, a linker covalently bonded to a solid support or a bond to an internucleotide linkage to a subsequent nucleotide;
(iv) when both of R²² and R²³ are not bond to an internucleotide linkage to a subsequent nucleotide, then R²⁵ is a bond to an internucleotide linkage to a preceding nucleotide;
(v) when R²² is —OCH₂CH₂—X^{M′}—R^{M′}; R²⁵ is hydroxyl, protected hydroxyl or a bond to a bond to an internucleotide linkage to a preceding nucleotide; R⁴ is H; R²³ is hydroxyl, protected hydroxyl, a bond to an internucleotide linkage to a subsequent nucleotide, a solid support, a linker, or a linker covalently attached to a solid support, and at least one of R²³ and R²⁵ is a bond to an internucleotide linkage, then R^{M′} is not an unsubstituted C₅₋₂₁ alkyl, unsubstituted C₅₋₂₁alkenyl, or unsubstituted C₅₋₂₁alkynyl; and
(vi) when R²² is —O(CH₂)ₙ₁—C(O)N(R^{N′})(R^{N″}); n1 is 1; R⁵ hydroxyl, protected hydroxyl or a bond to a bond to an internucleotide linkage to a preceding nucleotide; R⁴ is H; R³ is hydroxyl, protected hydroxyl, a bond to an internucleotide linkage to a subsequent nucleotide, a solid support, a linker, or a linker covalently attached to a solid support, and at least one of R²³ and R²⁵ is a bond to an internucleotide linkage; one of R^{N′} and R^{N″} is H, and at least one of R²³ and R²⁵ is a bond to an internucleotide linkage, and then the other of R^{N′} and R^{N″} is not —(CH₂)₆CH₃, —(CH₂)₇CH₃, —(CH₂)₈CH₃, —(CH₂)₅NHCOCF₃, —(CH₂)₆NHCOCF₃, —(CH₂)₇NHCOCF₃, —(CH₂)₅N(CH₃)₂, —(CH₂)₆N(CH₃)₂ or —(CH₂)₇N(CH₃)₂.

186. The oligonucleotide of claim 185, wherein R²² is —O(CH₂)ₘ₁—X^{M′}—R^{M′} (e.g., —OCH₂CH₂—X^{M′}—R^{M′}).

187. The oligonucleotide of claim 186, wherein X^{M′} is O.

188. The oligonucleotide of claim 186, wherein X^{M′} is S.

189. The oligonucleotide of claim 185, wherein R²² is —O(CH₂)ₙ₁—C(Y^{M})N(R^{N′})(R^{N″}).

190. The oligonucleotide of claim 189, wherein n1 is 1.

191. The oligonucleotide of claim 189, wherein n1 is 2.

192. The oligonucleotide of any one of claims 186-191, wherein R²³ is a bond to an internucleotide linkage to a subsequent nucleotide, hydrogen, hydroxyl, protected hydroxyl, halogen, optionally substituted C₁₋₃₀ alkoxy, a solid support, a linker, or a linker covalently attached to a solid support.

193. The oligonucleotide of any one of claims 186-192, wherein R²³ is a bond to an internucleotide linkage to a subsequent nucleotide, hydroxyl, protected hydroxyl, a solid support, a linker, or a linker covalently attached to a solid support.

194. The oligonucleotide of any one of claims 186-193, wherein R²³ is a bond to an internucleotide linkage to a subsequent nucleotide.

195. The oligonucleotide of any one of claims 186-194, wherein R²³ is a linker covalently attached to a solid support.

196. The oligonucleotide of claim 185, wherein R²³ is —O(CH₂)ₘ₁—X^{M′}—R^{M′} (e.g., —OCH₂CH₂—X^{M′}—R^{M′}).

197. The oligonucleotide of claim 196, wherein X^{M′} is O.

198. The oligonucleotide of claim 196, wherein X^{M′} is S.

199. The oligonucleotide of claim 185, wherein R²³ is —O(CH₂)ₘ₁—C(Y^{M})N(R^{N′})(R^{N″}).

200. The oligonucleotide of claim 199, wherein n1 is 1.

201. The oligonucleotide of claim 199, wherein n1 is 2.

202. The oligonucleotide of any one of claims 196-201, wherein R²² is a bond to an internucleotide linkage to a subsequent nucleotide, hydrogen, hydroxyl, protected hydroxyl, halogen, optionally substituted C₁₋₃₀ alkoxy, a solid support, a linker, or a linker covalently attached to a solid support.

203. The oligonucleotide of any one of claims 196-202, wherein R²² is a bond to an internucleotide linkage to a subsequent nucleotide, hydroxyl, protected hydroxyl, a solid support, a linker, or a linker covalently attached to a solid support.

204. The oligonucleotide of any one of claims 196-203, wherein R²² is a bond to an internucleotide linkage to a subsequent nucleotide.

205. The oligonucleotide of any one of claims 196-203, wherein R²² is a linker covalently attached to a solid support.

206. The oligonulceotide of any one of claims 185-205, wherein R²⁴ is H.

207. The oligonucleotide of claim 185, wherein R²⁴ is —O(CH₂)ₘ₁—X^{M′}—R^{M′} (e.g., —OCH₂CH₂—X^{M′}—R^{M′}).

208. The oligonucleotide of claim 207, wherein X^{M′} is O.

209. The oligonucleotide of claim 207, wherein X^{M′} is S.

210. The oligonucleotide of claim 185, wherein R²⁴ is —O(CH₂)ₙ₁—C(Y^{M})N(R^{N′})(R^{N″}).

211. The oligonucleotide of claim 210, wherein n1 is 1.

212. The oligonucleotide of claim 211, wherein n1 is 2.

213. The oligonucleotide of any one of claims 207-212, wherein R²³ is a bond to an internucleotide linkage to a subsequent nucleotide, hydrogen, hydroxyl, protected hydroxyl, halogen, optionally substituted C₁₋₃₀ alkoxy, a solid support, a linker, or a linker covalently attached to a solid support.

214. The oligonucleotide of any one of claims 207-213, wherein R²³ is a bond to an internucleotide linkage to a subsequent nucleotide, hydroxyl, protected hydroxyl, a solid support, a linker, or a linker covalently attached to a solid support.

215. The oligonucleotide of any one of claims 213-214, wherein R²² is hydrogen, hydroxyl, protected hydroxyl, halogen, or optionally substituted C₁₋₃₀ alkyl.

216. The oligonucleotide of any one of claims 213-215, wherein R²² is hydrogen, hydroxyl or protected hydroxyl.

217. The oligonucleotide of any one of claims 207-212, wherein R²² is a bond to an internucleotide linkage to a subsequent nucleotide, hydrogen, hydroxyl, protected hydroxyl, halogen, optionally substituted C₁₋₃₀ alkoxy, a solid support, a linker, or a linker covalently attached to a solid support.

218. The oligonucleotide of acclaim 217, wherein R²² is a bond to an internucleotide linkage to a subsequent nucleotide, hydroxyl, protected hydroxyl, a solid support, a linker, or a linker covalently attached to a solid support.

219. The oligonucleotide of any one of claims 217-218, wherein R²³ is hydrogen, hydroxyl, protected hydroxyl, halogen, or optionally substituted C₁₋₃₀ alkyl.

220. The oligonucleotide of any one of claims 217-219, wherein R²³ is hydrogen, hydroxyl or protected hydroxyl.

221. The oligonucleotide of any one of claims 185-220, wherein R²⁵ is a bond to an internucleotide linkage to a preceding nucleotide, hydroxyl, protected hydroxyl, optionally substituted C₁₋₃₀ alkoxy, vinylphosphonate (VP) group, monophosphate, diphosphate, triphosphate, monothiophosphate (phosphorothioate), monodithiophosphate, phosphorothiolate, alpha-thiotriphosphate, beta-thiotriphosphate, gamma-thiotriphosphate, phosphoramidate, alkylphosphonate, alkyletherphosphonate, dialkyl terminal phosphate or phosphate mimic.

222. The oligonucleotide of any one of claims 185-221, wherein R²⁵ is a bond to an internucleotide linkage to a preceding nucleotide, hydroxyl, protected hydroxyl, vinylphosphonate (VP) group, cyclopropylphosphonate, monophosphate, diphosphate, triphosphate, monothiophosphate (phosphorothioate), monodithiophosphate, phosphorothiolate, alpha-thiotriphosphate, beta-thiotriphosphate, gamma-thiotriphosphate, phosphoramidates, alkylphosphonate, alkyletherphosphonate, dialkyl terminal phosphate, or a phosphate mimic.

223. The oligonucleotide of any one of claims 185-222, wherein R²⁵ is hydroxyl or protected hydroxyl.

224. The oligonucleotide of any one of claims 185-222, wherein R²⁵ is a bond to an internucleotide linkage to a preceding nucleotide.

225. The oligonucleotide of any one of claims 185-222, wherein R²⁵ is a vinylphosphonate (VP) group, cyclopropylphosphonate, monophosphate, diphosphate, triphosphate, monothiophosphate (phosphorothioate), monodithiophosphate, phosphorothiolate, alpha-thiotriphosphate, beta-thiotriphosphate, gamma-thiotriphosphate, phosphoramidates, alkylphosphonate, alkyletherphosphonate, dialkyl terminal phosphate, or a phosphate mimic.

226. The oligonucleotide of claim 185, wherein R²⁵ is —O(CH₂)ₘ₁—X^{M′}—R^{M′} (e.g., —OCH₂CH₂—X^{M′}—R^{M′}).

227. The oligonucleotide of claim 226, wherein X^{M′} is O.

228. The oligonucleotide of claim 226, wherein X^{M′} is S.

229. The oligonucleotide of claim 185, wherein R⁵ is —O(CH₂)ₙ₁—C(Y^{M})N(R^{N′})(R^{N″}).

230. The oligonucleotide of claim 229, wherein n1 is 1.

231. The oligonucleotide of claim 229, wherein n1 is 2.

232. The oligonucleotide of any one of claims 226-231, wherein R³ is a bond to an internucleotide linkage to a subsequent nucleotide, hydrogen, hydroxyl, protected hydroxyl, halogen, optionally substituted C₁₋₃₀ alkoxy, a solid support, a linker, or a linker covalently attached to a solid support.

233. The oligonucleotide of any one of claims 226-232, wherein R³ is a bond to an internucleotide linkage to a subsequent nucleotide, hydroxyl, protected hydroxyl, a solid support, a linker, or a linker covalently attached to a solid support.

234. The oligonucleotide of any one of claims 226-233, wherein R²² is hydrogen, hydroxyl, protected hydroxyl, halogen, or optionally substituted C₁₋₃₀ alkyl.

235. The oligonucleotide of any one of claims 226-234, wherein R²² is hydrogen, hydroxyl or protected hydroxyl.

236. The oligonucleotide of any one of claims 226-231, wherein R²² is a bond to an internucleotide linkage to a subsequent nucleotide, hydrogen, hydroxyl, protected hydroxyl, halogen, optionally substituted C₁₋₃₀ alkoxy, a solid support, a linker, or a linker covalently attached to a solid support.

237. The oligonucleotide of claim 236, wherein R²² is a bond to an internucleotide linkage to a subsequent nucleotide, hydroxyl, protected hydroxyl, a solid support, a linker, or a linker covalently attached to a solid support.

238. The oligonucleotide of claim 237, wherein R³ is hydrogen, hydroxyl, protected hydroxyl, halogen, or optionally substituted C₁₋₃₀ alkyl.

239. The oligonucleotide of claim 238, wherein R³ is hydrogen, hydroxyl or protected hydroxyl.

240. The oligonucleotide of any one of claims 185-206 or 221-239, wherein R²⁴ is H.

241. The oligonucleotide of any one of claims 185-240, wherein the oligonucleotide is attached to a solid support.

242. The oligonucleotide of any one of claims 185-241, wherein the oligonucleotide comprises from 3 to 50 nucleotides.

243. The oligonucleotide of any one of claims 185-242, wherein the oligonucleotide comprises at least one ribonucleotide.

244. The oligonucleotide of any one of claims 185-243, wherein the oligonucleotide comprises at least one 2′-deoxyribonucleotide.

245. The oligonucleotide of any one of claims 185-244, wherein the oligonucleotide comprises at least one nucleotide with a modified or non-natural nucleobase.

246. The oligonucleotide of any one of claims 185-245, wherein the oligonucleotide comprises at least one nucleotide with a modified ribose sugar in addition to the nucleotide of Formula (II).

247. The oligonucleotide of any one of claims 185-246, wherein the oligonucleotide comprises at least one nucleotide with a 2′-F ribose in addition to the nucleotide of Formula (II).

248. The oligonucleotide of any one of claims 185-247, wherein the oligonucleotide comprises at least one nucleotide with a 2′-OMe ribose in addition to the nucleotide of Formula (II).

249. The oligonucleotide of any one of claims 185-248, wherein the oligonucleotide comprises at least one nucleotide comprising a moiety other than a ribose sugar in addition to the nucleotide of Formula (II).

250. The oligonucleotide of any one of claims 185-249, wherein the oligonucleotide comprises at least one modified internucleotide linkage.

251. The oligonucleotide of any one of claims 185-250, wherein oligonucleotide comprises at least one ligand.

252. The oligonucleotide of any one of claims 185-251, wherein the oligonucleotide comprises at least one hydroxyl, phosphate or amino protecting group.

253. The oligonucleotide of any one of claims 185-252, wherein m1 is 2.

254. A double-stranded nucleic acid comprising a first oligonucleotide strand and a second oligonucleotide strand substantially complementary to the first strand, wherein the first or second strand is an oligonucleotide of any one of claims 185-253.

255. The double-stranded nucleic acid of claim 254, wherein the first and second strand are independently 15 to 25 nucleotides in length.

256. The double-stranded nucleic acid any one of claims 264-255, wherein double-stranded nucleic acid is capable of inducing RNA interference.

257. The double-stranded nucleic acid of any one of claims 264-256, wherein one or both strands have a 1-5 nucleotide overhang on its respective 5′-end or 3′-end.

258. The double-stranded nucleic acid of any one of claims 264-257, wherein only one strand has a 2 nucleotide overhang on its 5′-end or 3′-end.

259. The double-stranded nucleic acid of any one of claims 264-258, wherein only one strand has a 2 nucleotide overhand on its 3′-end.

260. A method of reducing the expression of a target gene in a subject, comprising administering to the subject either:
(i) a double-stranded RNA according to any one of claims 254-259, wherein the first strand or the second strand is complementary to a target gene; or
(ii) an oligonucleotide according to any one of claims 185-253, wherein the oligonucleotide is complementary to a target gene.

261. A method of preparing an oligonucleotide comprising at least nucleotide of Formula (II): 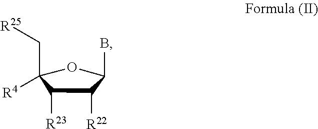
the method comprising reacting an oligonucleotide comprising nucleotide of Formula (II′): 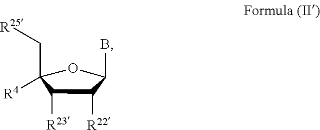
with an amine of formula HN(R^{N′})(R^{N″}),
where:
B is an optionally modified nucleobase;
R²² is —O(CH₂)ₙ₁—C(Y^{M})N(R^{N′})(R^{N″}), a bond to an internucleotide linkage to a subsequent nucleotide, hydrogen, hydroxyl, protected hydroxyl, halogen, optionally substituted C₁₋₃₀ alkyl, optionally substituted C₂₋₃₀alkenyl, optionally substituted C₂₋₃₀alkynyl, optionally substituted C₁₋₃₀ alkoxy (e.g., methoxy), alkoxyalkyl (e.g., 2-methoxyethyl), alkoxyalkylamine, alkoxyoxycarboxylate, amino, alkylamino, dialkylamino, 5-8 membered heterocyclyl, —O—C₄₋₃₀alkyl-ON(CH₂R⁸)(CH₂R⁹), —O—C₄₋₃₀alkyl-ON(CH₂R⁸)(CH₂R⁹), a ligand, a linker covalently bonded to one or more ligands, a solid support, a linker or a linker covalently bonded to a solid support;
Y^{M} is O or S;
R^{N′} and R^{N″} independently are hydrogen, optionally substituted C₆₋₃₀ alkyl, optionally substituted C₆₋₃₀alkenyl, optionally substituted C₆₋₃₀alkynyl, or optionally substituted C₃₋₃₀cycloalkyl, a lipid, a ligand, a linker, or a linker to one or more ligands, provided that at least one of R^{N′} and R^{N″} is not hydrogen;
n1 is an integer from 1 to 10;
R²³ is —O(CH₂)ₙ₁—C(O)N(R^{N′})(R^{N″}), a bond to an internucleotide linkage to a subsequent nucleotide, hydrogen, hydroxyl, protected hydroxyl, halogen, optionally substituted C₁₋₃₀ alkyl, optionally substituted C₂₋₃₀alkenyl, optionally substituted C₂₋₃₀alkynyl, optionally substituted C₁₋₃₀ alkoxy (e.g., methoxy), alkoxyalkyl (e.g., 2-methoxyethyl), alkoxyalkylamine, alkoxyoxycarboxylate, amino, alkylamino, dialkylamino, 5-8 membered heterocyclyl, —O—C₄₋₃₀alkyl-ON(CH₂R⁸)(CH₂R⁹), —O—C₄₋₃₀alkyl-ON(CH₂R⁸)(CH₂R⁹), a ligand, a linker covalently bonded to one or more ligands, a solid support, a linker or a linker covalently bonded to a solid support;
R⁴ is hydrogen, optionally substituted C₁₋₆alkyl, optionally substituted C₂₋₆alkenyl, optionally substituted C₂₋₆alkynyl, or optionally substituted C₁₋₆alkoxy;
R²⁵ is —O(CH₂)ₙ₁—C(O)N(R^{N′})(R^{N″}), a bond to an internucleotide linkage to a preceding nucleotide, hydrogen, hydroxyl, protected hydroxyl, optionally substituted C₁₋₃₀ alkyl, optionally substituted C₂₋₃₀alkenyl, optionally substituted C₂₋₃₀alkynyl, optionally substituted C₁₋₃₀ alkoxy, optionally substituted 3-8 membered heterocyclyl (e.g., morpholin-1-yl, piperidin-1-yl, or pyrrolidin-1-yl), halogen, alkoxyalkyl (e.g., 2-methoxyethyl), alkoxyalkylamine, alkoxyoxycarboxylate, amino, alkylamino, dialkylamino, —O—C₄₋₃₀alkyl-ON(CH₂R⁸)(CH₂R⁹), —O—C₄₋₃₀alkyl-ON(CH₂R⁸)(CH₂R⁹), vinylphosphonate (VP) group (e.g., ═CH—X^{P}, X^{P} is a phosphate group), C₃₋₆ cycloalkylphosphonate (e.g., cyclopropylphosphonate), monophosphate ((HO)₂(O)P—O-5′), diphosphate ((HO)₂(O)P—O—P(HO)(O)—O-5′), triphosphate ((HO)₂(O)P—O—(HO)(O)P—O—P(HO)(O)—O-5′); monothiophosphate (phosphorothioate, (HO)₂(S)P—O-5′), monodithiophosphate (phosphorodithioate; (HO)(HS)(S)P—O-5′), phosphorothiolate ((HO)₂(O)P—S-5′); alpha-thiotriphosphate; beta-thiotriphosphate; gamma-thiotriphosphate; phosphoramidates ((HO)₂(O)P—NH-5′, (HO)(NH₂)(O)P—O-5′), alkylphosphonates [(R^{P})(OH)(O)P—O-5′, R^{P} is optionally substituted C₁₋₃₀ alkyl, e.g., methyl, ethyl, isopropyl, or propyl)], alkyletherphosphonates [(R^{P1})(OH)(O)P—O-5′, R^{P1} is alkoxyalkyl, e.g., methoxymethyl (CH₂OMe) or ethoxymethyl], (HO)₂(X)P—O[—(CH₂)ₐ—O—P(X)(OH)—O]_{b}-5′ or (HO)₂(X)P—O[—(CH₂)ₐ—P(X)(OH)—O]_{b}-5′ or (HO)₂(X)P—[—(CH₂)ₐ—O—P(X)(OH)—O]_{b}—5′, or optionally substituted alkyl, and dialkyl terminal phosphates and phosphate mimics (e.g., HO[—(CH₂)ₐ—O—P(X)(OH)—O]_{b}—5′, H₂N[—(CH₂)ₐ—O—P(X)(OH)—O]_{b}-5′, H[—(CH₂)ₐ—O—P(X)(OH)—O]_{b}-5′, Me₂N[—(CH₂)ₐ—O—P(X)(OH)—O]_{b}-5′, HO[—(CH₂)ₐ—P(X)(OH)—O]_{b}-5′, H₂N[—(CH₂)ₐ—P(X)(OH)—O]_{b}-5′, H[—(CH₂)ₐ—P(X)(OH)—O]_{b}-5′, Me₂N[—(CH₂)ₐ—P(X)(OH)—O]_{b}-5′, wherein
X is O or S;
a and b are each independently 1-10;
each R⁸ and R⁹ is independently H, a targeting ligand (e.g., GalNac), a pharmacokinetics modifier, optionally substituted C₁₋₃₀ alkyl, optionally substituted C₁₋₃₀ alkenyl, or optionally substituted C₁₋₃₀alkynyl;
R^{22′} is —O(CH₂)ₙ₁—C(Y^{M})OR^{LV}, a bond to an internucleotide linkage to a subsequent nucleotide, hydrogen, hydroxyl, protected hydroxyl, halogen, optionally substituted C₁₋₃₀ alkyl, optionally substituted C₂₋₃₀alkenyl, optionally substituted C₂₋₃₀alkynyl, optionally substituted C₁₋₃₀ alkoxy (e.g., methoxy), alkoxyalkyl (e.g., 2-methoxyethyl), alkoxyalkylamine, alkoxyoxycarboxylate, amino, alkylamino, dialkylamino, 5-8 membered heterocyclyl, —O—C₄₋₃₀alkyl-ON(CH₂R⁸)(CH₂R⁹), —O—C₄₋₃₀alkyl-ON(CH₂R⁸)(CH₂R⁹), a ligand, a linker covalently bonded to one or more ligands, a solid support, a linker or a linker covalently bonded to a solid support
R^{LV} is a C₁-C₆alkyl (e.g., ethyl);
R^{23′} is —O(CH₂)ₙ₁—C(Y^{M})OR^{LV}, a bond to an internucleotide linkage to a subsequent nucleotide, hydrogen, hydroxyl, protected hydroxyl, halogen, optionally substituted C₁₋₃₀ alkyl, optionally substituted C₂₋₃₀alkenyl, optionally substituted C₂₋₃₀alkynyl, optionally substituted C₁₋₃₀ alkoxy (e.g., methoxy), alkoxyalkyl (e.g., 2-methoxyethyl), alkoxyalkylamine, alkoxyoxycarboxylate, amino, alkylamino, dialkylamino, 5-8 membered heterocyclyl, —O—C₄₋₃₀alkyl-ON(CH₂R⁸)(CH₂R⁹), —O—C₄₋₃₀alkyl-ON(CH₂R⁸)(CH₂R⁹), a ligand, a linker covalently bonded to one or more ligands, a solid support, a linker or a linker covalently bonded to a solid support;
R^{25′} is —O(CH₂)ₙ₁—C(Y^{M})OR^{LV}, a bond to an internucleotide linkage to a preceding nucleotide, hydrogen, hydroxyl, protected hydroxyl, optionally substituted C₁₋₃₀ alkyl, optionally substituted C₂₋₃₀alkenyl, optionally substituted C₂₋₃₀alkynyl, optionally substituted C₁₋₃₀ alkoxy, optionally substituted 3-8 membered heterocyclyl (e.g., morpholin-1-yl, piperidin-1-yl, or pyrrolidin-1-yl), halogen, alkoxyalkyl (e.g., 2-methoxyethyl), alkoxyalkylamine, alkoxyoxycarboxylate, amino, alkylamino, dialkylamino, —O—C₄₋₃₀alkyl-ON(CH₂R⁸)(CH₂R⁹), —O—C₄₋₃₀alkyl-ON(CH₂R⁸)(CH₂R⁹), vinylphosphonate (VP) group (e.g., ═CH—X^{P}, X^{P} is a phosphate group), C₃₋₆ cycloalkylphosphonate (e.g., cyclopropylphosphonate), monophosphate ((HO)₂(O)P—O-5′), diphosphate ((HO)₂(O)P—O—P(HO)(O)—O-5′), triphosphate ((HO)₂(O)P—O—(HO)(O)P—O—P(HO)(O)—O-5′); monothiophosphate (phosphorothioate, (HO)₂(S)P—O-5′), monodithiophosphate (phosphorodithioate; (HO)(HS)(S)P—O-5′), phosphorothiolate ((HO)₂(O)P—S-5′); alpha-thiotriphosphate; beta-thiotriphosphate; gamma-thiotriphosphate; phosphoramidates ((HO)₂(O)P—NH-5′, (HO)(NH₂)(O)P—O-5′), alkylphosphonates [(R^{P})(OH)(O)P—O-5′, R^{P} is optionally substituted C₁₋₃₀ alkyl, e.g., methyl, ethyl, isopropyl, or propyl)], alkyletherphosphonates [(R^{P1})(OH)(O)P—O-5′, R^{P1} is alkoxyalkyl, e.g., methoxymethyl (CH₂OMe) or ethoxymethyl], (HO)₂(X)P—O[—(CH₂)ₐ—O—P(X)(OH)—O]_{b}-5′ or (HO)₂(X)P—O[—(CH₂)ₐ—P(X)(OH)—O]_{b}-5′ or (HO)₂(X)P—[—(CH₂)ₐ—O—P(X)(OH)—O]_{b}—5′, or optionally substituted alkyl, and dialkyl terminal phosphates and phosphate mimics (e.g., HO[—(CH₂)ₐ—O—P(X)(OH)—O]_{b}—5′, H₂N[—(CH₂)ₐ—O—P(X)(OH)—O]_{b}-5′, H[—(CH₂)ₐ—O—P(X)(OH)—O]_{b}-5′, Me₂N[—(CH₂)ₐ—O—P(X)(OH)—O]_{b}-5′, HO[—(CH₂)ₐ—P(X)(OH)—O]_{b}-5′, H₂N[—(CH₂)ₐ—P(X)(OH)—O]_{b}-5′, H[—(CH₂)ₐ—P(X)(OH)—O]_{b}-5′, Me₂N[—(CH₂)ₐ—P(X)(OH)—O]_{b}-5′, wherein
X is O or S;
a and b are each independently 1-10; and
provided:
(i) at least one of R²², R²³, R²⁴ and R²⁵ is —O(CH₂)ₙ₁—C(Y^{M})N(R^{N′})(R^{N″});
(ii) only one of R²², R²³, R²⁴ and R²⁵ is-O(CH₂)n1-C(O)N(R^{N′})(R^{N″});
(iii) only one of R²² and R²³ is a bond to an internucleotide linkage to a subsequent nucleotide;
(iv) when both of R²² and R²³ are not a bond to an internucleotide linkage to a subsequent nucleotide, then R²⁵ is a bond to an internucleotide linkage to a preceding nucleotide; and
(v) at least one of R^{22′}, R^{23′}, R^{24′} and R^{25′} is —O(CH₂)ₙ₁—C(Y^{M})N(R^{N′})(R^{N″});
(vi) only one of R^{22′}, R^{23′}, R^{24′} and R^{25′} is —O(CH₂)ₙ₁—C(Y^{M})N(R^{N′})(R^{N″});
(vii) only one of R^{22′} and R^{23′} is a bond to an internucleotide linkage to a subsequent nucleotide;
(viii) when both of R^{22′} and R^{23′} are not a bond to an internucleotide linkage to a subsequent nucleotide, then R^{25′} is a bond to an internucleotide linkage to a preceding nucleotide.

262. The method of claim 261, wherein the oligonucleotide comprising the nucleoside of Formula (II′) is linked to a solid support.

263. The method of claim 261, wherein the oligonucleotide comprising the nucleoside of Formula (II′) is not linked to a solid support.

264. The method of claim 261, wherein the oligonucleotide comprising the nucleoside of Formula (II′) is linked to a solid support and the method comprises a step of cleaving the oligonucleotide from the solid support prior to reacting with the amine of formula HN(R^{N′})(R^{N″}).

265. The method of any one of claims 261-264, wherein the oligonucleotide comprises from 3 to 50 nucleotides.

266. The method of any one of claims 261-264, wherein the oligonucleotide comprises at least one ribonucleotide (e.g., 2′-OH).

267. The method of any one of claims 261-266, wherein the oligonucleotide comprises at least one 2′-deoxyribonucleotide.

268. The method of any one of claims 261-267, wherein the oligonucleotide comprises at least one nucleotide with a modified or non-natural nucleobase.

269. The method of any one of claims 261-268, wherein the oligonucleotide comprises at least one nucleotide with a modified ribose sugar.

270. The method of any one of claims 261-269, wherein the oligonucleotide comprises at least one nucleotide comprising a group other than H or OH at the 2′-position of the ribose sugar.

271. The method of any one of claims 261-270, wherein the oligonucleotide comprises at least one nucleotide with a 2′-F ribose.

272. The method of any one of claims 261-271, wherein the oligonucleotide comprises at least one nucleotide with a 2′-OMe ribose.

273. The method of any one of claims 260-272, wherein the oligonucleotide comprises at least one nucleotide comprising a moiety other than a ribose sugar.

274. The method of any one of claims 261-273, wherein the oligonucleotide comprising a nucleoside comprises at least one hydroxyl, phosphate or amino protecting group prior to said reacting step.

275. The method of any one of claims 261-274, wherein the does not comprise a hydroxyl, phosphate or amino protecting group prior to said reacting group.

276. The oligonucleotide of any one of claims 261-275, wherein the oligonucleotide comprises at least one ligand.

277. An oligonucleotide prepared by a method of any one of claims 260-276.

278. The compound of claim 1, wherein R⁴ is —O(CH₂)ₘ₁—X^{M′}—R^{M′} (e.g., —OCH₂CH₂—X^{M′}—R^{M′}).

279. The compound of claim 278, wherein X^{M′} is O.

280. The compound of claim 278, wherein X^{M′} is S.

281. The compound of claim 1, wherein R⁴ is —O(CH₂)ₙ₁—C(Y^{M})N(R^{N′})(R^{N″}).

282. The compound of claim 281, wherein n1 is 1.

283. The compound of claim 282, wherein n1 is 2.

284. The compound of any one of claims 278-283, wherein R² is hydrogen, hydroxyl, protected hydroxyl, halogen, optionally substituted C₁₋₃₀ alkoxy, a reactive phosphorous group, a solid support, a linker, or a linker covalently attached to a solid support.

285. The compound of any one of claims 278-284, wherein R² is hydrogen, hydroxyl, protected hydroxyl, halogen, a reactive phosphorous group, a solid support, a linker, or a linker covalently attached to a solid support.

286. The compound of any one of claims 278-285, wherein R² is hydrogen, hydroxyl, protected hydroxyl, a reactive phosphorous group, a solid support, a linker, or a linker covalently attached to a solid support.

287. The compound of any one of claims 278-286, wherein R² is a reactive phosphorous group, a solid support, a linker, or a linker covalently attached to a solid support.

288. The compound of any one of 278-287, wherein R² is a reactive phosphorous or a linker covalently attached to a solid support.

289. The compound of any one of claims 278-288, wherein R² is a reactive phosphorous group (e.g., a phosphoramidite, such as [(2-cyanoethyl)-(N,N-diisopropyl)]-phosphoramidite or [(ß-thiobenzoylethyl)-(1-pyrrolidinyl)]-thiophosphoramidite.

290. The compound of any one of claims 278-289, wherein R² is a linker covalently attached to a solid support.

291. The compound of any one of claims 278-290, wherein R³ is hydrogen, hydroxyl, protected hydroxyl, halogen, or optionally substituted C₁₋₃₀ alkoxy.

292. The compound of any one of claims 278-291, wherein R³ is hydroxyl or protected hydroxyl.

293. The compound of any one of claims 278-292, wherein R³ is hydrogen, hydroxyl, protected hydroxyl, halogen, optionally substituted C₁₋₃₀ alkoxy, a reactive phosphorous group, a solid support, a linker, or a linker covalently attached to a solid support.

294. The compound of any one of claim 293, wherein R³ is hydrogen, hydroxyl, protected hydroxyl, halogen, a reactive phosphorous group, a solid support, a linker, or a linker covalently attached to a solid support.

295. The compound of any one of claims 293-294, wherein R³ is hydrogen, hydroxyl, protected hydroxyl, a reactive phosphorous group, a solid support, a linker, or a linker covalently attached to a solid support.

296. The compound of any one of claims 293-295, wherein R³ is a reactive phosphorous group, a solid support, a linker, or a linker covalently attached to a solid support.

297. The compound of any one of 293-296, wherein R³ is a reactive phosphorous or a linker covalently attached to a solid support.

298. The compound of any one of claims 292-297, wherein R³ is a reactive phosphorous group (e.g., a phosphoramidite, such as [(2-cyanoethyl)-(N,N-diisopropyl)]-phosphoramidite or [(ß-thiobenzoylethyl)-(1-pyrrolidinyl)]-thiophosphoramidite.

299. The compound of any one of claims 293-298, wherein R³ is a linker covalently attached to a solid support.

300. The compound of any one of claims 293-299, wherein R² is hydrogen, hydroxyl, protected hydroxyl, halogen, or optionally substituted C₁₋₃₀ alkoxy.

301. The compound of any one of claims 293-300, wherein R² is hydroxyl or protected hydroxyl.

302. The compound of any one of claims 293-301, wherein R⁵ is hydroxyl, protected hydroxyl, optionally substituted C₁₋₃₀ alkoxy, vinylphosphonate (VP) group, monophosphate, diphosphate, triphosphate, monothiophosphate (phosphorothioate), monodithiophosphate, phosphorothiolate, alpha-thiotriphosphate, beta-thiotriphosphate, gamma-thiotriphosphate, phosphoramidate, alkylphosphonate, alkyletherphosphonate, dialkyl terminal phosphate or phosphate mimic.

303. The compound of any one of claims 278-302, wherein R⁵ is hydroxyl, protected hydroxyl, vinylphosphonate (VP) group, cyclopropylphosphonate, monophosphate, diphosphate, triphosphate, monothiophosphate (phosphorothioate), monodithiophosphate, phosphorothiolate, alpha-thiotriphosphate, beta-thiotriphosphate, gamma-thiotriphosphate, phosphoramidates, alkylphosphonate, alkyletherphosphonate, dialkyl terminal phosphate, or a phosphate mimic.

304. The compound of any one of claims 278-303, wherein R⁵ is hydroxyl or protected hydroxyl.

305. The compound of claim 1, wherein R⁴ is —O(CH₂)ₙ₁—C(Y^{M})N(R^{N′})(R^{N″}).

306. The compound of claim 305, wherein R³ is a hydroxyl group.

307. The compound of claim 306, wherein R³ is a protected hydroxyl.

308. The compound of claim 307, wherein the protected hydroxyl is —OR^{Pro}, wherein R^{Pro} is selected from the group consisting of acetyl, benzyl, benzoyl, 2,6-dichlorobenzyl, t-butyldimethylsilyl, t-butyldiphenylsilyl, trimethylsilyl, triisopropylsilyl, mesylate, tosylate, 4,4′-dimethoxytrityl (DMT), 9-phenylxanthine-9-yl (Pixyl) and 9-(p-methoxyphenyl)xanthine-9-yl (MOX).

309. The compound of claim 308, wherein the protected hydroxyl is —OR^{Pro}, wherein R^{Pro} is selected from the group consisting of acetyl, benzyl, t-butyldimethylsilyl, t-butyldiphenylsilyl, trimethylsilyl, triisopropylsilyl, and dimethoxytrityl.

310. The compound of claim 309, wherein the protected hydroxyl is —OR^{Pro}, wherein R^{Pro} is selected from the group consisting of t-butyldimethylsilyl, t-butyldiphenylsilyl, trimethylsilyl, and triisopropylsilyl.

311. The compound of claim 310, wherein R³ is a reactive phosphorous group.

312. The compound of claim 311, wherein the reactive phosphorous group is —OP(OR^{P})(N(R^{P2})₂), —OP(SR^{P})(N(R^{P2})₂), —OP(O)(OR^{P})(N(R^{P2})₂), —OP(S)(OR^{P})(N(R^{P2})₂), —OP(O)(SR^{P})(N(R^{P2})₂), —OP(O)(OR^{P})H, —OP(S)(OR^{P})H, —OP(O)(SR^{P})H, —OP(O)(OR^{P})R^{P3}, —OP(S)(OR^{P})R^{P3}, or —OP(O)(SR^{P})R^{P3}.

313. The compound of claim 312, wherein the reactive phosphorous group is —OP(OR^{P})(N(R^{P2})₂).

314. The compound of claim 313, wherein the reactive phosphorous group is OP(OR^{P})(N(R^{P2})₂), wherein R^{P} is cyanoethyl (—CH₂CH₂CN) and each R^{P2} is isopropyl or both R^{P2} taken together with the nitrogen atom to which they are attached form an optionally substituted 3-8 membered heterocyclyl.

315. The compound of claim 305, wherein R³ is a linker or a linker attached to a solid support.

316. The compound of any one of claims 305-315, wherein R² is hydroxyl or protected hydroxyl.

317. The compound of claim 316, wherein the protected hydroxyl is —OR^{Pro}, wherein R^{Pro} is selected from the group consisting of acetyl, benzyl, benzoyl, 2,6-dichlorobenzyl, t-butyldimethylsilyl, t-butyldiphenylsilyl, trimethylsilyl, triisopropylsilyl, mesylate, tosylate, 4,4′-dimethoxytrityl (DMT), 9-phenylxanthine-9-yl (Pixyl) and 9-(p-methoxyphenyl)xanthine-9-yl (MOX).

318. The compound of claim 317, wherein the protected hydroxyl is —OR^{Pro}, wherein R^{Pro} is selected from the group consisting of acetyl, benzyl, t-butyldimethylsilyl, t-butyldiphenylsilyl, trimethylsilyl, triisopropylsilyl, and dimethoxytrityl.

319. The compound of claim 318, wherein the protected hydroxyl is —OR^{Pro}, wherein R^{Pro} is selected from the group consisting of t-butyldimethylsilyl, t-butyldiphenylsilyl, trimethylsilyl, and triisopropylsilyl.

320. The compound of claim 305, wherein R² is a hydroxyl group.

321. The compound of claim 305, wherein R² is a protected hydroxyl.

322. The compound of claim 320, wherein the protected hydroxyl is —OR^{Pro}, wherein R^{Pro} is selected from the group consisting of acetyl, benzyl, benzoyl, 2,6-dichlorobenzyl, t-butyldimethylsilyl, t-butyldiphenylsilyl, trimethylsilyl, triisopropylsilyl, mesylate, tosylate, 4,4′-dimethoxytrityl (DMT), 9-phenylxanthine-9-yl (Pixyl) and 9-(p-methoxyphenyl)xanthine-9-yl (MOX).

323. The compound of claim 321 wherein the protected hydroxyl is —OR^{Pro}, wherein R^{Pro} is selected from the group consisting of acetyl, benzyl, t-butyldimethylsilyl, t-butyldiphenylsilyl, trimethylsilyl, triisopropylsilyl, and dimethoxytrityl.

324. The compound of claim 322, wherein the protected hydroxyl is —OR^{Pro}, wherein R^{Pro} is selected from the group consisting of t-butyldimethylsilyl, t-butyldiphenylsilyl, trimethylsilyl, and triisopropylsilyl.

325. The compound of claim 323, wherein R² is a reactive phosphorous group.

326. The compound of claim 324, wherein the reactive phosphorous group is —OP(OR^{P})(N(R^{P2})₂), —OP(SR^{P})(N(R^{P2})₂), —OP(O)(OR^{P})(N(R^{P2})₂), —OP(S)(OR^{P})(N(R^{P2})₂), —OP(O)(SR^{P})(N(R^{P2})₂), —OP(O)(OR^{P})H, —OP(S)(OR^{P})H, —OP(O)(SR^{P})H, —OP(O)(OR^{P})R^{P3}, —OP(S)(OR^{P})R^{P3}, or —OP(O)(SR^{P})R^{P3}.

327. The compound of claim 325, wherein the reactive phosphorous group is —OP(OR^{P})(N(R^{P2})₂).

328. The compound of claim 326, wherein the reactive phosphorous group is —OP(OR^{P})(N(R^{P2})₂), wherein R^{P} is cyanoethyl (—CH₂CH₂CN) and each R^{P2} is isopropyl or both R^{P2} taken together with the nitrogen atom to which they are attached form an optionally substituted 3-8 membered heterocyclyl.

329. The compound of claim 304, wherein R² is a linker or a linker attached to a solid support.

330. The compound of any one of claims 320-329, wherein R³ is hydroxyl or protected hydroxyl.

331. The compound of claim 330, wherein the protected hydroxyl is —OR^{Pro}, wherein R^{Pro} is selected from the group consisting of acetyl, benzyl, benzoyl, 2,6-dichlorobenzyl, t-butyldimethylsilyl, t-butyldiphenylsilyl, trimethylsilyl, triisopropylsilyl, mesylate, tosylate, 4,4′-dimethoxytrityl (DMT), 9-phenylxanthine-9-yl (Pixyl) and 9-(p-methoxyphenyl)xanthine-9-yl (MOX).

332. The compound of claim 331, wherein the protected hydroxyl is —OR^{Pro}, wherein R^{Pro} is selected from the group consisting of acetyl, benzyl, t-butyldimethylsilyl, t-butyldiphenylsilyl, trimethylsilyl, triisopropylsilyl, and dimethoxytrityl.

333. The compound of claim 332, wherein the protected hydroxyl is —OR^{Pro}, wherein R^{Pro} is selected from the group consisting of t-butyldimethylsilyl, t-butyldiphenylsilyl, trimethylsilyl, and triisopropylsilyl.

334. The compound of any one of claims 305-333, wherein R⁵ is hydroxyl.

335. The compound of any one of claims 305-333, wherein R⁵ is a protected hydroxyl.

336. The compound of any one of claim 335, wherein the protected hydroxyl is —OR^{Pro}, wherein R^{Pro} is an oxygen protecting group.

337. The compound of claim 336, wherein R^{Pro} is selected from the group consisting of acetyl, benzyl, benzoyl, 2,6-dichlorobenzyl, t-butyldimethylsilyl, t-butyldiphenylsilyl, trimethylsilyl, triisopropylsilyl, mesylate, tosylate, 4,4′-dimethoxytrityl (DMT), 9-phenylxanthine-9-yl (Pixyl) and 9-(p-methoxyphenyl)xanthine-9-yl (MOX).

338. The compound of claim 337, wherein R^{Pro} is 4,4′-dimethoxytrityl.

339. The compound of any one of claims 122-183 or 305-338, wherein n1 is 1.

340. The compound of any one of claims 122-183 or 305-338, wherein n1 is 2.

341. The compound of any one of claims 122-183 or 305-340, wherein m1 is 2.
